(19) United States
(12) Patent Application Publication
(10) Pub. No.: US 2024/0246946 A1
(43) Pub. Date: Jul. 25, 2024

(54) MODULATORS OF TREX1

(71) Applicant: Constellation Pharmaceuticals, Inc., Boston, MA (US)

(72) Inventors: Julian R. Levell, Arlington, MA (US); Aaron Coffin, Everett, MA (US); Mary-Margaret Zablocki, Revere, MA (US); Jonathan E. Wilson, Arlington, MA (US); Avinash Khanna, Cambridge, MA (US); David J. Guerin, Cambridge, MA (US); William T. McElroy, Framingham, MA (US); Jennifer L. Rocnik, Cambridge, MA (US)

(21) Appl. No.: 18/288,455

(22) PCT Filed: Apr. 25, 2022
(86) PCT No.: PCT/US2022/026103

§ 371 Date: Oct. 26, 2023

Related U.S. Application Data
(60) Provisional application No. 63/179,723, filed on Apr. 26, 2021.

(51) Int. Cl. C07D 413/14; A61K 31/513

(52) U.S. Cl. CPC C07D 413/14; A61K 31/513

(57) ABSTRACT

Provided are compounds of Formula (I): and pharmaceutically acceptable salts and compositions thereof, which are useful for treating a variety of conditions associated with TREX1.

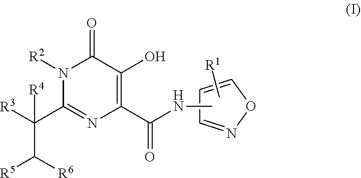

### RELATED APPLICATIONS

This application claims priority to U.S. Provisional Application No. 63/179,723, filed Apr. 26, 2021, the entire contents of which are incorporated herein by reference.

### BACKGROUND

A potential immune therapy is needed for cancers related to the innate immune system recognition of non-self, and to detect and protect against potential danger. Cancer cells differ antigenically from their normal counterparts and emit danger signals to alert the immune system similar to viral infection. These signals, which include damage-associated molecular patterns (DAMPs) and pathogen-associated molecular patterns (PAMPs), further activate the innate immune system resulting in the protection of the host from a variety of threats (Front. Cell Infect. Microbiol. 2012, 2, 168).

Ectopically expressed single stranded DNA (ssDNA) and double stranded DNA (dsDNA) are known PAMPs and/or DAMPs, which are being recognized by the cyclic GMP-AMP synthase (cGAS), a nucleic acid sensor (Nature 2011, 478, 515-518). Upon sensing of cytosolic DNA, cGAS catalyzes the generation of the cyclic dinucleotide 2′,3′-cGAMP, a potent second messenger and activator of the ER transmembrane adapter protein stimulator of interferon genes (STING) (Cell Rep. 2013, 3, 1355-1361). STING activation triggers phosphorylation of IRF3 via TBK1 which in turn leads to type I interferon production and activation of interferon stimulated genes (ISGs); a pre-requisite to the activation of innate immunity and initiation of adaptive immunity. Production of type I interferons thus constitutes a key bridge between the innate and adaptive immunity (Science 2013, 341, 903-906).

Excess type I IFN can be harmful to the host and induce autoimmunity, therefore, negative feedback mechanisms exist that keep type I IFN-mediated immune activation in check. Three prime repair exonuclease I (TREX1) is a 3′-5′ DNA exonuclease responsible for the removal of ectopically expressed ssDNA and dsDNA and is therefore a key repressor of the cGAS/STING pathway (PNAS 2015, 112, 5117-5122).

Type I interferons and downstream pro-inflammatory cytokine responses are critical to the development of immune responses and their effectiveness. Type I interferons enhance both the ability of dendritic cells and macrophages to take up, process, present, and cross-present antigens to T cells, and their potency to stimulate T cells by eliciting the up-regulation of the co-stimulatory molecules such as CD40, CD80 and CD86 (J. Exp. Med. 2011, 208, 2005-2016). Type I interferons also bind their own receptors and activate interferon responsive genes that contribute to activation of cells involved in adaptive immunity (EMBO Rep. 2015, 16, 202-212).

From a therapeutic perspective, type I interferons and compounds that can induce type I interferon production have potential for use in the treatment of human cancers (Nat. Rev Immunol. 2015, 15, 405-414). Interferons can inhibit human tumor cell proliferation directly. In addition, type I interferons can enhance anti-tumor immunity by triggering the activation of cells from both the innate and adaptive immune system. Importantly, the anti-tumor activity of PD-1 blockade requires pre-existing intratumoral T cells. By turning cold tumors into hot and thereby eliciting a spontaneous anti-tumor immunity, type I IFN-inducing therapies have the potential to expand the pool of patients responding to anti-PD-1 therapy as well as enhance the effectiveness of anti-PD1 therapy.

Human and mouse genetic studies suggest that TREX1 inhibition might be amenable to a systemic delivery route and therefore TREX1 inhibitory compounds could play an important role in the anti-tumor therapy landscape. TREX1 is a key determinant for the limited immunogenicity of cancer cells responding to radiation treatment [Trends in Cell Biol., 2017, 27 (8), 543-4; Nature Commun., 2017, 8, 15618]. TREX1 is induced by genotoxic stress and involved in protection of glioma and melanoma cells to anticancer drugs [Biochim. Biophys. Acta, 2013, 1833, 1832-43]. STACT-TREX1 therapy shows robust anti-tumor efficacy in multiple murine cancer models [Glickman et al, Poster P235, 33^{rd} Annual Meeting of Society for Immunotherapy of Cancer, Washington DC, Nov. 7-11, 2018]. (TREX1) expression correlates with cervical cancer cells growth in vitro and disease progression in vivo [Scientific Reports 1019, 9, 351]. Beyond oncology there is also support for agonists of the IFN pathway to be useful in antiviral therapy, for example STING agonists induce an innate antiviral immune response against Hepatitis B Virus via stimulation of the IFN pathway and upregulation of ISG's [Antimicrob. Agents Chemother. 2015, 59:1273-1281] and TREX1 inhibits the innate immune response to HIV type 1 [Nature Immunology, 2010, 11(11), 1005].

### SUMMARY

Provided herein are compounds having the Formula I:

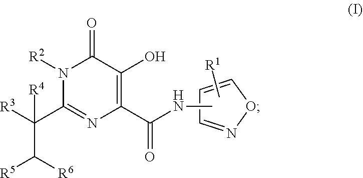

and pharmaceutically acceptable salts and compositions thereof, wherein R¹, R², R³, R⁴, R⁵, and R⁶ are as described herein. The disclosed compounds and compositions modulate TREX1, and are useful in a variety of therapeutic applications such as, for example, in treating cancer.

In one aspect, the disclosed compounds have been found to exhibit profound kinetic properties. See e.g., Table 9.

### DETAILED DESCRIPTION

### 1. General Description of Compounds

In a first embodiment, provided herein is a compound of Formula I:

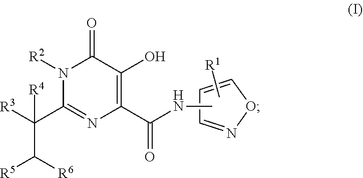

or a pharmaceutically acceptable salt thereof, wherein:
R¹ is halo, hydrogen, (C₁-C₄)alkyl, or halo(C₁-C₄)alkyl;
R² is hydrogen, (C₁-C₄)alkyl, halo(C₁-C₄)alkyl, —(C₁-C₄)alkylOR^{a}, —(C₁-C₄)alkylSR^{a}, or —(C₁-C₄)alkylNR^{b}R^{c};
R^{a} is selected from hydrogen, (C₁-C₄)alkyl, halo(C₁-C₄)alkyl, —COOR^{b}, and —C(O)NR^{b}R^{c};
R^{b} and R^{c} are each independently hydrogen or (C₁-C₄)alkyl;
R³ and R⁴ are each independently hydrogen, halo, (C₁-C₄)alkyl, or halo(C₁-C₄)alkyl;
R⁵ is phenyl, 5 to 7-membered heteroaryl, or 5 to 7-membered heterocyclyl, each of which being optionally substituted with 1 to 3 groups selected from R⁷;
R⁶ is 5 to 7-membered heteroaryl or 5 to 7-membered heterocyclyl, each of which being optionally substituted with 1 to 3 groups selected from R⁸; and
R⁷ and R⁸ are each independently selected from halo, hydroxyl, (C₁-C₄)alkyl, (C₁-C₄)deuteroalkyl, halo(C₁-C₄)alkyl, (C₁-C₄)alkoxy, halo(C₁-C₄)alkoxy, —(C₁-C₄)alkylOR^{a}, —(C₁-C₄)alkylSR^{a}, —(C₁-C₄)alkylNR^{b}R^{c}, —(C₁-C₄)alkyl-cyano, —(C₁-C₄)alkylC(O)NR^{b}R^{c}, cyano, —[(C₁-C₄)alkyl(4- to 7-membered heterocyclyl)], -(4- to 7-membered heterocyclyl), —[(C₁-C₄)alkyl(C₃-C₅)cycloalkyl], —C(O)NR^{b}R^{c}, —COR^{b}, and —COOR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}

### 2. Definitions

When used in connection to describe a chemical group that may have multiple points of attachment, a hyphen (-) designates the point of attachment of that group to the variable to which it is defined. For example, —NHC(O)OR^{a} and —NHC(S)OR^{a} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical. Unless otherwise specified, an alkyl group typically has 1-4 carbon atoms, i.e., (C₁-C₄)alkyl.

The term “deuteroalkyl” when used alone or as part of a larger moiety, such as “halodeuteroalkyl”, and the like, means saturated straight-chain or branched monovalent hydrocarbon radical, wherein one or more of the hydrogen atoms have been replaced by deuterium. Unless otherwise specified, a deuteroalkyl group typically has 1-4 carbon atoms, i.e., (C₁-C₄)deuteroalkyl such as —CD₄ or —CHD₃.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term “heteroaryl” used alone or as part of a larger moiety refers to a 5- to 12-membered (e.g., a 5- to 7-membered) aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered (e.g., a 5- to 7-membered) saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic. Examples of monocyclic saturated or partially unsaturated heterocyclic radicals include, without limitation, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. It will be understood that when specified, optional substituents on a heterocyclyl group may be present on any substitutable position and, include, e.g., the position at which the heterocyclyl is attached.

The term “cycloalkyl” refers to a cyclic hydrocarbon having from, unless otherwise specified, 3 to 10 carbon ring atoms (e.g., a 3 to 5 carbon ring atoms). Cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl may be present on any substitutable position and, include, e.g., the position at which the cycloalkyl or cycloaliphatic group is attached.

The disclosed compounds exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. “R” and “S” represent the configuration of substituents around one or more chiral carbon atoms.

“Racemate” or “racemic mixture” means a compound of equimolar quantities of two enantiomers, wherein such mixtures exhibit no optical activity, i.e., they do not rotate the plane of polarized light.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomer is at least 60%, 70%, 80%, 90%, 99% or 99.9% by weight pure relative to all of the other stereoisomers. The stereoisomeric purity in this case is determined by dividing the total weight in the mixture of the stereoisomers encompassed by the name or structure by the total weight in the mixture of all of the stereoisomers.

When a disclosed compound is named or depicted by structure without indicating the stereochemistry, and the compound has one chiral center, it is to be understood that the name or structure encompasses one enantiomer of compound free from the corresponding optical isomer, a racemic mixture of the compound, or mixtures enriched in one enantiomer relative to its corresponding optical isomer.

When a disclosed compound is named or depicted by structure without indicating the stereochemistry and e.g., the compound has more than one chiral center (e.g., at least two chiral centers), it is to be understood that the name or structure encompasses one stereoisomer free of other stereoisomers, mixtures of stereoisomers, or mixtures of stereoisomers in which one or more stereoisomers is enriched relative to the other stereoisomer(s). For example, the name or structure may encompass one stereoisomer free of other diastereomers, mixtures of stereoisomers, or mixtures of stereoisomers in which one or more diastereomers is enriched relative to the other diastereomer(s).

The term “TREX1” refers to three prime repair exonuclease 1 or DNA repair exonuclease 1, which is an enzyme that in humans is encoded by the TREX1 gene. Mazur D J, Perrino F W (August 1999). “Identification and expression of the TREX1 and TREX2 cDNA sequences encoding mammalian 3′-->5′ exonucleases”. J Biol Chem. 274 (28): 19655-60. doi:10.1074/jbc.274.28.19655. PMID 10391904; Hoss M, Robins P, Naven T J, Pappin D J, Sgouros J, Lindahl T (August 1999). “A human DNA editing enzyme homologous to the Escherichia coli DnaQ/MutD protein”. EMBO J. 18 (13): 3868-75. doi:10.1093/emboj/18.13.3868. PMC 1171463. PMID 10393201. This gene encodes the major 3′->5′ DNA exonuclease in human cells. The protein is a non-processive exonuclease that may serve a proofreading function for a human DNA polymerase. It is also a component of the SET complex, and acts to rapidly degrade 3′ ends of nicked DNA during granzyme A-mediated cell death. Cells lacking functional TREX1 show chronic DNA damage checkpoint activation and extra-nuclear accumulation of an endogenous single-strand DNA substrate. It appears that TREX1 protein normally acts on a single-stranded DNA polynucleotide species generated from processing aberrant replication intermediates. This action of TREX1 attenuates DNA damage checkpoint signaling and prevents pathological immune activation. TREX1 metabolizes reverse-transcribed single-stranded DNA of endogenous retroelements as a function of cell-intrinsic antiviral surveillance, resulting in a potent type I IFN response. TREX1 helps HIV-1 to evade cytosolic sensing by degrading viral cDNA in the cytoplasm.

The term “TREX2” refers to Three prime repair exonuclease 2 is an enzyme that in humans is encoded by the TREX2 gene. This gene encodes a nuclear protein with 3′ to 5′ exonuclease activity. The encoded protein participates in double-stranded DNA break repair, and may interact with DNA polymerase delta. Enzymes with this activity are involved in DNA replication, repair, and recombination. TREX2 is a 3′-exonuclease which is predominantly expressed in keratinocytes and contributes to the epidermal response to UVB-induced DNA damage. TREX2 biochemical and structural properties are similar to TREX1, although they are not identical. The two proteins share a dimeric structure and can process ssDNA and dsDNA substrates in vitro with almost identical k_{cat} values. However, several features related to enzyme kinetics, structural domains, and subcellular distribution distinguish TREX2 from TREX1. TREX2 present a 10-fold lower affinity for DNA substrates in vitro compared with TREX1. In contrast with TREX1, TREX2 lacks a COOH-terminal domain that can mediate protein-protein interactions. TREX2 is localized in both the cytoplasm and nucleus, whereas TREX1 is found in the endoplasmic reticulum, and is mobilized to the nucleus during granzyme A-mediated cell death or after DNA damage.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g. salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, provided herein is a compound of Formula II:

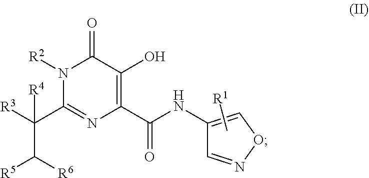

or a pharmaceutically acceptable salt thereof, wherein the variables are as described above for Formula I.

In a third embodiment, R¹ in the compounds of Formula I or II, or a pharmaceutically acceptable salt thereof, is hydrogen, wherein the remaining variables are as described above for Formula I or Formula II.

In a fourth embodiment, R² in the compounds of Formula I or II, or a pharmaceutically acceptable salt thereof, is (C₁-C₄)alkyl, wherein the remaining variables are as described above for Formula I or Formula II or the third embodiment.

In a fifth embodiment, R³ in the compounds of Formula I or II, or a pharmaceutically acceptable salt thereof, is halo, hydrogen or (C₁-C₄)alkyl, wherein the remaining variables are as described above for Formula I or Formula II or the third or fourth embodiment. Alternatively, as part of a fifth embodiment, R³ in the compounds of Formula I or II, or a pharmaceutically acceptable salt thereof, is hydrogen, wherein the remaining variables are as described above for Formula I or Formula II or the third or fourth embodiment.

In a sixth embodiment, R⁴ in the compounds of Formula I or II, or a pharmaceutically acceptable salt thereof, is hydrogen, (C₁-C₄)alkyl, or halo(C₁-C₄)alkyl, wherein the remaining variables are as described above for Formula I or Formula II or the third, fourth, or fifth embodiment. Alternatively, as part of sixth embodiment, R⁴ in the compounds of Formula I or II, or a pharmaceutically acceptable salt thereof, is (C₁-C₄)alkyl or halo(C₁-C₄)alkyl, wherein the remaining variables are as described above for Formula I or Formula II or the third, fourth, or fifth embodiment. Alternatively, as part of sixth embodiment, R⁴ in the compounds of Formula I or II, or a pharmaceutically acceptable salt thereof, is (C₁-C₄)alkyl, wherein the remaining variables are as described above for Formula I or Formula II or the third, fourth, or fifth embodiment.

In a seventh embodiment, R⁵ in the compounds of Formula I or II, or a pharmaceutically acceptable salt thereof, is phenyl or 5 to 7-membered heteroaryl, each of which being optionally substituted with 1 to 3 groups selected from R⁷, wherein the remaining variables are as described above for Formula I or Formula II or the third, fourth, fifth, or sixth embodiment. Alternatively, as part of a seventh embodiment, R⁵ in the compounds of Formula I or II, or a pharmaceutically acceptable salt thereof, is phenyl or pyridyl, each of which being optionally substituted with 1 to 3 groups selected from R⁷, wherein the remaining variables are as described above for Formula I or Formula II or the third, fourth, fifth, or sixth embodiment. Alternatively, as part of a seventh embodiment, R⁵ in the compounds of Formula I or II, or a pharmaceutically acceptable salt thereof, is phenyl optionally substituted with 1 to 3 groups selected from R⁷, wherein the remaining variables are as described above for Formula I or Formula II or the third, fourth, fifth, or sixth embodiment.

In an eighth embodiment, R⁶ in the compounds of Formula I or II, or a pharmaceutically acceptable salt thereof, is 5 to 7-membered heteroaryl optionally substituted with 1 to 3 groups selected from R⁸, wherein the remaining variables are as described above for Formula I or Formula II or the third, fourth, fifth, sixth, or seventh embodiment. Alternatively, as part of an eighth embodiment, R⁶ in the compounds of Formula I or II, or a pharmaceutically acceptable salt thereof, is pyridinyl, oxadiazolyl, triazolyl, tetrazolyl, isoxazolyl, imidazolyl, pyrazolyl, pyrimidinyl, or pyrazinyl, each of which being optionally substituted with 1 to 3 groups selected from R⁸, wherein the remaining variables are as described above for Formula I or Formula II or the third, fourth, fifth, sixth, or seventh embodiment. Alternatively, as part of an eighth embodiment, R⁶ in the compounds of Formula I or II, or a pharmaceutically acceptable salt thereof, is pyrazolyl, pyrimidinyl, or pyrazinyl, optionally substituted with 1 to 3 groups selected from R⁸, each of which being optionally substituted with 1 to 3 groups selected from R⁸, wherein the remaining variables are as described above for Formula I or Formula II or the third, fourth, fifth, sixth, or seventh embodiment. Alternatively, as part of an eighth embodiment, R⁶ in the compounds of Formula I or II, or a pharmaceutically acceptable salt thereof, is pyrazolyl optionally substituted with 1 to 3 groups selected from R⁸, each of which being optionally substituted with 1 to 3 groups selected from R⁸, wherein the remaining variables are as described above for Formula I or Formula II or the third, fourth, fifth, sixth, or seventh embodiment. Alternatively, as part of an eighth embodiment, R⁶ in the compounds of Formula I or II, or a pharmaceutically acceptable salt thereof, is pyrimidinyl optionally substituted with 1 to 3 groups selected from R⁸, each of which being optionally substituted with 1 to 3 groups selected from R⁸, wherein the remaining variables are as described above for Formula I or Formula II or the third, fourth, fifth, sixth, or seventh embodiment. Alternatively, as part of an eighth embodiment, R⁶ in the compounds of Formula I or II, or a pharmaceutically acceptable salt thereof, is pyrazinyl optionally substituted with 1 to 3 groups selected from R⁸, each of which being optionally substituted with 1 to 3 groups selected from R⁸, wherein the remaining variables are as described above for Formula I or Formula II or the third, fourth, fifth, sixth, or seventh embodiment.

In an ninth embodiment, R⁷ and R⁸ in the compounds of Formula I or II, or a pharmaceutically acceptable salt thereof, are each independently selected from halogen, hydroxyl, (C₁-C₄)alkyl, halo(C₁-C₄)alkyl, —(C₁-C₄)alkylOR^{a}, cyano, —(C₁-C₄)alkylNR^{b}R^{c}, —[(C₁-C₄)alkyl(4- to 7-membered heterocyclyl)], —[(C₁-C₄)alkyl(C₃-C₅)cycloalkyl], —(C₁-C₄)alkylNR^{b}R^{c}, —(C₁-C₄)alkyl-cyano, -(4- to 7-membered heterocyclyl), —C(O)NR^{b}R^{c}, and —COR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}, wherein the remaining variables are as described above for Formula I or Formula II or the third, fourth, fifth, sixth, seventh, or eighth embodiment.

In a tenth embodiment, R⁷ in the compounds of Formula I or II, or a pharmaceutically acceptable salt thereof, is selected from halo, (C₁-C₄)alkyl, hydroxyl, halo(C₁-C₄)alkyl, cyano, and —C(O)NR^{b}R^{c}, wherein the remaining variables are as described above for Formula I or Formula II or the third, fourth, fifth, sixth, seventh, eighth, or ninth embodiment. Alternatively, as part of a tenth embodiment, R⁷ in the compounds of Formula I or II, or a pharmaceutically acceptable salt thereof, is selected from halo and cyano, wherein the remaining variables are as described above for Formula I or Formula II or the third, fourth, fifth, sixth, seventh, eighth, or ninth embodiment.

In an eleventh embodiment, R⁸ in the compounds of Formula I or II, or a pharmaceutically acceptable salt thereof, is selected from halo, (C₁-C₄)alkyl, halo(C₁-C₄)alkyl, —(C₁-C₄)alkylOR^{a}, —(C₁-C₄)alkylNR^{b}R^{c}, —[(C₁-C₄)alkyl(4- to 7-membered heterocyclyl)], —[(C₁-C₄)alkyl(C₃-C₅)cycloalkyl], —(C₁-C₄)alkyl-cyano, -(4- to 7-membered heterocyclyl), —(C₁-C₄)alkylNR^{b}R^{c}, and —COR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}, wherein the remaining variables are as described above for Formula I or Formula II or the third, fourth, fifth, sixth, seventh, eighth, ninth, or tenth embodiment. Alternatively, as part of an eleventh embodiment, R⁸ in the compounds of Formula I or II, or a pharmaceutically acceptable salt thereof, is selected from halo, (C₁-C₄)alkyl, halo(C₁-C₄)alkyl, —(C₁-C₄)alkylOR^{a}, —(C₁-C₄)alkylNR^{b}R^{c}, —[(C₁-C₄)alkyl(oxetanyl)], —[(C₁-C₄)alkyl(morpholinyl)], —[(C₁-C₄)alkyl(piperizinyl)], —[(C₁-C₄)alkylcyclopropyl], —(C₁-C₄)alkyl-cyano, -(4- to 7-membered heterocyclyl such as oxetanyl), —(C₁-C₄)alkylNR^{b}R^{c}, and —COR^{b}, wherein said morpholinyl, piperizinyl, and cyclopropyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}, wherein the remaining variables are as described above for Formula I or Formula II or the third, fourth, fifth, sixth, seventh, eighth, ninth, or tenth embodiment. In another alternative, as part of an eleventh embodiment, R⁸ in the compounds of Formula I or II, or a pharmaceutically acceptable salt thereof, is selected from halo, (C₁-C₄)alkyl, halo(C₁-C₄)alkyl, —(C₁-C₄)alkylOR^{a}, —(C₁-C₄)alkylNR^{b}R^{c}, —[(C₁-C₄)alkyl(morpholinyl)], —[(C₁-C₄)alkyl(piperizinyl)], —[(C₁-C₄)alkylcyclopropyl], —(C₁-C₄)alkyl-cyano, -(4- to 7-membered heterocyclyl such as oxetanyl), —(C₁-C₄)alkylNR^{b}R^{c}, and —COR^{b}, wherein said morpholinyl, piperizinyl, and cyclopropyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}, wherein the remaining variables are as described above for Formula I or Formula II or the third, fourth, fifth, sixth, seventh, eighth, ninth, or tenth embodiment.

In a twelfth embodiment, provided is a compound is of the Formula III:

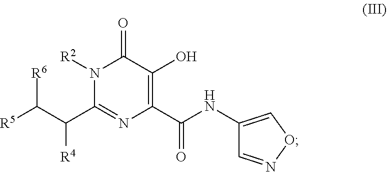

or a pharmaceutically acceptable salt thereof, wherein
R² is (C₁-C₄)alkyl;
R⁴ is (C₁-C₄)alkyl;
R⁵ is phenyl substituted with 1 or 2 groups selected from R⁷,
R⁶ is pyrazolyl, pyrimidinyl or pyrazinyl, optionally substituted with 1 to 3 groups selected from R⁸;
R⁷ is halo, halo(C₁-C₄)alkyl, or cyano;
R⁸ is halo, (C₁-C₄)alkyl, halo(C₁-C₄)alkyl, —(C₁-C₄)alkylOR^{a}, —(C₁-C₄)alkyl-cyano, cyano, —[(C₁-C₄)alkyl(4- to 7-membered heterocyclyl)], -(4- to 7-membered heterocyclyl), —[(C₁-C₄))alkyl(C₃-C₅)cycloalkyl], wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, and halo (C₁-C₄)alkoxy; and
R^{a} is (C₁-C₄)alkyl or halo (C₁-C₄)alkyl.

In a thirteenth embodiment, at least one R⁷, if present, in the compounds of Formula I or II, and at least one R⁷ in the compound of Formula III, or a pharmaceutically acceptable salt thereof, is present at the ortho position, wherein the remaining variables are as described above for Formula I, Formula II, Formula III or the third, fourth, fifth, sixth, seventh, eighth, ninth, tenth, or eleventh embodiment.

In a fifteenth embodiment, R⁷, if present, in the compounds of Formula I or II, and R⁷ in the compound of Formula III, or a pharmaceutically acceptable salt thereof, is chloro or cyano, wherein the remaining variables are as described above for Formula I, Formula II, Formula III or the third, fourth, fifth, sixth, seventh, eighth, ninth, tenth, or eleventh embodiment.

Also provided herein are pharmaceutical compositions comprising a compound of Formula I, Formula II, and Formula III including any of the embodiments described herein or a pharmaceutically acceptable salt thereof, and 2) a pharmaceutically acceptable carrier.

Compounds having the Formula I are further disclosed in the Exemplification and are included in the present disclosure. Pharmaceutically acceptable salts thereof as well as the neutral forms are included.

### 4. Uses, Formulation and Administration

Compounds and compositions described herein are generally useful for modulating the activity of TREX1. In some aspects, the compounds and pharmaceutical compositions described herein inhibit the activity TREX1.

In some aspects, compounds and pharmaceutical compositions described herein are useful in treating a disorder associated with TREX1 function. Thus, provided herein are methods of treating a disorder associated with TREX1 function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with TREX1 function. Also provided is a compound described herein, or a pharmaceutically acceptable salt thereof, or a pharmaceutical composition comprising a disclosed compound or pharmaceutically acceptable salt thereof, for use in treating a disorder associated with TREX1.

In some aspects, the compounds and pharmaceutical compositions described herein are useful in treating cancer.

In some aspects, the cancer treated by the compounds and pharmaceutical compositions described herein is selected from colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, and breast cancer.

In some aspects, the cancer treated by the compounds and pharmaceutical compositions described herein is selected from lung cancer, breast cancer, pancreatic cancer, colorectal cancer, and melanoma.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

The representative examples that follow are intended to help illustrate the present disclosure, and are not intended to, nor should they be construed to, limit the scope of the invention.

General starting materials used were obtained from commercial sources or prepared in other examples, unless otherwise noted.

The following abbreviations have the indicated meanings:
ACN acetonitrile
Ag(Phen)₂OTf Silver(1+), bis(1,10-phenanthroline-κN1,κN10)-, (T-4)-, 1,1,1-trifluoromethanesulfonate
AIBN Azobisisobutyronitrile
BOC t-butyloxycarbonyl
CDI Carbonyldiimidazole
Cs₂CO₃ Cesium Carbonate
DBU 1,8-Diazabicycloundec-7-ene
DCC 1,3-dicyclohexylcarbodiimide
DEA Diethylamine
DCE 1,2-dichloroethane
DCM or CH₂Cl₂ Dichloromethane
DIAD Diisopropyl azodicarboxylate
DIBAL Diisobutyl aluminum hydride
DIPEA or DIEA N,N-diisoproylethylamine, also known as Hunig's base.
DMA N,N-dimethylacetamide
DMAD Dimethyl acetylenedicarboxylate
DMAP 4-(dimethylamino)pyridine
DMF N,N-dimethylformamide
DME 1,2-dimethoxyethane
DMP Dess-Martin periodinane
DMSO Dimethyl sulfoxide
DPPA Diphenylphosphoryl azide
DPPP 1,3-bis(diphenylphosphino)propane
Dtbbpy 4,4′-di-/e/7-butyl-2,2′-dipyridyl
EDC or EDCI 1-(3-dimethylaminopropyl)-3-ethylcarbodiimide hydrochloride
EtOAc Ethyl acetate
EtOH Ethanol
FA Formic Acid
HATU(9-(7-Azabenzotriazol-1-yl)-N, N, N, N-tetramethyluroniumhexafluorophosphate
HCl Hydrochloric acid
HOAt 1-Hydroxy-7-azabenzotriazole or 3H-[1,2,3]triazolo[4,5-b]pyridin-3-ol
HOBt 1-hydroxybenzotriazole
IPA Isopropylamine
iPrMgCl isopropylmagnesium chloride
KHMDS Potassium hexamethyldisilazane
K₂CO₃ Potassium Carbonate
LDA Lithium diisopropylamide
LiBr Lithium Bromide
LiCl Lithium Chloride
LiHMDS or LHMDS Lithium hexamethyldisilazane
LiOH Lithium hydroxide
MCPBA meta-chloroperbenzoic acid
Mel or CH₃I Methyl Iodide
MeOH methanol
MnO₂ Manganese(IV) oxide
MsO Methanefulfonate mesylate
MTBE Methyl t-butyl ether
n-BuLi n-butyllithium
Na₂CO₃ Sodium Carbonate
Na₂SO₄ Sodium Sulphate
NaH Sodium hydride
NaHMDS Sodium hexamethyldisilazane
NaOH Sodium Hydroxide
NBS N-bromosuccinimide
NH₄Cl Ammonium Chloride
NMM 4-methylmorpholine
NMP N-methylpyrrolidinone
PCC Pyridinium chlorochromate
PDC Pyridinium dichromate
Pd(dppf)Cl₂ [1,1′-Bis(diphenylphosphino)ferrocene] dichloropalladium (II)
Pd(dtbpf)Cl₂ [1,1′-Bis(di-tert-butylphosphino)ferrocene] dichloropalladium (II)
Pd(PPh₃)₄ Tetrakis(triphenylphosphine) palladium (0)
rt Room Temperature
SPhos Pd 3G (2-Dicyclohexylphosphino-2′,6′-dimethoxybiphenyl) [2-(2′-amino-1,1′-biphenyl)]palladium(II) methanesulfonate
T₃P 2,4,6-Tripropyl-1,3,5,2,4,6-trioxatriphosphinane 2,4,6-trioxide
TEA Triethylamine
TFA Trifluoroacetic acid
TFAA Trifluoroacetic anhydride
TfO Trifluoromethanesulfonate triflate
THF Tetrahydrofuran
TMSCl Trimethylsilyl chloride
Togni Reagent II 1-Trifluoromethyl-1,2-benziodoxol-3-(1H)-one

The progress of reactions was often monitored by TLC or LC-MS. The LC-MS was recorded using one of the following methods.

LCMS METHOD-1:

| Mobile | (A) | 2 mM Ammonium acetate + 0.1% | | |
|---|---|---|---|---|
| Phase | | Formic Acid in water | | |
| | (B) | 0.1% Formic Acid in acetonitrile | | |
| Column | : | BEH C18 (50*2.1 mm) 1.7 um | | |
| Column | : | 0.55 ml/min | | |
| Flow | | | | |
| Gradient | : | Time (min) | % A | % B |
| | | 0.01 | 98 | 2 |
| | | 0.30 | 98 | 2 |
| | | 0.60 | 50 | 50 |
| | | 1.10 | 25 | 75 |
| | | 2.00 | 0 | 100 |
| | | 2.70 | 0 | 100 |
| | | 2.71 | 98 | 2 |
| | | 3.00 | 98 | 2 |

LCMS Method-2:

| Mobile Phase | (A) | 5 mM Ammonium Acetate + 0.1% | | |
|---|---|---|---|---|
| | | Formic Acid in water | | |
| | (B) | 0.1% Formic Acid in acetonitrile | | |
| Column | : | BEH C18 (50*2.1 mm), 1.7 um or | | |
| | | Equivalent | | |
| Column Flow | : | 0.45 ml/min | | |
| Gradient | : | Time (min) | % A | % B |
| | | 0.01 | 98 | 2 |
| | | 0.50 | 98 | 2 |
| | | 5.00 | 10 | 90 |
| | | 6.00 | 5 | 95 |
| | | 7.00 | 5 | 95 |
| | | 7.01 | 98 | 2 |
| | | 8.00 | 98 | 2 |

LCMS Method-3:

| Mobile Phase | (A) | 5 mM Ammonium | | |
|---|---|---|---|---|
| | | bicarbonate in water | | |
| | (B) | acetonitrile | | |
| Column | : | X-Bridge C18 (50*4.6 mm), | | |
| | | 3.5 um | | |
| Column Flow | : | 1.0 ml/min | | |
| Gradient | : | Time (min) | % A | % B |
| | | 0.01 | 95 | 5 |
| | | 5.00 | 10 | 90 |
| | | 5.80 | 5 | 95 |
| | | 7.20 | 5 | 95 |
| | | 7.21 | 95 | 5 |
| | | 10.00 | 95 | 5 |

LCMS Method-4:

| Mobile Phase | (A) | 10 mM Ammonium | | |
|---|---|---|---|---|
| | | Acetate in WATER | | |
| | (B) | 100% acetonitrile | | |
| Column | : | X-Bridge C18 (150*4.6 mm), | | |
| | | 5 um or Equivalent | | |
| Column Flow | : | 1.0 ml/min | | |
| Gradient | : | Time (min) | % A | % B |
| | | 0.01 | 90 | 10 |
| | | 5.00 | 10 | 90 |
| | | 7.00 | 0 | 100 |
| | | 11.00 | 0 | 100 |
| | | 11.01 | 90 | 10 |
| | | 12.00 | 90 | 10 |

LCMS Method-5:

| Mobile Phase | (A) | 10 mM Ammonium | | |
|---|---|---|---|---|
| | | Acetate in water | | |
| | (B) | 100% acetonitrile | | |
| Column | : | X-Bridge C18 (150*4.6 mm), | | |
| | | 5 um or Equivalent | | |
| Column Flow | : | 1.0 ml/min | | |
| Gradient | : | Time (min) | % A | % B |
| | | 0.01 | 100 | 0 |
| | | 7.00 | 50 | 50 |
| | | 9.00 | 0 | 100 |
| | | 11.00 | 0 | 100 |
| | | 11.01 | 100 | 0 |
| | | 12.00 | 100 | 0 |

LCMS Method-6:

| Mobile Phase | (A) | 0.1% Formic Acid in water | | |
|---|---|---|---|---|
| | (B) | 0.1% Formic Acid in acetonitrile | | |
| Column | : | Zorbax SB-C8 | | |
| | | (4.5 × 75 mm), 3.5 μm | | |
| Column Flow | : | 1.5 ml/min | | |
| Gradient | : | Time (min) | % A | % B |
| | | 0.00 | 95 | 5 |
| | | 3.60 | 5 | 95 |
| | | 4.00 | 5 | 95 |
| | | 4.50 | 95 | 5 |

NMR was recorded at room temperature unless noted otherwise on Varian Inova 400 or 500 MHz spectrometers with the solvent peak used as the reference or on Bruker 300 or 400 MHz spectrometers with the TMS peak used as internal reference.

The compounds described herein may be prepared using the following methods and schemes. Unless specified otherwise, all starting materials used are commercially available.

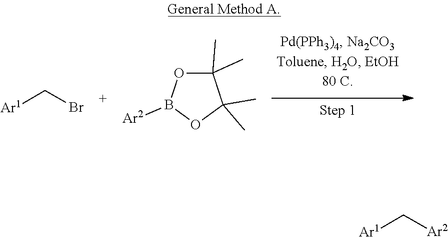

### Synthesis of key intermediate 2-((1-methyl-1H-pyrazol-5-yl)methyl)benzonitrile

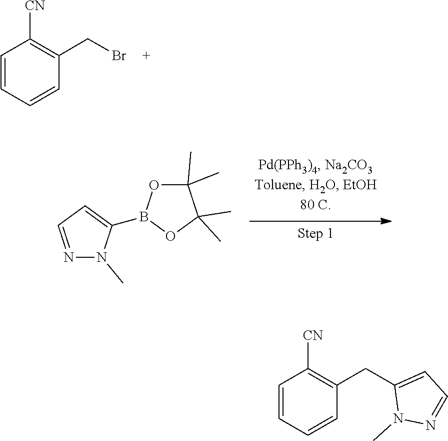

### Step 1: 2-((1-methyl-1H-pyrazol-5-yl)methyl)benzonitrile

A mixture of 2-(bromomethyl)benzonitrile (2.0 g, 10.20 mmol), (1-methyl-1H-pyrazol-5-yl)boronic acid (1.28 g, 10.20 mmol) and sodium carbonate (2.16 g, 20.40 mmol) in a mixture of Toluene:Ethanol:water (7:3:4, 28 ml) was purged with argon gas for 20 minutes. To the reaction mixture, Pd(PPh₃)₄ (0.589 g, 0.51 mmol) was added, and the reaction was purged for 10 minutes. The reaction mixture was heated in a sealed tube at 80° C. for 3 hours. After completion of reaction (monitored by TLC), the reaction mixture was diluted with water (30 ml) and extracted with EtOAc (3×30 ml). The combined organic layer was washed with brine (50 ml), dried over anhydrous sodium sulphate, filtered, and concentrated under reduced pressure. The crude compound was purified using Combi-flash chromatography to give pure title compound (0.450 g, 22%).

LCMS: m/ [symbol] 198.1 [M⁺+1].

¹H NMR (400 MHz, DMSO-d₆): δ 3.77 (s, 3H), 4.23 (s, 2H), 5.85 (d, J=1.2 Hz, 1H), 7.31 (d, J=1.6 Hz, 1H), 7.37 (d, J=8.0 Hz, 1H), 7.48 (t, J=7.6 Hz, 1H), 7.69 (t, J=7.6 Hz, 1H), 7.86 (dd, J=6.8, 0.8 Hz, 1H).

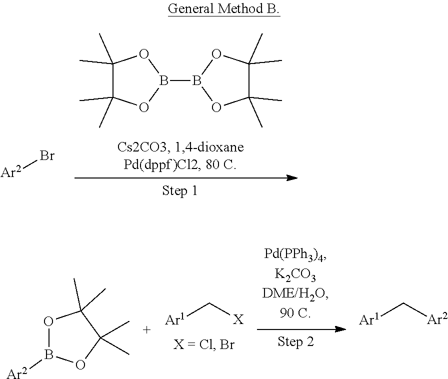

### Synthesis of key intermediate 1,3-dimethyl-4-(4,4,5,5-tetramethyl-1,3,2-dioxaborolan-2-yl)-1H-pyrazole

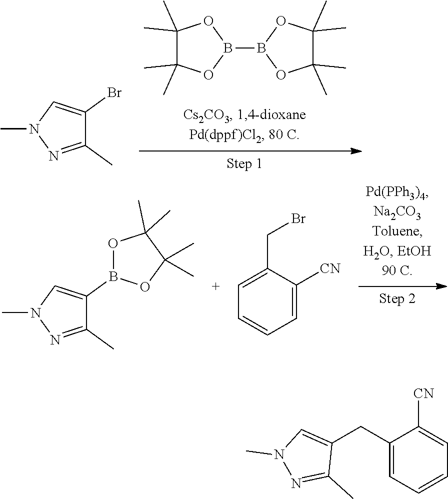

### Step 1: 1,3-dimethyl-4-(4,4,5,5-tetramethyl-1,3,2-dioxaborolan-2-yl)-1H-pyrazole

A mixture of 4-bromo-1,3-dimethyl-1H-pyrazole (3.0 g, 17.14 mmol), 4,4,4′,4′,5,5,5′,5′-octamethyl-2,2′-bi(1,3,2-dioxaborolane) (8.67 g, 34.42 mmol) and cesium carbonate (13.92 g, 42.85 mmol) in dioxane (60 ml) was purged for 20 minutes with argon gas. Pd(dppf)Cl₂ (1.25 g, 1.71 mmol) was added, and the reaction was purged for 10 minutes.

The reaction mixture was heated in a sealed tube at 80° C. for 2 hours. After completion of reaction (monitored by TLC), the reaction mixture was filtered through Celite bed and filtrate was washed with EtOAc (3×50 ml). The combined organic layer was washed with brine (50 ml), dried over anhydrous sodium sulphate, and concentrated under reduced pressure. The crude compound was purified by using Combi-flash chromatography to obtain pure title compound (1.40 g, 36%).

LCMS: m/ [symbol] 223.3 [M⁺+1].

¹H NMR (400 MHz, DMSO-d₆): δ 1.07 (s, 12H), 1.32 (s, 3H), 3.93 (s, 3H), 7.94 (s, 1H).

### Step 2: 2-((1,3-dimethyl-1H-pyrazol-4-yl)methyl)benzonitrile

A mixture of 2-(bromomethyl)benzonitrile (1.23 g, 6.27 mmol), 1,3-dimethyl-4-(4,4,5,5-tetramethyl-1,3,2-dioxaborolan-2-yl)-1H-pyrazole (1.39 g, 6.27 mmol) and sodium carbonate (1.33 g, 12.6 mmol) in a mixture of Toluene:Ethanol:water (7:3:4, 20 ml) was purged for 20 minutes with argon gas. Pd(PPh₃)₄ (0.363 g, 0.315 mmol) was added and purged for 10 minutes. The reaction mixture was heated in a sealed tube at 90° C. for 2 hours. After completion of reaction (monitored by TLC), the reaction mixture was filtered through Celite bed and Celite bed was washed with EtOAc (3×50 ml). The organic layer was separated, and the aqueous layer was extracted with EtOAc (2×100 ml). The combined organic layer was washed with brine (50 ml), dried over anhydrous sodium sulphate, and concentrated under reduced pressure. The crude compound was purified by using Combi-flash chromatography to obtain pure title compound (0.785 g, 59%).

LCMS: m/ [symbol] 212.3 [M⁺+1].

¹H NMR (400 MHz, DMSO-d₆): δ 2.07 (s, 3H), 3.70 (s, 3H), 3.90 (s, 2H), 7.32 (s, 1H), 7.38-7.44 (m, 2H), 7.65 (t, J=7.6 Hz, 1H), 7.80 (d, J=7.6 Hz, 1H).

### Synthesis of Key intermediate 2-((3,5-dimethyl-1-((4-(trifluoromethyl)phenyl)sulfonyl)-1H-pyrazol-4-yl)methyl) benzonitrile

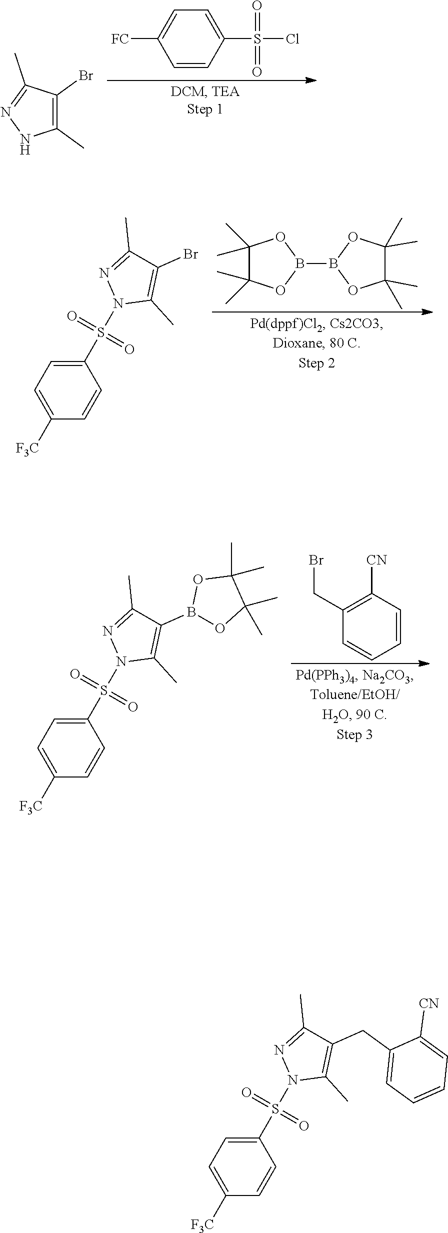

### Step 1: 4-Bromo-3,5-dimethyl-1-((4-(trifluoromethyl)phenyl)sulfonyl)-1H-pyrazole

To an ice-cold solution of 4-Bromo-3,5-dimethyl-1H-pyrazole (7 g, 40.0 mmol) in Dichloromethane (70 ml) was added TEA (7.23 ml, 52.0 mmol) under Nitrogen atmosphere at 0° C. To the above reaction mixture, 4-(Trifluoromethyl)benzenesulfonyl chloride (10.76 g, 44.0 mmol) was added portion wise at 0° C. The reaction mixture was further stirred for overnight at room temperature. After completion of reaction (monitored by TLC), the reaction mixture was quenched with water (100 ml) and extracted with Dichloromethane (2×150 ml). The combined organic layer was washed with brine (70 ml), dried over anhydrous sodium sulphate, and concentrated under reduced pressure. The crude compound was used in the next step without further purification.

LCMS: m/ [symbol] 385.1 [M⁺+2].

¹H NMR (400 MHz, CDCl₃): δ 2.24 (s, 3H), 2.55 (s, 3H), 7.82 (d, J=8.4 Hz, 2H), 8.11 (d, J=8.4H [symbol] , 2H).

### Step 2: 3,5-Dimethyl-4-(4,4,5,5-tetramethyl-1,3,2-dioxaborolan-2-yl)-1-((4-(trifluoromethyl) phenyl)sulfonyl)-1H-pyrazole

A mixture of 4-Bromo-3,5-dimethyl-1-((4-(trifluoromethyl)phenyl)sulfonyl)-1H-pyrazole (5.0 g, 13.0 mmol), 4,4,4′,4′,5,5,5′,5′-Octamethyl-2,2′-bi(1,3,2-dioxaborolane) (6.62 g, 26.0 mmol) and Cesium carbonate (10.62 g, 32.6 mmol) in Dioxane (50 ml) was purged for 20 minutes with Argon gas. Pd(dppf)Cl₂ (0.954 g, 1.30 mmol) was added, and purging was continued for another 10 minutes. The reaction mixture was heated in a sealed tube at 80° C. for 2 hours. After completion of reaction (monitored by TLC), the reaction mixture was filtered through Celite bed and filtrate was washed with EtOAc (3×50 ml). The combined organic layer was washed with brine (50 ml), dried over anhydrous sodium sulphate, and concentrated under reduced pressure. The crude compound was purified by using Combi-flash chromatography to obtain pure title compound (1.80 g, 31%).

LCMS m/ [symbol] : 431.3 [M⁺+1].

¹H NMR (400 MHz, CDCl₃): δ 1.29 (s, 12H), 2.32 (s, 3H), 2.71 (s, 3H) 7.80 (d, J=8.0 H [symbol] 2H), 8.12 (d, J=8.4 Hz, 2H).

### Step 3: 2-((3,5-dimethyl-1-((4-(trifluoromethyl)phenyl)sulfonyl)-1H-pyrazol-4-yl)methyl) benzonitrile

A mixture of 3,5-Dimethyl-4-(4,4,5,5-tetramethyl-1,3,2-dioxaborolan-2-yl)-1-((4-(trifluoromethyl) phenyl)sulfonyl)-1H-pyrazole (15 g, 34.86 mmol), 2-(Bromomethyl)benzonitrile (6.83 g, 34.86 mmol) and sodium carbonate (9.22 g, 87.15 mmol) were combined in Toluene:Ethanol:water (7:3:3, 195 ml), the solution was purged for 20 minutes with Argon gas. Pd(PPh₃)₄ (2.013 g, 1.74 mmol) was added, and purging was continued for another 10 minutes. The reaction mixture was heated in a sealed tube at 90° C. for 3 hours. After completion of reaction (monitored by TLC), the reaction mixture was filtered through Celite bed and filtrate was washed with EtOAc (3×70 ml). The combined organic layer was washed with brine (100 ml), dried over anhydrous sodium sulphate, and concentrated under reduced pressure. The crude compound was purified by using SiO₂ column chromatography to obtain pure title compound (8.5 g, 58%).

LCMS m/ [symbol] : 420.16 [M⁺+1].

¹H NMR (400 MHz, CDCl₃): δ 2.06 (s, 3H), 2.50 (s, 3H), 3.91 (s, 2H), 6.94 (d, J=8.0 Hz, 1H), 7.34 (t, J=7.6 Hz, 1H), 7.48 (t, J=7.6 Hz, 1H), 7.66 (d, J=7.2 Hz, 1H) 7.82 (d, J=8.4 Hz, 2H), 8.12 (d, J=8.4 Hz, 2H).

The following Key Intermediates in Table 1 were prepared according to the General Methods described above.

TABLE 1

| Structure | ¹H NMR | LCMS |
|---|---|---|
| 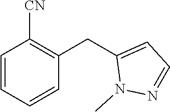 | 400 MHz, DMSO-d₆): δ 3.77 (s, 3H), 4.23 (s, 2H), 5.85 (d, J = 1.2 Hz, 1H), 7.31 (d, J = 1.6 Hz, 1H), 7.37 (d, J = 8.0 Hz, 1H), 7.48 (t, J = 7.6 Hz, 1H), 7.69 (t, J = 7.6 Hz, 1H), 7.86 (dd, J = 6.8, 0.8 Hz, 1H). | m/z 198.1 [M⁺ + 1] |
| 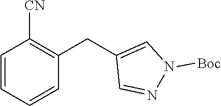 [SMILES: CC(C)(C)OC(=O)n1cc(Cc2ccccc2C#N)cn1] | 400 MHz, DMSO-d₆): δ 8.08 (d, J = 0.9 Hz, 1H), 7.82 (dd, J = 7.8, 1.4 Hz, 1H), 7.72-7.61 (m, 2H), 7.54-7.39 (m, 2H), 1.56 (s, 9H). | m/z 589.2 [2M + Na] |
| 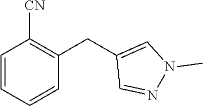 | 400 MHz, DMSO-d₆): δ 3.76 (s, 3H), 3.95 (s, 2H), 7.27 (s, 1H), 7.39-7.48 (m, 3H), 7.64 (t, J = 7.6 Hz, 1H), 7.78 (d, J = 7.6 Hz, 1H). | m/z 197.7 [M⁺ + 1] |
| 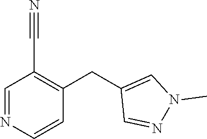 | ¹H NMR (400 MHz, DMSO-d₆): δ 3.77 (s, 3H), 3.98 (s, 2H), 7.31 (s, 1H), 7.48 (d, J = 4.8 Hz, 2H), 8.74 (d, J = 4.8 Hz, 1H), 8.95 (s, 1H). | m/z 199.1 [M⁺ + 1] |
| 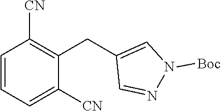 [SMILES: CC(C)(C)OC(=O)n1cc(Cc2c(C#N)cccc2C#N)cn1] | | m/z 209.2 [M − Boc + H]⁺ |
| 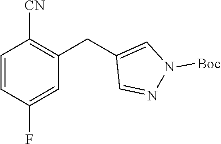 [SMILES: CC(C)(C)OC(=O)n1cc(Cc2cc(F)ccc2C#N)cn1] | | m/z 302.2 [M + H]⁺ |
| 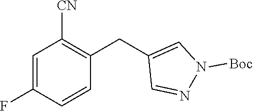 [SMILES: CC(C)(C)OC(=O)n1cc(Cc2ccc(F)cc2C#N)cn1] | | m/z 365.1 [M + Na + MeCN]⁺ |
| 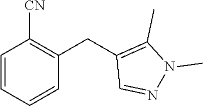 [SMILES: Cc1c(Cc2ccccc2C#N)cnn1C] | 400 MHz, CDCl₃): δ 2.23 (s, 3H), 3.81 (s, 3H), 3.98 (s, 2H), 7.26-7.29 (m, 2H), 7.31 (t, J = 7.6 Hz, 1H), 7.49-7.53 (m, 1H), 7.65 (dd, J = 1.2 Hz, 7.6 Hz, 1H). | m/z 212.2 [M⁺ + 1] |
| 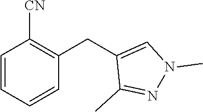 [SMILES: Cc1nn(C)cc1Cc1ccccc1C#N] | 400 MHz, DMSO-d₆): δ 2.07 (s, 3H), 3.70 (s, 3H), 3.90 (s, 2H), 7.32 (s, 1H), 7.38-7.44 (m, 2H), 7.65 (t, J = 7.6 Hz, 1H), 7.80 (d, J = 7.6 Hz, 1H). | m/z 212.3 [M⁺ + 1] |
| 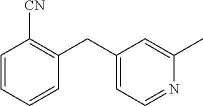 [SMILES: Cc1cc(Cc2ccccc2C#N)ccn1] | 400 MHz, DMSO-d₆) δ 8.35 (d, 1H), 7.85 (d, 1H), 7.71-7.67 (m, 1H), 7.53-7.45 (m, 2H), 7.09 (s, 1H), 7.01 (d, 1H), 4.15 (s, 2H), 2.41 (s, 3H). | |
| 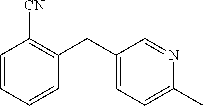 [SMILES: Cc1ccc(Cc2ccccc2C#N)cn1] | | m/z 208.9 [M + H] |
| 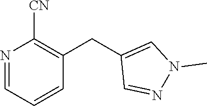 | 400 MHz, DMSO-d₆): δ 3.89 (s, 3H), 4.07 (s, 2H), 7.28 (s, 1H), 7.29 (s, 1H), 7.44-7.47 (m, 1H), 7.68 (d, J = 7.6 Hz, 1H), 8.59 (dd, J = 4.8 Hz and 1.2 Hz, 1H) | m/z 198.9 [M⁺ + 1] |
| 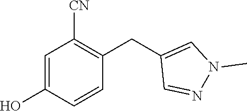 [SMILES: Cn1cc(Cc2ccc(O)cc2C#N)cn1] | | |
| 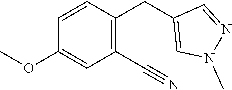 [SMILES: COc1ccc(Cc2cnn(C)c2)c(C#N)c1] | ¹H NMR (400 MHz, DMSO-d₆): δ 3.76 (s, 3H), 3.79 (s, 3H), 3.87 (s, 2H), 7.45 (s, 1H), 7.37-7.36 (m, 2H), 7.24-7.21 (m, 2H). | m/z 228.5 [M⁺ + 1] |
| 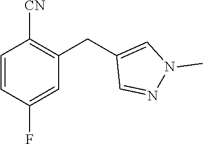 | ¹H NMR (400 MHz, DMSO-d₆): δ 3.77 (s, 3H), 3.84 (s, 2H), 7.12 (td, J = 8.4 Hz, 3.2 Hz, 1H), 7.20 (dd, J = 9.6 Hz, 3.2 Hz, 1H), 7.29 (s, 1H), 7.47 (dd, J = 8.8 Hz, 5.6 Hz, 1H), 7.50 (s, 1H). | m/z 225.1 [M⁺ + 1] |
| 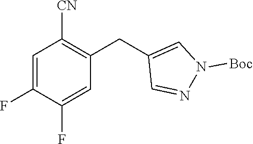 [SMILES: CC(C)(C)OC(=O)n1cc(Cc2cc(F)c(F)cc2C#N)cn1] | 300 MHz, DMSO-d₆): δ 8.21-8.06 (m, 2H), 7.74-7.60 (m, 2H), 4.01 (s, 2H), 1.58 (s, 9H). | m/z 220.1 [M − Boc + H] |
| 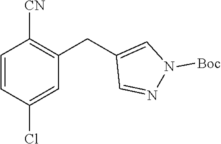 [SMILES: CC(C)(C)OC(=O)n1cc(Cc2cc(Cl)ccc2C#N)cn1] | 400 MHz, DMSO-d₆): δ 8.13 (s, 1H), 7.88 (d, J = 8.3 Hz, 1H), 7.72 (s, 1H), 7.63 (d, J = 2.1 Hz, 1H), 7.54 (dd, J = 8.3, 2.2 Hz, 1H), 4.03 (s, 2H), 1.56 (s, 9H). | m/z 259.1 [M − Boc + MeCN + H] |
| 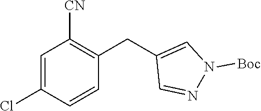 [SMILES: CC(C)(C)OC(=O)n1cc(Cc2ccc(Cl)cc2C#N)cn1] | 300 MHz, DMSO-d₆): δ 8.10 (d, J = 0.9 Hz, 1H), 8.05-7.93 (m, 1H), 7.80-7.66 (m, 2H), 7.51 (d, J = 8.4 Hz, 1H), 4.02 (s, 2H), 1.56 (s, 9H). | m/z 259.0 [M − Boc + MeCN + H] |
| 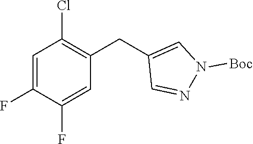 [SMILES: CC(C)(C)OC(=O)n1cc(Cc2cc(F)c(F)cc2Cl)cn1] | | m/z 229.1 [M − Boc + H] |
| 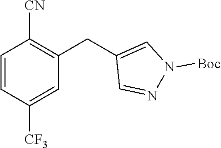 [SMILES: CC(C)(C)OC(=O)n1cc(Cc2cc(C(F)(F)F)ccc2C#N)cn1] | 300 MHz, DMSO-d₆): δ 8.14 (d, J = 0.9 Hz, 1H), 8.09 (d, J = 8.1 Hz, 1H), 7.94 (d, J = 1.9 Hz, 1H), 7.88-7.81 (m, 1H), 7.73 (d, J = 0.8 Hz, 1H), 4.14 (s, 2H), 1.56 (s, 9H). | m/z 293.1 [M − Boc + MeCN + H] |
| 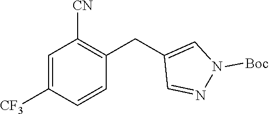 [SMILES: CC(C)(C)OC(=O)n1cc(Cc2ccc(C(F)(F)F)cc2C#N)cn1] | 300 MHz, DMSO-d₆): δ 8.40-8.25 (m, 1H), 8.15 (d, 1H), 8.04 (dd, 1H), 7.79-7.61 (m, 2H), 4.14 (s, 2H), 1.56 (s, 9H). | m/z 252.0 [M − Boc + MeCN + H] |
| 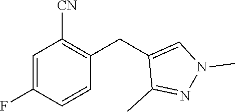 [SMILES: Cc1nn(C)cc1Cc1ccc(F)cc1C#N] | ¹H NMR (400 MHz, DMSO-d₆): δ 2.07 (s, 3H), 3.70 (s, 3H), 3.88 (s, 2H), 7.32 (s, 1H), 7.43 (dd, J = 8.8 Hz, 5.6 Hz, 1H), 7.55 (td, J = 8.8 Hz, 2.8 Hz, 1H), 7.82 (dd, J = 8.8 Hz, 2.8 Hz, 1H). | m/z 230.1 [M ⁺ + 1] |
| 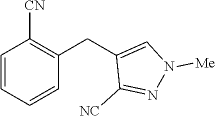 [SMILES: Cn1cc(Cc2ccccc2C#N)c(C#N)n1] | 400 MHz, DMSO-d₆) δ 7.84 (dd, J = 7.8, 1.4 Hz, 1H), 7.79 (s, 1H), 7.69 (m, 1H), 7.50-7.43 (m, 2H), 4.10 (s, 2H), 3.90 (s, 3H). | m/z 223.3 [M + H] |
| 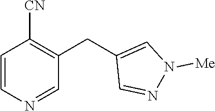 [SMILES: Cn1cc(Cc2cnccc2C#N)cn1] | 400 MHz, DMSO-d₆): δ 3.77 (s, 3H), 3.98 (s, 2H), 7.31 (s, 1H), 7.47-7.54 (m, 2H), 8.74 (d, J = 4.8 Hz, 1H), 8.95 (s, 1H) | m/z 199.0 [M⁺ + 1] |
| 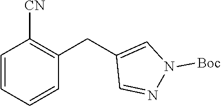 [SMILES: CC(C)(C)OC(=O)n1cc(Cc2ccccc2C#N)cn1] | | m/z 293.2 [M⁺ + 1] |
| 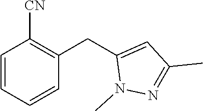 [SMILES: Cc1cc(Cc2ccccc2C#N)n(C)n1] | ¹H NMR (400 MHz, DMSO-d₆): δ 2.06 (s, 3H), 3.70 (s, 3H), 4.18 (s, 2H), 5.62 (s, 1H), 7.40 (d, J = 8.0 Hz, 1H), 7.48 (t, J = 7.6 Hz, 1H), 7.69 (t, J = 7.6 Hz, 1H), 7.86 (d, J = 7.6 Hz, 1H). | m/z 212.1 [M⁺ + 1] |
| 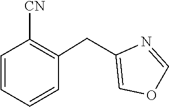 [SMILES: N#Cc1ccccc1Cc1cocn1] | ¹H NMR (400 MHz, DMSO-d₆): δ 4.06 (s, 2H), 7.50-7.41 (m, 2H), 7.67 (t, J = 6.8 Hz, 1H), 7.82 (d, J = 7.6 Hz, 1H), 7.91 (s, 1H), 8.32 (s, 1H). | m/z 185.2 [M⁺ + 1] |
| 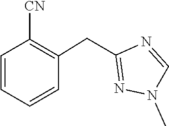 | ¹H NMR (400 MHz, DMSO-d₆): δ 3.79 (s, 3H), 4.16 (s, 2H), 7.48-7.41 (m, 2H), 7.64 (td, J = 8.0 Hz, 1.2 Hz, 1H), 7.80 (d, J = 8.0 Hz, 1H), 8.35 (s, 1H). | m/z 199.1 [M⁺ + 1] |
| 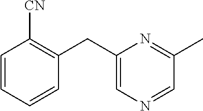 [SMILES: Cc1cncc(Cc2ccccc2C#N)n1] | ¹H NMR (400 MHz, CDCl₃): δ 2.56 (s, 3H), 4.41 (s, 2H), 7.39 (t, J = 7.6 Hz, 1H), 7.44 (d, J = 8.0 Hz, 1H), 7.58 (t, J = 7.6 Hz, 1H), 7.71 (d, J = 7.6 Hz, 1H), 8.37 (d, J = 4.0 Hz, 2H). | m/z 210.1 [M⁺ + 1] |
| 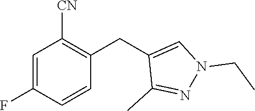 [SMILES: CCn1cc(Cc2ccc(F)cc2C#N)c(C)n1] | ¹H NMR (400 MHz, DMSO-d₆): δ 1.30 (t, J = 7.2 Hz, 3H), 2.07 (s, 3H), 3.88 (s, 2H), 3.98 (q, J = 7.2 Hz, 2H), 7.45-7.38 (m, 2H), 7.55-7.53 (m, 1H), 8.83-7.81 (m, 1H). | m/z 244.2 [M⁺ + 1] |
| 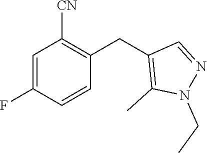 [SMILES: CCn1ncc(Cc2ccc(F)cc2C#N)c1C] | ¹H NMR (400 MHz, DMSO-d₆): δ 1.26 (bs, 3H), 2.20 (s, 3H), 3.88 (s, 2H), 4.01-4.02 (m, 2H), 7.15 (s, 1H), 7.42 (bs, 1H), 7.52 (bs, 1H), 7.79 (d, J = 8.0 Hz, 1H). | m/z 244.2 [M⁺ + 1] |
| 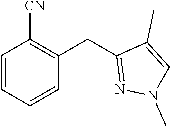 [SMILES: Cc1cn(C)nc1Cc1ccccc1C#N] | ¹H NMR (400 MHz, DMSO-d₆): δ 1.91 (s, 3H), 3.69 (s, 3H), 4.01 (s, 2H), 7.33 (d, J = 7.6, Hz, 1H), 7.42 (d, J = 8.0 Hz, 2H), 7.61 (t, J = 7.6 Hz, 1H), 7.78 (d, J = 7.6 Hz, 1H). | m/z 212.1 [M⁺ + 1] |
| 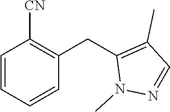 [SMILES: Cc1cnn(C)c1Cc1ccccc1C#N] | 1H NMR (400 MHz, DMSO-d6): δ 1.87 (s, 3H), 3.65 (s, 3H), 4.23 (s, 2H), 7.04 (d, J = 7.2 Hz, 1H), 7.24 (s, 1H), 7.46 (t, J = 7.6 Hz, 1H), 7.64 (t, J = 7.6 Hz, 1H), 7.86 (d, J = 7.2 Hz, 1H). | m/z 212.0 [M⁺ + 1] |
| 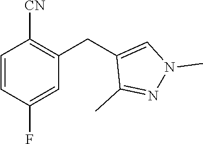 [SMILES: Cc1nn(C)cc1Cc1cc(F)ccc1C#N] | ¹H NMR (400 MHz, DMSO-d₆): δ 2.07 (s, 3H), 3.69 (s, 3H), 3.89 (s, 2H), 7.23-7.32 (m, 3H), 7.90-7.93 (m, 1H). | m/z 230.1 [M⁺ + 1] |
| 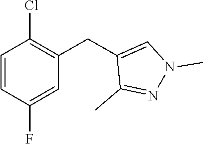 [SMILES: Cc1nn(C)cc1Cc1cc(F)ccc1Cl] | ¹H NMR (400 MHz, DMSO-d₆): δ 2.05 (s, 3H), 3.69 (s, 3H), 3.80 (s, 2H), 7.48 (dd, J = 8.8 Hz, 5.2 Hz, 1H), 7.32 (s, 1H), 7.14-7.04 (m, 2H). | m/z 239.1 [M⁺ + 1] |
| 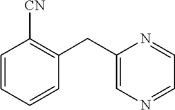 [SMILES: N#Cc1ccccc1Cc1cnccn1] | ¹H NMR (400 MHz, DMSO-d₆): δ 4.39 (s, 2H), 7.46 (t, J = 7.6 Hz, 1H), 7.50 (d, J = 10.8 Hz, 1H), 7.67 (t, J = 7.6 Hz, 1H), 7.84 (d, J = 7.6 Hz, 1H), 8.52 (d, J = 10.8 Hz, 2H), 8.68 (s, 1H). | m/z 195.8 [M⁺ + 1] |
| 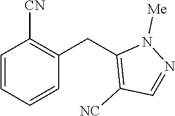 [SMILES: Cn1ncc(C#N)c1Cc1ccccc1C#N] | 400 MHz, DMSO-d₆): δ 7.76 (s, 1H), 7.73 (dd, J = 7.7 Hz, 1.4 Hz, 1H), 7.60 (td, J = 7.8 Hz, 1.5 Hz, 1H), 7.47-7.41 (m, 1H), 7.23-7.19 (m, 1H) 4.39 (s, 2H), 3.82 (s, 3H) | |
| 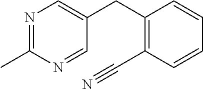 [SMILES: Cc1ncc(Cc2ccccc2C#N)cn1] | ¹H NMR (400 MHz, DMSO-d₆): δ 2.58 (s, 3H), 4.17 (s, 2H), 7.44 (t, J = 7.6 Hz, 1H), 7.51 (d, J = 7.2 Hz, 1H), 7.68 (t, J = 8 Hz, 1H), 7.82 (d, J = 7.6 Hz, 1H), 8.59 (s, 2H). | m/z 209.9 [M⁺ + 1] |
| 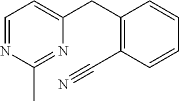 [SMILES: Cc1nccc(Cc2ccccc2C#N)n1] | ¹H NMR (400 MHz, DMSO-d6): δ 2.55 (s, 3H), 4.28 (s, 2H), 7.14 (d, J = 4.8 Hz, 1H), 7.46-7.52 (m, 2H), 7.68 (t, J = 8.0 Hz, 1H), 7.84 (d, J = 7.6 Hz, 1H), 8.61 (d, J = 5.2 Hz, 1H). | m/z 209.9 [M⁺ + 1] |
| 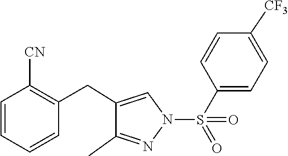 [SMILES: Cc1nn(S(=O)(=O)c2ccc(C(F)(F)F)cc2)cc1Cc1ccccc1C#N] | | m/z 406.1 [M⁺ + 1] |
| 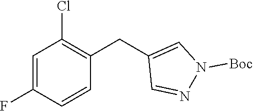 [SMILES: CC(C)(C)OC(=O)n1cc(Cc2ccc(F)cc2Cl)cn1] | | m/z 211.1 [M − Boc + H] |
| 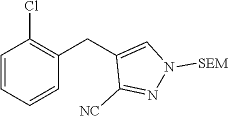 [SMILES: C[Si](C)(C)CCOCn1cc(Cc2ccccc2Cl)c(C#N)n1] | ¹H NMR (400 MHz, DMSO-d₆) δ 8.03 (d, 1H), 7.61-7.48 (m, 1H), 7.47-7.30 (m, 3H), 5.52 (s, 2H), 4.08 (s, 2H), 3.61-3.57 (m, 2H), 0.93-0.78 (m, 2H), 0.14 (s, 9H) | |
| 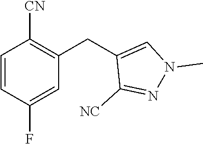 [SMILES: Cn1cc(Cc2cc(F)ccc2C#N)c(C#N)n1] | | m/z 241.1 [M⁺ + 1] |
| 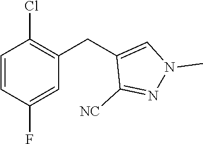 [SMILES: Cn1cc(Cc2cc(F)ccc2Cl)c(C#N)n1] | | m/z 250.1 [M⁺ + 1] |
| 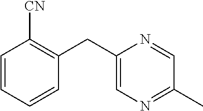 [SMILES: Cc1cnc(Cc2ccccc2C#N)cn1] | | m/z 210.1 [M⁺ + 1] |
| 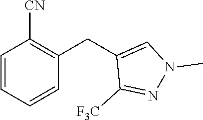 [SMILES: Cn1cc(Cc2ccccc2C#N)c(C(F)(F)F)n1] | | m/z 266.1 [M⁺ + 1] |
| 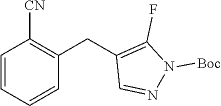 [SMILES: CC(C)(C)OC(=O)n1ncc(Cc2ccccc2C#N)c1F] | | m/z 202.3 [M − Boc + H] |
| 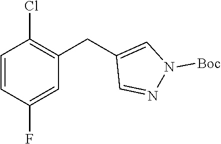 [SMILES: CC(C)(C)OC(=O)n1cc(Cc2cc(F)ccc2Cl)cn1] | | m/z 202.3 [M − Boc + MeCN |
| 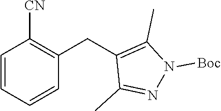 [SMILES: Cc1nn(C(=O)OC(C)(C)C)c(C)c1Cc1ccccc1C#N] | | m/z 312.1 [M⁺ + 1] |
| 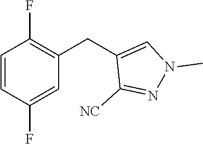 [SMILES: Cn1cc(Cc2cc(F)ccc2F)c(C#N)n1] | | m/z 234.1 [M⁺ + 1] |
| 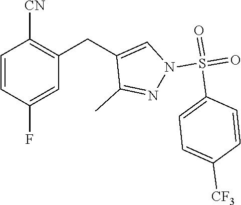 [SMILES: Cc1nn(S(=O)(=O)c2ccc(C(F)(F)F)cc2)cc1Cc1cc(F)ccc1C#N] | | m/z 423.9 [M⁺ + 1] |
| 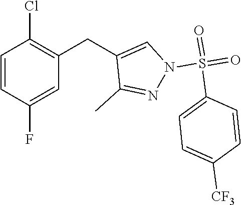 [SMILES: Cc1nn(S(=O)(=O)c2ccc(C(F)(F)F)cc2)cc1Cc1cc(F)ccc1Cl] | | m/z 432.9 [M⁺ + 1] |
| 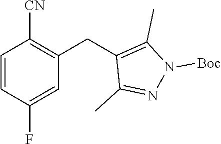 [SMILES: Cc1nn(C(=O)OC(C)(C)C)c(C)c1Cc1cc(F)ccc1C#N] | | m/z 330.1 [M⁺ + 1] |
| 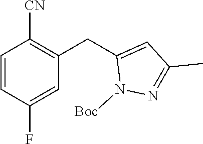 [SMILES: Cc1cc(Cc2cc(F)ccc2C#N)n(C(=O)OC(C)(C)C)n1] | | m/z 216.0 [M − Boc + H] |
| 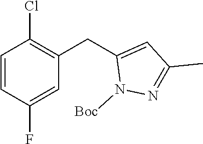 [SMILES: Cc1cc(Cc2cc(F)ccc2Cl)n(C(=O)OC(C)(C)C)n1] | | m/z 325.0 [M⁺ + 1] |
| 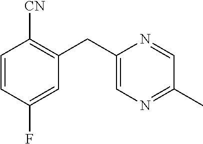 [SMILES: Cc1cnc(Cc2cc(F)ccc2C#N)cn1] | | m/z 228.1 [M⁺ + 1] |
| 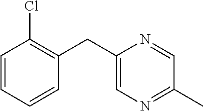 | | m/z 219.0 [M⁺ + 1] |
| 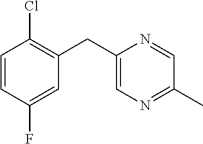 [SMILES: Cc1cnc(Cc2cc(F)ccc2Cl)cn1] | | m/z 237.0 [M⁺ + 1] |
| 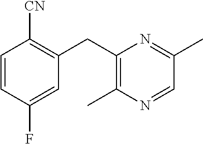 [SMILES: Cc1cnc(C)c(Cc2cc(F)ccc2C#N)n1] | | m/z 242.0 [M⁺ + 1] |
| 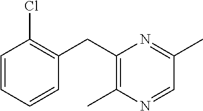 [SMILES: Cc1cnc(C)c(Cc2ccccc2Cl)n1] | | m/z 233.0 [M⁺ + 1] |
| 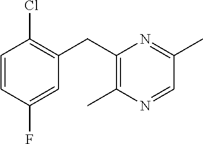 [SMILES: Cc1cnc(C)c(Cc2cc(F)ccc2Cl)n1] | | m/z 251.0 [M⁺ + 1] |
| 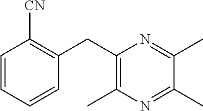 [SMILES: Cc1nc(C)c(Cc2ccccc2C#N)nc1C] | | m/z 238.1 [M⁺ + 1] |
| 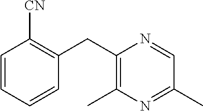 [SMILES: Cc1cnc(Cc2ccccc2C#N)c(C)n1] | | m/z 224.0 [M⁺ + 1] |
| 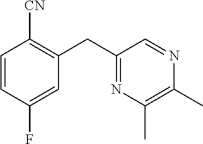 [SMILES: Cc1ncc(Cc2cc(F)ccc2C#N)nc1C] | | m/z 242.2 [M⁺ + 1] |
| 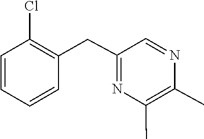 [SMILES: Cc1ncc(Cc2ccccc2Cl)nc1C] | | m/z 233.1 [M⁺ + 1] |
| 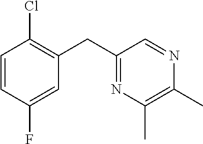 [SMILES: Cc1ncc(Cc2cc(F)ccc2Cl)nc1C] | | m/z 251.1 [M⁺ + 1] |

### Synthesis of 3-cyano-N,N-dimethyl-4-((1-((4-(trifluoromethyl)phenyl)sulfonyl)-1H-pyrazol-4-yl)methyl)benzamide

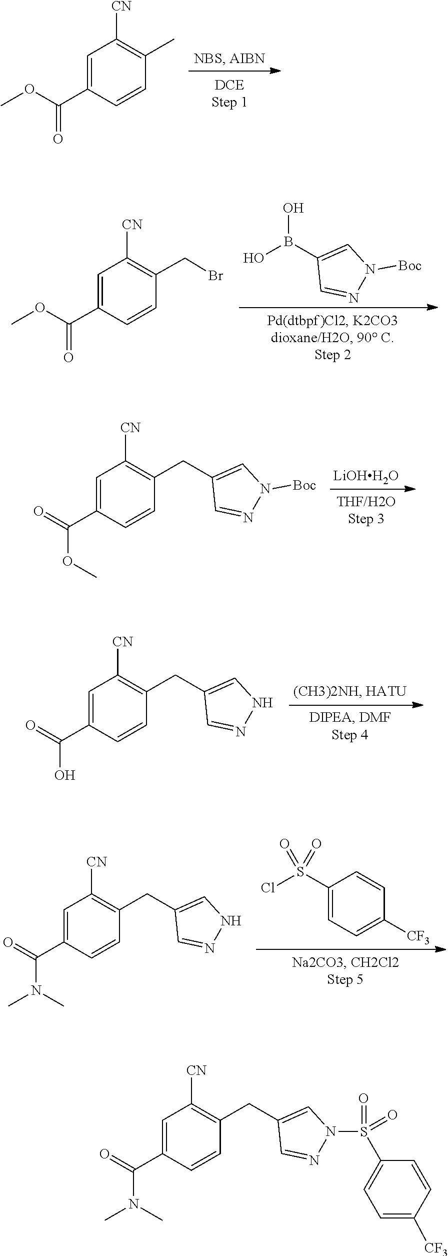

### Step 1: methyl 4-(bromomethyl)-3-cyanobenzoate

To a stirred solution of methyl 3-cyano-4-methylbenzoate (8.00 g, 45.71 mmol) in DCM (80 mL) was added NBS (8.95 g, 50.28 mmol) and 2,2-azobisisobutyronitrile (2.25 g, 13.71 mmol). The reaction mixture was heated to 80° C. and stirred for 4 h and which point it was cooled to rt and diluted with water. The product was extracted with DCM and the combined organic layers were dried over Na₂SO₄ then concentrated in vacuo. The resulting crude material was purified by silica gel column chromatography (50% EtOAc/hexane). Fractions containing product were collected and concentrated in vacuo giving the desired product as a yellow oil (8.1 g, 70% yield)

¹H NMR (400 MHz, DMSO-d6): δ 8.36 (s, 1H), 8.24 (m, 1H), 7.88 (d, 1H), 4.87 (s, 2H), 3.89 (s, 3H).

### Step 2: tert-butyl 4-(2-cyano-4-(methoxycarbonyl)benzyl)-1H-pyrazole-1-carboxylate

To a mixture of methyl 4-(bromomethyl)-3-cyanobenzoate (8.90 g, 35.2 mmol), (1-(tert-butoxycarbonyl)-1H-pyrazol-4-yl)boronic acid (8.95 g, 42.2 mmol) and K₃PO₄ (14.9 g, 70.4 mmol) in dioxane (90 mL) and water was added Pd(dtbpf)Cl₂ (2.29 g, 3.52 mmol). The resulting mixture was heated to 60° C. and stirred for 2 hours at which point it was cooled to rt and concentrated in vacuo. The material was then diluted with water and the product was extracted with DCM, the combined organic layers were washed with brine then dried over Na₂SO₄ and concentrated in vacuo. The resulting crude material was purified by silica gel column chromatography (98% DCM/petroleum ether). Fractions containing product were collected and concentrated in vacuo giving the desired product as a yellow oil (7.5 g, 62% yield)

ESI-MS m/ [symbol] : m/ [symbol] 283.3 [M-Boc+MeCN+H]+

### Step 3: 2-[(2-cyanophenyl)(phenyl)methoxy]-5-methoxy-1-methyl-N-(1,2-oxazol-4-yl)-6-oxopyrimidine-4-carboxamide

To a 0° C. solution of tert-butyl 4-(2-cyano-4-(methoxycarbonyl)benzyl)-1H-pyrazole-1-carboxylate (3.0 g, 8.80 mmol) dissolved in THF (30 mL) and water (6 mL) was added LiOH·H₂O (0.961 g, 22.8 mmol) portion wise. The resulting mixture was then warmed to rt and stirred for 3 h at which point it was concentrated in vacuo. The resulting aq. solution was then acidified to pH 3 with HCl (2M) and the product was isolated by reverse phase chromatography (0% to 100% acetonitrile/water (0.1% FA) fractions containing product were combined and concentrated to afford the product as an off-white solid (1.97 g, 99% yield)

ESI-MS m/ [symbol] : m/ [symbol] 228.1 [M+H]⁺

### Step 4: 4-((1H-pyrazol-4-yl)methyl)-3-cyano-N,N-dimethylbenzamide

To a 0° C. stirred solution of 4-((1H-pyrazol-4-yl)methyl)-3-cyanobenzoic acid (1.97 g, 8.68 mmol), dimethylamine hydrochloride (7.07 g, 86.8 mmol eq) and HATU (5.02 g, 13.2 mmol) in DMF (20 mL) was added Hunig's base (17.05 g, 132.2 mmol) dropwise. The mixture was stirred at room temperature for 1 h at which point the mixture was diluted with water and product was extracted with DCM. The organic layer was washed with brine, dried over Na₂SO₄ then concentrated in vacuo giving the desired product (1.50 g) which was used in subsequent steps with no further purification.

ESI-MS m/ [symbol] : m/ [symbol] 255.2 [M+H]⁺

### Step 5: 3-cyano-N,N-dimethyl-4-((1-((4-(trifluoromethyl)phenyl)sulfonyl)-1H-pyrazol-4-yl)methyl)benzamide

To a mixture of 4-((1H-pyrazol-4-yl)methyl)-3-cyano-N,N-dimethylbenzamide (3.40 g, 13.4 mmol) and Na₂CO₃ (2.84 g, 26.8 mmol) in DCM (35 mL) was added a solution of 4-(trifluoromethyl)benzenesulfonyl chloride (4.25 g, 17.4 mmol) in DCM (10 mL) dropwise at 0° C. The resulting reaction was stirred at rt overnight and then poured into ice water and the product was extracted with DCM. The organic layer was collected, washed with brine then dried over Na₂SO₄ and concentrated in vacuo. The resulting crude material was purified by silica gel column chromatography (2:1 DCM/EtOAc). Fractions containing product were collected and concentrated in vacuo giving the desired product as a white solid (5.6 g, 90% yield)

ESI-MS m/ [symbol] : m/ [symbol] 463.1 [M+H]⁺

### Synthesis of 2-((2-Methyl-2H-tetrazol-5-yl)methyl)benzonitrile and 5-(2-bromobenzyl)-1-methyl-1H-tetrazole

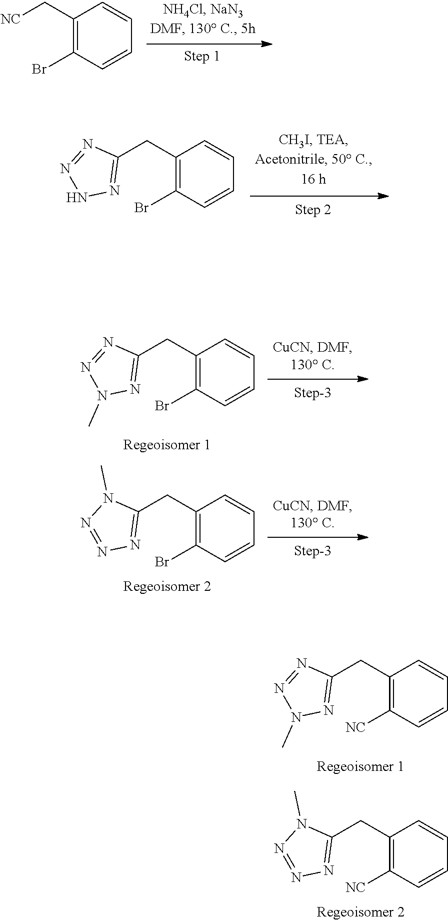

### Step 1: 5-(2-Bromobenzyl)-2H-tetrazole

A mixture of 2-(2-bromophenyl)acetonitrile (15 g, 76.9 mmol), NaN₃ (10.0 g, 153.9 mmol) and NH₄Cl (8.23 g, 153.9 mmol) in DMF (150 ml) was stirred at 130° C. for 5 hours. After completion of reaction (monitored by TLC), the reaction mixture was poured on to ice cold water (30 ml) and extracted with EtOAc (2×30 ml). The combined organic layer was washed with brine (50 ml), dried over anhydrous sodium sulphate, filtered, and evaporated under vacuum to obtain crude title compound. (17.3 g, 94%). The crude compound used in the next step without further purification.

LCMS: m/ [symbol] 239.0 [M+1], 241.0 [M+2].

¹H NMR (400 MHz, DMSO-d6): δ 4.39 (s, 2H), 7.24-7.29 (m, 1H), 7.39-7.42 (m, 2H), 7.64 (d, J=8.0 Hz, 1H), 16.20 (bs, 1H).

### Step 2: 5-(2-Bromobenzyl)-2-methyl-2H-tetrazole (Regioisomer 1) and 5-(2-Bromobenzyl)-1-methyl-1H-tetrazole (Regioisomer 2)

To a stirred solution of 5-(2-bromobenzyl)-2H-tetrazole (17.3 g, 72.36 mmol) in acetonitrile (170 ml) was added triethylamine (11.2 ml, 79.59 mmol). To the resulting reaction mixture, methyl iodide (5.40 ml, 86.8 mmol) was added at room temperature. The reaction mixture was stirred at 50° C. for 16 hours. After completion of reaction (monitored by TLC), the reaction mixture was evaporated under vacuum to obtain the crude compound. The crude compound was purified using column chromatography to obtain pure title compounds (7.5 g (Regioisomer 1), and 8.6 g (Regioisomer 2), 87.91%).

Regioisomer 1: ¹H NMR (400 MHz, DMSO-d6): δ 4.35 (s, 3H), 4.43 (s, 2H), 7.15-7.20 (m, 1H), 7.31-7.35 (m, 2H), 7.61 (d, J=7.6 Hz, 1H).

Regioisomer 2: ¹H NMR (400 MHz, DMSO-d6): δ 3.97 (s, 3H), 4.46 (s, 2H), 7.16 (d, J=6.0 Hz, 1H), 7.22 (t, J=7.6 Hz, 1H), 7.33 (t, J=7.6 Hz, 1H), 7.65 (d, J=6.8 Hz, 1H).

### Step 3a: 2-((2-Methyl-2H-tetrazol-5-yl)methyl)benzonitrile

A mixture of 5-(2-bromobenzyl)-2-methyl-2H-tetrazole (Regioisomer 1) (1 g, 3.9 mmol) and CuCN (1.76 g, 19.8 mmol) in DMF (10 ml) was heated at 130° C. for 16 hours. After completion of reaction (monitored by TLC), the reaction mixture was poured on to ice cold water (30 ml) and reaction mixture was filtered. The filtrate was extracted with EtOAc (2×30 ml). Combined organic layer was washed with brine (50 ml), dried over anhydrous sodium sulphate, filtered, and evaporated under vacuum to obtain the crude compound. The crude compound was purified using column chromatography to obtain pure title compound (0.350 g, 44%).

Regioisomer 1: ¹H NMR (400 MHz, DMSO-d6): δ 4.34 (s, 3H), 4.50 (s, 2H), 7.39 (d, J=7.6 Hz, 1H), 7.43 (d, J=8.8 Hz, 1H), 7.57 (t, J=7.6 Hz, 1H), 7.69 (d, J=7.6 Hz, 1H).

### Step 3b: 2-((1-Methyl-1H-tetrazol-5-yl)methyl)benzonitrile

A mixture of 5-(2-bromobenzyl)-1-methyl-1H-tetrazole (Regioisomer 2) (1 g, 3.98 mmol) and CuCN (1.76 g, 19.8 mmol) in DMF (10 ml) was heated at 130° C. for overnight. After completion of reaction (monitored by TLC), the reaction mixture was poured on to ice cold water (30 ml) and filtered the reaction mixture. Then filtrate was extracted with EtOAc (2×30 ml). Combined organic layer was washed with brine (50 ml), dried over anhydrous sodium sulphate, filtered, and evaporated under vacuum to obtain the crude compound. The crude compound was purified using column chromatography to obtain pure title compound (0.180 g, 23%).

Regioisomer 2: ¹H NMR (400 MHz, DMSO-d6): δ 4.09 (s, 3H), 4.53 (s, 2H), 7.46-7.50 (m, 2H), 7.65 (t, J=8.0 Hz, 1H), 7.74 (d, J=7.6 Hz, 1H).

### Synthesis of 2-((5,6-dimethylpyrazin-2-yl)methyl)benzonitrile

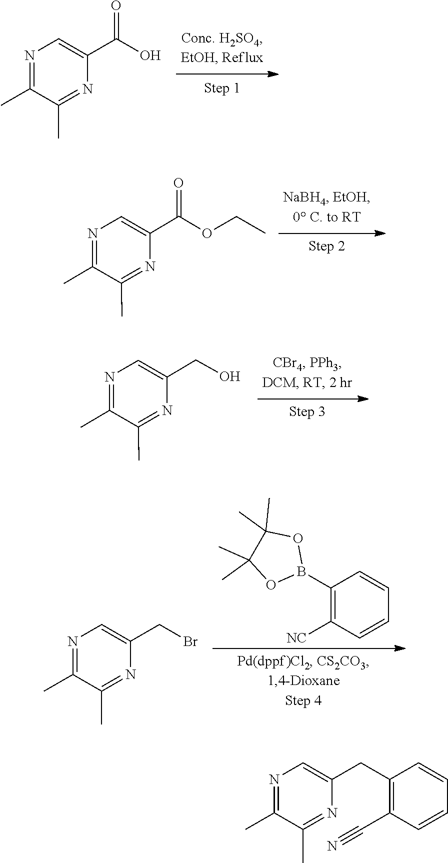

### Step 1: Ethyl 5,6-dimethylpyrazine-2-carboxylate

To a stirred solution of 5,6-dimethylpyrazine-2-carboxylic acid (8.0 g, 52.5 mmol) in Ethanol (80 ml) Conc. H₂SO₄ (3.5 ml) was added dropwise at 0° C. The reaction mixture was heated at 60° C. for overnight. After completion of reaction (monitored by TLC), solvent was evaporated from the reaction mixture. The reaction mixture was quenched with saturated aqueous NaHCO₃ (30 ml) and extracted with EtOAc (2×30 ml). Organic layer was washed with brine (50 ml), dried over anhydrous sodium sulphate, filtered, and evaporated under vacuum to obtain crude title compound. The crude compound was used for next step without further purification.

LCMS: m/ [symbol] 180.8[M+].

### Step 2: (5,6-dimethylpyrazin-2-yl)methanol

To a stirred solution of ethyl 5,6-dimethylpyrazine-2-carboxylate (6.0 g, 33.3 mmol) in Ethanol (60 ml), NaBH₄ (2.5 g, 66.6 mmol) was added portion wise at 0° C. and reaction mixture was stirred at room temperature for overnight. After completion of reaction (monitored by TLC), solvent was evaporated. The crude compound was purified using column chromatography to obtain pure title compound (3.3 g, 72%).

LCMS: m/ [symbol] 130.8 [M+]

¹H NMR (400 MHz, DMSO-d6): δ 2.45 (s, 6H), 4.54 (d, J=4.0 Hz, 2H), 5.47 (t, J=3.6 Hz, 1H), 8.34 (s, 1H).

### Step 3: 5-(bromomethyl)-2,3-dimethylpyrazine

To a stirred solution of 5,6-dimethylpyrazin-2-yl)methanol (3.2 g, 23.0 mmol) and Carbon tetrabromide (8.4 g, 3 mmol) in DCM (32 ml) was added PPh₃ (6.68 g, 25.0 mmol) at 0° C. Reaction mixture was stirred at room temperature for 2 hours. After completion of reaction (monitored by TLC), solvent was evaporated. The crude compound purified using column chromatography to obtain pure title compound (3.0 g, 65%).

LCMS: m/ [symbol] 201.8 [M+1].

### Step 4: 2-((5,6-dimethylpyrazin-2-yl)methyl)benzonitrile

A suspension of 2-(4,4,5,5-tetramethyl-1,3,2-dioxaborolan-2-yl)benzonitrile (0.120 g, 0.6 mmol), 5-(bromomethyl)-2,3-dimethylpyrazine (0.205 g, 0.9 mmol), Dioxane (1.5 ml) and Cesium carbonate (0.205 g, 0.9 mmol) was purged with Argon gas for 20 minutes. PdCl2(dppf) (0.043 g, 0.06 mmol) was added, and purging was continued for another 10 minutes. The reaction mixture was heated in a sealed tube at 90° C. for 2 hours. After completion of reaction (monitored by TLC), the reaction mixture was filtered through Celite bed and was washed with EtOAc (3×60 ml). The combined organic layer was washed with brine (20 ml), dried over anhydrous sodium sulphate, and concentrated under reduced pressure. The crude compound was purified by using Combi-flash chromatography to obtain pure title compound (0.045 g, 33%).

LCMS: m/ [symbol] 224.0 [M+1].

¹H NMR (400 MHz, DMSO-d6): δ 2.41-2.44 (m, 6H), 4.27 (s, 2H), 7.42-7.47 (m, 2H), 7.65 (t, J=7.6 Hz, 1H), 7.82 (d, J=7.6 Hz, 1H), 8.27 (s, 1H).

### Synthesis of 2-((3,6-dimethylpyrazin-2-yl)methyl)benzonitrile

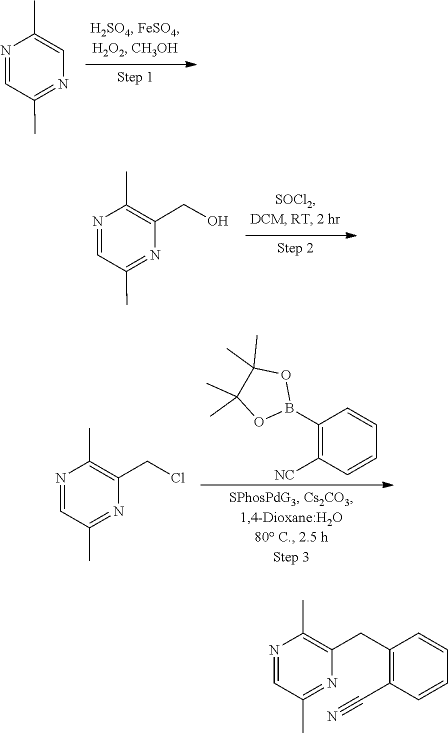

### Step 1: (3,6-dimethylpyrazin-2-yl)methanol

A stirred solution of ferrous sulphate heptahydrate (25.74 g, 92.6 mmol) in methanol (100 ml) was added 2,5-dimethylpyrazine (10 g, 92.6 mmol) and Sulfuric acid (50 ml) dropwise at 0° C. Reaction mixture was stirred at 0° C. for 30 minutes. To it, 30% Hydrogen peroxide in water (70 ml) was carefully added under ice cooling and stirring was continue for 4 hours. After completion of reaction (monitored by TLC), pH of the reaction mixture was adjusted to 12 with aqueous sodium hydroxide solution and extracted with EtOAc (4×300 ml). Organic layer was washed with brine (500 ml), dried over anhydrous sodium sulphate, filtered, and evaporated under vacuum to obtain the crude compound. The crude compound was purified using column chromatography to obtain pure title compound. (3.5 g, 27%).

LCMS: m/ [symbol] 139.12 [M+1].

¹H NMR (400 MHz, DMSO-d6): δ 2.43 (s, 6H), 5.56 (d, J=5.6 Hz, 2H), 5.24 (t, J=5.6 Hz, 1H), 8.03 (s, 1H).

### Step 2: 3-(chloromethyl)-2,5-dimethylpyrazine

To a stirred solution of (3,6-dimethylpyrazin-2-yl) methanol (3.5 g, 25.4 mmol) in DCM (35 ml) was dropwise added SOCl₂ (6.0 ml, 50.7 mmol) at 0° C. The reaction mixture was warmed to room temperature and stirred for 2 hours. After completion of reaction (monitored by TLC), solvent was evaporated under vacuum to obtain crude title compound (3.35 g). The crude compound was used in the next step without further purification.

LCMS: m/ [symbol] 157.05 [M+1].

### Step 3: 2-((3,6-dimethylpyrazin-2-yl)methyl)benzonitrile

A mixture of 3-(chloromethyl)-2,5-dimethylpyrazine (3.5 g, 22.4 mmol), 2-(4,4,5,5-tetramethyl-1,3,2-dioxaborolan-2-yl)benzonitrile (7.70 g, 33.7 mmol) and Cs₂CO₃ (18.22 g, 56.1 mmol) in 1,4-Dioxane (40 ml) was purged with Argon for 10 minutes. SPhosPdG3 (0.919 g, 0.2 mmol) was added and reaction mixture was again purged with Argon for 10 minutes. The reaction mixture was heated at 90° C. for 2 hours. After completion of reaction (monitored by TLC), the reaction mixture was poured into ice-cold water (100 ml) and extracted with EtOAc (3×100 ml). Combined organic layer was washed with brine (100 ml), dried over sodium sulfate, filtered, and evaporated under vacuum to obtain crude compound. The crude compound was purified using column chromatography to obtain pure title compound (2.65 g, 52%).

LCMS: m/ [symbol] 223.8 [M+1].

¹H NMR (400 MHz, DMSO-d6): δ 2.35 (s, 6H), 4.34 (s, 2H), 7.30 (d, J=6.0 Hz, 1H), 7.44 (t, J=7.6 Hz, 1H), 7.63 (t, J=6.8 Hz, 1H), 7.84 (d, J=11.4 Hz, 1H), 8.28 (s, 1H).

### Synthesis of N-{4-[(2-cyanophenyl)methyl]-1-methylpyrazol-3-yl}-N-methylacetamide

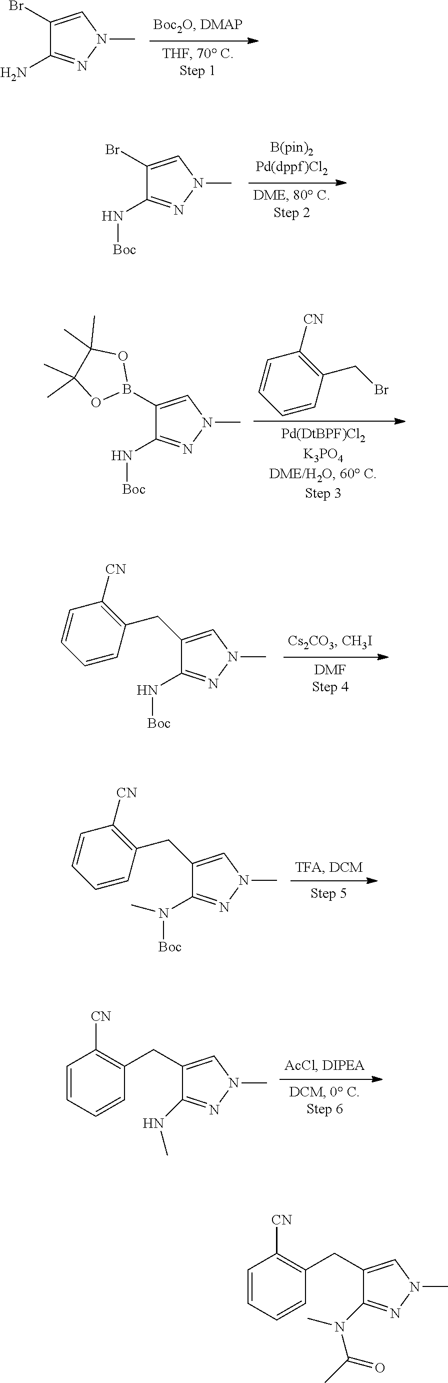

### Step 1: tert-butyl N-(4-bromo-1-methylpyrazol-3-yl)carbamate

To a stirred solution of 4-bromo-1-methylpyrazol-3-amine (0.500 g, 2.84 mmol) and di-tert-butyl dicarbonate (1.86 g, 8.52 mmol) in THF (5 mL) was added 4-Dimethylaminopyridine (0.035 g, 0.28 mmol) at rt. The resulting mixture was heated to 70° C. and stirred for 4 hours then cooled to rt and diluted with water (40 mL). Product was extracted with EtOAc and the organic layer was dried over Na₂SO₄ then concentrated in vacuo. The resulting material was dissolved in EtOH (10 mL) and to this was added NaOH (20% w/w in H2O, 2 mL) dropwise at room temperature. After stirring at rt for 2 h the mixture was diluted with water (40 mL) and extracted with EtOAc. The organic layer was dried over Na₂SO₄ and concentrated in vacuo. The crude material was purified by silica gel column chromatography (1:1 EtOAc/Pet. Ether) giving the desired product as an off-white solid (0.200 g, 25% yield)

ESI-MS m/ [symbol] : 276.1 [M+H]⁺

### Step 2 tert-butyl N-[1-methyl-4-(4,4,5,5-tetramethyl-1,3,2-dioxaborolan-2-yl)pyrazol-3-yl]carbamate

To a solution of (tert-butyl N-(4-bromo-1-methylpyrazol-3-yl)carbamate) (0.500 g, 1.81 mmol) and bis(pinacolato)diboron (0.690 g, 2.72 mmol) in DME (5 mL) was added potassium acetate (0.355 g, 3.62 mmol) and 1,1′-Bis(diphenylphosphino)ferrocene]dichloropalladium (132.5 mg, 0.18 mmol). The resulting solution was heated to 80° C. at stirred for 2 h then cooled to rt and concentrated in vacuo. The crude material was purified by C18 chromatography (10% to 90% MeCN/H₂O in 40 min) giving the product as an off-white solid (0.290 g, 49% Yield)

ESI-MS m/z 324.1 [M+H]⁺

### Step 3 tert-butyl (4-(2-cyanobenzyl)-1-methyl-1H-pyrazol-3-yl)carbamate

Into a 100 mL round-bottom flask was added (tert-butyl N-[1-methyl-4-(4,4,5,5-tetramethyl-1,3,2-dioxaborolan-2-yl)pyrazol-3-yl]carbamate) (4.5 g, 13.92 mmol), 1,1′-Bis (di-t-butylphosphino)ferrocene palladium dichloride (0.91 g, 1.39 mmol) and potassium phosphate tribasic (8.87 g, 41.77 mmol). This mixture was then dissolved in DME (10 mL) and H₂O (2 mL) and the resulting mixture was heated to 60° C. then stirred for 2 h. The mixture was allowed to cool down to room temperature, diluted with water and the product was extracted with EtOAc, dried over Na₂SO₄ and concentrated in vacuo. The residue was purified by silica gel column chromatography and eluted with EtOAc/petroleum to afford the desired product as a yellow oil (2.2 g, 48% yield)

ESI-MS m/z 313.1[M+H]⁺

### Step 4 tert-butyl N-{4-[(2-cyanophenyl)methyl]-1-methylpyrazol-3-yl}-N-methylcarbamate

Into a 40 mL vial was added (tert-butyl (4-(2-cyanobenzyl)-1-methyl-1H-pyrazol-3-yl)carbamate) (1 g, 3.20 mmol), cesium carbonate (2.61 g, 8.00 mmol), iodomethane (0.50 g, 3.52 mmol, 1.1 equiv) and DMF (10 mL). The resulting mixture was stirred for 3 h at rt then diluted with H2O and the product was extracted with EtOAc. The combined organic layers were washed with brine, dried over Na₂SO₄ and concentrated in vacuo. The crude residue was purified by silica gel column chromatography, eluted with EtOAc/Pet. Ether (1:2) giving the desired product as a light yellow oil (0.600 g, 57% yield)

ESI-MS m/z 271.0[M+H-^{t}Bu]⁺

### Step 5 2-{[1-methyl-3-(methylamino)pyrazol-4-yl]methyl}benzonitrile

To a stirred solution of (tert-butyl N-{4-[(2-cyanophenyl)methyl]-1-methylpyrazol-3-yl}-N-methylcarbamate) (350 mg, 1.07 mmol) in DCM (6 mL) was added trifluoroacetic acid (3 mL) portion wise at 0° C. The reaction mixture was stirred for 2 h at rt then concentrated in vacuo. The resulting material was diluted with H2O and to this was added NaHCO₃(Sat) until pH 9 was obtained, this mixture was then extracted with EtOAc and the organic layer was collected, dried over Na₂SO₄ then concentrated in vacuo giving the desired product as a light yellow solid (0.210 g, 86% yield).

ESI-MS m/z 227.0 [M+H]⁺

### Step 6 N-{4-[(2-cyanophenyl)methyl]-1-methylpyrazol-3-yl}-N-methylacetamide

To a stirred solution of 2-{[1-methyl-3-(methylamino)pyrazol-4-yl]methyl}benzonitrile (0.420 g, 1.86 mmol) and N,N diisopropyl ethyl amine (0.600 mg, 4.64 mmol) in DCM (5 mL) was added acetyl chloride (0.160 g, 2.04 mmol) dropwise at 0° C. The resulting mixture was stirred for 2 h at room temperature and then cooled to 0° C. and quenched with water. Product was extracted with EtOAc and the combined organic layers were dried over Na₂SO₄ then concentrated in vacuo. The crude product was purified by silica gel column chromatography, eluted with EtOAc/Pet. Ether (1:2) to afford the product as a light yellow solid (380 mg, 76% yield)

ESI-MS m/z 269.1[M+H]⁺

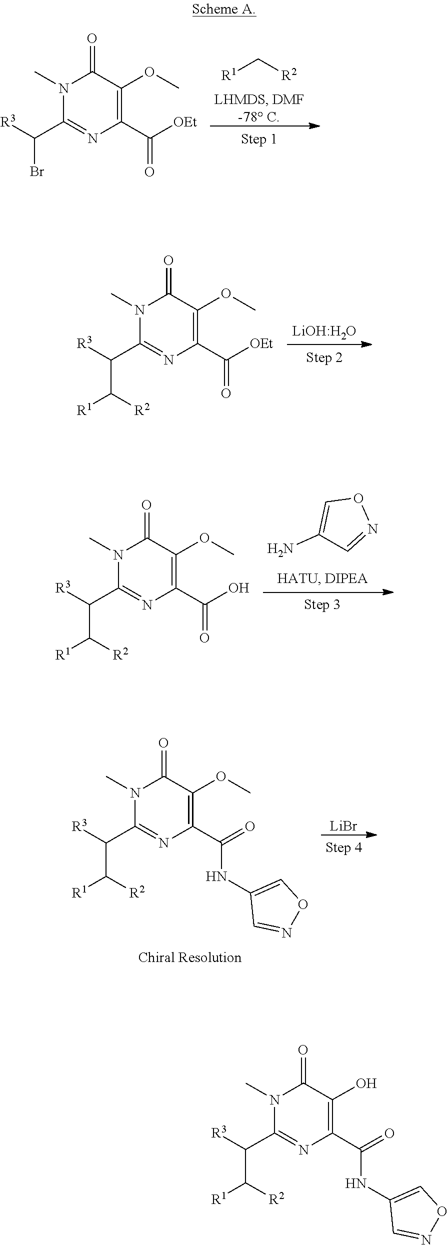

### Example 1

### Synthesis of 2-(1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide

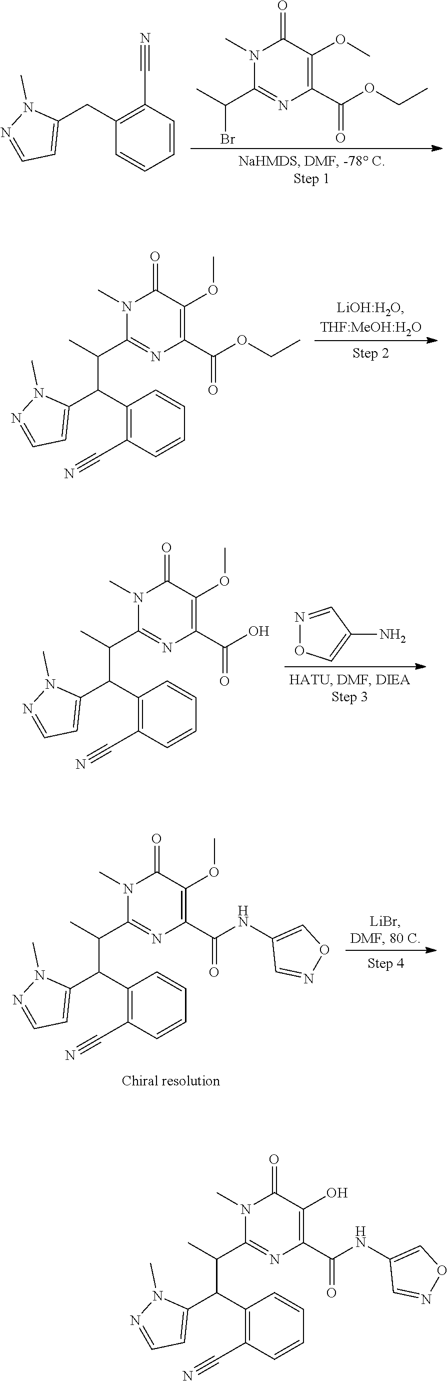

### Step 1: Ethyl 2-(1-(2-cyanophenyl)-1-(1-methyl-1H-pyrazol-5-yl)propan-2-yl)-5-methoxy-1-methyl-6-oxo-1,6-dihydropyrimidine-4-carboxylate

To a stirred solution of 1M NaHMDS in THF (7.8 ml, 7.8 mmol) was added 2-((1-methyl-1H-pyrazol-5-yl)methyl)benzonitrile (1.23 g, 6.3 mmol) in DMF (4 ml) dropwise at −78° C. for 15 minutes. Reaction mixture was stirred at −78° C. for 1 hour under an atmosphere of Nitrogen. Ethyl 2-(1-bromoethyl)-5-methoxy-1-methyl-6-oxo-1,6-dihydropyrimidine-4-carboxylate (1 g, 3.1 mmol) in DMF (6 ml) was added dropwise at −78° C. for 15 minutes. The reaction mixture was stirred for 30 minutes. After completion of reaction (confirmed by TLC), the reaction mixture was quenched with Saturated solution of aq. NH₄Cl (10 ml) and extracted with EtOAc (3×30 ml). The combined organic layer was washed with brine (30 ml), dried over anhydrous sodium sulphate, filtered, and concentrated under reduced pressure. The crude compound was purified by using Combi-flash chromatography to give pure title compound (1.17 g, 73%).

Isomer-1(D1)_LCMS: m/ [symbol] 436.6 [M+1].

Isomer-1(D2)_LCMS: m/ [symbol] 436.6 [M+1].

[symbol]

Step 2: 2-(1-(2-cyanophenyl)-1-(1-methyl-1H-pyrazol-5-yl)propan-2-yl)-5-methoxy-1-methyl-6-oxo-1,6-dihydropyrimidine-4-carboxylic acid:

To a stirred solution of Ethyl 2-(1-(2-cyanophenyl)-1-(1-methyl-1H-pyrazol-5-yl)propan-2-yl)-5-methoxy-1-methyl-6-oxo-1,6-dihydropyrimidine-4-carboxylate (1.17 g, 2.7 mmol) in methanol:THF:water (1:1:1, 35 ml) was added sodium hydroxide (0.128 g, 3.2 mmol) at room temperature. The reaction mixture was stirred at room temperature for 2 hours. After completion of the reaction (confirmed by TLC), the reaction mixture was concentrated under reduced pressure. The reaction mixture was diluted with water (15 ml) and extracted with EtOAc (3×15 ml) to remove impurities. The aqueous layer was acidified to pH: 2-3 with 1N HCl and extracted with EtOAc (3×15 ml). The combined organic layer was washed with brine (40 ml), dried over anhydrous sodium sulphate, filtered, and concentrated under reduced pressure to obtain crude title compound (0.980 g). The crude compound was used in the next step without further purification.

Isomer-1(D1)_LCMS: m/ [symbol] 408.2 [M⁺+1].

Isomer-1(D2)_LCMS: m/ [symbol] 408.2 [M⁺+1].

### Step 3: 2-(1-(2-cyanophenyl)-1-(1-methyl-1H-pyrazol-5-yl)propan-2-yl)-N-(isoxazol-4-yl)-5-methoxy-1-methyl-6-oxo-1,6-dihydropyrimidine-4-carboxamide

To a stirred solution of 2-(1-(2-Cyanophenyl)-1-(1-methyl-1H-pyrazol-5-yl)propan-2-yl)-5-methoxy-1-methyl-6-oxo-1,6-dihydropyrimidine-4-carboxylic acid (0.980 g, 2.4 mmol) in DMF (5 ml) was added HATU (1.36 g, 3.6 mmol) and Isoxazol-4-amine (0.262 g, 3.1 mmol) at room temperature. The reaction mixture was stirred at room temperature for 1 hour. DIPEA (1.25 ml, 7.2 mmol) was added to it and the reaction mixture was stirred for another 1 hour. After completion of reaction (monitor by TLC), the reaction mixture was diluted with water (40 ml) and aqueous layer was extracted with EtOAc (3×30 ml). The combined organic layer was washed with brine (50 ml), dried over anhydrous sodium sulphate, filtered, and concentrated under reduced pressure. The crude compound was purified by Combi-flash column chromatography to give pure title compound (0.6 g, 53%). The diastereomer mixture (600 mg) was separated by using Prep HPLC to obtain two separated diastereomers as D1 (220 mg) and D2 (240 mg).

Isomer-1 (D1)_LCMS: m/ [symbol] 474.3 [M⁺+1].

Isomer-1 (D2)_LCMS: m/ [symbol] 474.3 [M⁺+1].

Isomer-1 (D1): ¹H NMR (400 MHz, DMSO-d6): δ 1.31 (d, J=6.4 Hz, 3H), 3.61 (s, 3H), 3.74 (s, 3H), 4.01 (s, 3H), 4.16-4.23 (m, 1H), 5.16 (d, J=10.8 Hz, 1H), 6.66 (d, J=0.8 Hz, 1H), 7.30 (t, J=7.6 Hz, 1H), 7.42 (d, J=1.2 Hz, 1H), 7.59-7.64 (m, 2H), 7.87 (d, J=8.0 Hz, 1H), 8.77 (s, 1H), 9.28 (s, 1H), 10.49 (s, 1H).

Isomer-1 (D2): ¹H NMR (400 MHz, DMSO-d6): δ 1.32 (d, J=6.4 Hz, 3H), 3.34 (s, 3H), 3.59 (s, 3H), 4.00 (s, 3H), 4.16-4.20 (m, 1H), 5.14 (d, J=10.8 Hz, 1H), 6.66 (d, J=1.6 Hz, 1H), 7.30 (t, J=8.0 Hz, 1H), 7.42 (bs, 1H), 7.58-7.63 (m, 2H), 7.86 (d, J=8.0 Hz, 1H), 8.76 (s, 1H), 9.27 (s, 1H), 10.47 (s, 1H).

### Chiral HPLC Method:

The diastereomers of title compound was resolved by Chiral SFC [D1: (CHIRALPAK IC(250*21) mm, 5 u; MeOH:IPA (50:50) in Hexanes+0.1% DEA)] and [D2: (CHIRALPAK IC(250*21) mm, 5 u; MeOH:IPA (50:50) in Hexanes+0.1% DEA)] to furnish the enantiopure compounds.

Chiral HPLC: FR-1 (Isomer-1; D1E1): RT=12.45; FR-2 (Isomer-2; D1E2) RT=14.04; FR-3 (Isomer-3; D2E1): RT=4.02; FR-4 (Isomer-4; D2E2): RT=4.13.

### Step 4: 2-(1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide

To a solution of 2-(1-(2-Cyanophenyl)-1-(1-methyl-1H-pyrazol-5-yl)propan-2-yl)-N-(isoxazol-4-yl)-5-methoxy-1-methyl-6-oxo-1,6-dihydropyrimidine-4-carboxamide (0.030 g, 0.06 mmol) in DMF (0.3 ml), Lithium bromide (0.055 g, 0.6 mmol) was added at room temperature. The reaction mixture was heated and stirred at 130° C. for 1 hour under microwave irradiation. After completion of reaction (confirmed by TLC), the reaction mixture was loaded on RP Gold column and purified using acetonitrile and 0.1% formic acid in water to give pure title compound (0.006 g, 20%).

Isomer-1 (D1E1) LCMS: m/ [symbol] 458.3 [M⁺−1].

Isomer-2 (D1E2) LCMS: m/ [symbol] 460.4 [M⁺+1].

Isomer-3 (D2E1) LCMS: m/ [symbol] 458.3 [M⁺−1].

Isomer-4 (D2E2) LCMS: m/ [symbol] 458.4 [M⁺−1].

Isomer-1_D1E1: ¹H NMR (400 MHz, DMSO-d6): δ 1.35 (d, J=6.0 Hz, 3H), 3.54 (s, 3H), 4.01 (s, 3H), 4.12-4.16 (m, 1H), 5.21 (d, J=10.8 Hz, 1H), 6.71 (s, 1H), 7.28 (t, J=7.2 Hz, 1H), 7.43 (s, 1H) 7.58-7.64 (m, 2H), 7.86 (d, J=8.0 Hz, 1H), 8.85 (s, 1H), 9.31 (s, 1H), 10.67 (s, 1H), 11.27 (s, 1H).

Isomer-2_D1E2: ¹H NMR (400 MHz, DMSO-d6): δ 1.35 (d, J=6.4 Hz, 3H), 3.54 (s, 3H), 4.01 (s, 3H), 4.12-4.16 (m, 1H), 5.21 (d, J=10.8 Hz, 1H), 6.71 (s, 1H), 7.28 (t, J=7.6 Hz, 1H), 7.43 (d, J=1.2 Hz, 1H) 7.58-7.63 (m, 2H), 7.86 (d, J=8.0 Hz, 1H), 8.85 (s, 1H), 9.30 (s, 1H), 10.68 (s, 1H), 11.27 (s, 1H).

Isomer-3_D2E1: ¹H NMR (400 MHz, DMSO-d6): δ 1.13 (d, J=6.0 Hz, 3H), 3.64 (s, 3H), 3.77 (s, 3H), 4.11-4.16 (m, 1H), 5.22 (d, J=10.8 Hz, 1H), 6.45 (s, 1H), 7.20 (s, 1H), 7.52 (t, J=7.6 Hz, 1H), 7.79 (t, J=7.6 Hz, 1H), 7.90 (d, J=7.6 Hz, 1H), 7.97 (d, J=8.0 Hz, 1H), 8.82 (s, 1H), 9.30 (s, 1H), 10.66 (s, 1H), 11.14 (s, 1H).

Isomer-4_D2E2: ¹H NMR (400 MHz, DMSO-d6): δ 1.15 (d, J=6.4 Hz, 3H), 3.65 (s, 3H), 3.74 (s, 3H), 4.11-4.16 (m, 1H), 5.22 (d, J=10.8 Hz, 1H), 6.45 (s, 1H), 7.21 (d, J=1.2 Hz, 1H), 7.52 (t, J=7.6 Hz, 1H), 7.80 (t, J=7.6 Hz, 1H), 7.91 (d, J=7.6 Hz, 1H), 7.96 (d, J=8.0 Hz, 1H), 8.83 (s, 1H), 9.32 (s, 1H), 10.47 (s, 1H), 11.13 (s, 1H).

HPLC: FR-1 (Isomer-1; D1E1): R_{T}=4.45 (97%); FR-2 (Isomer-2; D1E2): R_{T}=4.46 (97%); FR-3 (Isomer-3; D2E1): R_{T}=4.54 (98%); FR-4 (Isomer-4; D2E2): R_{T}=4.55 (96%).

The following compounds in Table 2 were prepared according to the methods described in Scheme A.

TABLE 2

| ID | Structure | ¹H NMR | LCMS |
|---|---|---|---|
| Example 2 | 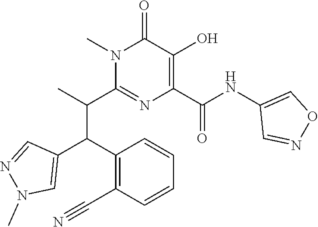 [SMILES: CC(c1nc(C(=O)Nc2cnoc2)c(O)c(=O)n1C)C(c1cnn(C)c1)c1ccccc1C#N] | Isomer-1_ D1E1: ¹H NMR (400 MHz, DMSO-d₆): δ 1.27 (d, J = 4.8 Hz, 3H), 3.59 (s, 3H), 3.80 (s, 3H), 3.93-4.08 (m, 1H), 4.92 (d, J = 10.8 Hz, 1H), 7.18 (t, J = 7.2 Hz, 1H), 7.51-7.60 (m, 3H), 7.75(bs, 2H), 8.83 (s, 1H), 9.23 (s, 1H), 11.04 (s, 1H). Isomer-2_ D1E2: ¹H NMR (400 MHz, DMSO-d₆): δ 1.33 (d, J = 5.6 Hz, 3H), 3.60 (s, 3H), 3.81 (s, 3H), 4.05-4.13 (m, 1H), 4.98 (d, J = 10.8 | Isomer-1_(D1E1) CMS: m/z 460.32 [M⁺ + 1]. Isomer-2_(D1E2) LCMS: m/z 460.62 [M⁺ + 1]. Isomer-3_(D2E1) _LCMS: m/z 460.63 [M⁺ + 1]. Isomer- 4_(D2E2) LCMS: m/z 460.31 [M⁺ + 1]. HPLC: FR-1 (Isomer-1; D1E1): R_{T} = 4.43 (96%); FR-2 (Isomer-2; D1E2): R_{T} = 4.43 100%); FR-3 (Isomer-3; D2E1): R_{T} = 4.53 100%); FR-4 (Isomer-4; D2E2): R_{T} = 4.86 (97%). |
| | | Hz, 1H), 7.20 (t, J = 7.2 Hz, | |
| | | 1H), 7.55-7.59 (m, 3H), 7.78 | |
| | | (bs, 2H), 8.87 (s, 1H), 9.30 | |
| | | (s, 1H), 10.50 (s, 1H), 11.19 | |
| | | (s, 1H). | |
| | | Isomer-3_ D2E1: ¹H NMR | |
| | | (400 MHz, DMSO-d₆): δ 1.14 | |
| | | (d, J = 6.4 Hz, 3H), 3.49 (s, | |
| | | 3H), 3.65 (s, 3H), 4.02-4.09 | |
| | | (m, 1H), 4.87 (d, J = 10.4 | |
| | | Hz, 1H), 7.12 (s, 1H), 7.39 (s, | |
| | | 1H), 7.47 (t, J = 7.6 Hz, 1H), | |
| | | 7.79 (t, J = 7.6 Hz 1H), 7.83 | |
| | | (d, J = 7.6 Hz, 1H), 7.93 (s, | |
| | | J = 8.0 Hz, 1H), 8.87 (s, 1H), | |
| | | 9.33 (s, 1H), (s, 10.49 (s, | |
| | | 1H), 11.21 (s, 1H). | |
| | | Isomer-4_ D2E2: ¹H NMR | |
| | | (400 MHz, DMSO-d₆): δ 1.14 | |
| | | (d, J = 6.4 Hz, 3H), 3.49 (s, | |
| | | 3H), 3.65 (s, 3H), 4.02-4.07 | |
| | | (m, 1H), 4.87 (d, J = 10.4 Hz, | |
| | | 1H), 7.12 (s, 1H), 7.39 (s, | |
| | | 1H), 7.47 (t, J = 7.6 Hz, 1H), | |
| | | 7.79 (t, J = 7.6 Hz, 1H), 7.83 | |
| | | (d, J = 7.6 Hz, 1H), 7.93 (d, | |
| | | J = 7.6 Hz, 1H), 8.87 (s, 1H), | |
| | | 9.33 (s, 1H), 10.45 (s, 1H), | |
| | | 11.21 (s, 1H). | |
| Example 3 | 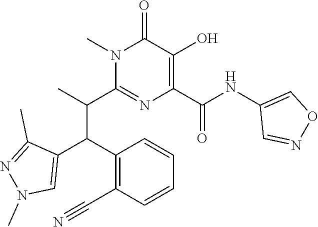 [SMILES: Cc1nn(C)cc1C(c1ccccc1C#N)C(C)c1nc(C(=O)Nc2cnoc2)c(O)c(=O)n1C] | Isomer-1_ D1E1: ¹H NMR (400 MHz, DMSO-d₆): δ 1.37 (d, J = 6.4 Hz, 3H), 2.27 (s, 3H), 3.57 (s, 3H), 3.75 (s, 3H), 3.99-4.04 (m, 1H), 4.85 (d, J = 10.8 Hz, 1H), 7.21 (t, J = 7.6 Hz, 1H), 7.56-7.59 (m, 2H), 7.74 (d, J = 8.4 Hz, 1H), 7.92 (s, 1H), 8.87 (s, 1H), 9.31 (s, 1H), 10.45 (s, 1H), 11.23 (s, 1H). Isomer-2_ D1E2: ¹H NMR (400 MHz, DMSO-d₆): δ 1.35 (d, J = 6.4 Hz, 3H), 2.26 (s, | Isomer-1_(D1E1)_LCMS: m/z 474.0 [M⁺ + 1].Isomer 2 (D1E2)_LCMS: m/z 74.4 [M⁺ + 1].Isomer-3_(D2E1)_LCMS: m/z 474.1 [M⁺ + 1]. Isomer- 4_(D2E2)_LCMS: m/z 474.0 [M⁺ + 1]. HPLC: FR-1 (Isomer-1; D1E1, 105 mg): R = 4.43 100%); FR-2 (Isomer-2; D1E2, 118 mg): R_{T} = 4.43 (96%); FR-3 (Isomer-3; D2E1, 31 mg): R_{T} = 4.55 (94%); FR-4 (Isomer-4; D2E2, 33 mg): R_{T} = 4.55 (94%). |
| | | 3H), 3.56 (s, 3H), 3.74 | |
| | | (s, 3H), 3.97-4.02 (m, 1H), | |
| | | 4.84 (d, J = 10.8 Hz, 1H), 7.20 | |
| | | (t, J = 7.2 Hz, 1H), 7.53-7.58 | |
| | | (m, 2H), 7.73 (d, J = 7.6 | |
| | | Hz, 1H), 7.89 (s, 1H), | |
| | | 8.85 (s, 1H), 9.30 (s, 1H), | |
| | | 10.50 (s, 1H), 11.23 (s, 1H). | |
| | | Isomer-3_ D2E1: ¹H NMR | |
| | | (400 MHz, DMSO-d₆): δ 1.15 | |
| | | (d, J = 6.4 Hz, 3H), 1.91 (s, | |
| | | 3H), 3.54 (s, 3H), 3.65 (s, | |
| | | 3H), 4.02-4.06 (m, 1H), 4.83 | |
| | | (d, J = 10.4 Hz, 1H), 7.45 (t, | |
| | | J = 7.2 Hz, 1H), 7.64 (s, 1H), | |
| | | 7.75-7.83 (m, 2H), 7.94 (d, | |
| | | J = 8.0 Hz, 1H), 8.84 (s, 1H), | |
| | | 9.32 (s, 1H), 10.60 (s, 1H), | |
| | | 11.11 (s, 1H). | |
| | | Isomer-4_ D2E2: ¹H NMR | |
| | | (400 MHz, DMSO-d₆): δ 1.16 | |
| | | (d, J = 6.4 Hz, 3H), 1.92 (s, | |
| | | 3H), 3.55 (s, 3H), 3.61 (s, | |
| | | 3H), 4.02-4.06 (m, 1H), 4.84 | |
| | | (d, J = 10.8 Hz, 1H), 7.46 (t, | |
| | | J = 7.6 Hz, 1H), 7.65 (s, 1H), | |
| | | 7.76-7.84 (m, 2H), 7.95(d, | |
| | | J = 8.0 Hz, 1H), 8.85(s, 1H), | |
| | | 9.33 (s, 1H), 10.61 (s, 1H), | |
| | | 11.12 (s, 1H). | |
| Example 4 | 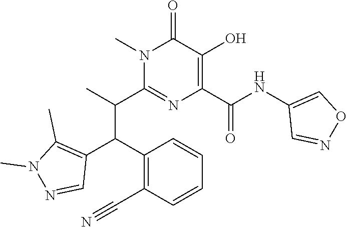 [SMILES: Cc1c(C(c2ccccc2C#N)C(C)c2nc(C(=O)Nc3cnoc3)c(O)c(=O)n2C)cnn1C] | Isomer-1_ D1E1: ¹H NMR (400 MHz, DMSO-d₆): δ 1.31 (d, J = 6.4 Hz, 3H), 2.42 (s, 3H), 3.60 (s, 3H), 3.71 (s, 3H), 4.06-4.10 (m, 1H), 4.89 (d, J = 11.2 Hz, 1H), 7.20 (t, J = 7.6 Hz, 1H), 7.53-7.58 (m, 2H), 7.71 (s, 1H), 7.83 (d, J = 8.0 Hz, 1H), 8.87 (s, 1H), 9.31 (s, 1H), 10.55 (s, 1H), 11.24 (s, 1H). Isomer-2_D1E2: ¹H NMR (400 MHz, DMSO-d₆): δ 1.31 | Isomer-1_(D1E1) LCMS: m/z 474.6 [M⁺ + 1]. Isomer-2_(D1E2) LCMS: m/z 474.5 [M⁺ + 1]. Isomer-3_(D2E1) _LCMS m/z 474.0: [M⁺ + 1]. Isomer- 4_(D2E2) LCMS: m/z 474.1 [M⁺ + 1]. HPLC: FR-1 (Isomer-1; D1E1, 102 mg): R_{T} = 4.38 (99%); FR-2 (Isomer-2; D1E2, 145 mg): R_{T} = 4.37(99%); FR-3 (Isomer-3; D2E1, 57mg): R_{T} = 4.46 (98%); FR-4 (Isomer-4; D2E2, 70mg): R_{T} = 4.48 100%). |
| | | (d, J = 6.4 Hz, 3H), 2.42 (s, | |
| | | 3H), 3.61 (s, 3H), 3.71 (s, | |
| | | 3H), 4.07-4.11 (m, 1H), 4.89 | |
| | | (d, J = 10.8 Hz, 1H), 7.20 (t, | |
| | | J = 7.6 Hz, 1H), 7.54-7.58 (m, | |
| | | 2H), 7.72 (s, 1H), | |
| | | 7.84 (d, J = 7.6 Hz, 1H), | |
| | | 8.87 (s, 1H), 9.32 (s, 1H), | |
| | | 10.46 (s, 1H), 11.21 (s, | |
| | | 1H). | |
| | | Isomer-3_ D2E1: ¹H NMR | |
| | | (400 MHz, DMSO- d₆): δ 1.19 | |
| | | (d, J = 5.6 Hz, 3H), 2.05 (s, | |
| | | 3H), 3.51 (s, 3H), 3.55 (s, | |
| | | 3H), 4.10-4.20 (m, 1H), 4.84 | |
| | | (d, J = 10.8 Hz, 1H), 7.45 (s, | |
| | | 2H), 7.77-7.83 (m, 2H), 8.07 | |
| | | (d, J = 7.6 Hz, 1H), 8.87 (s, | |
| | | 1H), 9.34 (s, 1H), 10.65 (s, | |
| | | 1H), 11.10 (s, 1H). | |
| | | Isomer-4_ D2E2: ¹H NMR | |
| | | (400 MHz, DMSO-d₆): δ 1.19 | |
| | | (d, J = 6.4 Hz, 3H), 2.06 (s, | |
| | | 3H), 3.52 (s, 3H), 3.55 (s, | |
| | | 3H), 4.12-4.17 (m, 1H), 4.84 | |
| | | (d, J = 10.8 Hz, 1H), | |
| | | 7.43-7.47 (m, 2H), 7.77-7.83 | |
| | | (m, 2H), 8.08 (d, J = 8.0 Hz, | |
| | | 1H), 8.87 (s, 1H), 9.34 (s, | |
| | | 1H), 10.76 (s, 1H), 11.10 (s, | |
| | | 1H). | |
| Example 5 | 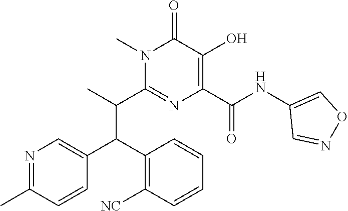 [SMILES: Cc1ccc(C(c2ccccc2C#N)C(C)c2nc(C(=O)Nc3cnoc3)c(O)c(=O)n2C)cn1] | Isomer-1_ D1E1: ¹H NMR (300 MHz, DMSO-d₆): δ 9.29 (s, 1H), 8.85 (s, 1H), 8.72 (s, 1H), 7.96-7.86 (m, 2H), 7.64-7.56 (m, 2H), 7.29-7.19 (m, 2H), 5.16 (d, 1H), 4.27-4.23 (m, 1H), 3.66 (s, 3H), 3.28 (s, 3H), 1.17 (d, 3H) Isomer-2_ D1E2: ¹H NMR (300 MHz, DMSO-d₆): δ 11.26 (brs, 1H), 10.72 (brs, 1H), 9.29 (s, 1H), 8.87 (s, 1H), 8.74 (s, 1H), 7.99 (d, | Isomer-1_D1E1: m/z 471.2 [M⁺ + H] >98% ee Isomer-2_D1E2: m/z 471.1 [M⁺ + H] >97% ee Isomer-3_D2E1: m/z 471.1 [M⁺ + H] >98% ee Isomer-4_D2E2: m/z 471.2 [M⁺ + H] >98% ee |
| | | J = 8.1 Hz, 1H), 7.90 (d, | |
| | | J = 8.0 Hz, 1H), 7.66-7.53 (m, | |
| | | 2H), 7.33-7.19 (m, 2H), 5.21 | |
| | | (d, J = 11.1 Hz, 1H), 4.31 (s, | |
| | | 1H), 3.67 (s, 3H), 2.45(s, 3H), | |
| | | 1.21 (d, J = 6.5 Hz, 3H). | |
| | | Isomer-3_ D2E1: ¹H NMR | |
| | | (300 MHz, DMSO-d₆): δ | |
| | | 11.15 (brs, 1H), 10.84 (brs, | |
| | | 1H), 9.33 (s, 1H), 8.89 (s, | |
| | | 1H), 8.33 (s, 1H), 8.19 (d, | |
| | | J = 8.0 Hz, 1H), 7.85-7.79 (m, | |
| | | 2H), 7.64-7.54 (m, 1H), 7.48 | |
| | | (t, J = 7.6 Hz, 1H), 7.06(d, | |
| | | J = 8.1 Hz, 1H), 5.04 (d, | |
| | | J = 11.0 Hz, 1H), 4.34-4.28 | |
| | | (m, 1H), 3.51 (s, 3H), 2.29 (s, | |
| | | 3H), 1.40-1.21 (d, 3H), | |
| | | 1.40-1.21 (3H | |
| | | Isomer-4_ D2E2: ¹H NMR | |
| | | (300 MHz, DMSO-d₆): δ | |
| | | 11.14 (brs, 1H), 10.85 (s, 1H), | |
| | | 9.33 (s, 1H), 8.90 (s, 1H), 8.33 | |
| | | (d, J = 2.3 Hz, 1H), 8.19 (d, | |
| | | J= 7.9 Hz, 1H), 7.85-7.80 (m, | |
| | | 2H), 7.59 (dd, J = 8.1, 2.4 Hz, | |
| | | 1H), 7.51-7.45 (m, 1H), 7.06 | |
| | | (d, J = 8.0 Hz, 1H), 5.04 (d, | |
| | | J = 10.9 Hz, 1H), 4.42-4.19 | |
| | | (m, 1H), 3.51 (s, 3H), 2.29 (s, | |
| | | 3H), 1.29 (d, J = 6.4 Hz, 3H). | |
| Example 6 | 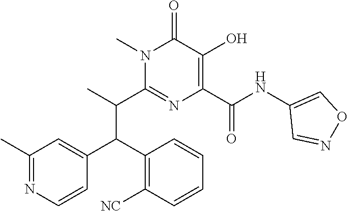 [SMILES: Cc1cc(C(c2ccccc2C#N)C(C)c2nc(C(=O)Nc3cnoc3)c(O)c(=O)n2C)ccn1] | Isomer-1_ D1E1: ¹H NMR (400 MHz, DMSO-d₆): δ 11.13 (s, 1H), 10.50 (s, 1H), 9.30 (s, 1H), 8.88 (s, 1H), 8.45 (d, 1H), 7.91 (d, 1H), 7.63-7.61 (m, 3H), 7.59-7.58 (m, 1H), 7.27-7.23 (m, 1H), 5.18 (d, 1H), 4.35-4.32 (m, 1H), 3.67 (s, 3H), 2.47 (s, 3H), 1.24 (d, 3H) Isomer-2_ D1E2: ¹H NMR (400 MHz, DMSO-d₆): δ 11.13 (s, 1H), 10.50 (s, 1H), | Isomer-1_D1E1: m/z 471.3 [M + H] >98% ee Isomer-2_D1E2: m/z 471.2 [M⁺ + H] >96% ee Isomer-3_D2E1: m/z 471.1 [M⁺ + H] >95% ee Isomer-4_D2E2: m/z 471.2 [M⁺ + H] >98% ee |
| | | 9.30 (s, 1H), 8.88 (s, 1H), 8.45 | |
| | | (d, 1H), 7.91 (d, 1H), | |
| | | 7.63-7.61 (m, 3H), 7.59-7.58 | |
| | | (m, 1H), 7.27-7.23 (m, 1H), | |
| | | 5.18 (d, 1H), 4.35-4.29 (m, | |
| | | 1H), 3.67 (s, 3H), 2.47 (s, | |
| | | 3H), 1.24 (d, 3H) | |
| | | Isomer-3_ D2E1: ¹H NMR | |
| | | (400 MHz, DMSO-d₆): δ | |
| | | 11.15 (s, 1H), 10.48 (s, 1H), | |
| | | 9.34 (s, 1H), 8.90 (s, 1H), 8.22 | |
| | | (d, 1H), 8.14 (d, 1H), | |
| | | 7.87-7.80 (m, 2H), 7.52-7.48 | |
| | | (m, 1H), 7.17 (s, 1H), 7.09 (d, | |
| | | 1H), 5.02 (d, 1H), 4.32-4.17 | |
| | | (m, 1H), 3.55 (s, 3H), 2.28 (s, | |
| | | 3H), 1.26 (d, 3H). | |
| | | Isomer-4_ D2E2: ¹H NMR | |
| | | (400 MHz, DMSO-d₆): δ | |
| | | 11.17 (s, 1H), 10.48 (s, 1H), | |
| | | 9.34 (s, 1H), 8.90 (s, 1H), 8.27 | |
| | | (d, 1H), 8.14 (d, 1H), 7.88- | |
| | | 7.81 (m, 2H), 7.53-7.49 (m, | |
| | | 1H), 7.30-7.21 (m, 2H), 5.07 | |
| | | (d, 1H), 4.33-4.28 (m, 1H), | |
| | | 3.56 (s, 3H), 2.32 (s, 3H), 1.26 | |
| | | (d, 3H). | |
| Example 7 | 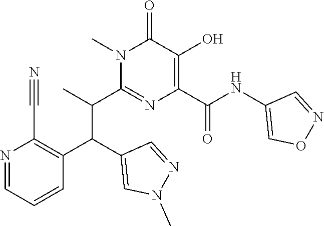 [SMILES: CC(c1nc(C(=O)Nc2cnoc2)c(O)c(=O)n1C)C(c1cnn(C)c1)c1cccnc1C#N] | Isomer-1_ D1E1: ¹H NMR (400 MHz, DMSO-d₆): δ 1.35 (d, J = 6.4 Hz, 3H), 3.61 (s, 3H), 3.81 (s, 3H), 4.05-4.10 (m, 1H), 5.04 (d, J = 10.8 Hz, 1H), 7.58-7.62 (m, 2H), 7.81 (s, 1H), 8.28 (d, J = 8.0 Hz, 1H), 8.40 (d, J = 4.0 Hz, 1H), 8.85 (s, 1H), 9.29 (s, 1H), 10.56 (bs, 1H), 11.16 (s, 1H). Isomer-2_ D1E2: ¹H NMR (400 MHz, DMSO-d₆): δ 1.35 (d, J = 6.4 Hz, 3H), 3.61 (s, | Isomer-1_ (D1E1) LCMS: m/z 461.2 [M⁺+ 1]. Isomer-2_ (D1E2) LCMS: m/z 461.2 [M⁺ + 1]. Isomer-3_(D2E1) LCMS: m/z 461.2 [M⁺ + 1]. Isomer-4_(D2E2) LCMS:m/z 461.2 [M⁺ + 1]. HPLC: FR-1 (Isomer-1; D1E1): RT = 4.08 100%); FR- 2 (Isomer-2; D1E2): R_{T} = 4.08 (99%); FR- 3 (Isomer-3; D2E1): R_{T} = 4.12 100%); FR- 4 (Isomer-4; D2E2): R_{T} = 4.11 100%). |
| | | 3H), 3.81 (s, 3H), 4.05-4.09 | |
| | | (m, 1H), 5.04 (d, J = 11.2 Hz, | |
| | | 1H), 7.58-7.62 (m, 2H), 7.81 | |
| | | (s, 1H), 8.28 (d, J = 8.0 Hz, | |
| | | 1H), 8.40 (d, J = 4.0 Hz, 1H), | |
| | | 8.85 (s, 1H), 9.29 (s, 1H), | |
| | | 10.57 (bs, 1H), 11.16 (s, 1H). | |
| | | Isomer-3_ D2E1: ¹H NMR | |
| | | (400 MHz, DMSO-d₆): δ 1.17 | |
| | | (d, J = 6.4 Hz, 3H), 3.51 (s, | |
| | | 3H), 3.66 (s, 3H), 4.05-4.10 | |
| | | (m, 1H), 4.89 (d, J = 10.0 Hz, | |
| | | 1H), 7.18 (s, 1H), 7.45 (s, | |
| | | 1H), 7.82-7.85 (m, 1H), 8.43 | |
| | | (d, J = 8.0 Hz, 1H), 8.66 (d, | |
| | | J = 4.4 Hz, 1H), 8.86 (s, 1H), | |
| | | 9.33 (s, 1H), 10.40 (bs, 1H), | |
| | | 11.23 (s, 1H). | |
| | | Isomer-4_D2E2: ¹H NMR | |
| | | (400 MHz, DMSO-d₆): ): δ | |
| | | 1.16 (d, J = 6.0 Hz, 3H), 3.51 | |
| | | (s, 3H), 3.66 (s, 3H), | |
| | | 4.03-4.09 (m, 1H), 4.89 (d, | |
| | | J = 10.0 Hz, 1H), 7.18 (s, 1H), | |
| | | 7.45 (s, 1H), 7.82-7.85 (m, | |
| | | 1H), 8.43 (d, J = 7.6 Hz, 1H), | |
| | | 8.66 (d, J = 4.0 Hz, 1H), 8.85 | |
| | | (s, 1H), 9.32 (s, 1H), 10.41 | |
| | | (bs, 1H), 11.23 (s, 1H). | |
| Example 8 | 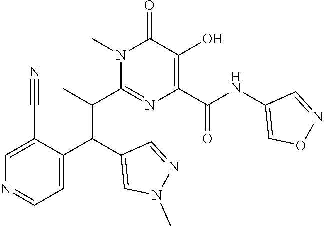 [SMILES: CC(c1nc(C(=O)Nc2cnoc2)c(O)c(=O)n1C)C(c1cnn(C)c1)c1ccncc1C#N] | Isomer-1_ D1E1: ¹H NMR (400 MHz, DMSO-d₆): δ 1.31 (d, J = 6.4 Hz, 3H), 3.64 (s, 3H), 3.81 (s, 3H), 4.09-4.13 (m, 1H), 4.52 (d, J = 10.8 Hz, 1H), 7.61 (s, 1H), 7.81 (d, J = 5.6 Hz, 2H), 8.64 (d, J = 4.8 Hz, 1H), 8.76 (s, 1H), 8.85 (s, 1H), 9.29 (s, 1H), 10.52 (s, 1H), 11.15 (s, 1H). Isomer-2_ D1E2: ¹H NMR (400 MHz, DMSO-d₆): δ 1.32 (d, J = 5.2 Hz, 3H), 3.65 (s, | Isomer-1_(D1E1) LCMS: m/z 461.3 [M⁺ + 1]. Isomer-2_ (D1E2) LCMS m/z 461.3 [M⁺ + 1]. Isomer-3_(D2E1) _LCMS: m/z 461.2 [M⁺ + 1]. Isomer-4_(D2E2) LCMS: m/z 461.2 [M⁺ + 1]. HPLC: FR-1 (Isomer-1; D1E1, 32 mg): R_{T} = 3.96 (99.49%); FR-2 (Isomer-2; D1E2, 30 mg): R_{T} = 3.95 (99.92%); FR-3 (Isomer-3; D2E1, 28 mg): R_{T} = 3.97 (99.41%); FR-4 (Isomer-4; D2E2, 27 mg): R_{T} = 3.97 (99.82%). |
| | | 3H), 3.81 (s, 3H), 4.11 (s, | |
| | | 1H), 5.03 (d, J = 10.8 Hz, 1H), | |
| | | 7.61 (s, 1H), 7.81 (s, 2H), 8.64 | |
| | | (d, J = 2.8 Hz, 1H), 8.76 (s, | |
| | | 1H), 8.86 (s, 1H), 9.29 (s, | |
| | | 1H), 10.49 (s, 1H), 11.15 (s, | |
| | | 1H). | |
| | | Isomer-3_ D2E1: ¹H NMR | |
| | | (400 MHz, DMSO-d₆): δ 1.19 | |
| | | (d, J = 6.0 Hz, 3H), 3.50 (s, | |
| | | 3H), 3.65 (s, 3H), 4.08-4.12 | |
| | | (m, 1H), 4.83 (d, J = 10.0 Hz, | |
| | | 1H), 7.15 (s, 1H), 7.44 (s, | |
| | | 1H), 8.03 (d, J = 5.2 Hz, 1H), | |
| | | 8.86-8.91 (m, 2H), 9.00 (s, | |
| | | 1H), 9.33 (s, 1H), 10.48 (s, | |
| | | 1H), 11.28 (s, 1H). | |
| | | Isomer-4_D2E2: ¹H NMR | |
| | | (400 MHz, DMSO-d₆): δ 1.19 | |
| | | (d, J = 6.4 Hz, 3H), 3.50 (s, | |
| | | 3H), 3.65 (s, 3H), 4.08-4.12 | |
| | | (m, 1H), 4.84 (d, J = 10.4 Hz, | |
| | | 1H), 7.15 (s, 1H), 7.44 (s, | |
| | | 1H), 8.03 (d, J = 5.2 Hz, 1H), | |
| | | 8.86-8.91 (m, 2H), 9.00 (s, | |
| | | 1H), 9.33 (s, 1H), 10.51 (s, | |
| | | 1H), 11.28 (s, 1H). | |
| Example 46 | 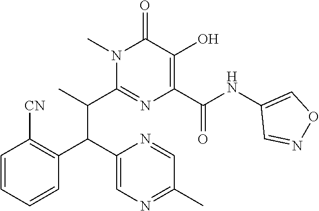 [SMILES: Cc1cnc(C(c2ccccc2C#N)C(C)c2nc(C(=O)Nc3cnoc3)c(O)c(=O)n2C)cn1] | Isomer-1_ D1E1 ¹H NMR (300 MHz, methanol-d₄) δ 9.26 (s, 1H), 8.88 (s, 1H), 8.74 (d, 1H), 8.64 (s, 1H), 7.96-7.93 (m, 1H), 7.58-7.56 (m, 2H), 7.28 (m, 1H), 5.52 (d, 1H), 4.58-4.48 (m, 1H), 3.74 (s, 3H), 2.57 (s, 3H), 1.30 (d, 3H). Isomer-2_D1E2 ¹H NMR 300 MHz, methanol-d₄) δ 9.26 (s, 1H), 8.88 (s, 1H), 8.74 (d, 1H), 8.64 (s, 1H), 7.95-7.93 | Isomer-1_D1E1: m/z 472.2 [M + H]>98% ee Isomer-2_D1E2: m/z 472.2 [M + H] >98% ee Isomer-3_D2E1: m/z 472.2 [M + H] >96% ee Isomer-4_D2E2: m/z 472.2 [M + H] >99% ee |
| | | (m, 1H), 7.61-7.56 (m, 2H), | |
| | | 7.30-7.27 (m, 1H), 5.51 (d, | |
| | | 1H), 4.58-4.48 (m, 1H), 3.74 | |
| | | (s, 3H), 2.57 (s, 3H), 1.30 (d, | |
| | | 3H). | |
| | | Isomer-3_ D2E1 ¹H NMR | |
| | | (300 MHz, DMSO-d6) δ | |
| | | 11.11 (s, 1H), 10.40 (s, 1H), | |
| | | 9.31 (s, 1H), 8.86 (s, 1H), 8.37 | |
| | | (s, 2H), 7.90-7.88 (m, 2H), | |
| | | 7.80-7.74 (m, 1H), 7.54-7.48 | |
| | | (m, 1H), 5.36 (d, 1H), | |
| | | 4.38-4.32 (m, 1H), 3.67 (s, | |
| | | 3H), 2.35 (s, 3H), 1.20 (d, | |
| | | 3H). | |
| | | Isomer-4_D2E2 ¹H NMR 300 | |
| | | MHz, DMSO-d₆) δ 11.11 (s, | |
| | | 1H), 10.39 (s, 1H), 9.31 (s, | |
| | | 1H), 8.86 (s, 1H), 8.37 (s, | |
| | | 2H), 7.90-7.87 (m, 2H), | |
| | | 7.80-7.74 (m, 1H), 7.53-7.48 | |
| | | (m, 1H), 5.36 (d, 1H), | |
| | | 4.40-4.30 (m, Hz, 1H), 3.67 | |
| | | (s, 3H), 2.35 (s, 3H), 1.19 (d, | |
| | | 3H). | |
| Example 48 | 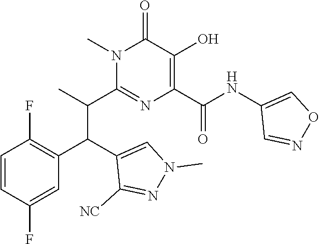 [SMILES: CC(c1nc(C(=O)Nc2cnoc2)c(O)c(=O)n1C)C(c1cc(F)ccc1F)c1cn(C)nc1C#N] | | Isomer-1_D1E1: m/z 496.2 [M + H] >98% ee Isomer-2_D1E2: m/z 496.2 [M + H] >98% ee Isomer-3_D2E1: m/z 496.2 [M + H] >96% ee Isomer-4_D2E2: m/z 496.2 [M + H] >93% ee |
| Example 50 | 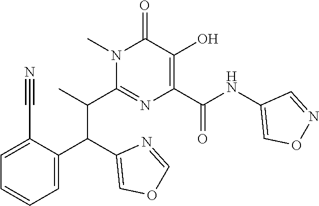 [SMILES: CC(c1nc(C(=O)Nc2cnoc2)c(O)c(=O)n1C)C(c1cocn1)c1ccccc1C#N] | Isomer-1_ D1E1: ¹H NMR (400 MHz, DMSO-d₆): δ 1.37 (d, J = 6.4 Hz, 3H). 3.55 (s, 3H), 4.10-4.15 (m, 1H), 5.11 (d, J = 10.8 Hz, 1H), 7.26 (t, J = 7.6 Hz, 1H), 7.63-7.56 (m, 2H), 7.82 (d, J = 8.0 Hz, 1H), 8.22 (s, 1H), 8.41 (s, 1H), 8.87 (s, 1H), 9.30 (s, 1H), 10.55 (bs, 1H), 11.26 (bs, 1H). Isomer-2_ D1E2: ¹H NMR (400 MHz, DMSO-d₆): δ 1.31 | Isomer-1_(D1E1)_LCMS: m/z 447.2 [M⁺ + 1]. Isomer-2_ (D1E2) LCMS m/z 448.4 [M⁺ + 1]. Isomer-3_(D2E1) LCMS: m/z 447.3 [M⁺ + 1]. Isomer-4_(D2E2) LCMS: m/z 447.3 [M⁺ + 1]. |
| | | (d, J = 6.4 Hz, 3H). 3.56 (s, | |
| | | 3H), 4.11-4.15 (m, 1H), 5.11 | |
| | | (d, J = 10.8 Hz, 1H), 7.26 (t, | |
| | | J = 7.6 Hz, 1H), 7.63-7.56 (m, | |
| | | 2H), 7.82 (d, J = 8.0 Hz, 1H), | |
| | | 8.22 (s, 1H), 8.41 (s, 1H), 8.87 | |
| | | (s, 1H), 9.30 (s, 1H), 10.54 | |
| | | (bs, 1H), 11.26 (bs, 1H). | |
| | | Isomer-3_ D2E1: ¹H NMR | |
| | | (400 MHz, DMSO-d₆): δ 1.13 | |
| | | (d, J = 6.6 Hz, 3H), 3.61 (s, | |
| | | 3H), 4.08-4.12 (m, 1H), 5.00 | |
| | | (d, J = 10.2 Hz, 1H), 7.51 (t, | |
| | | J = 7.6 Hz, 1H), 7.92-7.77 (m, | |
| | | 4H), 8.25 (s, 1H), 8.85 (s, | |
| | | 1H), 9.32 (s, 1H), 10.34 (bs, | |
| | | 1H), 11.18 (bs, 1H). | |
| | | Isomer-4_ D2E2: ¹H NMR | |
| | | (400 MHz, DMSO-d₆): δ 1.13 | |
| | | (d, J = 6.6 Hz, 3H), 3.61 (s, | |
| | | 3H), 4.08-4.12 (m, 1H), 5.00 | |
| | | (d, J = 10.2 Hz, 1H), 7.51 (t, | |
| | | J = 7.6 Hz, 1H), 7.92-7.76 (m, | |
| | | 4H), 8.25 (s, 1H), 8.85 (s, | |
| | | 1H), 9.32 (s, 1H), 10.32 (bs, | |
| | | 1H), 11.18 (bs, 1H). | |
| Example 51 | 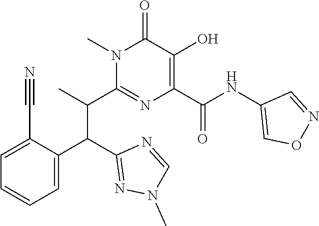 [SMILES: CC(c1nc(C(=O)Nc2cnoc2)c(O)c(=O)n1C)C(c1ncn(C)n1)c1ccccc1C#N] | Isomer-1_ D1E1: ¹H NMR (400 MHz, DMSO-d₆): δ 1.35 (d, J = 6.4 Hz, 3H), 3.53 (s, 3H), 3.86 (s, 3H), 4.16-4.20 (m, 1H), 5.22 (d, J = 10.8 Hz, 1H), 7.27 (t, J = 7.6 Hz, 1H), 7.56-7.63 (m, 2H), 7.90 (d, J = 8.0 Hz, 1H), 8.48 (s, 1H), 8.90 (s, 1H), 9.32 (s, 1H), 10.57 (s, 1H), 11.32 (s, 1H). Isomer-2_ D1E2: ¹H NMR (400 MHz, DMSO-d₆): δ 1.36 (d, J = 6.8 Hz, 3H), 3.53 (s, | Isomer-1_(D1E1)_LCMS: m/z 461.4 [M⁺ 1]. Isomer-2_ (D1E2) LCMS m/z 461.4 [M⁺ + 1]. Isomer-3_(D2E1)_LCMS: m/z 461.7 [M⁺ + 1]. Isomer-4_(D2E2) LCMS: m/z 461.4 [M⁺ + 1]. HPLC: FR-1 (Isomer-1; D1E1): R_{T} = 4.09 (99%); FR-2 (Isomer-2; D1E2): RT = 4.09 100%); FR-3 (Isomer-3; D2E1): R_{T} = 4.34 (99%); FR-4 (Isomer-4; D2E2): R_{T} = 4.35 100%). |
| | | 3H), 3.86 (s, 3H), 4.16-4.20 | |
| | | (m, 1H), 5.22 (d, J = 10.8 Hz, | |
| | | 1H), 7.27 (t, J = 7.6 Hz, 1H), | |
| | | 7.56-7.63 (m, 2H), 7.90 (d, | |
| | | J = 8.0 Hz, 1H), 8.48 (s, 1H), | |
| | | 8.90 (s, 1H), 9.32 (s, 1H), | |
| | | 10.56 (s, 1H), 11.32 (s, 1H). | |
| | | Isomer-3_ D2E1: ¹H NMR | |
| | | (400 MHz, DMSO-d₆): δ 1.08 | |
| | | (d, J = 6.4 Hz, 3H), 3.72 (s, | |
| | | 3H), 3.74 (s, 3H), 4.13-4.17 | |
| | | (m, 1H), 5.22 (d, J = 10.4 Hz, | |
| | | 1H), 7.49-7.53 (m, 1H), 7.76 | |
| | | (d, J = 3.6 Hz, 2H), 7.88 (d, | |
| | | J = 7.6 Hz, 1H), 8.30 (s, 1H), | |
| | | 8.86 (s, 1H), 9.32 (s, 1H), | |
| | | 10.34 (s, 1H), 11.07 (s, 1H). | |
| | | Isomer-4_ D2E2: ¹H NMR | |
| | | (400 MHz, DMSO-d₆): δ 1.09 | |
| | | (d, J = 6.8 Hz, 3H), 3.70 (s, | |
| | | 3H), 3.72 (s, 3H), 4.13-4.18 | |
| | | (m, 1H), 5.22 (d, J = 10.8 Hz, | |
| | | 1H), 7.49-7.53 (m, 1H), 7.76 | |
| | | (d, J = 4.0 Hz, 2H), 7.88 (d, | |
| | | J = 8.0 Hz, 1H), 8.30 (s, 1H), | |
| | | 8.86 (s, 1H), 9.33 (s, 1H), | |
| | | 10.32 (s, 1H), 11.07 (s, 1H). | |
| Example 52 | 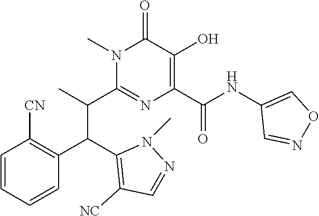 [SMILES: CC(c1nc(C(=O)Nc2cnoc2)c(O)c(=O)n1C)C(c1ccccc1C#N)c1c(C#N)cnn1C] | | Isomer-1_D1E1: m/z 485.2 [M + H] >98% ee Isomer-2_D1E2: m/z 485.2 [M + H] >97% ee Isomer-3_D2E1: m/z 485.2 [M + H] >98% ee Isomer-4_D2E2: m/z 485.2 [M + H] >98% ee |
| Example 53 | 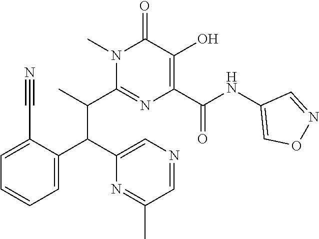 [SMILES: Cc1cncc(C(c2ccccc2C#N)C(C)c2nc(C(=O)Nc3cnoc3)c(O)c(=O)n2C)n1] | Isomer-1_ D1E1: ¹H NMR (400 MHz, DMSO-d₆): δ 1.27 (d, J = 6.0 Hz, 3H), 2.55 (s, 3H), 3.61 (s, 3H), 4.35-4.38 (m, 1H), 5.37 (d, J = 10.8 Hz, 1H), 7.26 (t, J = 7.2 Hz, 1H), 7.57-7.64 (m, 2H), 7.88 (d, J = 8.0 Hz, 1H), 8.49 (s, 1H), 8.74 (s, 1H), 8.90 (s, 1H), 9.31 (s, 1H), 10.56 (s, 1H), 11.20 (s, 1H).Isomer-2_ D1E2: ¹H NMR (400 MHz, DMSO-d₆): δ 1.26 (d, J = 6.0 Hz, 3H), 2.54 (s, 3H), 3.61 (s, 3H), 4.34-4.37 (m, 1H), 5.36 (d, | Isomer-1_(D1E1) LCMS: m/z 472.3 [M⁺ + 1]. Isomer-2_(D1E2) LCMS m/z 472.5 [M⁺ + 1]. Isomer-3_(D2E1) LCMS: m/z 472.3 [M⁺ + 1]. Isomer-4_(D2E2) LCMS: m/z 472.6 [M⁺ + 1]. HPLC: FR-1 (Isomer-1; D1E1): R_{T} = 4.63 (98%); FR-2 (Isomer-2; D1E2): R_{T} = 4.62 (97%); FR-3 (Isomer-3; D2E1): R_{T} = 4.85 100%); FR-4 (Isomer-4; D2E2): R_{T} = 4.85 (99%). |
| | | J = 10.8 Hz, 1H), 7.26 (t, | |
| | | J = 7.2 Hz, 1H), 7.56-7.64 (m, | |
| | | 2H), 7.88 (d, J = 8.0 Hz, 1H), | |
| | | 8.49 (s, 1H), 8.74 (s, 1H), 8.89 | |
| | | (s, 1H), 9.31 (s, 1H), 10.65 (s, | |
| | | 1H), 11.29 (s, 1H). | |
| | | Isomer-3_ D2E1: ¹H NMR | |
| | | (400 MHz, DMSO-d₆): δ 1.18 | |
| | | (d, J = 6.4 Hz, 3H), 2.35 (s, | |
| | | 3H), 3.72 (s, 3H), 4.27-4.31 | |
| | | (m, 1H), 5.37 (d, J = 10.4 Hz, | |
| | | 1H), 7.52 (t, J = 7.6 Hz, 1H), | |
| | | 7.78 (t, J = 7.6 Hz, 1H), 7.85 | |
| | | (d, J = 7.6 Hz, 1H), 7.90 (d, | |
| | | J = 7.6 Hz, 1H), 8.23 (s, 1H), | |
| | | 8.27 (s, 1H), 8.85 (s, 1H), 9.29 | |
| | | (s, 1H), 10.39 (s, 1H), 11.12 | |
| | | (s, 1H). | |
| | | Isomer-4_ D2E2: ¹H NMR | |
| | | (400 MHz, DMSO-d₆): δ 1.18 | |
| | | (d, J = 6.8 Hz, 3H), 2.35 (s, | |
| | | 3H), 3.72 (s, 3H), 4.27-4.32 | |
| | | (m, 1H), 5.36 (d, J = 10.8 Hz, | |
| | | 1H), 7.52 (t, J = 7.6 Hz, 1H), | |
| | | 7.78 (t, J = 8.0 Hz, 1H), 7.85 | |
| | | (d, J = 7.6 Hz, 1H), 7.90 (d, | |
| | | J = 7.6 Hz, 1H), 8.23 (s, 1H), | |
| | | 8.27 (s, 1H), 8.85 (s, 1H), 9.29 | |
| | | (s, 1H), 10.38 (s, 1H), 11.12 | |
| | | (s, 1H). | |
| Example 55 | 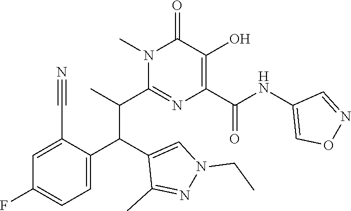 [SMILES: CCn1cc(C(c2ccc(F)cc2C#N)C(C)c2nc(C(=O)Nc3cnoc3)c(O)c(=O)n2C)c(C)n1] | Isomer-1_ D1E1: ¹H NMR (400 MHz, DMSO-d₆): δ 1.35-1.38 (m, 6H), 2.28 (s, 3H), 3.58 (s, 3H), 3.99-4.06 (m, 3H), 4.85 (d, J = 10.8 Hz, 1H), 7.47 (t, J = 8.8 Hz, 1H), 7.60 (dd, J = 8.8 Hz and 2.8 Hz, 1H), 7.79-7.82 (m, 1H), 7.99 (s, 1H), 8.86 (s, 1H), 9.32 (s, 1H), 10.48 (s, 1H), 11.27 (s, 1H).Isomer-2_ D1E2: ¹H NMR (400 MHz, DMSO-d₆): δ 1.35-1.38 (m, 6H), 2.28 (s, | Isomer-1_(D1E1) LCMS: m/z 505.5 [M⁺ + 1]. Isomer-2_ (D1E2) LCMS m/z 506.4 [M⁺ + 1]. Isomer-3_(D2E1)_LCMS: m/z 506.2 [M⁺ + 1]. Isomer- 4_(D2E2)_LCMS: m/z 506.2 [M⁺ + 1]. HPLC: FR-1 (Isomer-1; D1E1): R_{T} = 4.73 100%); FR-2 (Isomer-2; D1E2): R_{T} = 4.72 100%); FR-3 (Isomer-3; D2E1): R_{T} = 4.71 (96%); FR-4 (Isomer-4; D2E2): R_{T} = 4.70 100%). |
| | | 3H), 3.58 (s, 3H), 3.99-4.06 | |
| | | (m, 3H), 4.85 (d, J = 11.2 Hz, | |
| | | 1H), 7.47 (t, J = 8.8 Hz, 1H), | |
| | | 7.60 (dd, J = 8.8 Hz and 2.8 | |
| | | Hz, 1H), 7.79-7.82 (m, 1H), | |
| | | 7.99 (s, 1H), 8.86 (s, 1H), 9.32 | |
| | | (s, 1H), 10.46 (s, 1H), 11.27 | |
| | | (s, 1H). | |
| | | Isomer-3_ D2E1: ¹H NMR | |
| | | (400 MHz, DMSO-d₆): δ | |
| | | 1.19-1.22 (m, 6H), 1.90 (s, | |
| | | 3H), 3.53 (s, 3H), 3.90 (q, | |
| | | J = 7.2 Hz, 2H), 4.01-4.06 (m, | |
| | | 1H), 4.78 (d, J = 10.4 Hz, 1H), | |
| | | 7.70-7.73 (m, 2H), 7.85 (dd, | |
| | | J = 8.8 Hz and 2.4 Hz, 1H), | |
| | | 8.01-8.04 (m, 1H), 8.86 (s, | |
| | | 1H), 9.34 (s, 1H), 10.47 (s, | |
| | | 1H), 11.15 (s, 1H). | |
| | | Isomer-4_D2E2: ¹H NMR | |
| | | (400 MHz, DMSO-d₆): δ | |
| | | 1.19-1.22 (m, 6H), 1.90 (s, | |
| | | 3H), 3.53 (s, 3H), 3.90 (q, | |
| | | J = 6.8 Hz, 2H), 4.01-4.05 (m, | |
| | | 1H), 4.78 (d, J = 10.4 Hz, 1H), | |
| | | 7.70-7.73 (m, 2H), 7.85 (dd, | |
| | | J = 8.8 Hz and 2.8 Hz, 1H), | |
| | | 8.01-8.04 (m, 1H), 8.86 (s, | |
| | | 1H), 9.34 (s, 1H), 10.47 (s, | |
| | | 1H), 11.15 (s, 1H). | |
| Example 56 | 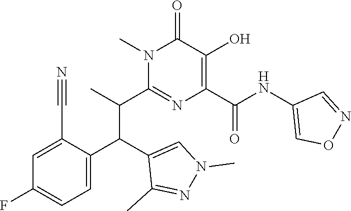 [SMILES: Cc1nn(C)cc1C(c1ccc(F)cc1C#N)C(C)c1nc(C(=O)Nc2cnoc2)c(O)c(=O)n1C] | Isomer-1_ D1E1: ¹H NMR (400 MHz, DMSO-d₆): δ 1.36 (d, J = 6.4 Hz, 3H), 2.27 (s, 3H), 3.57 (s, 3H), 3.76 (s, 3H), 3.99-4.00 (m, 1H), 4.85 (d, J = 10.8 Hz, 1H), 7.46 (t, J = 8.8 Hz, 1H), 7.60 (dd, J = 8.8 Hz and 2.8 Hz, 1H), 7.77-7.81 (m, 1H), 7.92 (s, 1H), 8.86 (s, 1H), 9.32 (s, 1H), 10.47 (s, 1H), 11.26 (s, 1H).Isomer-2_ D1E2: ¹H NMR (400 MHz, DMSO-d₆): | Isomer-1_(D1E1) LCMS: m/z 492.7 [M⁺ + 1]. Isomer-2_(D1E2) LCMS m/z 492.6 [M⁺ + 1]. Isomer-3_D2E1)_LCMS: m/z 492.2 [M⁺ + 1]. Isomer-4_(D2E2) LCMS: m/z 492.2 [M⁺ + 1]. HPLC: FR-1 (Isomer-1; D1E1): R_{T} = 4.44 (98%); FR-2 (Isomer-2; D1E2): R_{T} = 4.43 (99%); FR-3 (Isomer-3; D2E1): R_{T} = 4.67 100%); FR-4 (Isomer-4; D2E2): R_{T} = 4.67 100%). |
| | | δ 1.36 (d, J = 6.4 Hz, 3H), | |
| | | 2.27 (s, 3H), 3.57 (s, 3H), 3.75 | |
| | | (s, 3H), 3.97-4.02 (m, 1H), | |
| | | 4.85 (d, J = 11.2 Hz, 1H), 7.46 | |
| | | (t, J = 8.8 Hz, 1H), 7.60 (dd, | |
| | | J = 8.8 Hz and 2.8 Hz, 1H), | |
| | | 7.77-7.81 (m, 1H), 7.91 (s, | |
| | | 1H), 8.86 (s, 1H), 9.32 (s, | |
| | | 1H), 10.48 (s, 1H), 11.26 (s, | |
| | | 1H). | |
| | | Isomer-3_ D2E1: ¹H NMR | |
| | | (400 MHz, DMSO-d₆): δ 1.17 | |
| | | (d, J = 6.8 Hz, 3H), 1.91 (s, | |
| | | 3H), 3.55 (s, 3H), 3.62 (s, | |
| | | 3H), 4.02-4.06 (m, 1H), 4.83 | |
| | | (d, J = 10.4 Hz, 1H), | |
| | | 7.65-7.72 (m, 2H), 7.84 (dd, | |
| | | J = 8.4 Hz and 2.4 Hz, 1H), | |
| | | 7.97-8.01 (m, 1H), 8.85 (s, | |
| | | 1H), 9.34 (s, 1H), 10.43 (s, | |
| | | 1H), 11.12 (s, 1H). | |
| | | Isomer-4_ D2E2: ¹H NMR | |
| | | (400 MHz, DMSO-d₆): δ 1.17 | |
| | | (d, J = 6.4 Hz, 3H), 1.91 (s, | |
| | | 3H), 3.55 (s, 3H), 3.62 (s, | |
| | | 3H), 4.02-4.06 (m, 1H), 4.83 | |
| | | (d, J = 10.4 Hz, 1H), | |
| | | 7.66-7.72 (m, 2H), 7.84 (dd, | |
| | | J = 8.4 Hz and 2.4 Hz, 1H), | |
| | | 7.98-8.01 (m, 1H), 8.85 (s, | |
| | | 1H), 9.34 (s, 1H), 10.43 (s, | |
| | | 1H), 11.12 (s, 1H). | |
| Example 57 | 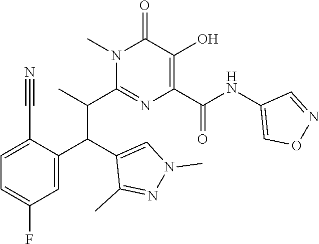 [SMILES: Cc1nn(C)cc1C(c1cc(F)ccc1C#N)C(C)c1nc(C(=O)Nc2cnoc2)c(O)c(=O)n1C] | Isomer-1_ D1E1: ¹H NMR (400 MHz, DMSO-d₆): δ 1.33 (d, J = 6.4 Hz, 3H), 2.31 (s, 3H), 3.61 (s, 3H), 3.76 (s, 3H), 3.98-4.03 (m, 1H), 4.90 (d, J = 10.8 Hz, 1H), 7.10 (t, J = 8.4 Hz, 1H), 7.70-7.75 (m, 2H), 7.94 (s, 1H), 8.85 (s, 1H), 9.31 (s, 1H), 10.43 (s, 1H), 11.24 (s, 1H). Isomer-2_ D1E2: ¹H NMR (400 MHz, DMSO-d₆): δ 1.33 (d, J = 6.4 Hz, 3H), 2.31 (s, 3H), 3.61 (s, 3H), 3.76 (s, 3H), 3.98-4.03 (m, 1H), 4.90 | Isomer-1_(D1E1) LCMS: m/z 492.5 [M⁺ + 1]. Isomer-2_(D1E2) LCMS m/z 492.5 [M⁺ + 1]. Isomer-3_(D2E1) LCMS: m/z 492.4 [M⁺ + 1]. Isomer- 4_(D2E2)_LCMS: m/z 492.4 [M⁺ + 1]. HPLC: FR-1 (Isomer-1; D1E1): R_{T} = 4.39 (99%); FR-2 (Isomer-2; D1E2): R_{T} = 4.38 100%); FR-3 (Isomer-3; D2E1): R_{T} = 4.51 (99%); FR-4 (Isomer-4; D2E2): R_{T} = 4.49 100%). |
| | | (d, J = 10.8 Hz, 1H), 7.10 (t, | |
| | | J = 8.4 Hz, 1H), 7.70-7.73 (m, | |
| | | 2H), 7.93 (s, 1H), 8.85 (s, | |
| | | 1H), 9.31 (s, 1H), 10.42 (s, | |
| | | 1H), 11.24 (s, 1H). | |
| | | Isomer-3_ D2E1: ¹H NMR | |
| | | (400 MHz, DMSO-d₆): δ 1.19 | |
| | | (d, J = 6.0 Hz, 3H), 1.91 (s, | |
| | | 3H), 3.53 (s, 3H), 3.60 (s, | |
| | | 3H), 4.03-4.08 (m, 1H), 4.82 | |
| | | (d, J = 10.4 Hz, 1H), 7.34 (t, | |
| | | J = 8.0 Hz, 1H), 7.66 (s, 1H), | |
| | | 7.91-7.96 (m, 2H), 8.84 (s, | |
| | | 1H), 9.33 (s, 1H), 10.50 (s, | |
| | | 1H), 11.11 (s, 1H). | |
| | | Isomer-4_ D2E2: ¹H NMR | |
| | | (400 MHz, DMSO-d₆): δ 1.21 | |
| | | (d, J = 6.4 Hz, 3H), 1.93 (s, | |
| | | 3H), 3.55 (s, 3H), 3.62 (s, | |
| | | 3H), 4.05-4.09 (m, 1H), 4.83 | |
| | | (d, J = 10.4 Hz, 1H), 7.36 (t, | |
| | | J = 8.4 Hz, 1H), 7.68 (s, 1H), | |
| | | 7.92-7.97 (m, 2H), 8.86 (s, | |
| | | 1H), 9.34 (s, 1H), 10.49 (s, | |
| | | 1H), 11.12 (s, 1H). | |
| Example 61 | 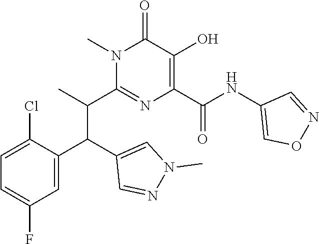 [SMILES: CC(c1nc(C(=O)Nc2cnoc2)c(O)c(=O)n1C)C(c1cnn(C)c1)c1cc(F)ccc1Cl] | Isomer-1_ D1E1: ¹H NMR (400 MHz, DMSO-d₆): δ 1.28 (d, J = 6.0 Hz, 3H), 3.65 (s, 3H), 3.80 (s, 3H), 4.04-4.07 (m, 1H), 5.11 (d, J = 10.8 Hz, 1H), 6.88 (t, J = 8.0 Hz, 1H), 7.24-7.28 (m, 1H), 7.58-7.59 (m, 2H), 7.76 (s, 1H), 8.94 (s, 1H), 9.33 (s, 1H), 10.38 (s, 1H), 11.14 (s, 1H). Isomer-2_ D1E2: ¹H NMR (400 MHz, DMSO-d₆): δ 1.28 (d, J = 6.4 Hz, 3H), 3.65 (s, 3H), 3.80 (s, 3H), 4.02-4.07 (m, 1H), 5.11 (d, J = 11.2 Hz, | Isomer-1_(D1E1) LCMS: m/z 487.3 [M⁺ + 1]. Isomer-2_ (D1E2) LCMS m/z 487.3 [M⁺ + 1]. Isomer-3_(D2E1) LCMS: m/z 487.5 [M⁺ + 1]. Isomer-4_(D2E2) LCMS: m/z 487.3 [M⁺ + 1]. HPLC: FR-1 (Isomer-1; D1E1): R_{T} = 4.55 (99%); FR-2 (Isomer-2; D1E2): R_{T} = 4.55 (99%); FR-3 (Isomer-3; D2E1): R_{T} = 4.69 (99%); FR-4 (Isomer-4; D2E2): R_{T} = 4.69 (99%). |
| | | 1H), 6.88 (t, J = 8.4 Hz, 1H), | |
| | | 7.24-7.28 (m, 1H), 7.58-7.60 | |
| | | (m, 2H), 7.76 (s, 1H), 8.94 (s, | |
| | | 1H), 9.33 (s, 1H), 10.38 (s, | |
| | | 1H), 11.14 (s, 1H). | |
| | | Isomer-3_ D2E1: ¹H NMR | |
| | | (400 MHz, DMSO-d₆): δ 1.22 | |
| | | (d, J = 6.4 Hz, 3H), 3.48 (s, | |
| | | 3H), 3.64 (s, 3H), 3.99-4.06 | |
| | | (m, 1H), 4.88 (d, J = 10.8 Hz, | |
| | | 1H), 7.04 (s, 1H), 7.17 (t, | |
| | | J = 8.8 Hz, 1H), 7.34 (s, 1H), | |
| | | 7.49-7.52 (m, 1H), 7.85 (d, | |
| | | J = 8.0 Hz, 1H), 8.95 (s, 1H), | |
| | | 9.36 (s, 1H), 10.54 (s, 1H), | |
| | | 11.38 (s, 1H).Isomer-4_D2E2: | |
| | | ¹H NMR (400 MHz, DMSO- | |
| | | d₆): δ 1.23 (d, J = 6.4 Hz, 3H), | |
| | | 3.48 (s, 3H), 3.64 (s, 3H), | |
| | | 3.99-4.06 (m, 1H), 4.88 (d, | |
| | | J = 11.2 Hz, 1H), 7.04 (s, 1H), | |
| | | 7.17 (t, J = 8.4 Hz, 1H), 7.34 | |
| | | (s, 1H), 7.49-7.52 (m, 1H), | |
| | | 7.85 (d, J = 8.0 Hz, 1H), 8.95 | |
| | | (s, 1H), 9.36 (s, 1H), 10.54 (s, | |
| | | 1H), 11.39 (s, 1H). | |
| Example 62 | 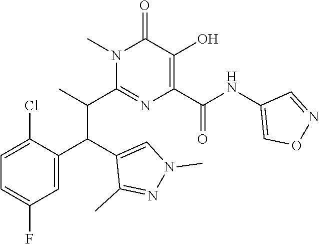 [SMILES: Cc1nn(C)cc1C(c1cc(F)ccc1Cl)C(C)c1nc(C(=O)Nc2cnoc2)c(O)c(=O)n1C] | Isomer-1_ D1E1: ¹H NMR (400 MHz, DMSO-d₆): δ 1.23 (d, J = 6.4 Hz, 3H), 1.92 (s, 3H), 3.55 (s, 3H), 3.59 (s, 3H), 3.96-4.06 (m, 1H), 4.84 (d, J = 10.0 Hz, 1H), 7.15 (t, J = 8.4 Hz, 1H), 7.47-7.50 (m, 1H), 7.59 (s, 1H), 7.83 (d, J = 10.4 Hz, 1H), 8.92 (s, 1H), 9.35 (s, 1H), 10.64 (s, 1H), 11.29 (s, 1H). Isomer-2_ D1E2: ¹H NMR (400 MHz, DMSO-d₆): δ 1.23 (d, J = 6.4 Hz, 3H), 1.92 (s, 3H), 3.55 (s, 3H), 3.59 (s, | Isomer-1_ (D1E1) LCMS: m/z 501.0 [M⁺ + 1]. Isomer-2_ (D1E2) LCMS m/z 501.2 [M⁺ + 1]. Isomer-3_(D2E1)_LCMS: m/z 501.0 [M⁺ + 1]. Isomer-4_(D2E2) LCMS: m/z 501.0 [M⁺ + 1]. HPLC: FR-1 (Isomer-1; D1E1): R_{T} = 4.67 (98%); FR-2 (Isomer-2; D1E2): R_{T} = 4.65 (99%); FR-3 (Isomer-3; D2E1): R_{T} = 4.54 (99%); FR-4 (Isomer-4; D2E2): R_{T} = 4.54 (99%). |
| | | 3H), 3.96-4.06 (m, 1H), 4.84 | |
| | | (d, J = 10.4 Hz, 1H), 7.15 (t, | |
| | | J = 8.0 Hz, 1H), 7.47-7.50 (m, | |
| | | 1H), 7.59 (s, 1H), 7.83 (d, | |
| | | J = 7.2 Hz, 1H), 8.93 (s, 1H), | |
| | | 9.35 (s, 1H), 10.63 (s, 1H), | |
| | | 11.29 (s, 1H). | |
| | | Isomer-3_ D2E1: ¹H NMR | |
| | | (400 MHz, DMSO-d₆): δ 1.30 | |
| | | (d, J = 6.4 Hz, 3H), 2.31 (s, | |
| | | 3H), 3.62 (s, 3H), 3.74 (s, | |
| | | 3H), 3.93-4.02 (m, 1H), 4.96 | |
| | | (d, J = 10.8 Hz, 1H), 6.89 (t, | |
| | | J = 8.0 Hz, 1H), 7.24-7.28 (m, | |
| | | 1H), 7.55 (dd, J = 10.0 Hz, 2.8 | |
| | | Hz, 1H), 7.81 (s, 1H), 8.95 (s, | |
| | | 1H), 9.35 (s, 1H), 10.38 (s, | |
| | | 1H), 11.17 (s, 1H). | |
| | | Isomer-4_ D2E2: ¹H NMR | |
| | | (400 MHz, DMSO-d₆): δ 1.30 | |
| | | (d, J = 6.4 Hz, 3H), 2.31 (s, | |
| | | 3H), 3.63 (s, 3H), 3.74 (s, | |
| | | 3H), 3.93-4.02 (m, 1H), 4.96 | |
| | | (d, J = 11.2 Hz, 1H), 6.89 (t, | |
| | | J = 8.0 Hz, 1H), 7.24-7.28 (m, | |
| | | 1H), 7.55 (dd, J = 10.0 Hz, 2.8 | |
| | | Hz, 1H), 7.81 (s, 1H), 8.95 (s, | |
| | | 1H), 9.35 (s, 1H), 10.38 (s, | |
| | | 1H), 11.17 (s, 1H). | |
| Example 63 | 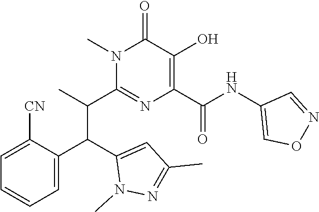 [SMILES: Cc1cc(C(c2ccccc2C#N)C(C)c2nc(C(=O)Nc3cnoc3)c(O)c(=O)n2C)n(C)n1] | Isomer-1_ D1E1: ¹H NMR (400 MHz, DMSO-d₆): δ 1.36 (d, J = 6.0 Hz, 3H), 2.12 (s, 3H), 3.54 (s, 3H), 3.93 (s, 3H), 4.05-4.15 (m, 1H), 5.14 (d, J = 10.8 Hz, 1H), 6.47 (s, 1H), 7.28 (t, J = 7.6 Hz, 1H), 7.59-7.63 (m, 2H), 7.84 (d, J = 7.6 Hz, 1H), 8.85 (s, 1H), 9.31 (s, 1H), 10.58 (s, 1H), 11.24 (s, 1H). Isomer-2_ D1E2: ¹H NMR (400 MHz, DMSO-d₆): δ 1.37 | Isomer-1_(D1E1) LCMS: m/z 474.3 [M⁺ + 1]. Isomer-2_ (D1E2) LCMS m/z 474.2 [M⁺ + 1]. Isomer-3_(D2E1) LCMS: m/z 474.3 [M⁺ + 1]. Isomer-4_(D2E2) LCMS: m/z 474.3 [M⁺ + 1]. HPLC: FR-1 (Isomer-1; D1E1): R_{T} = 4.39 (99%); FR-2 (Isomer-2; D1E2): R_{T} = 4.39 (99%); FR-3 (Isomer-3; D2E1): R_{T} = 4.46 (99%); FR-4 (Isomer-4; D2E2): RT = 4.46 (99%). |
| | | (d, J = 6.0 Hz, 3H), 2.14 (s, | |
| | | 3H), 3.56 (s, 3H), 3.94 (s, | |
| | | 3H), 4.05-4.15 (m, 1H), 5.15 | |
| | | (d, J= 10.4 Hz, 1H), 6.49 (s, | |
| | | 1H), 7.28 (t, J = 7.2 Hz, 1H), | |
| | | 7.58-7.64 (m, 2H), 7.85 (d, | |
| | | J = 7.6 Hz, 1H), 8.86 (s, 1H), | |
| | | 9.32 (s, 1H), 10.60 (s, 1H), | |
| | | 11.25 (s, 1H). | |
| | | Isomer-3_ D2E1: ¹H NMR | |
| | | (400 MHz, DMSO-d₆): δ 1.12 | |
| | | (d, J = 6.0 Hz, 3H), 1.97 (s, | |
| | | 3H), 3.64 (bs, 6H), 4.05-4.15 | |
| | | (m, 1H), 5.15 (d, J = 10.4 Hz, | |
| | | 1H), 6.21 (s, 1H), 7.51 (t, | |
| | | J = 8.0 Hz, 1H), 7.79 (t, | |
| | | J = 7.6 Hz, 1H), 7.89 (d, | |
| | | J = 7.6 Hz, 1H), 7.94 (d, | |
| | | J = 8.0 Hz, 1H), 8.82 (s, 1H), | |
| | | 9.32 (s, 1H), 10.51 (s, 1H), | |
| | | 11.13 (s, 1H). | |
| | | Isomer-4_D2E2: ¹H NMR | |
| | | (400 MHz, DMSO-d₆): δ 1.12 | |
| | | (d, J = 6.4 Hz, 3H), 1.97 (s, | |
| | | 3H), 3.64 (bs, 6H), 4.05-4.15 | |
| | | (m, 1H), 5.15 (d, J = 10.4 Hz, | |
| | | 1H), 6.21 (s, 1H), 7.51 (t, | |
| | | J = 7.6 Hz, 1H), 7.79 (t, | |
| | | J = 7.6 Hz, 1H), 7.89 (d, | |
| | | J = 8.0 Hz, 1H), 7.95 (d, | |
| | | J = 8.0 Hz, 1H), 8.82 (s, 1H), | |
| | | 9.32 (s, 1H), 10.51 (s, 1H), | |
| | | 11.14 (s, 1H). | |
| Example 64 | 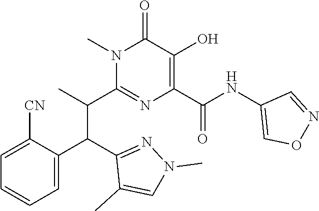 | Isomer-1_ D1E1: ¹H NMR (400 MHz, DMSO-d₆): δ 1.14-1.15 (m, 3H), 1.68 (bs, 3H), 3.64-3.68 (m, 6H), 4.16-4.18 (m, 1H), 4.95-4.98 (m, 1H), 7.22-7.23 (m, 1H), 7.45-7.49 (m, 1H), 7.75-7.86 (m, 3H), 8.83-8.85 (m, 1H), 9.32-9.33 (m, 1H), 10.37 (bs, 1H), 11.04 (bs, 1H). Isomer-2_ D1E2: ¹H NMR (400 MHz, DMSO-d₆): δ 1.15 (d, J = 6.8 Hz, 3H), 1.69 (s, | Isomer-1_(D1E1) LCMS: m/z 474.4 [M⁺ + 1]. Isomer-2_ (D1E2) LCMS m/z 474.2 [M⁺ + 1]. Isomer-3_(D2E1) LCMS: m/z 474.2 [M⁺ + 1]. Isomer-4_(D2E2) LCMS: m/z 474.4 [M⁺ + 1]. HPLC: FR-1 (Isomer-1; D1E1): R_{T} = 5.13 100%); FR-2 (Isomer-2; D1E2): R_{T} = 5.13 100%); FR-3 (Isomer-3; D2E1): R_{T} = 4.69 (99%); FR-4 (Isomer-4; D2E2): R_{T} = 4.70 (99%). |
| | | 3H), 3.65 (s, 3H), 3.67 (s, | |
| | | 3H), 4.14-4.19 (m, 1H), 4.97 | |
| | | (d, J = 10.4 Hz, 1H), 7.22 (s, | |
| | | 1H), 7.48 (t, J = 7.6 Hz, 1H), | |
| | | 7.74-7.81 (m, 2H), 7.86 (d, | |
| | | J = 7.6 Hz, 1H), 8.84 (s, 1H), | |
| | | 9.33 (s, 1H), 10.39 (s, 1H), | |
| | | 11.05 (s, 1H). | |
| | | Isomer-3_ D2E1: ¹H NMR | |
| | | (400 MHz, DMSO-d₆): δ 1.38 | |
| | | (d, J = 6.4 Hz, 3H), 2.11 (s, | |
| | | 3H), 3.52 (s, 3H), 3.80 (s, | |
| | | 3H), 4.17-4.21 (m, 1H), 4.99 | |
| | | (d, J = 10.8 Hz, 1H), 7.23 (t, | |
| | | J = 7.6 Hz, 1H), 7.37 (s, 1H), | |
| | | 7.53-7.56 (m, 2H), 7.76 (d, | |
| | | J = 8.0 Hz, 1H), 8.85 (s, 1H), | |
| | | 9.23 (s, 1H), 10.15 (s, 1H), | |
| | | 11.13 (s, 1H). | |
| | | Isomer-4_ D2E2: ¹H NMR | |
| | | (400 MHz, DMSO-d₆): δ 1.38 | |
| | | (d, J = 5.6 Hz, 3H), 2.09 (s, | |
| | | 3H), 3.51 (s, 3H), 3.79 (s, | |
| | | 3H), 4.13-4.17 (m, 1H), 4.99 | |
| | | (d, J = 10.4 Hz, 1H), 7.21 (t, | |
| | | J = 7.2 Hz, 1H), 7.41 (s, 1H), | |
| | | 7.52-7.58 (m, 2H), 7.73 (d, | |
| | | J = 8.0 Hz, 1H), 8.86 (s, 1H), | |
| | | 9.31 (s, 1H), 10.52 (s, 1H), | |
| | | 11.31 (s, 1H). | |
| Example 65 | 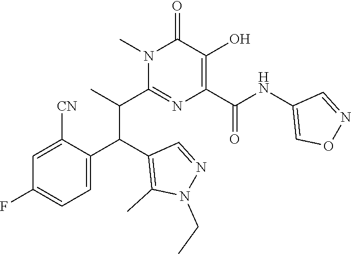 [SMILES: CCn1ncc(C(c2ccc(F)cc2C#N)C(C)c2nc(C(=O)Nc3cnoc3)c(O)c(=O)n2C)c1C] | Isomer-1_ D1E1: ¹H NMR (400 MHz, DMSO-d₆): δ 1.26-1.30 (m, 6H), 2.43 (s, 3H), 3.60 (s, 3H), 4.02-4.06 (m, 3H), 4.89 (d, J = 10.8 Hz, 1H), 7.45 (t, J = 8.4 Hz, 1H), 7.59 (t, J = 4.2 Hz, 1H), 7.73 (s, 1H), 7.90 (q, J = 6.8 Hz, 1H), 8.86 (s, 1H), 9.32 (s, 1H), 10.48 (bs, 1H), 11.22 (s, 1H). Isomer-2_ D1E2: ¹H NMR (400 MHz, DMSO-d₆): δ 1.26-1.30 (m, 6H), 2.44 (s, 3H), 3.60 (s, 3H), 4.02-4.07 (m, 3H), 4.89 (d, J = 9.6 Hz, | Isomer-1_ (D1E1) LCMS: m/z 506.4 [M⁺ + 1]. Isomer-2_ (D1E2) LCMS m/z 506.4 [M⁺ + 1]. Isomer-3_(D2E1) LCMS: m/z 506.6 [M⁺ + 1]. Isomer-4_(D2E2) LCMS: m/z 506.6 [M⁺ + 1]. HPLC: FR-1 (Isomer-1; D1E1): R_{T} = 4.77 (95 %); FR-2 (Isomer-2; D1E2): R_{T} = 4.77 100 %); FR-3 (Isomer-3; D2E1): R_{T} = 4.74 (98 %); FR-4 (Isomer-4; D2E2): RT = 4.73 (98 %). |
| | | 1H), 7.46 (t, J = 8.4 Hz, 1H), | |
| | | 7.59 (d, J = 6.4 Hz, 1H), 7.73 | |
| | | (s, 1H), 7.90 (s, 1H), 8.86 (s, | |
| | | 1H), 9.32 (s, 1H), 10.48 (bs, | |
| | | 1H), 11.22 (s, 1H). | |
| | | Isomer-3_ D2E1: ¹H NMR | |
| | | (400 MHz, DMSO-d₆): δ 1.02 | |
| | | (t, J = 7.2 Hz, 3H), 1.21 (d, | |
| | | J = 6 Hz, 3H), 2.02 (s, 3H), | |
| | | 3.47 (s, 3H), 3.84 (q, J = 6.8 | |
| | | Hz, 3H), 4.08-4.12 (m, 1H), | |
| | | 4.72 (d, J = 10.4 Hz, 1H), 7.40 | |
| | | (s, 1H), 7.70 (t, J = 7.2 Hz, | |
| | | 1H), 7.83 (d, J = 6.0 Hz, 1H), | |
| | | 8.17 (t, J = 6.8 Hz, 1H), 8.87 | |
| | | (s, 1H), 9.33 (s, 1H), 10.89 | |
| | | (bs, 1H), 11.21 (bs, 1H). | |
| | | Isomer-4_D2E2: ¹H NMR | |
| | | (400 MHz, DMSO-d₆): δ 1.02 | |
| | | (t, J = 7.0 Hz, 3H), 1.22 (d, | |
| | | J = 6.0 Hz, 3H), 2.01 (s, 3H), | |
| | | 3.47 (s, 3H), 3.84 (q, J = 6.8 | |
| | | Hz, 2H), 4.08-4.13 (m, 1H), | |
| | | 4.72 (d, J = 6.8 Hz, 1H), 7.40 | |
| | | (s, 1H), 7.71 (t, J = 8.8 Hz, | |
| | | 1H), 7.88 (dd, J = 7.2 Hz and | |
| | | J = 2.4 Hz, 1H), 8.16 (t, | |
| | | J = 6.8 Hz, 1H), 8.88 (s, 1H), | |
| | | 9.34 (s, 1H), 10.77 (bs, 1H), | |
| | | 11.18 (s, 1H). | |
| Example 66 | 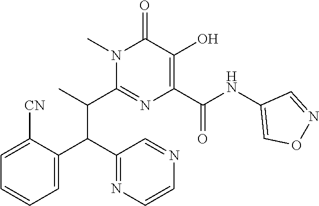 [SMILES: CC(c1nc(C(=O)Nc2cnoc2)c(O)c(=O)n1C)C(c1cnccn1)c1ccccc1C#N] | Isomer-1_ D1E1: ¹H NMR (400 MHz, DMSO-d₆): δ 1.26 (d, J = 6.4 Hz, 3H), 3.62 (s, 3H), 4.37-4.42 (m, 1H), 5.44 (d, J = 11.2 Hz, 1H), 7.27 (t, J = 7.6 Hz, 1H), 7.58 (t, J = 7.6 Hz, 1H), 7.65 (d, J = 7.6 Hz, 1H), 7.89 (d, J = 8 Hz, 1H), 8.62 (s,1H), 8.73 (s,1H), 8.90 (s, 1H), 8.94 (s, 1H), 9.33 (s, 1H), 10.57 (s, 1H), 11.19 (s, 1H) Isomer-2_ D1E2: ¹H NMR | Isomer-1_(D1E1) LCMS: m/z 458.2 [M⁺ + 1]. Isomer-2_(D1E2) LCMS m/z 458.2 [M⁺ + 1]. Isomer-3_(D2E1) LCMS: m/z 458.3 [M⁺ + 1]. Isomer-4_(D2E2) LCMS: m/z 458.3 [M⁺ + 1]. HPLC: FR-1 (Isomer-1; D1E1): R_{T} = 4.43 (98%); FR-2 (Isomer-2; D1E2): R_{T} = 4.43 100%); FR-3 (Isomer-3; D2E1): R_{T} = 4.61 (99%); FR-4 (Isomer-4; D2E2): RT = 4.61 (98%). |
| | | (400 MHz, DMSO-d₆): ): δ | |
| | | 1.27 (d, J = 6.4 Hz, 3H), 3.63 | |
| | | (s, 3H), 4.38-4.43 (m, 1H), | |
| | | 5.45 (d, J = 10.8 Hz, 1H), 7.28 | |
| | | (t, J = 7.6 Hz, 1H), 7.44 (t, | |
| | | J = 7.6 Hz, 1H), 7.66 (d, | |
| | | J = 7.6 Hz, 2H), 7.63 (d, J = 8 | |
| | | Hz, 2H), 8.74 (s, 1H), 8.93 (s, | |
| | | 1H), 9.33 (s, 1H), 11.20 (s, | |
| | | 1H). | |
| | | Isomer-3_ D2E1: ¹H NMR | |
| | | (400 MHz, DMSO-d₆ δ 1.21 | |
| | | (d, J = 6.4 Hz, 3H), 3.17 (s, | |
| | | 3H), 3.67 (s, 3H), 4.32-4.36 | |
| | | (m, 1H), 5.40 (d, J = 10.4 Hz, | |
| | | 1H), 7.522 (t, J = 7.6 Hz, 1H), | |
| | | 7.78 (t, J = 7.6 Hz, 1H), 7.90 | |
| | | (d, J = 7.6 Hz, 2H), 8.37 (s, | |
| | | 1H), 8.48 (s, 1H), 8.54 (s, | |
| | | 1H), 8.86 (s, 1H), 9.31 (s, | |
| | | 1H), 10.43 (s, 1H), 11.15 (s, | |
| | | 1H). | |
| | | Isomer-4_ D2E2: ¹H NMR | |
| | | (400 MHz, DMSO-d δ 1.21 | |
| | | (d, J = 6.4 Hz, 3H), 3.67 (s, | |
| | | 3H), 4.32-4.44 (s, 1H), 5.40 | |
| | | (d, J = 10.4 Hz, 1H), 7.52 (t, | |
| | | J = 7.6 Hz, 1H), 7.78 (t, | |
| | | J = 7.6 Hz, 1H), 7.91 (d, | |
| | | J = 7.6 Hz, 2H), 8.37 (s,1H), | |
| | | 8.49 (s, 1H), 8.54 (s, 1H), 8.85 | |
| | | (s, 1H), 8.86 (s, 1H), 9.31 (s, | |
| | | 1H). | |
| Example 67 | 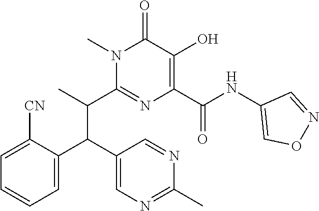 [SMILES: Cc1ncc(C(c2ccccc2C#N)C(C)c2nc(C(=O)Nc3cnoc3)c(O)c(=O)n2C)cn1] | Isomer-1_ D1E1: ¹H NMR (400 MHz, DMSO-d₆): δ 1.24 (d, J = 5.2 Hz, 3H), 2.61 (s, 3H), 3.67 (s, 3H), 4.39-4.45 (m, 1H), 5.25 (d, J = 11.2 Hz, 1H), 7.27 (t, J = 7.2 Hz, 1H), 7.60-7.66 (m, 2H), 7.95 (d, J = 8.0 Hz, 1H), 8.87 (s, 1H), 8.99 (s, 2H), 9.30 (s, 1H), 10.56 (bs, 1H), 11.11 (s, 1H). Isomer-2_ D1E2: ¹H NMR (400 MHz, DMSO-d₆): δ 1.24 | Isomer-1_(D1E1) LCMS: m/z 472.2 [M⁺ + 1]. Isomer-2_(D1E2) LCMS m/z 472.2 [M⁺ + 1]. Isomer-3_(D2E1) LCMS: m/z 471.1 [M⁺ + 1]. Isomer-4_(D2E2) LCMS: m/z 471.9 [M⁺ + 1]. HPLC: FR-1 (Isomer-1; D1E1): R_{T} = 4.25 (97%); FR-2 (Isomer-2; D1E2): R_{T} = 4.27 (96%); FR- 3(Isomer-3; D2E1): R_{T} = 4.26 100%); FR- 4 (Isomer-4; D2E2): R_{T} = 4.30 (91%). |
| | | (d, J = 5.2 Hz, 3H), 2.62 (s, | |
| | | 3H), 3.67 (s, 3H), 4.39-4.45 | |
| | | (m, 1H), 5.25 (d, J = 10.8 Hz, | |
| | | 1H), 7.28 (t, J = 7.2 Hz, 1H), | |
| | | 7.61-7.67 (m, 2H), 7.95 (d, | |
| | | J = 7.6 Hz, 1H), 8.88 (s, 1H), | |
| | | 9.00 (s, 2H), 9.31 (s, 1H), | |
| | | 10.62 (bs, 1H), 11.12 (s, 1H). | |
| | | Isomer-3_ D2E1: ¹H NMR | |
| | | (400 MHz, DMSO-d₆): δ 1.28 | |
| | | (d, J = 6.4 Hz, 3H), 2.44 (s, | |
| | | 3H), 3.58 (s, 3H), 4.32-4.37 | |
| | | (m, 1H), 5.09 (d, J = 11.2 Hz, | |
| | | 1H), 7.51 (t, J = 7.6 Hz, 1H), | |
| | | 7.83-7.88 (m, 2H), 8.21 (d, | |
| | | J = 7.6 Hz, 1H), 8.65 (s, 2H), | |
| | | 8.87 (s, 1H), 9.34 (s, 1H), | |
| | | 10.69 (bs, 1H), 11.18 (s, 1H). | |
| | | Isomer-4_ D2E2: ¹H NMR | |
| | | (400 MHz, DMSO-d₆): δ 1.29 | |
| | | (d, J = 6.0 Hz, 3H), 2.45 (s, | |
| | | 3H), 3.58 (s, 3H), 4.32-4.37 | |
| | | (m, 1H), 5.09 (d, J = 11.2 Hz, | |
| | | 1H), 7.52 (t, J = 7.2 Hz, 1H), | |
| | | 7.83-7.89 (m, 2H), 8.22 (d, | |
| | | J = 7.6 Hz, 1H), 8.65 (s, 2H), | |
| | | 8.88 (s, 1H), 9.35 (s, 1H), | |
| | | 10.76 (s, 1H), 11.20 (s, 1H). | |
| Example 79 | 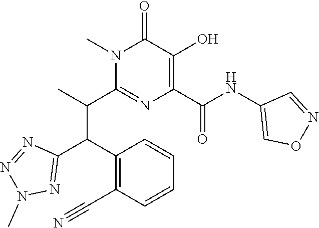 [SMILES: CC(c1nc(C(=O)Nc2cnoc2)c(O)c(=O)n1C)C(c1nnn(C)n1)c1ccccc1C#N] | Isomer-1_ D1E1: ¹H NMR (400 MHz, DMSO-d₆): δ 1.37 (d, J = 6.4 Hz, 3H), 3.57 (s, 3H), 4.27-4.33 (m, 1H), 4.39 (s, 3H), 5.53 (d, J = 10.8 Hz, 1H), 7.32 (t, J = 7.6 Hz, 1H), 7.61-7.68 (m, 2H), 7.91 (d, J = 8.0 Hz, 1H), 8.90 (s, 1H), 9.32 (s, 1H), 10.64 (bs, 1H), 11.32 (s, 1H). Isomer-2_ D1E2: ¹H NMR (400 MHz, DMSO-d₆): δ 1.36 (d, J = 6.0 Hz, 3H), 3.56 (s, 3H), 4.25-4.30 (m, 1H), 4.38 | Isomer-1 (D1E1)_LCMS: m/z 462.5 [M⁺ + 1]. Isomer-2_(D1E2) LCMS: m/z 462.5 [M⁺ + 1]. Isomer-3_(D2E1) LCMS: m/z 462.2 [M⁺ + 1]. Isomer-4_(D2E2) LCMS: m/z 462.2 [M⁺ + 1]. |
| | | (s, 3H), 5.52 (d, J = 10.8 Hz, | |
| | | 1H), 7.31 (t, J = 7.2 Hz, 1H), | |
| | | 7.59-7.67 (m, 2H), 7.91 (d, | |
| | | J = 8.0 Hz, 1H), 8.88 (s, 1H), | |
| | | 9.31 (s, 1H), 10.64 (bs, 1H), | |
| | | 11.30 (s, 1H). | |
| | | Isomer-3_ D2E1: ¹H NMR | |
| | | (400 MHz, DMSO-d₆): δ 1.12 | |
| | | (d, J = 6.8 Hz, 3H), 3.74 (s, | |
| | | 3H), 4.23 (s, 3H), 4.26-4.30 | |
| | | (m, 1H), 5.50 (d, J = 10.8 Hz, | |
| | | 1H), 7.55 (t, J = 6.8 Hz, 1H), | |
| | | 7.76-7.83 (m, 2H), 7.92 (d, | |
| | | J = 7.6 Hz, 1H), 8.85 (s, 1H), | |
| | | 9.31 (s, 1H), 10.32 (bs, 1H), | |
| | | 11.19 (s, 1H). | |
| | | Isomer-4_ D2E2: ¹H NMR | |
| | | (400 MHz, DMSO-d₆): δ 1.12 | |
| | | (d, J = 6.4 Hz, 3H), 3.74 (s, | |
| | | 3H), 4.23 (s, 3H), 4.27-4.29 | |
| | | (m, 1H), 5.50 (d, J = 10.8 Hz, | |
| | | 1H), 7.55 (t, J = 7.2 Hz, 1H), | |
| | | 7.76-7.83 (m, 2H), 7.92 (d, | |
| | | J = 6.8 Hz, 1H), 8.85 (s, 1H), | |
| | | 9.31 (s, 1H), 10.33 (bs, 1H), | |
| | | 11.19 (s, 1H). | |
| Example 80 | 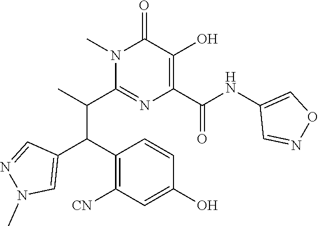 | Isomer-1_ D1E1: ¹H NMR (400 MHz, DMSO-d₆): δ 1.31 (d, J = 3.9 Hz, 3H), 3.57 (s, 3H), 3.81 (s, 3H), 3.82-3.99 (m, 1H), 4.83 (d, J = 10.8 Hz, 1H), 6.85 (s, 1H), 6.95 (d, J = 8.8 Hz, 1H), 7.54-7.58 (m, 2H), 7.74 (s, 1H), 8.86 (s, 1H), 9.30 (s, 1H), 9.91 (s, 1H), 10.52 (s, 1H), 11.28 (s, 1H).Isomer-2_ D1E2: ¹H NMR (400 MHz, DMSO-d₆): δ 1.32 (d, J = 6.0 Hz, 3H), | Isomer-1_(D1E1) LCMS: m/z 476.4 [M⁺ + 1]. Isomer-2_(D1E2) _LCMS: m/z 476.4 [M⁺ + 1]. Isomer-3_(D2E1)_LCMS: m/z 476.2 [M⁺ + 1]. Isomer- 4_(D2E2)_LCMS: m/z 476.2 [M⁺ + 1]. HPLC: FR-1 (Isomer-1; D1E1): R_{T} = 3.95 (96%); FR-2 (Isomer-2; D1E2): R_{T} = 3.95 (99%); FR-3 (Isomer-3; D2E1): R_{T} = 4.01 100%); FR-4 (Isomer-4; D2E2): R_{T} = 4.01 100%). |
| | | 3.57 (s, 3H), 3.81 (s, 3H), | |
| | | 3.82-3.99 (m, 1H), 4.84 (d, | |
| | | J = 10.8 Hz, 1H), 6.85 (s, 1H), | |
| | | 6.95 (d, J = 8.0 Hz, 1H), 7.56- | |
| | | 7.60 (m, 2H), 7.74 (s, 1H), | |
| | | 8.88 (s, 1H), 9.31 (s, 1H), 9.92 | |
| | | (s, 1H), 10.58 (s, 1H), 11.28 | |
| | | (s, 1H). | |
| | | Isomer-3_D2E1: ¹H NMR | |
| | | (400 MHz, DMSO-d₆): δ 1.12 | |
| | | (bs, 3H), 2.99 (s, 3H), 3.65 (s, | |
| | | 3H), 3.82-3.99 (m, 1H), 4.74 | |
| | | (d, J = 10.8 Hz, 1H), 7.05 (s, | |
| | | 1H), 7.10 (bs, 1H), 7.15-7.20 | |
| | | (m, 1H), 7.32 (s, 1H), | |
| | | 7.60-7.72 (m, 1H), 8.85 (s, | |
| | | 1H), 9.31 (s, 1H), 10.58 (s, | |
| | | 1H), 11.28 (s, 1H).Isomer- | |
| | | 4_D2E2: ¹H NMR (400 MHz, | |
| | | DMSO-d₆): δ 1.13 (bs, 3H), | |
| | | 2.96 (s, 3H), 3.65 (s, 3H), | |
| | | 3.82-3.99 (m, 1H), 4.72 (d, | |
| | | J = 10.8 Hz, 1H), 7.06 (s, 1H), | |
| | | 7.10 (bs, 1H), 7.15-7.20 (m, | |
| | | 1H), 7.33 (s, 1H), 7.65-7.72 | |
| | | (m, 1H), 8.86 (s, 1H), 9.32 (s, | |
| | | 1H), 10.14 (s, 1H), 11.22 (s, | |
| | | 1H). | |
| Example 81 | 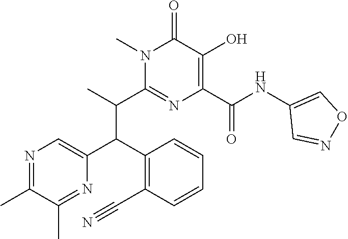 [SMILES: Cc1ncc(C(c2ccccc2C#N)C(C)c2nc(C(=O)Nc3cnoc3)c(O)c(=O)n2C)nc1C] | Isomer-1_ D1E1: ¹H NMR (400 MHz, DMSO-d₆): δ 1.28 (d, J = 6 Hz, 3H), 2.53 (s, 6H), 3.64 (s, 3H), 4.34-4.39 (m, 1H), 5.32 (d, J = 10.8 Hz, 1H), 7.27 (t, J = 7.6 Hz, 1H), 7.58-7.65 (m, 2H), 7.89 (d, J = 8 Hz, 1H), 8.61 (s, 1H), 8.92 (s, 1H), 9.34 (s, 1H), 10.60 (bs, 1H), 11.24 (s, 1H). Isomer-2_ D1E2: ¹H NMR (400 MHz, DMSO-d₆): δ 1.28 (d, J = 7.6 Hz, 3H), 2.51 (s, 6H), 3.62 (s, 3H), 4.33-4.37 | Isomer-1_(D1E1) LCMS: m/z 486.2 [M⁺ + 1]. Isomer-2_(D1E2)_LCMS: m/z 486.2 [M⁺ + 1]. Isomer-3_(D2E1)_LCMS: m/z 486.2 [M⁺ + 1]. Isomer- 4_(D2E2)_LCMS: m/z 486.2 [M⁺ + 1]. HPLC: FR-1 (Isomer-1; D1E1): R_{T} = 4.81 100%); FR-2 (Isomer-2; D1E2): R_{T} = 4.80 100%); FR-3 (Isomer-3; D2E1): R_{T} = 4.95 (99%); FR-4 (Isomer-4; D2E2): R_{T} = 4.95 (97%). |
| | | (m, 1H), 5.32 (d, J = 10.8 Hz, | |
| | | 1H), 7.26 (t, J = 7.4 Hz, 1H), | |
| | | 7.56-7.64 (m, 2H), 7.88 (d, | |
| | | J = 8.0 Hz, 1H), 8.60 (s, 1H), | |
| | | 8.91 (s, 1H), 9.32 (s, 1H), | |
| | | 10.56 (bs, 1H), 11.22 (s, 1H). | |
| | | Isomer-3_ D2E1: ¹H NMR | |
| | | (400 MHz, DMSO-d₆): δ 1.16 | |
| | | (d, J = 6 Hz, 3H), 2.33 (s, 6H), | |
| | | 3.72 (s, 3H), 4.28-4.32 (m, | |
| | | 1H), 5.32 (d, J = 10.8 Hz, 1H), | |
| | | 7.51 (t, J = 7.2 Hz, 1H), | |
| | | 7.75-7.90 (m, 3H), 8.08 (s, | |
| | | 1H), 8.85 (s, 1H), 9.30 (s, | |
| | | 1H), 10.43 (bs, 1H), 11.10 (s, | |
| | | 1H). | |
| | | Isomer-4_ D2E2: ¹H NMR | |
| | | (400 MHz, DMSO-d₆): δ 1.16 | |
| | | (d, J = 6.8 Hz, 3H), 2.35 (s, | |
| | | 6H), 3.72 (s, 3H), 4.29-4.33 | |
| | | (m, 1H), 5.32 (d, J = 10.8 Hz, | |
| | | 1H), 7.53 (t, J = 7.2 Hz 1H), | |
| | | 7.76-7.91 (m, 3H), 8.10 (s, | |
| | | 1H), 8.86 (s, 1H), 9.30 (s, | |
| | | 1H), 10.46 (s, 1H), 11.12 (s, | |
| | | 1H). | |
| Example 82 | 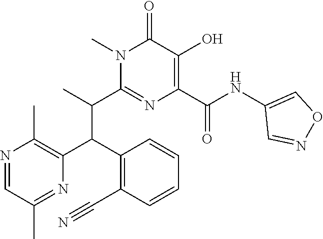 [SMILES: Cc1cnc(C)c(C(c2ccccc2C#N)C(C)c2nc(C(=O)Nc3cnoc3)c(O)c(=O)n2C)n1] | Isomer-1_ D1E1: ¹H NMR (400 MHz, DMSO-d₆): ): δ 1.31 (d, J = 6.4 Hz, 3H), 2.58 (s, 3H), 2.74 (s, 3H), 3.49 (s, 3H), 4.37-4.44 (m, 1H), 5.31 (d, J = 12.4 Hz, 1H), 7.29 (t, J = 7.6 Hz, 1H), 7.57-7.64 (m, 2H), 7.84 (d, J = 8.0 Hz, 1H), 8.37 (s, 1H), 8.88 (s, 1H), 9.33 (s, 1H), 10.57 (s, 1H), 11.34 (s, 1H).Isomer-1_ D1E2: ¹H NMR (400 MHz, DMSO-d₆): ): δ 1.31 (d, J = 6.4 Hz, 3H), 2.58 (s, 3H), 2.74 (s, 3H), 3.49 (s, 3H), 4.36-4.40 (m, 1H), | Isomer-1_ (D1E1) m/z 486.2 LCMS: [M⁺ + 1]. Isomer-2_ (D1E2) LCMS: m/z 486.4 [M⁺ + 1]. Isomer-3_(D2E1)_LCMS: m/z 486.4 [M⁺ + 1]. Isomer- 4_(D2E2)_LCMS: m/z 486.2 [M⁺ + 1]. HPLC: FR-1 (Isomer-1; D1E1): R_{T} = 2.76 (98%); FR-2 (Isomer-2; D1E2): R_{T} = 2.75 (99%); FR-3 (Isomer-3; D2E1): R_{T} = 2.90 (98%); FR-4 (Isomer-4; D2E2): R_{T} = 4.99 (99%). |
| | | 5.31(d, J = 12.4 Hz, 1H), 7.29 | |
| | | (t, J = 7.6 Hz, 1H), 7.57-7.64 | |
| | | (m, 2H), 7.84 (d, J = 7.6 Hz, | |
| | | 1H), 8.37 (s, 1H), 8.88 (s, | |
| | | 1H), 9.33 (s, 1H), 10.57 (s, | |
| | | 1H), 11.35 (s, 1H). | |
| | | Isomer-1_ D2E1: ¹H NMR | |
| | | (400 MHz, DMSO-d₆): ): δ | |
| | | 1.18 (d, J = 8.0 Hz, 3H), 2.34 | |
| | | (s, 3H), 2.39 (s, 3H), 3.76 (s, | |
| | | 3H), 4.17-4.22 (m, 1H), 5.35 | |
| | | (d, J = 10.0 Hz, 1H), 7.52 (t, | |
| | | J = 7.2 Hz, 1H), 7.66-7.75 (m, | |
| | | 2H), 7.92 (d, J = 8.0 Hz, 1H), | |
| | | 8.14 (s, 1H), 8.79 (s, 1H), 9.27 | |
| | | (s, 1H), 10.41 (s, 1H), 11.05 | |
| | | (s, 1H). | |
| | | Isomer-1_ D2E2: ¹H NMR | |
| | | (400 MHz, DMSO-d₆): ): δ | |
| | | 1.21 (d, J = 6.8 Hz, 3H), 2.36 | |
| | | (s, 3H), 2.40 (s, 3H), 3.78 (s, | |
| | | 3H), 4.20-4.24 (m, 1H), 5.37 | |
| | | (d, J = 10.4 Hz, 1H), 7.52 (t, | |
| | | J = 7.2 Hz, 1H), 7.67-7.76 (m, | |
| | | 2H), 7.93 (d, J = 7.6 Hz, 1H), | |
| | | 8.15 (s, 1H), 8.81 (s, 1H), 9.28 | |
| | | (s, 1H), 10.39 (s, 1H), 11.04 | |
| | | (s, 1H). | |
| Example 83 | 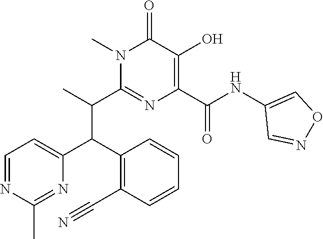 [SMILES: Cc1nccc(C(c2ccccc2C#N)C(C)c2nc(C(=O)Nc3cnoc3)c(O)c(=O)n2C)n1] | Isomer-1_ D1E1: ¹H NMR (400 MHz, DMSO-d₆): δ 1.31 (d, J = 8 Hz, 3H), 2.68 (s, 3H), 3.63 (s, 3H), 4.36-4.41 (m, 1H), 5.26 (d, J = 12.0 Hz, 1H), 7.27 (t, J = 8.0 Hz, 1H), 7.58-7.67 (m, 3H), 7.88 (d, J = 8.0 Hz, 1H), 8.74 (d, J = 8 Hz, 1H), 8.90 (s, 1H), 9.32 (s, 1H), 10.56 (bs, 1H), 11.23 (bs, 1H).Isomer-2_ D1E2: ¹H NMR (400 MHz, DMSO-d₆): δ 1.31 (d, J = 6.4 Hz, 3H), 2.68 (s, 3H), 3.62 (s, 3H), 4.36-4.40 (m, 1H), 5.26 (d, | Isomer-1 (D1E1) LCMS: m/z 473.5 [M⁺ + 1]. Isomer-2_ (D1E2) LCMS: m/z 473.4 [M⁺ 1]. Isomer-3_(D2E1)_LCMS: m/z 472.0 [M⁺ + 1]. Isomer- 4_(D2E2)_LCMS: m/z 473.0 [M⁺ + 1]. HPLC: FR-1 (Isomer-1; D1E1): R_{T} = 4.47 (95%); FR-2 (Isomer-2; D1E2): R_{T} = 4.47 (98%); FR-3 (Isomer-3; D2E1): R_{T} = 4.63 (99%); FR-4 (Isomer-4; D2E2): R_{T} = 4.61 (97%). |
| | | J = 10.8 Hz, 1H), 7.27 (t, | |
| | | J = 8.8 Hz, 1H), 7.58-7.66 (m, | |
| | | 3H), 7.87 (d, J = 4.0 Hz, 1H), | |
| | | 8.74 (d, J = 5.2 Hz, 1H), 8.90 | |
| | | (s, 1H), 9.32 (s, 1H), 10.57 | |
| | | (bs, 1H), 11.23 (bs, 1H). | |
| | | Isomer-3_ D2E1: ¹H NMR | |
| | | (400 MHz, DMSO-d₆): δ 1.14 | |
| | | (d, J = 6.0 Hz, 3H), 2.46 (s, | |
| | | 3H), 3.79 (s, 3H), 4.26-4.30 | |
| | | (m, 1H), 5.31 (d, J = 10.4 Hz, | |
| | | 1H), 7.05 (d, J = 4.8 Hz, 1H), | |
| | | 7.54 (t, J = 7.2 Hz, 1H), 7.80 | |
| | | (d, J = 6.4 Hz, 2H), 7.93 (d, | |
| | | J = 7.2 Hz, 1H), 8.46 (d, | |
| | | J = 5.2 Hz, 1H), 8.86 (s, 1H), | |
| | | 9.30 (s, 1H), 10.33 (bs, 1H), | |
| | | 11.12 (bs, 1H). | |
| | | Isomer-4_ D2E2: ¹H NMR | |
| | | (400 MHz, DMSO-d₆): δ 1.11 | |
| | | (d, J = 6.8 Hz, 3H), 2.45 (s, | |
| | | 3H), 3.77 (s, 3H), 4.24-4.28 | |
| | | (m, 1H), 5.29 (d, J = 10.4 Hz, | |
| | | 1H), 7.04 (d, J = 4.8 Hz, 1H), | |
| | | 7.52 (t, J = 6.8 Hz, 1H), 7.76 | |
| | | (d, J = 8 Hz, 2H), 7.91 (d, | |
| | | J = 7.6 Hz, 1H), 8.44 (d, | |
| | | J = 5.2 Hz, 1H), 8.84 (s, 1H), | |
| | | 9.28 (s, 1H) 10.35 (bs, 1H), | |
| | | 11.11 (bs, 1H). | |
| Example 84 | 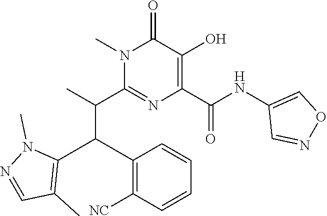 [SMILES: Cc1cnn(C)c1C(c1ccccc1C#N)C(C)c1nc(C(=O)Nc2cnoc2)c(O)c(=O)n1C] | Isomer-1_ D1E1: ¹H NMR (400 MHz, DMSO-d₆): δ 1.33 (d, J = 3.2 Hz, 3H), 2.14 (s, 3H), 3.60 (s, 3H), 4.06 (s, 3H), 4.08-4.12 (m, 1H), 5.31 (d, J = 10 Hz, 1H), 7.23 (s, 1H), 7.32 (m, 1H), 7.61 (s, 2H), 7.68 (d, J = 7.6 Hz, 1H), 8.83 (s, 1H), 9.30 (s, 1H), 10.57 (bs, 1H), 11.18 (s, 1H). Isomer-2_ D1E2: ¹H NMR (400 MHz, DMSO-d₆): δ 1.33 | Isomer-1 (D1E1) LCMS: m/z 474.4 [M⁺ + 1]. Isomer-2_(D1E2) LCMS: m/z 474.4 [M⁺ + 1]. HPLC: FR-1 (Isomer-1; D1E1): R_{T} = 4.43 100%); FR-2 (Isomer-2; D1E2): R_{T} = 4.42 (99%). |
| | | (d, J = 3.2 Hz, 3H), 2.14 (s, | |
| | | 3H), 3.60 (s, 3H), 4.05 (s, | |
| | | 3H), 4.09-4.11 (m, 1H), 5.31 | |
| | | (d, J = 10.8 Hz, 1H), 7.22 (s, | |
| | | 1H), 7.31 (m, 1H), 7.61 (s, | |
| | | 2H), 7.68 (d, J = 7.2 Hz, 1H), | |
| | | 8.83 (s, 1H), 9.30 (s, 1H), | |
| | | 10.56 (bs, 1H), 11.18 (s, 1H). | |
| Example 85 | 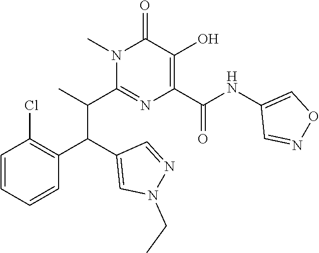 [SMILES: CCn1cc(C(c2ccccc2Cl)C(C)c2nc(C(=O)Nc3cnoc3)c(O)c(=O)n2C)cn1] | Isomer-1_ D1E1: ¹H NMR (400 MHz, DMSO-d₆): δ 1.30-1.36 (m, 6H), 3.62 (s, 3H), 4.02-4.11 (m, 3H), 5.11 (d, J = 11.2 Hz, 1H), 7.01 (t, J = 7.6 Hz, 1H), 7.16-7.21 (m, 2H), 7.55 (s, 1H), 7.63 (d, J = 7.6 Hz, 1H), 7.77 (s, 1H), 8.96 (s, 1H), 9.33 (s, 1H), 10.38 (s, 1H), 11.12 (s, 1H).Isomer-1_ D1E2: ¹H NMR (400 MHz, DMSO-d₆): ): δ 1.31-1.37 (m, 6H), 3.63 (s, 3H), 4.09-4.11 (m, 3H), 5.12 (d, J = 10.8 Hz, 1H), 7.02 (t, J = 7.6 Hz, 1H), 7.15-7.25 (m, | Isomer-1_(D1E1)_LCMS: m/z 483.32 [M⁺ + 1]. Isomer-2_(D1E2) LCMS: m/z 483.28 [M⁺ + 1]. Isomer- 3_(D2E1)_LCMS: m/z 483.30 [M⁺ + 1]. Isomer-4_(D2E2)_LCMS: m/z 483.30 [M⁺ + 1]. HPLC: FR-1 (Isomer-1; D1E1): R_{T} = 4.69 (99%); FR-2 (Isomer-2; D1E2): R_{T} = 4.70 (99%); FR-3 (Isomer-3; D2E1): R_{T} = 4.80 100%); FR-4 (Isomer-4; D2E2): R_{T} = 4.81 (99%). |
| | | 2H), 7.56 (s, 1H), 7.65 (d, | |
| | | J = 7.6 Hz, 1H), 7.78 (s, 1H), | |
| | | 8.97 (s, 1H), 9.34 (s, 1H), | |
| | | 10.41 (s, 1H), 11.14 (s, 1H). | |
| | | Isomer-1_ D2E1: ¹H NMR | |
| | | (400 MHz, DMSO-d₆): δ 1.14 | |
| | | (t, J = 7.2 Hz, 3H), 1.23 (d, | |
| | | J = 5.6 Hz, 3H), 3.39 (s, 3H), | |
| | | 3.90-3.92 (m, 3H), 4.82 (d, | |
| | | J = 10.4 Hz, 1H), 6.97 (s, 1H), | |
| | | 7.27-7.37 (m, 2H), 7.44-7.46 | |
| | | (m, 2H), 7.89 (bs, 1H), 8.95 | |
| | | (s, 1H), 9.35 (s, 1H), 10.56 (s, | |
| | | 1H), 11.42 (s, 1H). | |
| | | Isomer-1_ D2E2: ¹H NMR | |
| | | (400 MHz, DMSO-d₆): δ 1.14 | |
| | | (t, J = 7.2 Hz, 3H), 1.22 (d, | |
| | | J = 5.6 Hz, 3H), 3.39 (s, 3H), | |
| | | 3.88-3.94 (m, 3H), 4.82 (d, | |
| | | J = 10.4 Hz, 1H), 6.97 (s, 1H), | |
| | | 7.27-7.31 (m, 2H), 7.44-7.46 | |
| | | (m, 2H), 7.90 (d, J = 7.6 Hz, | |
| | | 1H), 8.95 (s, 1H), 9.35 (s, | |
| | | 1H), 10.60 (s, 1H), 11.42 (s, | |
| | | 1H). | |
| Example 153 | 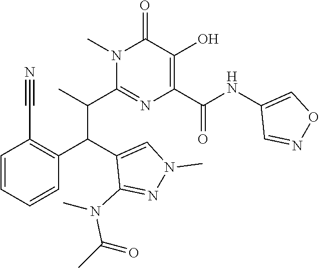 [SMILES: CC(=O)N(C)c1nn(C)cc1C(c1ccccc1C#N)C(C)c1nc(C(=O)Nc2cnoc2)c(O)c(=O)n1C] | Isomer-1_ D1E1: ¹H NMR 200 MHz, DMSO-d₆) δ 10.30 (d, 1H), 10.04 (d, 1H), 9.21 (s, 1H), 7.94 (s, 1H), 7.84 (d, 1H), 1.77-5.56 (m, 2H), 7.52-7.43 (m, 1H), 5.06 (d, 1H), 3.96 (s, 1H), 3.73 (d, 6H), 2.80-2.63 (m, 2H), 2.57 (s, 1H), 2.22 (s, 1H), 1.18 (s, 2H), 1.02 (d, 3H) 1_(D1E1) Isomer-2_D1E2: ¹H NMR (300 MHz, DMSO-d6) δ 10.30 (d, 1H), 10.04 (d, 1H), 9.21 (s, 1H), 8.78 (d, 1H), 7.94 (s, 1H), 7.84 (d, 1H), 7.77-7.56 (m, 2H), 7.52-7.43 | 1_(D1E1)_LCMS: m/z 531.3 [M⁺ + 1]. >99% ee Isomer-2_(D1E2)_LCMS: m/z 531.3 [M⁺ + 1]. >99% ee |
| | | (m, 1H), 5.06 (d, 1H), 3.96 (s, | |
| | | 1H), 3.73 (d, 6H), 2.80-2.63 | |
| | | (m, 2H), 2.57 (s, 1H), 2.22 (s, | |
| | | 1H), 1.18 (s, 2H), 1.02 (d, 3H) | |

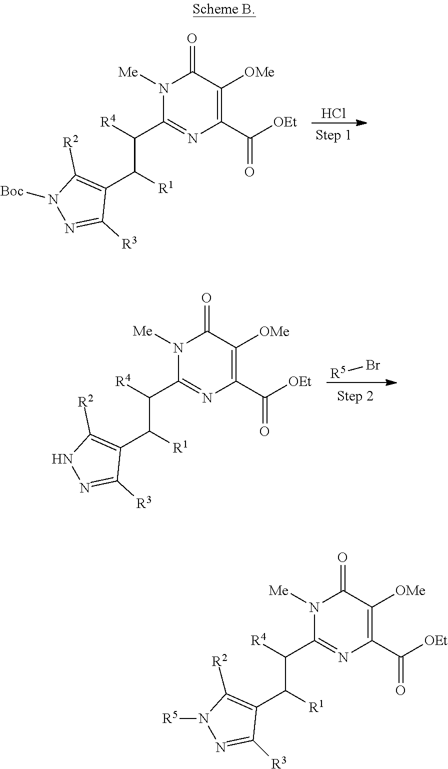

### Example 9

### Synthesis of 2-(1-(2-cyanophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide

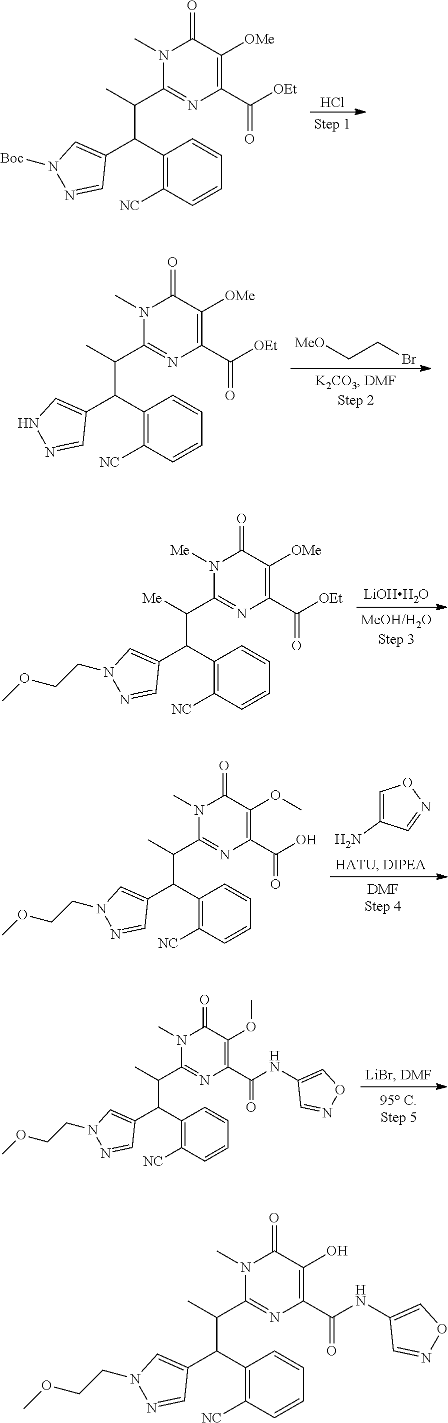

### Step 1: ethyl 2-[1-(2-cyanophenyl)-1-(1H-pyrazol-4-yl)propan-2-yl]-5-methoxy-1-methyl-6-oxopyrimidine-4-carboxylate

To a 0° C. stirred solution of 2-[1-[1-(tert-butoxycarbonyl)pyrazol-4-yl]-1-(2-cyanophenyl)propan-2-yl]-5-methoxy-1-methyl-6-oxopyrimidine-4-carboxylate) (7.00 g, 13.4 mmol) in DCM (100 mL) was added TFA (30 mL). This solution was warmed to rt and stirred for 1 h at which point it was concentrated in vacuo then cooled to 0° C. and neutralized to pH 8 with saturated NaHCO₃ (sat.). The resulting mixture was extracted with EtOAc (4×100 mL) and the combined organic layers were dried over Na₂SO₄, concentrated in vacuo, and purified by silica gel chromatography (eluting with EtOAc). Fractions containing product were collected and the solvent was removed in vacuo giving the desired product as a yellow solid (5,10 g, 90% yield).

ESI-MS m/ [symbol] : m/ [symbol] 422.2 [M+H]⁺

### Step 2: Ethyl 2-[1-(2-cyanophenyl)-1-[1-(2-methoxyethyl)pyrazol-4-yl]propan-2-yl]-5-methoxy-1-methyl-6-oxopyrimidine-4-carboxylate

To a 0° C. stirred solution of ethyl 2-[1-(2-cyanophenyl)-1-(1H-pyrazol-4-yl)propan-2-yl]-5-methoxy-1-methyl-6-oxopyrimidine-4-carboxylate (0.600 g, 1.4 mmol) in DMF (5 mL) was added 2-bromoethyl methyl ether (0.396 g, 2.85 mmol) followed by K₂CO₃ (0.590 g, 4.3 mmol). The resulting mixture was then heated to 50° C. and stirred for 3 h at which point conversion to the product was observed by LCMS. The reaction was cooled to 0° C. and quenched with water, the product was then extracted with EtOAc (3×50 mL). The combined organic layers were washed with water (3×20 mL), dried over anhydrous sodium sulphate, and concentrated in vacuo. The resulting crude reaction material was purified by silica gel column chromatography (10% EtOAc/pet. ether) fractions containing product were combined and concentrated to afford the product as a yellow solid (0.450 g, 65% yield).

ESI-MS m/ [symbol] /: m/ [symbol] 480.2 [M+H]⁺

### Step 3: 2-(2-[4-[(hydroxylithio)carbonyl]-5-methoxy-1-methyl-6-oxopyrimidin-2-yl]-1-[1-(2-methoxyethyl)pyrazol-4-yl]propyl)benzonitrile

To a solution of ethyl 2-[1-(2-cyanophenyl)-1-[1-(2-methoxyethyl)pyrazol-4-yl]propan-2-yl]-5-methoxy-1-methyl-6-oxopyrimidine-4-carboxylate (0.450 g, 0.9 mmol) dissolved in MeOH (8 mL) and water (1 mL) was added LiOH·H2O (0.079 g, 1.9 mmol) portion wise. The resulting mixture was stirred at rt for 2 h at which point it was concentrated in vacuo giving the desired product as a yellow solid (0.490 g) which was used in subsequent steps with no further purification.

ESI-MS m/ [symbol] : m/ [symbol] 452.2 [M+H]⁺

### Step 4: 2-[1-(2-cyanophenyl)-1-[1-(2-methoxyethyl)pyrazol-4-yl]propan-2-yl]-5-methoxy-1-methyl-N-(1,2-oxazol-4-yl)-6-oxopyrimidine-4-carboxamide

To a stirred solution of (2-(2-[4-[(hydroxylithio)carbonyl]-5-methoxy-1-methyl-6-oxopyrimidin-2-yl]-1-[1-(2-methoxyethyl)pyrazol-4-yl]propyl)benzonitrile) (0.500 g, 1.1 mmol) and 1,2-oxazol-4-amine hydrochloride (0.267 g, 2.2 mmol) in DMF (5 mL) was added HATU (0.842 g, 2.2 mmol) followed by DIPEA (0.286 g, 2.2 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄, and concentrated in vacuo. The resulting crude material was purified by prep-TLC (50% EtOAc/petroleum ether), the product was isolated as a yellow solid (0.481 g, 84% yield).

ESI-MS m/ [symbol] : 517.2 [M+H]⁺

Separation of diastereomers was done at this step using reverse phase C18 chromatography: Column Ultimate XB-C18 Column, 16 um, 50*250 mm; 15% to 55% acetonitrile/water (0.1% FA) in 45 min; Flow rate: 65 mL/min.

Peak 1_D1 contained 160 mg of a white solid.

Peak 2_D2 Contained 195 mg of a white solid.

Enantiomers of this material were separated by Prep-chiral-HPLC:

D1: Column: NB-Lux 5 um i-Cellulose-5, 2.12*25 cm, 5 m; Mobile Phase A: Hex:MTBE=1:1(0.5% 2M NH3-MEOH), Mobile Phase B:EtOH-HPLC; Flow rate: 20 mL/min; Gradient: 30% B to 30% B in 13.5 min

Peak 1 (Isomer-1_D1E1): R_{T} 9.69 min; afforded a white solid (50 mg)

Peak 2 (Isomer-2_D1E2): R_{T} 11.45 min; afforded a white solid (47 mg)

D2: Column: CHIRALPAK IA, 2*25 cm, Sum; Hex:MTBE=1:1(0.5% 2M NH3-MEOH), Mobile Phase B:EtOH-HPLC; Flow rate:20 mL/min; Gradient: 20% B to 20% B in 10 min).

Peak 1 (Isomer-3_D2E1): R_{T} 6.68 min; afforded a white solid (70 mg)

Peak 2 (Isomer-4_D2E2): R_{T} 8.25 min; afforded a white solid (68 mg)

### Scheme B, Step 5: 2-[1-(2-cyanophenyl)-1-[1-(2-methoxyethyl)pyrazol-4-yl]propan-2-yl]-5-hydroxy-1-methyl-N-(1,2-oxazol-4-yl)-6-oxopyrimidine-4-carboxamide

To a solution of 2-[1-(2-cyanophenyl)-1-[1-(2-methoxyethyl)pyrazol-4-yl]propan-2-yl]-5-methoxy-1-methyl-N-(1,2-oxazol-4-yl)-6-oxopyrimidine-4-carboxamide (0.050 mg, 0.1 mmol) dissolved in DMF (3 ml) was added LiBr (0.017 mg, 0.2 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1 Isolated product as a white solid (0.026 g, 54% yield)

ESI-MS m/ [symbol] : 504.3 [M+H]⁺; >98% ee

¹H NMR (400 MHz, DMSO-d6): δ 11.24 (brs, 1H), 10.63 (brs, 1H), 9.28 (s, 1H), 8.86 (s, 1H), 7.82-7.78 (m, 2H), 7.62-7.54 (m, 3H), 7.21 (t, J=7.6 Hz, 1H), 4.99 (d, J=11.0 Hz, 1H), 4.22 (t, J=5.3 Hz, 2H), 4.09-4.05 (m, 1H), 3.76 (t, J=5.3 Hz, 2H), 3.65 (s, 3H), 3.19 (s, 3H), 1.31 (d, J=6.5 Hz, 3H).

Isomer-2_D1E2: Isolated product as a white solid (0.028 g, 61% yield)

ESI-MS m/ [symbol] : 504.3 [M+H]⁺; >95% ee

¹H NMR (400 MHz, DMSO-d6): δ 11.25 (brs, 1H), 10.60 (brs, 1H), 9.28 (s, 1H), 8.86 (s, 1H), 7.82-7.78 (m, 2H), 7.62-7.53 (m, 3H), 7.21 (t, J=7.6 Hz, 1H), 5.00 (d, J=11.0 Hz, 1H), 4.22 (t, J=5.3 Hz, 2H), 4.09-4.05 (m, 1H), 3.66 (t, J=5.3 Hz, 2H), 3.59 (s, 3H), 3.19 (s, 3H), 1.31 (d, J=6.5 Hz, 3H).

Isomer-3_D2E1: Isolated an off-white solid (0.051 g, 78% yield)

ESI-MS m/ [symbol] : 504.3 [M+H]⁺; >98% ee

¹H NMR (400 MHz, methanol-d4): δ 11.24 (s, 1H), 10.46 (brs, 1H), 9.36 (s, 1H), 8.89 (s, 1H), 8.00 (d, J=7.9 Hz, 1H), 7.97-7.78 (m, 2H), 7.50 (t, J=7.6, 1.1 Hz, 1H), 7.47 (s, 1H), 7.37 (s, 1H), 4.85 (d, J=10.6 Hz, 1H), 4.07-4.04 (m, 3H), 3.49-3.43 (m, 5H), 3.05 (s, 3H), 1.18 (d, J=6.5 Hz, 3H).

Isomer-4_D2E2: Isolated an off-white solid (0.051 g, 78% Yield)

ESI-MS m/ [symbol] : 504.3 [M+H]⁺; >95% ee

¹H NMR (400 MHz, methanol-d4): δ 11.24 (s, 1H), 10.49 (brs, 1H), 9.33 (s, 1H), 8.88 (s, 1H), 7.98 (d, J=7.9 Hz, 1H), 7.85-7.78 (s, 2H), 7.50 (t, J=7.6, 1.1 Hz, 1H), 7.46 (s, 1H), 7.10 (s, 1H), 4.85 (d, J=10.6 Hz, 1H), 4.07-4.04 (s, 3H), 3.49-3.43 (m, 5H), 3.05 (s, 3H), 1.18 (d, J=6.6.5 Hz, 3H).

The following compounds in Table 3 were prepared according to the Scheme B methods described above.

TABLE 3

| | | | ESI-MS m/ [symbol] |
|---|---|---|---|
| ID | Structure | ¹H NMR | [M + H] |
| Example 10 | 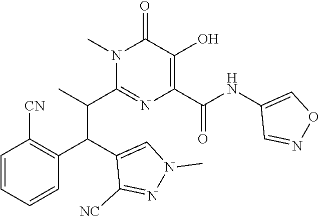 [SMILES: CC(c1nc(C(=O)Nc2cnoc2)c(O)c(=O)n1C)C(c1ccccc1C#N)c1cn(C)nc1C#N] | Isomer-1_D1E1: ¹H NMR (400 MHz, DMSO-d₆) δ 11.30 (s, 1H), 10.44 (s, 1H), 9.30 (s, 1H), 8.85 (s, 1H), 8.36 (s, 1H), 7.74-7.54 (m, 3H), 7.28 (m, 1H), 5.04 (d, J = 10.9 Hz, 1H), 4.13 (m, 1H), 3.97 (s, 3H), 3.53 (s, 3H), 1.43 (d, J = 6.5 Hz, 3H). Isomer-2_D1E2: ¹H NMR (400 MHz, DMSO-d₆) δ 11.30 (s, 1H), 10.44 (s, 1H), 9.30 (s, 1H), 8.85 (s, 1H), 8.36 (s, 1H), 7.74-7.54 (m, | Isomer-1_D1E1: m/ [symbol] 485.1 [M + H] >98% ee Isomer-2_D1E2: m/ [symbol] 485.1 [M + H] >98% ee Isomer-3_D2E1: m/ [symbol] 485.1 [M + H] >98% ee Isomer-4_D2E2: m/ [symbol] 485.1 [M + H] >98% ee |
| | | 3H), 7.28 (m, 1H), 5.04 (d, | |
| | | J = 10.9 Hz, 1H), 4.13 (m, | |
| | | 1H), 3.97 (s, 3H), 3.53 (s, | |
| | | 3H), 1.43 (d, J = 6.5 Hz, 3H). | |
| Example 11 | 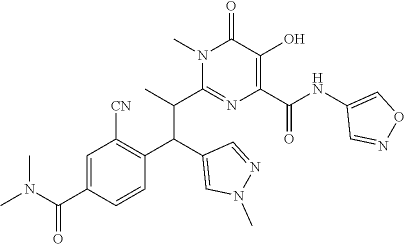 [SMILES: CC(c1nc(C(=O)Nc2cnoc2)c(O)c(=O)n1C)C(c1cnn(C)c1)c1ccc(C(=O)N(C)C)cc1C#N] | Isomer-1_D1E1: ¹H NMR (400 MHz, DMSO-d₆): δ 11.18 (s, 1H), 10.45 (s, 1H), 9.29 (s, 1H), 8.87 (s, 1H), 7.85 (d, 1H), 7.82 (s, 1H), 7.65 (d, 1H), 7.62 (d, 1H), 7.58-7.56 (m, 1H), 5.00 (d, 1H), 4.12-4.05 (m, 1H), 3.81 (s, 3H), 3.60 (s, 3H), 2.89 (s, 3H), 2.71 (s, 3H), 1.34 (d, 3H). Isomer-2_D1E2: ¹H NMR (400 MHz, DMSO-d₆): δ 11.18 (s, 1H), 10.45 (s, 1H), 9.29 (s, 1H), 8.87 (s, 1H), | Isomer-1_D1E1: m/ [symbol] 531.4. [M + H] >98% ee Isomer-2_D1E2: m/ [symbol] 531.4. [M + H] >98% ee Isomer-3_D2E1: m/ [symbol] 531.4. [M + H] >96% ee Isomer-4_D2E2: m/ [symbol] 531.4. [M + H] >93% ee |
| | | 7.84 (d, 1H), 7.82 (s, 1H), | |
| | | 7.65 (d, 1H), 7.62 (d, 1H), | |
| | | 7.58-7.56 (m, 1H), 5.00 (d, | |
| | | 1H), 4.12 − 4.05 (m, 1H), | |
| | | 3.81 (s, 3H), 3.60 (s, 3H), | |
| | | 2.89 (s, 3H), 2.71 (s, 3H), | |
| | | 1.35 (d, 3H). | |
| | | Isomer-3_D2E1: ¹H NMR | |
| | | (400 MHz, DMSO-d₆): δ | |
| | | 11.22 (s, 1H), 10.37 (s, 1H), | |
| | | 9.33 (s, 1H), 8.86 (s, 1H), | |
| | | 7.99 (d, 1H), 7.88 (d, 1H), | |
| | | 7.81 (d, 1H), 7.44 (s, 1H), | |
| | | 7.16 (s, 1H), 4.91 (d, 1H), | |
| | | 4.09-4.03 (m, 1H), 3.66 (s, | |
| | | 3H), 3.51 (s, 3H), 2.99 (s, | |
| | | 3H), 2.90 (s, 3H), 1.17 (d, , | |
| | | 3H). | |
| | | Isomer-4_D2E2: ¹H NMR | |
| | | (400 MHz, DMSO-d₆): δ | |
| | | 11.22 (s, 1H), 10.36 (s, 1H), | |
| | | 9.33 (s, 1H), 8.87 (s, 1H), | |
| | | 7.99 (d, 1H), 7.88 (d, 1H), | |
| | | 7.81 (d, 1H), 7.44 (s, 1H), | |
| | | 7.16 (s, 1H), 4.91 (d, 1H), | |
| | | 4.09-4.05 (m, 1H), 3.66 (s, | |
| | | 3H), 3.51 (s, 3H), 2.99 (s, | |
| | | 3H), 2.90 (s, 3H), 1.17 (d, | |
| | | 3H). | |
| Example 12 | 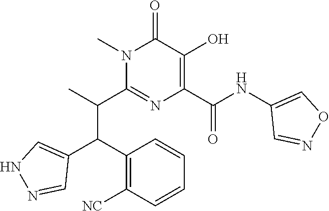 [SMILES: CC(c1nc(C(=O)Nc2cnoc2)c(O)c(=O)n1C)C(c1cn[nH]c1)c1ccccc1C#N] | Isomer-1_D1E1: ¹H NMR (400 MHz, DMSO-d₆) δ 12.74 (brs, 1H), 11.39 9.29 (s, 1H), 8.87 (s, 1H), (brs,1H), 10.67 (brs, 1H), 7.74 (s, 3H), 7.61 − 7.51 (m, 2H), 7.20 (t, 1H), 5.02 (d, 1H), 4.13-4.07 (m, 1H), 3.60 (s, 3H), 1.32 (d, 3H). Isomer-2_D1E2: ¹H NMR (400 MHz, DMSO-d₆): δ 12.74 (s, 1H), 11.39 (s,1H), 10.67 (s,1H), 9.29 (s, 1H), | |
| | | 8.87 (s, 1H), 7.74 (s, 3H), | |
| | | 7.61 − 7.51 (m, 2H), 7.20 (t, | |
| | | 1H), 5.02 (d, 1H), 4.13-4.07 | |
| | | (m, 1H), 3.60 (s, 3H), 1.31 | |
| | | (d, 3H) | |
| | | Isomer-3_D2E1: ¹H NMR | |
| | | (400 MHz, DMSO-d₆): δ | |
| | | 12.57 (s, 1H), 11.21 (s, 1H), | |
| | | 10.42 (s, 1H), 9.33 (s, 1H), | |
| | | 8.88 (s, 1H), 7.98 (d, 1H), | |
| | | 7.86 − 7.75 (m, 2H), 7.47 (t, | |
| | | 1H), 7.41 (s, 1H), 7.19 (s, | |
| | | 1H), 4.88 (d, 1H), 4.07-4.02 | |
| | | (m, 1H), 3.46 (s, 3H), 1.17 | |
| | | (d, 3H) | |
| | | Isomer-4_D2E2: ¹H NMR | |
| | | (400 MHz, DMSO-d₆): δ | |
| | | 12.57 (s, 1H), 11.21 (s, 1H), | |
| | | 10.42 (s, 1H), 9.33 (s, 1H), | |
| | | 8.87 (s, 1H), 7.98 (d, 1H), | |
| | | 7.86 − 7.75 (m, 2H), 7.47 (t, | |
| | | 1H), 7.40 (s, 1H), 7.20 (s, | |
| | | 1H), 4.88 (d, 1H), 4.07-4.02 | |
| | | (m, 1H), 3.46 (s, 3H), 1.17 | |
| | | (d, 3H). | |
| Example 13 | 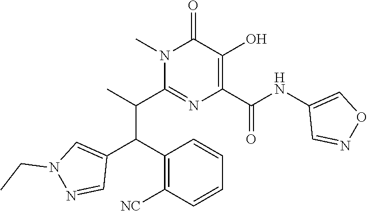 [SMILES: CCn1cc(C(c2ccccc2C#N)C(C)c2nc(C(=O)Nc3cnoc3)c(O)c(=O)n2C)cn1] | Isomer-1_D1E1: ¹H NMR (400 MHz, DMSO-d₆): δ 11.19 (s, 1H), 10.55 (s, 1H), 9.29 (s, 1H), 8.87 (s, 1H), 7.81 (d, J = 13.1 Hz, 2H), 7.65 − 7.50 (m, 3H), 7.21 (t, J = 7.5 Hz, 1H), 4.99 (d, J = 11.0 Hz, 1H), 4.13 − 4.03 (m, 3H), 3.60 (s, 3H), 1.44 − 1.24 (m, 6H Isomer-2_D1E2: ¹H NMR (400 MHz, DMSO-d₆): δ 11.18 (s, 1H), 10.44 (s, 1H), | Isomer-1_D1E1: m/ [symbol] 474.4. [M + H] Isomer-2_D1E2: m/ [symbol] 474.3. [M + H] Isomer-3_D2E1: m/ [symbol] 474.4. [M + H] Isomer-4_D2E2: m/ [symbol] 474.4. [M + H] |
| | | 9.29 (s, 1H), 8.87 (s, 1H), | |
| | | 7.81 (d, J = 13.4 Hz, 2H), | |
| | | 7.62 − 7.52 (m, 3H), 7.21 (td, | |
| | | J = 7.7, 1.1 Hz, 1H), 4.98 (d, | |
| | | J = 11.0 Hz, 1H), 4.10 (q, J = | |
| | | 7.4 Hz, 3H), 3.60 (s, 3H), | |
| | | 1.39 − 1.29 (m, 6H) | |
| | | Isomer-3_D2E1: ¹H NMR | |
| | | (400 MHz, DMSO-d₆): δ | |
| | | 11.26 (s, 1H), 10.35 (s, | |
| | | 1H), 9.33 (s, 1H), 8.88 (s, | |
| | | 1H), 7.97 (d, J = 7.9 Hz, 1H), | |
| | | 7.87 − 7.74 (m, 2H), 7.51 − | |
| | | 7.44 (m, 1H), 7.39 (s, 1H), | |
| | | 7.08 (s, 1H), 4.81 (d, J = 10.4 | |
| | | Hz, 1H), 4.03 (s, 1H), 3.94 | |
| | | (q, J = 7.2 Hz, 2H), 3.44 (s, | |
| | | 3H), 1.18-1.11 (m, 6H | |
| | | Isomer-4_D2E2: ¹H NMR | |
| | | (400 MHz, DMSO-d₆) δ | |
| | | 11.26 (s, 1H), 10.49(s, 1H) | |
| | | 9.33 (s, 1H), 8.89 (s, 1H), | |
| | | 7.97 (d, J = 7.9 Hz, 1H), 7.87 − | |
| | | 7.75 (m, 2H), 7.48 (td, J = | |
| | | 7.6, 1.1 Hz, 1H), 7.39 (s, | |
| | | 1H), 7.09 (s, 1H), 4.81 (d, J = | |
| | | 10.4 Hz, 1H), 4.03 (dd, J = | |
| | | 10.5, 6.4 Hz, 1H), 3.94 (q, | |
| | | J = 7.2 Hz, 2H), 3.44 (s, 3H), | |
| | | 1.18-1.11 (m, 6H) | |
| Example 14 | 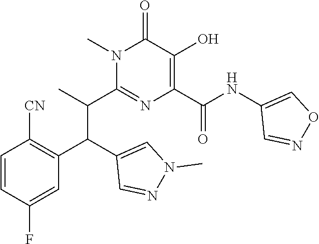 [SMILES: CC(c1nc(C(=O)Nc2cnoc2)c(O)c(=O)n1C)C(c1cnn(C)c1)c1cc(F)ccc1C#N] | Isomer-1_D1E1: ¹H NMR (300 MHz, DMSO-d₆): δ 11.18 (s, 1H), 10.43 (s, 1H), 9.30 (s, 1H), 8.86 (s, 1H), 7.81 (s, 1H), 7.80-7.75 (m, 1H), 7.75-7.69 (m, 1H), 7.62 (d, J = 0.8 Hz, 1H), 7.14- 7.05 (m, 1H), 5.06-5.00 (m, 1H), 4.12-4.04 (m, 1H), 3.82 (s, 3H), 3.64 (s, 3H), 1.31 (d, J = 6.5 Hz, 3H). Isomer-2_D1E2: ¹H NMR (300 MHz, DMSO-d₆): δ 11.18 (s, 1H), 10.44 (s, 1H), 9.30 (s, 1H), 8.86 (s, 1H), | Isomer-1_D1E1: m/ [symbol] 477.9. [M + H] >98% ee Isomer-2_D1E2: m/ [symbol] 477.9. [M + H] >98% ee Isomer-3_D2E1: m/ [symbol] 478.0. [M + H] >98% ee Isomer-4_D2E2: m/ [symbol] 478.0. [M + H] >98% ee |
| | | 7.81 (s, 1H), 7.80-7.75 (m, | |
| | | 1H), 7.75-7.69 (m, 1H), 7.62 | |
| | | (d, J = 0.8 Hz, 1H), 7.13- | |
| | | 7.05 (m, 1H), 5.06-5.00 (m, | |
| | | 1H), 4.14 − 4.02 (m, 1H), | |
| | | 3.82 (s, 3H), 3.64 (s, 3H), | |
| | | 1.31 (d, J = 6.5 Hz, 3H). | |
| | | Isomer-3_D2E1: ¹H NMR | |
| | | (300 MHz, DMSO-d₆): δ | |
| | | 11.19 (s, 1H), 10.46 (brs, | |
| | | 1H), 9.33 (s, 1H), 8.87 (s, | |
| | | 1H), 7.99-7.88 (m, 2H), 7.43 | |
| | | (s, 1H), 7.40-7.33 (m, 1H), | |
| | | 7.15 (s, 1H), 4.90 (d, J = 10.4 | |
| | | Hz, 1H), 4.11-4.03 (m, 1H), | |
| | | 3.66 (s, 3H), 3.50 (s, 3H), | |
| | | 1.18 (d, J = 6.5 Hz, 3H). | |
| | | Isomer-4_D2E2: ¹H NMR | |
| | | (300 MHz, DMSO-d₆): δ | |
| | | 11.19 (s, 1H), 9.33 (s, 1H), | |
| | | 8.86 (s, 1H), 7.99 − 7.86 (m, | |
| | | 2H), 7.43 (s, 1H), 7.40 − 7.32 | |
| | | (m, 1H), 7.15 (s, 1H), 4.89 | |
| | | (d, J = 10.4 Hz, 1H), 4.06 (s, | |
| | | 1H), 3.66 (s, 3H), 3.50 (s, | |
| | | 3H), 1.18 (d, J = 6.6 Hz, 3H). | |
| Example 15 | 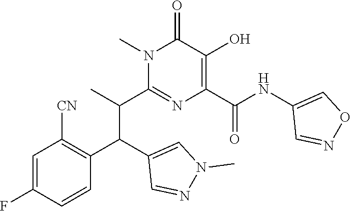 [SMILES: CC(c1nc(C(=O)Nc2cnoc2)c(O)c(=O)n1C)C(c1cnn(C)c1)c1ccc(F)cc1C#N] | Isomer-1_D1E1: ¹H NMR (400 MHz, DMSO-d₆): δ 11.20 (brs, 1H), 10.67 (brs, 1H), 9.28 (s, 1H), 8.85 (s, 1H), 7.83 (m, 1H), 7.77 (s, 1H), 7.60-7.58 (m, 2H), 7.45 (t, 1H), 4.98 (d, 1H), 4.07- 4.03 (m, 1H), 3.81 (s, 3H), 3.60 (s, 3H), 1.33 (d, 3H). Isomer-2_D1E2: ¹H NMR (400 MHz, DMSO-d₆): δ 11.22 (brs, 1H), 10.56 (brs, 1H), 9.29 (s, 1H), 8.86 (s, | Isomer-1_D1E1: m/ [symbol] 478.1. [M + H] Isomer-2_D1E2: m/ [symbol] 478.1. [M + H] Isomer-3_D2E1: m/ [symbol] 478.1. [M + H] Isomer-4_D2E2: m/ [symbol] 478.1. [M + H] |
| | | 1H), 7.85 (m, 1H), 7.78 (s, | |
| | | 1H), 7.62-7.58 (d, 2H), 7.45 | |
| | | (t, 1H), 4.99 (d, 1H), 4.08- | |
| | | 4.03 (m, 1H), 3.81 (s, 3H), | |
| | | 3.60 (s, 3H), 1.32 (d, 3H). | |
| | | Isomer-3_D2E1: ¹H NMR | |
| | | (400 MHz, DMSO-d₆): δ | |
| | | 11.22 (s, 1H), 10.49 (s, 1H), | |
| | | 9.32 (s, 1H), 8.85 (s, 1H), | |
| | | 7.97 (m, 1H), 7.84 (m, 1H), | |
| | | 7.69 (m, 1H), 7.39 (s, 1H), | |
| | | 7.12 (s, 1H), 4.88 (d, 1H), | |
| | | 4.04-3.98 (m, 1H), 3.65 (s, | |
| | | 3H), 3.49 (s, 3H), 1.14 (d, | |
| | | 3H). | |
| | | Isomer-4_D2E2: ¹H NMR | |
| | | (400 MHz, DMSO-d₆): δ | |
| | | 11.21 (brs, 1H),10.62 (brs, | |
| | | 1H) 9.31 (s, 1H), 8.85 (s, | |
| | | 1H), 7.97 (m, 1H), 7.83 (m, | |
| | | 1H), 7.69 − 7.66 (m, 1H), | |
| | | 7.39 (s, 1H), 7.12 (s, 1H), | |
| | | 4.88 (d, 1H), 4.03-3.98 (m, | |
| | | 1H), 3.65 (s, 3H), 3.49 (s, | |
| | | 3H), 1.13 (d, 3H). | |
| Example 16 | 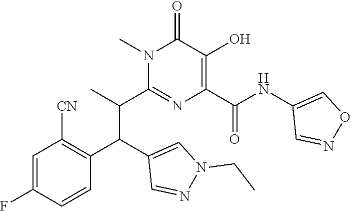 [SMILES: CCn1cc(C(c2ccc(F)cc2C#N)C(C)c2nc(C(=O)Nc3cnoc3)c(O)c(=O)n2C)cn1] | Isomer-1_D1E1: ¹H NMR (400 MHz, DMSO-d₆) δ 11.19 (s, 1H), 10.43 (s, 1H), 9.30 (s, 1H), 8.86 (s, 1H), 7.88-7.84 (m, 1H), 7.82 (s, 1H), 7.61 − 7.58 (m, 2H), 7.48-7.43 (m, 1H), 4.99 (d, 1H), 4.13 − 4.04 (m, 3H), 3.60 (s, 3H), 1.37-1.32(m, 6H). Isomer-2_D1E2: ¹H NMR (400 MHz, DMSO-d₆) δ 11.20 (s, 1H), 10.47 (s, 1H), | Isomer-1_D1E1: m/ [symbol] 492.2. [M + H] >98% ee Isomer-2_D1E2: m/ [symbol] 492.2. [M + H] >98% ee Isomer-3_D2E1: m/ [symbol] 492.2. [M + H] >98% ee Isomer-4_D2E2: m/ [symbol] 492.4. [M + H] >98% ee |
| | | 9.30 (s, 1H), 8.86 (s, 1H), | |
| | | 7.88-7.83 (m, 2H), 7.61 − | |
| | | 7.59 (m, 2H), 7.48-7.43 (m, | |
| | | 1H), 4.99 (d, 1H), 4.13 − | |
| | | 4.07 (m, 3H), 3.60 (s, 3H), | |
| | | 1.37-1.31(m, 6H). | |
| | | Isomer-3_D2E1: ¹H NMR | |
| | | (400 MHz, DMSO-d₆) δ | |
| | | 11.26 (s, 1H), 10.44 (s, 1H), | |
| | | 9.33 (s, 1H), 8.87 (s, 1H), | |
| | | 8.13-7.99 (m, 1H), 7.86-7.86 | |
| | | (m, 1H), 7.72-7.68 (m, 1H), | |
| | | 7.39 (s, 1H), 7.09 (s, 1H), | |
| | | 4.80 (d, 1H), 4.04-3.92 (m, | |
| | | 3H), 3.44 (s, 3H), 1.19 − 1.15 | |
| | | (m, 6H). | |
| | | Isomer-4_D2E2: ¹H NMR | |
| | | (400 MHz, DMSO-d₆) δ | |
| | | 11.29 (s, 1H), 10.48 (s, 1H), | |
| | | 9.33 (s, 1H), 8.87 (s, 1H), | |
| | | 8.13-7.99 (m, 1H), 7.86-7.83 | |
| | | (m, 1H), 7.73-7.68 (m, 1H), | |
| | | 7.39 (s, 1H), 7.09 (s, 1H), | |
| | | 4.80 (d, 1H), 4.03-3.92 (m, | |
| | | 3H), 3.44 (s, 3H), 1.19 − 1.15 | |
| | | (m, 6H). | |
| Example 17 | 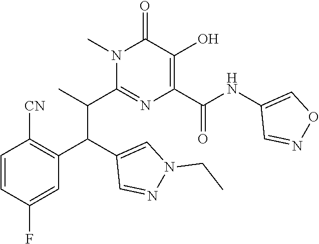 [SMILES: CCn1cc(C(c2cc(F)ccc2C#N)C(C)c2nc(C(=O)Nc3cnoc3)c(O)c(=O)n2C)cn1] | Isomer-1_D1E1: ¹H NMR (400 MHz, DMSO-d₆) δ 11.19 (s, 1H), 10.45 (s, 1H), 9.30 (s, 1H), 8.86 (s, 1H), 7.86 (s, 1H), 7.80-7.77 (m, 1H), 7.74 − 7.70 (m, 1H), 7.63 (s, 1H), 7.11-7.07 (m, 1H), 5.04-5.01 (d, 1H), 4.14- 4.07 (m, 3H), 3.63 (s, 3H), 1.37-1.29 (m, 6H). Isomer-2_D1E2: ¹H NMR (400 MHz, DMSO-d₆) δ 11.19 (s, 1H), 10.45 (s, 1H), 9.30 (s, 1H), 8.86 (s, 1H), 7.86 (s, 1H), 7.80-7.77 (m, | Isomer-1_D1E1: m/ [symbol] 492.4. [M + H] >98% ee Isomer-2_D1E2: m/ [symbol] 492.4. [M + H] >98% ee Isomer-3_D2E1: m/ [symbol] 492.4. [M + H] >98% ee Isomer-4_D2E2: m/ [symbol] 492.4. [M + H] >98% ee |
| | | 1H), 7.74 − 7.70 (m, 1H), | |
| | | 7.63 (s, 1H), 7.11-7.07 (m, | |
| | | 1H), 5.04-5.01 (d, 1H), 4.14- | |
| | | 4.07 (m, 3H), 3.63 (s, 3H), | |
| | | 1.37-1.29 (m, 6H). | |
| | | Isomer-3_D2E1: ¹H NMR | |
| | | (400 MHz, DMSO-d₆) δ | |
| | | 11.25 (s, 1H), 10.43 (s, 1H), | |
| | | 9.34(s, 1H), 8.89 (s, 1H), | |
| | | 7.97-7.92 (m, 2H), 7.43 (s, | |
| | | 1H), 7.40 − 7.35 (m, 1H), | |
| | | 7.12 (s, 1H), 4.83-4.80 (d, | |
| | | 1H), 4.08-4.04 (m, 1H), | |
| | | 3.97-3.92 (m, 2H), 3.44 (s, | |
| | | 3H), 1.22-1.15 (m, 6H). | |
| | | Isomer-4_D2E2: ¹H NMR | |
| | | (400 MHz, DMSO-d₆) δ | |
| | | 11.25 (s, 1H), 10.43 (s, 1H), | |
| | | 9.34(s, 1H), 8.89 (s, 1H), | |
| | | 7.97-7.92 (m, 2H), 7.43 (s, | |
| | | 1H), 7.40 − 7.35 (m, 1H), | |
| | | 7.12 (s, 1H), 4.83-4.80 (d, | |
| | | 1H), 4.08-4.04 (m, 1H), | |
| | | 3.97-3.92 (m, 2H), 3.44 (s, | |
| | | 3H), 1.22-1.15 (m, 6H). | |
| Example 18 | 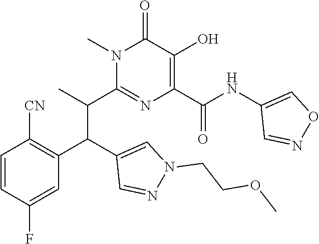 [SMILES: COCCn1cc(C(c2cc(F)ccc2C#N)C(C)c2nc(C(=O)Nc3cnoc3)c(O)c(=O)n2C)cn1] | Isomer-1_D1E1: ¹H NMR (400 MHz, DMSO-d₆) δ 11.19 (s, 1H), 10.47 (s, 1H), 9.30 (s, 1H), 8.86 (s, 1H), 7.84 (s, 1H), 7.81-7.78 (m, 1H), 7.74 − 7.70 (m, 1H), 7.66 (s, 1H), 7.12-7.07 (m, 1H), 5.05-5.03 (d, 1H), 4.25- 4.22 (m, 2H), 4.09-4.08 (m, 1H), 3.67-3.63 (m, 5H), 3.20 (s, 3H), 1.30-1.29 (m, 3H). Isomer-2_D1E2: ¹H NMR (400 MHz, DMSO-d₆) δ 11.19 (s, 1H), 10.47 (s, 1H), 9.30 (s, 1H), 8.86 (s, 1H), | Isomer-1_D1E1: m/ [symbol] 522.2. [M + H] >98% ee Isomer-2_D1E2: m/ [symbol] 522.2. [M + H] >98% ee Isomer-3_D2E1: m/ [symbol] 522.2. [M + H] >98% ee Isomer-4_D2E2: m/ [symbol] 522.2. [M + H] >98% ee |
| | | 7.84 (s, 1H), 7.81-7.78 (m, | |
| | | 1H), 7.74 − 7.70 (m, 1H), | |
| | | 7.66 (s, 1H), 7.12-7.07 (m, | |
| | | 1H), 5.05-5.03 (d, 1H), 4.25- | |
| | | 4.22 (m, 2H), 4.09-4.08 (m, | |
| | | 1H), 3.67-3.63 (m, 5H), 3.20 | |
| | | (s, 3H), 1.30-1.29 (m, 3H). | |
| | | Isomer-3_D2E1: ¹H NMR | |
| | | (400 MHz, DMSO-d₆) δ | |
| | | 11.23 (s, 1H), 10.45 (s, 1H), | |
| | | 9.34 (s, 1H), 8.88 (s, 1H), | |
| | | 7.98-7.94 (m, 2H), 7.42-7.34 | |
| | | (m, 2H), 7.14 (s, 1H), 4.86- | |
| | | 4.83 (d, 1H), 4.08-4.05 (m, | |
| | | 3H), 4.48-4.46 (m, 5H), 3.05 | |
| | | (s, 3H), 1.21-1.20 (m, 3H). | |
| | | Isomer-4_D2E2: ¹H NMR | |
| | | (400 MHz, DMSO-d₆) δ | |
| | | 11.23 (s, 1H), 10.45 (s, 1H), | |
| | | 9.34 (s, 1H), 8.88 (s, 1H), | |
| | | 7.98-7.94 (m, 2H), 7.42-7.34 | |
| | | (m, 2H), 7.14 (s, 1H), 4.86- | |
| | | 4.83 (d, 1H), 4.08-4.05 (m, | |
| | | 3H), 4.48-4.46 (m, 5H), 3.05 | |
| | | (s, 3H), 1.21-1.20 (m, 3H). | |
| Example 19 | 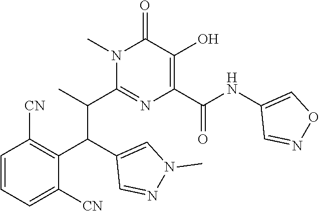 [SMILES: CC(c1nc(C(=O)Nc2cnoc2)c(O)c(=O)n1C)C(c1cnn(C)c1)c1c(C#N)cccc1C#N] | Isomer-1_D1E1: ¹H NMR (400 MHz, DMSO-d₆): δ 11.06 (s, 1H), 10.38 (s, 1H), 9.29 (s, 1H), 8.82 (s, 1H), 8.04 − 8.02 (d, 2H), 7.85 (s, 1H), 7.58 (s, 1H), 7.46-7.42 (m, 1H), 5.26-5.23 (d, 1H), 4.52-4.48 (m, 1H), 3.83 (s, 3H), 3.67 (s, 3H), 1.49-1.47 (d, 3H). Isomer-2_D1E2: ¹H NMR (400 MHz, DMSO-d₆): δ 11.06 (s, 1H), 10.38 (s, 1H), | Isomer-1_D1E1: m/ [symbol] 485.2. [M + H] Isomer-2_D1E2: m/ [symbol] 485.2. [M + H] Isomer-3_D2E1: m/ [symbol] 485.2. [M + H] Isomer-4_D2E2: m/ [symbol] 485.2. [M + H] |
| | | 9.29 (s, 1H), 8.82 (s, 1H), | |
| | | 8.04 − 8.02 (d, 2H), 7.85 (s, | |
| | | 1H), 7.58 (s, 1H), 7.46-7.42 | |
| | | (m, 1H), 5.26-5.23 (d, 1H), | |
| | | 4.52-4.48 (m, 1H), 3.83 (s, | |
| | | 3H), 3.67 (s, 3H), 1.49-1.47 | |
| | | (d, 3H). | |
| | | Isomer-3_D2E1: ¹H NMR | |
| | | (400 MHz, DMSO-d₆): δ | |
| | | 11.12 (s, 1H), 10.14-10.12 | |
| | | (m, 1H), 9.30 (s, 1H), 8.77 | |
| | | (s, 1H), 8.30 − 8.28 (d, 2H), | |
| | | 7.77-7.72 (m, 1H), 7.44 (s, | |
| | | 1H), 7.05 (m, 1H), 5.46-5.43 | |
| | | (d, 1H), 4.33-4.28 (m, 1H), | |
| | | 3.71 (s, 3H), 3.60 (s, 3H), | |
| | | 1.16-1.13 (d, 3H). | |
| | | Isomer-4_D2E2: ¹H NMR | |
| | | (400 MHz, DMSO-d₆): δ | |
| | | 11.12 (s, 1H), 10.43-10.38 | |
| | | (m, 1H), 9.30 (s, 1H), 8.77 | |
| | | (s, 1H), 8.30 − 8.28 (d, 2H), | |
| | | 7.77-7.72 (m, 1H), 7.43 (s, | |
| | | 1H), 7.03 (m, 1H), 5.45-5.41 | |
| | | (d, 1H), 4.33-4.28 (m, 1H), | |
| | | 3.71 (s, 3H), 3.60 (s, 3H), | |
| | | 1.24-1.12 (d, 3H). | |
| Example 20 | 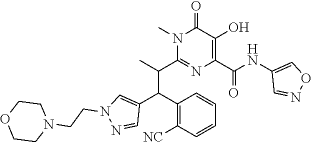 | Isomer-1_D1E1: ¹H NMR (400 MHz, Chloroform-d): δ 11.67 (s, 1H), 9.44 (s, 1H), 9.15 (s, 1H), 8.82 (s, 1H), 7.66 − 7.49 (m, 4H), 7.43 (d, J = 7.9 Hz, 1H), 7.24 (dd, J = 7.7, 1.1 Hz, 1H), 5.06 (d, J = 11.2 Hz, 1H), 4.42-4.38 (s, 2H), 3.72 (d, J = 14.5 Hz, | Isomer-1_D1E1: m/ [symbol] 595.3. [M + H] Isomer-2_D1E2: m/ [symbol] 595.3. [M + H] Isomer-3_D2E1: m/ [symbol] 595.3. [M + H] Isomer-4_D2E2: m/ [symbol] 595.3. [M + H] |
| | | 8H), 3.12-3.02 (m, 2H), | |
| | | 2.683.56 (m, 4H), 1.35 (d, | |
| | | J = 6.6 Hz, 3H). | |
| | | Isomer-2_D1E2: ¹H NMR | |
| | | (400 MHz, Chloroform-d): δ | |
| | | 11.67 (s, 1H), 9.45 (s, 1H), | |
| | | 9.15 (s, 1H), 8.82 (s, 1H), | |
| | | 7.63 (s, 1H), 7.58 − 7.50 (m, | |
| | | 3H), 7.44 (d, J = 7.9 Hz, 1H), | |
| | | 7.24 (dd, J = 7.6, 1.1 Hz, | |
| | | 1H), 5.05 (d, J = 11.2 Hz, | |
| | | 1H), 4.45 (s, 2H), 3.83 − 3.72 | |
| | | (m, 5H), 3.70 (s, 3H), 3.16 | |
| | | (s, 2H), 2.67 (s, 4H), 1.35 (d, | |
| | | J = 6.6 Hz, 3H). | |
| | | Isomer-3_D2E1: ¹H NMR | |
| | | (400 MHz, Chloroform-d): δ | |
| | | 11.34 (s, 1H), 9.69 (s, 1H), | |
| | | 9.15 (s, 1H), 8.74 (s, 1H), | |
| | | 7.75 (dd, J = 8.0, 1.3 Hz, | |
| | | 1H), 7.69 − 7.64 (m, 1H), | |
| | | 7.50 (d, J = 7.9 Hz, 1H), 7.47 − | |
| | | 7.41 (m, 1H), 7.32 (d, J = | |
| | | 0.9 Hz, 1H), 7.18 (s, 1H), | |
| | | 5.45 (d, J = 10.2 Hz, 1H), | |
| | | 4.15-4.08 (m, 2H), 3.68-3.54 | |
| | | (m, 8H), 2.71 (s, 2H), 2.39 | |
| | | (s, 4H), 1.05 (d, J = 6.9 Hz, | |
| | | 3H). | |
| | | Isomer-4_D2E1: ¹H NMR | |
| | | (400 MHz, Chloroform-d): δ | |
| | | 11.33 (s, 1H), 9.68 (s, 1H), | |
| | | 9.15 (s, 1H), 8.74 (s, 1H), | |
| | | 7.75 (dd, J = 7.9, 1.4 Hz, | |
| | | 1H), 7.69 − 7.64 (m, 1H), | |
| | | 7.51 (d, J = 7.9 Hz, 1H), 7.46 − | |
| | | 7.41 (m, 1H), 7.31 (s, 1H), | |
| | | 7.23 (s, 1H), 5.45 (d, J = 10.2 | |
| | | Hz, 1H), 4.15-4.08 (m, 2H), | |
| | | 3.68 − 3.55 (m, 8H), 2.72 (s, | |
| | | 2H), 2.41 (s, 4H), 1.05 (d, J = | |
| | | 6.8 Hz, 3H). | |
| Example 21 | 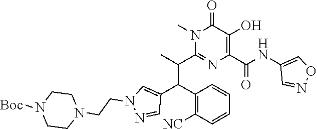 [SMILES: CC(c1nc(C(=O)Nc2cnoc2)c(O)c(=O)n1C)C(c1cnn(CCN2CCN(C(=O)OC(C)(C)C)CC2)c1)c1ccccc1C#N] | Isomer-1_D1E1: ¹H NMR (400 MHz, DMSO-d₆): δ 11.15 (s, 1H), 10.24 (s, 1H), 9.29 (s, 1H) 8.84 (s, 1H) 7.83-7.75 (m, 2H), 7.60 − 7.45 (m, 3H), 7.22-7.13 (m, 1H), 5.00-4.88 (m, 1H), 4.17 (d, J = 6.7 Hz, 2H), 4.08-3.89 | Isomer-1_D1E1: m/ [symbol] 658.4 [M + H] Isomer-2_D1E2: m/ [symbol] 658.4 [M + H] Isomer-3_D2E1: m/ [symbol] 658.4 [M + H] Isomer-4_D2E2: m/ [symbol] 658.4 [M + H] |
| | | (m, 1H), 3.59 (d, J = 11.0 Hz, | |
| | | 3H), 3.21 (t, J = 5.0 Hz, 4H), | |
| | | 2.70 − 2.64 (m, 3H), 2.34 − | |
| | | 2.27 (m, 4H), 1.38 (s, 9H), | |
| | | 1.34 − 1.21 (m, 3H) | |
| | | Isomer-2_D1E2: ¹H NMR | |
| | | (400 MHz, DMSO-d₆): δ | |
| | | 11.20 (s, 1H), 10.53 (s, 1H), | |
| | | 9.29 (s, 1H), 8.86 (s, 1H), | |
| | | 7.88 − 7.73 (m, 2H), 7.63 − | |
| | | 7.46 (m, 3H), 7.20 (m, 1H), | |
| | | 4.98 (d, J = 11.0 Hz, 1H), | |
| | | 4.25 − 3.99 (m, 3H), 3.60 (s, | |
| | | 3H), 3.21 (t, J = 5.0 Hz, 4H), | |
| | | 2.73 − 2.63 (m, 3H), 2.36 − | |
| | | 2.21 (m, 4H), 1.38 (s, 9H), | |
| | | 1.32 (d, J = 6.5 Hz, 3H). | |
| | | Isomer-3_D2E1: ¹H NMR | |
| | | (300 MHz, DMSO-d₆): δ | |
| | | 11.28 (s, 1H) 9.31(s, 1H) | |
| | | 8.87(s, 1H) 7.97 (d, J = 7.9 | |
| | | Hz, 2H), 7.87 − 7.73 (m, | |
| | | 2H), 7.52 − 7.36 (m, 1H), | |
| | | 7.05 (s, 1H), 4.82 (d, J = 10.5 | |
| | | Hz, 1H), 4.01 (d, J = 6.5 Hz, | |
| | | 3H), 3.44 (s, 3H), 3.16 (s, | |
| | | 4H), 2.20 (s, 4H), 1.39 (s, | |
| | | 9H), 1.17 (d, J = 6.4 Hz, 3H) | |
| | | Isomer-4_D2E1: ¹H NMR | |
| | | (300 MHz, DMSO-d₆): δ | |
| | | 11.25 (s, 1H) 9.31 (s, 1H), | |
| | | 8.88 (s, 1H), 7.97 (d, J = 8.0 | |
| | | Hz, 1H), 7.87 − 7.71 (m, | |
| | | 2H), 7.51 − 7.33 (m, 2H), | |
| | | 7.05 (s, 1H) 4.82 (d, J = | |
| | | 10.5 Hz, 1H), 4.01 (d, J = 6.8 | |
| | | Hz, 3H), 3.45 (s, 3H), 3.16 | |
| | | (s, 4H), 2.20 (s, 4H), 1.38 (d, | |
| | | J = 5.3 Hz, 9H), 1.17 (d, J = | |
| | | 6.3 Hz, 3H) | |
| Example 22 | 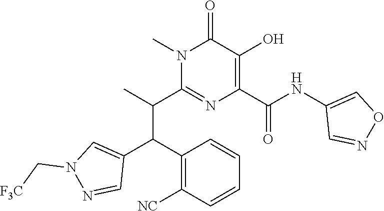 [SMILES: CC(c1nc(C(=O)Nc2cnoc2)c(O)c(=O)n1C)C(c1cnn(CC(F)(F)F)c1)c1ccccc1C#N] | Isomer-1_D1E1: ¹H NMR (400 MHz, DMSO-d₆): δ 11.17 (s, 1H), 10.46 (s, 1H), 9.30 (s, 1H), 8.87 (s, 1H), 7.95 (s, 1H), 7.86 − 7.84(m, 1H), 7.80 (s, 1H), 7.67 − 7.48 (m, 2H), 7.26 − 7.20 (m, 1H), 5.20 − 4.98 (m, 3H), 4.22 − 4.02 (m, 1H), 3.61 (s, 3H), 1.32 (d, 3H). Isomer-2_D1E2: ¹H NMR (400 MHz, DMSO-d₆): δ 11.17 (s, 1H), 10.46 (s, 1H), 9.30 (s, 1H), 8.87 (s, 1H), | |
| | | 7.95 (s, 1H), 7.86 − 7.84(m, | |
| | | 1H), 7.80 (s, 1H), 7.67 − 7.48 | |
| | | (m, 2H), 7.26 − 7.20 (m, 1H), | |
| | | 5.20 − 4.98 (m, 3H), 4.22 − | |
| | | 4.02 (m, 1H), 3.61 (s, 3H), | |
| | | 1.32 (d, 3H). | |
| | | Isomer-3_D2E1: ¹H NMR | |
| | | (400 MHz, DMSO-d₆): δ | |
| | | 11.28 (s, 1H), 10.43 (s, 1H), | |
| | | 9.34 (s, 1H), 8.90 (s, 1H), | |
| | | 8.03 − 8.01 (m, 1H), 7.91 − | |
| | | 7.75 (m, 2H), 7.57 − 7.43 (m, | |
| | | 2H), 7.27 (s, 1H), 4.98 − 4.87 | |
| | | (m, 3H), 4.12 − 4.04 (m, 1H), | |
| | | 3.46 (s, 3H), 1.20 (d, 3H). | |
| | | Isomer-4_D2E2: ¹H NMR | |
| | | (400 MHz, DMSO-d₆): δ | |
| | | 11.28 (s, 1H), 10.43 (s, 1H), | |
| | | 9.34 (s, 1H), 8.90 (s, 1H), | |
| | | 8.03 − 8.01 (m, 1H), 7.91 − | |
| | | 7.75 (m, 2H), 7.57 − 7.43 (m, | |
| | | 2H), 7.27 (s, 1H), 4.98 − 4.87 | |
| | | (m, 3H), 4.10 − 4.06 (m, 1H), | |
| | | 3.46 (s, 3H), 1.20 (d, 3H). | |
| Example 23 | 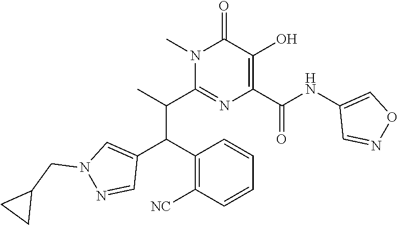 [SMILES: CC(c1nc(C(=O)Nc2cnoc2)c(O)c(=O)n1C)C(c1cnn(CC2CC2)c1)c1ccccc1C#N] | Isomer-1_D1E1: ¹H NMR (400 MHz, DMSO-d₆): δ 11.18 (s, 1H), 10.43 (s, 1H), 9.30 (s, 1H), 8.88 (s, 1H), 7.85 − 7.81 (m, 2H), 7.61 − 7.55 (m, 3H), 7.23-7.19 (m, 1H), 5.00 (d, 1H), 4.11 − 3.93 (m, 1H), 4.01 − 3.87 (m, 2H), 3.60 (s, 3H), 1.34 (d, 3H), 1.21 − 1.18 (m, 1H), 0.55 − 0.47 (m, 2H), 0.37 − 0.29 (m, 2H). Isomer-2_D1E2: ¹H NMR (400 MHz, DMSO-d₆): δ 11.18 (s, 1H), 10.43 (s, 1H), | |
| | | 9.30 (s, 1H), 8.88 (s, 1H), | |
| | | 7.85 − 7.81 (m, 2H), 7.61 − | |
| | | 7.54 (m, 3H), 7.23-7.19 (m, | |
| | | 1H), 5.00 (d, 1H), 4.11 − | |
| | | 4.07 (m, 1H), 3.95 − 3.93 (m, | |
| | | 2H), 3.60 (s, 3H), 1.34 (d, | |
| | | 3H), 1.22 − 1.18 (m, 1H), | |
| | | 0.53 − 0.52 (m, 2H), 0.37 − | |
| | | 0.29 (m, 2H). | |
| | | Isomer-3_D2E1: ¹H NMR | |
| | | (400 MHz, DMSO-d₆): δ | |
| | | 11.27 (s, 1H), 10.43 (s, 1H), | |
| | | 9.34 (s, 1H), 8.90 (s, 1H), | |
| | | 8.00 (d, 1H), 7.85 − 7.79 (m, | |
| | | 2H), 7.51-7.47 (m, 1H), 7.38 | |
| | | (s, 1H), 7.09 (s, 1H), 4.81 (d, | |
| | | 1H), 4.08 − 4.04 (m, 1H), | |
| | | 3.79 − 3.76 (m, 2H), 3.44 (s, | |
| | | 3H), 1.20 (d, 3H), 1.01-0.97 | |
| | | (m, 1H), 0.38 − 0.35 (m, 2H), | |
| | | 0.15 − 0.14 (m, 2H). | |
| | | Isomer-4_D2E2: ¹H NMR | |
| | | (400 MHz, DMSO-d₆): δ | |
| | | 11.27 (s, 1H), 10.43 (s, 1H), | |
| | | 9.34 (s, 1H), 8.90 (s, 1H), | |
| | | 8.00 (d, 1H), 7.85 − 7.79 (m, | |
| | | 2H), 7.51-7.47 (m, 1H), 7.38 | |
| | | (s, 1H), 7.09 (s, 1H), 4.81 (d, | |
| | | 1H), 4.08 − 4.04 (m, 1H), | |
| | | 3.79 − 3.76 (m, 2H), 3.44 (s, | |
| | | 3H), 1.20 (d, 3H), 1.01-0.97 | |
| | | (m, 1H), 0.38 − 0.35 | |
| Example 24 | 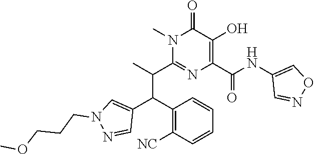 [SMILES: COCCCn1cc(C(c2ccccc2C#N)C(C)c2nc(C(=O)Nc3cnoc3)c(O)c(=O)n2C)cn1] | Isomer-1_D1E1: ¹H NMR (400 MHz, DMSO-d₆): δ 11.18 (s, 1H), 10.45 (s, 1H), 9.29 (s, 1H), 8.87 (s, 1H), 7.82-7.80 (m, 2H), 7.60 − 7.56 (m, 3H), 7.22-7.21 (m, 1H), 4.99 (d, 1H), 4.18-7.00 (m, 3H), 3.60 (s, 3H), 3.21 − 3.19 (m, 5H), 1.99-1.95 (m, 2H), 1.33 (d, 3H). | |
| | | Isomer-2_D1E2: ¹H NMR | |
| | | (400 MHz, DMSO-d₆): δ | |
| | | 11.18 (s, 1H), 10.45 (s, 1H), | |
| | | 9.29 (s, 1H), 8.87 (s, 1H), | |
| | | 7.82-7.80 (m, 2H), 7.60 − | |
| | | 7.56 (m, 3H), 7.22-7.21 (m, | |
| | | 1H), 4.99 (d, 1H), 4.18-7.00 | |
| | | (m, 3H), 3.60 (s, 3H), 3.21 − | |
| | | 3.19 (m, 5H), 1.99-1.95 (m, | |
| | | 2H), 1.33 (d, 3H). | |
| | | Isomer-2_D1E2: ¹H NMR | |
| | | (400 MHz, DMSO-d₆): δ | |
| | | 11.18 (s, 1H), 10.47 (s, 1H), | |
| | | 9.29 (s, 1H), 8.87 (s, 1H), | |
| | | 7.82-7.79 (m, 2H), 7.60 − | |
| | | 7.54 (m, 3H), 7.21 (t, 1H), | |
| | | 4.99 (d, 1H), 4.11-4.10 (m, | |
| | | 3H), 3.60 (s, 3H), 3.20 (d, | |
| | | 5H), 1.96 (p, 2H), 1.33 (d, | |
| | | 3H). | |
| | | Isomer-3_D2E1: ¹H NMR | |
| | | (400 MHz, DMSO-d₆): δ | |
| | | 11.28 (s, 1H), 10.49 (s, 1H), | |
| | | 9.33 (s, 1H), 8.89 (s, 1H), | |
| | | 7.98 (d, 1H), 7.85 − 7.78 (m, | |
| | | 2H), 7.50 − 7.46 (m, 1H), | |
| | | 7.38 (s, 1H), 7.11 (s, 1H), | |
| | | 4.83 (d, 1H), 4.06 − 3.94 (m, | |
| | | 3H), 3.47 (s, 3H), 3.10 (s, | |
| | | 3H), 3.01 (tt, 2H), 1.78 (p, | |
| | | 2H), 1.18 (d, 3H). | |
| | | Isomer-4_D2E2: ¹H NMR | |
| | | (400 MHz, DMSO-d₆): δ | |
| | | 11.26 (s, 1H), 10.41 (s, 1H), | |
| | | 9.34 (s, 1H), 8.89 (s, 1H), | |
| | | 7.99 − 7.78 (m, 3H), 7.50 − | |
| | | 7.38 (m, 2H), 7.12 (s, 1H), | |
| | | 4.83 (d, 1H), 4.08 − 3.94 (m, | |
| | | 3H), 3.47 (s, 3H), 3.10 (s, | |
| | | 3H), 3.04 − 3.00 (m, 2H), | |
| | | 1.78 (p, 2H), 1.19 (d, 3H). | |
| Example 25 | 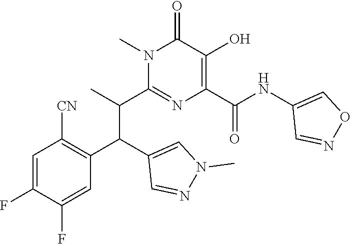 [SMILES: CC(c1nc(C(=O)Nc2cnoc2)c(O)c(=O)n1C)C(c1cnn(C)c1)c1cc(F)c(F)cc1C#N] | Isomer-1_D1E1: ¹H NMR (400 MHz, DMSO-d₆) δ 11.18 (s, 1H), 10.41 (s, 1H), 9.29 (s, 1H), 8.84 (s, 1H), 8.01 (dd, 1H), 7.91 (dd, 1H), 7.79 (s, 1H), 7.61 (s, 1H), 5.03 (d, 1H), 4.12 − 3.95 (m, 1H), 3.81 (s, 3H), 3.63 (s, 3H), 1.29 (d, 3H). Isomer-2_D1E2: ¹H NMR (400 MHz, DMSO-d₆) δ 11.18 (s, 1H), 10.41 (s, 1H), 9.29 (s, 1H), 8.84 (s, 1H), 8.01 (dd, 1H), 7.91 (dd, 1H), 7.79 (s, 1H), 7.61 (s, 1H), | Isomer-1_D1E1: m/ [symbol] 496.2 [M + H] >98% ee Isomer-2_D1E2: m/ [symbol] 496.2 [M + H] >98% ee Isomer-3_D2E1: m/ [symbol] 496.2 [M + H] >98% ee Isomer-4_D2E2: m/ [symbol] 496.2 [M + H] >98% ee |
| | | 5.03 (d, 1H), 4.12 − 3.95 (m, | |
| | | 1H), 3.81 (s, 3H), 3.63 (s, | |
| | | 3H), 1.29 (d, 3H). | |
| | | Isomer-3_D2E1: ¹H NMR | |
| | | (400 MHz, DMSO-d₆) δ | |
| | | 11.19 (s, 1H), 10.35 (s, 1H), | |
| | | 9.32 (s, 1H), 8.85 (s, 1H), | |
| | | 8.18 − 8.10 (m, 2H), 7.43 (s, | |
| | | 1H), 7.15 (d, 1H), 4.86 (d, | |
| | | 1H), 4.12 − 3.95 (m, 1H), | |
| | | 3.66 (s, 3H), 3.49 (s, 3H), | |
| | | 1.18 (d, 3H). | |
| | | Isomer-4_D2E2: ¹H NMR | |
| | | (400 MHz, DMSO-d₆) δ | |
| | | 11.19 (s, 1H), 10.35 (s, 1H), | |
| | | 9.32 (s, 1H), 8.85 (s, 1H), | |
| | | 8.18 − 8.10 (m, 2H), 7.43 (s, | |
| | | 1H), 7.15 (d, 1H), 4.86 (d, | |
| | | 1H), 4.12 − 3.95 (m, 1H), | |
| | | 3.66 (s, 3H), 3.49 (s, 3H), | |
| | | 1.18 (d, 3H). | |
| Example 26 | 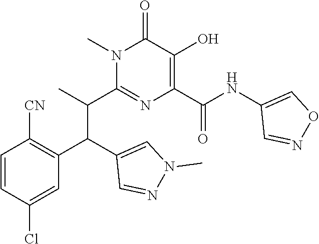 [SMILES: CC(c1nc(C(=O)Nc2cnoc2)c(O)c(=O)n1C)C(c1cnn(C)c1)c1cc(Cl)ccc1C#N] | Isomer-1_D1E1: ¹H NMR (400 MHz, DMSO-d₆) δ 11.23 (s, 1H), 10.49 (s, 1H), 9.29 (s, 1H), 8.86 (s, 1H), 7.96 (d, 1H), 7.83 (s, 1H), 7.65 − 7.63 (m, 2H), 7.31- 7.29 (m, 1H), 5.00 (d, 1H), 4.15-4.11 (m, 1H), 3.82 (s, 3H), 3.62 (s, 3H), 1.32 (d, 3H). Isomer-2_D1E2: ¹H NMR (400 MHz, DMSO-d₆) δ 11.20 (s, 1H), 10.45 (s, 1H), 9.29 (s, 1H), 8.86 (s, 1H), 7.96 (d, 1H), 7.83 (s, 1H), | Isomer-1_D1E1: m/ [symbol] 494.2 [M + H] >98% ee Isomer-2_D1E2: m/ [symbol] 494.2 [M + H] >98% ee Isomer-3_D2E1: m/ [symbol] 494.4 [M + H] >98% ee Isomer-4_D2E2: m/ [symbol] 494.2 [M + H] >98% ee |
| | | 7.65 − 7.63 (m, 2H), 7.31- | |
| | | 7.29 (m, 1H), 5.00 (d, 1H), | |
| | | 4.16-4.12 (m, 1H), 3.82 (s, | |
| | | 3H), 3.62 (s, 3H), 1.32 (d, | |
| | | 3H). | |
| | | Isomer-3_D2E1: ¹H NMR | |
| | | (400 MHz, DMSO-d₆) δ | |
| | | 11.17 (s, 1H), 10.35 (s, 1H), | |
| | | 9.33 (s, 1H), 8.86 (s, 1H), | |
| | | 8.08 (d, 1H), 7.89 (d, 1H), | |
| | | 7.58-7.56 (m, 1H), 7.43 (s, | |
| | | 1H), 7.16 (s, 1H), 4.91 (d, | |
| | | 1H), 4.13-4.09 (m, 1H), 3.66 | |
| | | (s, 3H), 3.52 (s, 3H), 1.17 (d, | |
| | | 3H). | |
| | | Isomer-4_D2E2: ¹H NMR | |
| | | (400 MHz, DMSO-d₆) δ | |
| | | 11.17 (s, 1H), 10.35 (s, 1H), | |
| | | 9.33 (s, 1H), 8.86 (s, 1H), | |
| | | 8.08 (d, 1H), 7.89 (d, 1H), | |
| | | 7.58-7.56 (m, 1H), 7.43 (s, | |
| | | 1H), 7.16 (s, 1H), 4.91 (d, | |
| | | 1H), 4.13-4.09 (m, 1H), 3.66 | |
| | | (s, 3H), 3.52 (s, 3H), 1.17 (d, | |
| | | 3H). | |
| Example 27 | 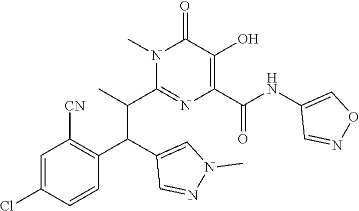 [SMILES: CC(c1nc(C(=O)Nc2cnoc2)c(O)c(=O)n1C)C(c1cnn(C)c1)c1ccc(Cl)cc1C#N] | Isomer-1_D1E1: ¹H NMR (400 MHz, DMSO-d₆) δ 11.19 (s, 1H), 10.50 (s, 1H), 9.29 (s, 1H), 8.85 (s, 1H), 7.86 − 7.70 (m, 3H), 7.63 (dd, 1H), 7.58 (s, 1H), 5.00 (d, 1H), 4.06 (dt, 1H), 3.80 (s, 3H), 3.61 (s, 3H), 1.32 (d, 3H). Isomer-2_D1E2: ¹H NMR (400 MHz, DMSO-d₆) δ 11.18 (s, 1H), 10.47 (s, 1H), 9.29 (s, 1H), 8.85 (s, 1H), | Isomer-1_D1E1: m/ [symbol] 494.2 [M + H] >98% ee Isomer-2_D1E2: m/ [symbol] 494.2 [M + H] >98% ee Isomer-3_D2E1: m/ [symbol] 494.2 [M + H] >98% ee Isomer-4_D2E2: m/ [symbol] 494.2 [M + H] >98% ee |
| | | 7.86 − 7.76 (m, 3H), 7.63 | |
| | | (dd, 1H), 7.58 (d, 1H), 5.00 | |
| | | (d, 1H), 4.07 (dq, 1H), 3.80 | |
| | | (s, 3H), 3.61 (s, 3H), 1.32 (d, | |
| | | 3H). | |
| | | Isomer-3_D2E1: ¹H NMR | |
| | | (400 MHz, DMSO-d₆) δ | |
| | | 11.22 (s, 1H), 10.39 (s, 1H), | |
| | | 9.33 (s, 1H), 8.86 (s, 1H), | |
| | | 8.03 (d, 1H), 7.96 (d, 1H), | |
| | | 7.87 (dd, 1H), 7.40 (s, 1H), | |
| | | 7.12 (s, 1H), 4.87 (d, 1H), | |
| | | 4.04 (dd, 1H), 3.65 (s, 3H), | |
| | | 3.49 (s, 3H), 1.16 (d, 3H). | |
| | | Isomer-4_D2E2: ¹H NMR | |
| | | (400 MHz, DMSO-d₆) δ | |
| | | 11.22 (s, 1H), 10.36 (s, 1H), | |
| | | 9.33 (s, 1H), 8.86 (s, 1H), | |
| | | 8.03 (d, 1H), 7.98 − 7.88 (m, | |
| | | 1H), 7.88 − 7.79 (m, 1H), | |
| | | 7.40 (s, 1H), 7.12 (s, 1H), | |
| | | 4.87 (d, 1H), 4.04 (dt, 1H), | |
| | | 3.66 (s, 3H), 3.50 (s, 3H), | |
| | | 1.16 (d, 3H). | |
| Example 28 | 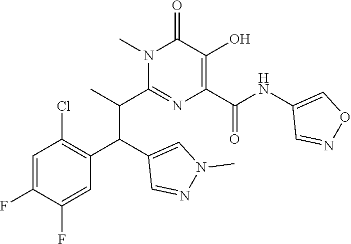 [SMILES: CC(c1nc(C(=O)Nc2cnoc2)c(O)c(=O)n1C)C(c1cnn(C)c1)c1cc(F)c(F)cc1Cl] | Isomer-1_D1E1: ¹H NMR (400 MHz, DMSO-d₆) δ 11.14 (s, 1H), 10.38 (s, 1H), 9.33 (s, 1H), 8.94 (s, 1H), 7.84 (dd, J = 12.0, 8.5 Hz, 1H), 7.74 (s, 1H), 7.57 (s, 1H), 7.48 (dd, J = 10.4, 7.6 Hz, 1H), 5.10 (d,J = 11.1 Hz, 1H), 4.02 (dq, J = 13.6, 6.9 Hz, 1H), 3.80 (s, 3H), 3.65 (s, 3H), 1.27 (d, J = 6.6 Hz, 3H). Isomer-2_D1E2: ¹H NMR (400 MHz, DMSO-d₆) δ 11.15 (s, 1H), 10.40 (s, 1H), | Isomer-1_D1E1: m/ [symbol] 505.2 [M + H] >98% ee Isomer-2_D1E2: m/ [symbol] 505.2 [M + H] >98% ee Isomer-3_D2E1: m/ [symbol] 505.2 [M + H] >98% ee Isomer-4_D2E2: m/ [symbol] 505.2 [M + H] >98% ee |
| | | 9.33 (s, 1H), 8.94 (s, 1H), | |
| | | 7.84 (dd, J = 12.0, 8.6 Hz, | |
| | | 1H), 7.74 (s, 1H), 7.57 (s, | |
| | | 1H), 7.48 (dd, J = 10.4, 7.6 | |
| | | Hz, 1H), 5.19 −5.03 (m, 1H), | |
| | | 4.02 (dq, J = 13.4, 6.8 Hz, | |
| | | 1H), 3.80 (s, 3H), 3.65 (s, | |
| | | 3H), 1.27 (d, J = 6.5 Hz, 3H). | |
| | | Isomer-3_D2E1: ¹H NMR | |
| | | (400 MHz, DMSO-d₆) δ | |
| | | 11.36 (s, 1H), 9.34 (s, 1H), | |
| | | 8.93 (s, 1H), 8.07 (t, J = 10.3 | |
| | | Hz, 1H), 7.75 − 7.65 (m, | |
| | | 1H), 7.32 (s, 1H), 7.02 (s, | |
| | | 1H), 4.84 (d, J = 10.8 Hz, | |
| | | 1H), 3.97 (s, 1H),3.55 (d, J = | |
| | | 66.1 Hz, 6H), 1.20 (d, J = 6.5 | |
| | | Hz, 3H). | |
| | | Isomer-4_D2E2: ¹H NMR | |
| | | (400 MHz, DMSO-d₆) δ | |
| | | 11.37 (s, 1H), 10.56 (s, 1H), | |
| | | 9.34 (s, 1H), 8.93 (s, 1H), | |
| | | 8.09 (q, J = 11.8, 10.1 Hz, | |
| | | 1H), 7.70 (dd, J = 10.2, 7.9 | |
| | | Hz, 1H), 7.32 (s, 1H), 7.02 | |
| | | (s, 1H), 4.84 (d, J= 10.6 Hz, | |
| | | 1H), 3.97 (s, 1H), 3.63 (s, | |
| | | 3H), 3.46 (s, 3H), 1.20 (d, J = | |
| | | 6.5 Hz, 3H). | |
| Example 29 | 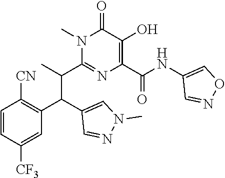 [SMILES: CC(c1nc(C(=O)Nc2cnoc2)c(O)c(=O)n1C)C(c1cnn(C)c1)c1cc(C(F)(F)F)ccc1C#N] | | Isomer-1_D1E1: m/ [symbol] 528.0 [M + H] >98% ee Isomer-2_D1E2: m/ [symbol] 528.0 [M + H] >98% ee Isomer-3_D2E1: m/ [symbol] 528.0 [M + H] >98% ee Isomer-4_D2E2: m/ [symbol] 528.0 [M + H] >98% ee |
| Example 30 | 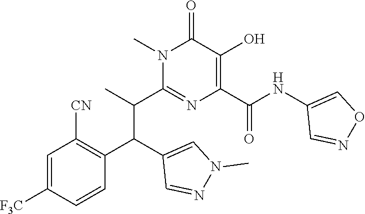 [SMILES: CC(c1nc(C(=O)Nc2cnoc2)c(O)c(=O)n1C)C(c1cnn(C)c1)c1ccc(C(F)(F)F)cc1C#N] | Isomer-1_D1E1: ¹H NMR (300 MHz, DMSO-d₆): δ 11.15 (s, 1H), 10.44 (s, 1H), 9.30 (s, 1H), 8.86 (s, 1H), 8.13 (d, 1H), 8.05 (d, 1H), 7.92 (dd, 1H), 7.81 (s, 1H), 7.62 (s, 1H), 5.14 (d, 1H), 4.20 − 4.14 (m, 1H), 3.81 (s, 3H), 3.64 (s, 3H), 1.34 (d, 3H). Isomer-2_D1E2: ¹H NMR (300 MHz, DMSO-d₆): δ 11.15 (s, 1H), 10.44 (s, 1H), | Isomer-1_D1E1: m/ [symbol] 528.2 [M + H] >98% ee Isomer-2_D1E2: m/ [symbol] 528.2 [M + H] >98% ee Isomer-3_D2E1: m/ [symbol] 528.2 [M + H] >98% ee Isomer-4_D2E2: m/ [symbol] 528.2 [M + H] >98% ee |
| | | 9.30 (s, 1H), 8.86 (s, 1H), | |
| | | 8.13 (d, 1H), 8.05 (d, 1H), | |
| | | 7.92 (dd, 1H), 7.81 (s, 1H), | |
| | | 7.62 (s, 1H), 5.14 (d, 1H), | |
| | | 4.23 − 4.12 (m, 1H), 3.81 (s, | |
| | | 3H), 3.64 (s, 3H), 1.34 (d, | |
| | | 3H). | |
| | | Isomer-3_D2E1: ¹H NMR | |
| | | (300 MHz, DMSO-d₆): δ | |
| | | 11.23 (s, 1H), 10.34 (s, 1H), | |
| | | 9.33 (s, 1H), 8.86 (s, 1H), | |
| | | 8.34 (d, 1H), 8.25 − 8.07 (m, | |
| | | 2H), 7.45 (s, 1H), 7.18 (d, | |
| | | 1H), 5.00 (d, 1H), 4.16 − | |
| | | 4.10 (m, 1H), 3.67 (s, 3H), | |
| | | 3.53 (s, 3H), 1.18 (d, 3H). | |
| | | Isomer-4_D2E2: ¹H NMR | |
| | | (300 MHz, DMSO-d₆): δ | |
| | | 11.23 (s, 1H), 10.36 (s, 1H), | |
| | | 9.33 (s, 1H), 8.86 (s, 1H), | |
| | | 8.34 (d, 1H), 8.25 − 8.07 (m, | |
| | | 2H), 7.45 (s, 1H), 7.18 (d, | |
| | | 1H), 5.00 (d, 1H), 4.16 − | |
| | | 4.10 (m, 1H), 3.67 (s, 3H), | |
| | | 3.53 (s, 3H), 1.18 (d, 3H). | |
| Example 58 | 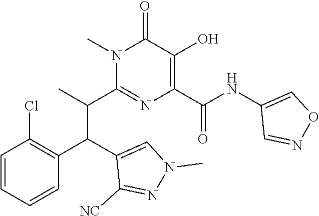 [SMILES: CC(c1nc(C(=O)Nc2cnoc2)c(O)c(=O)n1C)C(c1ccccc1Cl)c1cn(C)nc1C#N] | | Isomer-1_D1E1: m/ [symbol] 494.1 [M + H] >98% ee Isomer-2_D1E2: m/ [symbol] 494.1 [M + H] >98% ee Isomer-3_D2E1: m/ [symbol] 494.1 [M + H] >98% ee Isomer-4_D2E2: m/ [symbol] 494.1 [M + H] >98% ee |
| Example 59 | 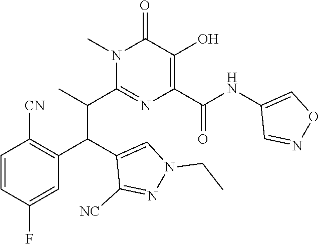 | | Isomer-1_D1E1: m/ [symbol] 503.2 [M + H] >98% ee Isomer-2_D1E2: m/ [symbol] 503.2 [M + H] >98% ee |
| Example 60 | 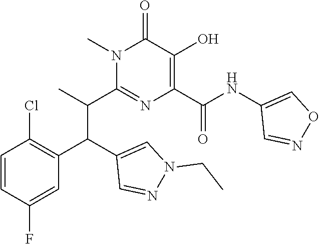 [SMILES: CCn1cc(C(c2cc(F)ccc2Cl)C(C)c2nc(C(=O)Nc3cnoc3)c(O)c(=O)n2C)cn1] | Isomer-1_D1E1 ¹H NMR (300 MHz, DMSO- d₆) δ 11.14 (s, 1H), 10.35 (s, 1H), 9.33 (s, 1H), 8.95 (s, 1H), 7.81 (s, 1H), 7.62 − 7.58 (m, 2H), 7.27 (dd, 1H), 6.89 (td, 1H), 5.12 (d, 1H), 4.14- 4.02 (m, 3H), 3.65 (s, 3H), 1.36 (t, 3H), 1.26 (d, 3H). Isomer-2_D1E2 ¹H NMR (300 MHz, DMSO- d₆) δ 11.14 (s, 1H), 10.34 (s, 1H), 9.33 (s, 1H), 8.95 (s, 1H), 7.81 (s, 1H), 7.62 − 7.58 (m, 2H), 7.27 (dd, 1H), 6.89 | Isomer-1_D1E1: m/ [symbol] 501.2 [M + H] >98% ee Isomer-2_D1E2: m/ [symbol] 501.2 [M + H] >98% ee Isomer-3_D2E1 m/ [symbol] 501.3 [M + H] >98% ee Isomer-4_D2E2 m/ [symbol] 501.2 [M + H] >98% ee |
| | | (td, 1H), 5.14 − 5.10 (m, 1H), | |
| | | 4.14 − 4.02 (m, 3H), 3.65 (s, | |
| | | 3H), 1.36 (t, 3H), 1.26 (d, | |
| | | 3H). | |
| | | Isomer-3_D2E1 | |
| | | ¹H NMR (400 MHz, DMSO- | |
| | | d₆) δ 11.41 (s, 1H), 10.53 (s, | |
| | | 1H), 9.35 (s, 1H), 8.95 (s, | |
| | | 1H), 7.86 (d, 1H), 7.50 (dd, | |
| | | 1H), 7.32 (s, 1H), 7.17 (td, | |
| | | 1H), 7.01 (s, 1H), 4.80 (d, | |
| | | 1H), 3.98-3.89 (m, 3H), | |
| | | 3.41(s, 3H), 1.23 (d, 3H), | |
| | | 1.14 (t, 3H). | |
| | | Isomer-4_D2E2 | |
| | | ¹H NMR (400 MHz, DMSO- | |
| | | d₆) δ 11.39 (s, 1H), 10.51 (s, | |
| | | 1H), 9.34 (s, 1H), 8.95 (s, | |
| | | 1H), 7.85 (d, 1H), 7.49 (dd, | |
| | | 1H), 7.32 (s, 1H), 7.17 (td, | |
| | | 1H), 7.01 (s, 1H), 4.80 (d, | |
| | | 1H), 3.98-3.89 (m, 3H), 3.42 | |
| | | (s, 3H), 1.23 (d, 3H), 1.15 (t, | |
| | | 3H). | |
| Example 68 | 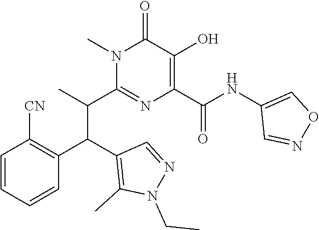 [SMILES: CCn1ncc(C(c2ccccc2C#N)C(C)c2nc(C(=O)Nc3cnoc3)c(O)c(=O)n2C)c1C] | | Isomer-1_D1E1: m/ [symbol] 488.0 [M + H] >98% ee Isomer-2_D1E2: m/ [symbol] 488.0 [M + H] >98% ee Isomer-3_D2E1: m/ [symbol] 488.0 [M + H] >98% ee Isomer-4_D2E2: m/ [symbol] 488.0 [M + H] >98% ee |
| Example 69 | 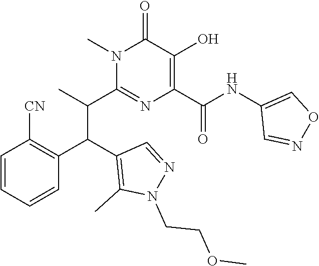 [SMILES: COCCn1ncc(C(c2ccccc2C#N)C(C)c2nc(C(=O)Nc3cnoc3)c(O)c(=O)n2C)c1C] | | Isomer-1_D1E1: m/ [symbol] 518.0 [M + H] >98% ee Isomer-2_D1E2: m/ [symbol] 518.0 [M + H] >98% ee Isomer-3_D2E1: m/ [symbol] 518.0 [M + H] >98% ee Isomer-4_D2E2: m/ [symbol] 518.0 [M + H] >98% ee |
| Example 70 | 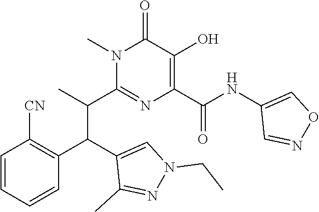 [SMILES: CCn1cc(C(c2ccccc2C#N)C(C)c2nc(C(=O)Nc3cnoc3)c(O)c(=O)n2C)c(C)n1] | | Isomer-1_D1E1: m/ [symbol] 488.3 [M + H] >98% ee Isomer-2_D1E2: m/ [symbol] 488.3 [M + H] >98% ee Isomer-3_D2E1: m/ [symbol] 488.3 [M + H] >98% ee Isomer-4_D2E2: m/ [symbol] 488.3 [M + H] >98% ee |
| Example 71 | 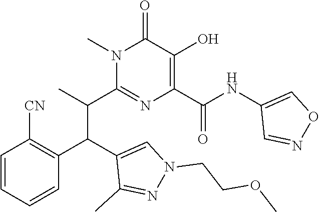 [SMILES: COCCn1cc(C(c2ccccc2C#N)C(C)c2nc(C(=O)Nc3cnoc3)c(O)c(=O)n2C)c(C)n1] | | Isomer-1_D1E1: m/ [symbol] 518.0 [M + H] >98% ee Isomer-2_D1E2: m/ [symbol] 518.0 [M + H] >98% ee Isomer-3_D2E1: m/ [symbol] 518.0 [M + H] >98% ee Isomer-4_D2E2: m/ [symbol] 518.0 [M + H] >98% ee |
| Example 72 | 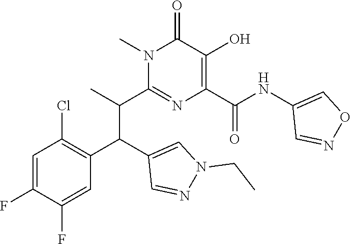 [SMILES: CCn1cc(C(c2cc(F)c(F)cc2Cl)C(C)c2nc(C(=O)Nc3cnoc3)c(O)c(=O)n2C)cn1] | | Isomer-1_D1E1: m/ [symbol] 518.9 [M + H] >98% ee Isomer-2_D1E2: m/ [symbol] 518.9 [M + H] >98% ee Isomer-3_D2E1: m/ [symbol] 518.9 [M + H] >98% ee Isomer-4_D2E2: m/ [symbol] 518.9 [M + H] >98% ee |
| Example 73 | 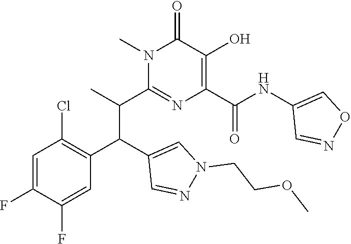 [SMILES: COCCn1cc(C(c2cc(F)c(F)cc2Cl)C(C)c2nc(C(=O)Nc3cnoc3)c(O)c(=O)n2C)cn1] | | Isomer-1_D1E1: m/ [symbol] 548.9 [M + H] >98% ee Isomer-2_D1E2: m/ [symbol] 548.9 [M + H] >98% ee Isomer-3_D2E1: m/ [symbol] 548.9 [M + H] >98% ee Isomer-4_D2E2: m/ [symbol] 548.9 [M + H] >98% ee |
| Example 74 | 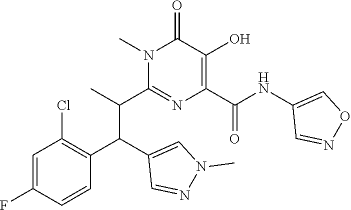 [SMILES: CC(c1nc(C(=O)Nc2cnoc2)c(O)c(=O)n1C)C(c1cnn(C)c1)c1ccc(F)cc1Cl] | | Isomer-1_D1E1: m/ [symbol] 486.9 [M + H] >98% ee Isomer-2_D1E2: m/ [symbol] 486.9 [M + H] >98% ee Isomer-3_D2E1: m/ [symbol] 486.9 [M + H] >98% ee Isomer-4_D2E2: m/ [symbol] 486.9 [M + H] >98% ee |
| Example 75 | 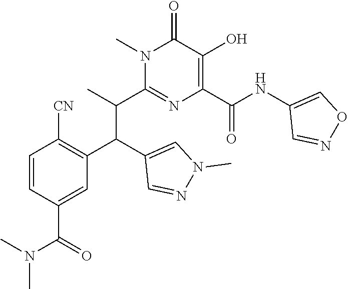 [SMILES: CC(c1nc(C(=O)Nc2cnoc2)c(O)c(=O)n1C)C(c1cnn(C)c1)c1cc(C(=O)N(C)C)ccc1C#N] | Isomer-1_D1E1: ¹H NMR (400 MHz, DMSO-d₆): δ 11.17 (s, 1H), 10.45 (s, 1H), 9.29 (s, 1H), 8.87 (s, 1H), 7.88 − 7.78 (m, 2H), 7.71 − 7.59 (m, 2H), 7.17 (dd, J = 7.9, 1.5 Hz, 1H), 5.03 (d, J = 11.0 Hz, 1H), 4.21 − 4.06 (m, 1H), 3.81 (s, 3H), 3.62 (s, 3H), 2.94 (s, 3H), 2.60 (s, 3H), 1.34 (d, J = 6.5 Hz, 3H) Isomer-2_D1E2: ¹H NMR (400 MHz, DMSO-d₆): δ 11.17 (s, 1H), 10.45 (s, 1H), 9.29 (s, 1H), 8.87 (s, 1H), 7.88 − 7.78 (m, 2H), 7.71 − 7.59 (m, 2H), 7.17 (dd, J = 7.9, 1.5 Hz, 1H), 5.03 (d, J = | Isomer-1_D1E1: m/ [symbol] 530.2 [M + H] >98% ee Isomer-2_D1E2: m/ [symbol] 530.2 [M + H] >98% ee Isomer-3_D2E1: m/ [symbol] 530.2 [M + H] >98% ee Isomer-4_D2E2: m/ [symbol] 530.2 [M + H] >98% ee |
| | | 11.0 Hz, 1H), 4.21 − 4.06 (m, | |
| | | 1H), 3.81 (s, 3H), 3.62 (s, | |
| | | 3H), 2.94 (s, 3H), 2.60 (s, | |
| | | 3H), 1.34 (d, J = 6.5 Hz, 3H) | |
| | | Isomer-3_D2E1: ¹H NMR | |
| | | (400 MHz, DMSO-d₆): δ | |
| | | 11.17 (s, 1H), 10.40 (s, 1H), | |
| | | 9.33 (s, 1H), 8.87 (s, 1H), | |
| | | 7.96 (d, J = 1.6 Hz, 1H), 7.90 | |
| | | (d, J = 7.9 Hz, 1H), 7.46 (dd, | |
| | | J = 7.8, 1.5 Hz, 1H), 7.40 (s, | |
| | | 1H), 7.15 (s, 1H), 4.95 (d, | |
| | | J = 10.4 Hz, 1H), 4.15 − 4.05 | |
| | | (m, 1H), 3.65 (s, 3H), 3.51 | |
| | | (s, 3H), 3.04 (s, 3H), 2.88 (s, | |
| | | 3H), 1.16 (d, J = 6.6 Hz, 3H). | |
| | | Isomer-4_D2E2: ¹H NMR | |
| | | (400 MHz, DMSO-d₆): δ | |
| | | 11.17 (s, 1H), 10.40 (s, 1H), | |
| | | 9.33 (s, 1H), 8.87 (s, 1H), | |
| | | 7.96 (d, J = 1.6 Hz, 1H), 7.90 | |
| | | (d, J = 7.9 Hz, 1H), 7.46 (dd, | |
| | | J = 7.8, 1.5 Hz, 1H), 7.40 (s, | |
| | | 1H), 7.15 (s, 1H), 4.95 (d, J = | |
| | | 10.4 Hz, 1H), 4.15 − 4.05 | |
| | | (m, 1H), 3.65 (s, 3H), 3.51 | |
| | | (s, 3H), 3.04 (s, 3H), 2.88 (s, | |
| | | 3H), 1.16 (d, J = 6.6 Hz, 3H). | |
| Example 76 | 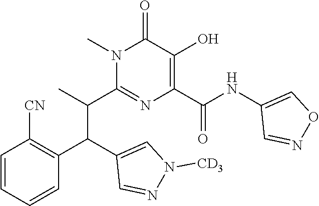 [SMILES: [2H]C([2H])([2H])n1cc(C(c2ccccc2C#N)C(C)c2nc(C(=O)Nc3cnoc3)c(O)c(=O)n2C)cn1] | | Isomer-1_D1E1: m/ [symbol] 463.2 [M + H] >98% ee Isomer-2_D1E2: m/ [symbol] 463.2 [M + H] >98% ee m/ [symbol] 463.2 [M + H] |
| Example 77 | 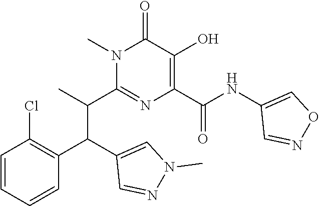 [SMILES: CC(c1nc(C(=O)Nc2cnoc2)c(O)c(=O)n1C)C(c1cnn(C)c1)c1ccccc1Cl] | | Isomer- 1_(D1E1)_LCMS: m/ [symbol] 469.6 [M⁺ + 1]. Isomer- 2_(D1E2)_LCMS m/ [symbol] 469.0 [M⁺ + 1]. Isomer- 3_(D2E1)_LCMS: m/ [symbol] 469.6 [M⁺ + 1]. Isomer- 4_(D2E2)_LCMS: m/ [symbol] 469.6 [M⁺ + 1]. |
| Example 78 | 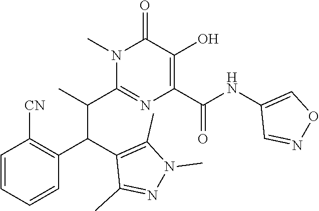 [SMILES: Cc1nn(C)c(C)c1C(c1ccccc1C#N)C(C)c1nc(C(=O)Nc2cnoc2)c(O)c(=O)n1C] | Isomer-1_ D1E1: ¹H NMR (400 MHz, DMSO-d₆): δ 1.29 (d, J = 6.4 Hz, 3H), 2.22 (s, 3H), 2.41 (s, 3H), 3.64 (s, 6H), 3.95-3.99 (m, 1H), 4.98 (d, J = 11.2 Hz, 1H), 7.22- 7.25 (m, 1H), 7.53-7.63 (m, 3H), 8.83 (s, 1H), 9.29 (s, 1H), 10.37 (s, 1H), 11.10 (s, 1H). Isomer-2_ D1E2: ¹H NMR (400 MHz, DMSO-d₆): δ 1.29 (d, J = 6.8 Hz, 3H), 2.22 (s, 3H), 2.41 (s, 3H), 3.64 (s, | Isomer- 1_(D1E1)_LCMS: m/ [symbol] 488.3 [M⁺ + 1]. Isomer- 2_(D1E2)_LCMS m/ [symbol] 488.6 [M⁺ + 1]. Isomer- 3_(D2E1)_LCMS: m/ [symbol] 487.9 [M⁺ + 1]. Isomer- 4_(D2E2)_LCMS: m/ [symbol] 488.2 [M⁺ + 1]. HPLC: FR-1 (Isomer-1; D1E1): |
| | | 6H), 3.95-3.99 (m, 1H), 4.98 | R_{T} = 4.40 (99%); |
| | | (d, J = 11.2 Hz, 1H), 7.22- | FR-2 (Isomer-2; |
| | | 7.25 (m, 1H), 7.55-7.63 (m, | D1E2): R_{T} = 4.40 |
| | | 3H), 8.83 (s, 1H), 9.29 (s, | (100%); FR-3 |
| | | 1H), 10.38 (s, 1H), 11.10 (s, | (Isomer-3; D2E1): |
| | | 1H). | R_{T} = 4.41 (99%); |
| | | Isomer-3_ D2E1: ¹H NMR | FR-4 (Isomer-4; |
| | | (400 MHz, DMSO-d₆): δ | D2E2): R_{T} = 4.42 |
| | | 1.42 (d, J = 6.4 Hz, 3H), 1.86 | (100%). |
| | | (s, 3H), 2.00 (s, 3H), 3.41 (s, | |
| | | 3H), 3.43 (s, 3H), 4.33-4.35 | |
| | | (m, 1H), 4.71 (d, J = 11.2 Hz, | |
| | | 1H), 7.46 (t, J = 7.6 Hz, 1H), | |
| | | 7.78-7.81 (m, 2H), 8.19 (d, | |
| | | J = 8.4 Hz, 1H), 8.96 (s, 1H), | |
| | | 9.36 (s, 1H), 10.81 (s, 1H), | |
| | | 11.39 (s, 1H). | |
| | | Isomer-4_ D2E2: ¹H NMR | |
| | | (400 MHz, DMSO-d₆): δ | |
| | | 1.42 (d, J = 6.0 Hz, 3H), 1.86 | |
| | | (s, 3H), 2.00 (s, 3H), 3.41 (s, | |
| | | 3H), 3.43 (s, 3H), 4.31-4.36 | |
| | | (m, 1H), 4.71 (d, J = 11.2 Hz, | |
| | | 1H), 7.46 (t, J = 7.6 Hz, 1H), | |
| | | 7.78-7.81 (m, 2H), 8.19 (d, | |
| | | J = 8.0 Hz, 1H), 8.96 (s, 1H), | |
| | | 9.36 (s, 1H), 10.80 (s, 1H), | |
| | | 11.40 (s, 1H). | |
| Example 86 | 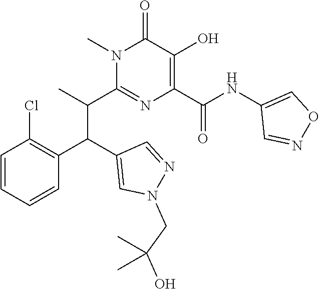 [SMILES: CC(c1nc(C(=O)Nc2cnoc2)c(O)c(=O)n1C)C(c1cnn(CC(C)(C)O)c1)c1ccccc1Cl] | Isomer-1_ D1E1: ¹H NMR (400 MHz, DMSO-d₆): δ 1.00 (s, 3H), 1.04 (s, 3H), 1.31 (t, J = 6.0 Hz, 3H), 3.62 (s, 3H), 3.96 (s, 2H), 4.03- 4.07 (m, 1H), 4.72 (bs, 1H), 5.15 (d, J = 11.2 Hz, 1H), 7.01 (t, J = 7.6 Hz, 1H), 7.17-7.21 (m, 2H), 7.55 (s, 1H), 7.66 (d, J = 7.6 Hz, 1H), 7.74 (s, 1H), 8.95 (s, 1H), 9.33 (s, 1H), 10.44 (s, 1H), 11.14 (s, 1H). Isomer-2_ D1E2: ¹H NMR (400 MHz, DMSO-d₆): δ 1.00 (s, 3H), 1.05 (s, 3H), 1.31 (t, J = 6.0 Hz, 3H), 3.62 (s, 3H), 3.97 (s, 2H), 4.03- | Isomer- 1_(D1E1)_LCMS: m/ [symbol] 527.2 [M⁺ + 1]. Isomer- 2_(D1E2)_LCMS: m/ [symbol] 527.3 [M⁺ + 1]. Isomer- 3_(D2E1)_LCMS: m/ [symbol] 527.2 [M⁺ + 1]. Isomer- 4_(D2E2)_LCMS: m/ [symbol] 527.2 [M⁺ + 1]. HPLC: FR-1 (Isomer-1; D1E1): R_{T} = 4.38 (100%); FR-2 (Isomer-2; D1E2): R_{T} = 4.39 (100%); FR-3 |
| | | 4.07 (m, 1H), 4.70 (bs, 1H), | (Isomer-3; D2E1): |
| | | 5.15 (d, J = 10.8 Hz, 1H), | R_{T} =4.53 (100%); |
| | | 7.01 (t, J = 6.8 Hz, 1H), | FR-4 (Isomer-4; |
| | | 7.17-7.21 (m, 2H), 7.56 (s, | D2E2): R_{T} = 4.53 |
| | | 1H), 7.66 (d, J = 7.6 Hz, 1H), | (100%). |
| | | 7.74 (s, 1H), 8.96 (s, 1H), | |
| | | 9.33 (s, 1H), 10.44 (s, 1H), | |
| | | 11.14 (s, 1H). | |
| | | Isomer-3_ D2E1: ¹H NMR | |
| | | (400 MHz, DMSO-d₆): δ | |
| | | 0.803 (s, 3H), 0.829 (s, 3H), | |
| | | 1.24 (bs, 3H), 3.46 (s, 3H), | |
| | | 3.78 (s, 2H), 3.95-4.02 (m, | |
| | | 1H), 4.50 (bs, 1H), 4.85 (d, | |
| | | J = 10.8 Hz, 1H), 6.99 (s, 1H), | |
| | | 7.24 (s, 1H), 7.29 (d, J = 7.2 | |
| | | Hz, 1H), 7.44-7.46 (m, 2H), | |
| | | 7.92 (bs, 1H), 8.96 (s, 1H), | |
| | | 9.35 (s, 1H), 10.57 (s, 1H), | |
| | | 11.42 (s, 1H). | |
| | | Isomer-4_ D2E2: ¹H NMR | |
| | | (400 MHz, DMSO-d₆): δ | |
| | | 0.803 (s, 3H), 0.829 (s, 3H), | |
| | | 1.23 (bs, 3H), 3.45 (s, 3H), | |
| | | 3.78 (s, 2H), 3.95-4.02 (m, | |
| | | 1H), 4.49 (bs, 1H), 4.85 (d, | |
| | | J = 10.4 Hz, 1H), 6.99 (s, 1H), | |
| | | 7.24 (s, 1H), 7.29 (d, J = 8.0 | |
| | | Hz, 1H), 7.44-7.46 (m, 2H), | |
| | | 7.92 (bs, 1H), 8.95 (s, 1H), | |
| | | 9.34 (s, 1H), 10.57 (s, 1H), | |
| | | 11.42 (s, 1H). | |
| Example 87 | 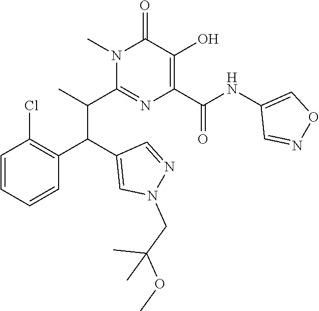 [SMILES: COC(C)(C)Cn1cc(C(c2ccccc2Cl)C(C)c2nc(C(=O)Nc3cnoc3)c(O)c(=O)n2C)cn1] | Isomer-1_ D1: ¹H NMR (400 MHz, DMSO-d₆): δ 1.16 (s, 6H), 1.31 (d, J = 4.8 Hz, 3H), 3.63 (s, 3H), 4.16 (s, 4H), 4.44 (s, 2H), 5.25-5.30 (m, 1H), 7.05-7.15 (m, 1H), 7.30-7.31 (m, 2H), 7.68 (d, J = 7.2 Hz, 1H), 8.61 (s, 1H), 8.66 (s, 1H), 8.88 (s, 1H), 9.30 (s, 1H). Isomer-1_ D2: ¹H NMR (400 MHz, DMSO-d₆): δ 1.16 (s, 6H), 1.31 (d, J = 4.8 Hz, 3H), 3.62 (s, 3H), 4.16 (s, 4H), 4.43 (s, 2H), 5.25-5.28 (m, 1H), 7.13-7.19 (m, 1H), 7.30-7.32 (m, 2H), 7.68 (d, J = 7.2 Hz, 1H), 8.61 (s, 1H), 8.66 (s, 1H), 8.88 (s, 1H), 9.30 (s, 1H). | Isomer- 1_(D1)_LCMS: m/ [symbol] 541.3 [M⁺ + 1]. Isomer- 2_(D2)_LCMS: m/ [symbol] 541.1 [M⁺ + 1]. HPLC: FR-1 (Isomer-1; D1): R_{T} = 4.65 (98%); FR-2 (Isomer-2; D2): R_{T} = 3.13 (99%). |
| Example 88 | 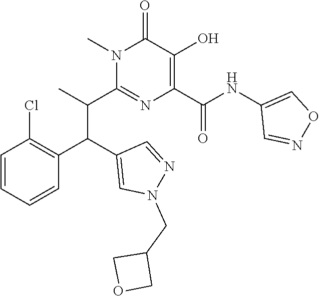 [SMILES: CC(c1nc(C(=O)Nc2cnoc2)c(O)c(=O)n1C)C(c1cnn(CC2COC2)c1)c1ccccc1Cl] | Isomer-1_ D1E1: ¹H NMR 3H), 4.61-4.62 (m, 1H), 5.12 (bs, 1H), 7.01-7.04 (m, 1H), 7.15-7.25 (m, 2H), 7.56 (bs, 1H), 7.64 (d, J = 7.2 Hz, 1H), 7.80 (s, 1H), 8.94 (s, 1H), 9.33 (s, 1H), 10.43 (s, 1H), 11.17 (s, 1H). Isomer-2_ D1E2: ¹H NMR (400 MHz, DMSO-d₆): δ 1.28 (bs, 3H), 2.34 (s, 2H), 3.23 (s, 1H), 3.61 (s, 3H), 4.03 (bs, 1H), 4.36-4.41 (m, 3H), 4.60-4.64 (m, 1H), 5.09 (bs, 1H), 7.02 (d, J = 7.2 Hz, 1H), 7.15-7.25 (m, 2H), 7.56 (bs, 1H), 7.63 (d, J = 7.6 Hz, 1H), 7.80 (s, 1H), 8.94 (s, 1H), 9.32 (s, 1H), 10.39 (s, | Isomer- 1_(D1E1)_LCMS: m/ [symbol] 525.3 [M⁺ + 1]. Isomer- 2_(D1E2)_LCMS: m/ [symbol] 525.1 [M⁺ + 1]. Isomer- 3_(D2E1)_LCMS: m/ [symbol] 525.3 [M⁺ + 1]. Isomer- 4_(D2E2)_LCMS: m/ [symbol] 525.1 [M⁺ + 1]. HPLC: FR-1 (Isomer-1; D1E1): R_{T} =4.37 (96%); FR-2 (Isomer-2; D1E2): R_{T} = 4.43 (100%); FR-3 (Isomer-3; D2E1): |
| | | (400 MHz, DMSO-d₆): δ | R_{T} = 4.59 (98%); |
| | | 1.27 (bs, 3H), 2.34 (s, 2H), | FR-4 (Isomer-4; |
| | | 3.23 (s, 1H), 3.61 (s, 3H), | D2E2): R_{T} = 4.63 |
| | | 4.02 (bs, 1H), 4.36-4.39 (m, | (100%). |
| | | 1H), 11.14 (s, 1H). | |
| | | Isomer-3_ D2E1: ¹H NMR | |
| | | (400 MHz, DMSO-d₆): ): 8 | |
| | | 1.21 (bs, 3H), 2.34 (s, 2H), | |
| | | 3.14-3.18 (m, 1H), 3.39 (s, | |
| | | 3H), 3.92 (bs, 1H), 4.20-4.21 | |
| | | (m, 3H), 4.44-4.49 (m, 1H), | |
| | | 4.83 (d, J = 10.4 Hz, 1H), | |
| | | 7.01 (s, 1H), 7.29-7.32 (m, | |
| | | 2H), 7.45-7.47 (m, 2H), 7.90 | |
| | | (bs, 1H), 8.95 (s, 1H), 9.35 | |
| | | (s, 1H), 10.64 (s, 1H), 11.46 | |
| | | (s, 1H). | |
| | | Isomer-4_ D2E2: ¹H NMR | |
| | | (400 MHz, DMSO-d₆): δ | |
| | | 1.21 (bs, 3H), 2.34 (s, 2H), | |
| | | 3.17-3.20 (m, 1H), 3.30 (s, | |
| | | 3H), 3.92 (bs, 1H), 4.19-4.21 | |
| | | (m, 3H), 4.43-4.49 (m, 1H), | |
| | | 4.82 (d, J = 11.2 Hz, 1H), | |
| | | 7.01 (s, 1H), 7.28-7.32 (m, | |
| | | 2H), 7.45-7.47 (m, 2H), 7.89 | |
| | | (bs, 1H), 8.95 (s, 1H), 9.34 | |
| | | (s, 1H), 10.52 (s, 1H), 11.46 | |
| | | (s, 1H). | |
| Example 89 | 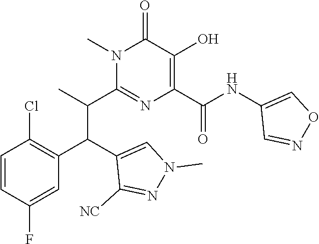 [SMILES: CC(c1nc(C(=O)Nc2cnoc2)c(O)c(=O)n1C)C(c1cc(F)ccc1Cl)c1cn(C)nc1C#N] | Isomer-1_D1E1: ¹H NMR (300 MHz, DMSO-d₆) δ 11.18 (s, 1H), 10.33 (s, 1H), 9.32 (s, 1H), 8.89 (s, 1H), 8.24 (s, 1H), 7.55 − 7.51 (m, 1H), 7.35 − 7.30 (m, 1H), 7.00 − 6.94 (m, 1H), 5.14(d, 1H), 4.11 − 4.05 (m, 1H), 3.95 (s, 3H), 3.62 (s, 3H), 1.34 (d, 3H). Isomer-2_D1E2: ¹H NMR (300 MHz, DMSO-d₆) δ 11.18 (s, 1H), 10.44 (s, 1H), 9.31 (s, 1H), 8.89 (s, 1H), 8.24 (s, 1H), 7.54 − 7.50 (m, | Isomer- 1_(D1E1)_LCMS: m/ [symbol] 509.8 [M⁻ − 1]. Isomer- 2_(D1E2)_LCMS: m/ [symbol] 509.8 [M⁻ − 1]. |
| | | 1H), 7.35 − 7.30 (m, 1H), | |
| | | 7.00-6.94 (m, 1H), 5.14(d, | |
| | | 1H), 4.09 − 4.04 (m, 1H), | |
| | | 3.95 (s, 3H), 3.61(s, 3H), | |
| | | 1.33 (d, 3H). | |
| Example 89 | 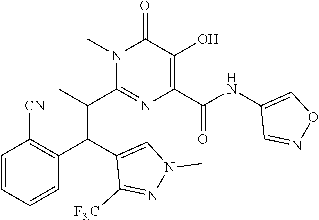 | DI1E1 ¹H NMR (400 MHz, DMSO d₆) δ 11.37 (s, 1H), 10.30 (s, 1H), 9.30 (s, 1H), 8.84 (s, 1H), 8.53 (s, 1H), 7.71 (d, 1H), 7.59 (d, 2H), 7.26 (t, 1H), 4.95 (d, 1H), 4.06 − 3.96 (m, 4H), 3.48 (s, 3H), 1.42 (d, 3H). D1E2 ¹H NMR (400 MHz, DMSO- d₆) δ 11.37 (s, 1H), 10.30 (s, 1H), 9.30 (s, 1H), 8.85 (s, 1H), 8.54 (s, 1H), 7.71 (d, | Isomer- 1_(D1E1)_LCMS: m/ [symbol] 528.3 [M⁺ + 1]. Isomer- 2_(D1E2)_LCMS: m/ [symbol] 528.3 [M⁺ + 1]. Isomer- 3_(D2E1)_LCMS: m/ [symbol] 528.3 [M⁺ + 1]. Isomer- 4_(D2E2)_LCMS: m/ [symbol] 528.3 [M⁺ + 1]. |
| | | 1H), 7.58 (t, 2H), 7.26 (t, | |
| | | 1H), 4.95 (d, 1H), 4.06 − | |
| | | 4.02 (m, 1H), 3.96 (s, | |
| | | 3H),3.48 (s, 3H), 1.42 (d, J = | |
| | | 6.4 Hz, 3H). | |
| | | D2E1 | |
| | | ¹H NMR (400 MHz, DMSO- | |
| | | d₆) δ 10.90 (s, 1H), 9.93 (s, | |
| | | 1H), 9.30 (s, 1H), 8.73 (s, | |
| | | 1H), 8.31 (s, 1H), 7.88 (d, | |
| | | 1H), 7.78 − 7.70 (m, 2H), | |
| | | 7.76 − 7.67 (m, 1H), 7.49 (t, | |
| | | 1H), 5.33 (d, 1H), 4.00 − | |
| | | 3.95 (m, 1H), 3.85 (s, 3H), | |
| | | 3.63 (s, 3H), 1.04 (d, 3H). | |
| | | D2E2 | |
| | | ¹H NMR (400 MHz, DMSO- | |
| | | d₆) δ 10.90 (s, 1H), 9.93 (s, | |
| | | 1H), 9.30 (s, 1H), 8.73 (s, | |
| | | 1H), 8.31 (s, 1H), 7.88 (d, | |
| | | 1H), 7.78 − 7.70 (m, 2H), | |
| | | 7.76 − 7.67 (m, 1H), 7.49 (t, | |
| | | 1H), 5.33 (d, 1H), 4.00 − | |
| | | 3.95 (m, 1H), 3.85 (s, 3H), | |
| | | 3.63 (s, 3H), 1.04 (d, 3H). | |
| Example 91 | 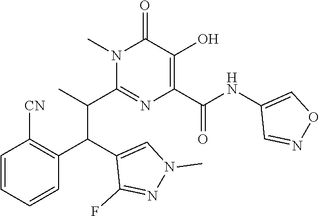 [SMILES: CC(c1nc(C(=O)Nc2cnoc2)c(O)c(=O)n1C)C(c1ccccc1C#N)c1cn(C)nc1F] | D1E1 ¹H NMR (400 MHz, DMSO- d₆) δ 11.25 (s, 1H), 10.44 (s, 1H), 9.29 (s, 1H), 8.86 (s, 1H), 7.90 (d, J = 2.4 Hz, 1H), 7.70 (d, J = 7.8 Hz, 1H), 7.65 − 7.52 (m, 2H), 7.24 (td, 1H), 4.93 (d, J = 11.1 Hz, 1H), 4.07-4.03 (m, 1H), 3.72 (s, 3H), 3.57 (s, 3H), 1.41 (d, 3H). D1E2 ¹H NMR (400 MHz, DMSO- d₆) δ 11.26 (s, 1H), 10.45 (s, | Isomer- 1_(D1E1)_LCMS: m/ [symbol] 478.0 [M⁺ + 1]. Isomer- 2_(D1E2)_LCMS: m/ [symbol] 478.0 [M⁺ + 1]. |
| | | 1H), 9.30 (s, 1H), 8.87 (s, | |
| | | 1H), 7.91 (d, J = 2.4 Hz, 1H), | |
| | | 7.81 − 7.44 (m, 3H), 7.24 (t, | |
| | | J = 7.6 Hz, 1H), 4.94 (d, J = | |
| | | 11.0 Hz, 1H), 4.08-4.03 (m, | |
| | | 1H), 3.73 (s, 3H), 3.58 (s, | |
| | | 3H), 1.42 (d, J = 6.5 Hz, 3H). | |
| Example 92 | 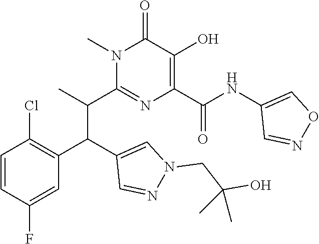 [SMILES: CC(c1nc(C(=O)Nc2cnoc2)c(O)c(=O)n1C)C(c1cnn(CC(C)(C)O)c1)c1cc(F)ccc1Cl] | D1E1 ¹H NMR (400 MHz, DMSO- d₆) δ 11.15 (s, 1H), 10.39 (s, 1H), 9.33 (s, 1H), 8.95 (s, 1H), 7.78 (s, 1H), 7.65 − 7.59 (m, 2H), 7.28 − 7.24 (m, 1H), 6.91 − 6.86 (m, 1H), 5.15 (d, 1H), 4.69 (s, 1H), 4.09 − 4.04 (m, 1H), 3.97 (s, 2H), 3.65 (s, 3H), 1.29 − 1.27 (d, 3H), 1.05 (s, 3H), 1.00 (s, 3H). D1E2 ¹H NMR (400 MHz, DMSO- d₆) δ 11.15 (s, 1H), 10.39 (s, 1H), 9.33 (s, 1H), 8.95 (s, | Isomer- 1_(D1E1)_LCMS: m/ [symbol] 545.2 [M⁺ + 1]. Isomer- 2_(D1E2)_LCMS: m/ [symbol] 545.2 [M⁺ + 1]. |
| | | 1H), 7.78 (s, 1H), 7.65 − 7.59 | |
| | | (m, 2H), 7.28 − 7.24 (m, 1H), | |
| | | 6.91 − 6.86 (m, 1H), 5.15 (d, | |
| | | 1H), 4.69 (s, 1H), 4.09 − 4.04 | |
| | | (m, 1H), 3.97 (s, 2H), 3.65 | |
| | | (s, 3H), 1.29 − 1.27 (d, 3H), | |
| | | 1.05 (s, 3H), 1.00 (s, 3H). | |
| Example 93 | 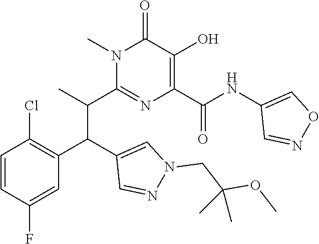 [SMILES: COC(C)(C)Cn1cc(C(c2cc(F)ccc2Cl)C(C)c2nc(C(=O)Nc3cnoc3)c(O)c(=O)n2C)cn1] | D1E1 ¹H NMR (400 MHz, DMSO- d₆) δ 11.14 (s, 1H), 10.37 (s, 1H), 9.33 (s, 1H), 8.95 (s, 1H), 7.73 (s, 1H), 7.64 − 7.58 (m, 2H), 7.28 − 7.24 (m, 1H), 6.91 − 6.86 (m, 1H), 5.14(d, 1H), 4.08 − 4.07 (m, 1H), 3.65 (s, 3H), 3.16 (s, 3H), 1.28 − 1.27 (d, 3H), 1.06(s, 3H), 1.02 (s, 3H). D1E2 ¹H NMR (400 MHz, DMSO- d₆) δ 11.14 (s, 1H), 10.37 (s, 1H), 9.33 (s, 1H), 8.95 (s, | Isomer- 1_(D1E1)_LCMS: m/ [symbol] 559.2 [M⁺ + 1]. Isomer- 2_(D1E2)_LCMS: m/ [symbol] 559.2 [M⁺ + 1]. |
| | | 1H), 7.73 (s, 1H), 7.64 − 7.58 | |
| | | (m, 2H), 7.28 − 7.24 (m, 1H), | |
| | | 6.91 − 6.86 (m, 1H), 5.14 (d, | |
| | | 1H), 4.08 − 4.07 (m, 1H), | |
| | | 3.65 (s, 3H), 3.16 (s, 3H), | |
| | | 1.28 − 1.27 (d, 3H), 1.06(s, | |
| | | 3H), 1.02 (s, 3H). | |
| Example 94 | 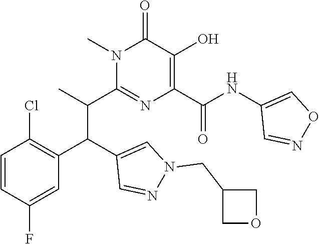 [SMILES: CC(c1nc(C(=O)Nc2cnoc2)c(O)c(=O)n1C)C(c1cnn(CC2COC2)c1)c1cc(F)ccc1Cl] | D1E1 ¹H NMR (400 MHz, DMSO- d₆) δ 11.13 (s, 1H), 10.36 (s, 1H), 9.32 (s, 1H), 8.94 (s, 1H), 7.83(s, 1H), 7.87 (s, 1H), 7.61-7.57 (m, 2H), 7.29-7.24 (m, 1H), 6.93-6.86 (m, 1H), 5.13 (d, 1H), 4.65- 4.60 (m, 2H), 4.41-4.37 (m, 4H), 4.07-4.01 (m, 1H), 3.65 (S, 3H), 3.42-3.37 (t, 1H), 1. 28-1.26 (d, 3H). D1E2 ¹H NMR (400 MHz, DMSO- d₆) δ 11.13 (s, 1H), 10.35 (s, | Isomer- 1_(D1E1)_LCMS: m/ [symbol] 543.3 [M⁺ + 1]. Isomer- 2_(D1E2)_LCMS: m/ [symbol] 543.3 [M⁺ + 1]. |
| | | 1H), 9.32 (s, 1H), 8.94 (s, | |
| | | 1H), 7.83(s, 1H), 7.87 (s, | |
| | | 1H), 7.61-7.57 (m, 2H), | |
| | | 7.29-7.24 (m, 1H), 6.93-6.86 | |
| | | (m, 1H), 5.12 (d, 1H), 4.65- | |
| | | 4.60 (m, 2H), 4.41-4.37 (m, | |
| | | 4H), 4.07-4.01 (m, 1H), 3.65 | |
| | | (S, 3H), 3.42-3.37 (t, 1H), 1. | |
| | | 28-1.26 (d, 3H).687 | |
| Example 95 | 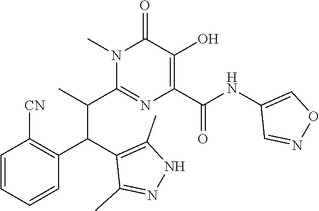 [SMILES: Cc1n[nH]c(C)c1C(c1ccccc1C#N)C(C)c1nc(C(=O)Nc2cnoc2)c(O)c(=O)n1C] | DI1E1 ¹H NMR (300 MHz, DMSO- d₆) δ 12.11 (s, 1H), 11.12 (s, 1H), 10.28 (s, 1H), 9.28 (s, 1H), 8.83 (s, 1H), 7.69 -7.47 (m, 3H), 7.26-7.20 (m, 1H), 4.97 (d, J = 11.3 Hz, 1H), 4.01-3.94 (m, 1H), 3.62 (s, 2H), 2.32-2.25 (m, 5H), 1.30 (d, J = 6.6 Hz, 28. D1E2 ¹H NMR (300 MHz, DMSO- d₆) δ 11.83-11.46 (m, 2H), 10.28 (s, 1H), 9.28 (s, 1H), | Isomer- 1_(D1E1)_LCMS: m/ [symbol] 474.0 [M⁺ + 1]. Isomer- 2_(D1E2)_LCMS: m/ [symbol] 474.0 [M⁺ + 1]. Isomer- 3_(D2E1)_LCMS: m/ [symbol] 474.0 [M⁺ + 1]. Isomer- 4_(D2E2)_LCMS: m/ [symbol] 474.0 [M⁺ + 1]. |
| | | 8.83 (s, 1H), 7.69 -7.47 (m, | |
| | | 3H), 7.26-7.20 (m, 1H), 4.97 | |
| | | (d, J = 11.3 Hz, 1H), 4.01- | |
| | | 3.94 (m, 1H), 3.62 (s, 2H), | |
| | | 2.32-2.26 (m, 5H), 1.30 (d, | |
| | | J = 6.6 Hz, 2H). | |
| | | D2E1 | |
| | | ¹H NMR (400 MHz, DMSO- | |
| | | d₆) δ 11.74 (brs, 2H), 10.81 | |
| | | (s, 1H), 9.34 (s, 1H), 8.94 (s, | |
| | | 1H), 8.18 -8.10 (m, 1H), | |
| | | 7.86 -7.73 (m, 2H), 7.45 (t, | |
| | | J = 7.5 Hz, 1H), 4.67 (d, J = | |
| | | 11.2 Hz, 1H), 4.33-4.28 (m, | |
| | | 1H), 3.36 (s, 3H), 1.90 (s, | |
| | | 6H), 1.41 (d, J = 6.2 Hz, 3H). | |
| | | D2E2 | |
| | | ¹H NMR (400 MHz, DMSO- | |
| | | d₆) δ 11.69 (brs, 2H), 10.79 | |
| | | (s, 1H), 9.34 (s, 1H), 8.94 (s, | |
| | | 1H), 8.18 -8.10 (m, 1H), | |
| | | 7.86 − 7.73 (m, 2H), 7.45 (t, | |
| | | J = 7.5 Hz, 1H), 4.67 (d, J = | |
| | | 11.2 Hz, 1H), 4.33-4.28 (m, | |
| | | 1H), 3.36 (s, 3H), 1.90 (s, | |
| | | 6H), 1.41 (d, J = 6.2 Hz, 3H). | |
| Example 96 | 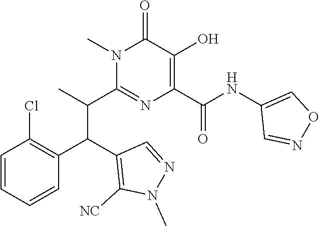 [SMILES: CC(c1nc(C(=O)Nc2cnoc2)c(O)c(=O)n1C)C(c1ccccc1Cl)c1cnn(C)c1C#N] | D1E1 ¹H NMR (400 MHz, DMSO- d₆) δ 11.08 (s, 1H), 10.32 (s, 1H), 9.25 (s, 1H), 8.84 (s, 1H), 7.97 (s, 1H), 7.55 (d, 1H), 7.22 − 7.10 (m, 2H), 7.02-6.98 (m, 1H), 5.16 (d, J = 10.9 Hz, 1H), 4.15-4.01 (m, 1H), 3.91 (s, 3H), 3.54 (s, 3H), 1.27 (d, J = 6.4 Hz, 3H). D1E2 ¹H NMR (400 MHz, DMSO- d₆) δ 11.05 (s, 1H), 10.32 (s, | Isomer- 1_(D1E1)_LCMS: m/ [symbol] 494.0 [M⁺ + 1]. Isomer- 2_(D1E2)_LCMS: m/ [symbol] 494.0 [M⁺ + 1]. Isomer- 3_(D2E1)_LCMS: m/ [symbol] 494.0 [M⁺ + 1]. Isomer- 4_(D2E2)_LCMS: m/ [symbol] 494.0 [M⁺ + 1]. |
| | | 1H), 9.25 (s, 1H), 8.84 (s, | |
| | | 1H), 7.97 (s, 1H), 7.55 (d, | |
| | | 1H), 7.19-7.14 (m, 2H), | |
| | | 7.02-6.98 (m, 1H), 5.17 (d, | |
| | | J = 11.0 Hz, 1H), 4.15-4.02 | |
| | | (m, 1H), 3.91 (s, 3H), 3.54 | |
| | | (s, 3H), 1.27 (d, J = 6.5 Hz, | |
| | | 3H). | |
| | | D2E1 | |
| | | ¹H NMR (400 MHz, DMSO- | |
| | | d₆) δ 11.27 (s, 1H), 10.47 (s, | |
| | | 1H), 9.31 (s, 1H), 8.86 (s, | |
| | | 1H), 8.00 (d, 1H), 7.79 (s, | |
| | | 1H), 7.54 − 7.37 (m, 2H), | |
| | | 7.36 − 7.28 (m, 1H), 5.22 (d, | |
| | | J = 11.1 Hz, 1H), 4.16-4.09 | |
| | | (m, 1H), 3.78 (s, 3H), 3.60 | |
| | | (s, 3H), 1.23 (d, J = 6.5 Hz, | |
| | | 3H). | |
| | | D2E2 | |
| | | ¹H NMR (400 MHz, DMSO- | |
| | | d₆) δ 11.28 (s, 1H), 10.46 (s, | |
| | | 1H), 9.31 (s, 1H), 8.86 (s, | |
| | | 1H), 8.07 − 7.90 (m, 1H), | |
| | | 7.80 (s, 1H), 7.53-7.38 (m, | |
| | | 2H), 7.36-7.28 (m, 1H), 5.23 | |
| | | (d, J = 11.1 Hz, 1H), 4.16- | |
| | | 4.09 (m, 1H), 3.78 (s, 3H), | |
| | | 3.61 (s, 3H), 1.23 (d, J = 6.5 | |
| | | Hz, 3H). | |
| Example 97 | 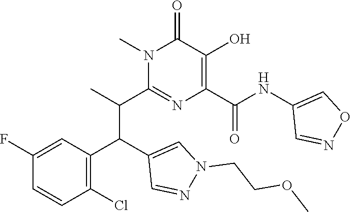 [SMILES: COCCn1cc(C(c2cc(F)ccc2Cl)C(C)c2nc(C(=O)Nc3cnoc3)c(O)c(=O)n2C)cn1] | D1E1 ¹H NMR (400 MHz, DMSO- d₆) δ 11.14 (s, 1H), 10.37(s, 1H), 9.33 (s, 1H), 8.95 (s, 1H), 7.79 (s, 1H), 7.62-7.59 (m, 2H), 7.28-7.24 (m, 1H), 6.91-6.86 (m, 1H), 5.12 (d, 1H), 4.22-4.20 (m, 2H), 4.07-4.03 (m, 1H), 3.67-3.64 (m, 5H), 3.19 (s, 3H), 1.28- 1.26 (d, 3H) D1E2 ¹H NMR (400 MHz, DMSO-d₆) δ 11.14 (s, 1H), 10.38(s, 1H), | Isomer- 1_(D1E1)_LCMS: m/ [symbol] 531.1 [M⁺ + 1]. Isomer- 2_(D1E2)_LCMS: m/ [symbol] 531.1 [M⁺ + 1]. |
| | | 9.33 (s, 1H), 8.95 (s, 1H), 7.79 | |
| | | (s, 1H), 7.62-7.59 (m, 2H), | |
| | | 7.28-7.24 (m, 1H), 6.90-6.86 | |
| | | (m, 1H), 5.12 (d, 1H), 4.22-4.20 | |
| | | (m, 2H), 4.07-4.03 (m, 1H), | |
| | | 3.67-3.64 (m, 5H), 3.19 (s, 3H), | |
| | | 1.28-1.26 (d, 3H). | |
| Example 98 | 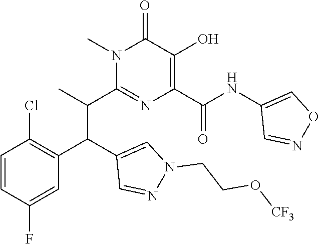 [SMILES: CC(c1nc(C(=O)Nc2cnoc2)c(O)c(=O)n1C)C(c1cnn(CCOC(F)(F)F)c1)c1cc(F)ccc1Cl] | D1E1 ¹H NMR (400 MHz, DMSO- d₆) δ 11.13 (s, 1H), 10.37 (s, 1H), 9.32 (s, 1H), 8.94 (s, 1H), 7.86 (s, 1H), 7.67 (s, 1H), 7.62 − 7.59 (m, 1H), 7.28 − 7.24 (m, 1H), 6.91 − 6.86 (m, 1H), 5.14 (d, 1H), 4.43-3.39 (m, 4H), 4.09 − 4.05 (m, 1H), 3.65 (s, 3H), 1.27 (d, 3H). D1E2 ¹H NMR (400 MHz, DMSO- d₆) δ 11.13 (s, 1H), 10.37 (s, 1H), 9.32 (s, 1H), 8.94 (s, | Isomer- 1_(D1E1)_LCMS: m/ [symbol] 585.0 [M⁺ + 1]. Isomer- 2_(D1E2)_LCMS: m/ [symbol] 585.0 [M⁺ + 1]. |
| | | 1H), 7.86 (s, 1H), 7.67 (s, | |
| | | 1H), 7.62 − 7.59 (m, 1H), | |
| | | 7.28 − 7.24 (m, 1H), 6.91 − | |
| | | 6.87 (m, 1H), 5.14 (d, 1H), | |
| | | 4.41 (t, 4H), 4.09 − 4.05 (m, | |
| | | 1H), 3.65 (s, 3H), 1.26 (d, | |
| | | 3H). | |
| Example 99 | 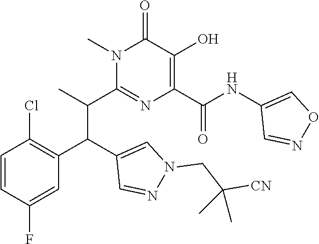 [SMILES: CC(c1nc(C(=O)Nc2cnoc2)c(O)c(=O)n1C)C(c1cnn(CC(C)(C)C#N)c1)c1cc(F)ccc1Cl] | D1E1 ¹H NMR (400 MHz, DMSO- d₆) δ 11.15 (s, 1H), 10.39 (s, 1H), 9.33 (s, 1H), 8.94 (s, 1H), 7.86 (s, 1H), 7.69 − 7.60 (m, 2H), 7.29 − 7.25 (m, 1H), 6.92 − 6.87 (m, 1H), 5.17 (d, 1H), 4.36 − 4.24 (m, 2H), 4.10 − 4.06 (m, 1H), 3.66 (s, 3H), 1.30 − 1.28 (m, 9H). D1E2 ¹H NMR (400 MHz, DMSO- d₆) δ 11.14 (s, 1H), 10.39 (s, 1H), 9.33 (s, 1H), 8.94 (s, 1H), 7.86 (s, 1H), 7.69 − 7.60 | Isomer- 1_(D1E1)_LCMS: m/ [symbol] 554.1 [M⁺ + 1]. Isomer- 2_(D1E2)_LCMS: m/ [symbol] 554.1 [M⁺ + 1]. |
| | | (m, 2H), 7.29 − 7.25 (m, 1H), | |
| | | 6.92 − 6.87 (m, 1H), 5.17 (d, | |
| | | 1H), 4.36 − 4.24 (m, 2H), | |
| | | 4.10 − 4.06 (m, 1H), 3.66 (s, | |
| | | 3H), 1.30 − 1.28 (m, 9H). | |
| Example 100 | 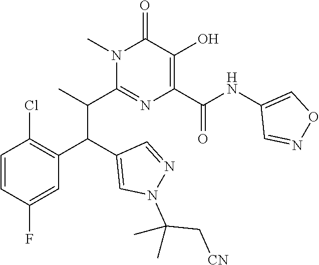 [SMILES: CC(c1nc(C(=O)Nc2cnoc2)c(O)c(=O)n1C)C(c1cnn(C(C)(C)CC#N)c1)c1cc(F)ccc1Cl] | D1E1 ¹H NMR (400 MHz, DMSO- d₆) δ 11.14 (s, 1H), 10.37 (s, 1H), 9.33 (d, 1H), 8.96 (d, 1H), 8.05 (s, 1H), 7.68-7.62 (m, 2H), 7.28 − 7.25 (m, 1H), 6.91 − 6.89 (m, 1H), 5.14 (d, 1H), 4.08 − 4.07 (m, 1H), 3.65 (s, 3H), 3.15 (s, 2H), 1.63 (d, 6H), 1.26 (d, 3H). D1E2 ¹H NMR (400 MHz, DMSO- d₆) δ 11.14 (s, 1H), 10.37 (s, 1H), 9.33 (s, 1H), 8.95 (s, 1H), 8.05 (s, 1H), 7.68 − 7.62 (m, 2H), 7.28 − 7.25 (m, 1H), 6.91 − 6.87 (m, 1H), 5.14 (d, | Isomer- 1_(D1E1)_LCMS: m/ [symbol] 554.1 [M⁺ + 1]. Isomer- 2_(D1E2)_LCMS: m/ [symbol] 554.1 [M⁺ + 1]. |
| | | 1H), 4.10 − 4.07 (m, 1H), | |
| | | 3.65 (s, 3H), 3.15 (s, 2H), | |
| | | 1.63 (d, 6H), 1.26 (d, 3H). | |
| Example 101 | 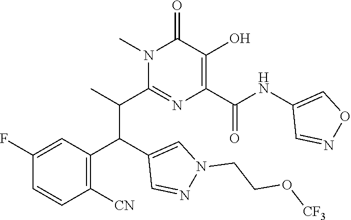 [SMILES: CC(c1nc(C(=O)Nc2cnoc2)c(O)c(=O)n1C)C(c1cnn(CCOC(F)(F)F)c1)c1cc(F)ccc1C#N] | D1E1 ¹H NMR (300 MHz, DMSO- d₆) δ 11.19 (s, 1H), 10.45 (s, 1H), 9.30 (s, 1H), 8.86 (s, 1H), 7.92 (s, 1H), 7.82 (dd, 1H), 7.79 − 7.70 (m, 2H), 7.14 − 7.07 (m, 1H), 5.06 (d, 1H), 4.43 − 4.40 (m, 4H), 4.14-4.07 (m, 1H), 3.63 (s, 3H), 1.30 (d, 3H). D1E2 ¹H NMR (300 MHz, DMSO- d₆) δ 11.19 (s, 1H), 10.45 (s, 1H), 9.30 (s, 1H), 8.86 (s, | Isomer- 1_(D1E1)_LCMS: m/ [symbol] 576.1 [M⁺ + 1]. Isomer- 2_(D1E2)_LCMS: m/ [symbol] 576.1 [M⁺ + 1]. |
| | | 1H), 7.92 (s, 1H), 7.82 (dd, | |
| | | 1H), 7.79 − 7.70 (m, 2H), | |
| | | 7.14 − 7.07 (m, 1H), 5.06 (d, | |
| | | 1H), 4.43 − 4.40 (m, 4H), | |
| | | 4.14-4.07 (m, 1H), 3.63 (s, | |
| | | 3H), 1.30 (d, 3H). | |
| Example 102 | 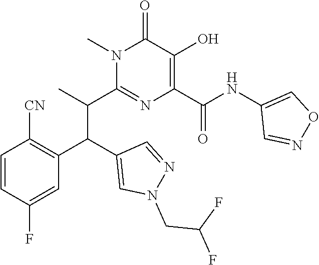 [SMILES: CC(c1nc(C(=O)Nc2cnoc2)c(O)c(=O)n1C)C(c1cnn(CC(F)F)c1)c1cc(F)ccc1C#N] | D1E1 ¹H NMR (300 MHz, DMSO- d₆) δ 11.19 (s, 1H), 10.44 (s, 1H), 9.30 (s, 1H), 8.86 (s, 1H), 7.92 (s, 1H), 7.85 − 7.71 (m, 3H), 7.11 (td, 1H), 6.53 − 6.15 (m, 1H), 5.09 (d, 1H), 4.63 (td, 2H), 4.15-4.09 (m, 1H), 3.64 (s, 3H), 1.30 (d, 3H). D1E2 ¹H NMR (300 MHz, DMSO- d₆) δ 11.19 (s, 1H), 10.44 (s, 1H), 9.30 (s, 1H), 8.86 (s, 1H), 7.92 (s, 1H), 7.85 − 7.71 (m, 3H), 7.11 (td, 1H), 6.53 − 6.15 (m, 1H), 5.09 (d, 1H), | Isomer- 1_(D1E1)_LCMS: m/ [symbol] 528.2 [M⁺ + 1]. Isomer- 2_(D1E2)_LCMS: m/ [symbol] 528.2 [M⁺ + 1]. |
| | | 4.63 (td, 2H), 4.15-4.09 (m, | |
| | | 1H), 3.64 (s, 3H), 1.30 (d, | |
| | | 3H). | |
| Example 103 | 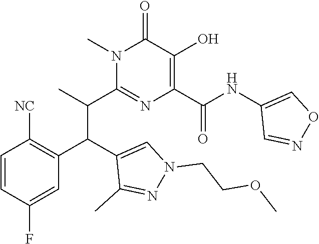 [SMILES: COCCn1cc(C(c2cc(F)ccc2C#N)C(C)c2nc(C(=O)Nc3cnoc3)c(O)c(=O)n2C)c(C)n1] | D1E1 ¹H NMR (400 MHz, DMSO- d₆) δ 11.23 (s, 1H), 10.39 (s, 1H), 9.30 (s, 1H), 8.84 (s, 1H), 7.99 (s, 1H), 7.77 − 7.66 (m, 2H), 7.12-7.07 (m, 1H), 4.90 (d, 1H), 4.14 (t, 2H), 4.03-3.99 (m, 1H), 3.65 (t, 2H), 3.60 (s, 3H), 3.20 (s, 3H), 2.30 (s, 3H), 1.31 (d, J = 6.5 Hz, 3H). D1E2 ¹H NMR (400 MHz, DMSO- d₆) δ 11.23 (s, 1H), 10.39 (s, 1H), 9.30 (s, 1H), 8.84 (s, | Isomer- 1_(D1E1)_LCMS: m/ [symbol] 536.2 [M⁺ + 1]. Isomer- 2_(D1E2)_LCMS: m/ [symbol] 536.2 [M⁺ + 1]. Isomer- 3_(D2E1)_LCMS: m/ [symbol] 536.2 [M⁺ + 1]. Isomer- 4_(D2E2)_LCMS: m/ [symbol] 536.2 [M⁺ + 1]. |
| | | 1H), 7.99 (s, 1H), 7.79 − 7.63 | |
| | | (m, 2H), 7.12-7.07 (m, 1H), | |
| | | 4.96 − 4.81 (m, 1H), 4.14 (t, | |
| | | J = 5.3 Hz, 2H), 4.03-3.99 | |
| | | (m, 1H), 3.65 (t, J = 5.4 Hz, | |
| | | 2H), 3.60 (s, 3H), 3.20 (s, | |
| | | 3H), 2.30 (s, 3H), 1.31 (d, J = | |
| | | 6.5 Hz, 3H). | |
| | | D2E1 | |
| | | ¹H NMR (400 MHz, DMSO- | |
| | | d₆) δ 11.11 (s, 1H), 10.47 (s, | |
| | | 1H), 9.33 (s, 1H), 8.85 (s, | |
| | | 1H), 7.98-7.92 (m, 2H), 7.69 | |
| | | (s, 1H), 7.37-7.32 (m, 1H), | |
| | | 4.79 (d, J = 10.8 Hz, 1H), | |
| | | 4.11 − 3.95 (m, 3H), 3.62 − | |
| | | 3.42 (m, 5H), 3.10 (s, 3H), | |
| | | 1.92 (s, 3H), 1.21 (d, J = 6.5 | |
| | | Hz, 3H). | |
| | | D2E2 | |
| | | ¹H NMR (400 MHz, DMSO- | |
| | | d₆) δ 11.11 (s, 1H), 10.48 (s, | |
| | | 1H), 9.33 (s, 1H), 8.85 (s, | |
| | | 1H), 7.98-7.92 (m, 2H), 7.69 | |
| | | (s, 1H), 7.37 − 7.31 (m, 1H), | |
| | | 4.79 (d, J = 10.7 Hz, 1H), | |
| | | 4.13 − 3.96 (m, 3H), 3.57 − | |
| | | 3.47 (m, 5H), 3.10 (s, 3H), | |
| | | 1.92 (s, 3H), 1.21 (d, J = 6.5 | |
| | | Hz, 4H). | |
| Example 104 | 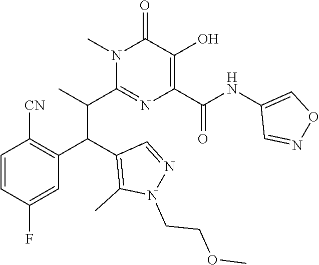 [SMILES: COCCn1ncc(C(c2cc(F)ccc2C#N)C(C)c2nc(C(=O)Nc3cnoc3)c(O)c(=O)n2C)c1C] | D1E1 ¹H NMR (400 MHz, DMSO- d₆) δ 11.19 (s, 1H), 10.41 (s, 1H), 9.30 (s, 1H), 8.84 (s, 1H), 7.86 − 7.76 (m, 2H), 7.70 (dd, 1H), 7.09 (td, 1H), 4.93 (d, J = 11.0, 1H), 4.16 (t, J = 5.5 Hz, 2H), 4.10 − 3.96 (m, 1H), 3.64-3.62 (m, 5H), 3.18 (s, 3H), 2.45 (s, 3H), 1.25 (d, J = 6.5 Hz, 3H). D1E2 ¹H NMR (400 MHz, DMSO- d₆) δ 11.19 (s, 1H), 10.41 (s, 1H), 9.30 (s, 1H), 8.84 (s, 1H), 7.87 − 7.75 (m, 2H), 7.70 (dd, 1H), 7.09 (td, 1H), | Isomer- 1_(D1E1)_LCMS: m/ [symbol] 536.2 [M⁺ + 1]. Isomer- 2_(D1E2)_LCMS: m/ [symbol] 536.2 [M⁺ + 1]. Isomer- 3_(D2E1)_LCMS: m/ [symbol] 536.2 [M⁺ + 1]. Isomer- 4_(D2E2)_LCMS: m/ [symbol] 536.2 [M⁺ + 1]. |
| | | 4.98 − 4.85 (m, 1H), 4.16 (t, | |
| | | J = 5.5 Hz, 2H), 4.10 − 3.96 | |
| | | (m, 1H), 3.64-3.62 (s, 5H), | |
| | | 3.18 (s, 3H), 2.45 (s, 3H), | |
| | | 1.25 (d, J = 6.5 Hz, 3H). | |
| | | D2E1 | |
| | | ¹H NMR (400 MHz, DMSO- | |
| | | d₆) δ 11.20 (s, 1H), 10.62 (s, | |
| | | 1H), 9.34 (s, 1H), 8.88 (s, | |
| | | 1H), 8.10 (dd, 1H), 7.93 (dd, | |
| | | 1H), 7.46 (s, 1H), 7.34 (td, | |
| | | 1H), 4.77 (d, J = 10.8 Hz, | |
| | | 1H), 4.17-4.11 (m, 1H), | |
| | | 3.97-3.92 (m, 2H), 3.49(s, | |
| | | 3H), 3.41-3.35 (m, 2H), 2.95 | |
| | | (s, 3H), 2.05 (s, 3H), 1.25 (d, | |
| | | J = 6.5 Hz, 3H). | |
| | | D2E2 | |
| | | ¹H NMR (400 MHz, DMSO- | |
| | | d₆) δ 11.19 (s, 1H), 10.60 (s, | |
| | | 1H), 9.34 (s, 1H), 8.88 (s, | |
| | | 1H), 8.16 − 8.03 (m, 1H), | |
| | | 7.93 (dd, 1H), 7.46 (s, 1H), | |
| | | 7.34 (td, 1H), 4.77 (d, J = | |
| | | 10.9 Hz, 1H), 4.17-4.11 (m, | |
| | | 1H), 3.97-3.92 (m, 2H), | |
| | | 3.49(s, 3H), 3.41-3.36 (m, | |
| | | 2H), 2.95 (s, 3H), 2.05 (s, | |
| | | 3H), 1.25 (d, J = 6.5 Hz, 3H). | |
| Example 105 | 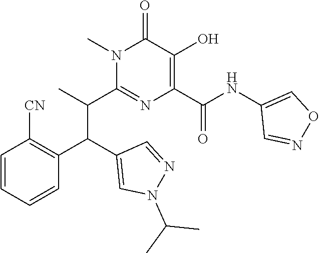 [SMILES: CC(c1nc(C(=O)Nc2cnoc2)c(O)c(=O)n1C)C(c1cnn(C(C)C)c1)c1ccccc1C#N] | D1E1 ¹H NMR (400 MHz, DMSO- d₆) δ 11.17 (s, 1H), 10.43 (s, 1H), 9.30 (s, 1H), 8.88 (s, 1H), 7.83 (t, 2H), 7.62 − 7.52 (m, 3H), 7.21 (t, 1H), 4.98 (d, 1H), 4.50 − 4.43 (m, 1H), 4.12 − 4.06 (m, 1H), 3.60 (s, 3H), 1.39 (d, 6H), 1.33 (d, 3H). D1E2 ¹H NMR (400 MHz, DMSO- d₆) δ 11.17 (s, 1H), 10.43 (s, 1H), 9.30 (s, 1H), 8.88 (s, 1H), 7.83 (t, 2H), 7.62 − 7.52 (m, 3H), 7.21 (t, 1H), 4.98 | Isomer- 1_(D1E1)_LCMS: m/ [symbol] 488.3 [M⁺ + 1]. Isomer- 2_(D1E2)_LCMS: m/ [symbol] 488.3 [M⁺ + 1]. Isomer- 3_(D2E1)_LCMS: m/ [symbol] 488.3 [M⁺ + 1]. Isomer- 4_(D2E2)_LCMS: m/ [symbol] 488.3 [M⁺ + 1]. |
| | | (d, 1H), 4.50 − 4.43 (m, 1H), | |
| | | 4.10 − 4.06 (m, 1H), 3.60 (s, | |
| | | 3H), 1.39 (d, 6H), 1.33 (d, | |
| | | 3H). | |
| | | D2E1 | |
| | | ¹H NMR (400 MHz, DMSO- | |
| | | d₆) δ 11.29 (s, 1H), 10.43 (s, | |
| | | 1H), 9.34 (s, 1H), 8.91 (s, | |
| | | 1H), 8.00 (d, 1H), 7.83 (q, | |
| | | 2H), 7.49 (t, 1H), 7.38 (s, | |
| | | 1H), 7.07 (s, 1H), 4.75 (d, | |
| | | 1H), 4.35 − 4.28 (m, 1H), | |
| | | 4.05 − 4.00 (m, 1H), 3.41 (s, | |
| | | 3H), 1.22 − 1.20 (m, 9H). | |
| | | D2E2 | |
| | | ¹H NMR (400 MHz, DMSO- | |
| | | d₆) δ 11.28 (s, 1H), 10.44 (s, | |
| | | 1H), 9.35 (s, 1H), 8.91 (s, | |
| | | 1H), 8.00 (d, 1H), 7.83 (q, | |
| | | 2H), 7.49 (t, 1H), 7.39 (s, | |
| | | 1H), 7.07 (s, 1H), 4.75 (d, | |
| | | 1H), 4.35 − 4.28 (m, 1H), | |
| | | 4.05 − 4.00 (m, 1H), 3.41 (s, | |
| | | 3H), 1.22 − 1.20 (m, 9H). | |
| Example 106 | 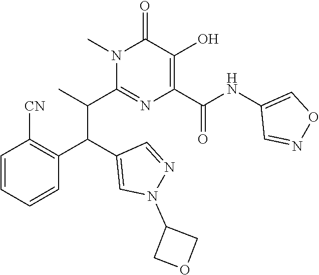 [SMILES: CC(c1nc(C(=O)Nc2cnoc2)c(O)c(=O)n1C)C(c1cnn(C2COC2)c1)c1ccccc1C#N] | D1E1 ¹H NMR (300 MHz, DMSO- d₆) δ 11.18 (s, 1H), 10.45 (s, 1H), 9.30 (s, 1H), 8.88 (s, 1H), 8.01 (s, 1H), 7.84 (d, 1H), 7.77 (s, 1H), 7.61 − 7.55 (m, 2H), 7.22 (t, 1H), 5.60 − 5.56 (m, 1H), 5.00 (d, 1H), 4.92 − 4.82 (m, 4H), 4.14 − 4.09 (m, 1H), 3.61 (s, 3H), 1.34 (d, 3H). D1E2 ¹H NMR (300 MHz, DMSO- d₆) δ 11.20 (s, 1H), 10.55 (s, 1H), 9.30 (s, 1H), 8.87 (s, 1H), 8.01 (s, 1H), 7.81 (d, 1H), 7.77 (s, 1H), 7.61 − 7.55 | Isomer- 1_(D1E1)_LCMS: m/ [symbol] 502.0 [M⁺ + 1]. Isomer- 2_(D1E2)_LCMS: m/ [symbol] 502.0 [M⁺ + 1]. Isomer- 3_(D2E1)_LCMS: m/ [symbol] 502.0 [M⁺ + 1]. Isomer- 4_(D2E2)_LCMS: m/ [symbol] 502.0 [M⁺ + 1]. |
| | | (m, 2H), 7.22 (t, 1H), 5.63 − | |
| | | 5.53 (m, 1H), 5.01 (d, 1H), | |
| | | 4.92 − 4.82 (m, 4H), 4.14 − | |
| | | 4.06 (m, 1H), 3.61 (s, 3H), | |
| | | 1.32 (d, 3H). | |
| | | D2E1 | |
| | | ¹H NMR (300 MHz, DMSO- | |
| | | d₆) δ 11.23 (s, 1H), 10.36 (s, | |
| | | 1H), 9.34 (s, 1H), 8.88 (s, | |
| | | 1H), 7.97 (d, 1H), 7.86 − | |
| | | 7.78 (m, 2H), 7.60 (s, 1H), | |
| | | 7.49 (t, 1H), 7.31 (s, 1H), | |
| | | 5.43 (t, 1H), 4.89 (d, 1H), | |
| | | 4.78 (m, 2H), 4.69 (t, 2H), | |
| | | 4.09 − 4.03 (m, 1H), 3.49 (s, | |
| | | 3H), 1.17 (d, 3H). | |
| | | D2E2 | |
| | | ¹H NMR (300 MHz, DMSO- | |
| | | d₆) δ 11.23 (s, 1H), 10.39 (s, | |
| | | 1H), 9.34 (s, 1H), 8.88 (s, | |
| | | 1H), 7.97 (d, 1H), 7.86 − | |
| | | 7.78 (m, 2H), 7.60 (s, 1H), | |
| | | 7.49 (t, 1H), 7.31 (s, 1H), | |
| | | 5.43 (t, 1H), 4.89 (d, 1H), | |
| | | 4.81 − 4.75 (m, 2H), 4.69 (t, | |
| | | 2H), 4.08 − 4.03 (m, 1H), | |
| | | 3.49 (s, 3H), 1.18 (d, 3H). | |
| Example 107 | 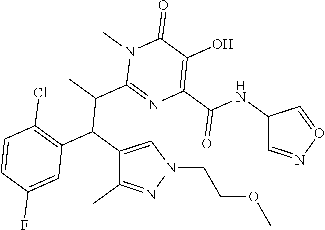 | D1E1 ¹H NMR (300 MHz, DMSO- d₆) δ 11.17 (s, 1H), 10.37 (s, 1H), 9.34 (s, 1H), 8.94 (s, 1H), 7.88 (s, 1H), 7.56 (d, 1H), 7.25 (dd, 1H), 6.89 (t, 1H), 4.95 (d, 1H), 4.13 (t, 2H), 3.96-3.92 (m, 1H), 3.67-3.61 (m, 5H), 3.20 (s, 3H), 2.30 (s, 3H), 1.28 (d, 3H). D1E2 1H NMR (400 MHz, DMSO- d6) δ 11.17 (s, 1H), 10.36 (s, | Isomer- 1_(D1E1)_LCMS: m/ [symbol] 545.2 [M⁺ + 1]. Isomer- 2_(D1E2)_LCMS: m/ [symbol] 545.2 [M⁺ + 1]. Isomer- 3_(D2E1)_LCMS: m/ [symbol] 545.2 [M⁺ + 1]. Isomer- 4_(D2E2)_LCMS: m/ [symbol] 545.2 [M⁺ + 1]. |
| | | 1H), 9.34 (s, 1H), 8.94 (s, | |
| | | 1H), 7.88 (s, 1H), 7.56 (d, | |
| | | 1H), 7.25 (dd, 1H), 6.89 (t, | |
| | | 1H), 4.95 (d, 1H), 4.13 (t, | |
| | | 2H), 3.96-3.92 (m, 1H), | |
| | | 3.67-3.61 (m, 5H), 3.20 (s, | |
| | | 3H), 2.30 (s, 3H), 1.28 (d, | |
| | | 3H). | |
| | | D2E1 | |
| | | ¹H NMR (400 MHz, DMSO- | |
| | | d₆) δ 11.28 (s, 1H), 10.60 (s, | |
| | | 1H), 9.34 (s, 1H), 8.92 (s, | |
| | | 1H), 7.86 (d, 1H), 7.59 (s, | |
| | | 1H), 7.48 (dd, 1H), 7.11 − | |
| | | 7.06 (m, 1H), 4.80 (d, 1H), | |
| | | 4.00 − 3.93 (m, 3H), 3.53 − | |
| | | 3.33 (m, 5H), 3.08 (s, 3H), | |
| | | 1.91 (s, 3H), 1.24 (d, 3H). | |
| | | D2E2 | |
| | | ¹H NMR (400 MHz, DMSO- | |
| | | d₆) δ 11.28 (s, 1H), 10.60 (s, | |
| | | 1H), 9.34 (s, 1H), 8.92 (s, | |
| | | 1H), 7.86 (d, 1H), 7.59 (s, | |
| | | 1H), 7.48 (dd, 1H), 7.13 (td, | |
| | | 1H), 4.80 (d, 1H), 3.99 − | |
| | | 3.90 (m, 3H), 3.53 − 3.42 (m, | |
| | | 5H), 3.08 (s, 3H), 1.91 (s, | |
| | | 3H), 1.23 (d, 3H). | |
| Example 108 | 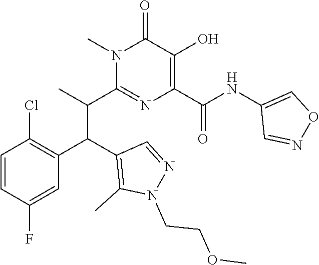 [SMILES: COCCn1ncc(C(c2cc(F)ccc2Cl)C(C)c2nc(C(=O)Nc3cnoc3)c(O)c(=O)n2C)c1C] | D1E1 ¹H NMR (400 MHz, DMSO- d₆) δ 11.13 (s, 1H), 10.36 (s, 1H), 9.33 (s, 1H), 8.93 (s, 1H), 7.68 (s, 1H), 7.63 (dd, 1H), 7.24 (dd, 1H), 6.90 − 6.85 (m, 1H), 4.97 (dd, 1H), 4.15 (t, 2H), 3.99 (dd, 1H), 3.63 (d, 5H), 3.18 (s, 3H), 2.44 (s, 3H), 1.22 (d, 3H). D1E2 ¹H NMR (400 MHz, DMSO- d₆) δ 11.13 (s, 1H), 10.39 (s, 1H), 9.33 (s, 1H), 8.93 (s, 1H), 7.68 (s, 1H), 7.63 (dd, 1H), 7.24 (dd, 1H), 6.88 (td, 1H), 4.98 (d, 1H), 4.15 (t, | Isomer- 1_(D1E1)_LCMS: Isomer- m/ [symbol] 545.2 [M⁺ + 1]. 2_(D1E2)_LCMS: m/ [symbol] 545.2 [M⁺ + 1]. Isomer- 3_(D2E1)_LCMS: m/ [symbol] 545.2 [M⁺ + 1]. Isomer- 4_(D2E2)_LCMS: m/ [symbol] 545.2 [M⁺ + 1]. |
| | | 2H), 4.01-3.96 (m, 1H), | |
| | | 3.65-3.60 (m, 5H), 3.18 (s, | |
| | | 3H), 2.44 (s, 3H), 1.22 (d, | |
| | | 3H). | |
| | | D2E1 | |
| | | ¹H NMR (400 MHz, DMSO- | |
| | | d₆) δ 11.37 (s, 1H), 10.70 (s, | |
| | | 1H), 9.35 (s, 1H), 8.94 (s, | |
| | | 1H), 7.98 (d, 1H), 7.47 (dd, | |
| | | 1H), 7.39 (s, 1H), 7.13 (td, | |
| | | 1H), 4.79 (d, 1H), 4.08-4.04 | |
| | | (m, 1H), 3.99 − 3.88 (m, 2H), | |
| | | 3.52 (s, 3H), 3.42 − 3.40 (m, | |
| | | 2H), 2.94 (s, 3H), 2.04 (s, | |
| | | 3H), 1.26 (d, 3H). | |
| | | D2E2 | |
| | | ¹H NMR (400 MHz, DMSO- | |
| | | d₆) δ 11.37 (s, 1H), 10.69 (s, | |
| | | 1H), 9.35 (s, 1H), 8.95 (s, | |
| | | 1H), 7.98 (d, 1H), 7.47 (dd, | |
| | | 1H), 7.39 (s, 1H), 7.13 (td, | |
| | | 1H), 4.79 (d, 1H), 4.06 (s, | |
| | | 1H), 3.99 − 3.88 (m, 2H), | |
| | | 3.52 (s, 3H), 3.42 − 3.40 (m, | |
| | | 2H), 2.94 (s, 3H), 2.04 (s, | |
| | | 3H), 1.26 (d, 3H). | |
| Example 109 | 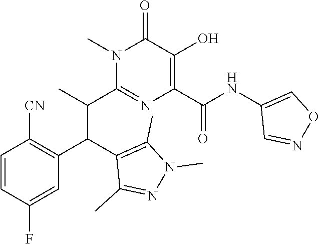 [SMILES: Cc1nn(C)c(C)c1C(c1cc(F)ccc1C#N)C(C)c1nc(C(=O)Nc2cnoc2)c(O)c(=O)n1C] | D1E1 ¹H NMR (400 MHz, DMSO- d₆) δ 11.06 (s, 1H), 10.26 (s, 1H), 9.28 (s, 1H), 8.80 (s, 1H), 7.75 (q, 1H), 7.43 − 7.40 (m, 1H), 7.14 − 7.09 (m, 1H), 5.03 (d, 1H), 3.94 − 3.90 (m, 1H), 3.65 (d, 6H), 2.40 (s, 3H), 2.20 (s, 3H), 1.24 (d, 3H). D1E2 ¹H NMR (400 MHz, DMSO- d₆) δ 11.06 (s, 1H), 10.26 (s, 1H), 9.28 (s, 1H), 8.80 (s, 1H), 7.75 (q, 1H), 7.43 − 7.40 (m, 1H), 7.14 − 7.09 (m, | Isomer- 1_(D1E1)_LCMS: m/ [symbol] 506.3 [M⁺ + 1]. Isomer- 2_(D1E2)_LCMS: m/ [symbol] 506.3 [M⁺ + 1]. |
| | | 1H), 5.03 (d, 1H), 3.94 − 3.90 | |
| | | (m, 1H), 3.65 (d, 6H), 2.40 | |
| | | (s, 3H), 2.20 (s, 3H), 1.24 (d, | |
| | | 3H). | |
| Example 110 | 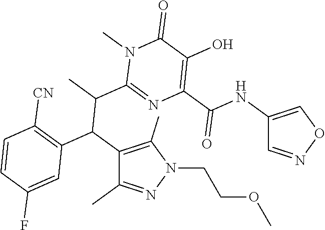 [SMILES: COCCn1nc(C)c(C(c2cc(F)ccc2C#N)C(C)c2nc(C(=O)Nc3cnoc3)c(O)c(=O)n2C)c1C] | D1E1 ¹H NMR (400 MHz, DMSO- d₆) δ 11.06 (s, 1H), 10.23 (s, 1H), 9.28 (s, 1H), 8.80 (s, 1H), 7.75 (q, 1H), 7.42 (q, 1H), 7.15 − 7.10 (m, 1H), 5.04 (d, 1H), 4.11 − 4.09 (m, 2H), 3.94 − 3.89 (m, 1H), 3.66 (s, 3H), 3.60 (t, 2H), 3.18 (s, 3H), 2.43 (s, 3H), 2.20 (s, 3H), 1.24 (d, 3H). D1E2 ¹H NMR (400 MHz, DMSO- d₆) δ 11.06 (s, 1H), 10.23 (s, 1H), 9.28 (s, 1H), 8.80 (s, | Isomer- 1_(D1E1)_LCMS: m/ [symbol] 550.3 [M⁺ + 1]. Isomer- 2_(D1E2)_LCMS: m/ [symbol] 550.3 [M⁺ + 1]. |
| | | 1H), 7.76 (q, 1H), 7.42 (q, | |
| | | 1H), 7.15 − 7.10 (m, 1H), | |
| | | 5.04 (d, 1H), 4.11 − 4.09 (m, | |
| | | 2H), 3.94 − 3.90 (m, 1H), | |
| | | 3.67 (s, 3H), 3.60 (t, 2H), | |
| | | 3.19 (s, 3H), 2.44 (s, 3H), | |
| | | 2.20 (s, 3H), 1.24 (d, 3H). | |
| Example 111 | 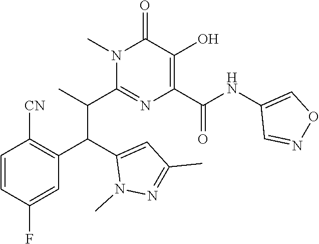 [SMILES: Cc1cc(C(c2cc(F)ccc2C#N)C(C)c2nc(C(=O)Nc3cnoc3)c(O)c(=O)n2C)n(C)n1] | D1E1 ¹H NMR (400 MHz, DMSO- d₆) δ 11.23 (s, 1H), 10.54 (s, 1H), 9.30 (s, 1H), 8.83 (s, 1H), 7.84 (d, 1H), 7.75 (dd, 1H), 7.16 (t, 1H), 6.51 (s, 1H), 5.20 (d, J = 10.8 Hz, 1H), 4.12-4.07 (m, 1H), 3.97 (s, 3H), 3.59 (s, 3H), 2.13 (s, 3H), 1.32 (d, J = 6.5 Hz, 3H). D1E2 ¹H NMR (400 MHz, DMSO- d₆) δ 11.23 (s, 1H), 10.54 (s, 1H), 9.30 (s, 1H), 8.83 (s, 1H), 7.84 (d, 1H), 7.75 (dd, | Isomer- 1_(D1E1)_LCMS: m/ [symbol] 492.2 [M⁺ + 1]. Isomer- 2_(D1E2)_LCMS: m/ [symbol] 492.2 [M⁺ + 1]. Isomer- 3_(D2E1)_LCMS: m/ [symbol] 492.2 [M⁺ + 1]. Isomer- 4_(D2E2)_LCMS: m/ [symbol] 492.2 [M⁺ + 1]. |
| | | 1H), 7.16 (t, 1H), 6.51 (s, | |
| | | 1H), 5.20 (d, J = 10.9 Hz, | |
| | | 1H), 4.12-4.06 (m, 1H), 3.97 | |
| | | (s, 3H), 3.59 (s, 3H), 2.13 (s, | |
| | | 3H), 1.32 (d, J = 6.5 Hz, 3H). | |
| | | D2E1 | |
| | | ¹H NMR (400 MHz, DMSO- | |
| | | d₆) δ 11.11 (s, 1H), 10.50 (s, | |
| | | 1H), 9.31 (s, 1H), 8.81 (s, | |
| | | 1H), 8.01 (dd, 1H), 7.91 (dd, | |
| | | 1H), 7.41 (t, 1H), 6.24 (s, | |
| | | 1H), 5.14 (d, J = 10.4 Hz, | |
| | | 1H), 4.14-4.09 (m, 1H), 3.66 | |
| | | (s, 3H), 3.64 (s, 3H), 1.98 (s, | |
| | | 3H), 1.15 (d, J = 6.5 Hz, 3H). | |
| | | D2E2 | |
| | | ¹H NMR (400 MHz, DMSO- | |
| | | d₆) δ 11.11 (s, 1H), 10.50 (s, | |
| | | 1H), 9.31 (s, 1H), 8.82 (s, | |
| | | 1H), 8.01 (dd, 1H), 7.90 (d, | |
| | | 1H), 7.41(t, 1H), 6.25 (s, | |
| | | 1H), 5.14 (d, J = 10.7 Hz, | |
| | | 1H), 4.14-4.09 (m, 1H), 3.66 | |
| | | (s, 3H), 3.64 (s, 3H), 1.98 (s, | |
| | | 3H), 1.15 (d, J = 6.5 Hz, 3H). | |
| Example 112 | 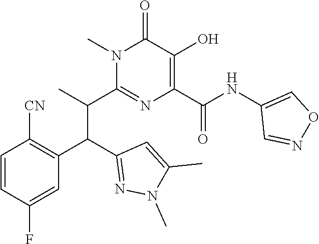 [SMILES: Cc1cc(C(c2cc(F)ccc2C#N)C(C)c2nc(C(=O)Nc3cnoc3)c(O)c(=O)n2C)nn1C] | D1E1 ¹H NMR (400 MHz, DMSO- d₆) δ 11.25 (s, 1H), 10.42 (s, 1H), 9.30 (s, 1H), 8.87 (s, 1H), 7.76 − 7.67 (m, 2H), 7.10 (t, 1H), 6.33 (s, 1H), 5.08 (d, J = 11.0 Hz, 1H), 4.11-4.06 (m, 1H), 3.68(s, 3H), 3.60 (s, 3H), 2.23 (s, 3H), 1.31 (d, J = 6.8, 3H). D1E2 ¹H NMR (400 MHz, DMSO- d₆) δ 11.25 (s, 1H), 10.41 (s, 1H), 9.30 (s, 1H), 8.87 (s, 1H), 7.89 − 7.66 (m, 2H), | Isomer- 1_(D1E1)_LCMS: m/ [symbol] 492.2 [M⁺ + 1]. Isomer- 2_(D1E2)_LCMS: m/ [symbol] 492.2 [M⁺ + 1]. Isomer- 3_(D2E1)_LCMS: m/ [symbol] 492.2 [M⁺ + 1]. Isomer- 4_(D2E2)_LCMS: m/ [symbol] 492.2 [M⁺ + 1]. |
| | | 7.09 (t, 1H), 6.33 (s, 1H), | |
| | | 5.08 (d, J = 11.0 Hz, 1H), | |
| | | 4.11-4.06 (m, 1H), 3.68(s, | |
| | | 3H), 3.60 (s, 3H), 2.23 (s, | |
| | | 3H), 1.31 (d, J = 6.8, 3H). | |
| | | D2E1 | |
| | | ¹H NMR (400 MHz, DMSO- | |
| | | d₆) δ 11.56 (brs, 1H), 9.27 (s, | |
| | | 1H), 8.80 (s, 1H), 7.97-7.92 | |
| | | (m, 1H), 7.68-7.63 (m, 1H), | |
| | | 7.36-7.31 (m, 1H), 5.76 (s, | |
| | | 1H), 4.99 (d, J = 10.4 Hz, | |
| | | 1H), 4.09-4.03 (m, 1H), 3.62 | |
| | | (s, 3H), 3.52 (s, 3H), 2.06 (s, | |
| | | 3H), 1.03 (d, 3H). | |
| | | D2E2 | |
| | | ¹H NMR (400 MHz, DMSO- | |
| | | d₆) δ 11.10 (s, 1H), 9.28 (s, | |
| | | 1H), 8.80 (s, 1H), 7.96-7.91 | |
| | | (m, 1H), 7.68-7.62 (m, 1H), | |
| | | 7.37-7.32 (m, 1H), 5.76 (s, | |
| | | 1H), 4.98 (d, J = 10.4 Hz, | |
| | | 1H), 4.09-4.03 (m, 1H), 3.62 | |
| | | (s, 3H), 3.52 (s, 3H), 2.06 (s, | |
| | | 3H), 1.03 (d, 3H). | |
| Example 113 | 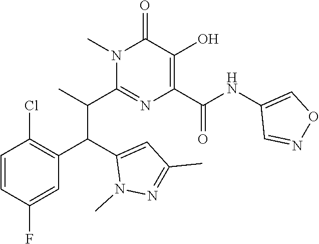 [SMILES: Cc1cc(C(c2cc(F)ccc2Cl)C(C)c2nc(C(=O)Nc3cnoc3)c(O)c(=O)n2C)n(C)n1] | D1E1 ¹H NMR (300 MHz, DMSO- d₆) δ 11.17 (brs, 1H), 10.53 (s, 1H), 9.33 (s, 1H), 8.93 (s, 1H), 7.63 (dd, 1H), 7.28 (dd, 1H), 6.93 (td, 1H), 6.38 (s, 1H), 5.27 (d, J = 10.8 Hz, 1H), 4.06-4.03 (m, 1H), 3.96 (s, 3H), 3.61 (s, 3H), 2.11 (s, 3H), 1.29 (d, J = 6.5 Hz, 3H). D1E2 ¹H NMR (400 MHz, DMSO- d₆) δ 11.17 (brs, 1H), 10.53 (s, 1H), 9.33 (s, 1H), 8.93 (s, 1H), 7.63 (dd, 1H), 7.28 (dd, | Isomer- 1_(D1E1)_LCMS: m/ [symbol] 501.1 [M⁺ + 1]. Isomer- 2_(D1E2)_LCMS: m/ [symbol] 501.1 [M⁺ + 1]. Isomer- 3_(D2E1)_LCMS: m/ [symbol] 501.1 [M⁺ + 1]. Isomer- 4_(D2E2)_LCMS: m/ [symbol] 501.1 [M⁺ + 1]. |
| | | 1H), 6.93 (td, 1H), 6.38 (s, | |
| | | 1H), 5.27 (d, J = 10.8 Hz, | |
| | | 1H), 4.06-4.03 (m, 1H), 3.96 | |
| | | (s, 3H), 3.61 (s, 3H), 2.11 (s, | |
| | | 3H), 1.29 (d, J = 6.5 Hz, 3H). | |
| | | D2E1 | |
| | | ¹H NMR (400 MHz, DMSO- | |
| | | d₆) δ 11.22 (brs, 1H), 10.56 | |
| | | (s, 1H), 9.25 (s, 1H), 8.81 (s, | |
| | | 1H), 7.85 − 7.63 (m, 1H), | |
| | | 7.48 (dd, 1H), 7.12 (td, 1H), | |
| | | 6.07 (s, 1H), 5.16 (d, J = 10.9 | |
| | | Hz, 1H), 3.94-3.89 (m, 1H), | |
| | | 3.58 (s, 6H), 1.89 (s, 3H), | |
| | | 1.11 (d, J = 6.6 Hz, 3H). | |
| | | D2E2 | |
| | | ¹H NMR (400 MHz, DMSO- | |
| | | d₆) δ 11.29 (s, 1H), 10.63 (s, | |
| | | 1H), 9.32 (s, 1H), 8.88 (s, | |
| | | 1H), 7.78 (dd, 1H), 7.55 (dd, | |
| | | 1H), 7.20 (td, 1H), 6.14 (s, | |
| | | 1H), 5.23 (d, J = 10.9 Hz, | |
| | | 1H), 3.99-3.89 (m, 1H), 3.65 | |
| | | (s, 6H), 1.96 (s, 3H), 1.18 (d, | |
| | | J = 6.6 Hz, 3H). | |
| Example 114 | 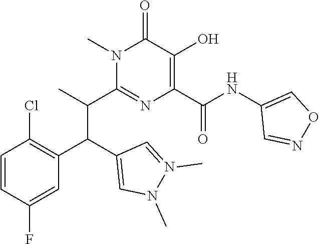 | D1E1 ¹H NMR (400 MHz, DMSO- d₆) δ 11.20 (s, 1H), 10.41 (s, 1H), 9.33 (s, 1H), 8.97 (s, 1H), 7.54 (d, J = 10.1 Hz, 1H), 7.24 (t, J = 6.7 Hz, 1H), 6.88 (d, J = 8.7 Hz, 1H), 6.24 (s, 1H), 5.17 (d, J = 11.1 Hz, 1H), 4.06-4.01 (m, 1H), 3.67 (s, 3H), 3.60(s, 3H), 2.21 (s, 3H), 1.29 (d, J = 6.5 Hz, 3H). D1E2 ¹H NMR (400 MHz, DMSO- d₆) δ 11.19 (s, 1H), 10.42 (s, 1H), 9.33 (s, 1H), 8.97 (s, | Isomer- 1_(D1E1)_LCMS: m/ [symbol] 501.1 [M⁺ + 1]. Isomer- 2_(D1E2)_LCMS: m/ [symbol] 501.1 [M⁺ + 1]. Isomer- 3_(D2E1)_LCMS: m/ [symbol] 501.1 [M⁺ + 1]. Isomer- 4_(D2E2)_LCMS: m/ [symbol] 501.1 [M⁺ + 1]. |
| | | 1H), 7.54 (d, J = 10.1 Hz, | |
| | | 1H), 7.24 (t, J = 6.7 Hz, 1H), | |
| | | 6.88 (d, J = 8.7 Hz, 1H), 6.24 | |
| | | (s, 1H), 5.17 (d, J = 11.1 Hz, | |
| | | 1H), 4.06-4.01 (m, 1H), 3.67 | |
| | | (s, 3H), 3.60(s, 3H), 2.21 (s, | |
| | | 3H), 1.30 (d, 3H). | |
| | | D2E1 | |
| | | ¹H NMR (400 MHz, DMSO- | |
| | | d₆) & 11.18 (brs, 1H), 10.44 | |
| | | (s, 1H), 9.33 (s, 1H), 8.92 (s, | |
| | | 1H), 7.72 − 7.41 (m, 2H), | |
| | | 7.25 − 7.02 (m, 1H), 5.70 (s, | |
| | | 1H), 5.02 (d, J = 10.7 Hz, | |
| | | 1H), 4.06-4.01 (m, 1H), | |
| | | 3.62(s, 3H), 3.49 (s, 3H), | |
| | | 2.05 (s, 3H), 1.33 − 0.91 (m, | |
| | | 3H). | |
| | | D2E2 | |
| | | ¹H NMR (400 MHz, DMSO- | |
| | | d₆) δ 11.18 (brs, 1H), 10.42 | |
| | | (s, 1H), 9.33 (s, 1H), 8.92 (s, | |
| | | 1H), 7.72 − 7.41 (m, 2H), | |
| | | 7.25 − 7.02 (m, 1H), 5.70 (s, | |
| | | 1H), 5.02 (d, J = 10.7 Hz, | |
| | | 1H), 4.06-4.01 (m, 1H), | |
| | | 3.62(s, 3H), 3.49 (s, 3H), | |
| | | 2.05 (s, 3H), 1.16 (d, 3H). | |
| Example 115 | 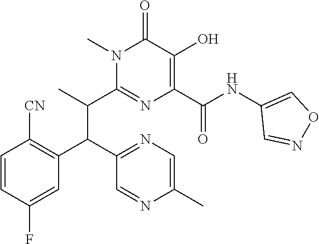 [SMILES: Cc1cnc(C(c2cc(F)ccc2C#N)C(C)c2nc(C(=O)Nc3cnoc3)c(O)c(=O)n2C)cn1] | D1E1 ¹H NMR (400 MHz, Chloroform-d) 8 11.81 (s, 1H), 9.58 (s, 1H), 9.16 (s, 1H), 8.84 (s, 1H), 8.74 (s, 1H), 8.55 (s, 1H), 7.56 − 7.49 (m, 2H), 6.99 − 6.94 (m, 1H), 5.31 − 5.28 (m, 1H), 4.37 − 4.31 (m, 1H), 3.74 (s, 3H), Isomer- 2.62 (s, 3H), 1.21 (d, 3H). 4_(D2E2)_LCMS: D1E2 ¹H NMR (400 MHz, Chloroform-d) δ 11.81 (s, | Isomer- 1_(D1E1)_LCMS: m/ [symbol] 490.1 [M⁺ + 1]. Isomer- 2_(D1E2)_LCMS: m/ [symbol] 490.1 [M⁺ + 1]. Isomer- 3_(D2E1)_LCMS: m/ [symbol] 490.1 [M⁺ + 1]. |
| | | 1H), 9.57 (s, 1H), 9.16 (s, | |
| | | 1H), 8.83 (s, 1H), 8.72 (s, | |
| | | 1H), 8.53 (s, 1H), 7.56 − 7.48 | |
| | | (m, 2H), 6.98 − 6.93 (m, 1H), | |
| | | 5.29 − 5.26 (m, 1H), 4.35 − | |
| | | 4.31 (m, 1H), 3.74 (s, 3H), | |
| | | 2.61 (s, 3H), 1.21 (d, 3H). | |
| | | D2E1 | |
| | | ¹H NMR (400 MHz, DMSO- | |
| | | d₆) δ 11.10 (s, 1H), 10.42 (s, | |
| | | 1H), 9.31 (s, 1H), 8.86 (s, | |
| | | 1H), 8.39 − 8.38 (m, 2H), | |
| | | 8.02 − 7.98 (m, 1H), 7.87 − | |
| | | 7.84 (m, 1H), 7.42 − 7.37 (m, | |
| | | 1H), 5.34 (d, 1H), 4.39 − | |
| | | 4.35 (m, 1H), 3.64 (s, 3H), | |
| | | 2.35 (s, 3H), 1.22 (d, 3H). | |
| | | D2E2 | |
| | | ¹H NMR (400 MHz, DMSO- | |
| | | d₆) δ 11.10 (s, 1H), 10.41 (s, | |
| | | 1H), 9.31 (s, 1H), 8.86 (s, | |
| | | 1H), 8.39 (q, 2H), 8.01 − | |
| | | 7.98 (m, 1H), 7.87 − 7.84 (m, | |
| | | 1H), 7.42 − 7.37 (m, 1H), | |
| | | 5.34 (d, 1H), 4.39 − 4.35 (m, | |
| | | 1H), 3.64 (s, 3H), 2.35 (s, | |
| | | 3H), 1.22 (d, 3H). | |
| Example 116 | 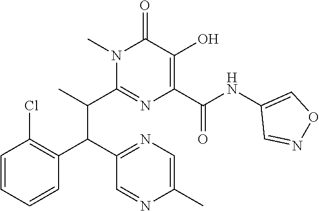 [SMILES: Cc1cnc(C(c2ccccc2Cl)C(C)c2nc(C(=O)Nc3cnoc3)c(O)c(=O)n2C)cn1] | D1E1 ¹H NMR (300 MHz, DMSO- d₆) δ 11.13 (s, 1H), 10.50 (s, 1H), 9.35 (s, 1H), 8.98 (s, 1H), 8.74(s, 1H), 8.58 (s, 1H), 7.69 (d, J = 7.8 Hz, 1H), 7.24 − 7.17 (m, 2H), 7.07- 7.02(m, 1H), 5.55 (d, J = 11.0 Hz, 1H), 4.35-4.29 (m, 1H), 3.63 (s, 3H), 1.21 (d, J = 6.4 Hz, 3H). D1E2 1H NMR (300 MHz, DMSO- | Isomer- 1_(D1E1)_LCMS: m/ [symbol] 481.1 [M⁺ + 1]. Isomer- 2_(D1E2)_LCMS: m/ [symbol] 481.1 [M⁺ + 1]. Isomer- 3_(D2E1)_LCMS: m/ [symbol] 481.1 [M⁺ + 1]. Isomer- 4_(D2E2)_LCMS: m/ [symbol] 481.1 [M⁺ + 1]. |
| | | ¹H NMR (300 MHz, DMSO- | |
| | | d₆) δ 11.14 (brs, 1H), 10.50 | |
| | | (s, 1H), 9.34 (s, 1H), 8.98 (s, | |
| | | 1H), 8.74(s, 1H), 8.58 (s, | |
| | | 1H), 7.69 (d, J = 7.8 Hz, 1H), | |
| | | 7.24 − 7.17 (m, 2H), 7.07- | |
| | | 7.02(m, 1H), 5.55 (d, J = | |
| | | 11.0 Hz, 1H), 4.35-4.29 (m, | |
| | | 1H), 3.63 (s, 3H), 1.22 (d, J = | |
| | | 6.4 Hz, 3H). | |
| | | D2E1 | |
| | | ¹H NMR (300 MHz, DMSO- | |
| | | d₆) δ 11.31 (brs, 1H), 10.51 | |
| | | (s, 1H), 9.31 (s, 1H), 8.93 (s, | |
| | | 1H), 8.35-8.30 (m, 2H), 7.80 | |
| | | (d, J = 7.7 Hz, 1H), 7.61 − | |
| | | 7.18 (m, 3H), 5.45 (d, J = | |
| | | 10.8 Hz, 1H), 4.46 − 4.12 (m, | |
| | | 1H), 3.68 (s, 3H), 2.29 (s, | |
| | | 3H), 1.22 (d, J = 6.6 Hz, 3H). | |
| | | D2E2 | |
| | | ¹H NMR (300 MHz, DMSO- | |
| | | d₆) δ 11.30 (brs, 1H), 10.49 | |
| | | (s, 1H), 9.31 (s, 1H), 8.93 (s, | |
| | | 1H), 8.35-8.30 (m, 2H), 7.80 | |
| | | (d, J = 7.7 Hz, 1H), 7.61 − | |
| | | 7.18 (m, 3H), 5.45 (d, J = | |
| | | 10.8 Hz, 1H), 4.46 − 4.12 (m, | |
| | | 1H), 3.68 (s, 3H), 2.29 (s, | |
| | | 3H), 1.21 (d, J = 6.6 Hz, 3H). | |
| Example 117 | 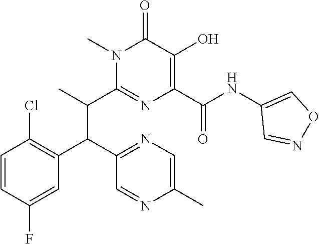 [SMILES: Cc1cnc(C(c2cc(F)ccc2Cl)C(C)c2nc(C(=O)Nc3cnoc3)c(O)c(=O)n2C)cn1] | D1E1 ¹H NMR (400 MHz, Chloroform-d) δ 11.43 (s, 1H), 9.48 (s, 1H), 9.12 (s, 1H), 8.90 (s, 1H), 8.72 (s, 1H), 8.60 (s, 1H), 7.26 − 7.22 (m, 2H), 6.83 − 6.78 (m, 1H), 5.47 (d, 1H), 4.33 − 4.28 (m, 1H), 3.76 (s, 3H), 2.65 (s, 3H), 1.22 (d, 3H). D1E2 ¹H NMR (400 MHz, Chloroform-d) δ 11.42 (s, 1H), 9.38 (s, 1H), 9.11 (s, 1H), 8.79 (s, 1H), 8.68 (s, 1H), 8.54 (s, 1H), 7.26 − 7.21 | Isomer- 1_(D1E1)_LCMS: m/ [symbol] 499.1 [M⁺ + 1]. Isomer- 2_(D1E2)_LCMS: m/ [symbol] 499.1 [M⁺ + 1]. Isomer- 3_(D2E1)_LCMS: m/ [symbol] 499.1 [M⁺ + 1]. Isomer- 4_(D2E2)_LCMS: m/ [symbol] 499.1 [M⁺ + 1]. |
| | | (m, 2H), 6.82 − 6.77 (m, 1H), | |
| | | 5.35 (d, 1H), 4.32 (s, 1H), | |
| | | 3.77 (s, 3H), 2.62 (s, 3H), | |
| | | 1.21 (d, 3H). | |
| | | D2E1 | |
| | | ¹H NMR (400 MHz, DMSO- | |
| | | d₆) δ 11.29 (s, 1H), 10.50 (s, | |
| | | 1H), 9.32 (s, 1H), 8.93 (s, | |
| | | 1H), 8.37 − 8.35 (m, 2H), | |
| | | 7.78 − 7.74 (m, 1H), 7.55 − | |
| | | 7.51 (m, 1H), 7.22 − 7.17 (m, | |
| | | 1H), 5.39 (d, 1H), 4.30 − | |
| | | 4.26 (m, 1H), 3.65 (s, 3H), | |
| | | 2.32 (s, 3H), 1.24 (d, 3H). | |
| | | D2E2 | |
| | | ¹H NMR (400 MHz, DMSO- | |
| | | d₆) δ 11.28 (s, 1H), 10.51 (s, | |
| | | 1H), 9.32 (s, 1H), 8.93 (s, | |
| | | 1H), 8.38 − 8.34 (m, 2H), | |
| | | 7.78 − 7.74 (m, 1H), 7.55 − | |
| | | 7.51 (m, 1H), 7.22 − 7.17 (m, | |
| | | 1H), 5.39 (d, 1H), 4.32 − | |
| | | 4.24 (m, 1H), 3.65 (s, 3H), | |
| | | 2.32 (s, 3H), 1.24 (d, 3H). | |
| Example 118 | 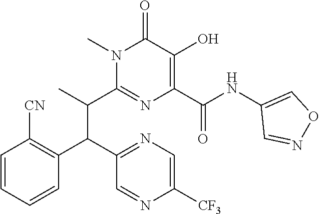 [SMILES: CC(c1nc(C(=O)Nc2cnoc2)c(O)c(=O)n1C)C(c1cnc(C(F)(F)F)cn1)c1ccccc1C#N] | D1E1 ¹H NMR (400 MHz, DMSO- d₆) δ 11.20 (s, 1H), 10.58 (s, 1H), 9.32 (s, 1H), 9.26 (s, 1H), 9.16 (s, 1H), 8.89 (s, 1H), 7.90 (d, 1H), 7.68 − 7.59 (m, 2H), 7.30 (t, 1H), 5.60 (d, 1H), 4.46 − 4.42 (m, 1H), 3.62 (s, 3H), 1.29 (d, 3H). D1E2 ¹H NMR (400 MHz, DMSO- d₆) δ 11.20 (s, 1H), 10.58 (s, 1H), 9.31 (s, 1H), 9.26 (s, | Isomer- 1_(D1E1)_LCMS: m/ [symbol] 526.2 [M⁺ + 1]. Isomer- 2_(D1E2)_LCMS: m/ [symbol] 526.2 [M⁺ + 1]. Isomer- 3_(D2E1)_LCMS: m/ [symbol] 526.2 [M⁺ + 1]. Isomer- 4_(D2E2)_LCMS: m/ [symbol] 526.2 [M⁺ + 1]. |
| | | 1H), 9.16 (s, 1H), 8.89 (s, | |
| | | 1H), 7.90 (d, 1H), 7.68 − | |
| | | 7.59 (m, 2H), 7.30 (t, 1H), | |
| | | 5.60 (d, 1H), 4.46 − 4.42 (m, | |
| | | 1H), 3.62 (s, 3H), 3.30 (s, | |
| | | 1H), 1.29 (d, 3H). | |
| | | D2E1 | |
| | | ¹H NMR (400 MHz, DMSO- | |
| | | d₆) δ 11.14 (s, 1H), 10.33 (s, | |
| | | 1H), 9.29 (s, 1H), 9.05 (s, | |
| | | 1H), 8.83 (s, 1H), 8.77 (s, | |
| | | 1H), 7.93 (d, 1H), 7.85 (d, | |
| | | 1H), 7.78 (t, 1H), 7.55 (t, | |
| | | 1H), 5.62 (d, 1H), 4.39 − | |
| | | 4.31 (m, 1H), 3.69 (s, 3H), | |
| | | 1.19 (d, 3H). | |
| | | D2E2 | |
| | | ¹H NMR (400 MHz, DMSO- | |
| | | d₆) δ 11.14 (s, 1H), 10.32 (s, | |
| | | 1H), 9.29 (s, 1H), 9.05 (s, | |
| | | 1H), 8.83 (s, 1H), 8.77 (s, | |
| | | 1H), 7.93 (d, 1H), 7.85 − | |
| | | 7.76 (m, 2H), 7.55 (t, 1H), | |
| | | 5.61 (d, 1H), 4.37 − 4.33 (m, | |
| | | 1H), 3.69 (s, 3H), 1.18 (d, | |
| | | 3H). | |
| Example 119 | 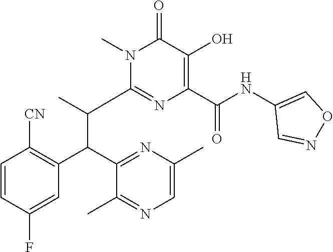 [SMILES: Cc1cnc(C)c(C(c2cc(F)ccc2C#N)C(C)c2nc(C(=O)Nc3cnoc3)c(O)c(=O)n2C)n1] | D1E1 ¹H NMR (300 MHz, DMSO- d₆) δ 11.32 (brs, 1H), 10.54 (s, 1H), 9.31 (s, 1H), 8.85 (s, 1H), 8.37 (s, 1H), 7.76-7.62 (m, 2H), 7.18 (t, 1H), 5.37 (d, 1H), 4.34-4.26 (m, 1H), 3.53 (s, 3H), 2.77 (s, 3H), 2.57 (s, 3H), 1.26 (d, 3H). D1E2 ¹H NMR (400 MHz, DMSO- Isomer- d₆) δ 11.32 (brs, 1H), 10.51 (s, 1H), 9.31 (s, 1H), 8.85 (s, 1H), 8.37 (s, 1H), 7.76 (d, 1H), 7.69 (d, 1H), 7.18 (t, | Isomer- 1_(D1E1)_LCMS: m/ [symbol] 504.1 [M⁺ + 1]. Isomer- 2_(D1E2)_LCMS: m/ [symbol] 504.1 [M⁺ + 1]. Isomer- 3_(D2E1)_LCMS: m/ [symbol] 504.1 [M⁺ + 1]. Isomer- 4_(D2E2)_LCMS: m/ [symbol] 504.1 [M⁺ + 1]. |
| | | 1H), 5.37 (d, 1H), 4.34-4.27 | |
| | | (m, 1H), 3.54 (s, 3H), 2.78 | |
| | | (s, 3H), 2.57 (s, 3H), 1.26 (d, | |
| | | 3H). | |
| | | D2E1 | |
| | | ¹H NMR (300 MHz, DMSO- | |
| | | d₆) δ 11.11 (brs, 1H), 10.49 | |
| | | (s, 1H), 9.35 (s, 1H), 8.87 (s, | |
| | | 1H), 8.23 (s, 1H), 8.15 − 8.07 | |
| | | (m, 1H), 7.62 (dd, 1H), 7.54 − | |
| | | 7.43 (m, 1H), 5.43 (d, 1H), | |
| | | 4.36-4.27 (m, 1H), 3.81 (s, | |
| | | 3H), 2.49 (s, 3H), 2.43 (s, | |
| | | 3H), 1.28 (d, 3H). | |
| | | D2E2 | |
| | | ¹H NMR (300 MHz, DMSO- | |
| | | d₆) δ 11.11 (brs, 1H), 10.48 | |
| | | (s, 1H), 9.35 (s, 1H), 8.87 (s, | |
| | | 1H), 8.23 (s, 1H), 8.15 − 8.07 | |
| | | (m, 1H), 7.62 (dd, 1H), 7.54 − | |
| | | 7.43 (m, 1H), 5.43 (d, 1H), | |
| | | 4.36-4.27 (m, 1H), 3.81 (s, | |
| | | 3H), 2.49 (s, 3H), 2.43 (s, | |
| | | 3H), 1.28 (d, 3H). | |
| Example 120 | 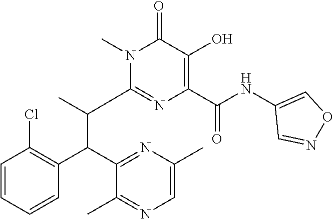 [SMILES: Cc1cnc(C)c(C(c2ccccc2Cl)C(C)c2nc(C(=O)Nc3cnoc3)c(O)c(=O)n2C)n1] | D1E1 ¹H NMR (300 MHz, DMSO- d₆) δ 11.26 (s, 1H), 10.56 (s, 1H), 9.35 (s, 1H), 8.97 (s, 1H), 8.32 (s, 1H), 7.66 (d, 1H), 7.29 − 7.13 (m, 2H), 7.07 (t, 1H), 5.41 (d, 1H), 4.36-4.25 (m, 1H), 3.45 (s, 3H), 2.70 (s, 3H), 2.55 (s, 3H), 1.30 (d, 3H). D1E2 ¹H NMR (300 MHz, DMSO- d₆) δ 11.26 (s, 1H), 10.55 (s, 1H), 9.35 (s, 1H), 8.97 (s, | Isomer- 1_(D1E1)_LCMS: m/ [symbol] 495.1 [M⁺ + 1]. Isomer- 2_(D1E2)_LCMS: m/ [symbol] 495.1 [M⁺ + 1]. Isomer- 3_(D2E1)_LCMS: m/ [symbol] 495.1 [M⁺ + 1]. Isomer- 4_(D2E2)_LCMS: m/ [symbol] 495.1 [M⁺ + 1]. |
| | | 1H), 8.32 (s, 1H), 7.66 (d, | |
| | | 1H), 7.27 − 7.14 (m, 2H), | |
| | | 7.07 (t, 1H), 5.41 (d, 1H), | |
| | | 4.36-4.26 (m, 3.45 (s, 3H), | |
| | | 2.70 (s, 3H), 2.55 (s, 3H), | |
| | | 1.30 (d, 3H). | |
| | | D2E1 | |
| | | ¹H NMR (400 MHz, DMSO- | |
| | | d₆) δ 11.17 (s, 1H), 10.43 (s, | |
| | | 1H), 9.27 (s, 1H), 8.85 (s, | |
| | | 1H), 8.08 (s, 1H), 7.57 − 7.42 | |
| | | (m, 2H), 7.35-7.28 (m, 2H), | |
| | | 5.52 (d, 1H), 4.04-3.99 (m, | |
| | | 1H), 3.77 (s, 3H), 2.37 (s, | |
| | | 3H), 2.30 (s, 3H), 1.23 (d, | |
| | | 3H). | |
| | | D2E2 | |
| | | ¹H NMR (400 MHz, DMSO- | |
| | | d₆) δ 11.16 (s, 1H), 10.43 (s, | |
| | | 1H), 9.27 (s, 1H), 8.84 (s, | |
| | | 1H), 8.09 (s, 1H), 7.59 − 7.41 | |
| | | (m, 2H), 7.35-7.28 (m, 2H), | |
| | | 5.50 (d, 1H), 4.04-3.99 (m, | |
| | | 1H), 3.77 (s, 3H), 2.36 (s, | |
| | | 3H), 2.30 (s, 3H), 1.24 (d, | |
| | | 3H). | |
| Example 121 | 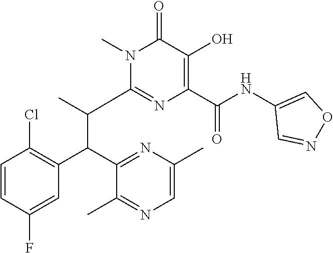 [SMILES: Cc1cnc(C)c(C(c2cc(F)ccc2Cl)C(C)c2nc(C(=O)Nc3cnoc3)c(O)c(=O)n2C)n1] | D1E1 ¹H NMR (400 MHz, DMSO- d₆) δ 11.25 (s, 1H), 10.51 (s, 1H), 9.35 (s, 1H), 8.96 (s, 1H), 8.34 (s, 1H), 7.50 (dd, 1H), 7.30 (dd, 1H), 7.00-6.95 (m, 1H), 5.45 (d, 1H), 4.25- 4.21 (m, 1H), 3.52 (s, 3H), 2.75 (s, 3H), 2.55 (s, 3H), 1.27 (d, 3H). D1E2 ¹H NMR (400 MHz, DMSO- d₆) δ 11.25 (s, 1H), 10.51 (s, 1H), 9.35 (s, 1H), 8.95 (s, 1H), 8.34 (s, 1H), 7.50 (dd, 1H), 7.30 (dd, 1H), 7.00-6.95 | Isomer- 1_(D1E1)_LCMS: m/ [symbol] 513.0 [M⁺ + 1]. Isomer- 2_(D1E2)_LCMS: m/ [symbol] 513.0 [M⁺ + 1]. Isomer- 3_(D2E1)_LCMS: m/ [symbol] 513.0 [M⁺ + 1]. Isomer- 4_(D2E2)_LCMS: m/ [symbol] 513.0 [M⁺ + 1]. |
| | | (m, 1H), 5.45 (d, 1H), 4.25- | |
| | | 4.21 (m, 1H), 3.52 (s, 3H), | |
| | | 2.75 (s, 3H), 2.55 (s, 3H), | |
| | | 1.26 (d, 3H). | |
| | | D2E1 | |
| | | ¹H NMR (400 MHz, DMSO- | |
| | | d₆) δ 11.18 (s, 1H), 10.46 (s, | |
| | | 1H), 9.28 (s, 1H), 8.86 (s, | |
| | | 1H), 8.12 (s, J = 15.8 Hz, | |
| | | 1H), 7.61 (dd, 1H), 7.34 (s, | |
| | | 1H), 7.23-7.18 (m, 1H), 5.50 | |
| | | (d, 1H), 4.11-4.05 (m, 1H), | |
| | | 3.76 (s, 3H), 2.41 (s, 3H), | |
| | | 2.33 (s, 3H), 1.24 (d, 3H). | |
| | | D2E2 | |
| | | ¹H NMR (400 MHz, DMSO- | |
| | | d₆) δ 11.18 (s, 1H), 10.46 (s, | |
| | | 1H), 9.28 (s, 1H), 8.86 (s, | |
| | | 1H), 8.11 (s, 1H), 7.61 (dd, | |
| | | 1H), 7.34 (s, 1H), 7.23-7.18 | |
| | | (m, 1H), 5.50 (d, 1H), 4.11- | |
| | | 4.05 (m, 1H), 3.76 (s, 3H), | |
| | | 2.41 (s, 3H), 2.33 (s, 3H), | |
| | | 1.24 (d, 3H). | |
| Example 122 | 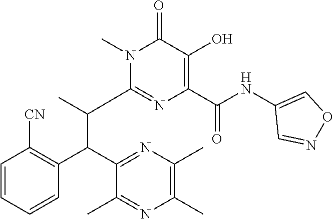 [SMILES: Cc1nc(C)c(C(c2ccccc2C#N)C(C)c2nc(C(=O)Nc3cnoc3)c(O)c(=O)n2C)nc1C] | D1E1 ¹H NMR (300 MHz, DMSO- d₆) δ 11.33 (s, 1H), 10.53 (s, 1H), 9.32 (s, 1H), 8.88 (s, 1H), 7.84 (d, J = 8.0 Hz, 1H), 7.65 − 7.51 (m, 2H), 7.26 (t, 1H), 5.26 (d, 1H), 4.41-4.35 (m, 1H), 3.50 (s, 3H), 2.70 (s, 3H), 2.56 (s, 3H), 2.44 (s, 3H), 1.29 (d, J = 6.5 Hz, 3H). D1E2 ¹H NMR (300 MHz, DMSO- d₆) δ 11.33 (s, 1H), 10.53 (s, 1H), 9.32 (s, 1H), 8.88 (s, | Isomer- 1_(D1E1)_LCMS: m/ [symbol] 500.2 [M⁺ + 1]. Isomer- 2_(D1E2)_LCMS: m/ [symbol] 500.2 [M⁺ + 1]. Isomer- 3_(D2E1)_LCMS: m/ [symbol] 500.2 [M⁺ + 1]. Isomer- 4_(D2E2)_LCMS: m/ [symbol] 500.2 [M⁺ + 1]. |
| | | 1H), 7.84 (d, J = 8.0 Hz, 1H), | |
| | | 7.65 − 7.51 (m, 2H), 7.26 (t, | |
| | | 1H), 5.26 (d, 1H), 4.46-4.33 | |
| | | (m, 1H), 3.50 (s, 3H), 2.70 | |
| | | (s, 3H), 2.56 (s, 3H), 2.44 (s, | |
| | | 3H), 1.29 (d, J = 6.5 Hz, 3H). | |
| | | D2E1 | |
| | | ¹H NMR (300 MHz, DMSO- | |
| | | d₆) δ 11.01 (s, 1H), 10.45 (s, | |
| | | 1H), 9.28 (s, 1H), 8.80 (s, | |
| | | 1H), 7.91 (d, J = 7.7 Hz, 1H), | |
| | | 7.76 − 7.65 (m, 2H), 7.51 (t, | |
| | | 1H), 5.33 (d, J = 10.6 Hz, | |
| | | 1H), 4.27-4.20(m, 1H), 3.76 | |
| | | (s, 3H), 2.34 (s, 6H), 2.29(s, | |
| | | 3H), 1.16 (d, 3H). | |
| | | D2E2 | |
| | | ¹H NMR (300 MHz, DMSO- | |
| | | d₆) δ 11.01 (s, 1H), 10.41 (s, | |
| | | 1H), 9.28 (s, 1H), 8.80 (s, | |
| | | 1H), 7.91 (d, J = 7.7 Hz, 1H), | |
| | | 7.76 − 7.65 (m, 2H), 7.51 (t, | |
| | | 1H), 5.33 (d, J = 10.6 Hz, | |
| | | 1H), 4.27-4.18(m, 1H), 3.76 | |
| | | (s, 3H), 2.34 (s, 6H), 2.29(s, | |
| | | 3H), 1.16 (d, 3H). | |
| Example 123 | 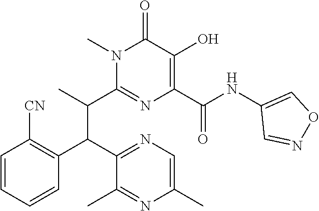 [SMILES: Cc1cnc(C(c2ccccc2C#N)C(C)c2nc(C(=O)Nc3cnoc3)c(O)c(=O)n2C)c(C)n1] | D1E1 ¹H NMR (300 MHz, DMSO- d₆) δ 11.33 (s, 1H), 10.57 (s, 1H), 9.32 (s, 1H), 8.88 (s, 1H), 8.52 (s, 1H), 7.81 (d, 1H), 7.66 − 7.51 (m, 2H), 7.27 (t, 1H), 5.32 (d, 1H), 4.40-4.36 (m, 1H), 3.49 (s, 3H), 2.76 (s, 3H), 2.47 (s, 3H), 1.30 (d, 3H). D1E2 ¹H NMR (300 MHz, DMSO- d₆) δ 11.33 (s, 1H), 10.57 (s, | Isomer- 1_(D1E1)_LCMS: m/ [symbol] 486.1 [M⁺ + 1]. Isomer- 2_(D1E2)_LCMS: m/ [symbol] 486.1 [M⁺ + 1]. Isomer- 3_(D2E1)_LCMS: m/ [symbol] 486.1 [M⁺ + 1]. Isomer- 4_(D2E2)_LCMS: m/ [symbol] 486.1 [M⁺ + 1]. |
| | | 1H), 9.32 (s, 1H), 8.88 (s, | |
| | | 1H), 8.52 (s, 1H), 7.81 (d, | |
| | | 1H), 7.66 − 7.51 (m, 2H), | |
| | | 7.27 (t, 1H), 5.32 (d, 1H), | |
| | | 4.39-4.36 (m, 1H), 3.49 (s, | |
| | | 3H), 2.76 (s, 3H), 2.47 (s, | |
| | | 3H), 1.30 (d, 3H). | |
| | | D2E1 | |
| | | ¹H NMR (300 MHz, DMSO- | |
| | | d₆) δ 11.00 (s, 1H), 10.39 (s, | |
| | | 1H), 9.28 (s, 1H), 8.79 (s, | |
| | | 1H), 8.25 (s, 1H), 7.91 (d, | |
| | | 1H), 7.74 − 7.67 (m, 2H), | |
| | | 7.50 (t, 1H), 5.38 (d, 1H), | |
| | | 4.28-4.23 (m, 1H), 3.70 (s, | |
| | | 3H), 2.44 (s, 3H), 2.31 (s, | |
| | | 3H), 1.17 (d, 3H). | |
| | | D2E1 | |
| | | ¹H NMR (300 MHz, DMSO- | |
| | | d₆) δ 11.00 (s, 1H), 10.39 (s, | |
| | | 1H), 9.28 (s, 1H), 8.79 (s, | |
| | | 1H), 8.25 (s, 1H), 7.91 (d, | |
| | | 1H), 7.74 − 7.67 (m, 2H), | |
| | | 7.50 (t, 1H), 5.38 (d, 1H), | |
| | | 4.27-4.23 (m, 1H), 3.70 (s, | |
| | | 3H), 2.44 (s, 3H), 2.31 (s, | |
| | | 3H), 1.17 (d, 3H). | |
| Example 124 | 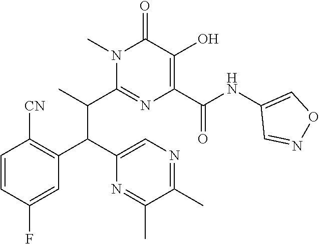 [SMILES: Cc1ncc(C(c2cc(F)ccc2C#N)C(C)c2nc(C(=O)Nc3cnoc3)c(O)c(=O)n2C)nc1C] | D1E1 ¹H NMR (300 MHz, DMSO- d₆) δ 11.22 (s, 1H), 10.51 (s, 1H), 9.31 (s, 1H), 8.89 (s, 1H), 8.62 (s, 1H), 7.84 − 7.70 (m, 2H), 7.14 (t, 1H), 5.35 (d, J = 10.9 Hz, 1H), 4.35- 4.26 (m, 1H), 3.64 (s, 3H), 2.53 (s, 3H), 2.47 (s, 3H), 1.23 (d, J = 6.4 Hz, 3H). D1E2 ¹H NMR (300 MHz, DMSO- d₆) δ 11.22 (s, 1H), 10.51 (s, 1H), 9.31 (s, 1H), 8.89 (s, 1H), 8.62 (s, 1H), 7.84 − 7.70 | Isomer- 1_(D1E1)_LCMS: m/ [symbol] 504.1 [M⁺ + 1]. Isomer- 2_(D1E2)_LCMS: m/ [symbol] 504.1 [M⁺ + 1]. Isomer- 3_(D2E1)_LCMS: m/ [symbol] 504.1 [M⁺ + 1]. Isomer- 4_(D2E2)_LCMS: m/ [symbol] 504.1 [M⁺ + 1]. |
| | | (m, 2H), 7.14 (t, 1H), 5.35 | |
| | | (d, J = 10.9 Hz, 1H), 4.33- | |
| | | 4.24 (m, 1H), 3.64 (s, 3H), | |
| | | 2.53 (s, 3H), 2.47 (s, 3H), | |
| | | 1.22 (d, J = 6.4 Hz, 3H). | |
| | | D2E1 | |
| | | ¹H NMR (300 MHz, DMSO- | |
| | | d₆) δ 11.10 (s, 1H), 10.39 (s, | |
| | | 1H), 9.30 (s, 1H), 8.86 (s, | |
| | | 1H), 8.11 (s, 1H), 8.01 (dd, | |
| | | 1H), 7.79 (dd, 1H), 7.41 (td, | |
| | | 1H), 5.30 (d, J = 10.5 Hz, | |
| | | 1H), 4.38-4.28 (m, 1H), 3.70 | |
| | | (s, 3H), 2.34 (s, 6H), 1.19 (d, | |
| | | J = 6.7 Hz, 3H). | |
| | | D2E2 | |
| | | ¹H NMR (300 MHz, DMSO- | |
| | | d₆) δ 11.10 (s, 1H), 10.40 (s, | |
| | | 1H), 9.30 (s, 1H), 8.86 (s, | |
| | | 1H), 8.11 (s, 1H), 8.01 (dd, | |
| | | 1H), 7.79 (dd, 1H), 7.41 (td, | |
| | | 1H), 5.30 (d, J = 10.5 Hz, | |
| | | 1H), 4.36-4.30 (m, 1H), 3.70 | |
| | | (s, 3H), 2.34 (s, 6H), 1.18 (d, | |
| | | J = 6.7 Hz, 3H). | |
| Example 125 | 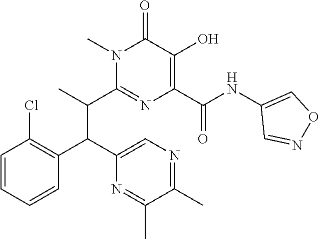 [SMILES: Cc1ncc(C(c2ccccc2Cl)C(C)c2nc(C(=O)Nc3cnoc3)c(O)c(=O)n2C)nc1C] | D1E1 ¹H NMR (400 MHz, Chloroform-d) δ 11.38 (s, 1H), 9.44 (s, 1H), 9.11 (s, 1H), 8.71 (s, 1H), 8.59 (s, 1H), 7.50 (dd, 1H), 7.24 (dd, 1H), 7.16 (td, 1H), 7.05 (td, 1H), 5.35 (d, 1H), 4.43 − 4.34 (m, 1H), 3.77 (s, 3H), 2.60(s, 3H), 2.57 (s, 3H), 1.22 (d, 3H). D1E2 ¹H NMR (400 MHz, methanol-d4) δ 9.23 (s, 1H), 8.83 (s, 1H), 8.49 (s, 1H), | Isomer- 1_(D1E1)_LCMS: m/ [symbol] 495.2 [M⁺ + 1]. Isomer- 2_(D1E2)_LCMS: m/ [symbol] 495.2 [M⁺ + 1]. Isomer- 3_(D2E1)_LCMS: m/ [symbol] 495.2 [M⁺ + 1]. Isomer- 4_(D2E2)_LCMS: m/ [symbol] 495.2 [M⁺ + 1]. |
| | | 7.73 − 7.70 (m, 1H), 7.23 − | |
| | | 7.17 (m, 2H), 7.07 − 7.05 (m, | |
| | | 1H), 5.54 (d, 1H), 4.48-4.43 | |
| | | (m, 1H), 3.74 (s, 3H), 2.59 | |
| | | (s, 3H), 2.52 (s, 3H), 1.26 (d, | |
| | | 3H). | |
| | | D2E1 | |
| | | ¹H NMR (400 MHz, DMSO- | |
| | | d₆) δ 11.27 (s, 1H), 10.43 (s, | |
| | | 1H), 9.30 (s, 1H), 8.92 (s, | |
| | | 1H), 8.05 (s, 1H), 7.73 (s, | |
| | | 1H), 7.50 − 7.47 (m, 1H), | |
| | | 7.40 (d, 1H), 7.31 (t, 1H), | |
| | | 5.39 (d, 1H), 4.22-4.17 (m, | |
| | | 1H), 3.73 (s, 3H), 2.31 (s, | |
| | | 6H), 1.19 (d, 3H). | |
| | | D2E2 | |
| | | ¹H NMR (400 MHz, DMSO- | |
| | | d₆) δ 11.27 (s, 1H), 10.44 (s, | |
| | | 1H), 9.30 (s, 1H), 8.92 (s, | |
| | | 1H), 8.05 (s, 1H), 7.73 (s, | |
| | | 1H), 7.50 − 7.48 (m, 1H), | |
| | | 7.40 (s, 1H), 7.31 (t, 1H), | |
| | | 5.39 (d, 1H), 4.22-4.16 (m, | |
| | | 1H), 3.73 (s, 3H), 2.31 (d, | |
| | | 6H), 1.19 (d, 3H). | |
| Example 126 | 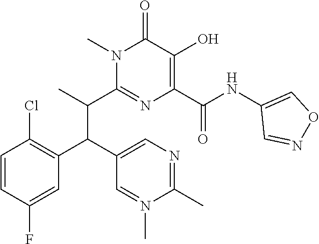 | D1E1 ¹H NMR (400 MHz, DMSO- d₆) δ 11.16 (s, 1H), 10.45 (s, 1H), 9.34 (s, 1H), 8.97 (s, 1H), 8.57 (s, 1H), 7.59 (d, 1H), 7.29 (dd,1H), 6.96-6.91 (m, 1H), 5.47 (d, J = 11.0, 1H), 4.27-4.19 (m, 1H), 3.65 (s, 3H), 2.52 (s, 3H), 2.46 (s, 3H), 1.20 (d, J = 6.6 Hz, 3H). D1E2 ¹H NMR (400 MHz, DMSO- d₆) δ 11.16 (s, 1H), 10.46 (s, 1H), 9.35 (s, 1H), 8.97 (s, 1H), 8.57 (s, 1H), 7.59 (d, | Isomer- 1_(D1E1)_LCMS: m/ [symbol] 513.2 [M⁺ + 1]. Isomer- 2_(D1E2)_LCMS: m/ [symbol] 513.2 [M⁺ + 1]. Isomer- 3_(D2E1)_LCMS: m/ [symbol] 513.2 [M⁺ + 1]. Isomer- 4_(D2E2)_LCMS: m/ [symbol] 513.2 [M⁺ + 1]. |
| | | 1H), 7.29 (dd,1H), 6.96-6.91 | |
| | | (m, 1H), 5.47 (d, J = 11.0, | |
| | | 1H), 4.27-4.19 (m, 1H), 3.65 | |
| | | (s, 3H), 2.52 (s, 3H), 2.46 (s, | |
| | | 3H), 1.20 (d, J = 6.6 Hz, 3H). | |
| | | D2E1 | |
| | | ¹H NMR (400 MHz, DMSO- | |
| | | d₆) δ 11.28 (s, 1H), 10.47 (s, | |
| | | 1H), 9.31 (s, 1H), 8.92 (s, | |
| | | 1H), 8.09 (s, 1H), 7.68 (d, | |
| | | 1H), 7.54 (dd, 1H), 7.25 − | |
| | | 7.16 (m, 1H), 5.35 (d, J = | |
| | | 10.8 Hz, 1H), 4.26-4.21 (m, | |
| | | 1H), 3.71 (s, 3H), 2.33-2.31 | |
| | | (m, 6H), 1.20 (d, 3H). | |
| | | D2E2 | |
| | | ¹H NMR (400 MHz, DMSO- | |
| | | d₆) δ 11.28 (s, 1H), 10.47 (s, | |
| | | 1H), 9.31 (s, 1H), 8.92 (s, | |
| | | 1H), 8.09 (s, 1H), 7.68 (d, | |
| | | 1H), 7.54 (dd, 1H), 7.25 − | |
| | | 7.16 (m, 1H), 5.35 (d, J = | |
| | | 10.8 Hz, 1H), 4.26-4.21 (m, | |
| | | 1H), 3.71 (s, 3H), 2.33-2.31 | |
| | | (m, 6H), 1.20 (d, 3H). | |
| Example 127 | 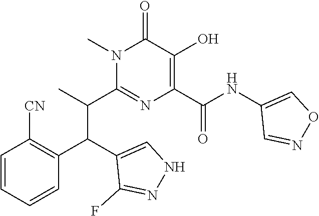 [SMILES: CC(c1nc(C(=O)Nc2cnoc2)c(O)c(=O)n1C)C(c1ccccc1C#N)c1c[nH]nc1F] | D1E1 ¹H NMR (400 MHz, DMSO- d₆) δ 12.44 (s, 1H), 11.37 (brs, 1H), 10.76 (s, 1H), 9.28 (s, 1H), 8.85 (s, 1H), 7.95 (s, 1H), 7.70 − 7.46 (m, 3H), 7.23 (t, 1H), 4.93 (d, 1H), 4.27 − 3.97 (m, 1H), 3.56 (s, 3H), 1.38 (d, 3H). D1E2 ¹H NMR (400 MHz, DMSO- d₆) δ 12.44 (s, 1H), 11.26 (s, 1H), 10.70 (s, 1H), 9.28 (s, 1H), 8.85 (s, 1H), 7.95 (s, | Isomer- 1_(D1E1)_LCMS: m/ [symbol] 464.2 [M⁺ + 1]. Isomer- 2_(D1E2)_LCMS: m/ [symbol] 464.2 [M⁺ + 1]. |
| | | 1H), 7.70 − 7.48 (m, 3H), | |
| | | 7.23 (t, 1H), 4.93 (d, 1H), | |
| | | 4.27 − 3.97 (m, 1H), 3.56 (s, | |
| | | 3H), 1.38 (d, 3H). | |
| Example 128 | 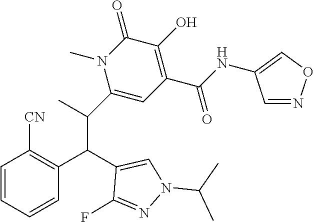 [SMILES: CC(c1cc(C(=O)Nc2cnoc2)c(O)c(=O)n1C)C(c1ccccc1C#N)c1cn(C(C)C)nc1F] | D1E1 ¹H NMR (400 MHz, DMSO- d₆) δ 11.19 (s, 1H), 10.36 (s, 1H), 9.23 (s, 1H), 8.80 (s, 1H), 7.95 (d, 1H), 7.65 − 7.49 (m, 3H), 7.17 (t, 1H), 4.85 (d, 1H), 4.30 − 4.23 (m, 1H), 4.02 − 3.98 (m, 1H), 3.50 (s, 3H), 1.35-1.29 (m, 9H). D1E2 ¹H NMR (400 MHz, DMSO- d₆) δ 11.19 (s, 1H), 10.36 (s, 1H), 9.23 (s, 1H), 8.80 (s, | Isomer- 1_(D1E1)_LCMS: m/ [symbol] 506.3 [M + H]. Isomer- 2_(D1E2)_LCMS: m/ [symbol] 506.3 [M + H]. |
| | | 1H), 7.95 (d, 1H), 7.65 (d, | |
| | | 1H), 7.54 − 7.49 (m, 2H), | |
| | | 7.17 (t, 1H), 4.85 (d, 1H), | |
| | | 4.30 − 4.23 (m, 1H), 4.02 − | |
| | | 3.98 (m, 1H), 3.50 (s, 3H), | |
| | | 1.35-1.29 (m, 9H). | |
| Example 129 | 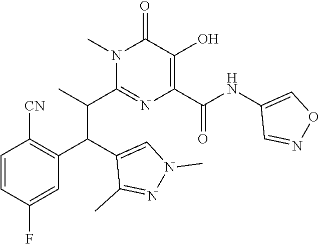 | D1E1 ¹H NMR (400 MHz, DMSO- d₆) δ 11.25 (s, 1H), 10.45 (s, 1H), 9.29 (s, 1H), 8.85 (s, 1H), 7.92 (d, 1H), 7.71 − 7.66 (m, 2H), 7.13 (t, 1H), 4.97 (d, 1H), 4.08 − 4.03 (m, 1H), 3.73 (s, 3H), 3.61 (s, 3H), 1.38 (d, 3H). D1E2 ¹H NMR (400 MHz, DMSO- d₆) δ 11.25 (s, 1H), 10.41 (s, 1H), 9.29 (s, 1H), 8.85 (s, 1H), 7.92 (d, 1H), 7.71 − 7.66 (m, 2H), 7.13 (t, 1H), 4.97 (d, 1H), 4.08 − 4.03 (m, | Isomer- 1_(D1E1)_LCMS: m/ [symbol] 496.0 [M⁺ + 1]. Isomer- 2_(D1E2)_LCMS: m/ [symbol] 496.0 [M⁺ + 1]. |
| | | 1H), 3.73 (s, 3H), 3.61 (s, | |
| | | 3H), 1.39 (d, 3H). | |
| Example 130 | 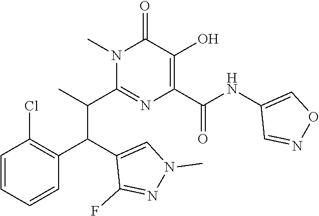 [SMILES: CC(c1nc(C(=O)Nc2cnoc2)c(O)c(=O)n1C)C(c1ccccc1Cl)c1cn(C)nc1F] | D1E1 ¹H NMR (300 MHz, DMSO- d₆) δ 11.18 (s, 1H), 10.38 (s, 1H), 9.32 (s, 1H), 8.95 (s, 1H), 7.76 (d, 1H), 7.55 (d, 1H), 7.24 − 7.18 (m, 2H), 7.08 − 7.02 (m, 1H), 5.07 (d, 1H), 4.03 − 3.97 (m, 1H), 3.70 (d, 3H), 3.60 (s, 3H), 1.37 (d, 3H). D1E2 ¹H NMR (300 MHz, DMSO- d₆) δ 11.18 (s, 1H), 10.37 (s, 1H), 9.32 (s, 1H), 8.95 (s, | Isomer- 1_(D1E1)_LCMS: m/ [symbol] 487.0 [M⁺ + 1]. Isomer- 2_(D1E2)_LCMS: m/ [symbol] 487.0 [M⁺ + 1]. Isomer- 3_(D2E1)_LCMS: m/ z 487.2 [M⁺ + 1]. Isomer- 4_(D2E2)_LCMS: m/ z 487.2 [M⁺ + 1]. |
| | | 1H), 7.76 (d, 1H), 7.55 (d, | |
| | | 1H), 7.24 − 7.18 (m, 2H), | |
| | | 7.08 − 7.02 (m, 1H), 5.07 (d, | |
| | | 1H), 4.03 − 3.97 (m, 1H), | |
| | | 3.70 (d, 3H), 3.60 (s, 3H), | |
| | | 1.37 (d, 3H). | |
| | | D2E1 | |
| | | ¹H NMR (300 MHz, DMSO- | |
| | | d₆) δ 11.33 (s, 1H), 10.43 (s, | |
| | | 1H), 9.34 (s, 1H), 8.92 (s, | |
| | | 1H), 7.83 (d, 1H), 7.47 − | |
| | | 7.41 (m, 3H), 7.31 (td, 1H), | |
| | | 4.90 (d, 1H), 4.03-3.98 (m, | |
| | | 1H), 3.54 (s, 6H), 1.21 (d, | |
| | | 3H) | |
| | | D2E2 | |
| | | ¹H NMR (300 MHz, DMSO- | |
| | | d₆) δ 11.33 (s, 1H), 10.43 (s, | |
| | | 1H), 9.34 (s, 1H), 8.92 (s, | |
| | | 1H), 7.83 (d, 1H), 7.47 − | |
| | | 7.41 (m, 3H), 7.31 (td, 1H), | |
| | | 4.90 (d, 1H), 4.03-3.98 (m, | |
| | | 1H), 3.54 (s, 6H), 1.21 (d, | |
| | | 3H). | |
| Example 154 | 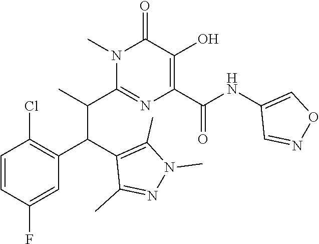 [SMILES: Cc1nn(C)c(C)c1C(c1cc(F)ccc1Cl)C(C)c1nc(C(=O)Nc2cnoc2)c(O)c(=O)n1C] | | Isomer-1_D1E1: m/ [symbol] 515.1 [M + H] >98% ee Isomer-2_D1E2: m/ [symbol] 515.1 [M + H] >98% ee |

### Example 31

### Synthesis of 2-(1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide

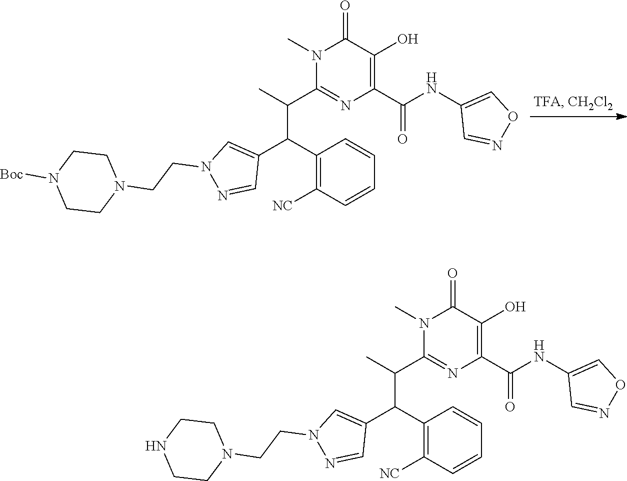 [SMILES: CC(c1nc(C(=O)Nc2cnoc2)c(O)c(=O)n1C)C(c1cnn(CCN2CCN(C(=O)OC(C)(C)C)CC2)c1)c1ccccc1C#N ; CC(c1nc(C(=O)Nc2cnoc2)c(O)c(=O)n1C)C(c1cnn(CCN2CCNCC2)c1)c1ccccc1C#N]

Step 1: (2-(1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide). To a 0° C. stirred solution of tert-butyl 4-(2-(4-(1-(2-cyanophenyl)-2-(5-hydroxy-4-(isoxazol-4-ylcarbamoyl)-1-methyl-6-oxo-1,6-dihydropyrimidin-2-yl)propyl)-1H-pyrazol-1-yl)ethyl)piperazine-1-carboxylate (0.102 g, 0.2 mmol) in DCM (4 mL) was added TFA (2 mL) dropwise. The resulting solution was warmed to rt and stirred for 2 h at which point it was concentrated in vacuo. The resulting crude material was purified by reverse phase chromatography.

Isomer-1_D1E1: Isolated a yellow solid (0.069 g, 80% yield)

ESI-MS m/ [symbol] : 558.3 [M+H]⁺; >98% ee

¹H NMR (400 MHz, methanol-d4): δ 9.20 (s, 1H), 8.78 (s, 1H), 8.53 (s, 0.42H) 7.83 (s, 1H), 7.74 (d, J=8.0 Hz, 1H), 7.63 (s, 1H), 7.58-7.46 (m, 2H), 7.20 (t, J=7.7 Hz, 1H), 5.17 (d, J=11.1 Hz, 1H), 4.25 (t, J=6.1 Hz, 2H), 4.08-4.03 (m, 1H), 3.70 (s, 3H), 3.04 (s, 4H), 2.80 (t, J=6.1 Hz, 2H), 2.59 (s, 4H), 1.36 (d, J=6.6 Hz, 3H).

Isomer-2_D1E2: Isolated a yellow solid (0.075 g, 88% yield)

ESI-MS m/ [symbol] : 558.3 [M+H]⁺; >98% ee

¹H NMR (400 MHz, methanol-d4): δ 9.19 (s, 1H), 8.78 (s, 1H), 8.53 (s, 1H), 7.83 (s, 1H), 7.73 (d, J=8.0 Hz, 1H), 7.63 (s, 1H), 7.57-7.48 (m, 2H), 7.18 (t, J=7.4 Hz, 1H), 5.16 (d, J=11.1 Hz, 1H), 4.25 (t, J=6.1 Hz, 2H), 4.09-4.03 (m, 1H), 3.70 (s, 3H), 3.04 (s, 4H), 2.80 (t, J=6.1 Hz, 2H), 2.59 (s, 4H), 1.36 (d, J=6.6 Hz, 3H).

Isomer-3_D2E1: Isolated a yellow solid (0.101 g, 91% yield)

ESI-MS m/ [symbol] : 558.3 [M+H]⁺; 98% ee

¹H NMR (400 MHz, DMSO-d6) δ 9.24 (s, 1H), 8.81 (s, 1H), 8.16 (s, 0.58H), 8.03 (d, J=8.0 Hz, 1H), 7.85-7.72 (m, 2H), 7.46 (t, 1H), 7.33 (s, 1H), 7.10 (s, 1H), 4.72 (d, J=10.8 Hz, 1H), 4.12-3.72 (m, 4H), 3.41 (s, 3H), 2.97-2.91 (m, 4H), 2.79-2.76 (m, 1H), 2.65-2.59 (m, 1H), 2.40-2.20 (m, 4H), 1.08 (d, J=6.5 Hz, 3H).

Isomer-4_D2E2: Isolated a yellow solid (0.104 g 91% yield)

ESI-MS m/ [symbol] : 558.3 [M+H]⁺; >98% ee

¹H NMR (400 MHz, DMSO-d6): δ 9.24 (s, 1H), 8.81 (s, 1H), 8.18 (s, 1H), 8.05-8.02 (m, 1H), 7.85-7.73 (m, 2H), 7.48-7.44 (m, 1H), 7.34 (s, 1H), 7.09 (s, 1H), 4.72 (d, J=10.8 Hz, 1H), 4.08-3.78 (m, 4H), 3.41 (s, 3H), 2.98-2.93 (m, 4H), 2.81-2.71 (m, 1H), 2.66-2.58 (m, 1H), 2.40-2.23 (m, 4H), 1.08 (d, J=6.5 Hz, 3H).

### Example 32

### Synthesis of 2-(1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide

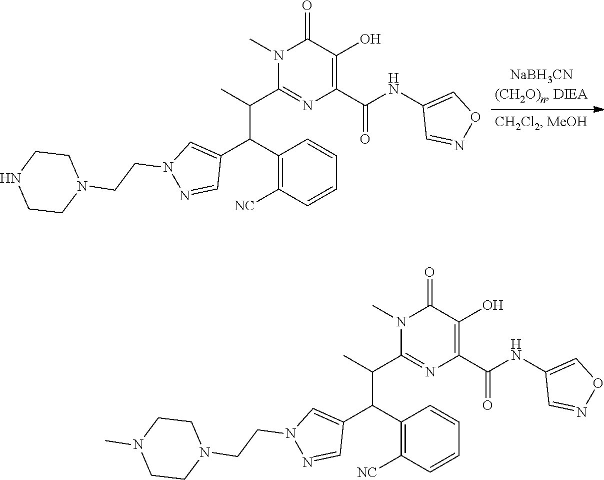 [SMILES: CC(c1nc(C(=O)Nc2cnoc2)c(O)c(=O)n1C)C(c1cnn(CCN2CCNCC2)c1)c1ccccc1C#N ; "NaBH3CN(CH2O)n, DIEA" ; CC(c1nc(C(=O)Nc2cnoc2)c(O)c(=O)n1C)C(c1cnn(CCN2CCN(C)CC2)c1)c1ccccc1C#N]

### Step 1: 2-(1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide

To a 0° C. stirred solution of (2-(1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide) (0.069 mg, 0.1 mmol) in DCM (3 mL) and MeOH (1 mL) was added DIPEA (0.080 mg, 0.6 mmol) and paraformaldehyde (0.112 mg, 1.24 mmol) followed by NaBH₃CN (23.3 mg, 0.4 mmol). The resulting mixture was warmed to rt and stirred for 1 h at which point the crude material was purified by reverse phase chromatography.

Isomer-1_D1E1: Isolated a White solid (0.005 g, 7% yield)

ESI-MS m/ [symbol] : 572.3 [M+H]⁺; >98% ee

¹H NMR (400 MHz, Chloroform-d): δ 11.61 (s, 1H), 9.45 (s, 1H), 9.15 (s, 1H), 8.83 (s, 1H), 7.58-7.46 (m, 5H), 5.07 (d, J=11.2 Hz, 1H), 4.26-4.22 (m, 2H), 3.94-3.87 (m, 1H), 3.73 (s, 3H), 3.13-3.07 (m, 1H), 3.08-2.98 (m, 3H), 2.89 (s, 5H), 2.73 (s, 4H), 1.34 (d, J=6.6 Hz, 3H).

Isomer-2 D1E2: Isolated a White solid (0.010 g, 11% yield)

ESI-MS m/ [symbol] : 572.3 [M+H]⁺; >98% ee

¹H NMR (400 MHz, Chloroform-d): δ 9.45 (s, 1H), 9.15 (s, 1H), 8.82 (s, 1H), 8.38 (s, 1H), 7.58-7.43 (m, 5H), 5.06 (d, J=11.2 Hz, 1H), 4.23-4.20 (t, 2H), 3.85-3.76 (m, 1H), 3.71 (s, 3H), 2.89 (t, 2H), 2.80-2.76 (m, 4H), 2.68-2.61 (m, 5H), 2.51 (s, 3H), 1.35 (d, J=6.6 Hz, 3H).

Isomer-3_D2E1: Isolated a White solid (0.035 g, 31% yield)

ESI-MS m/ [symbol] : 572.3 [M+H]⁺; >98% ee

¹H NMR (400 MHz, Chloroform-d): δ 9.79 (s, 1H), 9.14 (s, 1H), 8.74 (s, 1H), 8.39 (s, 0.65H), 7.76-7.73 (m, 1H), 7.70-7.65 (m, 1H), 7.56-7.52 (m, 1H), 7.47-7.41 (m, 1H), 7.32 (s, 1H), 7.17 (s, 1H), 5.40 (d, J=10.3 Hz, 1H), 4.10 (t, J=6.3 Hz, 2H), 3.63 (s, 4H), 2.85-2.74 (m, 2H), 2.67 (s, 3H), 2.58-2.53 (m, 4H), 2.46 (s, 3H), 1.07 (d, J=6.9 Hz, 3H).

Isomer-4_D2E2: Isolated a White solid (0.027 g, 23% yield)

ESI-MS m/ [symbol] : 572.4 [M+H]⁺; >97% ee

¹H NMR (400 MHz, Chloroform-d): δ 9.79 (s, 1H), 9.14 (s, 1H), 8.74 (s, 1H), 8.39 (s, 0.74H), 7.74 (d, 1H), 7.71-7.65 (m, 1H), 7.54 (d, J=7.9 Hz, 1H), 7.47-7.41 (m, 1H), 7.33 (s, 1H), 7.17 (s, 1H), 5.39 (d, J=10.3 Hz, 1H), 4.10 (t, J=6.3 Hz, 2H), 3.63 (s, 4H), 2.86-2.75 (m, 2H), 2.70 (s, 3H), 2.59-2.57 (m, 4H), 2.49 (s, 3H), 1.07 (d, J=6.8 Hz, 3H).

### Example 33

### Synthesis of 2-(1-(2-cyanophenyl)-1-(1-(2-(dimethylamino)ethyl)-1H-pyrazol-4-yl)propan-2-yl)-5-hydroxy-N-(isoxazol-4-yl)-1-methyl-6-oxo-1,6-dihydropyrimidine-4-carboxamide

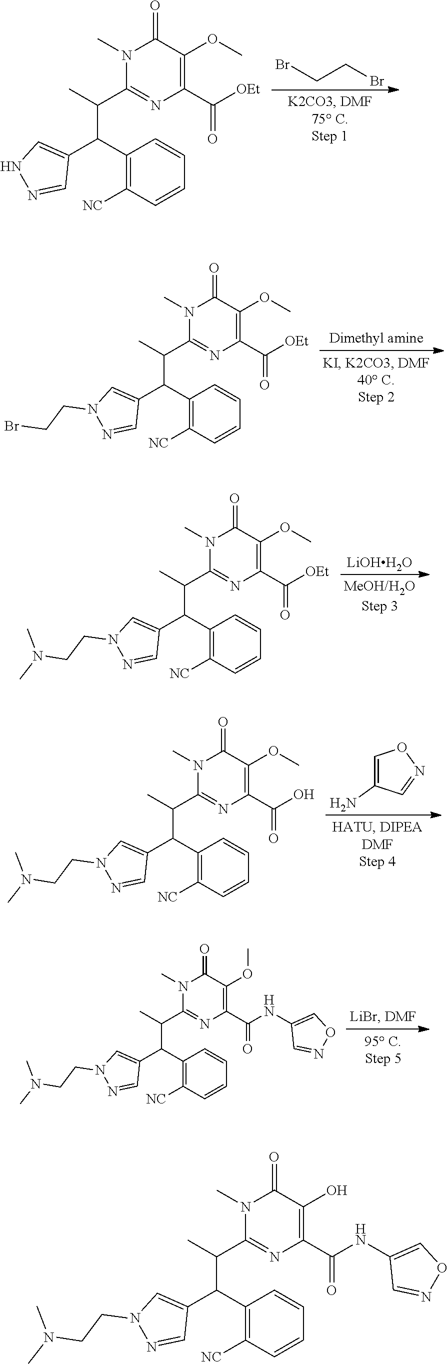

### Step 1: Ethyl 2-[1-[1-(2-bromoethyl)pyrazol-4-yl]-1-(2-cyanophenyl)propan-2-yl]-5-methoxy-1-methyl-6-oxopyrimidine-4-carboxylate

To a stirred solution of ethyl 2-[1-(2-cyanophenyl)-1-(1H-pyrazol-4-yl)propan-2-yl]-5-methoxy-1-methyl-6-oxopyrimidine-4-carboxylate (2.0 g, 4.8 mmol) and K₂CO₃ (2.0 g, 14.3 mmol) in DMF (20 mL) was added dibromoethane (20 mL) at room temperature. The resulting mixture was heated to 75° C. and stirred for 16 h at which point conversion to the desired product was observed by LCMS. The reaction was then cooled to room temperature and diluted with EtOAc (50 mL) and water (100 mL). This solution was then extracted with additional EtOAc (3×50 mL) and the combined organic layers were washed with water (3×50 mL), dried over Na₂SO₄, and concentrated in vacuo. The resulting crude material was purified by silica gel column chromatography (80% EtOAc/petroleum ether) fractions containing product were combined and concentrated to afford the product as a yellow solid (1.25 g, 50% yield)

ESI-MS m/ [symbol] : 528.2/530.3 [M+H]⁺

### Step 2: Ethyl 2-[1-(2-cyanophenyl)-1-[1-[2-(dimethylamino)ethyl]pyrazol-4-yl]propan-2-yl]-5-methoxy-1-methyl-6-oxopyrimidine-4-carboxylate

To a stirred solution of ethyl 2-[1-[1-(2-bromoethyl)pyrazol-4-yl]-1-(2-cyanophenyl)propan-2-yl]-5-methoxy-1-methyl-6-oxopyrimidine-4-carboxylate (500.0 mg, 0.9 mmol), KI (157.1 mg, 0.9 mmol), K₂CO₃ (392.3 mg, 2.8 mmol) in DMF (8 mL) was added dimethylamine (14.2 mL, 14.2 mmol) dropwise at 0° C. The resulting mixture was then heated to 40° C. and stirred overnight at which point conversion to the desired product was observed by LCMS. The mixture was then cooled to 0° C. and water was added. The product was extracted with EtOAc (3×50 mL) and the combined organic layers were washed with water (3×20 mL), dried over Na₂SO₄ then concentrated in vacuo. The resulting crude material was purified by silica gel column chromatography (25% EtOAc/petroleum ether) fractions containing product were combined and concentrated to afford the product as a yellow solid (0.410 g, 88% yield)

ESI-MS m/ [symbol] : 493.2 [M+H]⁺

### Step 3: lithio 2-[1-(2-cyanophenyl)-1-[1-[2-(dimethylamino)ethyl]pyrazol-4-yl]propan-2-yl]-5-methoxy-1-methyl-6-oxopyrimidine-4-carboxylate

To a solution of ethyl 2-[1-(2-cyanophenyl)-1-[1-[2-(dimethylamino)ethyl]pyrazol-4-yl]propan-2-yl]-5-methoxy-1-methyl-6-oxopyrimidine-4-carboxylate (0.520 g, 1.1 mmol) dissolved in MeOH (8 mL) and water (1 mL) was added LiOH·H₂O (0.089 g, 2.1 mmol) portion wise. The resulting mixture was stirred at rt for 2 h at which point it was concentrated in vacuo giving the desired product as a yellow solid (0.550 g) which was used in subsequent steps with no further purification.

ESI-MS m/ [symbol] : 465.2 [M+H]⁺

### Step 4: 2-[1-(2-cyanophenyl)-1-[1-[2-(dimethylamino)ethyl]pyrazol-4-yl]propan-2-yl]-5-methoxy-1-methyl-N-(1,2-oxazol-4-yl)-6-oxopyrimidine-4-carboxamide

To a stirred solution of lithio 2-[1-(2-cyanophenyl)-1-[1-[2-(dimethylamino)ethyl]pyrazol-4-yl]propan-2-yl]-5-methoxy-1-methyl-6-oxopyrimidine-4-carboxylate (0.550 g, 1.2 mmol) and 1,2-oxazol-4-amine hydrochloride (0.282 g, 2.3 mmol in DMF (5 mL) was added HATU (0.889 g, 2.3 mmol) followed by DIPEA (0.302 g, 2.2 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄ and concentrated in vacuo. The resulting crude material was purified by prep-TLC (65% EtOAc/petroleum ether), the product was isolated as a yellow solid (0.500 g, 81% yield).

ESI-MS m/ [symbol] : 531.2 [M+H]⁺

Separation of diastereomers was done at this step using reverse phase C18 chromatography: Column Ultimate XB-C18 Column, 16 um, 50*250 mm; 15% to 60% acetonitrile/water (0.1% FA) in 30 min; Flow rate: 65 mL/min.

Peak 1_D1 contained 175 mg of an off-white solid.

Peak 2_D2 Contained 135 mg of an off-white solid.

Enantiomers of this material were separated by Prep-chiral-HPLC:

D1: Column: CHIRAL ART Cellulose-SC, 2*25 cm, Sum; Mobile Phase A: Hex:MTBE=1:1(0.5% 2M NH3-MeOH), Mobile Phase B:EtOH-HPLC; Flow rate: 20 mL/min; Gradient: 50% B to 50% B in 9 min

Peak 1 (Isomer-1_D1E1): R_{T} 5.39 min; afforded an off-white solid (78 mg)

Peak 2 (Isomer-2_D1E2): R_{T} 6.67 min; afforded an off-white solid (70 mg)

D2: Column: CHIRALPAK IA, 2*25 cm, Sum; Hex:MTBE=1:1(0.5% 2M NH3-MeOH), Mobile Phase B:EtOH-HPLC; Flow rate: 20 mL/min; Gradient: 10% B to 10% B in 22 min).

Peak 1 (Isomer-3_D2E1): R_{T} 12.43 min; afforded a white solid (43 mg)

Peak 2 (Isomer-4_D2E2): R_{T} 17.59 min; afforded a white solid (45 mg)

### Step 5: 2-[1-(2-cyanophenyl)-1-[1-[2-(dimethylamino)ethyl]pyrazol-4-yl]propan-2-yl]-5-hydroxy-1-methyl-N-(1,2-oxazol-4-yl)-6-oxopyrimidine-4-carboxamide

To a solution of 2-[-1-(2-cyanophenyl)-1-[1-[2-(dimethylamino)ethyl]pyrazol-4-yl]propan-2-yl]-5-ethoxy-1-methyl-N-(1,2-oxazol-4-yl)-6-oxopyrimidine-4-carboxamide (0.063 g, 0.1 mmol) dissolved in DMF (3 ml) was added LiBr (0.206 g, 2.4 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated an off-white solid (0.018 g, 29% yield)

ESI-MS m/ [symbol] : 517.4 [M+H]⁺; >98% ee

¹H NMR (300 MHz, DMSO-d6): δ 10.25 (br s, 1H), 8.82 (s, 1H), 8.64 (s, 1H), 7.82-7.76 (m, 2H), 7.57-7.51 (m, 3H), 7.15-7.11 (m, 1H), 4.90 (d, J=11.0 Hz, 1H), 4.14 (t, J=6.6 Hz, 2H), 3.95-3.91 (m, 1H), 3.57 (s, 3H), 2.62 (t, J=6.6 Hz, 2H), 2.14 (s, 6H), 1.33-1.11 (m, 3H).

Isomer-2_D1E2: Isolated an off-white solid (0.014 g, 21% yield)

ESI-MS m/ [symbol] : 517.4 [M+H]⁺; >98% ee

¹H NMR (300 MHz, DMSO-d6): δ 10.26 (br s, 1H), 8.82 (s, 1H), 8.64 (s, 1H), 7.84-7.78 (m, 2H), 7.58-7.51 (m, 3H), 7.15-7.11 (m, 1H), 4.89 (d, J=10.8 Hz, 1H), 4.14 (t, J=6.6 Hz, 2H), 3.95-3.91 (m, 1H), 3.57 (s, 3H), 2.62 (t, J=6.6 Hz, 2H), 2.14 (s, 6H), 1.24-1.16 (m, 3H).

Isomer-3_D2E1: Isolated an off-white solid (0.010 g, 24% yield)

ESI-MS m/ [symbol] : 517.4 [M+H]⁺; >98% ee

¹H NMR (300 MHz, DMSO-d6): δ 11.43 (br s, 1H), 9.16 (s, 1H), 8.68 (s, 1H), 7.96 (d, J=8.0 Hz, 1H), 7.85-7.73 (m, 2H), 7.48-7.44 (m, 2H), 7.06 (s, 1H), 4.85 (d, J=10.6 Hz, 1H), 3.96 (t, J=6.4 Hz, 2H), 3.90-3.83 (m, 1H), 3.50 (s, 3H), 2.47-2.42 (m, 2H), 1.97 (s, 6H), 1.01 (d, J=6.3 Hz, 3H).

Isomer-4_D2E2: Isolated an off-white solid (0.012 g, 26% yield)

ESI-MS m/ [symbol] : 517.3 [M+H]⁺; >98% ee

¹H NMR (300 MHz, DMSO-d6): δ 11.42 (br s, 1H), 9.16 (s, 1H), 8.68 (s, 1H), 7.98-7.95 (m, 1H), 7.85-7.73 (m, 2H), 7.48-7.44 (m, 2H), 7.06 (s, 1H), 4.85 (d, J=10.6 Hz, 1H), 3.96 (t, J=6.4 Hz, 2H), 3.90-3.83 (m, 1H), 3.50 (s, 3H), 2.47-2.42 (m, 2H), 1.97 (s, 6H), 1.02 (d, J=6.3 Hz, 3H).

### Example 34

### Synthesis of 2-(1-(2-cyanophenyl)-1-(1-(3-(dimethylamino)propyl)-1H-pyrazol-4-yl)propan-2-yl)-5-hydroxy-N-(isoxazol-4-yl)-1-methyl-6-oxo-1,6-dihydropyrimidine-4-carboxamide

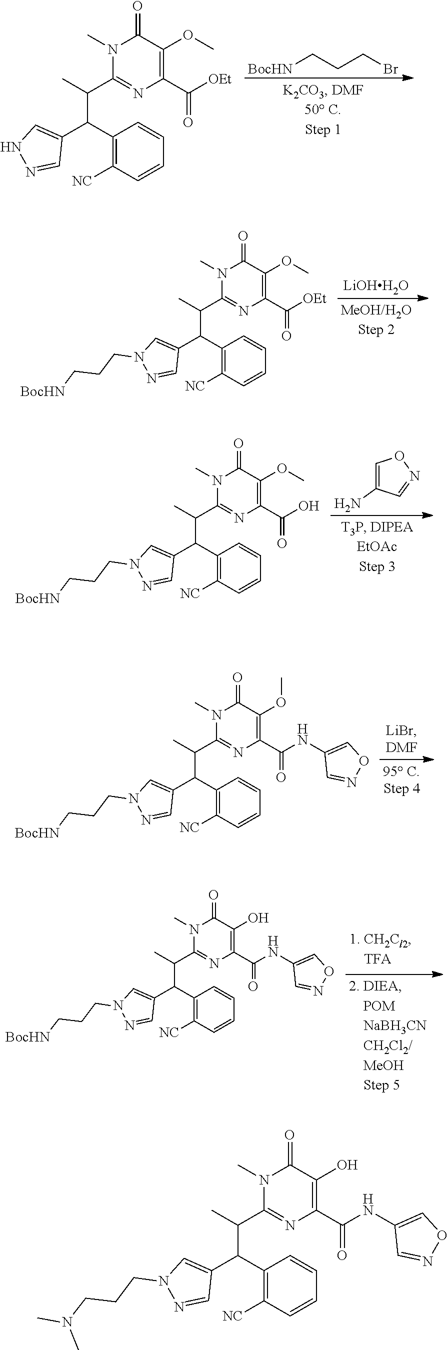

### Step 1: Ethyl 2-[1-(1-{3-[(tert-butoxycarbonyl)amino]propyl}pyrazol-4-yl)-1-(2-cyanophenyl)propan-2-yl]-5-methoxy-1-methyl-6-oxopyrimidine-4-carboxylate

To a stirred solution of ethyl 2-[1-(2-cyanophenyl)-1-(1H-pyrazol-4-yl)propan-2-yl]-5-methoxy-1-methyl-6-oxopyrimidine-4-carboxylate (0.200 g, 0.5 mmol) and K₂CO₃ (0.131 g, 1.0 mmol) in DMF (4 mL) was added tert-butyl N-(3-bromopropyl)carbamate (169.5 mg, 0.7 mmol) at room temperature. The resulting mixture was heated to 50° C. and stirred for 4 h at which point conversion to the desired product was observed by LCMS. The reaction was then cooled to room temperature, diluted with water and the product was extracted with DCM. The organic layer was washed with water, dried over Na₂SO₄ and concentrated in vacuo. The resulting crude material was purified by silica gel column chromatography (EtOAc/petroleum ether). Fractions containing product were combined and concentrated to afford the product as a yellow solid (0.135 g, 49% yield)

ESI-MS m/ [symbol] : 579.3 [M+H]⁺

### Step 2: 2-[1-(1-{3-[(tert-butoxycarbonyl)amino]propyl}pyrazol-4-yl)-1-(2-cyanophenyl)propan-2-yl]-5-methoxy-1-methyl-6-oxopyrimidine-4-carboxylic acid

To a solution of ethyl 2-[1-(1-{3-[(tert-butoxycarbonyl)amino]propyl}pyrazol-4-yl)-1-(2-cyanophenyl)propan-2-yl]-5-methoxy-1-methyl-6-oxopyrimidine-4-carboxylate (1.5 g, 2.6 mmol) dissolved in MeOH (4 mL) and water (20 mL) was added LiOH·H2O (0.218 g, 5.2 mmol) portion wise. The resulting mixture was stirred at rt for 3 h at which point the mixture was brought to pH 4 with HCl (aq). Product was extracted with DCM and the resulting organic layer was collected dried over Na₂SO₄ then concentrated in vacuo giving the desired product as a yellow solid (1.3 g) which was used in subsequent steps with no further purification.

ESI-MS m/ [symbol] : 551.3 [M+H]⁺

### Step 3: tert-butyl N-(3-{4-[1-(2-cyanophenyl)-2-{5-methoxy-1-methyl-4-[(1,2-oxazol-4-yl)carbamoyl]-6-oxopyrimidin-2-yl}propyl]pyrazol-1-yl}propyl)carbamate

To a stirred solution of 2-[1-(1-{3-[(tert-butoxycarbonyl)amino]propyl}pyrazol-4-yl)-1-(2-cyanophenyl)propan-2-yl]-5-methoxy-1-methyl-6-oxopyrimidine-4-carboxylic acid (1.1 g, 2.0 mmol), DIPEA (1.03 g, 8.0 mmol) and 1,2-oxazol-4-amine (0.20 g, 2.4 mmol) in EtOAc (15 ml) was added T₃P (1.27 g, 4.00 mmol) dropwise. The resulting mixture was stirred at rt for 1 h at which point it was diluted with water and product was extracted with EtOAc. The organic layer was then dried over Na₂SO₄ and concentrated in vacuo. Product was isolated by reverse phase chromatography (10% to 80% acetonitrile/water (0.1% FA) fractions containing product were combined and concentrated to afford the product as a light-yellow solid (1.0 g, 81% yield)

ESI-MS m/ [symbol] : 617.3 [M+H]⁺

Separation of diastereomers was done at this step using reverse phase C18 chromatography: Column Sunfire Prep C18 OBD Column, 19*100 mm, 5 m 10 nm; 53% to 85% MeOH/water (0.05% FA) in 30 min; Flow rate: 25 mL/min.

Peak 1_D1 contained 330 mg of an off-white solid.

Peak 2_D2 Contained 415 mg of an off-white solid.

Enantiomers of this material were separated by Prep-chiral-HPLC:

D1: Column: CHIRAL ART Cellulose-SC, 2*25 cm, Sum; Mobile Phase A: Hex:MTBE=1:1 (0.5% 2M NH3-MeOH), Mobile Phase B:EtOH-HPLC; Flow rate: 20 mL/min; Gradient: 10% B to 10% B in 44 min

Peak 1 (Isomer-1_D1E1): R_{T} 29.55 min; afforded a white solid (125 mg)

Peak 2 (Isomer-2_D1E2): R_{T} 37.67 min; afforded a white solid (120 mg)

D2: Column: CHIRALPAK IF, 2*25 cm, 5 um; Hex:MTBE=1:1(10 mM NH3-MeOH), Mobile Phase B:EtOH-HPLC; Flow rate: 20 mL/min; Gradient: 30% B to 30% B in 12.5 min).

Peak 1 (Isomer-3_D2E1): R_{T} 4.87 min; afforded a white solid (145 mg)

Peak 2 (Isomer-4_D2E2): R_{T} 7.78 min; afforded a white solid (148 mg)

### Step 4: tert-butyl N-(3-{4-[(1R,2R)-1-(2-cyanophenyl)-2-{5-methoxy-1-methyl-4-[(1,2-oxazol-4-yl)carbamoyl]-6-oxopyrimidin-2-yl}propyl]pyrazol-1-yl}propyl)carbamate

To a solution of tert-butyl N-(3-{4-[(1R,2R)-1-(2-cyanophenyl)-2-{5-methoxy-1-methyl-4-[(1,2-oxazol-4-yl)carbamoyl]-6-oxopyrimidin-2-yl}propyl]pyrazol-1-yl}propyl)carbamate (0.125 g, 0.2 mmol) dissolved in DMF (5 ml) was added LiBr (0.264 g, 3.0 mmol). This resulting mixture was then heated to 95° C. and stirred for 3 h at which point complete conversion to the product was observed by LCMS. The reaction was then cooled to rt and product was isolated by reverse phase chromatography (10% to 80% acetonitrile/water (0.1% FA). Fractions containing product were combined and concentrated in vacuo to afford the product as a light-yellow solid (0.100 g, 80% yield).

ESI-MS m/ [symbol] : 603.3 [M+H]⁺

### Step 5: 2-[1-(2-cyanophenyl)-1-{1-[3-(dimethylamino)propyl]pyrazol-4-yl}propan-2-yl]-5-hydroxy-1-methyl-N-(1,2-oxazol-4-yl)-6-oxopyrimidine-4-carboxamide

To a 0° C. stirred solution of tert-butyl N-(3-{4-[1-(2-cyanophenyl)-2-{5-hydroxy-1-methyl-4-[(1,2-oxazol-4-yl)carbamoyl]-6-oxopyrimidin-2-yl}propyl]pyrazol-1-yl}propyl)carbamate (0.100 g, 0.2 mmol) in DCM (2 mL) was added TFA (0.5 mL) dropwise. The resulting mixture was warmed to rt and stirred for 1 h at which point Boc deprotection was complete, the reaction was then concentrated in vacuo. The resulting crude material was then dissolved in DCM (2 mL) and MeOH (1 mL) and to this was added N,N-Diisopropylethylamine (0.064 g, 0.5 mmol) and paraformaldehyde (0.120 mg, 1.3 mmol) followed by NaBH₃CN (0.021 g, 0.33 mmol) portion wise. The resulting mixture was stirred at rt for 1 h at which point 0.2 mL of water was added. The reaction mixture was concentrated in vacuo and the crude product was purified by reverse phase chromatography.

Isomer-1_D1E1: Isolated a light-yellow solid (0.012 g, 12% yield).

ESI-MS m/ [symbol] : 531.2 [M+H]⁺; >98% ee

¹H NMR (300 MHz, DMSO-d6): δ 10.95 (s, 1H), 9.27 (s, 1H), 8.85 (s, 1H), 7.78-7.80 (m, 2H), 7.63-7.49 (m, 3H), 7.29-7.13 (m, 1H), 4.97 (d, 1H), 4.17-3.96 (m, 3H), 3.59 (s, 3H), 2.29-2.27 (m, 3H), 2.24-2.20 (m, 5H), 1.92-1.90 (m, 2H), 1.31 (d, 3H).

Isomer-2_D1E2: Isolated an off-white solid (0.009 g, 10% yield)

ESI-MS m/ [symbol] : 531.1 [M+H]⁺; >97% ee

¹H NMR (300 MHz, DMSO-d6): δ 10.96 (s, 1H), 9.27 (s, 1H), 8.85 (s, 1H), 7.78-7.80 (m, 2H), 7.63-7.49 (m, 3H), 7.29-7.13 (m, 1H), 4.97 (d, 1H), 4.17-3.96 (m, 3H), 3.59 (s, 3H), 2.29-2.27 (m, 3H), 2.24-2.20 (m, 5H), 1.92-1.90 (m, 2H), 1.31 (d, 3H).

Isomer-3_D2E1: Isolated an off-white solid (0.012 g, 12% yield)

ESI-MS m/ [symbol] : 531.0 [M+H]⁺; >98% ee

¹H NMR (300 MHz, DMSO-d6): δ 11.33 (s, 1H), 10.51 (s, 1H), 9.35 (s, 1H), 8.92 (s, 1H), 8.03-8.01 (m, 1H), 7.93-7.76 (m, 2H), 7.53-7.50 (m, 1H), 7.41 (s, 1H), 7.16 (s, 1H), 4.79 (d, 1H), 4.15-3.93 (m, 3H), 2.83-2.82 (s, 2H), 2.73-2.72 (m, 6H), 1.98-1.96 (m, 2H), 1.24 (d, 3H).

Isomer-4_D2E2: Isolated an off-white solid (0.014 g, 10% yield)

ESI-MS m/ [symbol] : 531.1 [M+H]⁺; >98% ee

¹H NMR (300 MHz, DMSO-d6): δ 11.33 (s, 1H), 10.51 (s, 1H), 9.35 (s, 1H), 8.92 (s, 1H), 8.03-8.01 (m, 1H), 7.93-7.76 (m, 2H), 7.53-7.50 (m, 1H), 7.41 (s, 1H), 7.16 (s, 1H), 4.79 (d, 1H), 4.15-3.93 (m, 3H), 2.83-2.82 (s, 2H), 2.73-2.72 (m, 6H), 1.98-1.96 (m, 2H), 1.24 (d, 3H).

### Example 35

### Synthesis of 2-(1-(2-cyanophenyl)-1-(1-(2-hydroxyethyl)-1H-pyrazol-4-yl)propan-2-yl)-5-hydroxy-N-(isoxazol-4-yl)-1-methyl-6-oxo-1,6-dihydropyrimidine-4-carboxamide

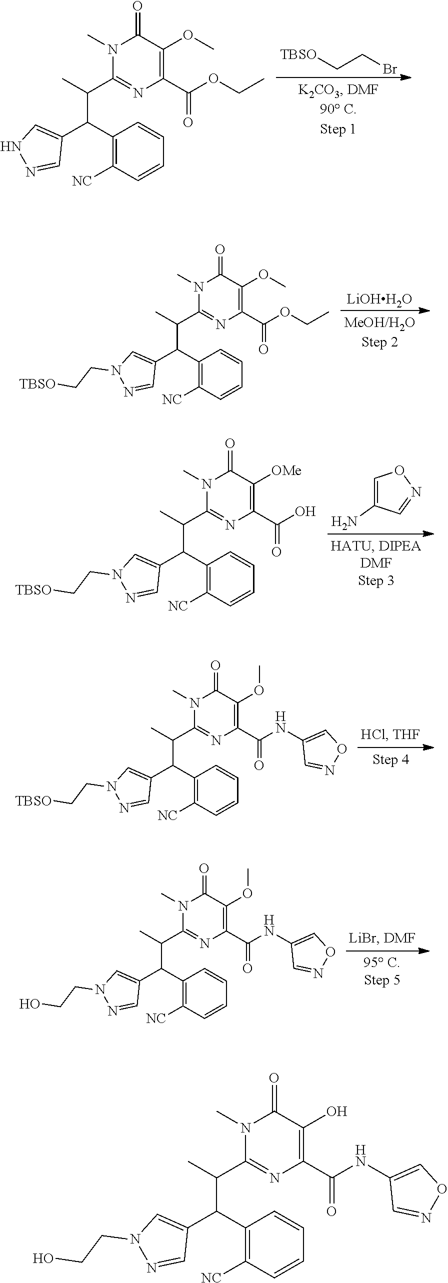

### Step 1: ethyl 2-[1-(1-{2-[(tert-butyldimethylsilyl)oxy]ethyl}pyrazol-4-yl)-1-(2-cyanophenyl)propan-2-yl]-5-methoxy-1-methyl-6-oxopyrimidine-4-carboxylate

To a stirred solution of ethyl 2-[1-(2-cyanophenyl)-1-(1H-pyrazol-4-yl)propan-2-yl]-5-methoxy-1-methyl-6-oxopyrimidine-4-carboxylate (0.100 g, 0.2 mmol) and (2-bromoethoxy)(tert-butyl)dimethylsilane (0.170 mg, 0.7 mmol) in DMF (2 ml) was added K₂CO₃ (0.098 mg, 0.7 mmol). The resulting mixture was heated to 90° C. and stirred overnight at which point the reaction was cooled to room temperature and diluted with water. Product was extracted with DCM and the combined organic layer was washed with brine then dried over Na₂SO₄ and concentrated in vacuo. The resulting crude material was purified by prep-TLC, the product was isolated as a yellow solid (0.090 g, 65% yield).

ESI-MS m/ [symbol] : 580.3 [M+H]⁺.

### Step 2: lithio 2-[1-(1-{2-[(tert-butyldimethylsilyl)oxy]ethyl}pyrazol-4-yl)-1-(2-cyanophenyl)propan-2-yl]-5-methoxy-1-methyl-6-oxopyrimidine-4-carboxylate

To a solution of ethyl 2-[1-(1-{2-[(tert-butyldimethylsilyl)oxy]ethyl}pyrazol-4-yl)-1-(2-cyanophenyl)propan-2-yl]-5-methoxy-1-methyl-6-oxopyrimidine-4-carboxylate (0.300 g, 0.5 mmol) dissolved in MeOH (5 mL) and water (1 mL) was added LiOH·H₂O (0.033 g, 0.8 mmol) portion wise. The resulting mixture was stirred at rt for 3 h at which point it was concentrated in vacuo giving the desired product as a yellow solid (0.280 g) which was used in subsequent steps with no further purification.

ESI-MS m/ [symbol] : 552.4 [M-Li+H]+

### Step 3: 2-[1-(1-{2-[(tert-butyldimethylsilyl)oxy]ethyl}pyrazol-4-yl)-1-(2-cyanophenyl)propan-2-yl]-5-methoxy-1-methyl-N-(1,2-oxazol-4-yl)-6-oxopyrimidine-4-carboxamide

To a stirred solution of lithio 2-[1-(1-{2-[(tert-butyldimethylsilyl)oxy]ethyl}pyrazol-4-yl)-1-(2-cyanophenyl)propan-2-yl]-5-methoxy-1-methyl-6-oxopyrimidine-4-carboxylate (0.280 g, 0.5 mmol) and 1,2-oxazol-4-amine hydrochloride (0.090 g, 0.75 mmol in DMF (5 mL) was added HATU (0.477 g, 1.3 mmol) followed by DIPEA (0.260 g, 2.01 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄, and concentrated in vacuo. The resulting crude material was purified by reverse phase chromatography (10% to 80% acetonitrile/water (0.1% FA) fractions containing product were combined and concentrated to afford the product as a white solid (0.200 g, 64% yield)

[symbol]

ESI-MS m/ [symbol] : 618.3 [M+H]⁺

### Step 4: 2-[1-(2-cyanophenyl)-1-[1-(2-hydroxyethyl)pyrazol-4-yl]propan-2-yl]-5-methoxy-1-methyl-N-(1,2-oxazol-4-yl)-6-oxopyrimidine-4-carboxamide

To a stirred solution of 2-[1-(1-{2-[(tert-butyldimethylsilyl)oxy]ethyl}pyrazol-4-yl)-1-(2-cyanophenyl)propan-2-yl]-5-methoxy-1-methyl-N-(1,2-oxazol-4-yl)-6-oxopyrimidine-4-carboxamide (0.700 mg, 1.1 mmol) in THF (10 mL) was added aq. HCl (2 mL) dropwise. The resulting mixture was stirred for 30 min at room temperature at which point the product was extracted with DCM. The combined organic layers were dried over Na₂SO₄ then concentrated in vacuo.

ESI-MS m/ [symbol] : 504.2 [M+H]⁺

Separation of diastereomers was done at this step using reverse phase chromatography: Column Xselect CSH F-Phenyl OBD column, 19*250, Sum; 61% to 65% MeOH/water (0.1% FA) in 10 min; Flow rate: 25 mL/min.

Peak 1_D1 contained 210 mg of an off-white solid.

Peak 2_D2 Contained 160 mg of an off-white solid.

Enantiomers of this material were separated by Prep-chiral-HPLC:

D1: Column: NB_Lux 5 um i-Cellulose-5, 2.12*25 cm, 5 m; Mobile Phase A: Hex:MTBE=1:1(0.5% 2M NH3-MeOH), Mobile Phase B:EtOH-HPLC; Flow rate: 20 mL/min; Gradient: 20% B to 20% B in 24 min

Peak 1 (Isomer-1_D1E1): R_{T} 16.75 min; afforded a white solid (85 mg)

Peak 2 (Isomer-2_D1E2): R_{T} 20.2 min; afforded a white solid (86 mg)

D2: Column: CHIRAL ART Amylose-SA, 2*25 cm, 5 m; Hex:MTBE=1:1(0.5% 2M NH3-MeOH), Mobile Phase B:EtOH-HPLC; Flow rate: 20 mL/min; Gradient: 20% B to 20% B in 11 min).

Peak 1 (Isomer-3_D2E1): R_{T} 2.00 min; afforded a white solid (58 mg)

Peak 2 (Isomer-4_D2E2): R_{T} 6.00 min; afforded a white solid (25 mg)

### Step 5: 2-[1-(2-cyanophenyl)-1-[1-(2-hydroxyethyl)pyrazol-4-yl]propan-2-yl]-5-hydroxy-1-methyl-N-(1,2-oxazol-4-yl)-6-oxopyrimidine-4-carboxamide

To a solution of 2-[1-(2-cyanophenyl)-1-[1-(2-hydroxyethyl)pyrazol-4-yl]propan-2-yl]-5-methoxy-1-methyl-N-(1,2-oxazol-4-yl)-6-oxopyrimidine-4-carboxamide (0.085 mg, 0.2 mmol) dissolved in DMF (3 ml) was added LiBr (0.220 mg, 2.5 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated a white solid (0.025 g, 30% yield).

ESI-MS m/ [symbol] : 490.2 [M+H]⁺; >98% ee

¹H NMR (300 MHz, DMSO-d6): δ 11.18 (s, 1H), 10.45 (s, 1H), 9.30 (s, 1H), 8.88 (s, 1H), 7.84-7.81 (m, 2H), 7.65-7.50 (m, 3H), 7.21 (t, 1H), 5.00 (d, 1H), 4.87 (t, 1H), 4.10 (t, 2H), 3.71 (q, 2H), 3.60 (s, 3H), 1.33 (d, 3H).

Isomer-2_D1E2: Isolated a white solid (0.026 g, 30% yield)

ESI-MS m/ [symbol] : 490.2 [M+H]⁺; >98% ee

¹H NMR (300 MHz, DMSO-d6): δ 11.18 (s, 1H), 10.46 (s, 1H), 9.30 (s, 1H), 8.88 (s, 1H), 7.84-7.81 (m, 2H), 7.65-7.51 (m, 3H), 7.26-7.15 (m, 1H), 5.00 (d, 1H), 4.87 (t, 1H), 4.11 (t, 3H), 3.71 (q, 2H), 3.60 (s, 3H), 1.33 (d, 3H).

Isomer-3_D2E1: Isolated a white solid (0.020 g, 35% yield)

ESI-MS m/ [symbol] : 490.2 [M+H]⁺; >98% ee

¹H NMR (300 MHz, DMSO-d6): δ 11.23 (s, 1H), 10.40 (s, 1H), 9.33 (s, 1H), 8.88 (s, 1H), 7.96 (d, 1H), 7.85-7.77 (m, 2H), 7.54-7.43 (m, 1H), 7.40 (s, 1H), 7.11 (s, 1H), 4.84 (d, 1H), 4.73 (t, 1H), 4.10-3.91 (m, 3H), 3.54 (q, 2H), 3.46 (s, 3H), 1.16 (d, 3H).

Isomer-4_D2E2: Isolated a white solid (0.021 g, 34% yield)

ESI-MS m/ [symbol] : 490.2 [M+H]⁺; >98% ee

¹H NMR (300 MHz, DMSO-d6): δ 11.23 (s, 1H), 10.40 (s, 1H), 9.33 (s, 1H), 8.88 (s, 1H), 7.96 (d, 1H), 7.88-7.74 (m, 2H), 7.54-7.43 (m, 1H), 7.40 (s, 1H), 7.11 (s, 1H), 4.84 (d, 1H), 4.73 (t, 1H), 4.10-3.91 (m, 3H), 3.54 (q, 2H), 3.46 (s, 3H), 1.16 (d, 3H).

### Example 36

### Synthesis of 2-(1-(2-cyanophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide

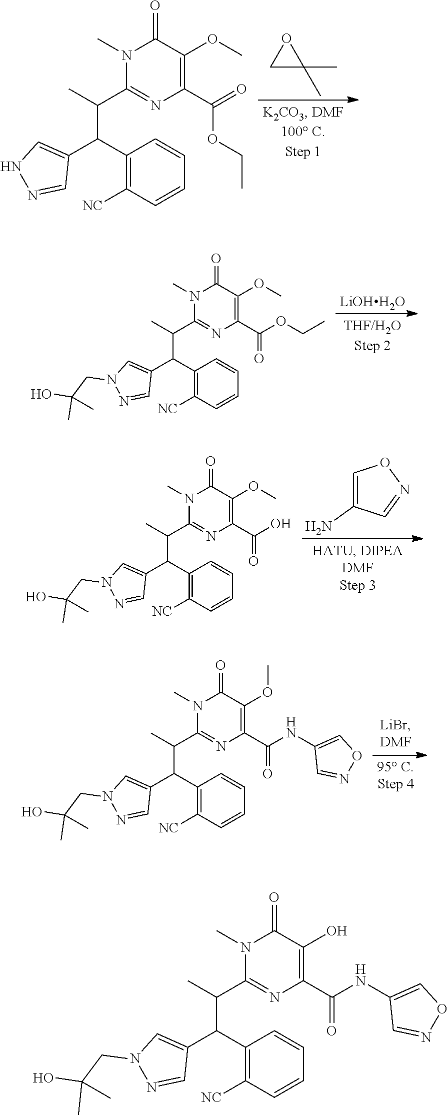

### Step 1: ethyl 2-[1-(2-cyanophenyl)-1-[1-(2-hydroxy-2-methylpropyl)pyrazol-4-yl]propan-2-yl]-5-methoxy-1-methyl-6-oxopyrimidine-4-carboxylate

To a solution of ethyl 2-[1-(2-cyanophenyl)-1-(1H-pyrazol-4-yl)propan-2-yl]-5-methoxy-1-methyl-6-oxopyrimidine-4-carboxylate) (2.0 g, 4.8 mmol) in DMF (20 mL) was added 2,2-dimethyloxirane (0.7 g, 9.5 mmol) and K₂CO₃ (2.0 g, 14.2 mmol). The resulting mixture was heated to 100° C. and stirred overnight. The solution was then cooled to rt, filtered, and purified by reverse phase chromatography (15% to 60% acetonitrile/water (0.1% FA) fractions containing product were combined and concentrated to afford the product as a white solid (1.4 g, 60% yield)

ESI-MS m/ [symbol] : 494.3 [M+H]⁺

### Step 2: 2-(1-(2-cyanophenyl)-1-(1-(2-hydroxy-2-methylpropyl)-1H-pyrazol-4-yl)propan-2-yl)-5-methoxy-1-methyl-6-oxo-1,6-dihydropyrimidine-4-carboxylic acid

To a stirred solution of ethyl 2-[1-(2-cyanophenyl)-1-[1-(2-hydroxy-2-methylpropyl)pyrazol-4-yl]propan-2-yl]-5-methoxy-1-methyl-6-oxopyrimidine-4-carboxylate (1.4 g, 2.8 mmol) in THF (7 mL) was added LiOH·H₂O (0.2 g, 5.7 mmol) in H₂O (7 mL). The resulting mixture was stirred at rt for 2 h at which point it was concentrated in vacuo giving the desired product as a light-yellow solid (0.900 g) which was used in subsequent steps with no further purification

ESI-MS m/ [symbol] : 466.1 [M+H]⁺

### Step 3: 2-[1-(2-cyanophenyl)-1-[1-(2-hydroxy-2-methylpropyl)pyrazol-4-yl]propan-2-yl]-5-methoxy-1-methyl-N-(1,2-oxazol-4-yl)-6-oxopyrimidine-4-carboxamide

To a stirred solution of 2-(1-(2-cyanophenyl)-1-(1-(2-hydroxy-2-methylpropyl)-1H-pyrazol-4-yl)propan-2-yl)-5-methoxy-1-methyl-6-oxo-1,6-dihydropyrimidine-4-carboxylic acid (0.900 g, 1.9 mmol) and 1,2-oxazol-4-amine hydrochloride (0.276 g, 2.3 mmol in DMF (15 mL) was added HATU (1.5 g, 3.8 mmol) followed by DIPEA (0.987 g, 7.6 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄, and concentrated in vacuo. The resulting crude material was purified by reverse phase chromatography (15% to 65% acetonitrile/water (0.1% FA) fractions containing product were combined and concentrated to afford the product as a light-yellow solid (0.726 g, 72% yield).

ESI-MS m/ [symbol] : 532.1 [M+H]⁺

Separation of diastereomers was done at this step using reverse phase C18 chromatography: Column Welch Ultimate AQ-C18 column, 50*250, Sum; 15% to 65% MeOH/water (0.1% FA) in 30 min; Flow rate: 25 mL/min.

Peak 1_D1 contained 285 mg of an off-white solid.

Peak 2_D2 Contained 171 mg of an off-white solid.

Enantiomers of this material were separated by Prep-chiral-HPLC:

D1: Column: CHIRAL ART Cellulose-SB, 2*25 cm, 5 m; Mobile Phase A: Hex:MTBE=1:1(0.5% 2M NH3-MeOH), Mobile Phase B:EtOH-HPLC; Flow rate: 20 mL/min; Gradient: 10% B to 10% B in 34 min

Peak 1 (Isomer-1_D1E1): R_{T} 20.82 min; afforded a white solid (121 mg)

Peak 2 (Isomer-2_D1E2): R_{T} 27.14 min; afforded a white solid (108 mg)

D2: Column: CHIRALPAK IF, 2*25 cm, 5 m; Hex:MTBE=1:1(0.5% 2M NH3-MeOH), Mobile Phase B:EtOH-HPLC; Flow rate: 20 mL/min; Gradient: 30% B to 30% B in 10.5 min).

Peak 1 (Isomer-3_D2E1): R_{T} 5.43 min; afforded a white solid (80 mg)

Peak 2 (Isomer-4_D2E2): R_{T} 7.78 min; afforded a white solid (81 mg)

### Step 4: 2-[1-(2-cyanophenyl)-1-[1-(2-hydroxy-2-methylpropyl)pyrazol-4-yl]propan-2-yl]-5-hydroxy-1-methyl-N-(1,2-oxazol-4-yl)-6-oxopyrimidine-4-carboxamide

To a solution of 2-[1-(2-cyanophenyl)-1-[1-(2-hydroxy-2-methylpropyl)pyrazol-4-yl]propan-2-yl]-5-methoxy-1-methyl-N-(1,2-oxazol-4-yl)-6-oxopyrimidine-4-carboxamide (0.121 mg, 0.2 mmol) dissolved in DMF (5 ml) was added LiBr (0.395 mg, 4.6 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated an off-white solid (0.077 g, 65% yield)

ESI-MS m/ [symbol] : 518.3 [M+H]⁺; >98% ee

¹H NMR (400 MHz, DMSO-d6): δ 11.19 (s, 1H), 10.47 (s, 1H), 9.30 (s, 1H), 8.88 (s, 1H), 7.84-7.79 (m, 2H), 7.62-7.54 (m, 3H), 7.23-7.19 (m, 1H), 5.05-5.02 (d, 1H), 4.12-4.07 (m, 1H), 3.98 (s, 2H), 3.60 (s, 3H), 1.34-1.33 (d, 3H), 1.05 (s, 3H), 0.99 (s, 3H).

Isomer-2_D1E2: Isolated an off-white solid (0.077 g, 73% yield)

ESI-MS m/z: 518.3 [M+H]⁺; >98% ee

¹H NMR (400 MHz, DMSO-d6): δ 11.19 (s, 1H), 10.47 (s, 1H), 9.30 (s, 1H), 8.88 (s, 1H), 7.84-7.79 (m, 2H), 7.62-7.54 (m, 3H), 7.23-7.19 (m, 1H), 5.05-5.02 (d, 1H), 4.12-4.07 (m, 1H), 3.98 (s, 2H), 3.60 (s, 3H), 1.34-1.33 (d, 3H), 1.05 (s, 3H), 0.99 (s, 3H).

Isomer-3_D2E1: Isolated a white solid (0.046 g, 59% yield)

ESI-MS m/ [symbol] : 518.3 [M+H]⁺; >98% ee

¹H NMR (400 MHz, DMSO-d6): δ 11.25 (s, 1H), 10.43 (s, 1H), 9.34 (s, 1H), 8.90 (s, 1H), 8.02-8.00 (m, 1H), 7.85-7.79 (m, 2H), 7.50-7.46 (m, 1H), 7.34 (s, 1H), 7.09 (s, 1H), 4.85-4.83 (d, 1H), 4.09-4.05 (m, 1H), 3.85-3.76 (m, 2H), 3.49 (s, 3H), 1.21-1.19 (d, 3H), 0.84-0.82 (d, 6H).

Isomer-4_D2E2: Isolated a white solid (0.044 g, 56% yield)

ESI-MS m/ [symbol] : 518.3 [M+H]⁺; >98% ee

¹H NMR (400 MHz, DMSO-d6): δ 11.25 (s, 1H), 10.43 (s, 1H), 9.34 (s, 1H), 8.90 (s, 1H), 8.02-8.00 (m, 1H), 7.85-7.79 (m, 2H), 7.50-7.46 (m, 1H), 7.34 (s, 1H), 7.09 (s, 1H), 4.85-4.83 (d, 1H), 4.09-4.05 (m, 1H), 3.85-3.76 (m, 2H), 3.49 (s, 3H), 1.21-1.19 (d, 3H), 0.84-0.82 (d, 6H).

### Example 37

### Synthesis of 2-(1-(2-cyanophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide

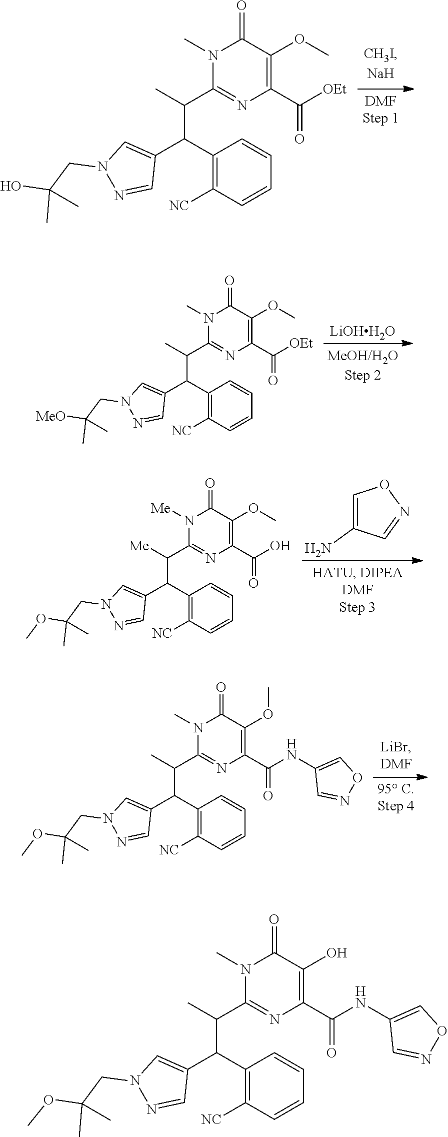

### Step 1: ethyl 2-[1-(2-cyanophenyl)-1-[1-(2-methoxy-2-methylpropyl)pyrazol-4-yl]propan-2-yl]-5-methoxy-1-methyl-6-oxopyrimidine-4-carboxylate

To a 0° C. stirred solution of ethyl 2-[1-(2-cyanophenyl)-1-[1-(2-hydroxy-2-methylpropyl)pyrazol-4-yl]propan-2-yl]-5-methoxy-1-methyl-6-oxopyrimidine-4-carboxylate (2.0 g, 4.1 mmol) in DMF (20 mL) was added sodium hydride (0.2 g, 8.1 mmol) portion wise. After stirring at 0° C. for 30 min iodomethane (0.7 g, 4.9 mmol) was added and the resulting mixture was warmed to rt then stirred for 1 h. The reaction was quenched by the addition of sat. NH₄Cl (aq., 40 mL) and the product was extracted with EtOAc (3×15 mL). The combined organic layers were washed with brine (3×20 mL), dried over Na₂SO₄, and concentrated in vacuo. The crude material was purified by reverse phase chromatography (20% to 60% acetonitrile/water (0.1% FA) fractions containing product were combined and concentrated to afford the product as a light-yellow solid (1.6 g, 78% yield).

ESI-MS m/ [symbol] : 508.3 [M+H]⁺

### Step 2: 2-[1-(2-cyanophenyl)-1-[1-(2-methoxy-2-methylpropyl)pyrazol-4-yl]propan-2-yl]-5-methoxy-1-methyl-6-oxopyrimidine-4-carboxylate

To a stirred solution of ethyl 2-[1-(2-cyanophenyl)-1-[1-(2-methoxy-2-methylpropyl)pyrazol-4-yl]propan-2-yl]-5-methoxy-1-methyl-6-oxopyrimidine-4-carboxylate (1.6 g, 3.15 mmol) in THF (10 mL) was added LiOH·H₂O (0.3 g, 6.3 mmol) in H₂O (10 mL). The resulting mixture was stirred at rt for 3 h at which point the mixture was brought to pH 5 with HCl (aq). Product was extracted with EtOAc and the resulting organic layer was collected dried over Na₂SO₄ then concentrated in vacuo. The crude material was purified by reverse phase chromatography (10% to 50% acetonitrile/water (0.1% FA) fractions containing product were combined and concentrated to afford the product as a light-yellow solid (0.843 g, 55% yield)

ESI-MS m/ [symbol] : 480.3 [M+H]⁺

### Step 3: 2-[1-(2-cyanophenyl)-1-[1-(2-methoxy-2-methylpropyl)pyrazol-4-yl]propan-2-yl]-5-methoxy-1-methyl-N-(1,2-oxazol-4-yl)-6-oxopyrimidine-4-carboxamide

To a stirred solution of 2-[1-(2-cyanophenyl)-1-[1-(2-methoxy-2-methylpropyl)pyrazol-4-yl]propan-2-yl]-5-methoxy-1-methyl-6-oxopyrimidine-4-carboxylate (0.843 g, 1.8 mmol) and 1,2-oxazol-4-amine hydrochloride (0.254 g, 2.1 mmol in DMF (15 mL) was added HATU (1.3 g, 3.5 mmol) followed by DIPEA (0.909 g, 7.0 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄, and concentrated in vacuo. The resulting crude material was purified by reverse phase chromatography (20% to 65% acetonitrile/water (0.1% FA) fractions containing product were combined and concentrated to afford the product as a light-yellow solid (0.722 g, 75% yield).

ESI-MS m/ [symbol] : 546.2 [M+H]⁺

Separation of diastereomers was done at this step using reverse phase C18 chromatography: Column Welch Ultimate AQ-C18 column, 50*250, Sum; 15% to 60% MeOH/water (0.1% FA) in 30 min; Flow rate: 25 mL/min.

Peak 1_D1 contained 324 mg of a light-yellow solid.

Peak 2_D2 Contained 250 mg of a light-yellow solid.

Enantiomers of this material were separated by Prep-chiral-HPLC:

D1: Column: CHIRAL ART Cellulose-SB, 2*25 cm, 5 m; Mobile Phase A: Hex:MTBE=1:1 (10 mM NH3-MeOH), Mobile Phase B:EtOH-HPLC; Flow rate: 20 mL/min; Gradient: 30% B to 30% B in 8 min

Peak 1 (Isomer-1_D1E1): R_{T} 4.63 min; afforded a light-yellow solid (105 mg)

Peak 2 (Isomer-2_D1E2): R_{T} 6.13 min; afforded a light-yellow solid (118 mg)

D2: Column: CHIRALPAK IF, 2*25 cm, 5 m; Hex:MTBE=1:1(0.5% 2M NH₃-MeOH), Mobile Phase B:EtOH-HPLC; Flow rate: 20 mL/min; Gradient: 30% B to 30% B in 13.5 min).

Peak 1 (Isomer-3_D2E1): R_{T} 6.62 min; afforded a light-yellow solid (95 mg).

Peak 2 (Isomer-4_D2E2): R_{T} 10.61 min; afforded a light-yellow solid (100 mg).

### Step 4: 2-[1-(2-cyanophenyl)-1-[1-(2-methoxy-2-methylpropyl)pyrazol-4-yl]propan-2-yl]-5-hydroxy-1-methyl-N-(1,2-oxazol-4-yl)-6-oxopyrimidine-4-carboxamide

To a solution of 2-[1-(2-cyanophenyl)-1-[1-(2-methoxy-2-methylpropyl)pyrazol-4-yl]propan-2-yl]-5-methoxy-1-methyl-N-(1,2-oxazol-4-yl)-6-oxopyrimidine-4-carboxamide (0.105 mg, 0.2 mmol) dissolved in DMF (5 ml) was added LiBr (0.334 g, 3.8 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated an off-white solid (0.055 g, 54% yield).

ESI-MS m/ [symbol] : 532.2 [M+H]⁺; >98% ee

¹H NMR (400 MHz, DMSO-d6): δ 11.19 (s, 1H), 10.48 (s, 1H), 9.30 (s, 1H), 8.88 (s, 1H), 7.83-7.81 (m, 1H), 7.75 (s, 1H), 7.60-7.54 (m, 3H), 7.22-7.19 (m, 1H), 5.03-5.00 (d, 1H), 4.09-4.08 (m, 3H), 3.61 (s, 3H), 3.16 (s, 3H), 1.34-1.32 (d, 3H), 1.05-1.02 (d, 6H).

Isomer-2_D1E2: Isolated an off-white solid (0.064 g, 55% yield)

ESI-MS m/ [symbol] : 532.2 [M+H]⁺; >98% ee

¹H NMR (400 MHz, DMSO-d6): δ 11.19 (s, 1H), 10.48 (s, 1H), 9.30 (s, 1H), 8.88 (s, 1H), 7.83-7.81 (m, 1H), 7.75 (s, 1H), 7.60-7.54 (m, 3H), 7.22-7.19 (m, 1H), 5.03-5.00 (d, 1H), 4.09-4.08 (m, 3H), 3.61 (s, 3H), 3.16 (s, 3H), 1.34-1.32 (d, 3H), 1.05-1.02 (d, 6H).

Isomer-3_D2E1: Isolated a white solid (0.037 g, 39% yield)

ESI-MS m/ [symbol] : 532.2 [M+H]⁺; >98% ee

¹H NMR (400 MHz, DMSO-d6): δ 11.25 (s, 1H), 10.49 (s, 1H), 9.34 (s, 1H), 8.90 (s, 1H), 8.04-8.02 (m, 1H), 7.85-7.79 (m, 2H), 7.50-7.46 (m, 1H), 7.26 (s, 1H), 7.10 (s, 1H), 4.86-4.83 (d, 1H), 4.10-4.06 (m, 1H), 3.94-3.86 (m, 2H), 3.50 (s, 3H), 2.99 (s, 3H), 1.22-1.20 (d, 3H), 0.83-0.81 (d, 6H).

Isomer-4_D2E2: Isolated a white solid (0.045 g, 46% yield)

ESI-MS m/ [symbol] : 532.2 [M+H]⁺; >98% ee

¹H NMR (400 MHz, DMSO-d6): δ 11.25 (s, 1H), 10.49 (s, 1H), 9.34 (s, 1H), 8.90 (s, 1H), 8.04-8.02 (m, 1H), 7.85-7.79 (m, 2H), 7.50-7.46 (m, 1H), 7.26 (s, 1H), 7.10 (s, 1H), 4.86-4.83 (d, 1H), 4.10-4.06 (m, 1H), 3.94-3.86 (m, 2H), 3.50 (s, 3H), 2.99 (s, 3H), 1.22-1.20 (d, 3H), 0.83-0.81 (d, 6H).

### Example 38

### Synthesis of 2-(1-(2-cyanophenyl)-1-(1-(difluoromethyl)-1H-pyrazol-4-yl)propan-2-yl)-5-hydroxy-N-(isoxazol-4-yl)-1-methyl-6-oxo-1,6-dihydropyrimidine-4-carboxamide

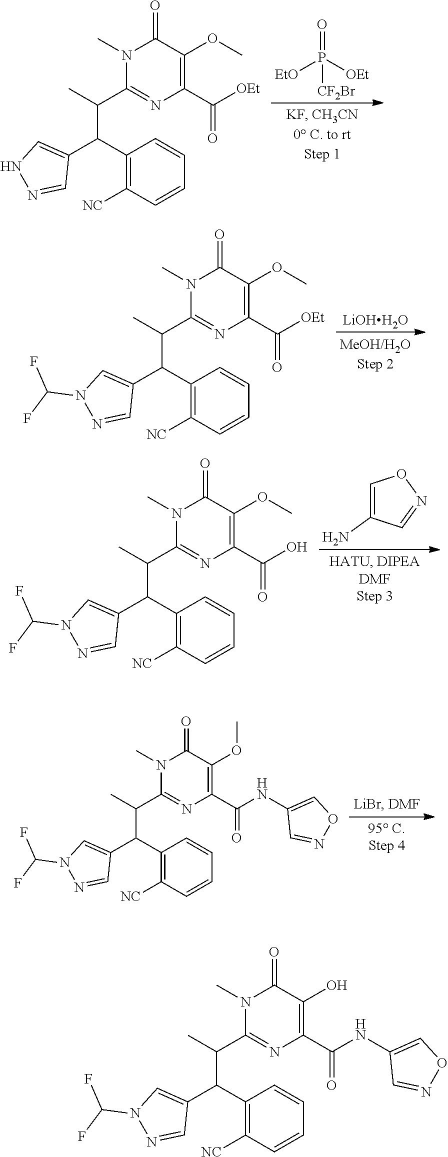

### Step 1: ethyl 2-(1-(2-cyanophenyl)-1-(1-(difluoromethyl)-1H-pyrazol-4-yl)propan-2-yl)-5-methoxy-1-methyl-6-oxo-1,6-dihydropyrimidine-4-carboxylate

To a 0° C. stirred mixture of ethyl 2-[1-(2-cyanophenyl)-1-(1H-pyrazol-4-yl)propan-2-yl]-5-methoxy-1-methyl-6-oxopyrimidine-4-carboxylate (1.0 g, 2.4 mmol) and KF (0.21 g, 3.6 mmol) in acetonitrile (10 mL) was added diethyl (bromodifluoromethyl)phosphonate (0.95 g, 3.6 mmol) dropwise. The resulting mixture was warmed to rt and stirred overnight at which point the reaction was diluted with H₂O and the product was extracted with EtOAc. The organic layers were combined, washed with brine, dried over Na₂SO₄ then concentrated in vacuo giving the desired product (1.20 g) which was used in subsequent steps with no further purification

ESI-MS m/ [symbol] : 572.2 [M+H]⁺

### Step 2: 2-(1-(2-cyanophenyl)-1-(1-(difluoromethyl)-1H-pyrazol-4-yl)propan-2-yl)-5-methoxy-1-methyl-6-oxo-1,6-dihydropyrimidine-4-carboxylic acid

To a 0° C. stirred solution of ethyl 2-[1-(2-cyanophenyl)-1-[1-(difluoromethyl)pyrazol-4-yl]propan-2-yl]-5-methoxy-1-methyl-6-oxopyrimidine-4-carboxylate (1.7 g, 3.6 mmol) in THF (15 mL) was added LiOH·H2O (0.23 g, 5.4 mmol) in H₂O (3 mL). The resulting solution was warmed to rt and stirred for 3 h at which point the mixture was brought to pH 5 with HCl (aq) and the product was extracted with DCM. The resulting organic layer was washed with H2O, dried over Na₂SO₄, and concentrated in vacuo giving the desired product as a light-yellow solid (0.920 g) which was used in subsequent steps with no further purification.

ESI-MS m/ [symbol] : 441.1 [M+H]⁺

### Step 3: 2-[1-(2-cyanophenyl)-1-[1-(difluoromethyl)pyrazol-4-yl]propan-2-yl]-5-methoxy-1-methyl-N-(1,2-oxazol-4-yl)-6-oxopyrimidine-4-carboxamide

To a 0° C. stirred solution of 2-(1-(2-cyanophenyl)-1-(1-(difluoromethyl)-1H-pyrazol-4-yl)propan-2-yl)-5-methoxy-1-methyl-6-oxo-1,6-dihydropyrimidine-4-carboxylic acid (1.23 g, 2.73 mmol) and 1,2-oxazol-4-amine hydrochloride (0.361 g, 3.0 mmol) in DMF (12 mL) was added HATU (1.35 g, 3.6 mmol) followed by DIPEA (1.77 g, 13.7 mmol) dropwise. The resulting mixture was warmed to rt and stirred for 1.5 h at which point it was purified by reverse phase chromatography (0% to 100% acetonitrile/water (0.1% FA)) fractions containing product were combined and concentrated to afford the product as a light-yellow solid (1.20 g, 85% yield)

ESI-MS m/ [symbol] : 510.4 [M+H]⁺

Separation of diastereomers was done at this step using reverse phase C18 chromatography: Column XBridge Shield RP18 OBD Column, 19*150 mm, 5 m; 34% to 37% MeOH/water (10 mM NH4HCO3) in 8 min; Flow rate: 25 mL/min.

Peak 1_D1 contained 700 mg of a light-yellow solid

Peak 2_D2 Contained 300 mg of a light-yellow solid

Enantiomers of this material were separated by Prep-chiral-HPLC:

D1: Column: CHIRAL ART Cellulose-SC, 2*25 cm, 5 m; Mobile Phase A: Hex:MTBE=1:1 (0.5% 2 mM NH3-MeOH), Mobile Phase B:EtOH-HPLC; Flow rate: 20 mL/min; Gradient: 10% B to 10% B in 13 min

Peak 1 (Isomer-1_D1E1): R_{T} 8.37 min; afforded a light-yellow solid (285 mg)

Peak 2 (Isomer-2_D1E2): R_{T} 10.82 min; afforded a light-yellow solid (260 mg)

D2: Column: CHIRALPAK ID, 2*25 cm, 5 m; Hex:MTBE=1:1 (0.1% DEA), Mobile Phase B:EtOH-HPLC; Flow rate: 20 mL/min; Gradient: 30% B to 30% B in 4 min).

Peak 1 (Isomer-3_D2E1): R_{T} 1.45 min; afforded a light-yellow solid (145 mg)

Peak 2 (Isomer-4_D2E2): R_{T} 2.51 min; afforded a light-yellow solid (110 mg)

### Step 4: 2-[(1-(2-cyanophenyl)-1-[1-(difluoromethyl)pyrazol-4-yl]propan-2-yl]-5-hydroxy-1-methyl-N-(1,2-oxazol-4-yl)-6-oxopyrimidine-4-carboxamide

To a solution of 2-[(1-(2-cyanophenyl)-1-[1-(difluoromethyl)pyrazol-4-yl]propan-2-yl]-5-methoxy-1-methyl-N-(1,2-oxazol-4-yl)-6-oxopyrimidine-4-carboxamide (0.145 mg, 0.3 mmol) dissolved in DMF (5 ml) was added LiBr (0.494 g, 5.7 mmol). This resulting mixture was then heated to 95° C. and stirred for 2 h at which point complete conversion to the product was observed by LCMS. The reaction was then cooled to rt and concentrated in vacuo. The resulting crude material was purified by reverse phase chromatography.

Isomer-1_D1E1: Isolated a white solid (0.141 g, 50% yield)

ESI-MS m/ [symbol] : 496.1 [M+H]⁺; >98% ee

¹H NMR (400 MHz, DMSO-d6): δ 11.17 (s, 1H), 10.48 (s, 1H), 9.29 (s, 1H), 8.87 (s, 1H), 8.38 (s, 1H), 7.99-7.80 (m, 3H), 7.65-7.57 (m, 2H), 7.23 (t, 1H), 5.10 (d, 1H), 4.22-4.18 (m, 1H), 3.61 (s, 3H), 1.33 (d, 3H).

Isomer-2_D1E2: Isolated a white solid (0.093 g, 36% yield)

ESI-MS m/ [symbol] : 496.1 [M+H]⁺; >98% ee

¹H NMR (400 MHz, DMSO-d6): δ 11.17 (s, 1H), 10.49 (s, 1H), 9.30 (s, 1H), 8.87 (s, 1H), 8.38 (s, 1H), 7.97 (d, 1H), 7.86-7.81 (m, 2H), 7.66-7.58 (m, 2H), 7.25 (t, 1H), 5.10 (d, 1H), 4.23-4.20 (m, 1H), 3.60 (s, 3H), 1.33 (d, 3H).

Isomer-3_D2E1: Isolated a white solid (0.052 g, 36% yield)

ESI-MS m/ [symbol] : 496.1 [M+H]⁺; >98% ee

¹H NMR (400 MHz, DMSO-d6): δ 11.24 (s, 1H), 10.38 (s, 1H), 9.37 (s, 1H), 8.89 (s, 1H), 8.10-7.78 (m, 4H), 7.63-7.49 (m, 3H), 4.98 (d, 1H), 4.09-4.06 (m, 1H), 3.52 (s, 3H), 1.18-1.16 (m, 3H).

Isomer-4_D2E2: Isolated a white solid (0.047 g, 44% yield)

ESI-MS m/ [symbol] : 496.1 [M+H]⁺; >98% ee

¹H NMR (400 MHz, DMSO-d6): δ 11.23 (s, 1H), 10.32 (s, 1H), 9.33 (s, 1H), 8.86 (s, 1H), 8.00 (d, 2H), 7.88-7.77 (m, 2H), 7.62-7.48 (m, 3H), 5.01 (d, 1H), 4.11-4.06 (m, 1H), 3.53 (s, 3H), 1.17 (d, 3H).

### Example 39

### Synthesis of 2-(1-(2-cyanophenyl)-1-(1-methyl-1H-imidazol-4-yl)propan-2-yl)-5-hydroxy-N-(isoxazol-4-yl)-1-methyl-6-oxo-1,6-dihydropyrimidine-4-carboxamide

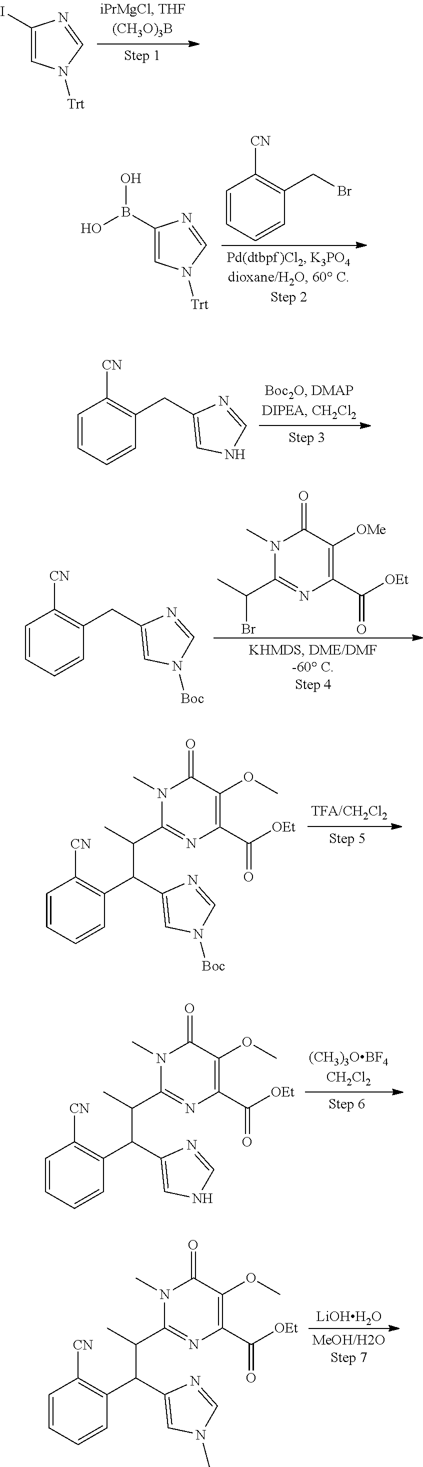

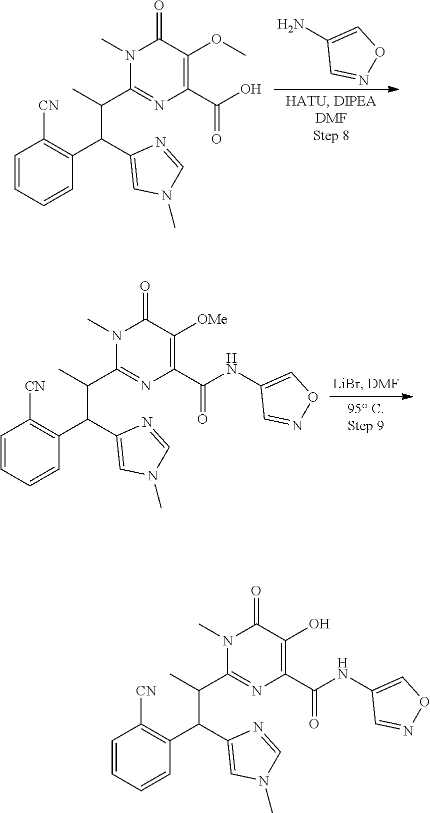 [SMILES: COc1c(C(=O)O)nc(C(C)C(c2cn(C)cn2)c2ccccc2C#N)n(C)c1=O ; Nc1cnoc1 ; COc1c(C(=O)Nc2cnoc2)nc(C(C)C(c2cn(C)cn2)c2ccccc2C#N)n(C)c1=O ; CC(c1nc(C(=O)Nc2cnoc2)c(O)c(=O)n1C)C(c1cn(C)cn1)c1ccccc1C#N]

### Step 1: 1-(triphenylmethyl)imidazol-4-ylboronic acid

To a stirred solution of 4-iodo-1-(triphenylmethyl)imidazole (6.6 g, 15 mmol) in THF (200 mL) was added chloro(isopropyl)magnesium (9 mL, 177 mmol) dropwise at 0° C. This mixture was stirred for 30 min at 0° C. at which point trimethyl borate (2.36 g, 22.7 mmol) was added dropwise over 5 min at 0° C. The resulting mixture was then warmed to rt and stirred for 30 min at which point it was re-cooled to 0° C. and quenched by the addition of sat. ammonium chloride (aq.) (50 mL). The product was extracted with EtOAc (2×100 mL) and the organic layers were combined, washed with brine, dried over Na₂SO₄ then concentrated in vacuo giving the desired product as an off-white solid which was used in subsequent steps with no further purification.

ESI-MS m/ [symbol] : 572.2 [M+H]⁺

### Step 2: 2-(1H-imidazol-4-ylmethyl)benzonitrile

To a stirred mixture of 2-(bromomethyl)benzonitrile (2.0 g, 10 mmol) and (1-(triphenylmethyl)imidazol-4-ylboronic acid) (4.7 g, 13 mmol), in 1,2-Dimethoxyethane (50 mL) and water (10 mL) were added Potassium phosphate tribasic hydrate (4.33 g, 20 mmol) and Dichloro[1,1′-bis(di-t-butylphosphino)ferrocene]palladium(II) (0.532 g, 0.8 mmol). The resulting mixture was heated to 60° C. and stirred for 2 h then allowed to cool down to room temperature and diluted with water. Product was extracted with EtOAc and the organic layers were combined, washed with brine, dried over Na₂SO₄ then concentrated in vacuo. The resulting mixture was purified by HPLC Silica gel column (10% to 100% acetonitrile/water) fractions containing product were combined and concentrated to afford the product as a yellow oil (1.20 g, 64% yield)

ESI-MS m/ [symbol] : 184.2 [M+H]⁺

### Step 3: tert-butyl 4-[(2-cyanophenyl)methyl]imidazole-1-carboxylate

To a 0° C. stirred solution of 2-(1H-imidazol-4-ylmethyl)benzonitrile (1.2 g, 6.55 mmol) in DCM was added di-tert-butyl dicarbonate (2.86 g, 13 mmol) followed by 4-Dimethylaminopyridine (0.08 g, 0.7 mmol) and DIPEA (2.54 g, 19.7 mmol) dropwise. The resulting mixture was then warmed to rt and stirred for 2 h at which point it was cooled to 0° C. and quenched with water. Product was extracted with DCM (3×30 mL) and the combined organic layers were washed with brine, dried over Na₂SO₄ then concentrated in vacuo. The resulting crude reaction material was purified by silica gel column chromatography (0-50% EtOAc/pet. ether) fractions containing product were combined and concentrated to afford the product as a yellow oil (0.801 g, 43% yield)

ESI-MS m/ [symbol] : 284.2 [M+H]⁺

¹H NMR (400 MHz, DMSO-d6): δ 7.91 (d, J=7.7, 1.4 Hz, 1H), 7.66 (t, J=7.7 Hz, 1.4 Hz, 1H), 7.54-7.46 (m, 2H), 7.18 (d, J=1.0 Hz, 1H), 6.68 (d, J=7.9 Hz, 1H), 5.81 (s, 2H), 1.37 (s, 9H).

### Step 4: (ethyl 2-{1-[1-(tert-butoxycarbonyl)imidazol-4-yl]-1-(2-cyanophenyl)propan-2-yl}-5-methoxy-1-methyl-6-oxopyrimidine-4-carboxylate)

To a −65° C. stirred solution of (tert-butyl 4-[(2-cyanophenyl)methyl]imidazole-1-carboxylate) (0.830 g, 2.9 mmol), ethyl 2-(1-bromoethyl)-5-methoxy-1-methyl-6-oxopyrimidine-4-carboxylate (0.935 g, 2.9 mmol) in DMF (3 mL) and 1,2-Dimethoxyethane (6 mL) was added Potassium bis(trimethylsilyl)amide (0.87 mL, 3.8 mmol) dropwise over 5 min. The resulting mixture was stirred for 40 min at −65° C. at which point formation of the desired product was observed by LCMS at which point the reaction was quenched at −65° C. with sat. ammonium chloride (aq.). Product was extracted with EtOAc (3×100 mL) and the combined organic layers were washed with water then brine, dried over Na₂SO₄ and concentrated in vacuo. The resulting mixture was purified by HPLC Silica gel column (10% to 100% acetonitrile/water) fractions containing product were combined and concentrated to afford the product as a yellow solid (0.502 g, 33% yield)

ESI-MS m/ [symbol] : 522.3 [M+H]⁺

### Step 5: ethyl 2-[1-(2-cyanophenyl)-1-(1H-imidazol-4-yl)propan-2-yl]-5-methoxy-1-methyl-6-oxopyrimidine-4-carboxylate

To a stirred solution of ethyl 2-{1-[1-(tert-butoxycarbonyl)imidazol-4-yl]-1-(2-cyanophenyl)propan-2-yl}-5-methoxy-1-methyl-6-oxopyrimidine-4-carboxylate (0.810 g, 1.6 mmol) in DCM (10 mL). This solution was cooled to 0° C. and TFA (3 mL) was added dropwise. The resulting mixture was warmed to rt and stirred for 2 hr at room temperature then concentrated in vacuo. The resulting crude reaction material was purified by silica gel column chromatography (3% MeOH/DCM) fractions containing product were combined and concentrated to afford the product as a yellow solid (0.610 g, 93% yield)

ESI-MS m/ [symbol] : 422.2 [M+H]⁺

### Step 6: ethyl 2-[1-(2-cyanophenyl)-1-(1-methylimidazol-4-yl)propan-2-yl]-5-methoxy-1-methyl-6-oxopyrimidine-4-carboxylate

To a stirred solution of ethyl 2-[1-(2-cyanophenyl)-1-(1H-imidazol-4-yl)propan-2-yl]-5-methoxy-1-methyl-6-oxopyrimidine-4-carboxylate (0.410 g, 1.0 mmol), in DCM (5 mL) was added Trimethyloxonium tetrafluoroborate (0.173 mg, 1.2 mmol). The resulting mixture was stirred for 2 h at room temperature at which point conversion to the desired product was observed by LCMS. The reaction was then cooled to 0° C. and quenched with H₂O. Product was extracted with DCM (3×30 mL) and the combined organic layers were washed with water then brine, dried over Na₂SO₄ and concentrated in vacuo. The resulting mixture was purified by Prep-TLC (50% EtOAc/pet. Ether) to afford the product as a yellow solid (0.310 g, 70% yield)

ESI-MS m/ [symbol] : 436.2 [M+H]⁺

### Step 7: 2-[1-(2-cyanophenyl)-1-(1-methylimidazol-4-yl)propan-2-yl]-5-methoxy-1-methyl-6-oxopyrimidine-4-carboxylate

To a solution of ethyl 2-[1-(2-cyanophenyl)-1-(1-methylimidazol-4-yl)propan-2-yl]-5-methoxy-1-methyl-6-oxopyrimidine-4-carboxylate (0.310 g, 0.7 mmol) dissolved in MeOH (5 mL) and water (1 mL) was added LiOH·H₂O (0.060 g, 1.4 mmol) portion wise. The resulting mixture was stirred at rt for 2 h at which point it was concentrated in vacuo giving the desired product as a yellow solid (0.330 g) which was used in subsequent steps with no further purification.

ESI-MS m/ [symbol] : 408.1 [M+H]⁺

### Step 8: 2-[1-(2-cyanophenyl)-1-(1-methylimidazol-4-yl)propan-2-yl]-5-methoxy-1-methyl-N-(1,2-oxazol-4-yl)-6-oxopyrimidine-4-carboxamide

To a stirred solution of 2-[1-(2-cyanophenyl)-1-(1-methylimidazol-4-yl)propan-2-yl]-5-methoxy-1-methyl-6-oxopyrimidine-4-carboxylic acid (0.270 g, 0.7 mmol) and 1,2-oxazol-4-amine hydrochloride (0.067 g, 0.8 mmol) in DMF (5 mL) was N,N diisopropylethyl amine (0.257 g, 2.0 mmol) dropwise followed by 2,4,6-Tripropyl-2,4,6-trioxo-1,3,5,2,4,6-trioxatriphosphorinane (0.422 mg, 1.3 mmol). The resulting mixture was stirred at rt for 1 h at which point it was diluted with water (20 mL) and the product was extracted with EtOAc (3×50 mL). The organic layers were collected and combined then washed with brine, dried over Na₂SO₄, and concentrated in vacuo. The resulting crude material was purified by prep-TLC (7% MeOH/DCM), the product was isolated as a yellow solid (0.305 g, 97% yield).

ESI-MS m/ [symbol] : 474.2 [M+H]⁺

Separation of diastereomers was done at this step using reverse phase C18 chromatography: Column Xselect CSH F-Phenyl OBD, 5 um, 19*250 mm; 17% to 24% acetonitrile/water (0.1% FA) in 10 min; Flow rate: 25 mL/min.

Peak 1_D1 contained 95 mg of an off-white solid

Peak 2_D2 Contained 85 mg of an off-white solid

Enantiomers of this material were separated by Prep-chiral-HPLC:

D1: Column: Chiralpak IE, 2*25 cm, Sum; Mobile Phase A: Hex:MTBE=1:1(0.1% TFA), Mobile Phase B:EtOH-HPLC; Flow rate: 20 mL/min; Gradient: 40% B to 40% B in 10.5 min

Peak 1 (Isomer-1_D1E1): R_{T} 5.27 min; afforded an off-white solid (40 mg)

Peak 2 (Isomer-2_D1E2): R_{T} 7.11 min; afforded an off-white solid (42 mg)

D2: Column: CHIRALPAK IE, 2*25 cm, Sum; Hex:MTBE=1:1(0.5% 2M NH₃-MeOH), Mobile Phase B:MeOH-HPLC; Flow rate: 20 mL/min; Gradient: 30% B to 30% B in 12 min).

Peak 1 (Isomer-3_D2E1): R_{T} 5.24 min; afforded a white solid (45 mg)

Peak 2 (Isomer-4_D2E2): R_{T} 6.8 min; afforded a white solid (40 mg)

### Step 9: 2-[1-1-(2-cyanophenyl)-1-(1-methylimidazol-4-yl)propan-2-yl]-5-hydroxy-1-methyl-N-(1,2-oxazol-4-yl)-6-oxopyrimidine-4-carboxamide

To a solution of 2-[(1-1-(2-cyanophenyl)-1-(1-methylimidazol-4-yl)propan-2-yl]-5-methoxy-1-methyl-N-(1,2-oxazol-4-yl)-6-oxopyrimidine-4-carboxamide (0.040 g, 0.084 mmol) dissolved in DMF (2 ml) was added LiBr (0.147 g, 1.7 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated an off-white solid (0.016 g, 41% yield)

ESI-MS m/ [symbol] : 460.0 [M+H]⁺; 90% ee

¹H NMR (300 MHz, DMSO-d6): δ 13.46 (brs, 1H), 10.01 (s, 1H), 9.18 (s, 1H), 8.76 (s, 1H), 7.98 (d, J=8.0 Hz, 1H), δ 7.89 (d, J=1.9 Hz, 1H), 7.85 (dd, J=7.7, 1.3 Hz, 1H), 7.82-7.71 (m, 2H), 7.53 (t, J=7.6 Hz, 1H), 6.50 (d, J=7.9 Hz, 1H), 4.29-4.16 (m, 1H), 3.91 (s, 3H), 3.52 (s, 3H), 1.22 (d, J=6.6 Hz, 3H)

Isomer-2_D1E2: Isolated an off-white solid (0.010 g, 26% yield)

ESI-MS m/ [symbol] : 460.0 [M+H]⁺; >98% ee

¹H NMR (300 MHz, DMSO-d6): δ 13.58 (brs, 1H), 10.05 (s, 1H), 9.17 (s, 1H), 8.76 (s, 1H), 7.97 (d, J=8.0 Hz, 1H), 7.91-7.82 (m, 2H), 7.80-7.71 (m, 2H), 7.52 (t, J=7.6 Hz, 1H), 6.49 (d, J=7.7 Hz, 1H), 4.36-4.18 (m, 1H), 3.91 (s, 3H), 3.52 (s, 3H), 1.22 (d, J=6.6 Hz, 3H).

Isomer-3_D2E1: Isolated an off-white solid (0.009 g, 21% yield)

ESI-MS m/ [symbol] : 460.0 [M+H]⁺; >96% ee

¹H NMR (300 MHz, DMSO-d6): δ 13.70 (s, 1H), 9.75 (s, 1H), 9.24 (s, 1H), 8.82 (s, 1H), 8.26 (d, J=8.1 Hz, 1H), 8.02 (d, J=7.7 Hz, 1H), 7.97-7.87 (m, 2H), 7.68 (t, J=7.6 Hz, 1H), 7.54 (s, 1H), 6.40 (d, J=10.4 Hz, 1H), 4.23 (dd, J=10.7, 6.6 Hz, 1H), 3.73 (s, 3H), 3.42 (s, 3H), 1.09 (d, J=6.6 Hz, 3H).

Isomer-4_D2E2: Isolated an off-white solid (0.006 g, 14% yield)

ESI-MS m/ [symbol] : 460.0 [M+H]⁺; >98% ee

¹H NMR (300 MHz, DMSO-d6): δ 13.65 (s, 1H), 9.74 (s, 1H), 9.24 (s, 1H), 8.82 (s, 1H), 8.25 (d, J=8.0 Hz, 1H), 8.02 (d, J=7.7 Hz, 1H), 7.99-7.86 (m, 2H), 7.68 (t, J=7.6 Hz, 1H), 7.54 (s, 1H), 6.40 (d, J=10.4 Hz, 1H), 4.23 (dd, J=10.6, 6.5 Hz, 1H), 3.73 (s, 3H), 3.43 (s, 3H), 1.09 (d, J=6.6 Hz, 3H).

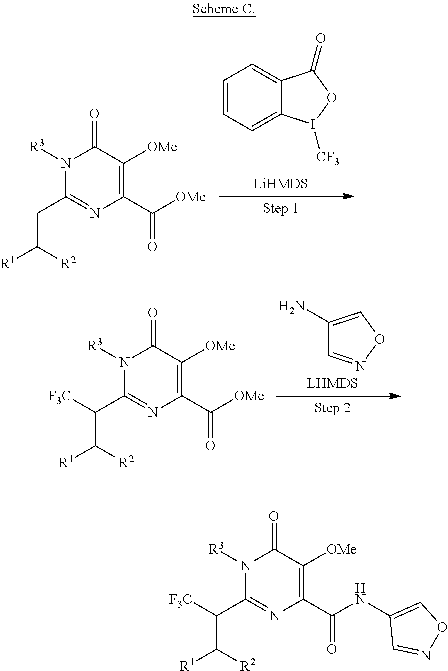

### Example 40

### Synthesis of 2-(3-(2-Cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide

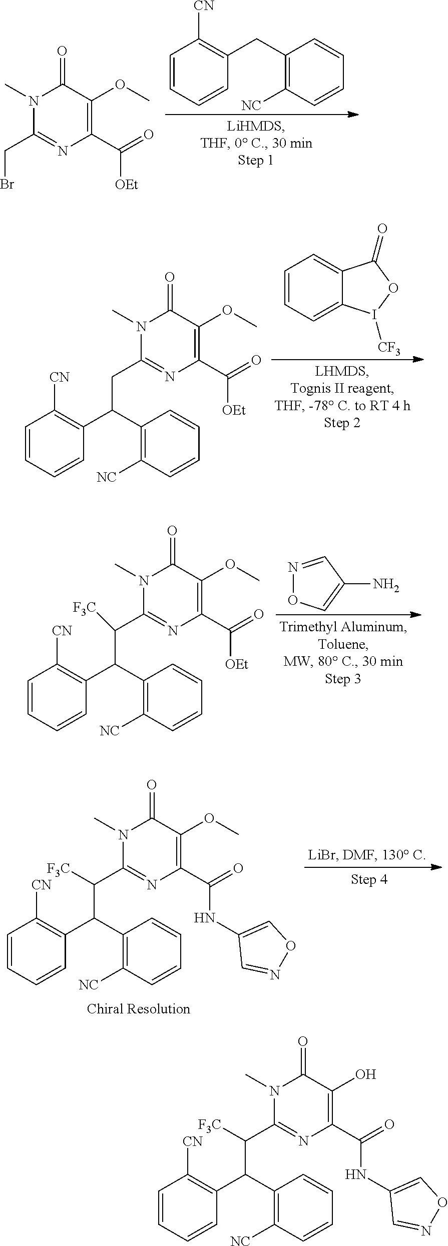

### Step 1: 2-((1-Methyl-1H-pyrazol-4-yl)methyl)benzonitrile

A mixture of 2-(Bromomethyl)benzonitrile (25.0 g, 127.51 mmol), (1-Methyl-1H-pyrazol-4-yl)boronic acid (16.05 g, 127.5 mmol) and sodium carbonate (27.0 g, 255.1 mmol) in a mixture of Toluene:Ethanol:water (7:3:4, 350 ml) was purged for 20 minutes with Argon gas. Pd(PPh₃)₄(7.36 g, 6.4 mmol) was added and purging was continued for another 10 minutes. The reaction mixture was heated in a sealed tube at 90° C. for 2 hours. After completion of reaction (monitored by TLC), the reaction mixture was filtered through Celite bed and filtrate was washed with EtOAc (3×500 ml). The combined organic layer was washed with brine (500 ml), dried over anhydrous sodium sulphate, and concentrated under reduced pressure. The crude compound was purified by using Combi-flash chromatography to obtain pure title compound (20 g, 79%).

¹H NMR (400 MHz, DMSO-d6): δ 3.76 (s, 3H), 3.95 (s, 2H), 7.28 (s, 1H), 7.41 (t, J=7.6 Hz, 1H), 7.47 (d, J=10.4 Hz, 2H), 7.65 (t, J=7.6 Hz, 1H), 7.78 (d, J=7.6 Hz, 1H).

### Step 2: Ethyl 2-(2-(2-cyanophenyl)-2-(1-methyl-1H-pyrazol-4-yl)ethyl)-5-methoxy-1-methyl-6-oxo-1,6-dihydropyrimidine-4-carboxylate

To a solution of 1M LiHMDS in THF (32.75 ml, 32.8 mmol) was cooled to −78° C. under Nitrogen atmosphere, 2-((1-Methyl-1H-pyrazol-4-yl)methyl)benzonitrile (5.1 g, 26.2 mmol) in DMF (25 ml) was added drop wise at −78° C. and reaction mixture was stirred at −78° C. for 30 minutes. Ethyl 2-(bromomethyl)-5-methoxy-1-methyl-6-oxo-1,6-dihydropyrimidine-4-carboxylate (4.0 g, 13.1 mmol) in DMF (20 ml) was added dropwise at −78° C. and stirred for 10 minutes. After completion of reaction (confirmed by TLC), water (100 ml) was added and reaction mixture was extracted with EtOAc (2×250 ml). The combined organic layer was washed with brine (200 ml), dried over anhydrous sodium sulphate, and concentrated under vacuum. The crude product was purified by RP column chromatography using acetonitrile and 0.1% Formic acid in water to give pure title compound (0.52 g, 9%) as light-yellow solid.

LCMS: m/ [symbol] 422.2 [M⁺+1].

¹H NMR (400 MHz, DMSO): δ 1.28 (t, J=7.2 Hz, 3H), 3.49-3.52 (m, 1H), 3.53 (s, 3H), 3.71-3.73 (m, 1H), 3.74 (s, 3H), 3.76 (s, 3H), 4.26 (q, J=7.2 Hz, 2H), 4.92-4.96 (m, 1H), 7.33-7.36 (m, 2H), 7.54 (s, 1H), 7.61 (t, J=7.6 Hz, 1H), 7.68-7.73 (m, 2H).

### Step 3: Ethyl 2-(3-(2-cyanophenyl)-1,1,1-trifluoro-3-(1-methyl-1H-pyrazol-4-yl)propan-2-yl)-5-methoxy-1-methyl-6-oxo-1,6-dihydropyrimidine-4-carboxylate

A solution of Ethyl 2-(2-(2-cyanophenyl)-2-(1-methyl-1H-pyrazol-4-yl)ethyl)-5-methoxy-1-methyl-6-oxo-1,6-dihydropyrimidine-4-carboxylate (1.3 g, 3.1 mmol) in THF (13 ml) was cooled to −78° C. under Nitrogen atmosphere. To it, 1M LiHMDS in THF (6.17 ml, 6.2 mmol) was added drop wise at −78° C. and stirred for 35 minutes. Togni Reagent II 60 wt. % (1.56 g, 4.9 mmol) was added at −78° C. and reaction mixture was stirred at −50° C. for 10 minutes followed by 0° C. for 10 minutes. After completion of reaction (monitored by TLC), water (50 ml) was added, and the reaction mixture was extracted with EtOAc (2×50 ml). The combined organic layer was washed with brine (50 ml), dried over anhydrous sodium sulphate, and concentrated under vacuum. The crude product was purified by RP column chromatography using acetonitrile and 0.1% Formic acid in water to give pure title compound (1.19 g, 78%) as yellow solid.

Isomer-1 (D1)_LCMS: m/ [symbol] : 490.2 [M⁺+1].

Isomer-2 (D2)_LCMS: m/ [symbol] : 490.2 [M⁺+1].

### Step 4: 2-(3-(2-cyanophenyl)-1,1,1-trifluoro-3-(1-methyl-1H-pyrazol-4-yl)propan-2-yl)-N-(isoxazol-4-yl)-5-methoxy-1-methyl-6-oxo-1,6-dihydropyrimidine-4-carboxamide

The solution of Ethyl 2-(3-(2-cyanophenyl)-1,1,1-trifluoro-3-(1-methyl-1H-pyrazol-4-yl)propan-2-yl)-5-methoxy-1-methyl-6-oxo-1,6-dihydropyrimidine-4-carboxylate (1.2 g, 2.5 mmol), Isooxazole amine (0.314 g, 3.7 mmol) in Toluene (12 ml) was cooled to 0° C. Trimethyl Aluminum solution (2.45 ml, 2M in Toluene, 4.9 mmol) was added at 0° C. The reaction mixture was heated at 80° C. for 1 hour under Microwave irradiation. After completion of reaction (confirmed by TLC), aqueous saturated sodium bicarbonate (20 ml) was added, and the reaction mixture was extracted with EtOAc (3×50 ml). The crude compound was purified by column chromatography to give racemic mixture of title compound (1.19 g, crude).

The diastereomeric mixture (1.19 g) was separated by using Reverse Phase-HPLC to get two separated diastereomers as D1 (0.150 and D2 (0.150 g).

Isomer-1 (D1)_LCMS: m/ [symbol] : 528.0 [M⁺+1].

Isomer-2 (D2)_LCMS: m/ [symbol] : 528.3 [M⁺+1].

Isomer-1 (D1): ¹H NMR (400 MHz, DMSO-d6): δ 3.70 (s, 3H), 3.76 (s, 3H), 3.78 (s, 3H), 5.33 ((d, J=11.2 Hz, 1H), 5.36-5.46 (m, 1H), 7.31 (t, J=7.2 Hz, 1H), 7.62-7.66 (m, 3H), 7.80 (s, 1H), 7.93 (d, J=8.0 Hz, 1H), 8.73 (s, 1H), 9.25 (s, 1H), 10.45 (s, 1H).

Isomer-2 (D2): ¹H NMR (400 MHz, DMSO-d6): δ 3.61 (s, 3H), 3.67 (s, 3H), 3.86 (s, 3H), 5.28 (bs, 2H), 7.29 (s, 1H), 7.47-7.53 (m, 2H), 7.77-7.84 (m, 2H), 8.30 (d, J=7.6 Hz, 1H), 8.75 (s, 1H), 9.34 (s, 1H), 10.72 (s, 1H).

### Chiral HPLC Method:

The diastereomers of title compound was resolved by Chiral SFC [D1: (CHIRALPAK IH (250*21) mm, 5 u; methanol in Liquid CO2+0.1% DEA)] and [D2: (CHIRALPAK IC(250*4.6) mm, IPA:ACN (50:50) in Liquid CO2+0.1% DEA] to furnish the enantiopure compounds.

Chiral HPLC: FR-1 (Isomer-1; D1E1): RT=11.25 (97%); FR-2 (Isomer-2; D1E2): RT=14.03 (99%); FR-3 (Isomer-3; D2E1): RT=4.44 (95%); FR-4 (Isomer-4; D2E2): RT=4.91 (100%).

### Step 5: 2-(3-(2-Cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide

To a solution of 2-(3-(2-Cyanophenyl)-1,1,1-trifluoro-3-(1-methyl-1H-pyrazol-4-yl)propan-2-yl)-N-(isoxazol-4-yl)-5-methoxy-1-methyl-6-oxo-1,6-dihydropyrimidine-4-carboxamide (0.06 g, 0.1 mmol) in DMF (0.6 ml) under nitrogen atmosphere, Lithium bromide (0.097 g, 1.1 mmol) was added at room temperature. The reaction mixture was heated at 130° C. for 1 hour under Microwave irradiation. After completion of reaction (confirmed by TLC), the reaction mixture was loaded on RP Gold column and purified using acetonitrile and 0.1% Formic acid in water to give pure title compound (0.028 g, 48%). Note: The above procedure for demethylation was followed for the remaining three isomers.

Isomer-1_(D1E1)_LCMS: m/z 514.1 [M⁺+1].

Isomer-2_(D1E2)_LCMS: m/z 514.6 [M⁺+1].

Isomer-3_(D2E1)_LCMS: m/z 514.2 [M⁺+1].

Isomer-4_(D2E2)_LCMS: m/z 514.5 [M⁺+1].

Isomer-1_D1E1: ¹H NMR (400 MHz, DMSO-d6): δ 3.69 (s, 3H), 3.80 (s, 3H), 5.35-5.47 (m, 1H), 5.50 (d, J=11.2 Hz, 1H), 7.28 (t, J=7.2 Hz, 1H), 7.64-7.67 (m, 3H), 7.83 (s, 1H), 7.94 (d, J=7.6 Hz, 1H), 8.88 (s, 1H), 9.30 (s, 1H), 10.34 (s, 1H), 11.57 (s, 1H).

Isomer-2_D1E2: ¹H NMR (400 MHz, DMSO-d6): δ 3.69 (s, 3H), 3.80 (s, 3H), 5.35-5.47 (m, 1H), 5.50 (d, J=11.2 Hz, 1H), 7.32-7.38 (m, 1H), 7.63-7.67 (m, 3H), 7.83 (s, 1H), 7.94 (d, J=6.8 Hz, 1H), 8.88 (s, 1H), 9.30 (s, 1H), 10.35 (s, 1H), 11.57 (s, 1H).

Isomer-3_D2E1: ¹H NMR (400 MHz, DMSO-d6): δ 3.63 (s, 3H), 3.67 (s, 3H), 5.29-5.35 (m, 1H), 5.50 (d, J=11.2 Hz, 1H), 7.36 (s, 1H), 7.51 (t, J=7.6 Hz, 1H), 7.57 (s, 1H), 7.80 (t, J=7.6 Hz, 1H), 7.88 (d, J=7.6 Hz, 1H), 8.21 (d, J=8 Hz, 1H), 8.82 (s, 1H), 9.36 (s, 1H), 10.26 (s, 1H), 11.30 (s, 1H).

Isomer-4_D2E2: ¹H NMR (400 MHz, DMSO-d6): δ 3.63 (s, 3H), 3.67 (s, 3H), 5.25-5.34 (m, 1H), 5.47 (d, J=11.2 Hz, 1H), 7.36 (s, 1H), 7.51 (t, J=7.2 Hz, 1H), 7.58 (s, 1H), 7.80 (t, J=7.6 Hz, 1H), 7.88 (d, J=7.6 Hz, 1H), 8.21 (d, J=7.2 Hz, 1H), 8.83 (s, 1H), 9.36 (s, 1H), 10.21 (s, 1H), 11.29 (s, 1H).

HPLC: FR-1 (Isomer-1; D1E1): R_{T}=4.60 (99%); FR-2 (Isomer-2; D1E2): R_{T}=4.60 (99%); FR-3 (Isomer-3; D2E1): R_{T}=4.79 (99%); FR-4 (Isomer-4; D2E2): R_{T}=4.79 (99%).

The following compounds in Table 4 were prepared according to the Scheme C methods described above.

TABLE 4

| ID | Structure | ¹H NMR | LCMS |
|---|---|---|---|
| Example 41 | 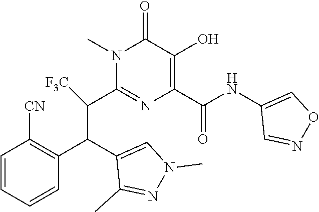 [SMILES: Cc1nn(C)cc1C(c1ccccc1C#N)C(c1nc(C(=O)Nc2cnoc2)c(O)c(=O)n1C)C(F)(F)F] | Isomer-1_ D1E1: ¹H NMR (400 MHz, DMSO-d6): δ 2.35 (s, 3H), 3.69 (s, 3H), 3.74 (s, 3H), 5.28-5.31 (m, 1H), 5.38 (d, J = 11.6 Hz, 1H), 7.28 (t, J = 7.6 Hz, 1H), 7.62-7.66 (m, 2H), 7.94 (d, J = 8.0 Hz, 1H), 7.98 (s, 1H), 8.86 (s, 1H), 9.32 (s, 1H), 10.20 (s, 1H), 11.52 (s, 1H). | Isomer- 1_(DIE1)_LCMS: m/z 528.3 [M⁺ + 1]. Isomer- 2_(D1E2)_LCMS m/z 528.5 [M⁺ + 1]. Isomer- 3_(D2E1)_LCMS: m/z 528.2 [M⁺ + 1]. Isomer- 4_(D2E2)_LCMS: m/z 528.2 [M⁺ + 1]. HPLC: FR-1 (Isomer- |
| | | Isomer-1_ D1E2: ¹H | 1; D1E1): R_{T} = 4.51 |
| | | NMR (400 MHz, | (100%); FR-2 (Isomer- |
| | | DMSO-d6): δ 2.35 (s, | 2; D1E2): R_{T} = 4.66 |
| | | 3H), 3.68 (s, 3H), 3.74 | (100%); FR-3 (Isomer- |
| | | (s, 3H), 5.28-5.31 (m, | 3; D2E1): R_{T} = 4.75 |
| | | 1H), 5.38 (d, J = 11.2 | (99%); FR-4 (Isomer-4; |
| | | Hz, 1H), 7.27 (t, J = 7.6 | D2E2): R_{T} = 4.75 |
| | | Hz, 1H), 7.63-7.65 (m, | (100%). |
| | | 2H), 7.93 (d, J = 8.4 | |
| | | Hz, 1H), 7.97 (s, 1H), | |
| | | 8.85 (s, 1H), 9.31 (s, | |
| | | 1H), 10.28 (s, 1H), | |
| | | 11.52 (s, 1H). | |
| | | Isomer-1_ D2E1: ¹H | |
| | | NMR (400 MHz, | |
| | | DMSO-d6): δ 2.03 (s, | |
| | | 3H), 3.64 (s, 3H), 3.70 | |
| | | (s, 3H), 5.25-5.27 (m, | |
| | | 1H), 5.42 (d, J = 11.2 | |
| | | Hz, 1H), 7.48 (t, J = 7.6 | |
| | | Hz, 1H), 7.78 (t, J = 7.6 | |
| | | Hz, 1H), 7.85 (d, J = | |
| | | 7.6 Hz, 1H), 7.93 (s, | |
| | | 1H), 8.17 (t, J = 8.0 Hz, | |
| | | 1H), 8.78 (s, 1H), 9.34 | |
| | | (s, 1H), 10.21 (s, 1H), | |
| | | 11.29 (s, 1H). | |
| | | Isomer-1_ D2E2: ¹H | |
| | | NMR (400 MHz, | |
| | | DMSO-d6): δ 2.03 (s, | |
| | | 3H), 3.64 (s, 3H), 3.69 | |
| | | (s, 3H), 5.24-5.26 (m, | |
| | | 1H), 5.42 (d, J = 11.2 | |
| | | Hz, 1H), 7.48 (t, J = 7.6 | |
| | | Hz, 1H), 7.77 (t, J = 7.6 | |
| | | Hz, 1H), 7.85 (d, J = | |
| | | 7.6 Hz, 1H), 7.92 (s, | |
| | | 1H), 8.18 (t, J = 8.0 Hz, | |
| | | 1H), 8.78 (s, 1H), 9.34 | |
| | | (s, 1H), 10.29 (s, 1H), | |
| | | 11.20 (s, 1H). | |
| Example 42 | 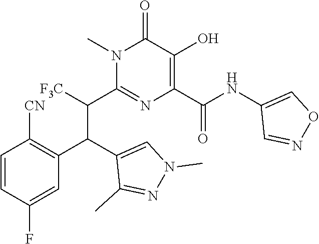 [SMILES: Cc1nn(C)cc1C(c1cc(F)ccc1C#N)C(c1nc(C(=O)Nc2cnoc2)c(O)c(=O)n1C)C(F)(F)F] | Isomer-1_ D1E1: ¹H NMR (400 MHz, DMSO-d6): δ 2.35 (s, 3H), 3.68 (s, 3H), 3.74 (s, 3H), 5.20-5.35 (m, 1H), 5.40 (d, J = 11.2 Hz, 1H), 7.17 (t, J = 8.4 Hz, 1H), 7.77 (t, J = 8.4 Hz, 1H), 7.93 (d, J = 10.0 Hz, 1H), 7.98 (s, 1H), 8.83 (s, 1H), 9.31 (s, 1H), 10.30 (s, 1H), 11.56 (s, 1H). Isomer-1_ D1E2: ¹H NMR (400 MHz, | Isomer- 1_(DIE1)_LCMS: m/z 546.3 [M⁺ + 1]. Isomer- 2_(D1E2)_LCMS m/z 546.3 [M⁺ + 1]. Isomer- 3_(D2E1)_LCMS: m/z 546.4 [M⁺ + 1]. Isomer- 4_(D2E2)_LCMS: m/z 546.2 [M⁺ + 1]. HPLC: FR-1 (Isomer- 1; D1E1): R_{T} = 4.61 (99%); FR-2 (Isomer-2; |
| | | DMSO-d6): δ 2.36 (s, | D1E2): R_{T} = 4.74 |
| | | 3H), 3.69 (s, 3H), 3.74 | (100%); FR-3 (Isomer- |
| | | (s, 3H), 5.20-5.35 (m, | 3; D2E1): R_{T} = 4.82 |
| | | 1H), 5.40 (d, J = 10.8 | (100%); FR-4 (Isomer- |
| | | Hz, 1H), 7.17 (t, J = 6.8 | 4; D2E2): RT = 4.18 |
| | | Hz, 1H), 7.77 (t, J = 8.0 | (100%). |
| | | Hz, 1H), 7.93 (d, J = | |
| | | 9.2 Hz, 1H), 7.98 (s, | |
| | | 1H), 8.83 (s, 1H), 9.30 | |
| | | (s, 1H), 10.34 (s, 1H), | |
| | | 11.55 (s, 1H). | |
| | | Isomer-1_ D2E1: ¹H | |
| | | NMR (400 MHz, | |
| | | DMSO-d6): δ 2.09 (s, | |
| | | 3H), 3.65 (s, 3H), 3.69 | |
| | | (s, 3H), 5.24-5.29 (m, | |
| | | 1H), 5.40 (d, J = 11.2 | |
| | | Hz, 1H), 7.40 (t, J = 8.4 | |
| | | Hz, 1H), 7.92 (s, 1H), | |
| | | 7.99 (t, J = 8.4 Hz, 1H), | |
| | | 8.16 (d, J = 10.4 Hz, | |
| | | 1H), 8.77 (s, 1H), 9.33 | |
| | | (s, 1H), 10.25 (s, 1H), | |
| | | 11.20 (s, 1H). | |
| | | Isomer-1_ D2E2: ¹H | |
| | | NMR (400 MHz, | |
| | | DMSO-d6): δ 2.05 (s, | |
| | | 3H), 3.65 (s, 3H), 3.68 | |
| | | (s, 3H), 5.24-5.29 (m, | |
| | | 1H), 5.40 (d, J = 11.2 | |
| | | Hz, 1H), 7.40 (t, J = 8.4 | |
| | | Hz, 1H), 7.92 (s, 1H), | |
| | | 7.98 (t, J = 8.8 Hz, 1H), | |
| | | 8.17 (d, J = 10.0 Hz, | |
| | | 1H), 8.77 (s, 1H), 9.33 | |
| | | (s, 1H), 10.45 (s, 1H), | |
| | | 11.21 (s, 1H). | |
| Example 131 | 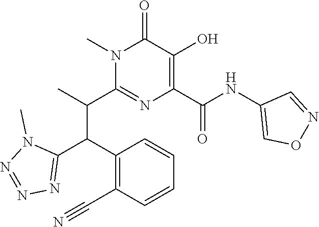 [SMILES: CC(c1nc(C(=O)Nc2cnoc2)c(O)c(=O)n1C)C(c1ccccc1C#N)c1nnnn1C] | Isomer-1_ D1E1: ¹H NMR (400 MHz, DMSO-d₆): δ 1.32 (d, J = 6.4 Hz, 3H), 3.55 (s, 3H), 4.29 (m,4H), 5.50 (d, J = 10.8 Hz, 1H), 7.34 (t, J = 7.6 Hz, 1H), 7.63-7.70 (m, 2H), 7.93 (d, J = 8 Hz, 1H), 8.84 (s, 1H), 9.31 (s, 1H), 10.66 (bs, 1H), 11.25 (s, 1H). Isomer-2_ D1E2: ¹H NMR (400 MHz, | Isomer-1 (D1E1)_LCMS: m/z 462.0 [M⁺ + 1]. Isomer- 2_(D1E2)_LCMS: m/z 462.0 [M⁺ + 1]. Isomer- 3_(D2E1)_LCMS: m/z 462.2 [M⁺ + 1]. Isomer- 4_(D2E2)_LCMS: m/z 462.0 [M⁺ + 1]. HPLC: FR-1 (Isomer- 1; D1E1): R_{T} = 4.43 |
| | | DMSO-d₆): δ 1.32 (d, | (99%); FR-2 (Isomer-2; |
| | | J = 6.4 Hz, 3H), 3.32 (s, | D1E2): R_{T} = 4.40 |
| | | 3H), 4.29 (s, 3H), 4.32 | (100%); FR-3 (Isomer- |
| | | (m, 1H), 5.50 (d, J = | 3; D2E1): R_{T} = 4.54 |
| | | 10.8 Hz, 1H), 7.34 (t, | (100%); FR-4 (Isomer- |
| | | J = 7.6 Hz, 1H), 7.63- | 4; D2E2): R_{T} = 4.54 |
| | | 7.70 (m, 2H), 7.93 (d, | (99%). |
| | | J = 8 Hz, 1H), 8.84 (s, | |
| | | 1H), 9.31 (s, 1H), 10.64 | |
| | | (bs, 1H), 11.25 (s, 1H). | |
| | | Isomer-3_ D2E1: ¹H | |
| | | NMR (400 MHz, | |
| | | DMSO-d₆): δ 1.17 (d, | |
| | | J = 8.0 Hz, 3H), 3.71 (s, | |
| | | 3H), 3.96 (s, 3H), 4.26 | |
| | | (m, 1H), 5.37 (d, J = 12 | |
| | | Hz, 1H), 7.58-7.62 (m, | |
| | | 1H) 7.82 (d, J = 4 Hz, | |
| | | 2H), 7.96 (d, J = 8 Hz, | |
| | | 1H), 8.81 (s, 1H), 9.30 | |
| | | (s, 1H), 10.49 (bs, 1H), | |
| | | 11.21 (bs, 1H). | |
| | | Isomer-4_ D2E2: ¹H | |
| | | NMR (400 MHz, | |
| | | DMSO-d₆): δ 1.17 (d, | |
| | | J = 6.8 Hz, 3H), 3.71 (s, | |
| | | 3H), 3.97 (s, 3H), 4.23 | |
| | | (m, 1H), 5.37 (d, J = 12 | |
| | | Hz, 1H), 7.58-7.62 (m, | |
| | | 1H), 7.82 (d, J = 4 Hz, | |
| | | 2H), 7.96 (d, J = 8 Hz, | |
| | | 1H), 8.81 (s, 1H), 9.29 | |
| | | (s, 1H), 10.52 (bs, 1H), | |
| | | 11.21 (bs, 1H). | |

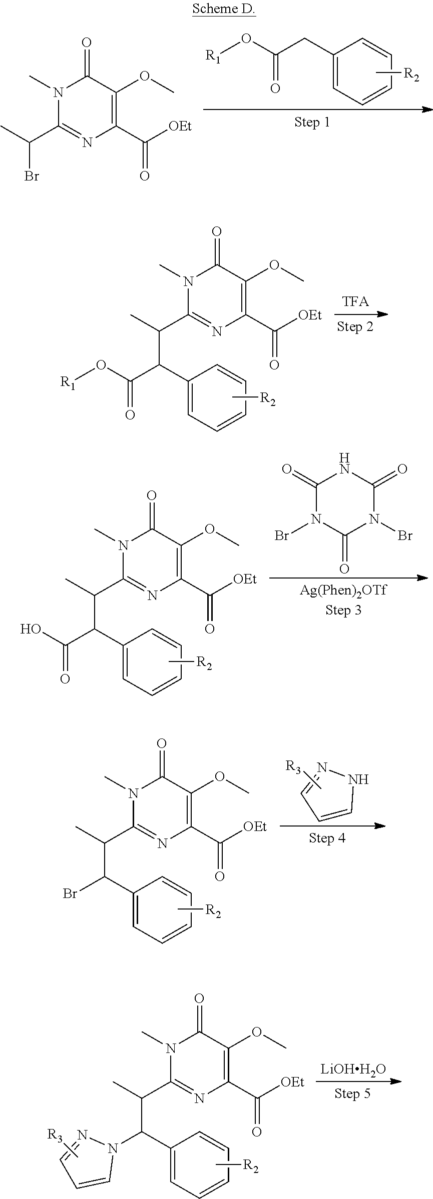

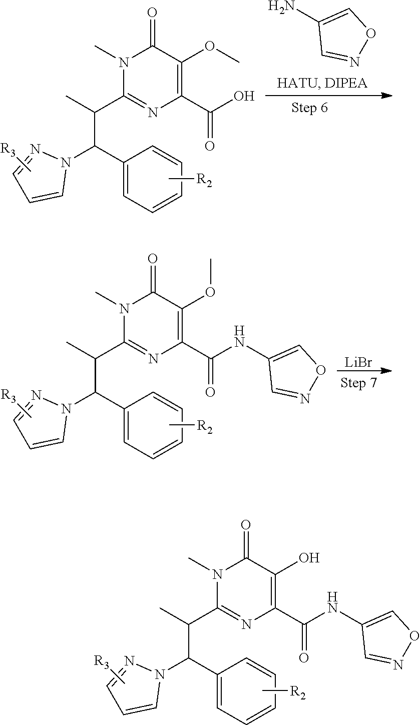

The following compounds in Table 5 were prepared according to the Scheme D methods described above.

TABLE 5

| | | | ESI-MS m/z |
|---|---|---|---|
| ID | Structure | ¹H NMR | [M + H] |
| Example 45 | 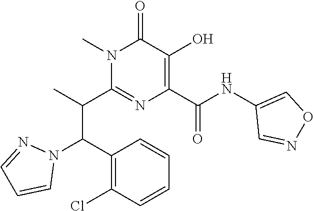 [SMILES: CC(c1nc(C(=O)Nc2cnoc2)c(O)c(=O)n1C)C(c1ccccc1Cl)n1cccn1] | | Isomer-1_D1E1: m/z 455.0 [M + H] >97% ee Isomer-2_D1E2: m/z 455.1 [M + H] >98% ee |
| Example 47 | 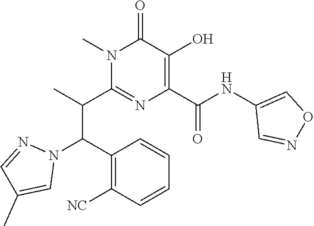 [SMILES: Cc1cnn(C(c2ccccc2C#N)C(C)c2nc(C(=O)Nc3cnoc3)c(O)c(=O)n2C)c1] | DIE1 ¹H NMR (400 MHz, DMSO- d₆) δ 11.22 (s, 1H), 10.58(s, 1H), 9.30 (s, 1H), 8.86 (s, 1H), 8.03 (d, 1H), 7.87 (s, 1H), 7.69-7.62 (m, 2H), 7.38-7.35 (m, 2H), 6.37 (d, 1H), 4.78-4.44 (m, 1H), 3.59(s, 3H), 2.04 (s, 3H), 1.24-1.23 (d, 3H). D1E2 ¹H NMR (400 MHz, DMSO- d6) δ 11.22 (s, 1H), 10.58 (s, 1H), 9.30 (s, 1H), 8.86 (s, | Isomer-1_D1E1: m/z 460.2 [M + H] >98% ee Isomer-2_D1E2: m/z 460.2 [M + H] >98% ee |
| | | 1H), 8.03 (d, 1H), 7.87 (s, | |
| | | 1H), 7.69-7.62 (m, 2H), | |
| | | 7.38-7.35 (m, 2H), 6.37 (d, | |
| | | 1H), 4.78-4.44 (m, 1H), | |
| | | 3.59(s, 3H), 2.04 | |
| Example 49 | 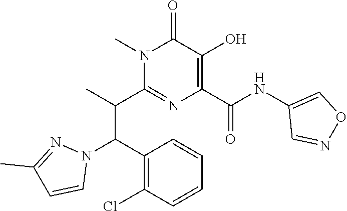 [SMILES: Cc1ccn(C(c2ccccc2Cl)C(C)c2nc(C(=O)Nc3cnoc3)c(O)c(=O)n2C)n1] | | Isomer-1_D1E1: m/z 469.1 [M + H] >98% ee Isomer-2_D1E2: m/z 469.1 [M + H] >98% ee |
| Example 54 | 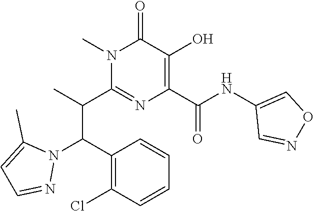 [SMILES: Cc1ccnn1C(c1ccccc1Cl)C(C)c1nc(C(=O)Nc2cnoc2)c(O)c(=O)n1C] | | Isomer-1_D1E1: m/z 469.2 [M + H] >98% ee Isomer-2_D1E2: m/z 469.1 [M + H] >98% ee |
| Example 132 | 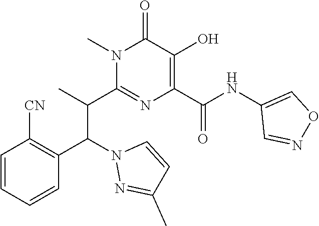 [SMILES: Cc1ccn(C(c2ccccc2C#N)C(C)c2nc(C(=O)Nc3cnoc3)c(O)c(=O)n2C)n1] | DIE1 ¹H NMR (400 MHz, DMSO- d₆) δ 11.22 (s, 1H), 10.58(s, 1H), 9.29 (s, 1H), 8.86 (s, 1H), 8.04-7.97 (m, 2H), 7.70-7.63 (m, 2H), 7.39-7.35 (m, 1H), 6.36-6.34 (d, 1H), 6.12 (s, 1H), 4.43-4.41 (m, 1H), 3.59(s, 3H), 2.18 (s, 3H), 1.22-1.21 (d, 3H). DIE2 1H NMR (400 MHz, DMSO- d₆) δ 11.22 (s, 1H), 10.58(s, 1H), 9.29 (s, 1H), 8.86 (s, | Isomer-1_D1E1: m/z 460.2 [M + H] >98% ee Isomer-2_D1E2: m/z 460.2 [M + H] >98% ee |
| | | 1H), 8.04-7.97 (m, 2H), | |
| | | 7.70-7.63 (m, 2H), 7.39-7.35 | |
| | | (m, 1H), 6.36 (d, 1H), 6.12 | |
| | | (s, 1H), 4.43-4.41 (m, 1H), | |
| | | 3.59(s, 3H), 2.18 (s, 3H), | |
| | | 1.22-1.21 (d, 3H). | |
| Example 133 | 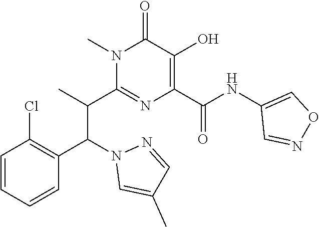 [SMILES: Cc1cnn(C(c2ccccc2Cl)C(C)c2nc(C(=O)Nc3cnoc3)c(O)c(=O)n2C)c1] | D1E1 ¹H NMR (400 MHz, DMSO- d₆) δ 11.17 (s, 1H), 10.56 (s, 1H), 9.34 (s, 1H), 8.95 (s, 1H), 7.89-7.87 (m, 1H), 7.80 (s, 1H), 7.35 (s, 1H), 7.29-7.24 (m, 2H), 7.18- 7.14 (m, 1H), 6.54 (d, 1H), 4.43-4.39 (m, 1H), 3.61 (s, 3H), 2.03 (s, 3H), 1.19 (d, 3H). D1E2 ¹H NMR (400 MHz, DMSO- d₆) δ 11.17 (s, 1H), 10.55 (s, | Isomer-1_D1E1: m/z 469.3 [M + H] >98% ee Isomer-2_D1E2: m/z 469.3 [M + H] >98% ee |
| | | 1H), 9.34 (s, 1H), 8.95 (s, | |
| | | 1H), 7.89-7.87 (m, 1H), | |
| | | 7.80 (s, 1H), 7.35 (s, 1H), | |
| | | 7.29-7.24 (m, 2H), 7.18- | |
| | | 7.14 (m, 1H), 6.54 (d, 1H), | |
| | | 4.44-4.39 (m, 1H), 3.61 (s, | |
| | | 3H), 2.03 (s, 3H), 1.19 (d, | |
| | | 3H). | |
| Example 134 | 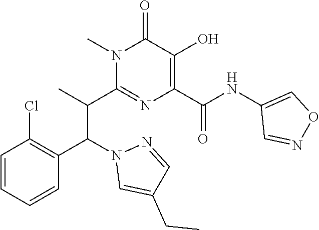 [SMILES: CCc1cnn(C(c2ccccc2Cl)C(C)c2nc(C(=O)Nc3cnoc3)c(O)c(=O)n2C)c1] | DIE1 ¹H NMR (400 MHz, DMSO- d₆) δ 11.17 (s, 1H), 10.55 (s, 1H), 9.34 (s, 1H), 8.95 (s, 1H), 7.91-7.90 (m, 1H), 7.81 (s, 1H), 7.40 (s, 1H), 7.29-7.25 (m, 2H), 7.18- 7.15 (m, 1H), 6.56 (d, 1H), 4.45-4.38 (m, 1H), 3.61 (s, 3H), 2.51-2.42 (m, 2H), 1.18-1.13 (m, 6H). D1E2 ¹H NMR (400 MHz, DMSO- d₆) δ 11.17 (s, 1H), 10.55 (s, | Isomer-1_D1E1: m/z 483.2 [M + H] >98% ee Isomer-2_D1E2: m/z 483.2 [M + H] >98% ee |
| | | 1H), 9.34 (s, 1H), 8.95 (s, | |
| | | 1H), 7.91-7.90 (m, 1H), | |
| | | 7.81 (s, 1H), 7.40 (s, 1H), | |
| | | 7.29-7.25 (m, 2H), 7.18- | |
| | | 7.17 (m, 1H), 6.56 (d, 1H), | |
| | | 4.43-4.41 (m, 1H), 3.61 (s, | |
| | | 3H), 2.51-2.44 (m, 2H), | |
| | | 1.18-1.13 (m, 6H). | |
| Example 135 | 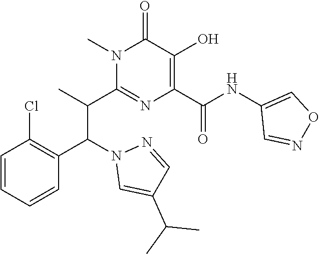 [SMILES: CC(C)c1cnn(C(c2ccccc2Cl)C(C)c2nc(C(=O)Nc3cnoc3)c(O)c(=O)n2C)c1] | DIE1 ¹H NMR (400 MHz, DMSO- d₆) δ 11.16 (s, 1H), 10.56 (s, 1H), 9.34 (s, 1H), 8.96 (s, 1H), 7.92 (d, 1H), 7.80 (s, 1H), 7.43 (s, 1H), 7.35 (s, 1H), 7.28 (d, 2H), 7.17 (t, 1H), 6.57 (d, 1H), 4.42- 4.38 (m, 1H), 3.61 (s, 3H), 2.84-2.79 (m, 1H), 1.18- 1.15 (m, 9H). DIE2 ¹H NMR (400 MHz, DMSO- d₆) δ 11.16 (s, 1H), 10.56 (s, 1H), 9.34 (s, 1H), 8.96 (s, | Isomer-1_D1E1: m/z 497.3 [M + H] >98% ee Isomer-2_D1E2: m/z 497.3 [M + H] >98% ee |
| | | 1H), 7.92 (d, 1H), 7.80 (s, | |
| | | 1H), 7.43 (s, 1H), 7.35 (s, | |
| | | 1H), 7.28 (d, 2H), 7.17 (t, | |
| | | 1H), 6.57 (d, 1H), 4.42- | |
| | | 4.38 (m, 1H), 3.61 (s, 3H), | |
| | | 2.84-2.78 (m, 1H), 1.19- | |
| | | 1.15 (m, 9H). | |
| Example 136 | 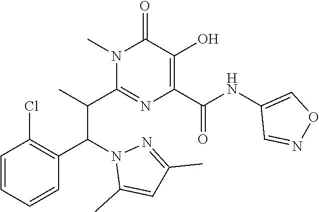 [SMILES: Cc1cc(C)n(C(c2ccccc2Cl)C(C)c2nc(C(=O)Nc3cnoc3)c(O)c(=O)n2C)n1] | DIE1 ¹H NMR (400 MHz, DMSO- d₆) δ 11.21 (s, 1H), 10.54 (s, 1H), 9.34 (s, 1H), 8.93 (s, 1H), 7.93 (d, 1H), 7.26 (d, 2H), 7.16 (t, 1H), 6.24 (d, 1H), 5.84 (s, 1H), 4.37- 4.33 (m, 1H), 3.54 (s, 3H), 2.52 (s, 3H), 2.14 (s, 3H), 1.20 (d, 3H). DIE2 ¹H NMR (400 MHz, DMSO- d₆) δ 11.21 (s, 1H), 10.54 (s, | Isomer-1_D1E1: m/z 483.2 [M + H] >98% ee Isomer-2_D1E2: m/z 483.2 [M + H] >98% ee |
| | | 1H), 9.34 (s, 1H), 8.93 (s, | |
| | | 1H), 7.93 (d, 1H), 7.26 (d, | |
| | | 2H), 7.16 (t, 1H), 6.24 (d, | |
| | | 1H), 5.84 (s, 1H), 4.37- | |
| | | 4.32 (m, 1H), 3.54 (s, 3H), | |
| | | 2.52 (s, 3H), 2.14 (s, 3H), | |
| | | 1.20 (d, 3H). | |
| Example 137 | 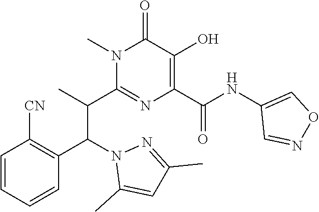 [SMILES: Cc1cc(C)n(C(c2ccccc2C#N)C(C)c2nc(C(=O)Nc3cnoc3)c(O)c(=O)n2C)n1] | DIE1 ¹H NMR (400 MHz, DMSO- d₆) δ 11.27 (s, 1H), 10.51 (s, 1H), 9.30 (s, 1H), 8.82 (s, 1H), 8.09 (d, 1H), 7.67- 7.64 (m, 2H), 7.39-7.35 (m, 1H), 6.10 (d, 1H), 5.88 (s, 1H), 4.43-4.36 (m, 1H), 3.53 (s, 3H), 2.54 (s, 3H), 2.16 (s, 3H), 1.23 (d, 3H). DIE2 ¹H NMR (400 MHz, DMSO- d₆) δ 11.27 (s, 1H), 10.51 (s, | Isomer-1_D1E1: m/z 474.2 [M + H] >98% ee Isomer-2_D1E2: m/z 474.2 [M + H] >98% ee |
| | | 1H), 9.30 (s, 1H), 8.82 (s, | |
| | | 1H), 8.09 (d, 1H), 7.67- | |
| | | 7.64 (m, 2H), 7.39-7.35 | |
| | | (m, 1H), 6.10 (d, 1H), 5.88 | |
| | | (s, 1H), 4.43-4.36 (m, 1H), | |
| | | 3.53 (s, 3H), 2.54 (s, 3H), | |
| | | 2.16 (s, 3H), 1.23 (d, 3H). | |
| Example 138 | 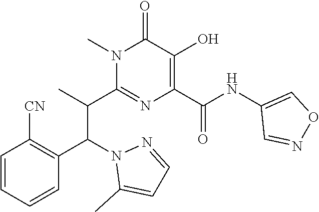 [SMILES: Cc1ccnn1C(c1ccccc1C#N)C(C)c1nc(C(=O)Nc2cnoc2)c(O)c(=O)n1C] | DIE1 ¹H NMR (400 MHz, DMSO- d₆) δ 11.28 (s, 1H), 10.56 (s, 1H), 9.30 (s, 1H), 8.83 (s, 1H), 8.12 (d, 1H), 7.69-7.63 (m, 2H), 7.51 (s, 1H), 7.39- 7.35 (m, 1H), 6.22 (d, 1H), 6.10 (s, 1H), 4.47-4.43 (m, 1H), 3.53 (s, 3H), 2.59 (s, 3H), 1.23 (d, 3H). D1E2 ¹H NMR (400 MHz, DMSO- d₆) δ 11.28 (s, 1H), 10.56 (s, | Isomer-1_D1E1: m/z 460.2 [M + H] >98% ee Isomer-2_D1E2: m/z 460.2 [M + H] >98% ee |
| | | 1H), 9.30 (s, 1H), 8.83 (s, | |
| | | 1H), 8.12 (d, 1H), 7.69-7.63 | |
| | | (m, 2H), 7.51 (s, 1H), 7.39- | |
| | | 7.35 (m, 1H), 6.22 (d, 1H), | |
| | | 6.10 (s, 1H), 4.47-4.43 (m, | |
| | | 1H), 3.53(s, 3H), 2.59 (s, | |
| | | 3H), 1.23 (d, 3H). | |
| Example 139 | 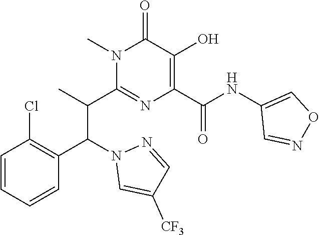 [SMILES: CC(c1nc(C(=O)Nc2cnoc2)c(O)c(=O)n1C)C(c1ccccc1Cl)n1cc(C(F)(F)F)cn1] | DIE1 ¹H NMR (300 MHz, DMSO- d₆) δ 11.12 (s, 1H), 10.51 (s, 1H), 9.34 (s, 1H), 8.95 (s, 1H), 8.73 (s, 1H), 8.04 (s, 1H), 7.92 (d, 1H), 7.40- 7.28 (m, 2H), 7.27-7.15 (m, 1H), 6.79 (d, 1H), 4.52- 4.44 (m, 1H), 3.63 (s, 3H), 1.19 (d, 3H). D1E2 ¹H NMR (300 MHz, DMSO- d₆) δ 11.12 (s, 1H), 10.52 (s, 1H), 9.34 (s, 1H), 8.95 (s, | Isomer-1_D1E1: m/z 523.2 [M + H] >98% ee Isomer-2_D1E2: m/z 523.2 [M + H] >98% ee |
| | | 1H), 8.73 (s, 1H), 8.04 (s, | |
| | | 1H), 7.92 (d, 1H), 7.40- | |
| | | 7.27 (m, 2H), 7.26-7.15 | |
| | | (m, 1H), 6.79 (d, 1H), 4.52- | |
| | | 4.44 (m, 1H), 3.63 (s, 3H), | |
| | | 1.19 (d, 3H). | |
| Example 140 | 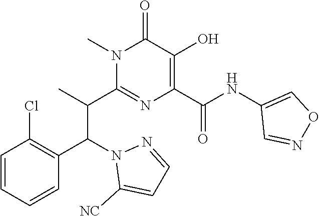 [SMILES: CC(c1nc(C(=O)Nc2cnoc2)c(O)c(=O)n1C)C(c1ccccc1Cl)n1nccc1C#N] | DIE1 ¹H NMR (400 MHz, DMSO- d₆) δ 11.16 (s, 1H), 10.43 (s, 1H), 9.31 (s, 1H), 8.85 (s, 1H), 7.94 (d, 1H), 7.82 (d, 1H), 7.44-7.16 (m, 4H), 6.64 (d, 1H), 4.47-4.44 (m, 1H), 3.56 (s, 3H), 1.25 (d, 3H). DIE2 ¹H NMR (400 MHz, DMSO- d₆) δ 11.16 (s, 1H), 10.45 (s, 1H), 9.31 (s, 1H), 8.85 (s, | Isomer-1_D1E1: m/z 480.0 [M + H] >98% ee Isomer-2_D1E2: m/z 480.0 [M + H] >98% ee |
| | | 1H), 7.94 (d, 1H), 7.82 (d, | |
| | | 1H), 7.44-7.11 (m, 4H), | |
| | | 6.64 (d, 1H), 4.49-4.45 (m, | |
| | | 1H), 3.56 (s, 3H), 1.25 (d, | |
| | | 3H). | |
| Example 141 | 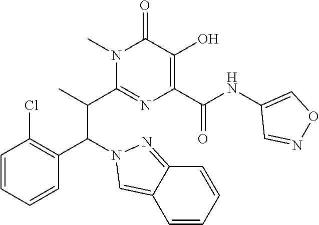 [SMILES: CC(c1nc(C(=O)Nc2cnoc2)c(O)c(=O)n1C)C(c1ccccc1Cl)n1cc2ccccc2n1] | DIE1 ¹H NMR (400 MHz, DMSO- d₆) δ 11.15 (s, 1H), 10.63 (s, 1H), 9.36 (s, 1H), 8.97 (s, 1H), 8.72 (d, 1H), 8.08- 8.06 (m, 1H), 7.80-7.78 (m, 1H), 7.66-7.64 (m, 1H), 7.33-7.24 (m, 3H), 7.21-7.17 (m, 1H), 7.09- 7.05 (m, 1H), 7.00 (d, 1H), 4.69-4.62 (m, 1H), 3.68 (s, 3H), 1.16 (d, 3H). D1E2 ¹H NMR (400 MHz, DMSO- | Isomer-1_D1E1: m/z 505.2 [M + H] >98% ee Isomer-2_D1E2: m/z 505.2 [M + H] >98% ee |
| | | d₆) δ 11.15 (s, 1H), 10.63 (s, | |
| | | 1H), 9.36 (s, 1H), 8.97 (s, | |
| | | 1H), 8.72 (d, 1H), 8.08- | |
| | | 8.06 (m, 1H), 7.80-7.78 | |
| | | (m, 1H), 7.66-7.64 (m, | |
| | | 1H), 7.33-7.24 (m, 3H), | |
| | | 7.21-7.17 (m, 1H), 7.09- | |
| | | 7.05 (m, 1H), 7.00 (d, 1H), | |
| | | 4.69-4.62 (m, 1H), 3.68 (s, | |
| | | 3H), 1.16 (d, 3H). | |
| Example 142 | 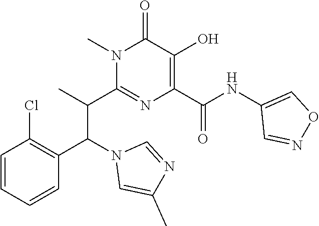 [SMILES: Cc1cn(C(c2ccccc2Cl)C(C)c2nc(C(=O)Nc3cnoc3)c(O)c(=O)n2C)cn1] | DIE1 ¹H NMR (400 MHz, DMSO- d₆) δ 11.12 (s, 1H), 10.53 (s, 1H), 9.32 (s, 1H), 8.92 (s, 1H), 7.90-7.85 (m, 2H), 7.32-7.17 (m, 4H), 6.53- 6.47 (m, 1H), 4.46-4.41 (m, 1H), 3.66 (s, 3H), 2.08 (s, 3H), 1.19 (d, 3H). D1E2 ¹H NMR (400 MHz, DMSO- d₆) δ 11.09 (s, 1H), 10.48 (s, 1H), 9.33 (s, 1H), 8.92 (s, 1H), 7.90-7.84 (m, 2H), | Isomer-1_D1E1: m/z 469.2 [M + H] >98% ee Isomer-2_D1E2: m/z 469.2 [M + H] >98% ee |
| | | 7.33-7.27 (m, 3H), 7.23- | |
| | | 7.18 (m, 1H), 6.52-6.47 | |
| | | (m, 1H), 4.46-4.41 (m, 1H), | |
| | | 3.66 (s, 3H), 2.08 (s, 3H), | |
| | | 1.19 (d, 3H). | |
| Example 143 | 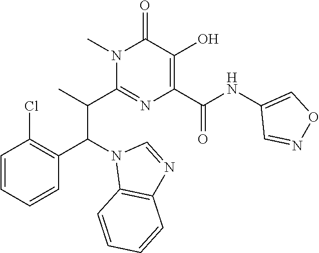 [SMILES: CC(c1nc(C(=O)Nc2cnoc2)c(O)c(=O)n1C)C(c1ccccc1Cl)n1cnc2ccccc21] | DIE1 ¹H NMR (300 MHz, DMSO- d₆) δ 11.23 (s, 1H), 10.72 (s, 1H), 9.38 (s, 1H), 9.00 (s, 1H), 8.83 (s, 1H), 8.24 (d, 1H), 8.02 (d, 1H), 7.67 (d, 1H), 7.47-7.06 (m, 5H), 6.82 (d, 1H), 4.87-4.51 (m, 1H), 3.70 (s, 3H), 1.24 (d, 3H). DIE2 ¹H NMR (300 MHz, DMSO- d₆) δ 11.23 (s, 1H), 10.72 (s, 1H), 9.38 (s, 1H), 9.00 (s, 1H), 8.82 (s, 1H), 8.24 (d, | Isomer-1_D1E1: m/z 505.1 [M + H] >98% ee Isomer-2_D1E2: m/z 505.1 [M + H] >98% ee |
| | | 1H), 8.02 (d, 1H), 7.67 (d, | |
| | | 1H), 7.44-7.11 (m, 5H), | |
| | | 6.82 (d, 1H), 4.72-4.66 (m, | |
| | | 1H), 3.70 (s, 3H), 1.24 (d, | |
| | | 3H). | |
| Example 144 | 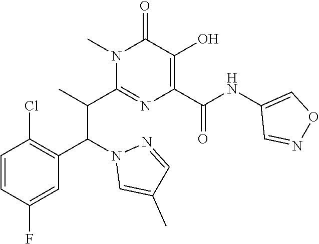 [SMILES: Cc1cnn(C(c2cc(F)ccc2Cl)C(C)c2nc(C(=O)Nc3cnoc3)c(O)c(=O)n2C)c1] | DIE1 ¹H NMR (300 MHz, DMSO- d₆) δ 11.17 (s, 1H), 10.52 (s, 1H), 9.34 (s, 1H), 8.93 (s, 1H), 7.83-7.77 (m, 2H), 7.38-7.33 (m, 2H), 7.09- 7.02 (m, 1H), 6.55 (dd, 1H), 4.43-4.33 (m, 1H), 3.63 (s, 3H), 2.05 (s, 3H), 1.16 (d, 3H). DIE2 ¹H NMR (300 MHz, DMSO- d₆) δ 11.17 (s, 1H), 10.52 (s, 1H), 9.34 (s, 1H), 8.93 (s, 1H), 7.83-7.77 (m, 2H), | Isomer-1_D1E1: m/z 487.1 [M + H] >98% ee Isomer-2_D1E2: m/z 487.1 [M + H] >98% ee |
| | | 7.38-7.33 (m, 2H), 7.09- | |
| | | 7.02 (m, 1H), 6.55 (dd, 1H), | |
| | | 4.43-4.33 (m, 1H), 3.63 (s, | |
| | | 3H), 2.05 (s, 3H), 1.16 (d, | |
| | | 3H). | |
| Example 145 | 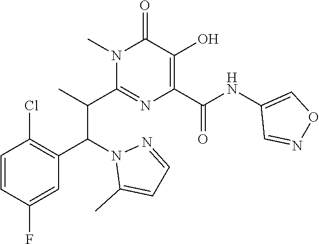 [SMILES: Cc1ccnn1C(c1cc(F)ccc1Cl)C(C)c1nc(C(=O)Nc2cnoc2)c(O)c(=O)n1C] | DIE1 ¹H NMR (300 MHz, DMSO- d₆) δ 11.23 (s, 1H), 10.55 (s, 1H), 9.35 (s, 1H), 8.93 (s, 1H), 7.80 (dd, 1H), 7.51 (d, 1H), 7.36 (dd, 1H), 7.11- 7.05 (m, 1H), 6.35 (d, 1H), 6.09 (dd, 1H), 4.40-4.34 (m, 1H), 3.57 (s, 3H), 2.59 (s, 3H), 1.17 (d, 3H). DIE2 ¹H NMR (300 MHz, DMSO- d₆) δ 11.23 (s, 1H), 10.56 (s, 1H), 9.35 (s, 1H), 8.93 (s, 1H), 7.80 (dd, 1H), 7.51 (d, | Isomer-1_D1E1: m/z 487.1 [M + H] >98% ee Isomer-2_D1E2: m/z 487.1 [M + H] >98% ee |
| | | 1H), 7.36 (dd, 1H), 7.11- | |
| | | 7.05 (m, 1H), 6.35 (d, 1H), | |
| | | 6.09 (dd, 1H), 4.40-4.34 | |
| | | (m, 1H), 3.57 (s, 3H), 2.59 | |
| | | (s, 3H), 1.17 (d, 3H). | |
| Example 146 | 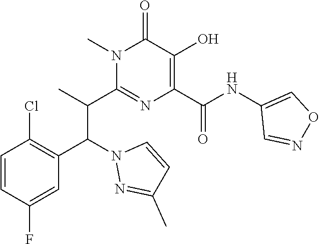 [SMILES: Cc1ccn(C(c2cc(F)ccc2Cl)C(C)c2nc(C(=O)Nc3cnoc3)c(O)c(=O)n2C)n1] | DIE1 ¹H NMR (300 MHz, DMSO- d6) δ 11.17 (s, 1H), 10.53 (s, 1H), 9.34 (s, 1H), 8.94 (s, 1H), 7.95 (d, 1H), 7.81 (dd, 1H), 7.36 (dd, 1H), 7.10- 7.04 (m, 1H), 6.55 (d, 1H), 6.11 (d, 1H), 4.39-4.33 (m, 1H), 3.64 (s, 3H), 2.19 (s, 3H), 1.15 (d, 3H). DIE2 ¹H NMR (300 MHz, DMSO- d6) δ 11.17 (s, 1H), 10.53 (s, 1H), 9.34 (s, 1H), 8.94 (s, 1H), 7.95 (d, 1H), 7.81 (dd, | Isomer-1_D1E1: m/z 487.1 [M + H] >98% ee Isomer-2_D1E2: m/z 487.1 [M + H] >98% ee |
| | | 1H), 7.36 (dd, 1H), 7.10- | |
| | | 7.04 (m, 1H), 6.55 (d, 1H), | |
| | | 6.11 (d, 1H), 4.39-4.33 (m, | |
| | | 1H), 3.64 (s, 3H), 2.19 (s, | |
| | | 3H), 1.15 (d, 3H). | |
| Example 147 | 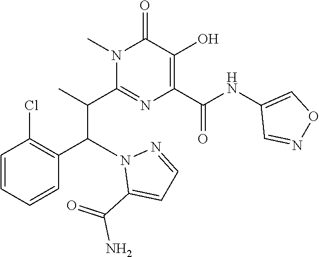 [SMILES: CC(c1nc(C(=O)Nc2cnoc2)c(O)c(=O)n1C)C(c1ccccc1Cl)n1nccc1C(N)=O] | DIE1 ¹H NMR (400 MHz, DMSO- d₆) δ 10.73 (s, 1H), 10.44 (s, 1H), 9.39 (s, 1H), 9.05 (s, 1H), 8.14 (d, 2H), 8.06 (d, 1H), 7.87 (s, 1H), 7.63 (d, 1H), 7.34 (t, 1H), 7.32- 7.25 (m, 2H), 6.92 (d, 1H), 4.40-4.39 (m, 1H), 3.47 (s, 3H), 1.02 (d, 3H). D1E2 ¹H NMR (400 MHz, DMSO- d₆) δ 10.73 (s, 1H), 10.45 (s, 1H), 9.39 (s, 1H), 9.05 (s, 1H), 8.24-8.11 (m, 2H), | Isomer-1_D1E1: m/z 498.0 [M + H] >98% ee Isomer-2_D1E2: m/z 498.0 [M + H] >98% ee |
| | | 8.06 (d, 1H), 7.87 (s, 1H), | |
| | | 7.63 (d, 1H), 7.35-7.27 (m, | |
| | | 3H), 6.92 (d, 1H), 4.40- | |
| | | 4.35 (m, 1H), 3.47 (s, 3H), | |
| | | 1.02 (d, 3H). | |

### Example 44

### Synthesis of 2-(1-(2-chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide

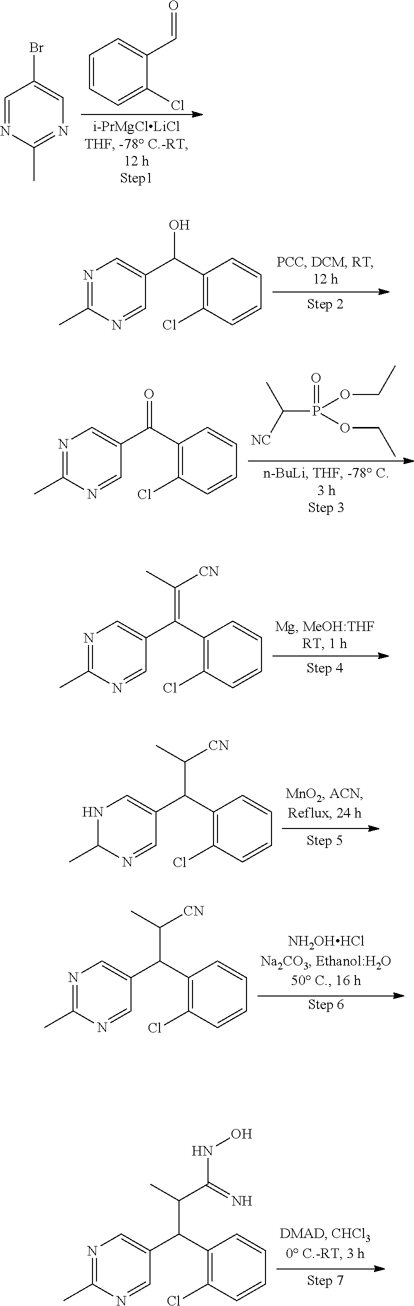

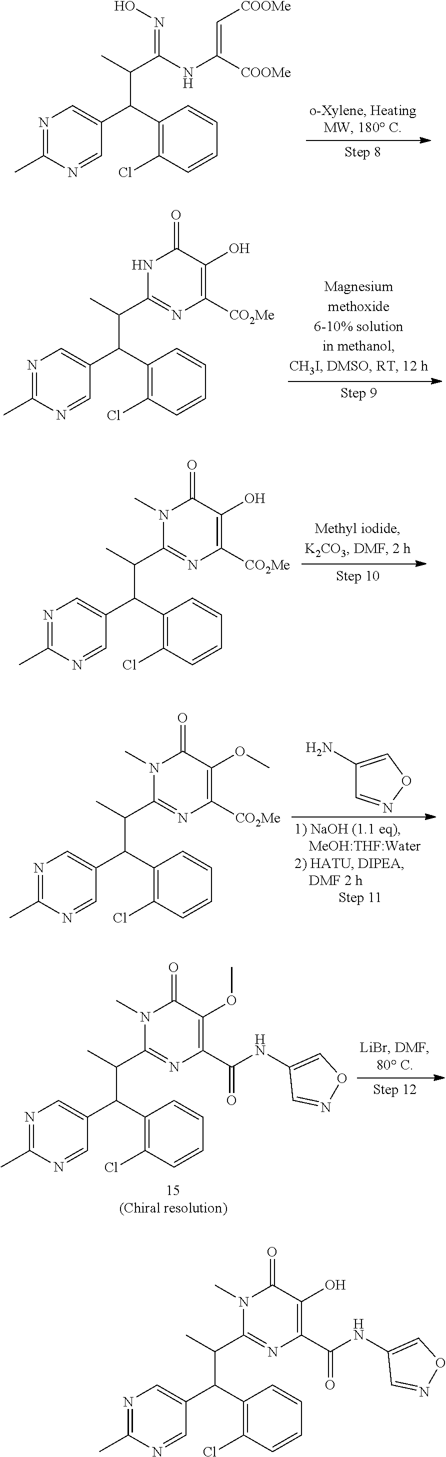

### Step 1: (2-Chlorophenyl)(2-methylpyrimidin-5-yl)methanol

A solution of 5-Bromo-2-methylpyrimidine (100 g, 578.0 mmol) in dry THF (500 ml) was added to a stirred solution of i-PrMgCl. LiCl (533.5 ml, 1.3 M in THF, 693.6 mmol) was added dropwise at −78° C. The reaction mixture was stirred at −78° C. for 1.5 hours. To this mixture, a solution of 2-Chlorobenzaldehyde (105.6 g, 751.4 mmol) in dry THF (500 ml) was added dropwise at −78° C. and the resulting reaction mixture was stirred at room temperature for 12 hours. After completion of reaction (monitored by the TLC), 10% aqueous NH4Cl solution (1000 ml) was added slowly. The organic layer was separated, and the aqueous layer was extracted with EtOAc (2×1000 ml). The combined organic layer was washed with brine (500 ml), dried over anhydrous sodium sulphate, filtered, and concentrated under reduced pressure. The crude compound was purified by using column-chromatography to give pure title compound (26 g, 19%).

LCMS: m/ [symbol] 5235.1 [M⁺+1].

¹H NMR (400 MHz, DMSO-d6): δ 2.59 (s, 3H), 6.03 (d, J=3.6 Hz, 1H), 6.40 (d, J=4.0 Hz, 1H), 7.32-7.36 (m, 1H), 7.42-7.46 (m, 2H), 7.79 (dd, J=7.6 Hz and 1.6 Hz, 1H), 8.60 (s, 2H).

### Step 2: (2-Chlorophenyl)(2-methylpyrimidin-5-yl)methanone

Pyridinium chlorochromate (50.51 g, 234.4 mmol) was added portion wise to a solution of (2-Chlorophenyl)(2-methylpyrimidin-5-yl)methanol (50 g, 213.1 mmol) in dry DCM (500 ml) under Nitrogen atmosphere at room temperature. The resulting reaction mixture was stirred for 12 hours at room temperature. After completion of reaction (monitored by TLC), the reaction mixture was filtered, through a Celite pad and washed with EtOAc (3×100 ml). The filtrate was concentrated under reduced pressure. The crude product was purified by using column-chromatography (n-Hexanes:EtOAc) to give pure title compound (30 g, 60%).

¹H NMR (400 MHz, DMSO-d6): δ 2.75 (s, 3H), 7.54-7.58 (m, 1H), 7.63-7.67 (m, 3H), 8.96 (s, 2H).

### Step 3: (E & Z)-3-(2-chlorophenyl)-2-methyl-3-(2-methylpyrimidin-5-yl)acrylonitrile

Diethyl (1-cyanoethyl)phosphonate in THF (90 ml) was added dropwise to a stirred solution of n-BuLi (2.3 M in n-Hexanes) (47.5 ml, 109.6 mmol) at −78° C. The resulting reaction mixture was stirred at −78° C. for 1 hour. To this mixture, (2-Chlorophenyl)(2-methylpyrimidin-5-yl)methanone (17 g, 73.1 mmol) in THF (80 ml) was added dropwise at −78° C. and the resulting reaction mixture was stirred at room temperature for 3 hours. After completion of the reaction (monitored by the TLC), 10% NH₄Cl solution in water (150 ml) was added slowly. The organic layer was separated, and the aqueous layer was extracted with EtOAc (2×250 ml). The combined organic layer was washed with brine (150 ml), dried over anhydrous sodium sulphate, filtered, and concentrated under reduced pressure. The crude product was purified by using column-chromatography (n-Hexanes:EtOAc) to give the title compound (9.6 g, 48%) as a mixture of E and Z isomers.

LCMS: m/ [symbol] 270.2 [M⁺+1]

¹H NMR (400 MHz, DMSO-d6): δ 1.91 (s, 3H), 2.17 (s, 3H), 2.65 (s, 3H), 2.66 (s, 3H), 7.50-7.64 (m, 8H), 8.61 (s, 2H), 8.70 (s, 2H).

### Step 4: 3-(2-Chlorophenyl)-2-methyl-3-(2-methyl-1,2-dihydropyrimidin-5-yl)propanenitrile

To a solution of (E & Z)-3-(2-chlorophenyl)-2-methyl-3-(2-methylpyrimidin-5-yl)acrylonitrile (10 g, 37.1 mmol), dry THF (100 ml) and MeOH (100 ml) was added Magnesium metal (9.01 g, 370.7 mmol) and NH₄Cl (0.982 g, 18.5 mmol) at room temperature under Nitrogen atmosphere. The resulting reaction mixture was stirred for 1 hour. After completion of reaction (monitored by TLC), the reaction mixture was filtered, through Celite pad and washed with EtOAc (2×50 ml) and filtrate was then concentrated under reduced pressure. The residue was taken in water (50 ml) and extracted with EtOAc (2×200 ml). The combined organic layer was washed with brine (100 ml), dried over anhydrous sodium sulphate, filtered, and concentrated under reduced pressure to obtain the crude title compound (10.4 g). The crude material was used in the next step without further purification.

LCMS: m/ [symbol] =273.2 [M+]

### Step 5: 3-(2-Chlorophenyl)-2-methyl-3-(2-methylpyrimidin-5-yl)propanenitrile

To a solution of 3-(2-Chlorophenyl)-2-methyl-3-(2-methyl-1,2-dihydropyrimidin-5-yl)propanenitrile (19.6 g, 71.56 mmol) in acetonitrile (196 ml), MnO₂ (9.33 g, 107.4 mmol) was added at room temperature under an atmosphere of Nitrogen. The resulting reaction mixture was refluxed for 24 hours. After completion of reaction (monitored by TLC), the reaction mixture was filtered, through Celite pad and Celite was washed with EtOAc (3×100 ml). The filtrate was concentrated under reduced pressure to afford crude title compound. The crude product was purified by using column-chromatography (n-Hexanes:EtOAc) to give pure title compound (10.1 g, 51%, two steps) as a mixture of diastereomers.

Isomer-1 (D1)_LCMS: m/ [symbol] 272.3 [M⁺+1]

Isomer-1 (D2)_LCMS: m/ [symbol] 272.3 [M⁺+1]

¹H NMR (400 MHz, DMSO-d6): 1.20-1.26 (m, 6H), 2.58 (s, 3H), 2.60 (s, 3H), 4.12-4.20 (m, 2H), 4.64 (d, J=11.6 Hz, 2H), 7.32-7.38 (m, 2H), 7.43-7.50 (m, 4H), 7.75 (d, J=8.0 Hz, 1H), 7.90 (d, J=8.0 Hz, 1H), 8.72 (s, 2H), 8.78 (s, 2H).

### Step 6: 3-(2-Chlorophenyl)-N-hydroxy-2-methyl-3-(2-methylpyrimidin-5-yl)propanimidamide

A mixture of 3-(2-Chlorophenyl)-2-methyl-3-(2-methylpyrimidin-5-yl)propanenitrile (1 g, 3.67 mmol), Hydroxylamine hydrochloride (0.384 g, 5.51 mmol) in Ethanol (10 ml) was added Na₂CO₃ (0.292 g, 2.75 mmol) and reaction mixture was heated to 50° C. for 16 hours. After completion of reaction (confirmed by TLC), the reaction mixture was concentrated, diluted with water (20 ml) and extracted with EtOAc (2×30 ml). The combined organic layer was dried over anhydrous sodium sulphate, filtered, and concentrated under reduced pressure to obtain crude title compound (2 g). The crude product was used in the next step without further purification.

Isomer-1 (D1)_LCMS: m/ [symbol] 304.9 [M⁺+1]

Isomer-1 (D2)_LCMS: m/ [symbol] 304.9 [M⁺+1]

### Step 7: Dimethyl 2-((E & Z)-3-(2-chlorophenyl)-N′-hydroxy-2-methyl-3-(2-methylpyrimidin-5-yl) propanimidamido) maleate

The solution of 3-(2-Chlorophenyl)-N-hydroxy-2-methyl-3-(2-methylpyrimidin-5-yl)propanimidamide (5.4 g crude, 17.71 mmol) in Chloroform (54 ml) was cooled to 0° C. To it, Dimethyl acetylenedicarboxylate (3.77 g, 26.6 mmol) was added dropwise and reaction mixture was stirred at room temperature for 3 hours. After completion of reaction (confirmed by TLC), the reaction mixture was concentrated and crude product was purified by silica gel column chromatography (n-Hexanes:EtOAc) to obtain pure title compound (1.6 g, 20%, two steps).

Isomer-1 (D1)_LCMS: m/ [symbol] 447.3 [M⁺+1]

Isomer-1 (D2)_LCMS: m/ [symbol] 447.3 [M⁺+1]

### Step 8: Methyl 2-(1-(2-chlorophenyl)-1-(2-methylpyrimidin-5-yl)propan-2-yl)-5-hydroxy-6-oxo-1,6-dihydropyrimidine-4-carboxylate

Dimethyl 2-((E & Z)-3-(2-chlorophenyl)-N′-hydroxy-2-methyl-3-(2-methylpyrimidin-5-yl)propanimidamido)maleate (3.4 g, 7.6 mmol) was dissolved in o-Xylene (34 ml) and heated at 180° C. in the microwave for 1 hour. After completion of reaction (confirmed by TLC), the reaction mixture was concentrated. The crude residue was loaded on Celite and purified by RP Gold column chromatography using acetonitrile and 0.1% Formic acid in water to give pure diastereomer-1 (0.520 g) and diastereomer-2 (0.300 g) as solid (Total 0.820 g, 26%).

Isomer-1 (D1)_LCMS: m/ [symbol] 415.3 [M⁺+1]

Isomer-2 (D2)_LCMS: m/ [symbol] 415.3 [M⁺+1]

Isomer-1 (D1)_¹H NMR (400 MHz, DMSO-d6): δ 1.09 (d, J=6.4 Hz, 3H), 2.57 (s, 3H), 3.73-3.79 (m, 4H), 4.85 (d, J=12.0 Hz, 1H), 7.14-7.17 (m, 1H), 7.29-7.36 (m, 2H), 7.76 (d, J=7.2 Hz, 1H), 8.70 (s, 2H), 10.17 (bs, 1H), 12.93 (s, 1H).

Isomer-2 (D2)_¹H NMR (400 MHz, DMSO-d6): δ 1.14 (d, J=6.4 Hz, 3H), 2.50 (s, 3H), 3.78-3.85 (m, 4H), 4.79 (d, J=12.0 Hz, 1H), 7.29-7.36 (m, 1H), 7.46-7.47 (m, 2H), 7.73 (d, J=7.2 Hz, 1H), 8.52 (s, 2H), 10.25 (bs, 1H), 12.89 (s, 1H).

### Step 9: Methyl 2-(1-(2-chlorophenyl)-1-(2-methylpyrimidin-5-yl)propan-2-yl)-5-hydroxy-1-methyl-6-oxo-1,6-dihydropyrimidine-4-carboxylate

A solution of Methyl 2-(1-(2-chlorophenyl)-1-(2-methylpyrimidin-5-yl)propan-2-yl)-5-hydroxy-6-oxo-1,6-dihydropyrimidine-4-carboxylate (0.420 g, 1.0 mmol) in DMSO (4.2 ml) was cooled to 0° C. To this, Magnesium methoxide solution (3.27 ml, 6 to 10% in methanol, 3.0 mmol) was added dropwise at 0° C. The reaction mixture was stirred at room temperature for 1 hour. The reaction mixture was concentrated to remove excess methanol, cooled to 0° C. and Methyl iodide (0.31 ml, 5.1 mmol) was added dropwise. The reaction mixture was stirred at room temperature for 16 hours. After completion of reaction (confirmed by TLC), the reaction mixture was cooled and quenched by dropwise addition of 1N HCl (1 ml). The product was extracted with EtOAc (2×30 ml). The combined organic layer was dried over anhydrous sodium sulphate, filtered, and concentrated under reduced pressure. The crude compound was purified by RP Gold column chromatography using acetonitrile and 0.1% Formic acid in water to give pure title compound (0.071 g, 16%) as a solid.

The same process was performed with Diastereomer-2 of Methyl 2-(1-(2-chlorophenyl)-1-(2-methylpyrimidin-5-yl)propan-2-yl)-5-hydroxy-6-oxo-1,6-dihydropyrimidine-4-carboxylate (270 mg).

Isolated product for Diastereomer-2 was (0.073 g, 26%).

Isomer-1 (D1)_LCMS: m/ [symbol] 429.3 [M⁺+1]

Isomer-2 (D2)_LCMS: m/ [symbol] 429.2 [M⁺+1]

Isomer-1 (D1)_¹H NMR (400 MHz, DMSO-d6): δ 1.08 (d, J=6.4 Hz, 3H), 2.57 (s, 3H), 3.70 (s, 3H), 3.76 (s, 3H), 4.21-4.30 (m, 1H), 4.97 (d, J=11.2 Hz, 1H), 7.09-7.11 (m, 1H), 7.20-7.29 (m, 2H), 7.68 (d, J=7.6 Hz, 1H), 8.87 (s, 2H), 10.11 (s, 1H).

Isomer-2 (D2)_¹H NMR (400 MHz, DMSO-d6): δ 1.12 (d, J=6.4 Hz, 3H), 2.45 (s, 3H), 3.67 (s, 3H), 3.90 (s, 3H), 4.22-4.26 (m, 1H), 5.08 (d, J=11.2 Hz, 1H), 7.29-7.31 (m, 1H), 7.45-7.49 (m, 2H), 8.04-8.14 (m, 1H), 8.68 (s, 2H), 10.25 (bs, 1H).

### Step 10: Methyl 2-(1-(2-chlorophenyl)-1-(2-methylpyrimidin-5-yl)propan-2-yl)-5-methoxy-1-methyl-6-oxo-1,6-dihydropyrimidine-4-carboxylate

To a stirred solution of Methyl 2-(1-(2-chlorophenyl)-1-(2-methylpyrimidin-5-yl)propan-2-yl)-5-hydroxy-1-methyl-6-oxo-1,6-dihydropyrimidine-4-carboxylate (0.070 g, 0.2 mmol) in DMF (0.7 ml) was added Cesium carbonate (0.106 g, 0.3 mmol) and reaction mixture was stirred at room temperature for 20 minutes. To this suspension, Methyl iodide (0.02 ml, 0.3 mmol) was added and reaction mixture was stirred at room temperature for 2 hours. After completion of reaction (monitor by TLC), the reaction mixture was diluted with water (20 ml) and aqueous layer was extracted with EtOAc (2×20 ml). The combined organic layer was washed with brine (20 ml), dried over anhydrous sodium sulphate, filtered, and concentrated under reduced pressure. The crude compound was purified by Combi-flash column chromatography to give pure title compound (0.051 g, 70%).

The same process was performed with Diastereomer-2 of Methyl 2-(1-(2-chlorophenyl)-1-(2-methylpyrimidin-5-yl)propan-2-yl)-5-hydroxy-1-methyl-6-oxo-1,6-dihydropyrimidine-4-carboxylate (70 mg). The Isolated product for Diastereomer-2 was (0.051 g, 70%).

Isomer-1 (D1)_LCMS: m/ [symbol] 443.6 [M⁺+1]

Isomer-2 (D2)_LCMS: m/ [symbol] 443.5 [M⁺+1]

Isomer-1 (D1)_¹H NMR (400 MHz, DMSO-d6): δ 1.10 (d, J=6.4 Hz, 3H), 2.57 (s, 3H), 3.69 (s, 3H), 3.71 (s, 3H), 3.78 (s, 3H), 4.30-4.35 (m, 1H), 4.95 (d, J=11.2 Hz, 1H), 7.12 (t, J=6.8 Hz, 1H), 7.23-7.31 (m, 2H), 7.69 (d, J=7.6 Hz, 1H), 8.88 (s, 2H).

Isomer-2 (D2)_¹H NMR (400 MHz, DMSO-d6): δ 1.13 (d, J=6.4 Hz, 3H), 2.46 (s, 3H), 3.60 (s, 3H), 3.76 (s, 3H), 3.89 (s, 3H), 4.27-4.32 (m, 1H), 5.03 (d, J=11.2 Hz, 1H), 7.30 (t, J=7.6 Hz, 1H), 7.44-7.48 (m, 2H), 8.08 (d, J=7.2 Hz, 1H), 8.64 (s, 2H).

### Step 11: 2-(1-(2-Chlorophenyl)-1-(2-methylpyrimidin-5-yl)propan-2-yl)-N-(isoxazol-4-yl)-5-methoxy-1-methyl-6-oxo-1,6-dihydropyrimidine-4-carboxamide

To a stirred solution of Methyl 2-(1-(2-chlorophenyl)-1-(2-methylpyrimidin-5-yl)propan-2-yl)-5-methoxy-1-methyl-6-oxo-1,6-dihydropyrimidine-4-carboxylate (0.050 g, 0.1 mmol) in methanol:THF:water (1:1:1, 1.5 ml) was added sodium hydroxide (0.0049 g, 0.1 mmol) at room temperature. The reaction mixture was stirred at room temperature for 1 hour. After completion of reaction (confirmed by TLC), the reaction mixture was concentrated under reduced pressure to give crude residue. The crude compound was triturated with Dichloromethane (3×5 ml) and dried under high vacuum to afford sodium 2-(1-(2-chlorophenyl)-1-(2-methylpyrimidin-5-yl)propan-2-yl)-5-methoxy-1-methyl-6-oxo-1,6-dihydropyrimidine-4-carboxylate (0.053 g). The crude compound was used in the next step without further purification.

To a stirred solution of sodium 2-(1-(2-chlorophenyl)-1-(2-methylpyrimidin-5-yl)propan-2-yl)-5-methoxy-1-methyl-6-oxo-1,6-dihydropyrimidine-4-carboxylate (0.053 g, 0.1 mmol) in DMF (0.5 ml) was added HATU (0.067 g, 0.2 mmol), and Isoxazol-4-amine (0.012 g, 0.2 mmol) at room temperature. The reaction mixture was stirred at room temperature for 30 minutes. DIPEA (0.06 ml, 0.351 mmol) was added at room temperature and the reaction mixture was stirred for 2 hours. After completion of reaction (monitor by TLC), the reaction mixture was diluted with water (10 ml) and aqueous layer was extracted with EtOAc (2×20 ml). The combined organic layer was washed with brine (20 ml), dried over anhydrous sodium sulphate, filtered, and concentrated under reduced pressure. The crude compound was purified by Combi-flash column chromatography to give pure title compound (0.044 g, 80%).

The same process was performed with another Diastereomer-1 of Methyl 2-(1-(2-chlorophenyl)-1-(2-methylpyrimidin-5-yl)propan-2-yl)-5-methoxy-1-methyl-6-oxo-1,6-dihydropyrimidine-4-carboxylate (60 mg). Isolated product for Diastereomer-1 was (0.040 g, 57%) as a solid.

Isomer-1 (D1)_LCMS: m/ [symbol] 495.3 [M⁺+1]

Isomer-2 (D2)_LCMS: m/ [symbol] 495.3 [M⁺+1]

Isomer-1 (D1)_¹H NMR (400 MHz, DMSO-d6): δ 1.17 (d, J=6.8 Hz, 3H), 2.60 (s, 3H), 3.71 (s, 3H), 3.75 (s, 3H), 4.37-4.42 (m, 1H), 5.24 (d, J=11.6 Hz, 1H), 7.10 (t, J=6.8 Hz, 1H), 7.25-7.27 (m, 2H), 7.76 (d, J=7.6 Hz, 1H), 8.92 (s, 2H), 9.06 (s, 1H), 9.30 (s, 1H), 10.25 (s, 1H).

Isomer-2 (D2)_¹H NMR (400 MHz, DMSO-d6): δ 1.19 (d, J=6.4 Hz, 3H), 2.46 (s, 3H), 3.60 (s, 3H), 3.78 (s, 3H), 4.31-4.35 (m, 1H), 5.05 (d, J=11.2 Hz, 1H), 7.31 (t, J=6.8 Hz, 1H), 7.45-7.48 (m, 2H), 8.08 (d, J=7.2 Hz, 1H), 8.62 (s, 2H), 9.06 (s, 1H), 9.32 (s, 1H), 10.63 (s, 1H).

### Chiral HPLC Method:

The diastereomers of title compound was resolved by Chiral SFC [D1: (CHIRALPAK IB-N(250*21) mm, 5 u; MeOH:IPA (50:50) in Hexanes+0.1% DEA)] and [D2: (CHIRALPAK IB-N(250*21) mm, 5 u; IPA in Hexanes+0.1% DEA)] to furnish the enantiopure compounds.

Chiral HPLC: FR-1 (Isomer-1; D1E1): RT=10.51; FR-2 (Isomer-2; D1E2): RT=12.02; FR-3 (Isomer-3; D2E1): RT=14.13; FR-4 (Isomer-4; D2E2): RT=16.86.

### Step 12: 2-(1-(2-Chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide

To a solution of 2-(1-(2-chlorophenyl)-1-(2-methylpyrimidin-5-yl)propan-2-yl)-N-(isoxazol-4-yl)-5-methoxy-1-methyl-6-oxo-1,6-dihydropyrimidine-4-carboxamide (0.014 g, 0.03 mmol) in DMF (0.2 ml), Lithium bromide (0.024 g, 0.3 mmol) was added at room temperature. The reaction mixture was heated and stirred at 130° C. for 1 hour. After completion of reaction (confirmed by TLC), the reaction mixture was loaded on RP Gold column and purified using acetonitrile and 0.1% Formic acid in water to give pure title compound (0.003 g, 22%).

Isomer-1_(D1E1) LCMS: m/ [symbol] 481.7 [M⁺+1].

Isomer-2_(D1E2) LCMS: m/ [symbol] 481.3 [M⁺+1].

Isomer-3_(D2E1) LCMS: m/ [symbol] 481.3 [M⁺+1].

Isomer-4_(D2E2) LCMS: m/ [symbol] 481.3 [M⁺+1].

Isomer-1_D1E1: ¹H NMR (400 MHz, MeOD): δ 1.26 (bs, 3H), 2.69 (s, 3H), 3.79 (s, 3H), 4.24 (bs, 1H), 5.48 (bs, 1H), 7.10-7.26 (m, 3H), 7.64 (bs, 1H), 8.68-8.90 (m, 3H), 9.17 (s, 1H).

Isomer-2_D1E2: ¹H NMR (400 MHz, MeOD): δ 1.28 (d, J=8.8 Hz, 3H), 2.69 (s, 3H), 3.79 (s, 3H), 4.26 (bs, 1H), 5.48 (bs, 1H), 7.11-7.26 (m, 3H), 7.64 (bs, 1H), 8.80-8.91 (m, 3H), 9.21 (bs, 1H).

Isomer-3_D2E1: ¹H NMR (400 MHz, MeOD): δ 1.41 (s, 3H), 2.53 (s, 3H), 3.66 (s, 3H), 4.26 (bs, 1H), 5.32 (d, J=11.2 Hz, 1H), 7.34 (t, J=7.6 Hz, 1H), 7.47-7.51 (m, 2H), 7.94 (d, J=7.2 Hz, 1H), 8.59 (s, 2H), 8.80 (s, 1H), 9.26 (s, 1H).

Isomer-4_D2E2: ¹H NMR (400 MHz, MeOD): δ 1.35 (s, 3H), 2.50 (s, 3H), 3.67 (s, 3H), 4.20 (bs, 1H), 5.37 (d, J=11.2 Hz, 1H), 7.32 (t, J=7.6 Hz, 1H), 7.45-7.49 (m, 2H), 7.94 (d, J=7.2 Hz, 1H), 7.64-7.68 (m, 3H), 9.22 (s, 1H).

HPLC: FR-1 (Isomer-1; D1E1): R_{T}=4.52 (98%); FR-2 (Isomer-2; D1E2): R_{T}=4.52 (100%); FR-3 (Isomer-3; D2E1): R_{T}=4.59 (100%); FR-4 (Isomer-4; D2E2): R_{T}=4.59 (99%).

### Example 148 Synthesis of 2-(2-(2-Cyanophenyl)-1,1-difluoro-2-(1-methyl-1H-pyrazol-4-yl)ethyl)-5-hydroxy-N-(isoxazol-4-yl)-1-methyl-6-oxo-1,6-dihydropyrimidine-4-carboxamide

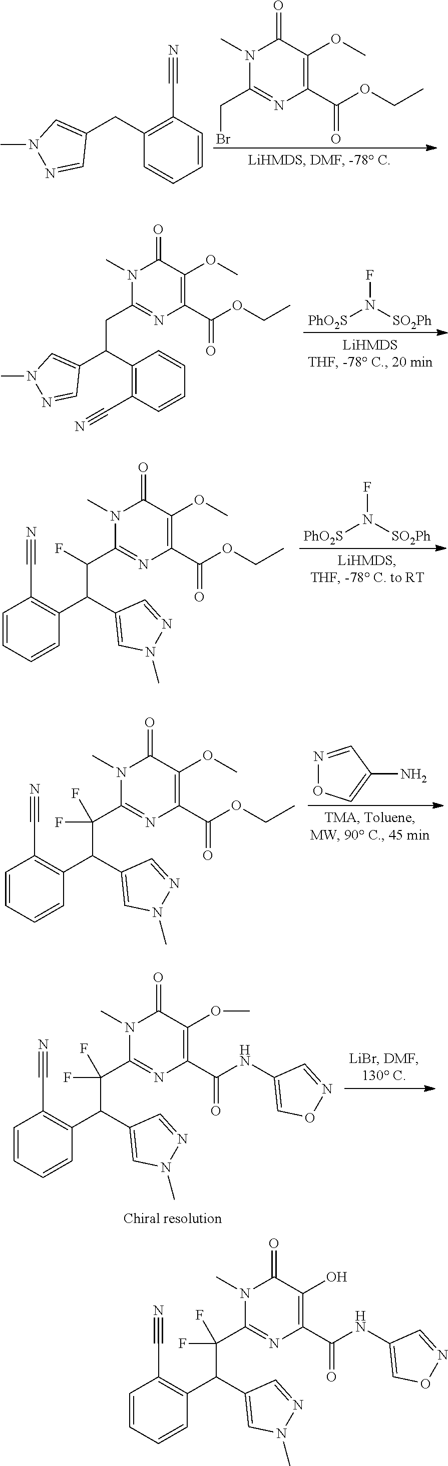

### Step 1: Ethyl 2-(2-(2-cyanophenyl)-2-(1-methyl-1H-pyrazol-4-yl)ethyl)-5-methoxy-1-methyl-6-oxo-1,6-dihydropyrimidine-4-carboxylate

A solution of 2-((1-methyl-1H-pyrazol-4-yl)methyl)benzonitrile (10 g, 50.7 mmol) and DMF:THF (100 ml, 1:1) was cooled at −78° C. To the resulting solution, LiHMDS (76.10 ml, 1M in THF, 76.1 mmol) was added over a period of 15 minutes. The reaction mixture was stirred at −78° C. for 1 hour. To it, Ethyl 2-(bromomethyl)-5-methoxy-1-methyl-6-oxo-1,6-dihydropyrimidine-4-carboxylate (15.40 g, 50.7 mmol) in DMF (50 ml) was added dropwise at −78° C. for 15 minutes. The reaction was stirred for 30 minutes. After completion of reaction (monitored by TLC), the reaction mixture was quenched with water (200 ml) and extracted with EtOAc (3×250 ml). The combined organic layer was washed with brine (150 ml), dried over anhydrous sodium sulphate, filtered, and concentrated under reduced pressure. The crude compound was purified by using Combi-flash chromatography to obtain pure title compound (2.1 g, 10%).

LCMS: m/ [symbol] 422.2 [M⁺+1].

¹H NMR (400 MHz, DMSO-d₆): δ 1.28 (t, J=6.4 Hz, 3H), 3.49-3.51 (m, 1H), 3.52 (s, 3H), 3.70-3.73 (m, 1H), 3.74 (s, 3H), 3.75 (s, 3H), 4.24 (q, J=6.0 Hz, 2H), 4.93 (t, J=3.2 Hz, 1H), 7.32 (s, 1H), 7.34 (s, 1H), 7.53 (s, 1H), 7.60 (t, J=7.2 Hz, 1H), 8.86 (t, J=8.0 Hz, 2H).

### Step 2: Ethyl 2-(2-(2-cyanophenyl)-1-fluoro-2-(1-methyl-1H-pyrazol-4-yl)ethyl)-5-methoxy-1-methyl-6-oxo-1,6-dihydropyrimidine-4-carboxylate

A solution of Ethyl 2-(2-(2-cyanophenyl)-2-(1-methyl-1H-pyrazol-4-yl)ethyl)-5-methoxy-1-methyl-6-oxo-1,6-dihydropyrimidine-4-carboxylate (1.1 g, 2.6 mmol) and THF (20 ml) was cooled at −78° C. under Nitrogen gas atmosphere. To the resulting solution, LiHMDS (3.91 ml, 1M in THF, 3.9 mmol) was added dropwise over a period of 15 minutes. The reaction mixture was stirred at −78° C. for 1 hour. A solution of N-fluoro-N-(phenylsulfonyl)benzenesulfonamide (0.823 g, 2.6 mmol) in THF (10 ml) was added dropwise at −78° C. for 15 minutes. The reaction was stirred for 30 minutes. After completion of reaction (monitored by TLC), the reaction mixture was quenched with water (20 ml) and extracted with EtOAc (3×50 ml). The combined organic layer was washed with brine (30 ml), dried over anhydrous sodium sulphate, filtered, and concentrated under reduced pressure. The crude compound was purified by using Combi-flash chromatography to obtain pure title compound (0.7 g, 61%).

LCMS: m/ [symbol] 440.20 [M⁺+1].

### Step 3: Ethyl 2-(2-(2-cyanophenyl)-1,1-difluoro-2-(1-methyl-1H-pyrazol-4-yl)ethyl)-5-methoxy-1-methyl-6-oxo-1,6-dihydropyrimidine-4-carboxylate

The LiHMDS (2.39 ml, 1M in THF, 2.4 mmol) was drop wise added to a stirred solution of Ethyl 2-(2-(2-cyanophenyl)-1-fluoro-2-(1-methyl-1H-pyrazol-4-yl)ethyl)-5-methoxy-1-methyl-6-oxo-1,6-dihydropyrimidine-4-carboxylate (0.7 g, 1.6 mmol) in THF (20 ml) at −78° C. for 15 minutes. Reaction mixture was stirred at −78° C. for 1 hour. A solution of N-fluoro-N-(phenylsulfonyl)benzenesulfonamide (0.5 g, 1.6 mmol) in THF (10 ml) was added dropwise at −78° C. for 15 minutes. The reaction was stirred for 30 minutes. After completion of reaction (monitored by TLC), the reaction mixture was quenched with water (20 ml) and extracted with EtOAc (3×50 ml). The combined organic layer was washed with brine (30 ml), dried over anhydrous sodium sulphate, filtered, and concentrated under reduced pressure. The crude compound was purified by using Combi-flash chromatography to obtain pure title compound (0.6 g, 82%).

LCMS: m/ [symbol] 458 [M⁺+1].

¹H NMR (400 MHz, DMSO-d₆): δ 1.29 (t, J=6.4 Hz, 3H), 3.59 (s, 3H), 3.78 (s, 3H), 3.86 (s, 3H), 4.28 (q, J=6.8 Hz, 2H), 5.62 (t, J=3.2 Hz, 1H), 7.45-7.49 (m, 2H), 7.68 (t, J=7.6 Hz, 1H), 7.77-7.84 (m, 3H).

### Step 4:2-(2-(2-Cyanophenyl)-1,1-difluoro-2-(1-methyl-1H-pyrazol-4-yl)ethyl)-N-(isoxazol-4-yl)-5-methoxy-1-methyl-6-oxo-1,6-dihydropyrimidine-4-carboxamide

A solution of Ethyl 2-(2-(2-cyanophenyl)-1,1-difluoro-2-(1-methyl-1H-pyrazol-4-yl)ethyl)-5-methoxy-1-methyl-6-oxo-1,6-dihydropyrimidine-4-carboxylate (0.6 g, 1.31 mmol), Isooxazole amine (0.165 g, 2.0 mmol) and Toluene (10 ml) was cooled at 0° C. To the resulting solution, Trimethyl aluminum (1.31 ml, 2M in Toluene, 2.6 mmol) was added slowly. The reaction mixture was heated at 80° C. and stirred for 1 hour under Microwave irradiation. After completion of reaction (confirmed by TLC), the reaction mixture was loaded on RP Gold column and purified using acetonitrile and 0.1% formic acid in water to give pure title compound (0.170 g, 26%).

LCMS: m/ [symbol] 496.0 [M⁺+1].

Chiral HPLC Method: The diastereomers of title compound was resolved by Chiral SFC [FR1 and FR:2 (CHIRALCEL OX-H (250*21 mm; 5 u; LIQUID.CO2+0.1% DEA in methanol (75:25) to furnish the enantiomer pure compounds.

Isomer-1_LCMS: m/ [symbol] 496.2 [M⁺+1].

Isomer-2_LCMS: m/ [symbol] 496.3 [M⁺+1].

Chiral HPLC: FR-1 (Isomer-1): RT=5.28; FR-2 (Isomer-2): RT=5.70.

### Step 5: 2-(2-(2-Cyanophenyl)-1,1-difluoro-2-(1-methyl-1H-pyrazol-4-yl)ethyl)-5-hydroxy-N-(isoxazol-4-yl)-1-methyl-6-oxo-1,6-dihydropyrimidine-4-carboxamide

The Lithium bromide (0.104 g, 1.2 mmol) was added to a solution of 2-(2-(2-Cyanophenyl)-1,1-difluoro-2-(1-methyl-1H-pyrazol-4-yl)ethyl)-N-(isoxazol-4-yl)-5-methoxy-1-methyl-6-oxo-1,6-dihydropyrimidine-4-carboxamide (0.060 g, 1.2 mmol) and DMF (1.2 ml). The reaction mixture was heated at 130° C. for 1 hour under Microwave irradiation. After completion of reaction (confirmed by TLC), the reaction mixture was loaded on RP Gold column and purified using acetonitrile and 0.1% formic acid in water to give pure title compound (0.039 g, 66%).

Isomer-1_LCMS: m/ [symbol] 482.51 [M⁺+1].

Isomer-2_LCMS: m/ [symbol] 482.51 [M⁺+1].

Isomer-1: ¹H NMR (400 MHz, DMSO-d₆): δ 3.63 (s, 3H), 3.75 (s, 3H), 5.87-5.96 (m, 1H), 7.51 (t, J=2.4 Hz, 1H), 7.54 (s, 1H), 7.69 (s, 1H), 7.84 (s, 1H), 7.86 (s, 1H), 8.86 (s, 1H), 9.30 (s, 1H), 10.28 (s, 1H).

Isomer-2: ¹H NMR (400 MHz, DMSO-d₆): δ 3.63 (s, 3H), 3.75 (S, 3H), 5.87-5.96 (m, 1H), 7.51 (t, J=2.4 Hz, 1H), 7.54 (s, 1H), 7.69 (s, 1H), 7.84 (s, 1H), 7.86 (s, 1H), 8.86 (s, 1H), 9.30 (s, 1H), 10.28 (s, 1H).

HPLC: FR-1 (Isomer-1): R_{T}=4.59 (100%); FR-2 (Isomer-2): R_{T}=4.59 (100%).

### Example 149 Synthesis of 2-(1-(2-Cyanophenyl)-1-(1-methyl-1H-pyrazol-4-yl)propan-2-yl)-5-hydroxy-1-ethyl-N-(isoxazol-4-yl)-6-oxo-1,6-dihydropyrimidine-4-carboxamide

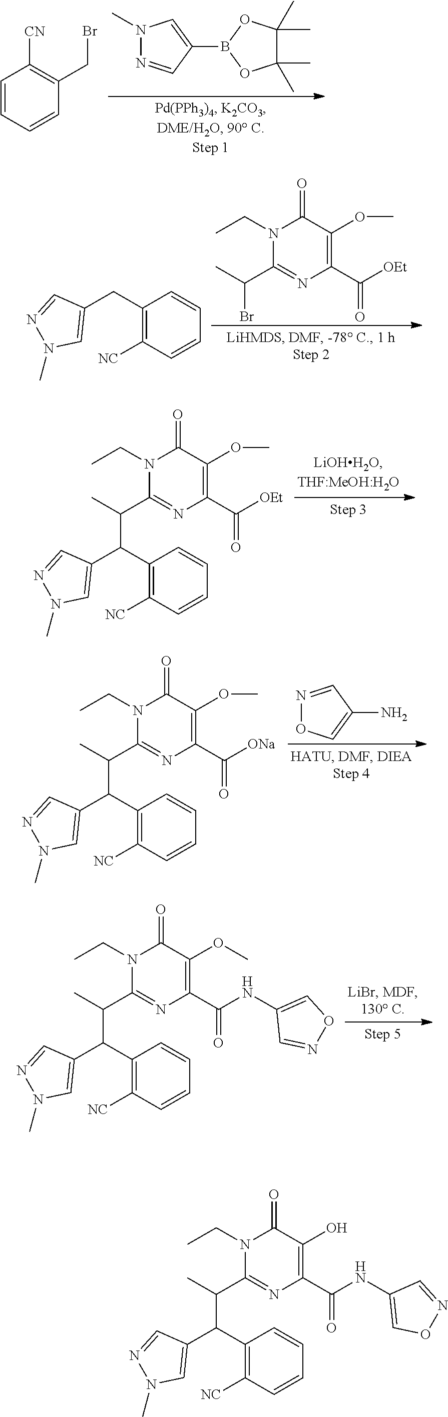

### Step 1: 2-((1-Methyl-1H-pyrazol-4-yl)methyl) benzonitrile

A mixture of 1-(Bromomethyl)-2-chlorobenzene (10.0 g, 51.0 mmol), 1-Methyl-4-(4,4,5,5-tetramethyl-1,3,2-dioxaborolan-2-yl)-1H-pyrazole (10.65 g, 51.0 mmol) and Potassium carbonate (14.09 g, 102.4 mmol) in a mixture of 1,2-Dimethoxyethane:water (180 ml, 7:3) was purged for 20 minutes with Argon gas. To it, Tetrakis (2.94 g, 2.6 mmol) was added and purging was continued for another 10 minutes. The reaction mixture was heated in a sealed tube at 90° C. for 2 hours. After completion of reaction (monitored by TLC), the reaction mixture was filtered through Celite bed and filtrate was washed with EtOAc (3×250 ml). The combined organic layer was washed with brine (300 ml), dried over anhydrous sodium sulphate, filtered, and concentrated under reduced pressure. The crude compound was purified by using Combi-flash chromatography to obtain pure title compound (6 g, 60%).

LCMS: m/ [symbol] 197.91[M⁺+1].

### Step 2: Ethyl 2-(1-(2-cyanophenyl)-1-(1-methyl-H-pyrazol-4-yl)propan-2-yl)-1-ethyl-5-methoxy-6-oxo-1,6-dihydropyrimidine-4-carboxylate

A solution of LiHMDS (7.5 ml, 1M in THF, 7.5 mmol) was cooled to −78° C. under Nitrogen atmosphere. To it, a solution of 2-((1-methyl-1H-pyrazol-4-yl)methyl) benzonitrile (0.88 g, 4.5 mmol) in DMF (4 ml) was added at −78° C. for a period of 15 minutes. The reaction mixture was stirred at −78° C. for another 10 minutes. Ethyl 2-(1-bromoethyl)-1-ethyl-5-methoxy-6-oxo-1,6-dihydropyrimidine-4-carboxylate (1 g, 3.0 mmol) in DMF (6 ml) was added dropwise at −78° C. for 15 minutes. After completion of the reaction (30 minutes), reaction mixture was quenched with saturated solution of aq. NH₄Cl (10 ml) and extracted with EtOAc (3×30 ml). The combined organic layer was washed with brine (30 ml), dried over anhydrous sodium sulphate, filtered, and concentrated under reduced pressure. The crude compound was purified by using Combi-flash chromatography which gives partially pure product which was used in the next step without further purification.

Isomer-1 (D1)_LCMS: m/ [symbol] : 450.4[M⁺+1].

Isomer-2 (D2)_LCMS m/ [symbol] : 450.3 [M⁺+1].

### Step 3: sodium 2-(1-(2-Cyanophenyl)-1-(1-methyl-1H-pyrazol-4-yl)propan-2-yl)-1-ethyl-5-methoxy-6-oxo-1,6-dihydropyrimidine-4-carboxylate

The sodium hydroxide (0.46 g, 1.166 mmol) was added to a stirred solution of Ethyl 2-(1-(2-cyanophenyl)-1-(1-methyl-1H-pyrazol-4-yl)propan-2-yl)-1-ethyl-5-methoxy-6-oxo-1,6-dihydropyrimidine-4-carboxylate (0.350 g, 0.777 mmol) and methanol:THF:water (8 ml, 1:1:1) at room temperature. The reaction mixture was stirred at room temperature for 1 hour. After completion of reaction (confirmed by TLC), the reaction mixture was concentrated under reduced pressure to obtain crude title compound (0.370 g) which was used for next step without further purification.

Isomer-1 (D1)_LCMS: m/ [symbol] : 422.24 [M⁺+1].

Isomer-2 (D2)_LCMS: m/ [symbol] : 422.30[M⁺+1].

### Step 4: 2-(1-(2-Cyanophenyl)-1-(1-methyl-1H-pyrazol-4-yl)propan-2-yl)-1-ethyl-N-(isoxazol-4-yl)-5-methoxy-6-oxo-1,6-dihydropyrimidine-4-carboxamide

To a stirred solution of sodium 2-(1-(2-Cyanophenyl)-1-(1-methyl-1H-pyrazol-4-yl)propan-2-yl)-1-ethyl-5-methoxy-6-oxo-1,6-dihydropyrimidine-4-carboxylate (0.35 g, 0.8 mmol) in DMF (3.5 ml) was added HATU (0.450 g, 1.2 mmol), Isoxazol-4-amine (0.079 g, 1.0 mmol) at room temperature. The reaction mixture was stirred at room temperature for 30 minutes. Then, DIPEA (0.35 ml, 2.0 mmol) was added and reaction mixture was allowed to stir for another 1 hour. After completion of reaction (monitor by TLC), the reaction mixture was diluted with water (10 ml) and aqueous layer was extracted with EtOAc (3×10 ml). The combined organic layer was washed with brine (10 ml), dried over anhydrous sodium sulphate, filtered, and concentrated under reduced pressure. The crude compound was purified by Combi-flash column chromatography to give pure title compound (0.15 g).

The Diastereomer mixture (0.15 g) was separated by using Reverse Phase-HPLC to get two separated Diastereomers as D1 (0.09 g) and D2 (0.05 g).

Isomer-1 (D1)_LCMS: m/ [symbol] : 488.6 [M⁺+1].

Isomer-2 (D2)_LCMS: m/ [symbol] : 488.7 [M⁺+1].

Chiral HPLC Method: The diastereomers of title compound was resolved by Chiral HPLC [D1: (CHIRALPAK IB-N(250*21) mm, 5 u; 0.1% DEA in n-Hexane+0.1% DEA in IPA:ACN (70:30)] [D2: (Chiralpak IC (250*21.0) mm, 5 u; Liquid Carbon dioxide (Liq. CO2)+0.1% DEA in Propane-2-ol: acetonitrile (50:50)] to furnish the enantiopure compounds.

Chiral HPLC: FR-1 (Isomer-1; D1E1): R_{T}=7.50 (99%); FR-2 (Isomer-2; D1E2): R_{T}=8.13 (100%); FR-3 (Isomer-3; D2E1): R_{T}=4.64(100%); FR-4 (Isomer-4; D2E2): RT=6.07 (100%).

### STEP 5: 2-(1-(2-Cyanophenyl)-1-(1-methyl-1H-pyrazol-4-yl)propan-2-yl)-5-hydroxy-1-ethyl-N-(isoxazol-4-yl)-6-oxo-1,6-dihydropyrimidine-4-carboxamide

The Lithium bromide (0.106 g, 1.23 mmol) was added to a stirred solution of 2-(1-(2-cyanophenyl)-1-(1-methyl-1H-pyrazol-4-yl)propan-2-yl)-1-ethyl-N-(isoxazol-4-yl)-5-methoxy-6-oxo-1,6-dihydropyrimidine-4-carboxamide (0.040 g, 0.1 mmol) and DMF (0.5 ml) under nitrogen atmosphere. The reaction mixture was heated at 130° C. for 1 hour under Microwave irradiation. After completion of reaction (confirmed by TLC), the reaction mixture was loaded on RP Gold column and purified using acetonitrile and 0.1% formic acid in water to give pure title compound (0.020 g, 51%).

Isomer-1_(D1E1)_LCMS: m/ [symbol] 474.3 [M⁺+1].

Isomer-2_(D1E2) LCMS: m/ [symbol] 474.3[M⁺+1].

Isomer-3_(D2E1)_LCMS: m/ [symbol] 474.3[M⁺+1].

Isomer-4_(D2E2)_LCMS: m/ [symbol] 474.3 [M⁺+1].

Isomer-1_D1E1: ¹H NMR (400 MHz, DMSO-d₆): δ 1.23 (s, 3H), 1.35 (d, J=6.0 Hz, 3H), 3.81 (s, 3H), 4.06-4.09 (m, 2H), 4.17-4.19 (m, 1H), 5.04 (d, J=6.4 Hz, 1H), 7.24 (t, J=7.6 Hz, 1H), 7.55-7.59 (m, 2H), 7.63 (s, 1H), 7.82 (s, 2H), 7.85 (s, 1H), 8.87 (s, 1H), 9.31 (s, 1H), 10.49 (s, 1H), 11.23 (s, 1H).

Isomer-2_D1E2: ¹H NMR (400 MHz, DMSO-d₆): δ 1.23 (s, 3H), 1.35 (d, J=6.0 Hz, 3H), 3.81 (s, 3H), 4.00-4.09 (m, 2H), 4.18 (m, 1H), 5.05 (d, J=7.2 Hz, 1H), 7.22 (t, J=7.6 Hz, 1H), 7.57 (d, J=7.6 Hz, 2H), 7.63 (s, 1H), 7.82 (s, 2H), 7.85 (s, 1H), 8.87 (s, 1H), 9.30 (s, 1H), 10.47 (s, 1H), 11.23 (s, 1H).

Isomer-3_D2E1: ¹H NMR (400 MHz, DMSO-d₆): δ 1.23 (s, 3H), 1.36 (d, J=6.0 Hz, 3H), 3.83 (s, 3H), 4.05-4.09 (m, 2H), 4.15-4.19 (m, 1H), 5.04 (d, J=6.4 Hz, 1H), 7.22 (t, J=7.6 Hz, 1H), 7.60 (d, J=7.6 Hz, 2H), 7.65 (s, 1H), 7.83 (s, 2H), 7.86 (s, 1H), 8.88 (s, 1H), 9.32 (s, 1H), 10.49 (s, 1H), 11.24 (s, 1H).

Isomer-4_D2E2: ¹H NMR (400 MHz, DMSO-d₆): δ 1.25 (s, 3H), 1.36 (d, J=6.0 Hz, 3H), 3.83 (s, 3H), 4.09-4.11 (m, 2H), 4.1-4.22 (m, 1H), 5.05 (d, J=6.4 Hz, 1H), 7.22 (t, J=7.6 Hz, 1H), 7.57 (d, J=7.6 Hz, 2H), 7.65 (s, 1H), 7.83 (s, 2H), 7.86 (s, 1H), 8.88 (s, 1H), 9.32 (s, 1H), 10.49 (s, 1H), 11.24 (s, 1H).

HPLC: FR-1 (Isomer-1; D1E1): R_{T}=4.53 (100%); FR-2 (Isomer-2; D1E2): R_{T}=4.53 (95%); FR-3 (Isomer-3; D2E1): R_{T}=4.50 (99%); FR-4 (Isomer-4; D2E2): R_{T}=4.53 (100%).

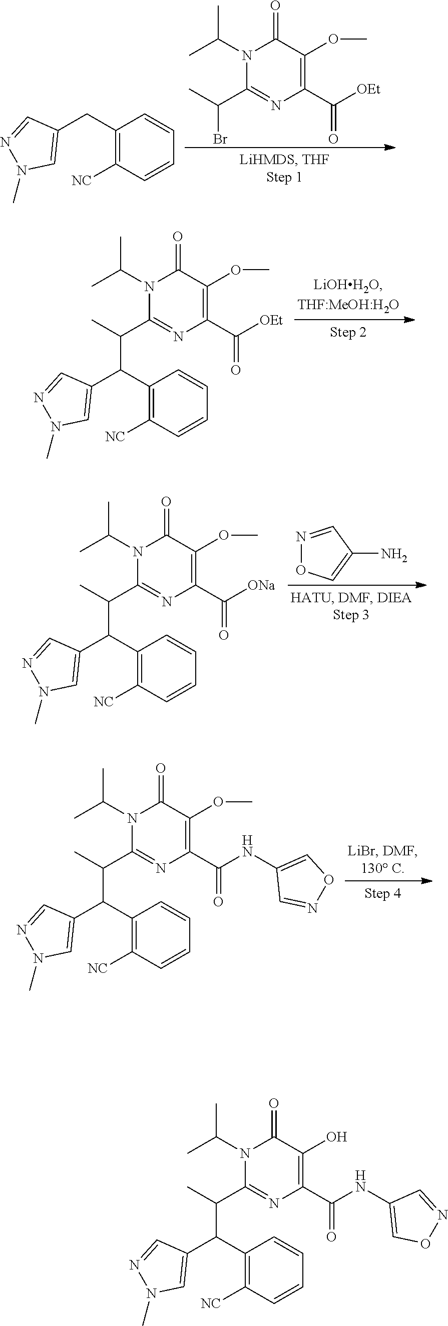

### Step 1: Ethyl 2-(1-(2-cyanophenyl)-1-(1-methyl-1H-pyrazol-4-yl)propan-2-yl)-1-isopropyl-5-methoxy-6-oxo-1,6-dihydropyrimidine-4-carboxylate

To Solution of 1M LiHMDS in THF (14.19 ml, 14.2 mmol) was cooled to −78° C. under Nitrogen gas atmosphere. To it, a solution of 2-((1-methyl-1H-pyrazol-4-yl)methyl)benzonitrile (1.0 g, 5.1 mmol) in DMF (4 ml) was added drop wise over a period of 15 minutes. The reaction mixture was stirred at −78° C. for another 10 minutes and solution of Ethyl 2-(1-bromoethyl)-1-isopropyl-5-methoxy-6-oxo-1,6-dihydropyrimidine-4-carboxylate (1.17 g, 3.4 mmol) in DMF (6 ml) was added dropwise over 15 minutes. After completion of the reaction (30 minutes), the reaction mixture was quenched with saturated solution of aq. NH₄Cl (20 ml) and extracted with EtOAc (3×30 ml). The combined organic layer was washed with brine (30 ml), dried over anhydrous sodium sulphate, filtered, and concentrated under reduced pressure. The crude compound was purified by using Combi-flash to give partially pure title product, which was used in the next step without further purification.

Isomer-1 (D1)_LCMS: m/ [symbol] : 464.0 [M⁺+1].

Isomer-2 (D2)_LCMS: m/ [symbol] : 464.0 [M⁺+1].

### Step 2: 2-(1-(2-Cyanophenyl)-1-(1-methyl-1H-pyrazol-4-yl)propan-2-yl)-1-isopropyl-5-methoxy-6-oxo-1,6-dihydropyrimidine-4-carboxylic acid

sodium hydroxide (0.090 g, 2.26 mmol) was added to a stirred solution of Ethyl 2-(1-(2-cyanophenyl)-1-(1-methyl-1H-pyrazol-4-yl)propan-2-yl)-1-isopropyl-5-methoxy-6-oxo-1,6-dihydropyrimidine-4-carboxylate (0.700 g, 1.5 mmol) and methanol:THF:water (1:1:1, 10.5 ml) at room temperature. The reaction mixture was stirred at room temperature for 1 hour. After completion of reaction (confirmed by TLC), the reaction mixture was concentrated under reduced pressure to obtain crude title product (0.6 g, 84%) which was used in the next step without further purification.

Isomer-1 (D1)_LCMS: m/ [symbol] : 436.4 [M⁺+1].

Isomer-2 (D2)_LCMS: m/ [symbol] : 436.4 [M⁺+1].

### Step 3: 2-(1-(2-Cyanophenyl)-1-(1-methyl-1H-pyrazol-4-yl)propan-2-yl)-1-isopropyl-N-(isoxazol-4-yl)-5-methoxy-6-oxo-1,6-dihydropyrimidine-4-carboxamide

To a stirred solution of sodium 2-(1-(2-Cyanophenyl)-1-(1-methyl-1H-pyrazol-4-yl)propan-2-yl)-1-isopropyl-5-methoxy-6-oxo-1,6-dihydropyrimidine-4-carboxylate (0.420 g, 0.9 mmol) in DMF (4.2 ml) was added HATU (0.419 g, 1.1 mmol), Isoxazol-4-amine (0.143 g, 1.2 mmol) at room temperature. The reaction mixture was stirred at room temperature for 30 minutes. Then, DIPEA (0.31 ml, 1.8 mmol) was added and reaction mixture was allowed to stir for another 1 hour. After completion of reaction (monitor by TLC), the reaction mixture was diluted with water (10 ml) and aqueous layer was extracted with EtOAc (3×10 ml). The combined organic layer was washed with brine (10 ml), dried over anhydrous sodium sulphate, filtered, and concentrated under reduced pressure. The crude compound was purified by Combi-flash column chromatography to give pure title compound (0.15 g, 32%).

The Diastereomer mixture (0.15 g) was separated by using Reverse Phase-HPLC to get two separated Diastereomers as D1 (0.10 g) and D2 (0.05 g).

Isomer-1 (D1)_LCMS: m/ [symbol] : 502.0 [M⁺+1].

Isomer-2 (D2)_LCMS: m/ [symbol] : 502.0[M⁺+1].

Chiral HPLC Method: The diastereomers of title compound was resolved by Chiral HPLC [D1: (CHIRALPAK IB-N(250*21) mm, 5 u; Propane-2-ol:acetonitrile (70:30) in Hexanes+0.1% DEA)] [D2: (CHIRALPAK IB-N(250*21) mm, 5 u; Propane-2-ol in hexanes+0.1% DEA)] to furnish the enantiopure compounds.

Chiral HPLC: FR-1 (Isomer-1; D1E1): RT=6.45(99%); FR-2 (Isomer-2; D1E2): R_{T}=8.13(100%); FR-3 (Isomer-3; D2E1): R_{T}=9.03(96%); FR-4 (Isomer-4; D2E2): RT=9.91(95%).

### Step 4: 2-(1-(2-Cyanophenyl)-1-(1-methyl-1H-pyrazol-4-yl)propan-2-yl)-5-hydroxy-1-isopropyl-N-(isoxazol-4-yl)-6-oxo-1,6-dihydropyrimidine-4-carboxamide

The Lithium bromide (0.130 g, 1.49 mmol) was added to a solution of 2-(1-(2-Cyanophenyl)-1-(1-methyl-1H-pyrazol-4-yl)propan-2-yl)-1-isopropyl-N-(isoxazol-4-yl)-5-methoxy-6-oxo-1,6-dihydropyrimidine-4-carboxamide (0.050 g, 0.1 mmol) and DMF (0.5 ml) under nitrogen atmosphere. The reaction mixture was heated at 130° C. for 1 hour under Microwave irradiation. After completion of reaction (confirmed by TLC), the reaction mixture was loaded on RP Gold column and purified using acetonitrile and 0.1% formic acid in water to give pure title compound (0.027 g, 100%).

Isomer-1_(D1E1)_LCMS: m/ [symbol] 488.3 [M⁺+1].

Isomer-2_(D1E2)_LCMS: m/ [symbol] 488.3 [M⁺+1].

Isomer-3_(D2E1)_LCMS: m/ [symbol] 488.3 [M⁺+1].

Isomer-4_(D2E2)_LCMS: m/ [symbol] 488.3 [M⁺+1].

Isomer-1_D1E1: ¹H NMR (400 MHz, DMSO-d₆): δ 1.34 (d, J=5.6 Hz, 3H), 1.50 (d, J=5.6 Hz, 3H), 1.56 (d, J=6.0 Hz, 3H), 3.83 (s, 3H), 4.24-4.28 (m, 1H), 4.94-4.97 (m, 1H), 5.10 (d, J=10.8 Hz, 1H), 7.23 (t, J=7.2 Hz, 1H), 7.57-7.63 (m, 3H), 7.76 (d, J=7.6 Hz, 1H), 7.82 (s, 1H), 8.88 (s, 1H), 9.30 (s, 1H), 10.40 (s, 1H), 11.02 (s, 1H).

Isomer-2_D1E2: ¹H NMR (400 MHz, DMSO-d₆): δ 1.34 (d, J=6.4 Hz, 3H), 1.49 (d, J=6.4 Hz, 3H), 1.54 (d, J=6.4 Hz, 3H), 3.81 (s, 3H), 4.22-4.28 (m, 1H), 4.92-4.95 (m, 1H), 5.10 (d, J=10.8 Hz, 1H), 7.20 (t, J=7.2 Hz, 1H), 7.55-7.61 (m, 3H), 7.75 (d, J=7.6 Hz, 1H), 7.81 (s, 1H), 8.86 (s, 1H), 9.29 (s, 1H), 10.42 (s, 1H), 11.02 (s, 1H).

Isomer-3_D2E1: ¹H NMR (400 MHz, DMSO-d₆): δ 1.15 (d, J=5.2 Hz, 3H), 1.30 (d, J=6.0 Hz, 3H), 1.51 (d, J=4.8 Hz, 3H), 3.66 (s, 3H), 4.10-4.20 (m, 1H), 4.80-4.90 (m, 1H), 5.01 (d, J=10.0 Hz, 1H), 7.13 (s, 1H), 7.39 (s, 1H), 7.49 (t, J=7.2 Hz, 1H), 7.81 (t, J=7.2 Hz, 1H), 7.86 (d, J=7.2 Hz, 1H), 8.02-8.03 (m, 1H), 8.87 (s, 1H), 9.35 (s, 1H), 10.33 (s, 1H), 10.96 (s, 1H).

Isomer-4_D2E2: ¹H NMR (400 MHz, DMSO-d₆): δ 1.15 (d, J=6.0 Hz, 3H), 1.29 (d, J=4.4 Hz, 3H), 1.51 (d, J=5.6 Hz, 3H), 3.66 (s, 3H), 4.10-4.20 (m, 1H), 4.80-4.90 (m, 1H), 5.01 (d, J=10.4 Hz, 1H), 7.13 (s, 1H), 7.39 (s, 1H), 7.49 (t, J=7.6 Hz, 1H), 7.81 (t, J=7.6 Hz, 1H), 7.86 (d, J=7.6 Hz, 1H), 8.02-8.03 (m, 1H), 8.86 (s, 1H), 9.35 (s, 1H), 10.32 (s, 1H), 10.96 (s, 1H).

HPLC: FR-1 (Isomer-1; D1E1): R_{T}=4.70 (97%); FR-2 (Isomer-2; D1E2): R_{T}=4.79 (98%); FR-3 (Isomer-3; D2E1): R_{T}=4.89 (99%); FR-4 (Isomer-4; D2E2): R_{T}=4.91 (100%).

### Example 151

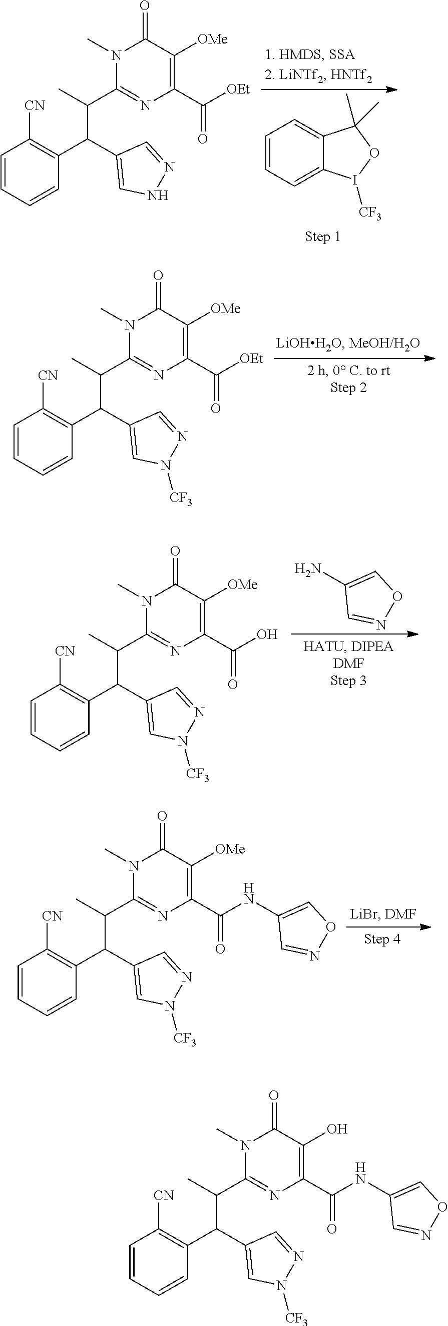

### Step 1 ethyl 2-(1-(2-cyanophenyl)-1-(1-(trifluoromethyl)-1H-pyrazol-4-yl)propan-2-yl)-5-methoxy-1-methyl-6-oxo-1,6-dihydropyrimidine-4-carboxylate

A mixture of silica sulfuric acid (SSA, 24.2 mg) and ethyl 2-(1-(2-cyanophenyl)-1-(1H-pyrazol-4-yl)propan-2-yl)-5-methoxy-1-methyl-6-oxo-1,6-dihydropyrimidine-4-carboxylate (2.00 g, 4.8 mmol) in LHMDS (48.0 mL) was stirred at reflux (125° C.) for 2 h. The mixture was cooled to room temperature, then DCM was added and the reaction mixture was filtered and the filtrate was concentrated in vacuo. The resulting material was dissolved in DCM (2.7 mL), then lithium bis((trifluoromethyl)sulfonyl)amide (24.8 mg, 0.08 mmol) was added. After the reaction mixture was shaken, 3, 3-dimethyl-1-(trifluoromethyl)-1,3-dihydro-1l3-benzo[d][1,2]iodaoxole (1.43 g, 4.3 mmol) was added followed by 1,1,1-trifluoro-N-((trifluoromethyl)sulfonyl)methanesulfonamide (146.0 mg, 0.5 mmol) were added. The resulting clear solution was then stirred at 35° C. overnight. The mixture was concentrated in vacuo and the residue was purified by silica gel column chromatography, eluted with dichloromethane/EtOAc (2:1) to afford the product as a yellow solid (650 mg, 27% yield)

ESI-MS m/z 490.2 [M+H]⁺.

### Step 2 2-(1-(2-cyanophenyl)-1-(1-(trifluoromethyl)-1H-pyrazol-4-yl)propan-2-yl)-5-methoxy-1-methyl-6-oxo-1,6-dihydropyrimidine-4-carboxylic acid

To a stirred solution of ethyl 2-(1-(2-cyanophenyl)-1-(1-(trifluoromethyl)-1H-pyrazol-4-yl)propan-2-yl)-5-methoxy-1-methyl-6-oxo-1,6-dihydropyrimidine-4-carboxylate (0.650 g, 1.3 mmol) in THF (10 mL) was added LiOH·H₂O (0.084 g, 2.0 mmol) in water (2 mL). The reaction mixture was stirred at rt for 2 h at which point the mixture was concentrated in vacuo and the resulting product was used directly without further purification

ESI-MS m/z 462.2 [M+H]⁺.

### Step 3 2-(1-(2-cyanophenyl)-1-(1-(trifluoromethyl)-1H-pyrazol-4-yl)propan-2-yl)-N-(isoxazol-4-yl)-5-methoxy-1-methyl-6-oxo-1,6-dihydropyrimidine-4-carboxamide

To a stirred solution of 2-(1-(2-cyanophenyl)-1-(1-(trifluoromethyl)-1H-pyrazol-4-yl)propan-2-yl)-5-methoxy-1-methyl-6-oxo-1,6-dihydropyrimidine-4-carboxylic acid (0.560 g, 1.2 mmol) and 1,2-oxazol-4-amine hydrochloride (0.160 g, 1.3 mmol) in DMF (6 mL) was added HATU (0.597 g, 1.6 mmol) followed by DIPEA (0.781 g, 6.1 mmol) dropwise. The resulting mixture was stirred at rt for 1 h at which point it was diluted with water (50 mL) and the product was extracted with EtOAc (3×30 mL). The organic layers were collected and combined then washed with brine, dried over Na₂SO₄, and concentrated in vacuo. The resulting crude material was purified by reverse phase chromatography (0% to 100% MeCN/H₂O) fractions containing product were combined and concentrated to afford the product as an off-white solid (0.520 g, 81% yield).

ESI-MS m/z 528.2 [M+H]⁺.

Separation of diastereomers was done at this step using reverse phase chromatography: Column: XB-Phenyl 10 um; 70% to 80% MeOH/water (0.1% NH₄HCO₃) in 40 min

Peak 1_D1 contained 340 mg of a white solid.

Peak 2_D2 Contained 114 mg of a white solid.

Enantiomers of this material were separated by Prep-chiral-HPLC:

D1: Column: CHIRALPAK IC-3, 2*25 cm, 5 m; Mobile Phase A: Hex:MTBE=1:1 (0.5% 2M NH3-MeOH), Mobile Phase B:EtOH-HPLC; Flow rate: 20 mL/min; Gradient: 5% B to 5% B in 22.5 min

Peak 1 (Isomer-1_D1E1): R_{T} 13.60 min; afforded a white solid (152 mg)

Peak 2 (Isomer-2_D1E2): R_{T} 17.82 min; afforded a white solid (150 mg)

D2: CHIRALPAK ID-3, 2*25 cm, 5 m; Mobile Phase A: Hex:MTBE=1:1 (0.5% 2M NH3-MeOH), Mobile Phase B:IPA-HPLC; Flow rate: 20 mL/min; Gradient: 25% B to 25% B in 25 min

Peak 1 (Isomer-3_D2E1): R_{T} 4.25 min; afforded a white solid (51 mg).

Peak 2 (Isomer-4_D2E2): R_{T} 13.24 min; afforded a white solid (53 mg).

Step 4 2-(1-(2-cyanophenyl)-1-(1-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide

To a solution of 1-(2-cyanophenyl)-1-(1-(trifluoromethyl)-1H-pyrazol-4-yl)propan-2-yl)-N-(isoxazol-4-yl)-5-methoxy-1-methyl-6-oxo-1,6-dihydropyrimidine-4-carboxamide (0.372 g, 4.3 mmol) dissolved in DMF (7.5 ml) was added LiBr (0.372 g, 4.3 mmol). This resulting mixture was then heated to 95° C. and stirred for 4 h at which point complete conversion to the product was observed by LCMS. The reaction was then cooled to rt and concentrated in vacuo. The resulting crude material was purified by reverse phase chromatography

Isomer-1_D1E1: Isolated a white solid (0.061 g, 41% yield).

ESI-MS m/ [symbol] : 514.2 [M+H]⁺; >98% ee

¹H NMR (400 MHz, DMSO-d₆) δ 11.19 (s, 1H), 10.45 (s, 1H), 9.30 (s, 1H), 8.87 (s, 1H), 8.67 (s, 1H), 8.13 (s, 1H), 7.84 (d, 1H), 7.65-7.59 (m, 2H), 7.27 (t, 1H), 5.11 (d, 1H), 4.24-4.20 (m, 1H), 3.59 (s, 3H), 1.34 (d, 3H).

Isomer-2_D1E2: Isolated a white solid (0.090 g, 61% yield)

ESI-MS m/ [symbol] : 514.2 [M+H]⁺; >98% ee

¹H NMR (400 MHz, DMSO-d₆) δ 11.19 (s, 1H), 10.46 (s, 1H), 9.30 (s, 1H), 8.87 (s, 1H), 8.67 (s, 1H), 8.14 (s, 1H), 7.84 (d, 1H), 7.65-7.59 (m, 2H), 7.27 (t, 1H), 5.11 (d, 1H), 4.24-4.20 (m, 1H), 3.59 (s, 3H), 1.34 (d, 3H).

Isomer-3_D2E1: Isolated a white solid (0.024 g, 47% yield)

ESI-MS m/ [symbol] : 514.2 [M+H]⁺; >98% ee

¹H NMR (400 MHz, DMSO-d₆) δ 11.23 (s, 1H), 10.32 (s, 1H), 9.32 (s, 1H), 8.87 (s, 1H), 8.34 (s, 1H), 7.95 (d, 1H), 7.88-7.79 (m, 3H), 7.51 (t, 1H), 5.02 (d, 1H), 4.08-4.03 (m, 1H), 3.54 (s, 3H), 1.16 (d, 3H).

Isomer-4_D2E2: Isolated a white solid (0.019 g, 38% yield)

ESI-MS m/ [symbol] : 514.2 [M=H]⁺; >98% ee

¹H NMR (400 MHz, DMSO-d₆) δ 11.22 (s, 1H), 10.27 (s, 1H), 9.32 (s, 1H), 8.85 (s, 1H), 8.34 (s, 1H), 7.95 (d, 1H), 7.88-7.79 (m, 3H), 7.51 (t, 1H), 5.02 (d, 1H), 4.10-4.03 (m, 1H), 3.54 (s, 3H), 1.16 (d, 3H).

### Example 152

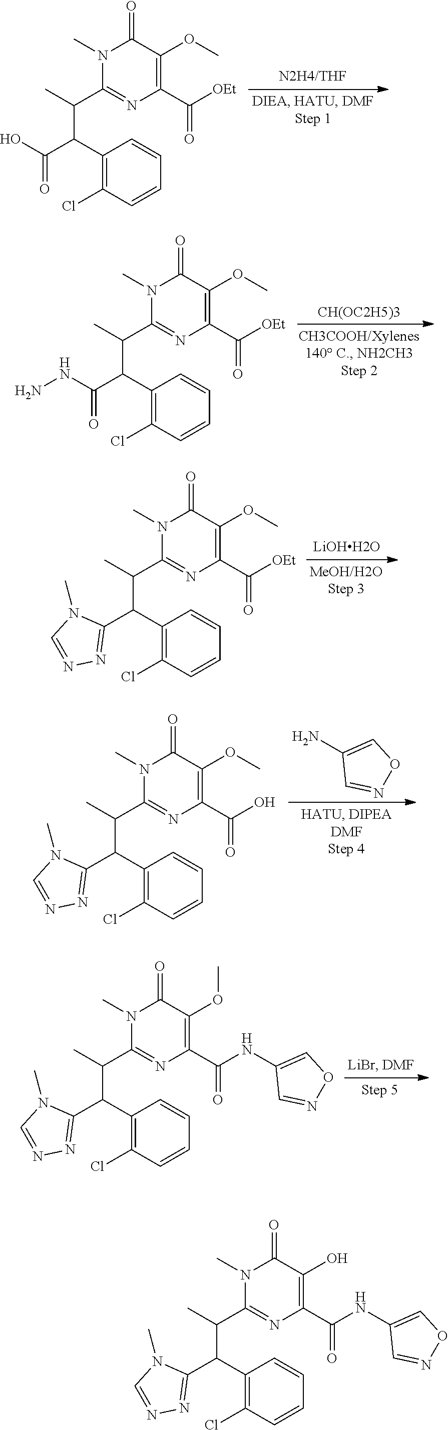

### Step 1: ethyl 2-[1-(2-chlorophenyl)-1-(hydrazinecarbonyl)propan-2-yl]-5-methoxy-1-methyl-6-oxopyrimidine-4-carboxylate

To a 0° C. stirred mixture of 2-(2-chlorophenyl)-3-[4-(ethoxycarbonyl)-5-methoxy-1-methyl-6-oxopyrimidin-2-yl]butanoic acid (1.6 g, 3.9 mmol) and HATU (2.98 g, 7.8 mmol) in DMF (16 mL) was added a solution of hydrazine (19.5 mL, 1 mol/L in THF) and DIPEA (1.52 g, 11.7 mmol) dropwise. The reaction mixture was stirred for 30 min at 0° C. then quenched by the addition of water/Ice (20 mL). The resulting mixture was extracted with EtOAc (3×60 mL) and the combined organic layers were washed with water (3×20 mL), dried over Na₂SO₄ and concentrated in vacuo. The crude product mixture was used in the next step directly without further purification.

ESI-MS m/ [symbol] : 423.0 [M+H]⁺.

### Step 2: (ethyl 2-1-(2-chlorophenyl)-1-(4-methyl-4H-1,2,4-triazol-3-yl)propan-2-yl)-5-methoxy-1-methyl-6-oxo-1,6-dihydropyrimidine-4-carboxylate)

To a stirred solution of ethyl 2-[1-(2-chlorophenyl)-1-(hydrazinecarbonyl)propan-2-yl]-5-methoxy-1-methyl-6-oxopyrimidine-4-carboxylate (2.17 g, 5.1 mmol) and triethyl orthoformate (1.52 g, 10.2 mmol) in Xylenes (18 mL) and acetic acid (3 mL) was added a solution of methylamine (5.48 mL, 1 mol/L in THF). The reaction mixture was heated to 140° C. and stirred for 2 h then cooled to rt and quenched with Ice water (20 mL). The resulting mixture was extracted with DCM/methanol (10/1, 3×50 mL) and the combined organic layer was washed with water (3×20 mL) dried over Na₂SO₄ and concentrated in Vacuo. Purification and separation of diastereomers was done at this step using reverse phase chromatography (10% to 50% MeCN/water in 10 min Flow rate.

ESI-MS m/ [symbol] : 426.0 [M+H]⁺.

Peak 1_D1 contained 174 mg of a light-yellow solid.

Peak 2_D2 Contained 422 mg of a dark-yellow solid.

### Step 3: lithium 2-(1-(2-chlorophenyl)-1-(4-methyl-4H-1,2,4-triazol-3-yl)propan-2-yl)-5-methoxy-1-methyl-6-oxo-1,6-dihydropyrimidine-4-carboxylate

To a stirred solution ethyl 2-1-(2-chlorophenyl)-1-(4-methyl-4H-1,2,4-triazol-3-yl)propan-2-yl)-5-methoxy-1-methyl-6-oxo-1,6-dihydropyrimidine-4-carboxylate (0.174 g, 0.4 mmol) in MeOH (2 mL) was added LiOH·H₂O (0.033 g, 0.8 mmol) in H₂O (0.40 mL) portion wise at 0° C. The resulting mixture was stirred at rt for 0.5 h at which point the reaction was concentered in vacuo and the resulting crude material was used in the next step with no further purification.

### Step 4: 2-(1-(2-chlorophenyl)-1-(4-methyl-4H-1,2,4-triazol-3-yl)propan-2-yl)-N-(isoxazol-4-yl)-5-methoxy-1-methyl-6-oxo-1,6-dihydropyrimidine-4-carboxamide

To a stirred solution of lithium 2-(1-(2-chlorophenyl)-1-(4-methyl-4H-1,2,4-triazol-3-yl)propan-2-yl)-5-methoxy-1-methyl-6-oxo-1,6-dihydropyrimidine-4-carboxylate (0.174 g, 0.410 mmol) and 1,2-oxazol-4-amine hydrochloride (0.069 g, 0.8 mmol) in DMF (5 mL) was added HATU (0.234 g, 0.6 mmol) followed by DIPEA (0.265 g, 2.1 mmol) dropwise. The resulting mixture was stirred at rt for 1 h at which point it was diluted with water (100 mL) and the product was extracted with EtOAc (3×50 mL). The organic layers were collected and combined then washed with brine, dried over Na₂SO₄, and concentrated in vacuo. The resulting crude material was purified by Prep-TLC (DCM/MeOH 15:1) giving the product as a dark yellow solid (0.120 g, 59% yield).

ESI-MS m/ [symbol] : 484.0 [M+H]⁺.

Enantiomers of this material were separated by Prep-chiral-HPLC:

D1: Column: ChiralPAK ID-3, 4.6*50 mm, 3 μm; Mobile Phase A: Hex:MTBE=1:1(0.1% DEA), Mobile Phase B:EtOH-HPLC; Flow rate: 1 mL/min; Gradient: 30% B

Peak 1 (Isomer-1_D1E1): R_{T} 1.44 min; afforded a light-yellow solid (82 mg)

Peak 2 (Isomer-2_D1E2): R_{T} 2.13 min; afforded a light-yellow solid (72 mg)

D2: Column: ChiralPAK ID-3, 4.6*50 mm, 3 m; Mobile Phase A: Hex:MTBE=1:1(0.1% DEA), Mobile Phase B:EtOH-HPLC; Flow rate: 1 mL/min; Gradient: 30% B

Peak 1 (Isomer-1_D1E1): R_{T} 1.24 min; afforded a light-yellow solid (102 mg)

Peak 2 (Isomer-2_D1E2): R_{T} 1.98 min; afforded a light-yellow solid (87 mg)

### Step 5: 2-[1-(2-chlorophenyl)-1-(4-methyl-1,2,4-triazol-3-yl)propan-2-yl]-5-hydroxy-1-methyl-N-(1,2-oxazol-4-yl)-6-oxopyrimidine-4-carboxamide

To a solution of 2-(1-(2-chlorophenyl)-1-(4-methyl-4H-1,2,4-triazol-3-yl)propan-2-yl)-N-(isoxazol-4-yl)-5-methoxy-1-methyl-6-oxo-1,6-dihydropyrimidine-4-carboxamide (0.087 mg, 0.2 mmol) dissolved in DMF (4.5 ml) was added LiBr (0.312 mg, 3.6 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated product as a white solid (0.012 g, 14% yield)

ESI-MS m/ [symbol] : 469.8 [M+H]⁺; >98% ee

Isomer-2_D1E2: Isolated product as a white solid (0.011 g, 16% yield)

ESI-MS m/ [symbol] : 469.8 [M+H]⁺; >95% ee

Isomer-3_D2E1: Isolated an off-white solid (0.019 g, 19% yield)

ESI-MS m/ [symbol] : 469.8 [M=H]⁺; >98% ee

Isomer-4_D2E2: Isolated an off-white solid (0.014 g, 15% Yield)

ESI-MS m/ [symbol] : 469.1 [M+H]⁺; >95% ee

Isomer-1_D1E1: ¹H NMR (400 MHz, DMSO-d₆) δ 11.20 (s, 1H), 10.62 (s, 1H), 9.34 (s, 1H), 8.95 (s, 1H), 8.43 (s, 1H), 7.67 (d, 1H), 7.24 (t, 2H), 7.14-7.10 (m, 1H), 5.35 (d, 1H), 4.31-4.26 (m, 1H), 3.73 (s, 3H), 3.49 (s, 3H), 1.39 (d, 3H).

Isomer-2_D1E2: ¹H NMR (400 MHz, DMSO-d6) δ 11.20 (s, 1H), 10.63 (s, 1H), 9.34 (s, 1H), 8.94 (s, 1H), 8.43 (s, 1H), 7.67 (d, 1H), 7.24 (t, 2H), 7.14-7.10 (m, 1H), 5.35 (d, 1H), 4.31.4.26 (m, 1H), 3.73 (s, 3H), 3.49 (s, 3H), 1.39 (d, 3H).

Isomer-3_D2E1: ¹H NMR (400 MHz, DMSO-d₆) δ 11.25 (s, 1H), 10.68 (s, 1H), 9.29 (s, 1H), 8.86 (s, 1H), 8.19 (d, 1H), 7.64 (d, 1H), 7.56-7.54 (m, 1H), 7.41-7.33 (m, 2H), 5.38 (d, 1H), 4.09-4.05 (m, 1H), 3.71 (s, 3H), 3.49 (s, 3H), 1.16 (d, 3H).

Isomer-4_D2E2: ¹H NMR (400 MHz, DMSO-d6) δ 11.25 (s, 1H), 10.62 (s, 1H), 9.30 (s, 1H), 8.86 (s, 1H), 8.20 (s, 1H), 7.63 (d, 1H), 7.55 (d, 1H), 7.41-7.33 (m, 2H), 5.37 (d, 1H), 4.09-4.05 (m, 1H), 3.71 (s, 3H), 3.48 (s, 3H), 1.16 (d, 3H).

### Example 155

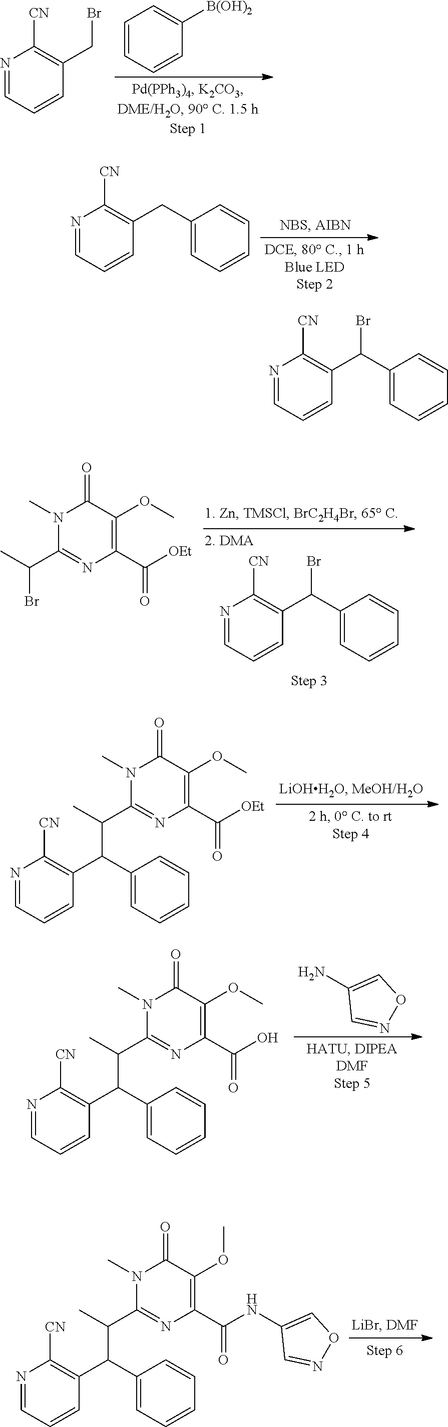

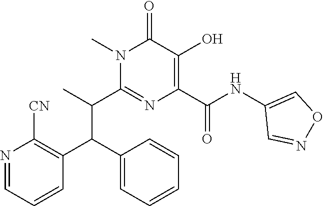 [SMILES: CC(c1nc(C(=O)Nc2cnoc2)c(O)c(=O)n1C)C(c1ccccc1)c1cccnc1C#N]

### Step 1: 3-benzyl-2-isocyanopyridine

To a stirred solution of 3-(bromomethyl)-2-isocyanopyridine (5.00 g, 25.4 mmol), potassium carbonate (7.01 g, 50.75 mmol) and phenyl boronic acid (3.71 g, 30.5 mmol) in 1,2-dimethoxy-ethan (50 mL) and water (10 mL) was added Tetrakis(triphenylphosphine)palladium (0.88 g, 0.8 mmol). The resulting mixture was heated to 90° C. and stirred for 1.5 h at which point it was allowed to cool to rt and then extracted with EtOAc (3×40 mL). The organic layers were combined and washed with brine, dried over Na₂SO₄ then concentrated in vacuo. The resulting crude material was purified by silica gel column chromatography, eluted with petroleum/EtOAc (6:1) to afford the product as a yellow solid (4.8 g, 97% yield).

ESI-MS m/z 194.9 [M+H]⁺.

### Step 2: 3-[bromo(phenyl)methyl]pyridine-2-carbonitrile

To a stirred solution of (3-benzylpyridine-2-carbonitrile) (5.25 g, 27.0 mmol) and 1-bromopyrrolidine-2,5-dione (5.29 g, 1.1 mmol) in DCM (50 mL) was added 2,2′-Azobis(2-methylpropionitrile) (1.33 g, 8.1 mmol) portion wise. The resulting mixture was heated to 80° C. and subjected to blue Light while stirring. After 1 h the mixture was allowed to cool down to room temperature and concentrated in vacuo. The residue was purified by silica gel column chromatography, eluted with pet. ether/EtOAc (1:1) to afford the product as a yellow solid (6.65 g, 90% yield)

ESI-MS m/z 272.7 [M+H]⁺.

### Step 3: 3-[2-[4-(ethoxycarbonyl)-5-methoxy-1-methyl-6-oxopyridin-2-yl]-1-phenylpropyl]-2-isocyanopyridine

Into a 100 mL 3-necked round-bottom flask were added zinc powder (1.69 g, 25.86 mmol) and DMA (10 mL) at RT. The resulting mixture was stirred for 20 minutes at 65° C. under an atmosphere of argon. To the above mixture was added dibromoethane (0.55 g, 2.91 mmol) and chlorotrimethylsilane (0.63 g, 5.8 mmol) dropwise over 5 minutes at 65° C. The resulting mixture was stirred for additional 30 minutes at 65° C. The mixture was cooled to −5° C. and a solution of 3-[bromo(phenyl)methyl]pyridine-2-carbonitrile (5.31 g, 19.4 mmol) and ethyl 6-(1-bromoethyl)-3-methoxy-1-methyl-2-oxopyridine-4-carboxylate (2.04 g, 6.4 mmol) in DMA (10 mL) was added dropwise. The resulting mixture was stirred for additional 1 h at rt and then cooled to 0° C. and quenched with saturated ammonium chloride. The product was extracted with EtOAc and the combined organic layer was washed with water, dried over Na₂SO₄ then concentrated in vacuo. After filtration, the filtrate was concentrated under reduced pressure. The resulting crude material was purified by silica gel column chromatography, eluted with EtOAc in petroleum ether (40%-60%) to afford the product as an orange oil (1.9 g, 22% yield).

ESI-MS m/z 432.8 [M+H]⁺.

### Step 4: lithio 2-[1-(2-cyanopyridin-3-yl)-1-phenylpropan-2-yl]-5-methoxy-1-methyl-6-oxopyrimidine-4-carboxylate

To a stirred solution of ethyl 2-[1-(2-cyanopyridin-3-yl)-1-phenylpropan-2-yl]-5-methoxy-1-methyl-6-oxopyrimidine-4-carboxylate (1.8 g, 4.2 mmol) in MeOH (20 mL) was added LiOH·H₂O (0.35 g, 8.3 mmol) in H₂O (4 mL). The reaction mixture was stirred at rt for 2 h at which point the mixture was concentrated in vacuo and the resulting product was used directly without further purification.

ESI-MS m/z 405.2 [M+H-Li]⁺.

### Step 5: 2-[1-(2-cyanopyridin-3-yl)-1-phenylpropan-2-yl]-5-methoxy-1-methyl-N-(1,2-oxazol-4-yl)-6-oxopyrimidine-4-carboxamide

To a stirred solution of lithio 2-[1-(2-cyanopyridin-3-yl)-1-phenylpropan-2-yl]-5-methoxy-1-methyl-6-oxopyrimidine-4-carboxylate (1.92 g, 4.7 mmol) and 1,2-oxazol-4-amine hydrochloride (0.79 g, 9.4 mmol in DMF (20 mL) was added HATU (2.67 g, 7.0 mmol) followed by DIPEA (3.02 g, 23.4 mmol) dropwise. The resulting mixture was stirred at rt for 1 h at which point it was diluted with water (50 mL) and the product was extracted with EtOAc (3×30 mL). The organic layers were collected and combined then washed with brine, dried over Na₂SO₄, and concentrated in vacuo. The resulting crude material was purified by silica gel chromatography (1:1 EtOAc/Pet. Ether) fractions containing product were combined and concentrated to afford the product as a light-yellow solid (0.880 g, 39% yield).

ESI-MS m/ [symbol] : 470.8 [M+H]⁺

Separation of diastereomers was done at this step using reverse phase chromatography: Column: Xselect CSH F-Phenyl OBD, 19*250, Sum; 45% to 60% MeOH/water (0.1% FA) in 5 min; Flow rate: 25 mL/min.

Peak 1_D1 contained 270 mg of a white solid.

Peak 2_D2 Contained 259 mg of a white solid.

Enantiomers of this material were separated by Prep-chiral-HPLC:

D1: Column: CHIRAL ART Cellulose-SB, 2*25 cm, 5 m; Mobile Phase A: Hex:MTBE=1:1 (0.5% 2M NH3-MeOH), Mobile Phase B:EtOH-HPLC; Flow rate: 20 mL/min; Gradient: 30% B to 30% B in 9.5 min

Peak 1 (Isomer-1_D1E1): R_{T} 6.38 min; afforded a white solid (131 mg)

Peak 2 (Isomer-2_D1E2): R_{T} 7.49 min; afforded a white solid (132 mg)

D2: CHIRAL ART Cellulose-SB, 2*25 cm, 5 m; Mobile Phase A: Hex:MTBE=1:1 (0.5% 2M NH3-MeOH), Mobile Phase B:EtOH-HPLC; Flow rate: 20 mL/min; Gradient: 25% B to 25% B in 10 min

Peak 1 (Isomer-3_D2E1): R_{T} 5.64 min; afforded a white solid (101 mg).

Peak 2 (Isomer-4_D2E2): R_{T} 6.43 min; afforded a white solid (106 mg).

### Step 6: 2-[1-(2-cyanopyridin-3-yl)-1-phenylpropan-2-yl]-5-hydroxy-1-methyl-N-(1,2-oxazol-4-yl)-6-oxopyrimidine-4-carboxamide

To a solution of (2-[1-(2-cyanopyridin-3-yl)-1-phenylpropan-2-yl]-5-methoxy-1-methyl-N-(1,2-oxazol-4-yl)-6-oxopyrimidine-4-carboxamide (0.131 g, 0.28 mmol) dissolved in DMF (6.5 ml) was added LiBr (0.484 g, 5.6 mmol). This resulting mixture was then heated to 95° C. and stirred for 1 h at which point complete conversion to the product was observed by LCMS. The reaction was then cooled to rt and concentrated in vacuo. The resulting crude material was purified by reverse phase chromatography.

Isomer-1_D1E1: Isolated a white solid (0.052 g, 40% yield).

ESI-MS m/ [symbol] : 457.0 [M+H]⁺; >98% ee

¹H NMR (400 MHz, DMSO-d₆) δ 11.10 (s, 1H), 10.58 (s, 1H), 9.30 (s, 1H), 8.87 (s, 1H), 8.42-8.40 (m, 2H), 7.73-7.71 (m, 2H), 7.60 (dd, 1H), 7.44 (t, 2H), 7.41-7.28 (m, 1H), 5.26 (d, 1H), 4.28 (dq, 1H), 3.68 (s, 3H), 1.25 (d, 3H).

Isomer-2_D1E2: Isolated a white solid (0.068 g, 53% yield)

ESI-MS m/ [symbol] : 457.0 [M+H]⁺; >98% ee

¹H NMR (400 MHz, DMSO-d6): δ 11.10 (s, 1H), 10.56 (s, 1H), 9.31 (s, 1H), 8.87 (s, 1H), 8.42-8.40 (m, 2H), 7.73-7.71 (m, 2H), 7.60 (m, 1H), 7.43 (t, 2H), 7.32-7.28 (m, 1H), 5.26 (d, 1H), 4.28 (m, 1H), 3.69 (s, 3H), 1.25 (d, 3H).

Isomer-3_D2E1: Isolated a white solid (0.041 g, 47% yield)

ESI-MS m/ [symbol] : 457.1 [M+H]⁺; >98% ee

¹H NMR (400 MHz, DMSO-d6 δ 11.19 (s, 1H), 10.60 (s, 1H), 9.34 (s, 1H), 8.92 (s, 1H), 8.71-8.67 (m, 2H), 7.87 (m, 1H), 7.24-7.17 (m, 4H), 7.11-7.08 (m, 1H), 4.99 (d, 1H), 4.33 (m, 1H), 3.43 (s, 3H), 1.30 (d, 3H).

Isomer-4_D2E2: Isolated a white solid (0.049 g, 47% yield)

ESI-MS m/ [symbol] : 457.1 [M+H]⁺; >98% ee

¹H NMR (400 MHz, DMSO-d6): δ 11.20 (s, 1H), 10.60 (s, 1H), 9.35 (s, 1H), 8.92 (s, 1H), 8.69-8.67 (m, 2H), 7.87 (dd, 1H), 7.24-7.17 (m, 4H), 7.10 (t, 1H), 4.98 (d, 1H), 4.34 (dd, 1H), 3.43 (s, 3H), 1.30 (d, 3H).

### Example 156

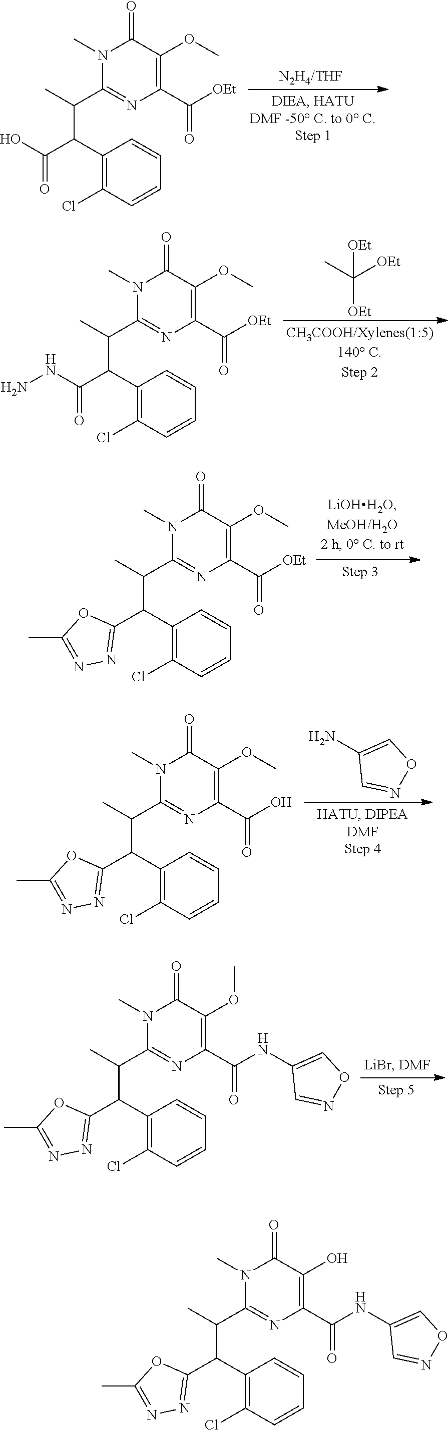

D1 isomer was obtained using t-Bu protected carboxylic acid

D2 Isomer was obtained using Bn protected carboxylic acid

### Step 1: ethyl 2-[1-(2-chlorophenyl)-1-(hydrazinecarbonyl)propan-2-yl]-5-methoxy-1-methyl-6-oxopyrimidine-4-carboxylate

To a stirred mixture of 2-(2-chlorophenyl)-3-[4-(ethoxycarbonyl)-5-methoxy-1-methyl-6-oxopyrimidin-2-yl]butanoic acid (0.200 g, 0.5 mmol) and N,N,N,N-Tetramethyl-O-(7-azabenzotriazol-1-yl)uronium hexafluorophospate (0.465 g, 1.2 mmol) in DMF (2 ml) was added hydrazine (1.00 mL, 20.6 mmol) dropwise at −5° C. followed by DIPEA (189.67 mg, 1.5 mmol). The resulting mixture was stirred for 20 min at −5° C. and then warmed to 0° C. and quenched with water. Product was extracted with EtOAc (3×100 mL) and the combined organic layers were washed with water (3×100 mL), dried over Na₂SO₄, and concentrated in vacuo. The crude product was used in the next step directly without further purification.

ESI-MS m/z 423.0 [M+H]⁺.

### Step 2: ethyl 2-[1-(2-chlorophenyl)-1-(5-methyl-1,3,4-oxadiazol-2-yl) propan-2-yl]-5-methoxy-1-methyl-6-oxopyrimidine-4-carboxylate

Into a 40 mL vial were added ethyl 2-[1-(2-chlorophenyl)-1-(hydrazinecarbonyl)propan-2-yl]-5-methoxy-1-methyl-6-oxopyrimidine-4-carboxylate (1.22 g, 2.885 mmol) and triethyl orthoacetate (0.94 mg, 0.006 mmol) followed by a mixture of acetic acid and Xylenes (1:6) (7.00 mL). The resulting mixture was heated to 140° C. and stirred for 1 h then cooled to rt and dried in vacuo. The crude product was purified by reverse phase chromatography to afford the product as a dark yellow oil (0.300 g, 25% yield).

ESI-MS m/z 447.1 [M+H]⁺.

### Step 3 2-[1-(2-chlorophenyl)-1-(5-methyl-1,3,4-oxadiazol-2-yl)propan-2-yl]-5-methoxy-1-methyl-6-oxopyrimidine-4-carboxylic acid

To a stirred solution of 3(ethyl 2-[1-(2-chlorophenyl)-1-(1,3,4-oxadiazol-2-yl)propan-2-yl]-5-methoxy-1-methyl-6-oxopyrimidine-4-carboxylate (0.300 g, 1.2 mmol) in MeOH/water (5:1, 5 mL) was added LiOH·H₂O (0.056 g, 2.3 mmol). The reaction mixture was stirred at rt for 2 h at which point the mixture was concentrated in vacuo and the resulting product was used directly without further purification

ESI-MS m/z 419.1 [M+H]⁺.

### Step 4 5(2-[1-(2-chlorophenyl)-1-(5-methyl-1,3,4-oxadiazol-2-yl)propan-2-yl]-5-methoxy-1-methyl-N-(1,2-oxazol-4-yl)-6-oxopyrimidine-4-carboxamide

To a stirred solution of 2-[1-(2-chlorophenyl)-1-(5-methyl-1,3,4-oxadiazol-2-yl)propan-2-yl]-5-methoxy-1-methyl-6-oxopyrimidine-4-carboxylic acid (0.385 g, 0.9 mmol) and 1,2-oxazol-4-amine hydrochloride (0.116 g, 1.4 mmol) in DMF (4.5 mL) was added HATU (0.699 g, 1.8 mmol) followed by DIPEA (0.594 g, 4.6 mmol) dropwise. The resulting mixture was stirred at rt for 1 h at which point it was diluted with water (50 mL) and the product was extracted with EtOAc (3×30 mL). The organic layers were collected and combined then washed with brine, dried over Na₂SO₄, and concentrated in vacuo. The resulting crude material was purified by silica gel chromatography (1:1 EtOAc/Pet. Ether) fractions containing product were combined and concentrated to afford the product as a dark yellow solid (0.300 g, 78% yield).

ESI-MS m/ [symbol] : 485.1 [M+H]⁺

Enantiomers of this material were separated by Prep-chiral-HPLC:

D1: Column: CHIRALPAK IC-3, 4.6*50 mm 3 um; Mobile Phase A: Hex:MTBE 1:1 (0.1% DEA), Mobile Phase B:EtOH-HPLC; Flow rate: 1 mL/min; Gradient: 30% B to 30% B

Peak 1 (Isomer-1_D1E1): afforded a white solid (158 mg)

Peak 2 (Isomer-2_D1E2): afforded a white solid (108 mg)

D2: CHIRALPAK IF-3, 4.6*50 mm, 3.0 um; Mobile Phase A: Hex:MTBE=1:1 (0.5% 2M NH3-MeOH), Mobile Phase B:EtOH-HPLC; Flow rate: 20 mL/min; Gradient: 25% B to 25% B in 10 min

Peak 1 (Isomer-3_D2E1): afforded a white solid (96 mg).

Peak 2 (Isomer-4_D2E2): afforded a white solid (93 mg).

### Step 5 2-(1-(2-chlorophenyl)-1-(5-methyl-1,3,4-oxadiazol-2-yl)propan-2-yl)-5-hydroxy-N-(isoxazol-4-yl)-1-methyl-6-oxo-1,6-dihydropyrimidine-4-carboxamide

To a solution of 2-[1-(2-chlorophenyl)-1-(5-methyl-1,3,4-oxadiazol-2-yl)propan-2-yl]-5-methoxy-1-methyl-N-(1,2-oxazol-4-yl)-6-oxopyrimidine-4-carboxamide (0.096 g, 0.2 mmol) dissolved in DMF (5.0 ml) was added LiBr (0.258 g, 3 mmol). This resulting mixture was then heated to 95° C. and stirred for 3 h at which point complete conversion to the product was observed by LCMS. The reaction was then cooled to rt and concentrated in vacuo. The resulting crude material was purified by reverse phase chromatography.

Isomer-1_D1E1: Isolated a white solid

ESI-MS m/ [symbol] : 471.1 [M+H]⁺; >98% ee

¹H NMR (400 MHz, DMSO-d₆) δ 11.36 (s, 1H), 10.50 (s, 1H), 9.31 (s, 1H), 8.89 (s, 1H), 7.63 (d, J=7.4 Hz, 1H), 7.55 (dd, J=7.6, 1.7 Hz, 1H), 7.48-7.38 (m, 2H), 5.49 (d, J=10.8 Hz, 1H), 4.13-4.08 (m, 1H), 3.73 (s, 3H), 2.32 (s, 3H), 1.10 (d, J=6.8 Hz, 3H)

Isomer-2_D1E2: Isolated a white solid

ESI-MS m/ [symbol] : 471.1 [M+H]⁺; >98% ee

¹H NMR (400 MHz, DMSO-d₆) δ 11.36 (s, 1H), 10.49 (s, 1H), 9.31 (s, 1H), 8.89 (s, 1H), 7.63 (d, J=7.5 Hz, 1H), 7.55 (dd, J=7.6, 1.7 Hz, 1H), 7.48-7.27 (m, 2H), 5.49 (d, J=10.8 Hz, 1H), 4.13-4.08 (m, 1H), 3.73 (s, 2H), 2.32 (s, 2H), 1.10 (d, J=6.8 Hz, 2H).

Isomer-3_D2E1: Isolated a white solid (0.041 g, 47% yield)

ESI-MS m/ [symbol] : 471.2 [M+H]⁺; >98% ee

¹H NMR (400 MHz, Chloroform-d) δ 11.42 (s, 1H), 9.46 (s, 1H), 9.11 (s, 1H), 8.72 (s, 1H), 7.43 (dd, J=7.7, 1.8 Hz, 1H), 7.37 (dd, J=7.8, 1.5 Hz, 1H), 7.28-7.18 (m, 2H), 5.54 (d, J=9.1 Hz, 1H), 4.10-4.02 (m, 1H), 3.69 (s, 3H), 2.55 (s, 3H), 1.45 (d, J=6.7 Hz, 3H).

Isomer-4_D2E2: Isolated a white solid (0.049 g, 47% yield)

ESI-MS m/ [symbol] : 471.2 [M+H]⁺; >95% ee

¹H NMR (400 MHz, Chloroform-d) δ 11.42 (s, 1H), 9.47 (s, 1H), 9.11 (s, 1H), 8.72 (s, 1H), 7.43 (dd, J=7.7, 1.8 Hz, 1H), 7.37 (dd, J=7.8, 1.5 Hz, 1H), 7.27-7.19 (m, 2H), 5.54 (d, J=9.1 Hz, 1H), 4.10-4.02 (m, 1H), 3.69 (s, 3H), 2.55 (s, 3H), 1.45 (d, J=6.8 Hz, 3H).

TABLE 6
Example numbers and chemical names of all isomers

| Example | Names |
|---|---|
| 1 | 2-((1S,2R)-1-(2-cyanophenyl)-1-(1-methyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-methyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-methyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-methyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| 2 | 2-((1S,2R)-1-(2-cyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 3 | 2-((1S,2R)-1-(2-cyanophenyl)-1-(1,3-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,3-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,3-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,3-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 4 | 2-((1S,2R)-1-(2-cyanophenyl)-1-(1,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 5 | 2-((1S,2R)-1-(2-cyanophenyl)-1-(6-methylpyridin-3-yl)propan-2-yl)-5-hydroxy- |
| | N-(isoxazol-4-yl)-1-methyl-6-oxo-1,6-dihydropyrimidine-4-carboxamide |
| | 2-((1R,2S)-1-(2-cyanophenyl)-1-(6-methylpyridin-3-yl)propan-2-yl)-5-hydroxy- |
| | N-(isoxazol-4-yl)-1-methyl-6-oxo-1,6-dihydropyrimidine-4-carboxamide |
| | 2-((1R,2R)-1-(2-cyanophenyl)-1-(6-methylpyridin-3-yl)propan-2-yl)-5-hydroxy- |
| | N-(isoxazol-4-yl)-1-methyl-6-oxo-1,6-dihydropyrimidine-4-carboxamide |
| | 2-((1S,2S)-1-(2-cyanophenyl)-1-(6-methylpyridin-3-yl)propan-2-yl)-5-hydroxy- |
| | N-(isoxazol-4-yl)-1-methyl-6-oxo-1,6-dihydropyrimidine-4-carboxamide |
| 6 | 2-((1R,2R)-1-(2-cyanophenyl)-1-(2-methylpyridin-4-yl)propan-2-yl)-5-hydroxy- |
| | N-(isoxazol-4-yl)-1-methyl-6-oxo-1,6-dihydropyrimidine-4-carboxamide |
| | 2-((1S,2S)-1-(2-cyanophenyl)-1-(2-methylpyridin-4-yl)propan-2-yl)-5-hydroxy- |
| | N-(isoxazol-4-yl)-1-methyl-6-oxo-1,6-dihydropyrimidine-4-carboxamide |
| | 2-((1S,2R)-1-(2-cyanophenyl)-1-(2-methylpyridin-4-yl)propan-2-yl)-5-hydroxy- |
| | N-(isoxazol-4-yl)-1-methyl-6-oxo-1,6-dihydropyrimidine-4-carboxamide |
| | 2-((1R,2S)-1-(2-cyanophenyl)-1-(2-methylpyridin-4-yl)propan-2-yl)-5-hydroxy- |
| | N-(isoxazol-4-yl)-1-methyl-6-oxo-1,6-dihydropyrimidine-4-carboxamide |
| 7 | 2-((1R,2R)-1-(2-cyanopyridin-3-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanopyridin-3-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanopyridin-3-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanopyridin-3-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 8 | 2-((1R,2R)-1-(3-cyanopyridin-4-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(3-cyanopyridin-4-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(3-cyanopyridin-4-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(3-cyanopyridin-4-yl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 9 | 2-((1S,2R)-1-(2-cyanophenyl)-1-(1-(2-methoxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(2-methoxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-(2-methoxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(2-methoxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 10 | 2-((1R,2R)-1-(3-cyano-1-methyl-1H-pyrazol-4-yl)-1-(2-cyanophenyl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(3-cyano-1-methyl-1H-pyrazol-4-yl)-1-(2-cyanophenyl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(3-cyano-1-methyl-1H-pyrazol-4-yl)-1-(2-cyanophenyl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(3-cyano-1-methyl-1H-pyrazol-4-yl)-1-(2-cyanophenyl)propan-2- |
| | yl)-5-hydroxy-N-(isoxazol-4-yl)-1-methyl-6-oxo-1,6-dihydropyrimidine-4- |
| | carboxamide |
| 11 | 2-((1R,2R)-1-(2-cyano-4-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-4-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-4-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-4-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 12 | 2-((1S,2R)-1-(2-cyanophenyl)-1-(1H-pyrazol-4-yl)propan-2-yl)-5-hydroxy-N- |
| | (isoxazol-4-yl)-1-methyl-6-oxo-1,6-dihydropyrimidine-4-carboxamide |
| | 2-((1R,2S)-1-(2-cyanophenyl)-1-(1H-pyrazol-4-yl)propan-2-yl)-5-hydroxy-N- |
| | (isoxazol-4-yl)-1-methyl-6-oxo-1,6-dihydropyrimidine-4-carboxamide |
| | 2-((1R,2R)-1-(2-cyanophenyl)-1-(1H-pyrazol-4-yl)propan-2-yl)-5-hydroxy-N- |
| | (isoxazol-4-yl)-1-methyl-6-oxo-1,6-dihydropyrimidine-4-carboxamide |
| | 2-((1S,2S)-1-(2-cyanophenyl)-1-(1H-pyrazol-4-yl)propan-2-yl)-5-hydroxy-N- |
| | (isoxazol-4-yl)-1-methyl-6-oxo-1,6-dihydropyrimidine-4-carboxamide |
| 13 | 2-((1S,2R)-1-(2-cyanophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 14 | 2-((1R,2R)-1-(2-cyano-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 15 | 2-((1R,2R)-1-(2-cyano-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyano-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyano-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 16 | 2-((1R,2R)-1-(2-cyano-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-4-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyano-4-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyano-4-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 17 | 2-((1R,2R)-1-(2-cyano-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 18 | 2-((1R,2R)-1-(2-cyano-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 19 | 2-((1S,2S)-1-(2,6-dicyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2,6-dicyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2,6-dicyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2,6-dicyanophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 20 | 2-((1S,2R)-1-(2-cyanophenyl)-1-(1-(2-morpholinoethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(2-morpholinoethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-(2-morpholinoethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(2-morpholinoethyl)-1l4,2l2-pyrazol-4- |
| | yl)propan-2-yl)-5-hydroxy-N-(isoxazol-4-yl)-1-methyl-6-oxo-1,6- |
| | dihydropyrimidine-4-carboxamide |
| 21 | tert-butyl 4-(2-(4-((1S,2R)-1-(2-cyanophenyl)-2-(5-hydroxy-4-(isoxazol-4- |
| | ylcarbamoyl)-1-methyl-6-oxo-1,6-dihydropyrimidin-2-yl)propyl)-1H-pyrazol-1- |
| | yl)ethyl)piperazine-1-carboxylate |
| | tert-butyl 4-(2-(4-((1R,2S)-1-(2-cyanophenyl)-2-(5-hydroxy-4-(isoxazol-4- |
| | ylcarbamoyl)-1-methyl-6-oxo-1,6-dihydropyrimidin-2-yl)propyl)-1H-pyrazol-1- |
| | yl)ethyl)piperazine-1-carboxylate |
| | tert-butyl 4-(2-(4-((1S,2S)-1-(2-cyanophenyl)-2-(5-hydroxy-4-(isoxazol-4- |
| | ylcarbamoyl)-1-methyl-6-oxo-1,6-dihydropyrimidin-2-yl)propyl)-1H-pyrazol-1- |
| | yl)ethyl)piperazine-1-carboxylate |
| | tert-butyl 4-(2-(4-((1R,2R)-1-(2-cyanophenyl)-2-(5-hydroxy-4-(isoxazol-4- |
| | ylcarbamoyl)-1-methyl-6-oxo-1,6-dihydropyrimidin-2-yl)propyl)-1H-pyrazol-1- |
| | yl)ethyl)piperazine-1-carboxylate |
| 22 | 2-((1S,2R)-1-(2-cyanophenyl)-1-(1-(2,2,2-trifluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2,2,2-trifluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2,2,2-trifluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(2,2,2-trifluoroethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 23 | 2-((1R,2R)-1-(2-cyanophenyl)-1-(1-(cyclopropylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(cyclopropylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-(cyclopropylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(cyclopropylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 24 | 2-((1R,2R)-1-(2-cyanophenyl)-1-(1-(3-methoxypropyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(3-methoxypropyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(3-methoxypropyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-(3-methoxypropyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 25 | 2-((1R,2R)-1-(2-cyano-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyano-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyano-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyano-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 26 | 2-((1R,2R)-1-(5-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(5-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(5-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(5-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 27 | 2-((1R,2R)-1-(4-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(4-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(4-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(4-chloro-2-cyanophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 28 | 2-((1S,2R)-1-(2-chloro-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-chloro-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(2-chloro-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-chloro-4,5-difluorophenyl)-1-(1-methyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 29 | 2-((1R,2R)-1-(2-cyano-5-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 30 | 2-((1S,2S)-1-(2-cyano-4-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyano-4-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-4-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-4-(trifluoromethyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 31 | 2-((1S,2R)-1-(2-cyanophenyl)-1-(1-(2-(piperazin-1-yl)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-(piperazin-1-yl)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(2-(piperazin-1-yl)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-(piperazin-1-yl)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 32 | 2-((1S,2R)-1-(2-cyanophenyl)-1-(1-(2-(4-methylpiperazin-1-yl)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-(4-methylpiperazin-1-yl)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(2-(4-methylpiperazin-1-yl)ethyl)-1H-pyrazol- |
| | 4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-(4-methylpiperazin-1-yl)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 33 | 2-((1S,2R)-1-(2-cyanophenyl)-1-(1-(2-(dimethylamino)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-(dimethylamino)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(2-(dimethylamino)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-(dimethylamino)ethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 34 | 2-((1R,2S)-1-(2-cyanophenyl)-1-(1-(3-(dimethylamino)propyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-(3-(dimethylamino)propyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(3-(dimethylamino)propyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(3-(dimethylamino)propyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 35 | 2-((1S,2R)-1-(2-cyanophenyl)-1-(1-(2-hydroxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(2-hydroxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-(2-hydroxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(2-hydroxyethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 36 | 2-((1R,2R)-1-(2-cyanophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 37 | 2-((1S,2S)-1-(2-cyanophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 38 | 2-((1R,2R)-1-(2-cyanophenyl)-1-(1-(difluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(difluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-(difluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(difluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 39 | 2-((1R,2R)-1-(2-cyanophenyl)-1-(1-methyl-1H-imidazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-methyl-1H-imidazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-methyl-1H-imidazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-methyl-1H-imidazol-4-yl)propan-2-yl)-5- |
| | hydroxy-N-(isoxazol-4-yl)-1-methyl-6-oxo-1,6-dihydropyrimidine-4- |
| | carboxamide |
| 40 | 2-((2R,3S)-3-(2-cyanophenyl)-1,1,1-trifluoro-3-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((2S,3R)-3-(2-cyanophenyl)-1,1,1-trifluoro-3-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((2S,3S)-3-(2-cyanophenyl)-1,1,1-trifluoro-3-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((2R,3R)-3-(2-cyanophenyl)-1,1,1-trifluoro-3-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 41 | 2-((2R,3S)-3-(2-cyanophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2S,3R)-3-(2-cyanophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2S,3S)-3-(2-cyanophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2R,3R)-3-(2-cyanophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| 42 | 2-((2S,3S)-3-(2-cyano-5-fluorophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2R,3R)-3-(2-cyano-5-fluorophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2S,3R)-3-(2-cyano-5-fluorophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| | 2-((2R,3S)-3-(2-cyano-5-fluorophenyl)-3-(1,3-dimethyl-1H-pyrazol-4-yl)-1,1,1- |
| | trifluoropropan-2-yl)-5-hydroxy-N-(isoxazol-4-yl)-1-methyl-6-oxo-1,6- |
| | dihydropyrimidine-4-carboxamide |
| 148 | (R)-2-(2-(2-cyanophenyl)-1,1-difluoro-2-(1-methyl-1H-pyrazol-4-yl)ethyl)-5- |
| | hydroxy-N-(isoxazol-4-yl)-1-methyl-6-oxo-1,6-dihydropyrimidine-4- |
| | carboxamide |
| | (S)-2-(2-(2-cyanophenyl)-1,1-difluoro-2-(1-methyl-1H-pyrazol-4-yl)ethyl)-5- |
| | hydroxy-N-(isoxazol-4-yl)-1-methyl-6-oxo-1,6-dihydropyrimidine-4- |
| | carboxamide |
| 44 | 2-((1R,2R)-1-(2-chlorophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| 45 | 2-((1S,2R)-1-(2-chlorophenyl)-1-(1H-pyrazol-1-yl)propan-2-yl)-5-hydroxy-N- |
| | (isoxazol-4-yl)-1-methyl-6-oxo-1,6-dihydropyrimidine-4-carboxamide |
| | 2-((1R,2S)-1-(2-chlorophenyl)-1-(1H-pyrazol-1-yl)propan-2-yl)-5-hydroxy-N- |
| | (isoxazol-4-yl)-1-methyl-6-oxo-1,6-dihydropyrimidine-4-carboxamide |
| | 2-((1S,2S)-1-(2-chlorophenyl)-1-(1H-pyrazol-1-yl)propan-2-yl)-5-hydroxy-N- |
| | (isoxazol-4-yl)-1-methyl-6-oxo-1,6-dihydropyrimidine-4-carboxamide |
| | 2-((1R,2R)-1-(2-chlorophenyl)-1-(1H-pyrazol-1-yl)propan-2-yl)-5-hydroxy-N- |
| | (isoxazol-4-yl)-1-methyl-6-oxo-1,6-dihydropyrimidine-4-carboxamide |
| 46 | 2-((1R,2R)-1-(2-cyanophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1S,2S)-1-(2-cyanophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1R,2S)-1-(2-cyanophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1S,2R)-1-(2-cyanophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| 47 | 2-((1S,2S)-1-(2-cyanophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 48 | 2-((1S,2R)-1-(3-cyano-1-methyl-1H-pyrazol-4-yl)-1-(2,5-difluorophenyl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(3-cyano-1-methyl-1H-pyrazol-4-yl)-1-(2,5-difluorophenyl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(3-cyano-1-methyl-1H-pyrazol-4-yl)-1-(2,5-difluorophenyl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(3-cyano-1-methyl-1H-pyrazol-4-yl)-1-(2,5-difluorophenyl)propan- |
| | 2-yl)-5-hydroxy-N-(isoxazol-4-yl)-1-methyl-6-oxo-1,6-dihydropyrimidine-4- |
| | carboxamide |
| 49 | 2-((1R,2R)-1-(2-chlorophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 50 | 2-((1S,2R)-1-(2-cyanophenyl)-1-(oxazol-4-yl)propan-2-yl)-5-hydroxy-N- |
| | (isoxazol-4-yl)-1-methyl-6-oxo-1,6-dihydropyrimidine-4-carboxamide |
| | 2-((1R,2S)-1-(2-cyanophenyl)-1-(oxazol-4-yl)propan-2-yl)-5-hydroxy-N- |
| | (isoxazol-4-yl)-1-methyl-6-oxo-1,6-dihydropyrimidine-4-carboxamide |
| | 2-((1S,2S)-1-(2-cyanophenyl)-1-(oxazol-4-yl)propan-2-yl)-5-hydroxy-N- |
| | (isoxazol-4-yl)-1-methyl-6-oxo-1,6-dihydropyrimidine-4-carboxamide |
| | 2-((1R,2R)-1-(2-cyanophenyl)-1-(oxazol-4-yl)propan-2-yl)-5-hydroxy-N- |
| | (isoxazol-4-yl)-1-methyl-6-oxo-1,6-dihydropyrimidine-4-carboxamide |
| 51 | 2-((1S,2R)-1-(2-cyanophenyl)-1-(1-methyl-1H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-methyl-1H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-methyl-1H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-methyl-1H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| 52 | 2-((1R,2R)-1-(4-cyano-2-methyl-1l2,2l4-pyrazol-3-yl)-1-(2-cyanophenyl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(4-cyano-2-methyl-1l2,2l4-pyrazol-3-yl)-1-(2-cyanophenyl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(4-cyano-2-methyl-1l2,2l4-pyrazol-3-yl)-1-(2-cyanophenyl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(4-cyano-2-methyl-1l2,2l4-pyrazol-3-yl)-1-(2-cyanophenyl)propan- |
| | 2-yl)-5-hydroxy-N-(isoxazol-4-yl)-1-methyl-6-oxo-1,6-dihydropyrimidine-4- |
| | carboxamide |
| 53 | 2-((1S,2R)-1-(2-cyanophenyl)-1-(6-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1R,2S)-1-(2-cyanophenyl)-1-(6-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1S,2S)-1-(2-cyanophenyl)-1-(6-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1R,2R)-1-(2-cyanophenyl)-1-(6-methylpyrazin-2-yl)propan-2-yl)-5-hydroxy- |
| | N-(isoxazol-4-yl)-1-methyl-6-oxo-1,6-dihydropyrimidine-4-carboxamide |
| 54 | 2-((1S,2S)-1-(2-chlorophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 55 | 2-((1S,2R)-1-(2-cyano-4-fluorophenyl)-1-(1-ethyl-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-4-fluorophenyl)-1-(1-ethyl-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-4-fluorophenyl)-1-(1-ethyl-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyano-4-fluorophenyl)-1-(1-ethyl-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 56 | 2-((1S,2R)-1-(2-cyano-4-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyano-4-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyano-4-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(2-cyano-4-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 57 | 2-((1R,2R)-1-(2-cyano-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 58 | 2-((1S,2R)-1-(2-chlorophenyl)-1-(3-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(3-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(3-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(3-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 59 | 2-((1R,2R)-1-(3-cyano-1-methyl-1H-pyrazol-4-yl)-1-(2-cyano-5- |
| | fluorophenyl)propan-2-yl)-5-hydroxy-N-(isoxazol-4-yl)-1-methyl-6-oxo-1,6- |
| | dihydropyrimidine-4-carboxamide |
| | 2-((1S,2S)-1-(3-cyano-1-methyl-1H-pyrazol-4-yl)-1-(2-cyano-5- |
| | fluorophenyl)propan-2-yl)-5-hydroxy-N-(isoxazol-4-yl)-1-methyl-6-oxo-1,6- |
| | dihydropyrimidine-4-carboxamide |
| | 2-((1S,2R)-1-(3-cyano-1-methyl-1H-pyrazol-4-yl)-1-(2-cyano-5- |
| | fluorophenyl)propan-2-yl)-5-hydroxy-N-(isoxazol-4-yl)-1-methyl-6-oxo-1,6- |
| | dihydropyrimidine-4-carboxamide |
| | 2-((1R,2S)-1-(3-cyano-1-methyl-1H-pyrazol-4-yl)-1-(2-cyano-5- |
| | fluorophenyl)propan-2-yl)-5-hydroxy-N-(isoxazol-4-yl)-1-methyl-6-oxo-1,6- |
| | dihydropyrimidine-4-carboxamide |
| 60 | 2-((1S,2R)-1-(2-chloro-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2R)-1-(2-chloro-5-fluorophenyl)-1-(1-ethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 61 | 2-((1S,2R)-1-(2-chloro-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chloro-5-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 62 | 2-((1S,2R)-1-(2-chloro-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(2-chloro-5-fluorophenyl)-1-(1,3-dimethyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| 63 | 2-((1S,2R)-1-(2-cyanophenyl)-1-(1,3-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,3-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,3-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,3-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| 64 | 2-((1S,2R)-1-(2-cyanophenyl)-1-(1,4-dimethyl-1H-pyrazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,4-dimethyl-1H-pyrazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,4-dimethyl-1H-pyrazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,4-dimethyl-1H-pyrazol-3-yl)propan-2-yl)-5- |
| | hydroxy-N-(isoxazol-4-yl)-1-methyl-6-oxo-1,6-dihydropyrimidine-4- |
| | carboxamide |
| 65 | 2-((1S,2R)-1-(2-cyano-4-fluorophenyl)-1-(1-ethyl-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-4-fluorophenyl)-1-(1-ethyl-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-4-fluorophenyl)-1-(1-ethyl-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyano-4-fluorophenyl)-1-(1-ethyl-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 66 | 2-((1S,2R)-1-(2-cyanophenyl)-1-(pyrazin-2-yl)propan-2-yl)-5-hydroxy-N- |
| | (isoxazol-4-yl)-1-methyl-6-oxo-1,6-dihydropyrimidine-4-carboxamide |
| | 2-((1R,2S)-1-(2-cyanophenyl)-1-(pyrazin-2-yl)propan-2-yl)-5-hydroxy-N- |
| | (isoxazol-4-yl)-1-methyl-6-oxo-1,6-dihydropyrimidine-4-carboxamide |
| | 2-((1S,2S)-1-(2-cyanophenyl)-1-(pyrazin-2-yl)propan-2-yl)-5-hydroxy-N- |
| | (isoxazol-4-yl)-1-methyl-6-oxo-1,6-dihydropyrimidine-4-carboxamide |
| | 2-((1R,2R)-1-(2-cyanophenyl)-1-(pyrazin-2-yl)propan-2-yl)-5-hydroxy-N- |
| | (isoxazol-4-yl)-1-methyl-6-oxo-1,6-dihydropyrimidine-4-carboxamide |
| 67 | 2-((1S,2R)-1-(2-cyanophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(2-methylpyrimidin-5-yl)propan-2-yl)-5- |
| | hydroxy-N-(isoxazol-4-yl)-1-methyl-6-oxo-1,6-dihydropyrimidine-4- |
| | carboxamide |
| 68 | 2-((1R,2R)-1-(2-cyanophenyl)-1-(1-ethyl-5-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-ethyl-5-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-ethyl-5-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-ethyl-5-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 69 | 2-((1S,2S)-1-(2-cyanophenyl)-1-(1-(2-methoxyethyl)-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-methoxyethyl)-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-methoxyethyl)-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-(2-methoxyethyl)-5-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 70 | 2-((1R,2R)-1-(2-cyanophenyl)-1-(1-ethyl-3-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-ethyl-3-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-ethyl-3-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-ethyl-3-methyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 71 | 2-((1S,2S)-1-(2-cyanophenyl)-1-(1-(2-methoxyethyl)-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-cyanophenyl)-1-(1-(2-methoxyethyl)-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-(2-methoxyethyl)-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-(2-methoxyethyl)-3-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 72 | 2-((1S,2R)-1-(2-chloro-4,5-difluorophenyl)-1-(1-ethyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-4,5-difluorophenyl)-1-(1-ethyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chloro-4,5-difluorophenyl)-1-(1-ethyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-4,5-difluorophenyl)-1-(1-ethyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 73 | 2-((1S,2S)-1-(2-chloro-4,5-difluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-4,5-difluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-4,5-difluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-chloro-4,5-difluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 74 | 2-((1S,2R)-1-(2-chloro-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chloro-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-4-fluorophenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 75 | 2-((1R,2R)-1-(2-cyano-5-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-(dimethylcarbamoyl)phenyl)-1-(1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 76 | 2-((1R,2R)-1-(2-cyanophenyl)-1-(1-(methyl-d3)-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-(1-(2-cyanophenyl)-1-(1-(methyl-d3)-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-(1-(2-cyanophenyl)-1-(1-(methyl-d3)-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-(1-(2-cyanophenyl)-1-(1-(methyl-d3)-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 77 | 2-((1S,2S)-1-(2-chlorophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(1-methyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 78 | 2-((1S,2R)-1-(2-cyanophenyl)-1-(1,3,5-trimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,3,5-trimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,3,5-trimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,3,5-trimethyl-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 79 | 2-((1S,2R)-1-(2-cyanophenyl)-1-(2-methyl-2H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(2-methyl-2H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(2-methyl-2H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(2-methyl-2H-tetrazol-5-yl)propan-2-yl)-5- |
| | hydroxy-N-(isoxazol-4-yl)-1-methyl-6-oxo-1,6-dihydropyrimidine-4- |
| | carboxamide |
| 80 | 2-((1R,2R)-1-(2-cyano-4-hydroxyphenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-4-hydroxyphenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyano-4-hydroxyphenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyano-4-hydroxyphenyl)-1-(1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 81 | 2-((1S,2R)-1-(2-cyanophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 82 | 2-((1S,2R)-1-(2-cyanophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 83 | 2-((1S,2R)-1-(2-cyanophenyl)-1-(2-methylpyrimidin-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(2-methylpyrimidin-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(2-methylpyrimidin-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(2-methylpyrimidin-4-yl)propan-2-yl)-5- |
| | hydroxy-N-(isoxazol-4-yl)-1-methyl-6-oxo-1,6-dihydropyrimidine-4- |
| | carboxamide |
| 84 | 2-((1S,2R)-1-(2-cyanophenyl)-1-(1,4-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1,4-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1,4-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1,4-dimethyl-1H-pyrazol-5-yl)propan-2-yl)-5- |
| | hydroxy-N-(isoxazol-4-yl)-1-methyl-6-oxo-1,6-dihydropyrimidine-4- |
| | carboxamide |
| 85 | 2-((1S,2R)-1-(2-chlorophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(1-ethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 86 | 2-((1S,2R)-1-(2-chlorophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chlorophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chlorophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chlorophenyl)-1-(1-(2-hydroxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 87 | 2-((1S,2R)-1-(2-chlorophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chlorophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chlorophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chlorophenyl)-1-(1-(2-methoxy-2-methylpropyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 88 | 2-((1S,2R)-1-(2-chlorophenyl)-1-(1-(oxetan-3-ylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(1-(oxetan-3-ylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(1-(oxetan-3-ylmethyl)-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(1-(oxetan-3-ylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 89 | 2-((1S,2R)-1-(2-chloro-5-fluorophenyl)-1-(3-cyano-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(3-cyano-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(3-cyano-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(3-cyano-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 90 | 2-((1R,2R)-1-(2-cyanophenyl)-1-(1-methyl-3-(trifluoromethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-methyl-3-(trifluoromethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-methyl-3-(trifluoromethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-methyl-3-(trifluoromethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 91 | 2-((1R,2R)-1-(2-cyanophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 92 | 2-((1S,2R)-1-(2-chloro-5-fluorophenyl)-1-(1-(2-hydroxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-hydroxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-hydroxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-hydroxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 93 | 2-((1S,2R)-1-(2-chloro-5-fluorophenyl)-1-(1-(2-methoxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-methoxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-methoxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-methoxy-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 94 | 2-((1S,2R)-1-(2-chloro-5-fluorophenyl)-1-(1-(oxetan-3-ylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(oxetan-3-ylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(oxetan-3-ylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(oxetan-3-ylmethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 95 | 2-((1S,2R)-1-(2-cyanophenyl)-1-(3,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(3,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3,5-dimethyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 96 | 2-((1S,2R)-1-(2-chlorophenyl)-1-(5-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(5-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(5-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(5-cyano-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 97 | 2-((1S,2R)-1-(2-chloro-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-methoxyethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 98 | 2-((1S,2R)-1-(2-chloro-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 99 | 2-((1S,2R)-1-(2-chloro-5-fluorophenyl)-1-(1-(2-cyano-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-cyano-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-cyano-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-cyano-2-methylpropyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 100 | 2-((1S,2R)-1-(2-chloro-5-fluorophenyl)-1-(1-(1-cyano-2-methylpropan-2-yl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(1-cyano-2-methylpropan-2-yl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(1-cyano-2-methylpropan-2-yl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(1-cyano-2-methylpropan-2-yl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 101 | 2-((1R,2R)-1-(2-cyano-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2-(trifluoromethoxy)ethyl)-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 102 | 2-((1R,2R)-1-(2-cyano-5-fluorophenyl)-1-(1-(2,2-difluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2,2-difluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2,2-difluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2,2-difluoroethyl)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 103 | 2-((1R,2R)-1-(2-cyano-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 104 | 2-((1R,2R)-1-(2-cyano-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 105 | 2-((1R,2R)-1-(2-cyanophenyl)-1-(1-isopropyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-isopropyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-isopropyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-isopropyl-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 106 | 2-((1R,2R)-1-(2-cyanophenyl)-1-(1-(oxetan-3-yl)-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(oxetan-3-yl)-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-(oxetan-3-yl)-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(oxetan-3-yl)-1H-pyrazol-4-yl)propan-2-yl)- |
| | 5-hydroxy-N-(isoxazol-4-yl)-1-methyl-6-oxo-1,6-dihydropyrimidine-4- |
| | carboxamide |
| 107 | 2-((1S,2R)-1-(2-chloro-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-methoxyethyl)-3-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 108 | 2-((1S,2R)-1-(2-chloro-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-chloro-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1-(2-methoxyethyl)-5-methyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 109 | 2-((1R,2R)-1-(2-cyano-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 110 | 2-((1R,2R)-1-(2-cyano-5-fluorophenyl)-1-(1-(2-methoxyethyl)-3,5-dimethyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(1-(2-methoxyethyl)-3,5-dimethyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(1-(2-methoxyethyl)-3,5-dimethyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(1-(2-methoxyethyl)-3,5-dimethyl-1H- |
| | pyrazol-4-yl)propan-2-yl)-5-hydroxy-N-(isoxazol-4-yl)-1-methyl-6-oxo-1,6- |
| | dihydropyrimidine-4-carboxamide |
| 111 | 2-((1R,2R)-1-(2-cyano-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| 112 | 2-((1R,2R)-1-(2-cyano-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| 113 | 2-((1R,2R)-1-(2-chloro-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(1,3-dimethyl-1H-pyrazol-5-yl)propan- |
| | 2-yl)-5-hydroxy-N-(isoxazol-4-yl)-1-methyl-6-oxo-1,6-dihydropyrimidine-4- |
| | carboxamide |
| 114 | 2-((1R,2R)-1-(2-chloro-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(1,5-dimethyl-1H-pyrazol-3-yl)propan- |
| | 2-yl)-5-hydroxy-N-(isoxazol-4-yl)-1-methyl-6-oxo-1,6-dihydropyrimidine-4- |
| | carboxamide |
| 115 | 2-((1R,2R)-1-(2-cyano-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| 116 | 2-((1R,2R)-1-(2-chlorophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1S,2S)-1-(2-chlorophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1S,2R)-1-(2-chlorophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| | 2-((1R,2S)-1-(2-chlorophenyl)-1-(5-methylpyrazin-2-yl)propan-2-yl)-5-hydroxy- |
| | N-(isoxazol-4-yl)-1-methyl-6-oxo-1,6-dihydropyrimidine-4-carboxamide |
| 117 | 2-((1R,2R)-1-(2-chloro-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(5-methylpyrazin-2-yl)propan-2-yl)-5- |
| | hydroxy-N-(isoxazol-4-yl)-1-methyl-6-oxo-1,6-dihydropyrimidine-4- |
| | carboxamide |
| 118 | 2-((1R,2R)-1-(2-cyanophenyl)-1-(5-(trifluoromethyl)pyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(5-(trifluoromethyl)pyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(5-(trifluoromethyl)pyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(5-(trifluoromethyl)pyrazin-2-yl)propan-2-yl)-5- |
| | hydroxy-N-(isoxazol-4-yl)-1-methyl-6-oxo-1,6-dihydropyrimidine-4- |
| | carboxamide |
| 119 | 2-((1R,2R)-1-(2-cyano-5-fluorophenyl)-1-(3,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(3,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(3,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(3,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| 120 | 2-((1R,2R)-1-(2-chlorophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(3,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 121 | 2-((1R,2R)-1-(2-chloro-5-fluorophenyl)-1-(3,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(3,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(3,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(3,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| 122 | 2-((1R,2R)-1-(2-cyanophenyl)-1-(3,5,6-trimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3,5,6-trimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3,5,6-trimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3,5,6-trimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 123 | 2-((1R,2R)-1-(2-cyanophenyl)-1-(3,5-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3,5-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3,5-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3,5-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 124 | 2-((1R,2R)-1-(2-cyano-5-fluorophenyl)-1-(5,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyano-5-fluorophenyl)-1-(5,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| | 2-((1S,2R)-1-(2-cyano-5-fluorophenyl)-1-(5,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| | 2-((1R,2S)-1-(2-cyano-5-fluorophenyl)-1-(5,6-dimethylpyrazin-2-yl)propan-2-yl)- |
| | 5-hydroxy-N-(isoxazol-4-yl)-1-methyl-6-oxo-1,6-dihydropyrimidine-4- |
| | carboxamide |
| 125 | 2-((1R,2R)-1-(2-chlorophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(5,6-dimethylpyrazin-2-yl)propan-2-yl)-5- |
| | hydroxy-N-(isoxazol-4-yl)-1-methyl-6-oxo-1,6-dihydropyrimidine-4- |
| | carboxamide |
| 126 | 2-((1R,2R)-1-(2-chloro-5-fluorophenyl)-1-(5,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(5,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(5,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(5,6-dimethylpyrazin-2-yl)propan-2- |
| | yl)-5-hydroxy-N-(isoxazol-4-yl)-1-methyl-6-oxo-1,6-dihydropyrimidine-4- |
| | carboxamide |
| 127 | 2-((1R,2R)-1-(2-cyanophenyl)-1-(3-fluoro-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3-fluoro-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3-fluoro-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3-fluoro-1H-pyrazol-4-yl)propan-2-yl)-5- |
| | hydroxy-N-(isoxazol-4-yl)-1-methyl-6-oxo-1,6-dihydropyrimidine-4- |
| | carboxamide |
| 128 | 2-((1R,2R)-1-(2-cyanophenyl)-1-(3-fluoro-1-isopropyl-1H-pyrazol-4-yl)propan- |
| | 2-yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3-fluoro-1-isopropyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3-fluoro-1-isopropyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3-fluoro-1-isopropyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 129 | 2-((1R,2R)-1-(2-cyano-5-fluorophenyl)-1-(3-fluoro-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyano-5-fluorophenyl)-1-(3-fluoro-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyano-5-fluorophenyl)-1-(3-fluoro-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyano-5-fluorophenyl)-1-(3-fluoro-1-methyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 130 | 2-((1S,2R)-1-(2-chlorophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(3-fluoro-1-methyl-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 131 | 2-((1S,2R)-1-(2-cyanophenyl)-1-(1-methyl-1H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-methyl-1H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-cyanophenyl)-1-(1-methyl-1H-tetrazol-5-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-methyl-1H-tetrazol-5-yl)propan-2-yl)-5- |
| | hydroxy-N-(isoxazol-4-yl)-1-methyl-6-oxo-1,6-dihydropyrimidine-4- |
| | carboxamide |
| 132 | 2-((1R,2R)-1-(2-cyanophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 133 | 2-((1R,2R)-1-(2-chlorophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(4-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 134 | 2-((1R,2R)-1-(2-chlorophenyl)-1-(4-ethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-ethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-ethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(4-ethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 135 | 2-((1R,2R)-1-(2-chlorophenyl)-1-(4-isopropyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-isopropyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-isopropyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(4-isopropyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 136 | 2-((1R,2R)-1-(2-chlorophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 137 | 2-((1R,2R)-1-(2-cyanophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(3,5-dimethyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 138 | 2-((1R,2R)-1-(2-cyanophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(5-methyl-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 139 | 2-((1R,2R)-1-(2-chlorophenyl)-1-(4-(trifluoromethyl)-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-(trifluoromethyl)-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-(trifluoromethyl)-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(4-(trifluoromethyl)-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| 140 | 2-((1R,2R)-1-(2-chlorophenyl)-1-(5-cyano-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(5-cyano-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(5-cyano-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(5-cyano-1H-pyrazol-1-yl)propan-2-yl)-5- |
| | hydroxy-N-(isoxazol-4-yl)-1-methyl-6-oxo-1,6-dihydropyrimidine-4- |
| | carboxamide |
| 141 | 2-((1R,2R)-1-(2-chlorophenyl)-1-(2H-indazol-2-yl)propan-2-yl)-5-hydroxy-N- |
| | (isoxazol-4-yl)-1-methyl-6-oxo-1,6-dihydropyrimidine-4-carboxamide |
| | 2-((1S,2S)-1-(2-chlorophenyl)-1-(2H-indazol-2-yl)propan-2-yl)-5-hydroxy-N- |
| | (isoxazol-4-yl)-1-methyl-6-oxo-1,6-dihydropyrimidine-4-carboxamide |
| | 2-((1R,2S)-1-(2-chlorophenyl)-1-(2H-indazol-2-yl)propan-2-yl)-5-hydroxy-N- |
| | (isoxazol-4-yl)-1-methyl-6-oxo-1,6-dihydropyrimidine-4-carboxamide |
| | 2-((1S,2R)-1-(2-chlorophenyl)-1-(2H-indazol-2-yl)propan-2-yl)-5-hydroxy-N- |
| | (isoxazol-4-yl)-1-methyl-6-oxo-1,6-dihydropyrimidine-4-carboxamide |
| 142 | 2-((1R,2R)-1-(2-chlorophenyl)-1-(4-methyl-1H-imidazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-methyl-1H-imidazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-methyl-1H-imidazol-1-yl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(2-chlorophenyl)-1-(4-methyl-1H-imidazol-1-yl)propan-2-yl)-5- |
| | hydroxy-N-(isoxazol-4-yl)-1-methyl-6-oxo-1,6-dihydropyrimidine-4- |
| | carboxamide |
| 143 | 2-((1R,2R)-1-(1H-benzo[d]imidazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(1H-benzo[d]imidazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(1H-benzo[d]imidazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(1H-benzo[d]imidazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| 144 | 2-((1R,2R)-1-(2-chloro-5-fluorophenyl)-1-(4-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(4-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(4-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(4-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| 145 | 2-((1R,2R)-1-(2-chloro-5-fluorophenyl)-1-(5-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(5-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(5-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(5-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| 146 | 2-((1R,2R)-1-(2-chloro-5-fluorophenyl)-1-(3-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-chloro-5-fluorophenyl)-1-(3-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-chloro-5-fluorophenyl)-1-(3-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-chloro-5-fluorophenyl)-1-(3-methyl-1H-pyrazol-1-yl)propan-2- |
| | yl)-5-hydroxy-N-(isoxazol-4-yl)-1-methyl-6-oxo-1,6-dihydropyrimidine-4- |
| | carboxamide |
| 147 | 2-((1R,2R)-1-(5-carbamoyl-1H-pyrazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(5-carbamoyl-1H-pyrazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(5-carbamoyl-1H-pyrazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| | 2-((1S,2R)-1-(5-carbamoyl-1H-pyrazol-1-yl)-1-(2-chlorophenyl)propan-2-yl)-5- |
| | hydroxy-N-(isoxazol-4-yl)-1-methyl-6-oxo-1,6-dihydropyrimidine-4- |
| | carboxamide |
| 149 | 2-((1S,2R)-1-(2-cyanophenyl)-1-(1-methyl-1H-pyrazol-4-yl)propan-2-yl)-1-ethyl- |
| | 5-hydroxy-N-(isoxazol-4-yl)-6-oxo-1,6-dihydropyrimidine-4-carboxamide |
| | 2-((1R,2S)-1-(2-cyanophenyl)-1-(1-methyl-1H-pyrazol-4-yl)propan-2-yl)-1-ethyl- |
| | 5-hydroxy-N-(isoxazol-4-yl)-6-oxo-1,6-dihydropyrimidine-4-carboxamide |
| | 2-((1S,2S)-1-(2-cyanophenyl)-1-(1-methyl-1H-pyrazol-4-yl)propan-2-yl)-1-ethyl- |
| | 5-hydroxy-N-(isoxazol-4-yl)-6-oxo-1,6-dihydropyrimidine-4-carboxamide |
| | 2-((1R,2S)-1-(2-cyanophenyl)-1-(1-methyl-1H-pyrazol-4-yl)propan-2-yl)-1-ethyl- |
| | 5-hydroxy-N-(isoxazol-4-yl)-6-oxo-1,6-dihydropyrimidine-4-carboxamide |
| 150 | 2-((1S,2R)-1-(2-cyanophenyl)-1-(1-methyl-1H-pyrazol-4-yl)propan-2-yl)-5- |
| | hydroxy-1-isopropyl-N-(isoxazol-4-yl)-6-oxo-1,6-dihydropyrimidine-4- |
| | carboxamide |
| | 2-((1R,2S)-1-(2-cyanophenyl)-1-(1-methyl-1H-pyrazol-4-yl)propan-2-yl)-5- |
| | hydroxy-1-isopropyl-N-(isoxazol-4-yl)-6-oxo-1,6-dihydropyrimidine-4- |
| | carboxamide |
| | 2-((1S,2S)-1-(2-cyanophenyl)-1-(1-methyl-1H-pyrazol-4-yl)propan-2-yl)-5- |
| | hydroxy-1-isopropyl-N-(isoxazol-4-yl)-6-oxo-1,6-dihydropyrimidine-4- |
| | carboxamide |
| | 2-((1R,2R)-1-(2-cyanophenyl)-1-(1-methyl-1H-pyrazol-4-yl)propan-2-yl)-5- |
| | hydroxy-1-isopropyl-N-(isoxazol-4-yl)-6-oxo-1,6-dihydropyrimidine-4- |
| | carboxamide |
| 151 | 2-((1R,2R)-1-(2-cyanophenyl)-1-(1-(trifluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2S)-1-(2-cyanophenyl)-1-(1-(trifluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1S,2R)-1-(2-cyanophenyl)-1-(1-(trifluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| | 2-((1R,2S)-1-(2-cyanophenyl)-1-(1-(trifluoromethyl)-1H-pyrazol-4-yl)propan-2- |
| | yl)-5-hydroxy-N-(isoxazol-4-yl)-1-methyl-6-oxo-1,6-dihydropyrimidine-4- |
| | carboxamide |
| 152 | 2-((1S,2R)-1-(2-chlorophenyl)-1-(4-methyl-4H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(4-methyl-4H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(4-methyl-4H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(4-methyl-4H-1,2,4-triazol-3-yl)propan-2-yl)-5- |
| | hydroxy-N-(isoxazol-4-yl)-1-methyl-6-oxo-1,6-dihydropyrimidine-4- |
| | carboxamide |
| 153 | 2-((1R,2R)-1-(2-cyanophenyl)-1-(1-methyl-3-(N-methylacetamido)-1H-pyrazol- |
| | 4-yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-cyanophenyl)-1-(1-methyl-3-(N-methylacetamido)-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2S)-1-(2-cyanophenyl)-1-(1-methyl-3-(N-methylacetamido)-1H-pyrazol- |
| | 4-yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-cyanophenyl)-1-(1-methyl-3-(N-methylacetamido)-1H-pyrazol- |
| | 4-yl)propan-2-yl)-5-hydroxy-N-(isoxazol-4-yl)-1-methyl-6-oxo-1,6- |
| | dihydropyrimidine-4-carboxamide |
| 154 | 2-((1R,2S)-1-(2-chloro-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2R)-1-(2-chloro-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1R,2R)-1-(2-chloro-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| | 2-((1S,2S)-1-(2-chloro-5-fluorophenyl)-1-(1,3,5-trimethyl-1H-pyrazol-4- |
| | yl)propan-2-yl)-5-hydroxy-N-(isoxazol-4-yl)-1-methyl-6-oxo-1,6- |
| | dihydropyrimidine-4-carboxamide |
| 155 | 2-((1S,2R)-1-(2-cyanopyridin-3-yl)-1-phenylpropan-2-yl)-5-hydroxy-N- |
| | (isoxazol-4-yl)-1-methyl-6-oxo-1,6-dihydropyrimidine-4-carboxamide |
| | 2-((1R,2S)-1-(2-cyanopyridin-3-yl)-1-phenylpropan-2-yl)-5-hydroxy-N- |
| | (isoxazol-4-yl)-1-methyl-6-oxo-1,6-dihydropyrimidine-4-carboxamide |
| | 2-((1R,2R)-1-(2-cyanopyridin-3-yl)-1-phenylpropan-2-yl)-5-hydroxy-N- |
| | (isoxazol-4-yl)-1-methyl-6-oxo-1,6-dihydropyrimidine-4-carboxamide |
| | 2-((1S,2S)-1-(2-cyanopyridin-3-yl)-1-phenylpropan-2-yl)-5-hydroxy-N-(isoxazol- |
| | 4-yl)-1-methyl-6-oxo-1,6-dihydropyrimidine-4-carboxamide |
| 156 | 2-((1S,2R)-1-(2-chlorophenyl)-1-(5-methyl-1,3,4-oxadiazol-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2S)-1-(2-chlorophenyl)-1-(5-methyl-1,3,4-oxadiazol-2-yl)propan-2-yl)-5- |
| | hydroxy-N-(isoxazol-4-yl)-1-methyl-6-oxo-1,6-dihydropyrimidine-4- |
| | carboxamide |
| | 2-((1R,2R)-1-(2-chlorophenyl)-1-(5-methyl-1,3,4-oxadiazol-2-yl)propan-2-yl)-5- |
| | hydroxy-N-(isoxazol-4-yl)-1-methyl-6-oxo-1,6-dihydropyrimidine-4- |
| | carboxamide |
| | 2-((1S,2S)-1-(2-chlorophenyl)-1-(5-methyl-1,3,4-oxadiazol-2-yl)propan-2-yl)-5- |
| | hydroxy-N-(isoxazol-4-yl)-1-methyl-6-oxo-1,6-dihydropyrimidine-4- |
| | carboxamide |

hTREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length human TREX1 (expressed in E. coli and purified in house) in reaction buffer (50 mM Tris, 150 mM NaCl, 2 mM DTT, 0.1 mg/mL BSA, 0.01% (w/v) Tween-20, 5 mM MgCl2, pH 7.4) was added to a 384-well Black ProxiPlate Plus (PerkinElmer) which already contained compound (150 nL) at varying concentrations as a 10 point dose-response in DMSO. The plate was incubated at 25° C. for 4 hours. Reactions were initiated by adding 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 4 pM TREX1, 60 nM dsDNA substrate in reaction buffer with 1.0% DMSO (v/v). After 18 hours at 25° C., reactions were quenched by the addition of 2 μL of 500 mM EDTA. Final concentrations in the quenched reaction were 3.5 pM TREX1, 53 nM DNA and 59 mM EDTA in a volume of 17 μL. After a 5-minute incubation at room temperature, plates were read in an EnVision plate reader (PerkinElmer) measuring fluorescence at 615 nm following excitation w/570 nm light. IC50 values were calculated by comparing the measured fluorescence at 615 nm relative to control wells pre-quenched w/EDTA (100% inhibition) and no inhibitor (0% inhibition) controls as using non-linear least square four parameter fits and either Genedata or GraphPad Prism (GraphPad Software, Inc.).

mTREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length mouse TREX1 (expressed in E. coli and purified in house) in reaction buffer (50 mM Tris, 150 mM NaCl, 2 mM DTT, 0.1 mg/mL BSA, 0.01% (w/v) Tween-20, 5 mM MgCl2, pH 7.4) was added to a 384-well Black ProxiPlate Plus (PerkinElmer) which already contained compound (150 nL) at varying concentrations as a 10 point dose-response in DMSO. The plate was incubated at 25° C. for 4 hours. Reactions were initiated by adding 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 6 pM TREX1, 60 nM dsDNA substrate in reaction buffer with 1.0% DMSO (v/v). After 18 hours at 25° C., reactions were quenched by the addition of 2 μL of 500 mM EDTA. Final concentrations in the quenched reaction were 5.3 pM TREX1, 53 nM DNA and 59 mM EDTA in a volume of 17 μL. After a 5-minute incubation at room temperature, plates were read in an EnVision plate reader (PerkinElmer) measuring fluorescence at 615 nm following excitation w/570 nm light. IC50 values were calculated by comparing the measured fluorescence at 615 nm relative to control wells pre-quenched w/EDTA (100% inhibition) and no inhibitor (0% inhibition) controls as using non-linear least square four parameter fits and either Genedata or GraphPad Prism (GraphPad Software, Inc.).

hTREX2 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged human TREX2 (residues M44-A279, expressed in E. coli and purified in house) in reaction buffer (50 mM Tris, 150 mM NaCl, 2 mM DTT, 0.1 mg/mL BSA, 0.01% (w/v) Tween-20, 5 mM MgCl2, pH 7.4) was added to a 384-well Black ProxiPlate Plus (PerkinElmer) which already contained compound (150 nL) at varying concentrations as a 10 point dose-response in DMSO. The plate was incubated at 25° C. for 4 hours. Reactions were initiated by adding 7.5 μL of dsDNA substrate (Strand A: 5′ TEX615/GCT AGG CAG 3′; Strand B: 5′ CTG CCT AGC/IAbRQSp (IDT)) in reaction buffer. Final concentrations were 50 pM TREX2, 60 nM dsDNA substrate in reaction buffer with 1.0% DMSO (v/v). After 18 hours at 25° C., reactions were quenched by the addition of 2 μL of 500 mM EDTA. Final concentrations in the quenched reaction mixture were 44 pM TREX2, 53 nM DNA and 59 mM EDTA in a volume of 17 μL. After a 5-minute incubation at room temperature, plates were read in an EnVision plate reader (PerkinElmer) measuring fluorescence at 615 nm following excitation w/570 nm light. IC50 values were calculated by comparing the measured fluorescence at 615 nm relative to control wells pre-quenched w/stop buffer (100% inhibition) and no inhibitor (0% inhibition) controls as using non-linear least square four parameter fits and either Genedata or GraphPad Prism (GraphPad Software, Inc.).

TABLE 7
hTREX1 and mTREX1 biochemical IC50 data, and
biochemical selectivity for hTREX1 vs hTREX2.

| | | | hTREX1 |
|---|---|---|---|
| | hTREX1 | mTREX1 | vs |
| | IC50 | IC50 | selectivity |
| Example 1 D1E1 | B | B | A |
| Example 1 D1E2 | C | C | A |
| Example 1 D2E1 | C | C | |
| Example 1 D2E2 | C | C | B |
| Example 2 D1E1 | A | B | C |
| Example 2 D1E2 | C | C | A |
| Example 2 D2E1 | C | C | |
| Example 2 D2E2 | C | C | A |
| Example 3 D1E1 | A | A | C |
| Example 3 D1E2 | C | C | |
| Example 3 D2E1 | C | C | |
| Example 3 D2E2 | B | C | |
| Example 4 D1E1 | A | B | |
| Example 4 D1E2 | C | C | |
| Example 4 D2E1 | C | C | |
| Example 4 D2E2 | B | C | B |
| Example 5 D1E1 | C | C | |
| Example 5 D1E2 | A | A | B |
| Example 5 D2E1 | C | C | B |
| Example 5 D2E2 | B | C | |
| Example 6 D1E1 | A | A | C |
| Example 6 D1E2 | B | C | B |
| Example 6 D2E1 | C | C | |
| Example 6 D2E2 | B | C | |
| Example 7 D1E1 | B | C | |
| Example 7 D1E2 | C | C | |
| Example 7 D2E1 | C | C | |
| Example 7 D2E2 | C | C | B |
| Example 8 D1E1 | C | C | |
| Example 8 D1E2 | A | B | |
| Example 8 D2E1 | C | C | |
| Example 8 D2E2 | B | C | |
| Example 9 D1E1 | A | B | C |
| Example 9 D1E2 | C | C | |
| Example 9 D2E1 | B | C | C |
| Example 9 D2E2 | C | C | |
| Example 10 D1E1 | A | A | C |
| Example 10 D1E2 | B | C | |
| Example 10 D2E1 | A | B | C |
| Example 10 D2E2 | C | C | |
| Example 11 D1E1 | A | A | B |
| Example 11 D1E2 | C | C | B |
| Example 11 D2E1 | C | C | |
| Example 11 D2E2 | C | C | |
| Example 12 D1E1 | A | B | C |
| Example 12 D1E2 | C | C | B |
| Example 12 D2E1 | B | C | C |
| Example 12 D2E2 | C | C | |
| Example 13 D1E1 | A | A | C |
| Example 13 D1E2 | C | C | B |
| Example 13 D2E1 | B | C | B |
| Example 13 D2E2 | C | C | |
| Example 14 D1E1 | C | C | |
| Example 14 D1E2 | A | B | C |
| Example 14 D2E1 | C | C | A |
| Example 14 D2E2 | C | C | |
| Example 15 D1E1 | A | B | C |
| Example 15 D1E2 | C | C | |
| Example 15 D2E1 | C | C | B |
| Example 15 D2E2 | C | C | |
| Example 16 D1E1 | A | A | C |
| Example 16 D1E2 | C | C | |
| Example 16 D2E1 | C | C | |
| Example 16 D2E2 | C | C | |
| Example 17 D1E1 | C | C | |
| Example 17 D1E2 | A | A | |
| Example 17 D2E1 | C | C | B |
| Example 17 D2E2 | C | C | |
| Example 18 D1E1 | C | C | |
| Example 18 D1E2 | A | B | |
| Example 18 D2E1 | C | C | |
| Example 18 D2E2 | C | C | |
| Example 19 D1E1 | C | C | |
| Example 19 D1E2 | A | B | |
| Example 19 D2E1 | C | C | |
| Example 19 D2E2 | C | C | |
| Example 20 D1E1 | A | B | C |
| Example 20 D1E2 | C | C | |
| Example 20 D2E1 | C | C | B |
| Example 20 D2E2 | C | C | |
| Example 21 D1E1 | A | B | C |
| Example 21 D1E2 | C | C | |
| Example 21 D2E1 | C | C | B |
| Example 21 D2E2 | C | C | |
| Example 22 D1E1 | A | B | |
| Example 22 D1E2 | C | C | |
| Example 22 D2E1 | C | C | B |
| Example 22 D2E2 | C | C | |
| Example 23 D1E1 | C | C | |
| Example 23 D1E2 | A | B | C |
| Example 23 D2E1 | C | C | |
| Example 23 D2E2 | C | C | |
| Example 24 D1E1 | A | A | C |
| Example 24 D1E2 | C | C | |
| Example 24 D2E1 | C | C | |
| Example 24 D2E2 | C | C | |
| Example 25 D1E1 | A | B | C |
| Example 25 D1E2 | C | C | |
| Example 25 D2E1 | C | C | |
| Example 25 D2E2 | C | C | |
| Example 26 D1E1 | C | C | |
| Example 26 D1E2 | A | B | |
| Example 26 D2E1 | C | C | B |
| Example 26 D2E2 | C | C | |
| Example 27 D1E1 | A | A | C |
| Example 27 D1E2 | C | C | C |
| Example 27 D2E1 | C | C | |
| Example 27 D2E2 | C | C | |
| Example 28 D1E1 | A | B | |
| Example 28 D1E2 | C | C | |
| Example 28 D2E1 | C | C | |
| Example 28 D2E2 | C | C | |
| Example 29 D1E1 | A | B | C |
| Example 29 D1E2 | C | C | |
| Example 29 D2E1 | C | C | |
| Example 29 D2E2 | C | C | |
| Example 30 D1E1 | A | A | C |
| Example 30 D1E2 | C | C | |
| Example 30 D2E1 | C | C | |
| Example 30 D2E2 | C | C | |
| Example 31 D1E1 | A | B | |
| Example 31 D1E2 | C | C | |
| Example 31 D2E1 | C | C | |
| Example 31 D2E2 | C | C | |
| Example 32 D1E1 | A | B | |
| Example 32 D1E2 | B | C | |
| Example 32 D2E1 | C | C | C |
| Example 32 D2E2 | C | C | |
| Example 33 D1E1 | B | B | C |
| Example 33 D1E2 | C | C | |
| Example 33 D2E1 | C | C | |
| Example 33 D2E2 | C | C | |
| Example 34 D1E1 | A | B | |
| Example 34 D1E2 | C | C | |
| Example 34 D2E1 | C | C | C |
| Example 34 D2E2 | C | C | |
| Example 35 D1E1 | A | B | C |
| Example 35 D1E2 | C | C | |
| Example 35 D2E1 | B | C | |
| Example 35 D2E2 | C | C | |
| Example 36 D1E1 | A | B | |
| Example 36 D1E2 | C | C | |
| Example 36 D2E1 | C | C | B |
| Example 36 D2E2 | C | C | |
| Example 37 D1E1 | A | B | |
| Example 37 D1E2 | C | C | |
| Example 37 D2E1 | C | C | |
| Example 37 D2E2 | C | | |
| Example 38 D1E1 | A | A | |
| Example 38 D1E2 | C | C | |
| Example 38 D2E1 | C | C | |
| Example 38 D2E2 | B | C | |
| Example 39 D1E1 | C | C | |
| Example 39 D1E2 | C | C | |
| Example 39 D2E1 | C | C | |
| Example 39 D2E2 | B | C | C |
| Example 40 D1E1 | C | C | |
| Example 40 D1E2 | A | B | B |
| Example 40 D2E1 | C | C | |
| Example 40 D2E2 | C | C | B |
| Example 41 D1E1 | C | C | |
| Example 41 D1E2 | A | B | C |
| Example 41 D2E1 | B | C | |
| Example 41 D2E2 | C | C | |
| Example 42 D1E1 | C | C | |
| Example 42 D1E2 | A | B | C |
| Example 42 D2E1 | B | C | B |
| Example 42 D2E2 | C | C | |
| Example 148 D1 | B | C | C |
| Example 148 D2 | C | C | |
| Example 44 D1E1 | C | C | A |
| Example 44 D1E2 | B | B | B |
| Example 44 D2E1 | C | C | |
| Example 44 D2E2 | C | C | A |
| Example 45 D1E1 | B | C | B |
| Example 45 D1E2 | C | C | A |
| Example 47 D1E1 | A | | |
| Example 47 D1E2 | C | | |
| Example 49 D1E1 | B | B | |
| Example 49 D1E2 | C | C | |
| Example 50 D1E1 | B | C | |
| Example 50 D1E2 | C | C | |
| Example 50 D2E1 | C | C | C |
| Example 50 D2E2 | C | C | |
| Example 51 D1E1 | C | C | |
| Example 51 D1E2 | B | C | |
| Example 51 D2E1 | C | C | |
| Example 51 D2E2 | C | C | |
| Example 52 D1E1 | B | C | |
| Example 52 D1E2 | C | C | |
| Example 52 D2E1 | C | C | |
| Example 52 D2E2 | C | C | |
| Example 53 D1E1 | C | C | |
| Example 53 D1E2 | A | B | C |
| Example 53 D2E1 | C | C | |
| Example 53 D2E2 | C | C | |
| Example 54 D1E1 | B | B | |
| Example 54 D1E2 | C | C | |
| Example 55 D1E1 | A | A | C |
| Example 55 D1E2 | C | C | |
| Example 55 D2E1 | C | | |
| Example 55 D2E2 | B | C | C |
| Example 56 D1E1 | A | A | C |
| Example 56 D1E2 | C | C | |
| Example 56 D2E1 | B | C | |
| Example 56 D2E2 | C | C | |
| Example 57 D1E1 | B | C | C |
| Example 57 D1E2 | A | B | C |
| Example 57 D2E1 | B | C | C |
| Example 57 D2E2 | C | C | |
| Example 59 D1E1 | C | C | |
| Example 59 D1E2 | A | A | C |
| Example 60 D1E1 | A | B | B |
| Example 60 D1E2 | B | C | A |
| Example 60 D2E1 | C | C | A |
| Example 60 D2E2 | C | C | A |
| Example 61 D1E1 | A | B | |
| Example 61 D1E2 | C | C | |
| Example 61 D2E1 | C | C | |
| Example 61 D2E2 | C | C | |
| Example 62 D1E1 | C | C | |
| Example 62 D1E2 | C | C | |
| Example 62 D2E1 | C | C | |
| Example 62 D2E2 | A | B | |
| Example 63 D1E1 | A | B | C |
| Example 63 D1E2 | B | C | C |
| Example 63 D2E1 | C | C | |
| Example 63 D2E2 | C | C | |
| Example 64 D1E1 | A | C | |
| Example 64 D1E2 | C | C | |
| Example 64 D2E1 | A | B | C |
| Example 64 D2E2 | C | C | |
| Example 65 D1E1 | A | A | |
| Example 65 D1E2 | C | C | |
| Example 65 D2E1 | B | C | C |
| Example 65 D2E2 | A | C | |
| Example 66 D1E1 | A | A | B |
| Example 66 D1E2 | B | C | |
| Example 66 D2E1 | B | C | |
| Example 66 D2E2 | C | C | |
| Example 67 D1E1 | C | | |
| Example 67 D1E2 | A | | |
| Example 67 D2E1 | C | | |
| Example 67 D2E2 | C | | |
| Example 68 D1E1 | A | A | |
| Example 68 D1E2 | C | C | |
| Example 68 D2E1 | A | C | B |
| Example 68 D2E2 | C | C | |
| Example 69 D1E1 | B | B | B |
| Example 69 D1E2 | C | C | |
| Example 69 D2E1 | C | C | |
| Example 69 D2E2 | B | C | B |
| Example 70 D1E1 | A | A | C |
| Example 70 D1E2 | C | C | |
| Example 70 D2E1 | B | C | |
| Example 70 D2E2 | C | C | |
| Example 71 D1E1 | A | A | C |
| Example 71 D1E2 | C | C | |
| Example 71 D2E1 | C | C | |
| Example 71 D2E2 | C | C | |
| Example 72 D1E1 | A | B | |
| Example 72 D1E2 | C | C | |
| Example 72 D2E1 | C | C | |
| Example 72 D2E2 | C | C | |
| Example 73 D1E1 | A | B | C |
| Example 73 D1E2 | C | C | |
| Example 73 D2E1 | C | C | |
| Example 73 D2E2 | C | C | |
| Example 74 D1E1 | A | | |
| Example 74 D1E2 | C | | |
| Example 74 D2E1 | C | | |
| Example 74 D2E2 | C | | |
| Example 75 D1E1 | A | B | |
| Example 75 D1E2 | C | C | |
| Example 75 D2E1 | C | C | |
| Example 75 D2E2 | C | C | |
| Example 76 D1E1 | A | | |
| Example 77 D1E1 | A | B | C |
| Example 77 D1E2 | C | C | A |
| Example 77 D2E1 | C | C | A |
| Example 77 D2E2 | C | C | A |
| Example 78 D1E1 | A | B | |
| Example 78 D1E2 | C | C | A |
| Example 78 D2E1 | B | C | |
| Example 78 D2E2 | C | C | |
| Example 79 D1E1 | C | C | A |
| Example 79 D1E2 | B | C | B |
| Example 79 D2E1 | B | C | A |
| Example 79 D2E2 | C | C | A |
| Example 80 D1E1 | A | B | C |
| Example 80 D1E2 | C | C | C |
| Example 80 D2E1 | C | C | |
| Example 80 D2E2 | B | C | B |
| Example 81 D1E1 | C | C | A |
| Example 81 D1E2 | A | B | C |
| Example 81 D2E1 | B | C | B |
| Example 81 D2E2 | C | C | B |
| Example 82 D1E1 | A | B | C |
| Example 82 D1E2 | C | C | B |
| Example 82 D2E1 | B | C | C |
| Example 82 D2E2 | C | C | B |
| Example 83 D1E1 | A | C | C |
| Example 83 D1E2 | C | C | A |
| Example 83 D2E1 | B | C | B |
| Example 83 D2E2 | C | C | A |
| Example 84 D1E1 | A | B | B |
| Example 84 D1E2 | C | C | |
| Example 85 D1E1 | A | A | B |
| Example 85 D1E2 | B | C | A |
| Example 85 D2E1 | C | C | A |
| Example 85 D2E2 | C | C | A |
| Example 86 D1E1 | A | B | B |
| Example 86 D1E2 | B | C | A |
| Example 86 D2E1 | C | C | A |
| Example 86 D2E2 | C | C | A |
| Example 87 D1E1 | C | C | |
| Example 87 D2E1 | C | C | |
| Example 88 D1E1 | A | B | A |
| Example 88 D1E2 | B | C | A |
| Example 88 D2E1 | C | C | A |
| Example 88 D2E2 | C | C | A |
| Example 89 D1E1 | C | C | A |
| Example 89 D1E2 | A | B | C |
| Example 90 D1E1 | A | B | C |
| Example 90 D1E2 | C | C | A |
| Example 90 D2E1 | C | C | B |
| Example 90 D2E2 | B | C | B |
| Example 91 D1E1 | A | A | B |
| Example 91 D1E2 | C | C | A |
| Example 92 D1E1 | B | B | A |
| Example 92 D1E2 | B | C | A |
| Example 93 D1E1 | B | B | B |
| Example 93 D1E2 | B | C | A |
| Example 94 D1E1 | B | B | B |
| Example 94 D1E2 | C | C | A |
| Example 95 D1E1 | A | B | C |
| Example 95 D1E2 | C | C | A |
| Example 95 D2E1 | C | C | A |
| Example 95 D2E2 | C | C | A |
| Example 96 D1E1 | A | B | A |
| Example 96 D1E2 | C | C | A |
| Example 96 D2E1 | C | C | A |
| Example 96 D2E2 | A | B | C |
| Example 97 D1E1 | B | B | C |
| Example 97 D1E2 | B | C | A |
| Example 98 D1E1 | B | B | B |
| Example 98 D1E2 | C | B | A |
| Example 99 D1E1 | B | C | A |
| Example 99 D1E2 | B | B | B |
| Example 100 D1E1 | B | B | A |
| Example 100 D1E2 | B | C | A |
| Example 101 D1E1 | C | C | A |
| Example 101 D1E2 | A | B | C |
| Example 102 D1E1 | C | C | A |
| Example 102 D1E2 | A | B | B |
| Example 103 D1E1 | C | C | A |
| Example 103 D1E2 | A | A | C |
| Example 103 D2E1 | B | C | A |
| Example 103 D2E2 | C | C | A |
| Example 104 D1E1 | B | B | B |
| Example 104 D1E2 | C | C | A |
| Example 104 D2E1 | C | C | A |
| Example 104 D2E2 | B | C | B |
| Example 105 D1E1 | C | C | A |
| Example 105 D1E2 | A | B | B |
| Example 105 D2E1 | B | C | A |
| Example 105 D2E2 | C | C | A |
| Example 106 D1E1 | A | B | B |
| Example 106 D1E2 | C | C | A |
| Example 106 D2E1 | C | C | B |
| Example 106 D2E2 | C | C | |
| Example 107 D1E1 | A | B | C |
| Example 107 D1E2 | C | C | A |
| Example 107 D2E1 | C | C | A |
| Example 107 D2E2 | C | C | |
| Example 108 D1E1 | B | C | A |
| Example 108 D1E2 | C | C | A |
| Example 108 D2E1 | B | C | A |
| Example 108 D2E2 | C | C | A |
| Example 109 D1E1 | A | B | B |
| Example 109 D1E2 | C | C | A |
| Example 110 D1E1 | A | B | C |
| Example 110 D1E2 | C | C | A |
| Example 111 D1E1 | C | C | |
| Example 111 D1E2 | A | B | B |
| Example 111 D2E1 | A | C | C |
| Example 111 D2E2 | C | C | A |
| Example 112 D1E1 | A | B | C |
| Example 112 D1E2 | C | C | A |
| Example 112 D2E1 | B | C | A |
| Example 112 D2E2 | C | C | A |
| Example 113 D1E1 | C | C | C |
| Example 113 D1E2 | C | C | A |
| Example 113 D2E1 | C | C | A |
| Example 113 D2E2 | C | C | |
| Example 114 D1E1 | B | C | B |
| Example 114 D1E2 | C | C | A |
| Example 114 D2E1 | B | C | B |
| Example 114 D2E2 | C | C | |
| Example 115 D1E1 | C | C | A |
| Example 115 D1E2 | A | B | B |
| Example 115 D2E1 | C | C | |
| Example 115 D2E2 | B | C | C |
| Example 116 D1E1 | C | C | A |
| Example 116 D1E2 | A | B | B |
| Example 116 D2E1 | B | C | A |
| Example 116 D2E2 | C | C | |
| Example 117 D1E1 | C | C | A |
| Example 117 D1E2 | A | B | A |
| Example 117 D2E1 | B | C | A |
| Example 117 D2E2 | C | C | A |
| Example 118 D1E1 | B | B | B |
| Example 118 D1E2 | C | C | A |
| Example 118 D2E1 | B | C | B |
| Example 118 D2E2 | C | C | A |
| Example 119 DIE1 | A | B | C |
| Example 119 D1E2 | C | C | B |
| Example 119 D2E1 | B | C | C |
| Example 119 D2E2 | C | C | A |
| Example 120 D1E1 | A | B | C |
| Example 120 D1E2 | C | C | A |
| Example 120 D2E1 | C | C | |
| Example 120 D2E2 | C | C | C |
| Example 121 D1E1 | A | B | C |
| Example 121 D1E2 | C | C | A |
| Example 121 D2E1 | C | C | B |
| Example 121 D2E2 | C | C | |
| Example 122 D1E1 | A | A | C |
| Example 122 D1E2 | C | C | B |
| Example 122 D2E1 | B | C | B |
| Example 122 D2E2 | C | C | |
| Example 123 D1E1 | B | B | C |
| Example 123 D1E2 | C | C | A |
| Example 123 D2E1 | B | C | C |
| Example 123 D2E2 | C | C | A |
| Example 124 D1E1 | C | C | A |
| Example 124 D1E2 | A | B | B |
| Example 124 D2E1 | C | C | A |
| Example 124 D2E2 | C | C | A |
| Example 125 D1E1 | C | C | A |
| Example 125 D1E2 | A | B | B |
| Example 125 D2E1 | B | C | A |
| Example 125 D2E2 | C | C | |
| Example 126 D1E1 | B | C | A |
| Example 126 D1E2 | A | B | C |
| Example 126 D2E1 | B | C | A |
| Example 126 D2E2 | C | C | |
| Example 127 D1E1 | A | A | B |
| Example 127 D1E2 | B | C | B |
| Example 128 D1E1 | A | A | B |
| Example 128 D1E2 | C | C | A |
| Example 129 D1E1 | C | C | A |
| Example 129 D1E2 | A | A | C |
| Example 130 D1E1 | A | A | B |
| Example 130 D1E2 | C | C | A |
| Example 131 D1E1 | C | C | A |
| Example 131 D1E2 | C | C | |
| Example 131 D2E1 | C | C | C |
| Example 131 D2E2 | C | C | |
| Example 132 D1E1 | C | C | A |
| Example 132 D1E2 | A | B | C |
| Example 133 D1E1 | C | C | A |
| Example 133 D1E2 | B | B | A |
| Example 134 D1E1 | B | B | A |
| Example 134 D1E2 | C | C | A |
| Example 135 D1E1 | B | B | B |
| Example 135 D1E2 | C | C | A |
| Example 136 D1E1 | C | C | A |
| Example 136 D1E2 | A | C | B |
| Example 137 D1E1 | A | B | B |
| Example 137 D1E2 | C | C | A |
| Example 138 D1E1 | A | B | B |
| Example 138 D1E2 | C | C | A |
| Example 139 D1E1 | B | B | A |
| Example 139 D1E2 | C | C | A |
| Example 140 D1E1 | A | B | B |
| Example 140 D1E2 | C | C | A |
| Example 141 D1E1 | B | B | A |
| Example 141 D1E2 | C | C | A |
| Example 142 D1E1 | C | C | A |
| Example 142 D1E2 | A | B | A |
| Example 143 D1E1 | A | B | B |
| Example 143 D1E2 | C | C | A |
| Example 144 D1E1 | C | C | A |
| Example 144 D1E2 | B | C | A |
| Example 145 D1E1 | B | C | B |
| Example 145 D1E2 | C | C | A |
| Example 146 D1E1 | B | C | C |
| Example 146 D1E2 | C | C | A |
| Example 147 D1E1 | A | C | C |
| Example 147 D1E2 | C | C | A |
| Example 149 D1E1 | C | C | A |
| Example 149 D1E2 | A | B | C |
| Example 149 D2E1 | C | C | A |
| Example 149 D2E2 | C | C | B |
| Example 150 D1E1 | C | C | A |
| Example 150 D1E2 | A | B | C |
| Example 150 D2E1 | C | C | A |
| Example 150 D2E2 | C | C | |
| Example 151 D1E1 | A | A | B |
| Example 151 D1E2 | C | C | A |
| Example 151 D2E1 | B | C | B |
| Example 151 D2E2 | C | C | |
| Example 152 D1E1 | C | C | B |
| Example 152 D1E2 | C | C | A |
| Example 152 D2E1 | C | C | B |
| Example 152 D2E2 | C | C | A |
| Example 153 D1E1 | C | C | |
| Example 153 D1E2 | C | C | A |
| Example 154 D1E2 | C | C | A |
| Example 154 D1E1 | A | B | B |
| Example 155 D1E1 | C | C | B |
| Example 155 D1E2 | B | B | C |
| Example 155 D2E1 | A | C | C |
| Example 155 D2E2 | C | C | A |
| Example 156 D1E1 | C | C | A |
| Example 156 D1E2 | C | C | A |
| Example 156 D2E1 | C | C | A |
| Example 156 D2E2 | B | C | B |

hTREX1 IC₅₀: A = <0.001 μM; B = 0.001 to 0.01 μM; C = >0.01 μM.
mTREX1 IC50: A = <0.001 μM; B = 0.001 to 0.01 μM; C= >0.01 μM.
hTREX1 vs hTREX2 selectivity: A = <25 fold selective for hTREX1, B = 25 to 50-fold selective for hTREX1, C = >50 fold selective for hTREX1

hTREX1 HCT116 Cell Assay

HCT116 dual cells (Invivogen, San Diego, CA, USA) are derived from the human HCT116 colorectal carcinoma cell line. Cells have been selected for the stable integration of SEAP and Luciferase reporter genes, which expression is under the control of 5 tandem response elements for NF-KB/AP1 and STAT1/STAT2, respectively. The cell line was used to monitor Type I interferon induction and subsequent signaling by measuring the activity of the Lucia luciferase secreted in the culture medium.

HCT116 cells were plated in 96-well plate(s) at 40,000 cells/well in 100 uL DMEM supplemented with 10% FBS and 25 mM Hepes (pH 7.2-7.5). After overnight settling, cells were treated with TREX1i for 4 h (maximum DMSO fraction was 0.1%) before 1 ug/mL pBR322/BstNI restriction digest (New England Biolabs, Ipswich, MA, USA) was transfected with Lipofectamine LTX (ThermoFisher, Grand Island, NY, USA), according to product manual recommendations. Briefly, Lipofectamine LTX (0.35 uL/well) was diluted in OptiMEM (5 uL/well). pBR322/BstNI (100 ng/well) was diluted in OptiMEM (5 uL/well) before Plus reagent (0.1 uL/100 ng DNA) was added. After 5 min incubation at room temperature, the DNA mixture was mixed dropwise with the diluted Lipofectamine LTX. After an additional 10 min incubation, the transfection mix (10 uL/well) was added to the cells. Cells were maintained at 37 C for 48 h before monitoring the Lucia Luciferase activity from the cell culture medium.

TABLE 8
hTREX1 HCT116 Cell Assay EC50 data.
EC₅₀: A = <0.01 μM; B = 0.01 to 0.1 μM; C = >0.1 μM.

| | hTREX1 | | hTREX1 | | hTREX1 |
|---|---|---|---|---|---|
| | HCT116 | | HCT116 | | HCT116 |
| | IC50 | | IC50 | | IC50 |
| Example 1 | B | Example 61 | A | Example 108 | B |
| D1E1 | | D1E1 | | D2E1 | |
| Example 2 | A | Example 62 | A | Example 109 | B |
| D1E1 | | D2E2 | | D1E1 | |
| Example 2 | C | Example 63 | A | Example 110 | C |
| D1E2 | | D1E1 | | D1E1 | |
| Example 2 | C | Example 65 | A | Example 111 | B |
| D2E2 | | D1E1 | | D1E2 | |
| Example 3 | A | Example 65 | A | Example 111 | C |
| D1E1 | | D2E2 | | D2E1 | |
| Example 3 | C | Example 68 | A | Example 112 | B |
| D2E2 | | D1E1 | | D1E1 | |
| Example 4 | B | Example 68 | A | Example 112 | C |
| D1E1 | | D2E1 | | D2E1 | |
| Example 4 | C | Example 69 | B | Example 113 | B |
| D2E2 | | D1E1 | | D1E1 | |
| Example 5 | A | Example 69 | B | Example 114 | C |
| D1E2 | | D2E2 | | D1E1 | |
| Example 5 | C | Example 70 | A | Example 114 | C |
| D2E2 | | D1E1 | | D2E1 | |
| Example 6 | A | Example 71 | A | Example 115 | A |
| D1E1 | | D1E1 | | D1E2 | |
| Example 6 | C | Example 75 | C | Example 115 | C |
| D1E2 | | D1E1 | | D2E2 | |
| Example 6 | C | Example 77 | B | Example 116 | C |
| D2E2 | | D1E1 | | D1E1 | |
| Example 7 | C | Example 77 | C | Example 116 | B |
| D1E1 | | D1E2 | | D1E2 | |
| Example 8 | B | Example 77 | C | Example 116 | C |
| D1E2 | | D2E1 | | D2E1 | |
| Example 8 | C | Example 78 | A | Example 117 | A |
| D2E2 | | D1E1 | | D1E2 | |
| Example 9 | B | Example 78 | B | Example 117 | C |
| D1E1 | | D2E1 | | D2E1 | |
| Example 9 | C | Example 79 | C | Example 118 | B |
| D2E1 | | D1E2 | | D1E1 | |
| Example 10 | A | Example 79 | C | Example 118 | C |
| D1E1 | | D2E1 | | D2E1 | |
| Example 10 | B | Example 80 | C | Example 119 | A |
| D1E2 | | D1E1 | | D1E1 | |
| Example 10 | A | Example 80 | C | Example 119 | C |
| D2E1 | | D2E2 | | D1E2 | |
| Example 11 | C | Example 81 | A | Example 119 | C |
| D1E1 | | D1E2 | | D2E1 | |
| Example 12 | B | Example 81 | C | Example 119 | C |
| D1E1 | | D2E1 | | D2E2 | |
| Example 12 | C | Example 82 | A | Example 120 | A |
| D2E1 | | D1E1 | | D1E1 | |
| Example 13 | A | Example 82 | C | Example 120 | C |
| D1E1 | | D2E1 | | D2E2 | |
| Example 13 | C | Example 83 | B | Example 121 | A |
| D2E1 | | D1E1 | | D1E1 | |
| Example 14 | A | Example 83 | C | Example 121 | C |
| D1E2 | | D2E1 | | D2E1 | |
| Example 15 | A | Example 84 | B | Example 122 | A |
| D1E1 | | D1E1 | | D1E1 | |
| Example 16 | A | Example 85 | B | Example 122 | C |
| D1E1 | | D1E1 | | D2E1 | |
| Example 17 | A | Example 85 | C | Example 123 | C |
| D1E2 | | D1E2 | | D1E1 | |
| Example 18 | C | Example 85 | C | Example 123 | C |
| D1E2 | | D2E1 | | D2E1 | |
| Example 19 | A | Example 86 | C | Example 124 | B |
| D1E2 | | D1E1 | | D1E2 | |
| Example 20 | B | Example 86 | C | Example 125 | A |
| D1E1 | | D1E2 | | D1E2 | |
| Example 21 | B | Example 88 | C | Example 125 | C |
| D1E1 | | D1E1 | | D2E1 | |
| Example 22 | A | Example 88 | C | Example 126 | C |
| D1E1 | | D1E2 | | D1E1 | |
| Example 23 | B | Example 88 | C | Example 126 | B |
| D1E2 | | D2E1 | | D1E2 | |
| Example 24 | A | Example 89 | A | Example 126 | C |
| D1E1 | | D1E2 | | D2E1 | |
| Example 25 | A | Example 90 | A | Example 127 | A |
| D1E1 | | D1E1 | | D1E1 | |
| Example 26 | B | Example 90 | C | Example 127 | C |
| D1E2 | | D2E2 | | D1E2 | |
| Example 27 | A | Example 91 | A | Example 128 | A |
| D1E1 | | D1E1 | | D1E1 | |
| Example 28 | B | Example 91 | C | Example 128 | C |
| D1E1 | | D1E2 | | D1E2 | |
| Example 29 | A | Example 92 | C | Example 129 | A |
| D1E1 | | D1E1 | | D1E2 | |
| Example 30 | A | Example 93 | B | Example 130 | A |
| D1E1 | | D1E1 | | D1E1 | |
| Example 31 | C | Example 93 | C | Example 132 | B |
| D1E1 | | D1E2 | | D1E2 | |
| Example 32 | C | Example 94 | C | Example 133 | B |
| D1E1 | | D1E1 | | D1E2 | |
| Example 32 | C | Example 95 | A | Example 134 | C |
| D1E2 | | D1E1 | | D1E1 | |
| Example 33 | C | Example 95 | C | Example 135 | C |
| D1E1 | | D1E2 | | D1E1 | |
| Example 34 | C | Example 96 | C | Example 136 | B |
| D1E1 | | D1E1 | | D1E2 | |
| Example 35 | C | Example 96 | A | Example 137 | A |
| D1E1 | | D2E2 | | D1E1 | |
| Example 35 | C | Example 97 | C | Example 138 | B |
| D2E1 | | D1E1 | | D1E1 | |
| Example 36 | C | Example 97 | C | Example 139 | C |
| D1E1 | | D1E2 | | D1E1 | |
| Example 37 | B | Example 98 | C | Example 140 | B |
| D1E1 | | D1E1 | | D1E1 | |
| Example 38 | A | Example 98 | C | Example 141 | C |
| D1E1 | | D1E2 | | D1E1 | |
| Example 38 | C | Example 99 | C | Example 142 | C |
| D2E2 | | D1E1 | | D1E2 | |
| Example 40 | C | Example 99 | C | Example 143 | B |
| D1E2 | | D1E2 | | D1E1 | |
| Example 41 | A | Example 100 | C | Example 144 | C |
| D1E2 | | D1E1 | | D1E2 | |
| Example 42 | B | Example 100 | C | Example 145 | B |
| D1E2 | | D1E2 | | D1E1 | |
| Example 148 | C | Example 101 | B | Example 146 | C |
| D1E1 | | D1E2 | | D1E1 | |
| Example 44 | B | Example 102 | B | Example 147 | B |
| D1E2 | | D1E2 | | D1E1 | |
| Example 45 | C | Example 103 | C | Example 149 | B |
| D1E1 | | D1E1 | | D1E2 | |
| Example 50 | C | Example 103 | B | Example 150 | B |
| D1E1 | | D1E2 | | D1E2 | |
| Example 51 | B | Example 103 | C | Example 151 | B |
| D1E2 | | D2E1 | | D1E1 | |
| Example 52 | B | Example 104 | C | Example 151 | C |
| D1E1 | | D1E1 | | D2E1 | |
| Example 52 | C | Example 104 | C | Example 152 | C |
| D2E1 | | D2E2 | | D1E1 | |
| Example 53 | A | Example 105 | A | Example 152 | C |
| D1E2 | | D1E2 | | D2E1 | |
| Example 57 | B | Example 105 | C | Example 154 | C |
| D1E1 | | D2E1 | | D1E1 | |
| Example 57 | A | Example 106 | B | Example 155 | C |
| D1E2 | | D1E1 | | D1E2 | |
| Example 59 | A | Example 107 | B | Example 155 | B |
| D1E2 | | D1E1 | | D2E1 | |
| Example 60 | B | Example 108 | C | Example 156 | C |
| D1E1 | | D1E1 | | D1E1 | |
| | | | | Example 156 | C |
| | | | | D2E2 | |

### TREX1 Kinetics Assays

Compound binding kinetics were assessed using a pair of TR-FRET assays which measure the proportion of protein bound to a biotinylated TREX1 inhibitor (“probe”).

### Association

N-terminally His-Tev tagged full length human TREX1 (expressed in E. coli and purified in house), complexed with Eu-W1024-anti-6×His (“Eu”; PerkinElmer) in reaction buffer (50 mM Tris, 150 mM NaCl, 2 mM DTT, 0.1 mg/mL BSA, 0.01% (w/v) Tween-20, 5 mM MgCl2, pH 7.4) was combined with an equal volume of test compound in reaction buffer and incubated at 25° C. Concentrations at this stage were 1 nM TREX1/Eu complex, and four concentrations of compound (diluted from 10 mM stocks in 100% DMSO). At defined time points, 18 μL of this mixture was withdrawn and combined with 2 μL probe to a final concentration of 1 μM probe. After incubating for 30 seconds, 18 μL was withdrawn and combined with 2 μL streptavidin-allophycocyanin (“SA-APC”; PerkinElmer) to a final concentration of 1.5 μM SA-APC. Fifteen μL of this mixture was then immediately transferred to a 384-well Black ProxiPlate Plus (PerkinElmer) and read in an EnVision plate reader (PerkinElmer) measuring fluorescence at 615 nm and 665 nm following excitation w/337 nm laser light. Final concentrations were 0.8 nM TREX1/Eu complex, 0.9 μM probe, and 1.5 μM SA-APC.

### Dissociation

N-terminally His-Tev tagged full length human TREX1 (expressed in E. coli and purified in house), complexed with Eu-W1024-anti-6×His (“Eu”; PerkinElmer) in reaction buffer (50 mM Tris, 150 mM NaCl, 2 mM DTT, 0.1 mg/mL BSA, 0.01% (w/v) Tween-20, 5 mM MgCl2, pH 7.4) was combined with an equal volume of test compound in reaction buffer. Concentrations at this stage were 100 nM TREX1/Eu complex and 100 nM test compound (diluted from 10 mM stocks in 100% DMSO). Following an equilibration period of at least an hour at 25° C., this mixture was diluted 100-fold into reaction buffer containing 1 μM probe, and incubated at 25° C. At defined time points, 36 μL of the reaction mixture was withdrawn and combined with 4 μL streptavidin-allophycocyanin (“SA-APC”; PerkinElmer) to a final concentration of 1.5 μM SA-APC. Fifteen μL of this mixture was then immediately transferred to duplicate wells of a 384-well Black ProxiPlate Plus (PerkinElmer) and read in an EnVision plate reader (PerkinElmer) measuring fluorescence at 615 nm and 665 nm following excitation w/337 nm laser light.

### Data Analysis

The TR-FRET signal, a ratio of 665 nm/615 nm emitted light, was converted to fraction enzyme bound to test compound by normalizing to low signal (no enzyme or test compound) and high signal (no test compound) controls. Data from both association and dissociation experiments were fitted globally using Kintek Explorer software, which computes the rate constants directly.

### Kintek Explorer Citations:

Johnson, K. A., Simpson, Z. B., and Blom, T. (2009) Global Kinetic Explorer: A new computer program for dynamic simulation and fitting of kinetic data. Analytical Biochemistry 387, 20-29. http://dx.doi.org/10.1016/j.ab.2008.12.024
Johnson, K. A., Simpson, Z. B., and Blom, T. (2009) FitSpace Explorer: An algorithm to evaluate multi-dimensional parameter space in fitting kinetic data. Analytical Biochemistry 387, 30-41. http://dx.doi.org/10.1016/j.ab.2008.12.025

TABLE 9
Kinetic data for hTREX1.

| | kₒₙ | | | residence |
|---|---|---|---|---|
| | (nM⁻¹ | k_{off} | KD | time |
| | min⁻¹) | (min⁻¹) | (nM) | (mins) |
| Example 2 D1E1 | 0.0118 | 0.00272 | 0.231 | 368 |
| Example 10 D1E1 | 0.0454 | 0.00163 | 0.036 | 613 |
| Example 13 D1E1 | 0.0129 | 0.0027 | 0.209 | 370 |
| Example 14 D1E2 | 0.0132 | 0.00323 | 0.245 | 310 |
| Example 16 D1E1 | 0.00884 | 0.00217 | 0.245 | 461 |
| Example 17 D1E2 | 0.0101 | 0.00283 | 0.28 | 353 |
| Example 22 D1E1 | 0.00635 | 0.0039 | 0.614 | 256 |
| Example 23 D1E2 | 0.0065 | 0.00344 | 0.529 | 290 |
| Example 26 D1E2 | 0.00918 | 0.00372 | 0.405 | 269 |
| Example 38 D1E1 | 0.00981 | 0.00344 | 0.351 | 290 |
| Example 53 D1E2 | 0.00811 | 0.00207 | 0.255 | 483 |
| Example 77 D1E1 | 0.0138 | 0.00466 | 0.338 | 215 |
| Example 78 D1E1 | — | 0.00242 | — | 413 |
| Example 81 D1E2 | — | 0.00140 | — | 714 |
| Example 91 D1E1 | — | 0.00055 | — | 1825 |
| Example 105 D1E2 | — | 0.00151 | — | 662 |
| Example 111 D1E2 | — | 0.00217 | — | 461 |
| Example 115 D1E2 | — | 0.00194 | — | 515 |
| Example 122 D1E1 | — | 0.00069 | — | 1451 |
| Example 124 D1E2 | — | 0.00135 | — | 741 |
| Example 125 D1E2 | — | 0.00184 | — | 543 |
| Example 149 D1E2 | — | 0.00198 | — | 505 |
| Example 150 D1E2 | — | 0.00548 | — | 182 |
| Example 46 D1E2 | — | 0.00289 | — | 346 |
| Example 57 D1E2 | — | 0.00232 | — | 431 |
| Example 59 D1E2 | — | 0.00194 | — | 515 |
| Example 67 D1E2 | — | 0.00245 | — | 408 |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 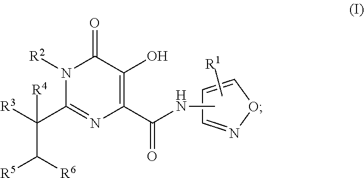
or a pharmaceutically acceptable salt thereof, wherein:
R¹ is halo, hydrogen, (C₁-C₄)alkyl, or halo(C₁-C₄)alkyl;
R² is hydrogen, (C₁-C₄)alkyl, halo(C₁-C₄)alkyl, —(C₁-C₄)alkylOR^{a}, —(C₁-C₄)alkylSR^{a}, or —(C₁-C₄)alkylNR^{b}R^{c};
R^{a} is selected from hydrogen, (C₁-C₄)alkyl, halo(C₁-C₄)alkyl, —COOR^{b}, and —C(O)NR^{b}R^{c};
R^{b} and R^{c} are each independently hydrogen or (C₁-C₄)alkyl;
R³ and R⁴ are each independently hydrogen, halo, (C₁-C₄)alkyl, or halo(C₁-C₄)alkyl;
R⁵ is phenyl, 5 to 7-membered heteroaryl, or 5 to 7-membered heterocyclyl, each of which being optionally substituted with 1 to 3 groups selected from R⁷;
R⁶ is 5 to 7-membered heteroaryl or 5 to 7-membered heterocyclyl, each of which being optionally substituted with 1 to 3 groups selected from R⁸; and
R⁷ and R⁸ are each independently selected from halo, hydroxyl, (C₁-C₄)alkyl, (C₁-C₄)deuteroalkyl, halo(C₁-C₄)alkyl, (C₁-C₄)alkoxy, halo(C₁-C₄)alkoxy, —(C₁-C₄)alkylOR^{a}, —(C₁-C₄)alkylSR^{a}, —(C₁-C₄)alkylNR^{b}R^{c}, —(C₁-C₄)alkyl-cyano, —(C₁-C₄)alkylC(O)NR^{b}R^{c}, cyano, —[(C₁-C₄)alkyl(4- to 7-membered heterocyclyl)], -(4- to 7-membered heterocyclyl), —[(C₁-C₄)alkyl(C₃-C₅)cycloalkyl], —C(O)NR^{b}R^{c}, —COR^{b}, and —COOR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}.

2. The compound of claim 1, wherein the compound is of the Formula II: 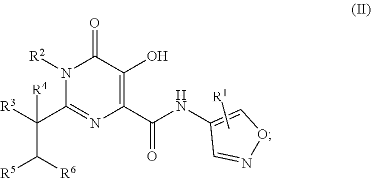
or a pharmaceutically acceptable salt thereof.

3. The compound of claim 1 or 2, or a pharmaceutically acceptable salt thereof, wherein R¹ is hydrogen.

4. The compound of any one of claims 1 to 3, or a pharmaceutically acceptable salt thereof, wherein R² is (C₁-C₄)alkyl.

5. The compound of any one of claims 1 to 4, or a pharmaceutically acceptable salt thereof, wherein R³ is halo, hydrogen or (C₁-C₄)alkyl.

6. The compound of any one of claims 1 to 5, or a pharmaceutically acceptable salt thereof, wherein R³ is halo or hydrogen.

7. The compound of any one of claims 1 to 6, or a pharmaceutically acceptable salt thereof, wherein R³ is hydrogen.

8. The compound of any one of claims 1 to 7, or a pharmaceutically acceptable salt thereof, wherein R⁴ is hydrogen, (C₁-C₄)alkyl, or halo(C₁-C₄)alkyl.

9. The compound of any one of claims 1 to 8, or a pharmaceutically acceptable salt thereof, wherein R⁴ is (C₁-C₄)alkyl or halo(C₁-C₄)alkyl.

10. The compound of any one of claims 1 to 9, or a pharmaceutically acceptable salt thereof, wherein R⁴ is (C₁-C₄)alkyl.

11. The compound of any one of claims 1 to 10, or a pharmaceutically acceptable salt thereof, wherein R⁵ is phenyl or 5 to 7-membered heteroaryl, each of which being optionally substituted with 1 to 3 groups selected from R⁷.

12. The compound of any one of claims 1 to 11, or a pharmaceutically acceptable salt thereof, wherein R⁵ is phenyl or pyridinyl, each of which being optionally substituted with 1 to 3 groups selected from R⁷.

13. The compound of any one of claims 1 to 12, or a pharmaceutically acceptable salt thereof, wherein R⁵ is phenyl optionally substituted with 1 to 3 groups selected from R⁷.

14. The compound of any one of claims 1 to 13, or a pharmaceutically acceptable salt thereof, wherein R⁶ is 5 to 7-membered heteroaryl optionally substituted with 1 to 3 groups selected from R⁸.

15. The compound of any one of claims 1 to 14, or a pharmaceutically acceptable salt thereof, wherein R⁶ is pyridinyl, oxadiazolyl, triazolyl, tetrazolyl, isoxazolyl, imidazolyl, pyrazolyl, pyrimidinyl, or pyrazinyl, each of which being optionally substituted with 1 to 3 groups selected from R⁸.

16. The compound of any one of claims 1 to 15, or a pharmaceutically acceptable salt thereof, wherein R⁶ is pyrazolyl, pyrimidinyl, or pyrazinyl, optionally substituted with 1 to 3 groups selected from R⁸.

17. The compound of any one of claims 1 to 16, or a pharmaceutically acceptable salt thereof, wherein R⁶ is pyrazolyl optionally substituted with 1 to 3 groups selected from R⁸.

18. The compound of any one of claims 1 to 16, or a pharmaceutically acceptable salt thereof, wherein R⁶ is pyrimidinyl optionally substituted with 1 to 3 groups selected from R⁸.

19. The compound of any one of claims 1 to 16, or a pharmaceutically acceptable salt thereof, wherein R⁶ is pyrazinyl optionally substituted with 1 to 3 groups selected from R⁸.

20. The compound of any one of claims 1 to 19, or a pharmaceutically acceptable salt thereof, wherein R⁷ and R⁸ are each independently selected from halogen, hydroxyl, (C₁-C₄)alkyl, halo(C₁-C₄)alkyl, —(C₁-C₄)alkylOR^{a}, cyano, —(C₁-C₄)alkylNR^{b}R^{c}, —[(C₁-C₄)alkyl(4- to 7-membered heterocyclyl)], —[(C₁-C₄)alkyl(C₃-C₅)cycloalkyl], —(C₁-C₄)alkylNR^{b}R^{c}, —(C₁-C₄)alkyl-cyano, -(4- to 7-membered heterocyclyl), —C(O)NR^{b}R^{c}, and —COR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}.

21. The compound of any one of claims 1 to 20, or a pharmaceutically acceptable salt thereof, wherein R⁷ is selected from halo, (C₁-C₄)alkyl, hydroxyl, halo(C₁-C₄)alkyl, cyano, and —C(O)NR^{b}R^{c}.

22. The compound of any one of claims 1 to 21, or a pharmaceutically acceptable salt thereof, wherein R⁷ is selected from halo and cyano.

23. The compound of any one of claims 1 to 22, or a pharmaceutically acceptable salt thereof, wherein R⁸ is selected from halo, (C₁-C₄)alkyl, halo(C₁-C₄)alkyl, —(C₁-C₄)alkylOR^{a}, —(C₁-C₄)alkylNR^{b}R^{c}, —[(C₁-C₄)alkyl(4- to 7-membered heterocyclyl)], —[(C₁-C₄)alkyl(C₃-C₅)cycloalkyl], —(C₁-C₄)alkyl-cyano, -(4- to 7-membered heterocyclyl), —(C₁-C₄)alkylNR^{b}R^{c}, and —COR^{b}, wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}.

24. The compound of any one of claims 1 to 23, or a pharmaceutically acceptable salt thereof, wherein R⁸ is selected from halo, (C₁-C₄)alkyl, halo(C₁-C₄)alkyl, —(C₁-C₄)alkylOR^{a}, —(C₁-C₄)alkylNR^{b}R^{c}, —[(C₁-C₄)alkyl(morpholinyl)], —[(C₁-C₄)alkyl(piperizinyl)], —[(C₁-C₄)alkylcyclopropyl], —(C₁-C₄)alkyl-cyano, -(4- to 7-membered heterocyclyl), —(C₁-C₄)alkylNR^{b}R^{c}, and —COR^{b}, wherein said morpholinyl, piperizinyl, and cyclopropyl are each optionally substituted with 1 to 3 groups selected from halo, (C₁-C₄)alkyl, halo(C₁-C₄)alkyl, (C₁-C₄)alkoxy, halo(C₁-C₄)alkoxy, COOR^{b}, —C(O)NR^{b}R^{c}, and —COR^{b}.

25. The compound of claim 1, wherein the compound is of the Formula III: 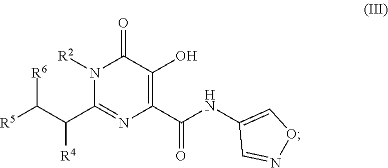
or a pharmaceutically acceptable salt thereof, wherein
R² is (C₁-C₄)alkyl;
R⁴ is (C₁-C₄)alkyl;
R⁵ is phenyl substituted with 1 or 2 groups selected from R⁷,
R⁶ is pyrazolyl, pyrimidinyl or pyrazinyl, optionally substituted with 1 to 3 groups selected from R⁸;
R⁷ is halo, halo(C₁-C₄)alkyl, or cyano;
R⁸ is halo, (C₁-C₄)alkyl, halo(C₁-C₄)alkyl, —(C₁-C₄)alkylOR^{a}, —(C₁-C₄)alkyl-cyano, cyano, —[(C₁-C₄)alkyl(4- to 7-membered heterocyclyl)], -(4- to 7-membered heterocyclyl), —[(C₁-C₄))alkyl(C₃-C₅)cycloalkyl], wherein said 4- to 7-membered heterocyclyl and (C₃-C₅)cycloalkyl are each optionally substituted with 1 to 3 groups selected from halo, (C₁-C₄)alkyl, halo(C₁-C₄)alkyl, (C₁-C₄)alkoxy, and halo (C₁-C₄)alkoxy; and
R^{a} is (C₁-C₄)alkyl or halo (C₁-C₄)alkyl.

26. The compound of any one of claims 1 to 25, or a pharmaceutically acceptable salt thereof, wherein at least one R⁷, if present, is present at the ortho position.

27. The compound of any one of claims 1 to 25, or a pharmaceutically acceptable salt thereof, wherein at least one R⁷, if present, is chloro or cyano.

28. The compound of claim 1, wherein the compound is selected from
2-((1S,2R)-1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(6-methylpyridin-3-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(6-methylpyridin-3-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(6-methylpyridin-3-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(6-methylpyridin-3-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(2-methylpyridin-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(2-methylpyridin-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(2-methylpyridin-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(2-methylpyridin-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanopyridin-3-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanopyridin-3-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanopyridin-3-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanopyridin-3-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(3-cyanopyridin-4-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(3-cyanopyridin-4-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(3-cyanopyridin-4-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(3-cyanopyridin-4-yl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(3-cyano-1-methyl-1H-pyrazol-4-yl)-1-(2-cyanophenyl)propan-2-yl)-5-hydroxy-N-(isoxazol-4-yl)-1-methyl-6-oxo-1,6-dihydropyrimidine-4-carboxamide,
2-((1S,2S)-1-(3-cyano-1-methyl-1H-pyrazol-4-yl)-1-(2-cyanophenyl)propan-2-yl)-5-hydroxy-N-(isoxazol-4-yl)-1-methyl-6-oxo-1,6-dihydropyrimidine-4-carboxamide,
2-((1S,2R)-1-(3-cyano-1-methyl-1H-pyrazol-4-yl)-1-(2-cyanophenyl)propan-2-yl)-5-hydroxy-N-(isoxazol-4-yl)-1-methyl-6-oxo-1,6-dihydropyrimidine-4-carboxamide,
2-((1R,2S)-1-(3-cyano-1-methyl-1H-pyrazol-4-yl)-1-(2-cyanophenyl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2,6-dicyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2,6-dicyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2,6-dicyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2,6-dicyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-morpholinoethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-morpholinoethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-morpholinoethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-morpholinoethyl)-114,212-pyrazol-4-yl)propan-2-yl)-5-hydroxy-N-(isoxazol-4-yl)-1-methyl-6-oxo-1,6-dihydropyrimidine-4-carboxamide,
tert-butyl 4-(2-(4-((1S,2R)-1-(2-cyanophenyl)-2-(5-hydroxy-4-(isoxazol-4-ylcarbamoyl)-1-methyl-6-oxo-1,6-dihydropyrimidin-2-yl)propyl)-1H-pyrazol-1-yl)ethyl)piperazine-1-carboxylate,
tert-butyl 4-(2-(4-((1R,2S)-1-(2-cyanophenyl)-2-(5-hydroxy-4-(isoxazol-4-ylcarbamoyl)-1-methyl-6-oxo-1,6-dihydropyrimidin-2-yl)propyl)-1H-pyrazol-1-yl)ethyl)piperazine-1-carboxylate,
tert-butyl 4-(2-(4-((1S,2S)-1-(2-cyanophenyl)-2-(5-hydroxy-4-(isoxazol-4-ylcarbamoyl)-1-methyl-6-oxo-1,6-dihydropyrimidin-2-yl)propyl)-1H-pyrazol-1-yl)ethyl)piperazine-1-carboxylate,
tert-butyl 4-(2-(4-((1R,2R)-1-(2-cyanophenyl)-2-(5-hydroxy-4-(isoxazol-4-ylcarbamoyl)-1-methyl-6-oxo-1,6-dihydropyrimidin-2-yl)propyl)-1H-pyrazol-1-yl)ethyl)piperazine-1-carboxylate,
2-((1S,2R)-1-(2-cyanophenyl)-1-(1-(2,2,2-trifluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2,2,2-trifluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2,2,2-trifluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2,2,2-trifluoroethyl)-1H-pyrazol-4-yl)propan-2-yl)-5-hydroxy-N-(isoxazol-4-yl)-1-methyl-6-oxo-1,6-dihydropyrimidine-4-carboxamide
2-((1R,2R)-1-(2-cyanophenyl)-1-(1-(cyclopropylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(cyclopropylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(cyclopropylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(cyclopropylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(3-methoxypropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(3-methoxypropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(3-methoxypropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(3-methoxypropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(5-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(5-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(5-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(5-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(4-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(4-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(4-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(4-chloro-2-cyanophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-4,5-difluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-(trifluoromethyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-(piperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-(4-methylpiperazin-1-yl)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-(dimethylamino)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-(dimethylamino)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-(dimethylamino)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-(dimethylamino)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(3-(dimethylamino)propyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(3-(dimethylamino)propyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(3-(dimethylamino)propyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(3-(dimethylamino)propyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-hydroxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-hydroxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-hydroxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-hydroxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(difluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(difluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(difluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(difluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-1H-imidazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-1H-imidazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-1H-imidazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-1H-imidazol-4-yl)propan-2-yl)-5-hydroxy-N-(isoxazol-4-yl)-1-methyl-6-oxo-1,6-dihydropyrimidine-4-carboxamide,
2-((2R,3S)-3-(2-cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((2S,3R)-3-(2-cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((2S,3S)-3-(2-cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((2R,3R)-3-(2-cyanophenyl)-1,1,1-trifluoro-3-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((2R,3S)-3-(2-cyanophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2S,3R)-3-(2-cyanophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2S,3S)-3-(2-cyanophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2R,3R)-3-(2-cyanophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2S,3S)-3-(2-cyano-5-fluorophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2R,3R)-3-(2-cyano-5-fluorophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2S,3R)-3-(2-cyano-5-fluorophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
2-((2R,3S)-3-(2-cyano-5-fluorophenyl)-3-(1,3-dimethyl-1H-pyrazol-4-yl)-1,1,1-trifluoropropan-2-yl)-5-hydroxy-N-(isoxazol-4-yl)-1-methyl-6-oxo-1,6-dihydropyrimidine-4-carboxamide,
(R)-2-(2-(2-cyanophenyl)-1,1-difluoro-2-(1-methyl-1H-pyrazol-4-yl)ethyl)-5-hydroxy-N-(isoxazol-4-yl)-1-methyl-6-oxo-1,6-dihydropyrimidine-4-carboxamide,
(S)-2-(2-(2-cyanophenyl)-1,1-difluoro-2-(1-methyl-1H-pyrazol-4-yl)ethyl)-5-hydroxy-N-(isoxazol-4-yl)-1-methyl-6-oxo-1,6-dihydropyrimidine-4-carboxamide,
2-((1R,2R)-1-(2-chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide
2-((1R,2S)-1-(2-chlorophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(3-cyano-1-methyl-1H-pyrazol-4-yl)-1-(2,5-difluorophenyl)propan-2-yl)-5-hydroxy-N-(isoxazol-4-yl)-1-methyl-6-oxo-1,6-dihydropyrimidine-4-carboxamide,
2-((1R,2S)-1-(3-cyano-1-methyl-1H-pyrazol-4-yl)-1-(2,5-difluorophenyl)propan-2-yl)-5-hydroxy-N-(isoxazol-4-yl)-1-methyl-6-oxo-1,6-dihydropyrimidine-4-carboxamide,
2-((1R,2R)-1-(3-cyano-1-methyl-1H-pyrazol-4-yl)-1-(2,5-difluorophenyl)propan-2-yl)-5-hydroxy-N-(isoxazol-4-yl)-1-methyl-6-oxo-1,6-dihydropyrimidine-4-carboxamide,
2-((1S,2S)-1-(3-cyano-1-methyl-1H-pyrazol-4-yl)-1-(2,5-difluorophenyl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(oxazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(oxazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(oxazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(oxazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-1H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-1H-1,2,4-triazol-3-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-1H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-1H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2R)-1-(4-cyano-2-methyl-1l2,2l4-pyrazol-3-yl)-1-(2-cyanophenyl)propan-2-yl)-5-hydroxy-N-(isoxazol-4-yl)-1-methyl-6-oxo-1,6-dihydropyrimidine-4-carboxamide,
2-((1S,2S)-1-(4-cyano-2-methyl-1l2,2l4-pyrazol-3-yl)-1-(2-cyanophenyl)propan-2-yl)-5-hydroxy-N-(isoxazol-4-yl)-1-methyl-6-oxo-1,6-dihydropyrimidine-4-carboxamide,
2-((1R,2S)-1-(4-cyano-2-methyl-1l2,2l4-pyrazol-3-yl)-1-(2-cyanophenyl)propan-2-yl)-5-hydroxy-N-(isoxazol-4-yl)-1-methyl-6-oxo-1,6-dihydropyrimidine-4-carboxamide,
2-((1S,2R)-1-(4-cyano-2-methyl-1l2,2l4-pyrazol-3-yl)-1-(2-cyanophenyl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(6-methylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(6-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(6-methylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(6-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-fluorophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-fluorophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-fluorophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(3-cyano-1-methyl-1H-pyrazol-4-yl)-1-(2-cyano-5-fluorophenyl)propan-2-yl)-5-hydroxy-N-(isoxazol-4-yl)-1-methyl-6-oxo-1,6-dihydropyrimidine-4-carboxamide,
2-((1S,2S)-1-(3-cyano-1-methyl-1H-pyrazol-4-yl)-1-(2-cyano-5-fluorophenyl)propan-2-yl)-5-hydroxy-N-(isoxazol-4-yl)-1-methyl-6-oxo-1,6-dihydropyrimidine-4-carboxamide,
2-((1S,2R)-1-(3-cyano-1-methyl-1H-pyrazol-4-yl)-1-(2-cyano-5-fluorophenyl)propan-2-yl)-5-hydroxy-N-(isoxazol-4-yl)-1-methyl-6-oxo-1,6-dihydropyrimidine-4-carboxamide,
2-((1R,2S)-1-(3-cyano-1-methyl-1H-pyrazol-4-yl)-1-(2-cyano-5-fluorophenyl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1,3-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,4-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,4-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,4-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,4-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-fluorophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-fluorophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-fluorophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-fluorophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(pyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(pyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(pyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(pyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(2-methylpyrimidin-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-ethyl-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-ethyl-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-4,5-difluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-4,5-difluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-4,5-difluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-4,5-difluorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-4,5-difluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-4,5-difluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-4,5-difluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-4,5-difluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-4-fluorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-(dimethylcarbamoyl)phenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(methyl-d3)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-(1-(2-cyanophenyl)-1-(1-(methyl-d3)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-(1-(2-cyanophenyl)-1-(1-(methyl-d3)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-(1-(2-cyanophenyl)-1-(1-(methyl-d3)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(2-methyl-2H-tetrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(2-methyl-2H-tetrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(2-methyl-2H-tetrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(2-methyl-2H-tetrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-4-hydroxyphenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-4-hydroxyphenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-4-hydroxyphenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-4-hydroxyphenyl)-1-(1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(2-methylpyrimidin-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(2-methylpyrimidin-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(2-methylpyrimidin-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(2-methylpyrimidin-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1,4-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1,4-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1,4-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1,4-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(1-ethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide
2-((1R,2R)-1-(2-chlorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(3-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-3-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-3-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-3-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-3-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-hydroxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-methoxy-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(oxetan-3-ylmethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(5-cyano-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-methoxyethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-cyano-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-cyano-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-cyano-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-cyano-2-methylpropyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(1-cyano-2-methylpropan-2-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(1-cyano-2-methylpropan-2-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(1-cyano-2-methylpropan-2-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(1-cyano-2-methylpropan-2-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2-(trifluoromethoxy)ethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2,2-difluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2,2-difluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2,2-difluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2,2-difluoroethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-(oxetan-3-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(oxetan-3-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(oxetan-3-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(oxetan-3-yl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-methoxyethyl)-3-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1-(2-methoxyethyl)-5-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1-(2-methoxyethyl)-3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1-(2-methoxyethyl)-3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1-(2-methoxyethyl)-3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1-(2-methoxyethyl)-3,5-dimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1,3-dimethyl-1H-pyrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1,5-dimethyl-1H-pyrazol-3-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(5-methylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(5-(trifluoromethyl)pyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(5-(trifluoromethyl)pyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(5-(trifluoromethyl)pyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(5-(trifluoromethyl)pyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(3,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3,5,6-trimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3,5,6-trimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3,5,6-trimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3,5,6-trimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3,5-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3,5-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3,5-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3,5-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(5,6-dimethylpyrazin-2-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3-fluoro-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3-fluoro-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3-fluoro-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3-fluoro-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3-fluoro-1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3-fluoro-1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3-fluoro-1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3-fluoro-1-isopropyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-cyano-5-fluorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyano-5-fluorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyano-5-fluorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyano-5-fluorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3-fluoro-1-methyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-1H-tetrazol-5-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-1H-tetrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-1H-tetrazol-5-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-1H-tetrazol-5-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-ethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-ethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-ethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-ethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-isopropyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-isopropyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-isopropyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-isopropyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(3,5-dimethyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-(trifluoromethyl)-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-(trifluoromethyl)-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-(trifluoromethyl)-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-(trifluoromethyl)-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5-cyano-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(5-cyano-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5-cyano-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5-cyano-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-methyl-1H-imidazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-methyl-1H-imidazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-methyl-1H-imidazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-methyl-1H-imidazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(4-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(5-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(3-methyl-1H-pyrazol-1-yl)propan-2-yl)-5-hydroxy-N-(isoxazol-4-yl)-1-methyl-6-oxo-1,6-dihydropyrimidine-4-carboxamide,
2-((1R,2R)-1-(5-carbamoyl-1H-pyrazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1S,2S)-1-(5-carbamoyl-1H-pyrazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1R,2S)-1-(5-carbamoyl-1H-pyrazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1S,2R)-1-(5-carbamoyl-1H-pyrazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-1H-pyrazol-4-yl)propan-2-yl)-1-ethyl-5-hydroxy-N-(isoxazol-4-yl)-6-oxo-1,6-dihydropyrimidine-4-carboxamide,
2-((1R,2S)-1-(2-cyanophenyl)-1-(1-methyl-1H-pyrazol-4-yl)propan-2-yl)-1-ethyl-5-hydroxy-N-(isoxazol-4-yl)-6-oxo-1,6-dihydropyrimidine-4-carboxamide,
2-((1S,2S)-1-(2-cyanophenyl)-1-(1-methyl-1H-pyrazol-4-yl)propan-2-yl)-1-ethyl-5-hydroxy-N-(isoxazol-4-yl)-6-oxo-1,6-dihydropyrimidine-4-carboxamide,
2-((1R,2S)-1-(2-cyanophenyl)-1-(1-methyl-1H-pyrazol-4-yl)propan-2-yl)-1-ethyl-5-hydroxy-N-(isoxazol-4-yl)-6-oxo-1,6-dihydropyrimidine-4-carboxamide,
2-((1S,2R)-1-(2-cyanophenyl)-1-(1-methyl-1H-pyrazol-4-yl)propan-2-yl)-5-hydroxy-1-isopropyl-N-(isoxazol-4-yl)-6-oxo-1,6-dihydropyrimidine-4-carboxamide,
2-((1R,2S)-1-(2-cyanophenyl)-1-(1-methyl-1H-pyrazol-4-yl)propan-2-yl)-5-hydroxy-1-isopropyl-N-(isoxazol-4-yl)-6-oxo-1,6-dihydropyrimidine-4-carboxamide,
2-((1S,2S)-1-(2-cyanophenyl)-1-(1-methyl-1H-pyrazol-4-yl)propan-2-yl)-5-hydroxy-1-isopropyl-N-(isoxazol-4-yl)-6-oxo-1,6-dihydropyrimidine-4-carboxamide,
2-((1R,2R)-1-(2-cyanophenyl)-1-(1-methyl-1H-pyrazol-4-yl)propan-2-yl)-5-hydroxy-1-isopropyl-N-(isoxazol-4-yl)-6-oxo-1,6-dihydropyrimidine-4-carboxamide,
2-((1R,2R)-1-(2-cyanophenyl)-1-(1-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-(trifluoromethyl)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(4-methyl-4H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(4-methyl-4H-1,2,4-triazol-3-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(4-methyl-4H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(4-methyl-4H-1,2,4-triazol-3-yl)propan-2-yl)-5-hydroxy-N-(isoxazol-4-yl)-1-methyl-6-oxo-1,6-dihydropyrimidine-4-carboxamide,
2-((1R,2S)-1-(2-chloro-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-chloro-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1R,2R)-1-(2-chloro-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-chloro-5-fluorophenyl)-1-(1,3,5-trimethyl-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanopyridin-3-yl)-1-phenylpropan-2-yl)-5-hydroxy-N-(isoxazol-4-yl)-1-methyl-6-oxo-1,6-dihydropyrimidine-4-carboxamide,
2-((1R,2S)-1-(2-cyanopyridin-3-yl)-1-phenylpropan-2-yl)-5-hydroxy-N-(isoxazol-4-yl)-1-methyl-6-oxo-1,6-dihydropyrimidine-4-carboxamide,
2-((1R,2R)-1-(2-cyanopyridin-3-yl)-1-phenylpropan-2-yl)-5-hydroxy-N-(isoxazol-4-yl)-1-methyl-6-oxo-1,6-dihydropyrimidine-4-carboxamide,
2-((1S,2S)-1-(2-cyanopyridin-3-yl)-1-phenylpropan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(5-methyl-1,3,4-oxadiazol-2-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(5-methyl-1,3,4-oxadiazol-2-yl)propan-2-yl)-5-hydroxy-N-(isoxazol-4-yl)-1-methyl-6-oxo-1,6-dihydropyrimidine-4-carboxamide,
2-((1R,2R)-1-(2-chlorophenyl)-1-(5-methyl-1,3,4-oxadiazol-2-yl)propan-2-yl)-5-hydroxy-N-(isoxazol-4-yl)-1-methyl-6-oxo-1,6-dihydropyrimidine-4-carboxamide, and
2-((1S,2S)-1-(2-chlorophenyl)-1-(5-methyl-1,3,4-oxadiazol-2-yl)propan-2-yl)-5-hydroxy-N-(isoxazol-4-yl)-1-methyl-6-oxo-1,6-dihydropyrimidine-4-carboxamide;
or a pharmaceutically acceptable salt of any of the foregoing.

29. A compound selected from
2-((1R,2R)-1-(2-chlorophenyl)-1-(2H-indazol-2-yl)propan-2-yl)-5-hydroxy-N-(isoxazol-4-yl)-1-methyl-6-oxo-1,6-dihydropyrimidine-4-carboxamide,
2-((1S,2S)-1-(2-chlorophenyl)-1-(2H-indazol-2-yl)propan-2-yl)-5-hydroxy-N-(isoxazol-4-yl)-1-methyl-6-oxo-1,6-dihydropyrimidine-4-carboxamide,
2-((1R,2S)-1-(2-chlorophenyl)-1-(2H-indazol-2-yl)propan-2-yl)-5-hydroxy-N-(isoxazol-4-yl)-1-methyl-6-oxo-1,6-dihydropyrimidine-4-carboxamide,
2-((1S,2R)-1-(2-chlorophenyl)-1-(2H-indazol-2-yl)propan-2-yl)-5-hydroxy-N-(isoxazol-4-yl)-1-methyl-6-oxo-1,6-dihydropyrimidine-4-carboxamide,
2-((1R,2R)-1-(1H-benzo[d]imidazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1S,2S)-1-(1H-benzo[d]imidazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1R,2S)-1-(1H-benzo[d]imidazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1S,2R)-1-(1H-benzol[d]imidazol-1-yl)-1-(2-chlorophenyl)propan-2-yl)-5-hydroxy-N-(isoxazol-4-yl)-1-methyl-6-oxo-1,6-dihydropyrimidine-4-carboxamide,
2-((1R,2R)-1-(2-cyanophenyl)-1-(1-methyl-3-(N-methylacetamido)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2S)-1-(2-cyanophenyl)-1-(1-methyl-3-(N-methylacetamido)-1H-pyrazol-4-yl)propan-2-yl)-5-hydroxy-N-(isoxazol-4-yl)-1-methyl-6-oxo-1,6-dihydropyrimidine-4-carboxamide,
2-((1R,2S)-1-(2-cyanophenyl)-1-(1-methyl-3-(N-methylacetamido)-1H-pyrazol-4-yl)propan-2-yl)-5-hydroxy-N-(isoxazol-4-yl)-1-methyl-6-oxo-1,6-dihydropyrimidine-4-carboxamide,
2-((1S,2R)-1-(2-cyanophenyl)-1-(1-methyl-3-(N-methylacetamido)-1H-pyrazol-4-yl)propan-2-yl)-5-hydroxy-N-(isoxazol-4-yl)-1-methyl-6-oxo-1,6-dihydropyrimidine-4-carboxamide;
or a pharmaceutically acceptable salt of any of the foregoing.

30. A pharmaceutical composition comprising the compound of any one of claims 1 to 29, or a pharmaceutically acceptable salt thereof; and a pharmaceutically acceptable carrier.

31. A method of treating a disease responsive to the inhibition of TREX1 in a subject, comprising administering to the subject, a therapeutically effective amount of a compound of any one of claims 1 to 29, or a pharmaceutically acceptable salt thereof, or the composition of claim 30.

32. The method of claim 31, wherein the disease is cancer.
